(19) United States
(12) Patent Application Publication
(10) Pub. No.: US 2016/0137639 A1
(43) Pub. Date: May 19, 2016

(54) TRIAZOLE-ISOXAZOLE COMPOUND AND MEDICAL USE THEREOF

(71) Applicant: JAPAN TOBACCO INC., Tokyo (JP)

(72) Inventors: Masayuki KOTOKU, Takatsuki-shi, Osaka (JP); Takaki MAEBA, Takatsuki-shi, Osaka (JP); Noriyoshi SEKI, Takatsuki-shi, Osaka (JP); Shintaro HIRASHIMA, Takatsuki-shi, Osaka (JP); Shingo FUJIOKA, Takatsuki-shi, Osaka (JP); Shingo OBIKA, Takatsuki-shi, Osaka (JP); Hiroshi YAMANAKA, Takatsuki-shi, Osaka (JP); Masahiro YOKOTA, Takatsuki-shi, Osaka (JP); Takayuki SAKAI, Takatsuki-shi, Osaka (JP); Kazuyuki HIRATA, Takatsuki-shi, Osaka (JP); Katsuya MAEDA, Takatsuki-shi, Osaka (JP); Makoto SHIOZAKI, Takatsuki-shi, Osaka (JP); Yuko SHINAGAWA, Takatsuki-shi, Osaka (JP); Taku IKENOGAMI, Takatsuki-shi, Osaka (JP); Satoki DOI, Takatsuki-shi, Osaka (JP); Takahiro OKA, Takatsuki-shi, Osaka (JP); Takuya MATSUO, Takatsuki-shi, Osaka (JP); Yoshihiro SUWA, Takatsuki-shi, Osaka (JP); Keisuke ITO, Takatsuki-shi, Osaka (JP); Satoru NOJI, Takatsuki-shi, Osaka (JP); Yoshinori HARA, Takatsuki-shi, Osaka (JP)

(21) Appl. No.: 14/438,553

(22) PCT Filed: Oct. 25, 2013
(86) PCT No.: PCT/JP2013/079016

§ 371 Date: Feb. 1, 2016

Related U.S. Application Data
(60) Provisional application No. 61/719,097, filed on Oct. 26, 2012.

(30) Foreign Application Priority Data
Oct. 26, 2012 (JP) 2012-236162

(51) Int. Cl. C07D 471/04; C07D 413/04; C07D 403/04; C07D 249/08; C07D 401/12; C07D 403/12; C07D 261/08; C07D 417/04

(52) U.S. Cl. CPC C07D 471/04; C07D 261/08; C07D 413/04; C07D 417/04; C07D 249/08; C07D 401/12; C07D 403/12; C07D 403/04

(57) ABSTRACT

A compound represented by Formula [I]:

or pharmaceutically acceptable salt thereof, wherein each symbol is as defined in the description.

### TECHNICAL FIELD

The present invention relates to triazole compounds and medicinal use thereof. The present invention relates to isoxazole compounds and medicinal use thereof.

In particular, the present invention relates to compounds which can inhibit retinoid-related orphan receptor gamma (RORγ), thereby the differentiation and activation of T helper 17 (Th17) cells can be inhibited, and the production of interleukin-17 (IL-17) can be inhibited.

Specifically, the present invention relates to compounds for preventing or treating a disease related to Th17 cells, for example, autoimmune disease such as rheumatoid arthritis, psoriasis, inflammatory bowel disease such as Crohn's disease and ulcerative colitis, multiple sclerosis, systemic lupus erythematosus (SLE), ankylosing spondylitis, uveitis, polymyalgia rheumatica, and type I diabetes; allergic disease such as asthma; dry eye; fibrosis such as pulmonary fibrosis and primary biliary cirrhosis; and metabolic disease such as diabetes and medicinal use thereof.

### BACKGROUND ART

RORγ is a nuclear receptor which is important for the differentiation and activation of Th17 cells. RORγt is also known as a splice variant of RORγ. RORγ and RORγt differ only in their N-terminal domains, and share the same ligand-binding domain and DNA-binding domain. It is reported that RORγ is expressed in other tissues besides Th17 cells. By inhibiting RORγ, the differentiation and activation of Th17 cells can be inhibited. IL-17 produced in Th17 cells is involved in the induction of a variety of chemokines, cytokines, metalloproteases and other inflammatory mediators, and the migration of neutrophil, hence, the inhibition of IL-17 may lead to the inhibition of such induction and migration. RORγ in adipose tissues is related to the regulation of adipogenesis, and by inhibiting RORγ, insulin resistance can be improved.

It is known that Th17 cells are involved in autoimmune disease such as rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica, and type I diabetes; allergic disease; dry eye; and fibrosis such as pulmonary fibrosis and primary biliary cirrhosis.

As for rheumatoid arthritis, for example, it is reported that the administration of anti-IL-17 antibody can improve swelling and joint destruction associated with collagen-induced arthritis. Moreover, it is reported that swelling and joint destruction associated with collagen-induced arthritis can be improved in IL-17-deficient mice.

As for psoriasis, it is reported that in a clinical trial, the administration of anti-IL-17 antibody is effective in treating psoriasis.

As for inflammatory bowel disease such as Crohn's disease and ulcerative colitis, in a colitis model induced by the adaptive transfer of naive T-cells, the adaptive transfer of naive T-cells derived from RORγ-KO mice does not increase IL-17 in the mucosa, thereby the onset of colitis can be suppressed.

As for multiple sclerosis, the disease state of mouse experimental autoimmune encephalomyelitis model which is an animal model of multiple sclerosis can be suppressed in RORγt-KO mice.

As for systemic lupus erythematosus, it is reported that the onset of GBM nephritis model which is an animal model of glomerulonephritis can be inhibited in RORγt-KO mice. Nephritis associated with SLE may also be suppressed.

As for ankylosing spondylitis, it is reported that the administration of anti-IL-17 antibody is effective in treating ankylosing spondylitis.

As for uveitis, it is reported that the administration of anti-IL-17 antibody is effective in treating uveitis associated with Behcet's disease, sarcoidosis and Harada disease.

As for polymyalgia rheumatica, an efficacy of anti-IL-antibody in treatment of polymyalgia rheumatica is currently tested in a clinical trial.

As for type I diabetes, the disease state of NOD mice which is a type I diabetes model can be suppressed by the administration of anti-IL-17 antibody.

As for allergic disease such as asthma; in OVA-sensitized model, the attenuated eosinophilic pulmonary inflammation, the reduced numbers of CD4+ lymphocytes, and the decrease of Th2 cytokines/chemokines level are exhibited in RORγ-KO mice, that is, the allergenic reaction can be inhibited in RORγ-KO mice.

As for dry eye, it is reported that the Th17 cells increases in an animal model of dry eye, and an efficacy of anti-IL-17 antibody in dry eye patient is currently tested in a clinical trial.

As for fibrosis, in a bleomycin-induced pulmonary fibrosis model which is an animal model of pulmonary fibrosis, the administration of anti-IL-17 antibody can inhibit inflammation and fibrosis in lung and can increase survival of the animal.

As for primary biliary cirrhosis, it is reported that Th17 cells in the lesion area of a patient with a primary biliary cirrhosis increase, and an efficacy of an antibody to IL-23 which activates Th17 cells is currently tested in a clinical trial.

As for metabolic disease, the insulin resistance which is induced by feeding a high-fat diet can be suppressed in RORγ KO mice.

On the basis of these findings, RORγ antagonists are thought to be useful for preventing or treating autoimmune disease such as rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica, and type I diabetes; allergic disease such as asthma; dry eye; fibrosis such as pulmonary fibrosis and primary biliary cirrhosis; and metabolic disease such as diabetes.

### SUMMARY OF INVENTION

### Technical Problem

An object of the present invention is to provide novel RORγ antagonists. Another object of the present invention is to provide medicaments of preventing or treating autoimmune disease such as rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica, and type I diabetes; allergic disease such as asthma; dry eye; fibrosis such as pulmonary fibrosis and primary biliary cirrhosis; and metabolic disease such as diabetes.

### Solution to Problem

The present inventors have found triazole compounds which are RORγ antagonists, thereby have completed the present invention. The present inventors have found isoxazole compounds which are RORγ antagonists, thereby have completed the present invention.

That is, the present invention provides the following aspects.

[01] A compound represented by Formula [I]:

or a pharmaceutically acceptable salt thereof, wherein

is monocyclic heteroaromatic group wherein the monocyclic heteroaromatic ring comprises the same or different 1 to 4 hetero atoms selected from nitrogen atom, oxygen atom, or sulfur atom besides carbon atom;
each R^{a1} is the same or different and selected from

(1) C₁₋₆ alkyl group which may optionally be substituted with the same or different 1 to 5 substituents selected from Group A,

(2) halogen atom, or

(3) C₃₋₇ cycloalkyl group which may optionally be substituted with the same or different 1 to 5 substituents selected from Group A;

### R^{b} is

(1) C₁₋₆ alkyl group which may optionally be substituted with the same or different 1 to 5 halogen atoms, or

(2) C₃₋₇ cycloalkyl group;

### R^{c} is

(1) hydrogen atom, or

(2) C₁₋₆ alkyl group;

each R^{d} is the same or different and selected from

(1) halogen atom, or

(2) C₁₋₆ alkyl group which may optionally be substituted with the same or different 1 to 5 halogen atoms;

R^{e} is hydrogen atom;
n^{a} is an integer selected from 0 or 1 to 3;
n^{c} is an integer selected from 0 or 1 to 3;
n^{d} is an integer selected from 0 or 1 to 3;
m is an integer selected from 0 or 1 to 5;

### Group A is

(a) C₁₋₆ alkyl group,

(b) halogen atom,

(c) C₃₋₇ cycloalkyl group which may optionally be substituted with the same or different 1 to 5 substituents selected from the group consisting of C₁₋₆ alkyl group and halogen atom.

[02] The compound according to [01], or a pharmaceutically acceptable salt thereof, wherein

is monocyclic heteroaromatic group selected from the following (1) to (7):

[03] The compound according to [01], or a pharmaceutically acceptable salt thereof, wherein the compound is represented by Formula [II-A]:

wherein each symbol is as defined in [01].
[04] The compound according to [01], or a pharmaceutically acceptable salt thereof, wherein the compound is represented by Formula [II-B]:

wherein each symbol is as defined in [01].
[05] The compound according to [01], or a pharmaceutically acceptable salt thereof, wherein the compound is represented by Formula [II-C]:

wherein each symbol is as defined in [01].
[06] The compound according to [01], or a pharmaceutically acceptable salt thereof, wherein the compound is represented by Formula [II-D]:

wherein each symbol is as defined in [01].
[07] The compound according to [01], or a pharmaceutically acceptable salt thereof, wherein the compound is represented by Formula [II-E]:

wherein each symbol is as defined in [01].
[08] The compound according to [01], or a pharmaceutically acceptable salt thereof, wherein the compound is represented by Formula [II-F]:

wherein each symbol is as defined in [01].
[09] The compound according to [01], or a pharmaceutically acceptable salt thereof, wherein the compound is represented by Formula [II-G]:

wherein each symbol is as defined in [01].
[10] The compound according to any one of [01] to [09], or a pharmaceutically acceptable salt thereof, wherein R^{c} is hydrogen atom.
[11] A pharmaceutical composition comprising the compound according to any one of [01] to [10] or a pharmaceutically acceptable salt thereof, and pharmaceutically acceptable carrier.
[12] A RORγ antagonist comprising the compound according to any one of [01] to [10] or a pharmaceutically acceptable salt thereof.
[13] A medicament for treating or preventing a disease selected from the group consisting of autoimmune disease, allergic disease, dry eye, fibrosis, and metabolic disease, comprising the compound according to any one of [01] to [10] or a pharmaceutically acceptable salt thereof.
[14] The medicament according to [13] wherein the autoimmune disease is selected from the group consisting of rheumatoid arthritis, psoriasis, inflammatory bowel disease, multiple sclerosis, systemic lupus erythematosus, ankylosing spondylitis, uveitis, polymyalgia rheumatica and type I diabetes.
[15] The medicament according to [13] wherein the metabolic disease is diabetes.
[16] A method of inhibiting RORγ in a mammal, comprising administering to said mammal a therapeutically effective amount of the compound according to any one of [01] to [10] or a pharmaceutically acceptable salt thereof.
[17] A method of treating or preventing a disease in a mammal selected from the group consisting of autoimmune disease, allergic disease, dry eye, fibrosis, and metabolic disease, comprising administering to said mammal a therapeutically effective amount of the compound according to any one of [01] to [10] or a pharmaceutically acceptable salt thereof.
[18] The method according to [17] wherein the autoimmune disease is selected from the group consisting of rheumatoid arthritis, psoriasis, inflammatory bowel disease, multiple sclerosis, systemic lupus erythematosus, ankylosing spondylitis, uveitis, polymyalgia rheumatica and type I diabetes.
[19] The method according to [17] wherein the metabolic disease is diabetes.
[20] A pharmaceutical composition for treating or preventing a disease selected from the group consisting of autoimmune disease, allergic disease, dry eye, fibrosis, and metabolic disease, which comprises:

(a) the compound according to any one of [01] to [10] or a pharmaceutically acceptable salt thereof, and

(b) at least one additional medicament for treating or preventing a disease selected from the group consisting of autoimmune disease, allergic disease, dry eye, fibrosis, and metabolic disease.

[21] A combination drug comprising:

(a) the compound according to any one of [01] to [10] or a pharmaceutically acceptable salt thereof, and

(b) at least one additional medicament for treating or preventing a disease selected from the group consisting of autoimmune disease, allergic disease, dry eye, fibrosis, and metabolic disease,

wherein the compound of (a) and the additional medicament of (b) may be administered simultaneously, separately or consecutively.
[22] Use of the compound according to any one of [01] to [10] or a pharmaceutically acceptable salt thereof in the manufacture of a RORγ antagonist.
[23] Use of the compound according to any one of [01] to [10] or a pharmaceutically acceptable salt thereof in the manufacture of a medicament for treating or preventing a disease selected from the group consisting of autoimmune disease, allergic disease, dry eye, fibrosis, and metabolic disease.
[24] The use according to [23] wherein the autoimmune disease is selected from the group consisting of rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica and type I diabetes.
[25] The use according to [24] wherein the metabolic disease is diabetes.
[26] The compound according to any one of [01] to [10] or a pharmaceutically acceptable salt thereof for use in treating or preventing a disease selected from the group consisting of autoimmune disease, allergic disease, dry eye, fibrosis, and metabolic disease.
[27] A commercial package comprising the medicament according to [13], and instructions which explain that the medicament can be used to treat and/or prevent a disease selected from the group consisting of autoimmune disease, allergic disease, dry eye, fibrosis, and metabolic disease.
[28] A commercial package comprising the combination drug according to [21], and instructions which explain that the combination drug can be used to treat and/or prevent a disease selected from the group consisting of autoimmune disease, allergic disease, dry eye, fibrosis, and metabolic disease.
[29] A medicament for treating or preventing a disease selected from the group consisting of autoimmune disease such as rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica and type I diabetes; allergic disease such as asthma; dry eye; fibrosis such as pulmonary fibrosis and primary biliary cirrhosis; and metabolic disease such as diabetes, comprising the compound according to any one of [01] to [10] or a pharmaceutically acceptable salt thereof.
[30] A pharmaceutical composition for treating or preventing a disease selected from the group consisting of autoimmune disease such as rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica and type I diabetes; allergic disease such as asthma; dry eye; fibrosis such as pulmonary fibrosis and primary biliary cirrhosis; and metabolic disease such as diabetes, comprising:

(a) the compound according to any one of [01] to [10] or a pharmaceutically acceptable salt thereof, and

(b) at least one additional medicament for treating or preventing a disease selected from the group consisting of autoimmune disease such as rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica and type I diabetes; allergic disease such as asthma; dry eye; fibrosis such as pulmonary fibrosis and primary biliary cirrhosis; and metabolic disease such as diabetes.

[31] A combination drug comprising:

(a) the compound according to any one of [01] to [10] or a pharmaceutically acceptable salt thereof, and

(b) at least one an additional medicament for treating or preventing a disease selected from the group consisting of autoimmune disease such as rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica and type I diabetes; allergic disease such as asthma; dry eye; fibrosis such as pulmonary fibrosis and primary biliary cirrhosis; and metabolic disease such as diabetes,

wherein the compound of (a) and the additional medicament of (b) may be administered simultaneously, separately or consecutively.
[101] A compound represented by the following formulas:

or a pharmaceutically acceptable salt thereof, wherein

is unsaturated heteromonocyclic group selected from the following (i) to (v):

R^{a} is selected from the following (1) to (12):

(1) C₁₋₁₂ alkyl group which may optionally be substituted with the same or different 1 to 5 substituents selected from Group A,

(2) C₃₋₇ cycloalkyl group which may optionally be substituted with the same or different 1 to 5 substituents selected from Group A,

(3) C₂₋₁₂ alkenyl group which may optionally be substituted with the same or different 1 to 5 substituents selected from Group A,

(4) C₂₋₁₂ alkynyl group which may optionally be substituted with the same or different 1 to 5 substituents selected from Group A,

(5) C₅₋₁₁ spirocyclic cycloalkyl group which may optionally be substituted with the same or different 1 to 5 substituents selected from Group A,

(6) cross-linked C₅₋₁₂ cycloalkyl group which may optionally be substituted with the same or different 1 to 5 substituents selected from Group A,

(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,

(8) phenyl group which may optionally be substituted with the same or different 1 to 5 substituents selected from Group A,

(9) C₉₋₁₀ fused carbocyclic group which may optionally be substituted with the same or different 1 to 5 substituents selected from Group A,

(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,

(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;

R^{b} is selected from the following (1) to (6):

(1) C₁₋₆ alkyl group which may optionally be substituted with the same or different 1 to 5 halogen atoms,

(2) C₃₋₇ cycloalkyl group which may optionally be substituted with the same or different 1 to 5 substituents selected from the group consisting of halogen atom and C₁₋₆ alkyl group,

(3) —CH═CH—C(═O)—OR^{bb1},

(4) —CH₂—CH₂—C(═O)—OR^{bb2},

(5) —CH₂—O—CH₂—C(═O)—OR^{bb3},

(6) hydrogen atom;
R^{bb1}, R^{bb2} and R^{bb3} are each independently hydrogen atom or C₁₋₆ alkyl group;
   each R^{d} is the same or different and selected from the following (1) to (13):

(1) halogen atom,

(2) C₁₋₆ alkyl group which may optionally be substituted with the same or different 1 to 5 substituents selected from Group B,

(3) C₃₋₇ cycloalkyl group which may optionally be substituted with the same or different 1 to 5 substituents selected from Group B,

(4) C₂₋₆ alkenyl group,

(5) cyano group,

(6) —C(═O)—OR^{dd1},

(7) —C(═O)—NR^{dd2}R^{dd3},

(8) —OR^{dd4},

(9) —NR^{dd5}—C(═O)—R^{dd6},

(10) —NR^{dd7}—C(═O)—NR^{dd8}R^{dd9},

(11) —NR^{dd10}—S(═O)₂—R^{dd11},

(12) —NR^{dd12}—S(═O)₂—NR^{dd13}R^{dd14},

(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
   R^{e} is hydrogen atom or C₁₋₆ alkyl group;
   each R^{j} is the same or different C₁₋₁₂ alkyl group which may optionally be substituted with the same or different 1 to 5 substituents selected from Group A;
   Q is selected from the following (1) to (9):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,

(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;

Y is selected from the following (1) to (3):

(1) single bond,

(2) —S(═O)₂—,

(3) C₁₋₃ alkylene which may optionally be substituted with 1 to 3 hydroxyl groups;

m is each independently an integer selected from 0 or 1 to 5;
n^{j} is each independently 0, 1 or 2;
Group A consists of the following (a) to (m):

(a) C₁₋₇ alkyl group which may optionally be substituted with the same or different 1 to 5 substituents selected from Group AA,

(b) halogen atom,

(c) phenyl group,

(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,

(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(g) C₃₋₇ cycloalkyl group which may optionally be substituted with the same or different 1 to 5 substituents selected from Group AA,

(h) —C(═O)—NR^{A1}R^{A2},

(i) —C(═O)—OR^{A3},

(j) —C(═O)—R^{A4},

(k) —OR^{A5},

(l) —NR^{A6}R^{A7},

(m) —S(═O)₂—,
R^{A1}, R^{A2}, R^{A3} and R^{A4} are each independently hydrogen atom or C₁₋₆ alkyl group;
R^{A5}, R^{A6}, and R^{A7} are each independently:
   hydrogen atom,
   C₁₋₆ alkyl group which may optionally be substituted with the same or different 1 to 6 halogen atoms,
   C₃₋₇ cycloalkyl group which may optionally be substituted with the same or different 1 to 5 substituents selected from Group AA,
   benzyl group which may optionally be substituted with the same or different 1 to 5 substituents selected from Group AA,
   phenyl group which may optionally be substituted with the same or different 1 to 5 substituents selected from Group AA,
   C₁₋₆ alkylcarbonyl group,
   C₁₋₆ alkylsulfonyl group, or
   C₂₋₆ alkenyl group; and
R^{A8} is each independently C₁₋₆ alkyl group;
   Group AA consists of the following (a) to (o):

(a) halogen atom,

(b) C₁₋₃ alkyl group which may optionally be substituted with the same or different 1 to 6 halogen atoms,

(c) —OR^{AA1},

(d) —C(═O)—NR^{AA2}R^{AA3},

(e) —C(═O)—OR^{AA4},

(f) —O—C(═O)—R^{AA5},

(g) —C(═O)—R^{AA6},

(h) ═O,

(i) C₃₋₇ cycloalkyl group,

(j) phenyl group which may optionally be substituted with the same or different 1 to 5 C₁₋₃ alkyl groups,

(k) —NR^{AA7}R^{AA8},

(l) —NR^{AA9}—C(═O)—R^{AA10},

(m) —NR^{AA11}—C(═O)—NR^{AA12}R^{AA13},

(n) —NR^{AA14}—S(═O)₂—R^{AA15},

(o) —NR^{AA16}—S(═O)₂—NR^{AA17}R^{AA18};

R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):

(a) halogen atom,

(b) C₃₋₇ cycloalkyl group

(c) —OR^{B1},

(d) —C(═O)—NR^{B2}R^{B3},

(e) —C(═O)—OR^{B4},

(f) C₁₋₆ alkyl group,

(g) —NR^{B5}R^{B6},

(h) —NR^{B7}—C(═O)—R^{B8},

(i) —NR^{B9}—C(═O)—NR^{B10}R^{B11},

(j) —NR^{B12}—S(═O)₂—R^{B13},

(k) —NR^{B14}—S(═O)₂—NR^{B15}R^{B16};

R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently, hydrogen atom or C₁₋₆ alkyl group;
provided that when R^{a} is

(1) phenyl group which may optionally be substituted with the same or different 1 to 5 substituents selected from Group A,

(2) C₉₋₁₀ fused carbocyclic group which may optionally be substituted with the same or different 1 to 5 substituents selected from Group A, or

(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

Q is selected from the following (1) to (6):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered; and further provided that when R^{b} is hydrogen atom,

Q is selected from the following (1) to (5):

(1) phenyl group,

(2) C₃₋₇ cycloalkyl group,

(3) C₉₋₁₀ fused carbocyclic group,

(4) cross-linked C₅₋₁₂ cycloalkyl group,

(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered.

[102] A compound represented by the following formulas:

or a pharmaceutically acceptable salt thereof, wherein

is unsaturated heteromonocyclic group selected from the following (i) to (v):

R^{a} is selected from the following (1) to (12):

(1) C₁₋₁₂ alkyl group which may optionally be substituted with the same or different 1 to 5 substituents selected from Group A,

(2) C₃₋₇ cycloalkyl group which may optionally be substituted with the same or different 1 to 5 substituents selected from Group A,

(3) C₂₋₁₂ alkenyl group which may optionally be substituted with the same or different 1 to 5 substituents selected from Group A,

(4) C₂₋₁₂ alkynyl group which may optionally be substituted with the same or different 1 to 5 substituents selected from Group A,

(5) C₅₋₁₁ spirocyclic cycloalkyl group which may optionally be substituted with the same or different 1 to 5 substituents selected from Group A,

(6) cross-linked C₅₋₁₂ cycloalkyl group which may optionally be substituted with the same or different 1 to 5 substituents selected from Group A,

(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,

(8) phenyl group which may optionally be substituted with the same or different 1 to 5 substituents selected from Group A,

(9) C₉₋₁₀ fused carbocyclic group which may optionally be substituted with the same or different 1 to 5 substituents selected from Group A,

(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,

(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;

R^{b} is selected from the following (1) to (6):

(1) C₁₋₆ alkyl group which may optionally be substituted with the same or different 1 to 5 halogen atoms,

(2) C₃₋₇ cycloalkyl group which may optionally be substituted with the same or different 1 to 5 substituents selected from the group consisting of halogen atom and C₁₋₆ alkyl group,

(3) —CH═CH—C(═O)—OR^{bb1},

(4) —CH₂—CH₂—C(═O)—OR^{bb2},

(5) —CH₂—O—CH₂—C(═O)—OR^{bb3},

(6) hydrogen atom;
R^{bb1}, R^{bb2}, and R^{bb3} are each independently hydrogen atom or C₁₋₆ alkyl group;
   each R^{d} is the same or different and selected from the following (1) to (13):

(1) halogen atom,

(2) C₁₋₆ alkyl group which may optionally be substituted with the same or different 1 to 5 substituents selected from Group B,

(3) C₃₋₇ cycloalkyl group which may optionally be substituted with the same or different 1 to 5 substituents selected from Group B,

(4) C₂₋₆ alkenyl group,

(5) cyano group,

(6) —C(═O)—OR^{dd1},

(7) —C(═O)—NR^{dd2}R^{dd3},

(8) —OR^{dd4},

(9) —NR^{dd5}—C(═O)—R^{dd6},

(10) —NR^{dd7}—C(═O)—NR^{dd8}R^{dd9},

(11) —NR^{dd10}—S(═O)₂—R^{dd11},

(12) —NR^{dd12}—S(═O)₂—NR^{dd13}R^{dd14},

(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
   R^{e} is hydrogen atom or C₁₋₆ alkyl group;
   each R^{j} is the same or different C₁₋₁₂ alkyl group which may optionally be substituted with the same or different 1 to 5 substituents selected from Group A;
   each R^{w} is the same or different and selected from the following (1) to (17):

(1) C₁₋₁₂ alkyl group which may optionally be substituted with the same or different 1 to 5 substituents selected from the group consisting of hydroxyl group and halogen atom,

(2) unsaturated heteromonocyclic group which may optionally be substituted with the same or different 1 to 5 C₁₋₆ alkyl groups wherein the unsaturated heteromonocyclic ring has an unsaturated bond, comprises the same or different 1 to 4 hetero atoms selected from nitrogen atom, oxygen atom, or sulfur atom besides carbon atom, and is 5 to 7-membered,

(3) —(CH₂)_{wn1}—C(═O)—OR^{ww1},

(4) —(CH₂)_{wn1}—C(═O)—(CH₂)_{wn1}—NR^{ww2}R^{ww3},

(5) —(CH₂)_{wn1}—C(═O)—(CH₂)_{wn1}—C(═O)—OR^{ww4},

(6) —NR^{ww5}R^{ww6},

(7) —OR^{ww7},

(8) —C(═O)—R^{ww8},

(9) —S(═O)₂—R^{ww9},

(10) ═O,

(11) methylene,

(12) —(CH₂)_{wn2}-ring P,

(13) halogen atom,

(14) —NR^{ww10}—C(═O)—R^{ww11},

(15) —NR^{ww12}—C(═O)—NR^{ww13}R^{ww14},

(16) —NR^{ww15}—S(═O)₂—R^{ww16},

(17) —NR^{ww17}—S(═O)₂—NR^{ww18}R^{ww19};
R^{ww1}, R^{ww2}, R^{ww3}, R^{ww4}, R^{ww5}, R^{ww6} and R^{ww7} are each independently hydrogen atom or C₁₋₆ alkyl group;
R^{ww8} is each independently
   hydrogen atom,
   C₁₋₆ alkyl group which may optionally be substituted with the same or different 1 to 5 substituents selected from the group consisting of hydroxyl group and halogen atom, or
   saturated heteromonocyclic group which may optionally be substituted with the same or different 1 to 5 alkyl groups wherein the saturated heteromonocyclic ring comprises the same or different 1 to 4 hetero atoms selected from nitrogen atom, oxygen atom, or sulfur atom besides carbon atom, and is 4 to 6-membered;
R^{ww9} is each independently C₁₋₆ alkyl group which may optionally be substituted with the same or different 1 to 5 halogen atoms;
R^{ww10}, R^{ww11}, R^{ww12}, R^{ww13}, R^{ww14}, R^{ww15}, R^{ww16}, R^{ww17}, R^{ww18}, and R^{ww19} are each independently hydrogen atom or C₁₋₆ alkyl group;
ring P is monocyclic heterocycle which is a carboxylic acid equivalent wherein the carboxylic acid equivalent may optionally be substituted;
wn1 is each independently an integer selected from 0 or 1 to 3; and
wn2 is each independently an integer selected from 1 to 3;
   Q is selected from the following (1) to (9):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,

(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;

Y is selected from the following (1) to (3):

(1) single bond,

(2) —S(═O)₂—,

(3) C₁₋₃ alkylene which may optionally be substituted with 1 to 3 hydroxyl groups;

cyclic moiety W is selected from the following (1) to (3):

(1) pyrrolidinyl,

(2) piperidinyl,

(3) C₃₋₇ cycloalkyl;

cn is each independently an integer selected from 0 or 1 to 3;
m is each independently an integer selected from 0 or 1 to 5;
n^{j} is each independently 0, 1 or 2;
Group A consists of the following (a) to (m):

(a) alkyl group which may optionally be substituted with the same or different 1 to 5 substituents selected from Group AA,

(b) halogen atom,

(c) phenyl group,

(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,

(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(g) C₃₋₇ cycloalkyl group which may optionally be substituted with the same or different 1 to 5 substituents selected from Group AA,

(h) —C(═O)—NR^{A1}R^{A2},

(i) —C(═O)—OR^{A3},

(j) —C(═O) —R^{A4},

(k) —OR^{A5},

(l) —NR^{A6}R^{A7},

(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
   hydrogen atom,
   C₁₋₆ alkyl group which may optionally be substituted with the same or different 1 to 6 halogen atoms,
   C₃₋₇ cycloalkyl group which may optionally be substituted with the same or different 1 to 5 substituents selected from Group AA,
   benzyl group which may optionally be substituted with the same or different 1 to 5 substituents selected from Group AA,
   phenyl group which may optionally be substituted with the same or different 1 to 5 substituents selected from Group AA,
   C₁₋₆ alkylcarbonyl group,
   C₁₋₆ alkylsulfonyl group, or
   C₂₋₆ alkenyl group; and
R^{A8} is each independently C₁₋₆ alkyl group;
   Group AA consists of the following (a) to (o):

(a) halogen atom,

(b) C₁₋₃ alkyl group which may optionally be substituted with the same or different 1 to 6 halogen atoms,

(c) —OR^{AA1},

(d) —C(═O)—NR^{AA2}R^{AA3},

(e) —C(═O)—OR^{AA4},

(f) —O—C(═O)—R^{AA5},

(g) —C(═O)—R^{AA6},

(h) ═O,

(i) C₃₋₇ cycloalkyl group,

(j) phenyl group which may optionally be substituted with the same or different 1 to 5 C₁₋₃ alkyl groups,

(k) —NR^{AA7}R^{AA8},

(l) —NR^{AA9}—C(═O)—R^{AA10},

(m) —NR^{AA11}—C(═O)—NR^{AA12}R^{AA13},

(n) —NR^{AA14}—S(═O)₂—R^{AA15},

(o) —NR^{AA16}—S(═O)₂—NR^{AA17}R^{AA18},

R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):

(a) halogen atom,

(b) C₃₋₇ cycloalkyl group

(c) —OR^{B1},

(d) —C(═O)—NR^{B2}R^{B3},

(e) —C(═O)—OR^{B4},

(f) C₁₋₆ alkyl group,

(g) —NR^{B5}R^{B6},

(h) —NR^{B7}—C(═O)—R^{B8},

(i) —NR^{B8}—C(═O)—NR^{B10}R^{B11},

(j) —NR^{B12}—S(═O)₂—R^{B13},

(k) —NR^{B14}—S(═O)₂—NR^{B15}R^{B16};

R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently, hydrogen atom or C₁₋₆ alkyl group;
provided that when R^{b} is hydrogen atom,
Q is selected from the following (1) to (5):

(1) phenyl group,

(2) C₃₋₇ cycloalkyl group,

(3) C₉₋₁₅ fused carbocyclic group,

(4) cross-linked C₅₋₁₂ cycloalkyl group,

(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;

and further provided that when

is

cyclic moiety W is selected from the following (1) or (2):

(1) pyrrolidinyl,

(2) piperidinyl.

[103] A compound represented by the following formulas:

or a pharmaceutically acceptable salt thereof, wherein

is unsaturated heteromonocyclic group selected from the following (i) to (v):

R^{a} is selected from the following (1) to (12):

(1) C₁₋₁₂ alkyl group which may optionally be substituted with the same or different 1 to 5 substituents selected from Group A,

(2) C₃₋₇ cycloalkyl group which may optionally be substituted with the same or different 1 to 5 substituents selected from Group A,

(3) C₂₋₁₂ alkenyl group which may optionally be substituted with the same or different 1 to 5 substituents selected from Group A,

(4) C₂₋₁₂ alkynyl group which may optionally be substituted with the same or different 1 to 5 substituents selected from Group A,

(5) C₅₋₁₁ spirocyclic cycloalkyl group which may optionally be substituted with the same or different 1 to 5 substituents selected from Group A,

(6) cross-linked C₅₋₁₂ cycloalkyl group which may optionally be substituted with the same or different 1 to 5 substituents selected from Group A,

(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,

(8) phenyl group which may optionally be substituted with the same or different 1 to 5 substituents selected from Group A,

(9) C₉₋₁₀ fused carbocyclic group which may optionally be substituted with the same or different 1 to 5 substituents selected from Group A,

(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,

(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;

R^{b} is selected from the following (1) to (6):

(1) C₁₋₆ alkyl group which may optionally be substituted with the same or different 1 to 5 halogen atoms,

(2) C₃₋₇ cycloalkyl group which may optionally be substituted with the same or different 1 to 5 substituents selected from the group consisting of halogen atom and C₁₋₆ alkyl group,

(3) —CH═CH—C(═O)—OR^{bb1},

(4) —CH₂—CH₂—C(═O)—OR^{bb2},

(5) —CH₂—O—CH₂—C(═O)—OR^{bb3},

(6) hydrogen atom;
R^{bb1}, R^{bb2}, and R^{bb3} are each independently hydrogen atom or C₁₋₆ alkyl group;
   R^{c} is selected from the following (1) to (17):

(1) —(CH₂)ₙ₁—C(═O)—OR^{cc1},

(2) —O—(CH₂)ₙ₂—C(═O)—OR^{cc2},

(3) —(CH₂)ₙ₃-ring P,

(4) —(CH₂)ₙ₄—C(═O)—NH—S(═O)₂—CH₃,

(5) C₁₋₆ alkyl group which may optionally be substituted with the same or different 1 to 5 substituents selected from Group 0,

(6) C₃₋₆ cycloalkyl group which may optionally be substituted with the same or different 1 to 5 substituents selected from Group C,

(7) C₂₋₁₂ alkenyl group,

(8) C₂₋₁₂ alkynyl group,

(9) —NR^{cc3}R^{cc4},

(10) —OR^{cc5},

(11) —O—CH₂CH₂—OH,

(12) —O—CH₂C(═O)NH—CH₃,

R^{cc1} and R^{cc2} are each independently hydrogen atom or C₁₋₆ alkyl group;
R^{cc3} and R^{cc4} are each independently, hydrogen atom,
C₁₋₆ alkyl group,
C₁₋₆ alkylcarbonyl group,
benzyloxycarbonyl group, or
C₁₋₆ alkyl group which is substituted with 1 to 3 hydroxyl groups;
R^{cc5} is
   hydrogen atom,
   C₁₋₆ alkyl group which may optionally be substituted with the same or different 1 to 5 substituents selected from Group C, or
   benzyl group;
ring P is monocyclic heterocycle which is a carboxylic acid equivalent wherein the carboxylic acid equivalent may optionally be substituted;
n1 is an integer selected from 0 or 1 to 3;
n2 is an integer selected from 0 or 1 to 3;
n3 is an integer selected from 1 to 3; and
n4 is an integer selected from 1 to 3;
   each R^{d} is the same or different and selected from the following (1) to (13):

(1) halogen atom,

(2) C₁₋₆ alkyl group which may optionally be substituted with the same or different 1 to 5 substituents selected from Group B,

(3) C₃₋₇ cycloalkyl group which may optionally be substituted with the same or different 1 to 5 substituents selected from Group B,

(4) C₂₋₆ alkenyl group,

(5) cyano group,

(6) —C(═O)—OR^{dd1},

(7) —C(═O)—NR^{dd2}R^{dd3},

(8) —OR^{dd4},

(9) —NR^{dd5}—C(═O)—R^{dd6},

(10) —NR^{dd7}—C(═O)—NR^{dd8}R^{dd9},

(11) —NR^{dd10}—S(═O)₂—R^{dd11},

(12) —NR^{dd12}—S(═O)₂—NR^{dd13}R^{dd14},

(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group. B;
   R^{e} is hydrogen atom or C₁₋₆ alkyl group;
   each R^{j} is the same or different C₁₋₁₂ alkyl group which may optionally be substituted with the same or different 1 to 5 substituents selected from Group A;
   Q is selected from the following (1) to (9):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,

(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;

Y is selected from the following (1) to (3):

(1) single bond,

(2) —S(═O)₂—,

(3) C₁₋₃ alkylene which may optionally be substituted with 1 to 3 hydroxyl groups;

m is each independently an integer selected from 0 or 1 to 5;
n^{j} is each independently 0, 1 or
Group A consists of the following (a) to (m):

(a) C₁₋₇ alkyl group which may optionally be substituted with the same or different 1 to 5 substituents selected from Group AA,

(b) halogen atom,

(c) phenyl group,

(d) saturated heteromonocyclic group which may optionally substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,

(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(g) C₃₋₇ cycloalkyl group which may optionally be substituted with the same or different 1 to 5 substituents selected from Group AA,

(h) —C(═O)—NR^{A1}R^{A2},

(i) —C(═O)—OR^{A3},

(j) —C(═O)—R^{A4},

(k) —OR^{A5},

(l) —NR^{A6}R^{A7},

(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
   hydrogen atom,
   C₁₋₆ alkyl group which may optionally be substituted with the same or different 1 to 6 halogen atoms,
   C₃₋₇ cycloalkyl group which may optionally be substituted with the same or different 1 to 5 substituents selected from Group AA,
   benzyl group which may optionally be substituted with the same or different 1 to 5 substituents selected from Group AA,
   phenyl group which may optionally be substituted with the same or different 1 to 5 substituents selected from Group AA,
   C₁₋₆ alkylcarbonyl group,
   C₁₋₆ alkylsulfonyl group, or
   C₂₋₆ alkenyl group; and
R^{A8} is each independently C₁₋₆ alkyl group;
   Group AA consists of the following (a) to (o):

(a) halogen atom,

(b) C₁₋₃ alkyl group which may optionally be substituted with the same or different 1 to 6 halogen atoms,

(c) —OR^{AA1},

(d) —C(═O)—NR^{AA2}R^{AA3},

(e) —C(═O)—OR^{AA4},

(f) —O—C(═O)—R^{AA5},

(g) —C(═O)—R^{AA6},

(h) ═O,

(i) C₃₋₇ cycloalkyl group,

(j) phenyl group which may optionally be substituted with the same or different 1 to 5 C₁₋₃ alkyl groups,

(k) —NR^{AA7}R^{AA8},

(l) —NR^{AA9}—C(═O)—R^{AA10},

(m) —NR^{AA11}—(═O)—NR^{AA12}R^{AA13},

(n) —NR^{AA14}—S(═O)₂—R^{AA15},

(o) —NR^{AA16}—S(═O)₂—NR^{AA17}R^{AA18};

R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):

(a) halogen atom,

(b) C₃₋₇ cycloalkyl group

(c) —OR^{B1},

(d) —C(═O)—NR^{B2}R^{B3},

(e) —C(═O)—OR^{B4},

(f) C₁₋₆ alkyl group,

(g) —NR^{B5}R^{B6},

(h) —NR^{B7}—C(═O)—R^{B8},

(i) —NR^{B9}—C(═O)—NR^{B10}R^{B11},

(j) —NR^{B12}—S—(═O)₂—R^{B13};

(k) —NR^{B14}—S(═O)₂—NR^{B15}R^{B16};
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently hydrogen atom or C₁₋₆ alkyl group;
   Group C consists of the following (a) to (k):

(a) phenyl group which may optionally be substituted with the same or different 1 to 5 substituents selected from Group CC,

(b) cyano group,

(c) halogen atom,

(d) —OR^{CD1},

(e) —NR^{CD2}R^{CD3},

(f) —C(═O)—NR^{CD4}R^{CD5},

(i) saturated heteromonocyclic group which may optionally be substituted with the same or different 1 to 5 substituents selected from Group CC wherein the saturated heteromonocyclic ring comprises the same or different 1 to 4 hetero atoms selected from nitrogen atom, oxygen atom, or sulfur atom besides carbon atom, and is 4 to 6-membered, excluding ring P,

(j) unsaturated heteromonocyclic group which may optionally be substituted with the same or different 1 to 5 substituents selected from Group CC wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered, excluding ring P,

R^{CD1}, R^{CD2}, and R^{CD3} are each independently,
   hydrogen atom,
   C₁₋₆ alkyl group,
   C₁₋₆ alkylcarbonyl group,
   benzyl group,
   —C(═O)—O—CH₂-phenyl,
   —C(═O)—N(CH₃)₂,
   —C(═O)—C(OH) (CH₃)₂,
   —C(═O)—CH₂—O—CH₃, or
   —C(═O)—CH₂—CN; and
R^{CD4} and R^{CD5} are each independently hydrogen atom or C₁₋₆ alkyl group;
   Group CC consists of the following (a) to (c):

(a) halogen atom,

(b) C₁₋₃ alkyl group which may optionally be substituted with the same or different 1 to 6 halogen atoms,

(c) —OR^{AA1},
R^{AA1} is each independently hydrogen atom or C₁₋₆ alkyl group;
   provided that when R^{a} is

(1) phenyl group which may optionally be substituted with the same or different 1 to 5 substituents selected from Group A,

(2) C₉₋₁₀ fused carbocyclic group which may optionally be substituted with the same or different 1 to 5 substituents selected from Group A, or

(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

Q is selected from the following (1) to (6):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;

and further provided that when R^{b} is hydrogen atom,
Q is selected from the following (1) to (5):

(1) phenyl group,

(2) C₃₋₇ cycloalkyl group,

(3) C₉₋₁₀ fused carbocyclic group,

(4) cross-linked C₅₋₁₂ cycloalkyl group,

(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered.

[104] A compound represented by the following formulas:

or a pharmaceutically acceptable salt thereof, wherein

is unsaturated heteromonocyclic group selected from the following (i) to (v):

R^{a} is selected from the following (1) to (12):

(1) C₁₋₁₂ alkyl group which may optionally be substituted with the same or different 1 to 5 substituents selected from Group A,

(2) C₃₋₇ cycloalkyl group which may optionally be substituted with the same or different 1 to 5 substituents selected from Group A,

(3) C₂₋₁₂ alkenyl group which may optionally be substituted with the same or different 1 to 5 substituents selected from Group A,

(4) C₂₋₁₂ alkynyl group which may optionally be substituted with the same or different 1 to 5 substituents selected from Group A,

(5) C₅₋₁₁ spirocyclic cycloalkyl group which may optionally be substituted with the same or different 1 to 5 substituents selected from Group A,

(6) cross-linked C₅₋₁₂ cycloalkyl group which may optionally be substituted with the same or different 1 to 5 substituents selected from Group A,

(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,

(8) phenyl group which may optionally be substituted with the same or different 1 to 5 substituents selected from Group A,

(9) C₉₋₁₀ fused carbocyclic group which may optionally be substituted with the same or different 1 to 5 substituents selected from Group A,

(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,

(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;

R^{b} is selected from the following (1) to (6):

(1) C₁₋₆ alkyl group which may optionally be substituted with the same or different 1 to 5 halogen atoms,

(2) C₃₋₇ cycloalkyl group which may optionally be substituted with the same or different 1 to 5 substituents selected from the group consisting of halogen atom and C₁₋₆ alkyl group,

(3) —CH═CH—C(═O)—OR^{bb1},

(4) —CH₂—CH₂—C(═O)—OR^{bb2},

(5) —CH₂—O—CH₂—C(═O)—OR^{bb3},

(6) hydrogen atom;
R^{bb1}, R^{bb2} and R^{bb3} are each independently hydrogen atom or C₁₋₆ alkyl group;
   R^{c} is selected from the following (1) to (17):

(1) —(CH₂)ₙ₁—C(═O)—OR^{cc1},

(2) —O—(CH₂)ₙ₂—C(═O)—OR^{cc2},

(3) —(CH₂)ₙ₃-ring P,

(4) —(CH₂)ₙ₄—C(═O)—NH—S(═O)₂—CH₃,

(5) C₁₋₆ alkyl group which may optionally be substituted with the same or different 1 to 5 substituents selected from Group C,

(6) C₃₋₆ cycloalkyl group which may optionally be substituted with the same or different 1 to 5 substituents selected from Group C,

(7) C₂₋₁₂ alkenyl group,

(8) C₂₋₁₂ alkynyl group,

(9) —NR^{cc3}R^{cc4},

(10) —OR^{cc5},

(11) —O—CH₂CH₂—OH,

(12) —O—CH₂O(═O)NH—CH₃,

R^{cc1} and R^{cc2} are each independently hydrogen atom or C₁₋₆ alkyl group;
R^{cc3} and R^{cc4} are each independently,
hydrogen atom,
C₁₋₆ alkyl group,
C₁₋₆ alkylcarbonyl group,
benzyloxycarbonyl group, or
C₁₋₆ alkyl group which is substituted with 1 to 3 hydroxyl groups;
R^{cc5} is
   hydrogen atom,
   C₁₋₆ alkyl group which may optionally be substituted with the same or different 1 to 5 substituents selected from Group C, or
   benzyl group;
ring P is monocyclic heterocycle which is a carboxylic acid equivalent wherein the carboxylic acid equivalent may optionally be substituted;
n1 is an integer selected from 0 or 1 to 3;
n2 is an integer selected from 0 or 1 to 3;
n3 is an integer selected from 1 to 3; and
n4 is an integer selected from 1 to 3;
   each R^{d} is the same or different and selected from the following (1) to (13):

(1) halogen atom,

(2) C₁₋₆ alkyl group which may optionally be substituted with the same or different 1 to 5 substituents selected from Group B,

(3) C₃₋₇ cycloalkyl group which may optionally be substituted with the same or different 1 to 5 substituents selected from Group B,

(4) C₂₋₆ alkenyl group,

(5) cyano group,

(6) —C(═O)—OR^{dd1},

(7) —C(═O)—NR^{dd2}R^{dd3},

(8) —OR^{dd4},

(9) —NR^{dd5}—C(═O)—R^{dd6},

(10) —NR^{dd7}—C(═O)—NR^{dd8}R^{dd9},

(11) —NR^{dd10}—S(═O)₂—R^{dd11},

(12) —NR^{dd12}—S(═O)₂—NR^{dd13}—R^{dd14},

(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
   R^{e} is hydrogen atom or C₁₋₆ alkyl group;
   R^{f} is hydrogen atom or C₁₋₆ alkyl group;
   each R^{j} is the same or different C₁₋₁₂ alkyl group which may optionally be substituted with the same or different 1 to 5 substituents selected from Group A;
   Q is selected from the following (1) to (9):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,

(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;

Y is selected from the following (1) to (3):

(1) single bond,

(2) —S(═O)₂—,

(3) C₁₋₃ alkylene which may optionally be substituted with 1 to 3 hydroxyl groups;

m is each independently an integer selected from 0 or 1 to 5;
n^{j} is each independently 0, 1 or 2;
Group A consists of the following (a) to (m):

(a) alkyl group which may optionally be substituted with the same or different 1 to 5 substituents selected from Group AA,

(b) halogen atom,

(c) phenyl group,

(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,

(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(g) C₃₋₇ cycloalkyl group which may optionally be substituted with the same or different 1 to 5 substituents selected from Group AA,

(h) —C(═O)—NR^{A2}R^{A2},

(i) —C(═O)—OR^{A5},

(j) —C(═O)—R^{A4},

(k) —OR^{A5},

(l) —NR^{A6}R^{A7},

(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
   hydrogen atom,
   C₁₋₆ alkyl group which may optionally be substituted with the same or different 1 to 6 halogen atoms,
   C₃₋₇ cycloalkyl group which may optionally be substituted with the same or different 1 to 5 substituents selected from Group AA,
   benzyl group which may optionally be substituted with the same or different 1 to 5 substituents selected from Group AA,
   phenyl group which may optionally be substituted with the same or different 1 to 5 substituents selected from Group AA,
   C₁₋₆ alkylcarbonyl group,
   C₁₋₆ alkylsulfonyl group, or
   C₂₋₆ alkenyl group; and
R^{A8} is each independently C₁₋₆ alkyl group;
   Group AA consists of the following (a) to (o):

(a) halogen atom,

(b) C₁₋₃ alkyl group which may optionally be substituted with the same or different 1 to 6 halogen atoms,

(c) —OR^{AA1},

(d) —C(═O)—NR^{AA2}R^{AA3},

(e) —C(═O)—OR^{AA4},

(f) —O—C(═O)—R^{AA5},

(g) —C(═O)—R^{AA6},

(h) ═O,

(i) C₃₋₇ cycloalkyl group,

(j) phenyl group which may optionally be substituted with the same or different 1 to 5 C₁₋₃ alkyl groups,

(k) —NR^{AA7}R^{AA8},

(l) —NR^{AA9}—C(═O)—R^{AA10},

(m) —NR^{AA11}—C(═O)—NR^{AA12}R^{AA13},

(n) —NR^{AA14}—S(═O)₂—R^{AA15},

(o) —NR^{AA16}—S(═)₂—NR^{AA17}R^{AA18};

R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):

(a) halogen atom,

(b) C₃₋₇ cycloalkyl group

(c) —OR^{B1},

(d) —C(═O)—NR^{B2}R^{B3},

(e) —C(═O)—OR^{B4},

(f) C₁₋₆ alkyl group,

(g) —NR^{B5}R^{B6},

(h) —NR^{B7}—C(═O)—R^{B9},

(i) —NR^{B9}—C(═O)—NR^{B10}R^{B11},

(j) —NR^{B12}—S(═O)₂—R^{B13},

(k) —NR^{B14}—S(═O)₂—NR^{B15}R^{B16};
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently hydrogen atom or C₁₋₆ alkyl group;
   Group C consists of the following (a) to (k):

(a) phenyl group which may optionally be substituted with the same or different 1 to 5 substituents selected from Group CC,

(b) cyano group,

(c) halogen atom,

(d) —OR^{CD1},

(e) —NR^{CD2}R^{CD3},

(f) —C(═O)—NR^{CD4}R^{CD5},

(i) saturated heteromonocyclic group which may optionally be substituted with the same or different 1 to 5 substituents selected from Group CC wherein the saturated heteromonocyclic ring comprises the same or different 1 to 4 hetero atoms selected from nitrogen atom, oxygen atom, or sulfur atom besides carbon atom, and is 4 to 6-membered, excluding ring P,

(j) unsaturated heteromonocyclic group which may optionally be substituted with the same or different 1 to 5 substituents selected from Group CC wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered, excluding ring P,

R^{CD1}, R^{CD2}, and R^{CD3} are each independently,
   hydrogen atom,
   C₁₋₆ alkyl group,
   C₁₋₆ alkylcarbonyl group,
   benzyl group,
   —C(═O)—O—CH₂-phenyl,
   —C(═O)—N(CH₃)₂,
   —C(═O)—C(OH) (CH₃)₂,
   —C(═O)—CH₂—O—CH₃, or
   —C(═O)—CH₂—CN; and
R^{CD4} and R^{CD5} are each independently hydrogen atom or C₁₋₆ alkyl group;
   Group CC consists of the following (a) to (c):

(a) halogen atom,

(b) C₁₋₃ alkyl group which may optionally be substituted with the same or different 1 to 6 halogen atoms,

(c) —OR^{AA1},
R^{AA1} is each independently hydrogen atom or C₁₋₆ alkyl group;
   provided that when R^{a} is

(1) phenyl group which may optionally be substituted with the same or different 1 to 5 substituents selected from Group A,

(2) C₉₋₁₀ fused carbocyclic group which may optionally be substituted with the same or different 1 to 5 substituents selected from Group A, or

(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

Q is selected from the following (1) to (6):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;

further provided that when R^{b} is hydrogen atom,
Q is selected from the following (1) to (5):

(1) phenyl group,

(2) C₃₋₇ cycloalkyl group,

(3) C₉₋₁₀ fused carbocyclic group,

(4) cross-linked C₅₋₁₂ cycloalkyl group,

(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;

and further provided that when

is

Q is selected from the following (1) to (6):

(1) C₃₋₇ cycloalkyl group,

(2) C₉₋₁₀ fused carbocyclic group selected from the group consisting of indanyl group and 1,2,3,4-tetrahydronaphthyl group,

(3) cross-linked C₅₋₁₂ cycloalkyl group,

(4) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,

(5) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(6) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered.

[105] A compound represented by the following formulas:

or a pharmaceutically acceptable salt thereof, wherein

is unsaturated heteromonocyclic group selected from the following (i) to (v):

R^{a} is selected from the following (1) to (12):

(1) C₁₋₁₂ alkyl group which may optionally be substituted with the same or different 1 to 5 substituents selected from Group A,

(2) C₃₋₇ cycloalkyl group which may optionally be substituted with the same or different 1 to 5 substituents selected from Group A,

(3) C₂₋₁₂ alkenyl group which may optionally be substituted with the same or different 1 to 5 substituents selected from Group A,

(4) C₂₋₁₂ alkynyl group which may optionally be substituted with the same or different 1 to 5 substituents selected from Group A,

(5) C₅₋₁₁ spirocyclic cycloalkyl group which may optionally be substituted with the same or different 1 to 5 substituents selected from Group A,

(6) cross-linked C₅₋₁₂ cycloalkyl group which may optionally be substituted with the same or different 1 to 5 substituents selected from Group A,

(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,

(8) phenyl group which may optionally be substituted with the same or different 1 to 5 substituents selected from Group A,

(9) C₉₋₁₀ fused carbocyclic group which may optionally be substituted with the same or different 1 to 5 substituents selected from Group A,

(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,

(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;

R^{b} is selected from the following (1) to (6):

(1) C₁₋₆ alkyl group which may optionally be substituted with the same or different 1 to 5 halogen atoms,

(2) C₃₋₇ cycloalkyl group which may optionally be substituted with the same or different 1 to 5 substituents selected from the group consisting of halogen atom and C₁₋₆ alkyl group,

(3) —CH═CH—C(═O)—OR^{bb1},

(4) —CH₂—CH₂—C(═O)—OR^{bb2},

(5) —CH₂—O—CH₂—C(═O)—OR^{bb3},

(6) hydrogen atom;
R^{bb1}, R^{bb2}, and R^{bb3} are each independently hydrogen atom or C₁₋₆ alkyl group;
   R^{c} is selected from the following (1) to (17):

(1) —(CH₂)ₙ₁—C(═O)—OR^{cc1},

(2) —O—(CH₂)ₙ₂—C(═O)—OR^{cc2},

(3) —(CH₂)ₙ₃-ring P,

(4) —(CH₂)ₙ₄—C(═O)—NH—S(═O)₂—CH₃,

(5) C₁₋₆ alkyl group which may optionally be substituted with the same or different 1 to 5 substituents selected from Group C,

(6) C₃₋₆ cycloalkyl group which may optionally be substituted with the same or different 1 to 5 substituents selected from Group C,

(7) C₂₋₁₂ alkenyl group,

(8) C₂₋₁₂ alkynyl group,

(9) —NR^{cc3}R^{cc4},

(10) —OR^{cc5},

(11) —O—CH₂CH₂—OH,

(12) —O—CH₂C(═O)NH—CH₃,

R^{cc1} and R^{cc2} are each independently hydrogen atom or C₁₋₆ alkyl group;
R^{cc3} and R^{cc4} are each independently,
hydrogen atom,
C₁₋₆ alkyl group,
C₁₋₆ alkylcarbonyl group,
benzyloxycarbonyl group, or
C₁₋₆ alkyl group which is substituted with 1 to 3 hydroxyl groups;
R^{cc5} is
   hydrogen atom,
   C₁₋₆ alkyl group which may optionally be substituted with the same or different 1 to 5 substituents selected from Group C, or
   benzyl group;
ring P is monocyclic heterocycle which is a carboxylic acid equivalent wherein the carboxylic acid equivalent may optionally be substituted;
n1 is an integer selected from 0 or 1 to 3;
n2 is an integer selected from 0 or 1 to 3;
n3 is an integer selected from 1 to 3; and
n4 is an integer selected from 1 to 3;
   each R^{d} is the same or different and selected from the following (1) to (13)

(1) halogen atom,

(2) C₁₋₆ alkyl group which may optionally be substituted with the same or different 1 to 5 substituents selected from Group B,

(3) C₃₋₇ cycloalkyl group which may optionally be substituted with the same or different 1 to 5 substituents selected from Group B,

(4) C₂₋₆ alkenyl group,

(5) cyano group,

(6) —C(═O)—OR^{dd1},

(7) —C(═O)—NR^{dd2}R^{dd3},

(8) —OR^{dd4},

(9) —NR^{dd5}—C(═O)—R^{dd6},

(10) —NR^{dd7}—C(═O)—NR^{dd8}R^{dd9},

(11) —NR^{dd10}—S(═O)₂—R^{dd11},

(12) —NR^{dd12}—S(═O)₂—NR^{dd13}R^{dd14},

(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or alkyl group which may optionally be substituted with the same or different 1 to 5 substituents selected from Group B;
   R^{e} is hydrogen atom or C₁₋₆ alkyl group;
   R^{f} is hydrogen atom or C₁₋₆ alkyl group;
   each R^{j} is the same or different C₁₋₁₂ alkyl group which may optionally be substituted with the same or different 1 to 5 substituents selected from Group A;
   Q is selected from the following (1) to (9):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₉ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,

(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered, unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;

Y is selected from the following (1) to (3):

(1) single bond,

(2) —S(═O)₂—,

(3) C₁₋₃ alkylene which may optionally be substituted with 1 to 3 hydroxyl groups;

m is each independently an integer selected from 0 or 1 to 5;
n^{j} is each independently 0, 1 or 2;
Group A consists of the following (a) to (m):

(a) C₁₋₇ alkyl group which may optionally be substituted with the same or different 1 to 5 substituents selected from Group AA,

(b) halogen atom,

(c) phenyl group,

(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,

(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(g) C₃₋₇ cycloalkyl group which may optionally be substituted with the same or different 1 to 5 substituents selected from Group AA,

(h) —C(═O)—NR^{A1}R^{A2},

(i) —C(═O)—OR^{A3},

(j) —C(═O)—R^{A4},

(k) —OR^{A5},

(l) —NR^{A6}R^{A7},

(m) —S(═O)₂R^{A8};
R^{A1}, R^{A2}, R^{A3}, and R^{A4} are each independently hydrogen atom or C₁₋₆ alkyl group;
R^{A5}, R^{A6}, and R^{A7} are each independently:
   hydrogen atom,
   C₁₋₆ alkyl group which may optionally be substituted with the same or different 1 to 6 halogen atoms,
   C₃₋₇ cycloalkyl group which may optionally be substituted with the same or different 1 to 5 substituents selected from Group AA,
   benzyl group which may optionally be substituted with the same or different 1 to 5 substituents selected from Group AA,
   phenyl group which may optionally be substituted with the same or different 1 to 5 substituents selected from Group AA,
      C₁₋₆ alkylcarbonyl group,
      C₁₋₆ alkylsulfonyl group, or
      C₂₋₆ alkenyl group; and
   R^{A8} is each independently C₁₋₆ alkyl group;
      Group AA consists of the following (a) to (o):

(a) halogen atom,

(b) C₁₋₃ alkyl group which may optionally be substituted with the same or different 1 to 6 halogen atoms,

(c) —OR^{AA1},

(d) —C(═O)—NR^{AA2}R^{AA3},

(e) —C(═O)—OR^{AA4},

(f) —O—C(═O)—R^{AA5},

(g) —C(═O)—R^{AA6},

(h) ═O,

(i) C₃₋₇ cycloalkyl group,

(j) phenyl group which may optionally be substituted with the same or different 1 to 5 C₁₋₃ alkyl groups,

(k) —NR^{AA7}R^{AA8},

(l) —NR^{AA9}—C(═O)—R^{AA10},

(m) —NR^{AA11}—C(═O)—NR^{AA12}R^{AA13},

(n) —NR^{AA14}—S(═O)₂—R^{AA15},

(o) —NR^{AA16}—S(═O)₂—NR^{AA17}R^{AA18};

R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):

(a) halogen atom,

(b) C₃₋₇ cycloalkyl group

(c) —OR^{B1},

(d) —C(═O)—NR^{B2}R^{B3},

(e) —C(═O)—OR^{B4},

(f) C₁₋₆ alkyl group,

(g) —NR^{B5}R^{B6},

(h) —NR^{B7}—C(═O)—R^{B8},

(i) —NR^{B9}—C(═O)—NR^{B10}R^{B11},

(j) —NR^{B12}—S(═O)₂—R^{B13},

(k) —NR^{B14}—S(═O)₂—NR^{B15}R^{B16};
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently hydrogen atom or C₁₋₆ alkyl group;
   Group C consists of the following (a) to (k):

(a) phenyl group which may optionally be substituted with the same or different 1 to 5 substituents selected from Group CC,

(b) cyano group,

(c) halogen atom,

(d) —OR^{CD1},

(e) —NR^{CD2}R^{CD3},

(f) —C(═O)—NR^{CD4}R^{CD5},

(i) saturated heteromonocyclic group which may optionally be substituted with the same or different 1 to 5 substituents selected from Group CC wherein the saturated heteromonocyclic ring comprises the same or different 1 to 4 hetero atoms selected from nitrogen atom, oxygen atom, or sulfur atom besides carbon atom, and is 4 to 6-membered, excluding ring P,

(j) unsaturated heteromonocyclic group which may optionally be substituted with the same or different 1 to 5 substituents selected from Group CC wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered, excluding ring P,

R^{CD1}, R^{CD2}, and R^{CD3} are each independently,
   hydrogen atom,
   C₁₋₆ alkyl group,
   C₁₋₆ alkylcarbonyl group,
   benzyl group,
   —C(═O)—O—CH₂-phenyl,
   —C(═O)—N(CH₂)₂,
   —C(═O)—C(OH) (CH₃)₂,
   —C(═O)—CH₂—O—CH₃, or
   —C(═O)—CH₂—CN; and
R^{CD4} and R^{CD5} are each independently hydrogen atom or C₁₋₆ alkyl group;
   Group CC consists of the following (a) to (c):

(a) halogen atom,

(b) C₁₋₃ alkyl group which may optionally be substituted with the same or different 1 to 6 halogen atoms,

(c) —OR^{AA1},
R^{AA1} is each independently hydrogen atom or C₁₋₆ alkyl group;
   provided that when R^{a} is

(1) phenyl group which may optionally be substituted with the same or different 1 to 5 substituents selected from Group A,

(2) C₉₋₁₀ fused carbocyclic group which may optionally be substituted with the same or different 1 to 5 substituents selected from Group A, or

(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

Q is selected from the following (1) to (6):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;

further provided that when R^{b} is hydrogen atom,
Q is selected from the following (1) to (5):

(1) phenyl group,

(2) C₃₋₇ cycloalkyl group,

(3) C₉₋₁₀ fused carbocyclic group,

(4) cross-linked C₃₋₁₂ cycloalkyl group,

(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered and further provided that when

is

R^{a} is selected from the following (1) or (2):

(1) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,

(2) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered.

[106] A compound represented by the following formulas:

or a pharmaceutically acceptable salt thereof, wherein

is unsaturated heteromonocyclic group selected from the following (i) to (v):

R^{a} is selected from the following (1) to (12):

(1) C₁₋₁₂ alkyl group which may optionally be substituted with the same or different 1 to 5 substituents selected from Group A,

(2) C₃₋₇ cycloalkyl group which may optionally be substituted with the same or different 1 to 5 substituents selected from Group A,

(3) C₂₋₁₂ alkenyl group which may optionally be substituted with the same or different 1 to 5 substituents selected from Group A,

(4) C₂₋₁₂ alkynyl group which may optionally be substituted with the same or different 1 to 5 substituents selected from Group A,

(5) C₅₋₁₁ spirocyclic cycloalkyl group which may optionally be substituted with the same or different 1 to 5 substituents selected from Group A,

(6) cross-linked C₅₋₁₂ cycloalkyl group which may optionally be substituted with the same or different 1 to 5 substituents selected from Group A,

(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,

(8) phenyl group which may optionally be substituted with the same or different 1 to 5 substituents selected from Group A,

(9) C₉₋₁₀ fused carbocyclic group which may optionally be substituted with the same or different 1 to 5 substituents selected from Group A,

(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,

(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;

R^{b} is selected from the following (1) to (6):

(1) C₁₋₆ alkyl group which may optionally be substituted with the same or different 1 to 5 halogen atoms,

(2) C₃₋₇ cycloalkyl group which may optionally be substituted with the same or different 1 to 5 substituents selected from the group consisting of halogen atom and C₁₋₆ alkyl group,

(3) —CH═CH—C(═O)—OR^{bb1},

(4) —CH₂—CH₂—C(═O)—OR^{bb2},

(5) —CH₂—O—CH₂—C(═O)—OR^{bb3},

(6) hydrogen atom;
R^{bb1}, R^{bb2}, and R^{bb3} are each independently hydrogen atom or C₁₋₆ alkyl group;
   R^{c} is selected from the following (1) to (17):

(1) —(CH₂)ₙ₁—C(═O)_OR^{cc1},

(2) —O—(CH₂)ₙ₂—C(═O)—OR^{cc2},

(3) —(CH₂)ₙ₃-ring P,

(4) —(CH₂)ₙ₄—C(═O)—NH—S(═O)₂—CH₃,

(5) C₁₋₆ alkyl group which may optionally be substituted with the same or different 1 to 5 substituents selected from Group C,

(6) C₃₋₆ cycloalkyl group which may optionally be substituted with the same or different 1 to 5 substituents selected from Group C,

(7) C₂₋₁₂ alkenyl group,

(8) C₂₋₁₂ alkynyl group,

(9) —NR^{cc3}R^{cc4},

(10) —OR^{cc5},

(11) —O—CH₂CH₂—OH,

(12) —O—CH₂C(═O)NH—CH₃,

R^{cc1} and R^{cc2} are each independently hydrogen atom or C₁₋₆ alkyl group;
R^{cc3} and R^{cc4} are each independently,
hydrogen atom,
C₁₋₆ alkyl group,
C₁₋₆ alkylcarbonyl group,
benzyloxycarbonyl group, or
C₁₋₆ alkyl group which is substituted with 1 to 3 hydroxyl groups;
R^{cc5} is
   hydrogen atom,
   C₁₋₆ alkyl group which may optionally be substituted with the same or different 1 to 5 substituents selected from Group C, or
   benzyl group;
ring P is monocyclic heterocycle which is a carboxylic acid equivalent wherein the carboxylic acid equivalent may optionally be substituted;
n1 is an integer selected from 0 or 1 to 3;
n2 is an integer selected from 0 or 1 to 3;
n3 is an integer selected from 1 to 3; and
n4 is an integer selected from 1 to 3;
   each R^{d} is the same or different and selected from the following (1) to (13):

(1) halogen atom,

(2) C₁₋₆ alkyl group which may optionally be substituted with the same or different 1 to 5 substituents selected from Group B,

(3) C₃₋₇ cycloalkyl group which may optionally be substituted with the same or different 1 to 5 substituents selected from Group B,

(4) C₂₋₆ alkenyl group,

(5) cyano group,

(6) —C(═O)—OR^{dd1},

(7) —C(═O)—NR^{dd2}R^{dd3},

(8) —OR^{dd4},

(9) —NR^{dd5}—C(═O)—R^{dd6},

(10) —NR^{dd7}—C(═O)—NR^{dd8}R^{dd9},

(11) —NR^{dd10}—S(═O)₂—R^{dd11},

(12) —NR^{dd12}—S(═O)₂—NR^{dd13}R^{dd14},

(13) —NR^{dd15}R^{dd16},
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₈ alkyl group which may optionally be substituted with the same or different 1 to 5 substituents selected from Group B;
   R^{e} is hydrogen atom or C₁₋₆ alkyl group;
   R^{f} is hydrogen atom or C₁₋₆ alkyl group;
   each R^{j} is the same or different C₁₋₁₂ alkyl group which may optionally be substituted with the same or different 1 to 5 substituents selected from Group A;
   Q is selected from the following (1) to (9):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,

(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;

Y is selected from the following (1) to (3):

(1) single bond,

(2) —S(═O)₂—,

(3) C₁₋₃ alkylene which may optionally be substituted with 1 to 3 hydroxyl groups;

m is each independently an integer selected from 0 or 1 to 5;
n^{j} is each independently 0, 1 or 2;
Group A consists of the following (a) to (m):

(a) alkyl group which may optionally be substituted with the same or different 1 to 5 substituents selected from Group AA,

(b) halogen atom,

(c) phenyl group,

(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,

(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(g) C₃₋₇ cycloalkyl group which may optionally be substituted with the same or different 1 to 5 substituents selected from Group AA,

(h) —C(═O)—NR^{A1}R^{A2},

(i) —C(═O)—OR^{A3},

(j) —C(═O)—R^{A4},

(k) —OR^{A5},

(l) —NR^{A6}R^{A7},

(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
   hydrogen atom,
   C₁₋₆ alkyl group which may optionally be substituted with the same or different 1 to 6 halogen atoms,
   C₃₋₇ cycloalkyl group which may optionally be substituted with the same or different 1 to 5 substituents selected from Group AA,
   benzyl group which may optionally be substituted with the same or different 1 to 5 substituents selected from Group AA,
   phenyl group which may optionally be substituted with the same or different 1 to 5 substituents selected from Group AA,
   C₁₋₆ alkylcarbonyl group,
   C₁₋₆ alkylsulfonyl group, or
   C₂₋₆ alkenyl group; and
R^{A8} is each independently C₁₋₆ alkyl group;
   Group AA consists of the following (a) to (o):

(a) halogen atom,

(b) C₁₋₃ alkyl group which may optionally be substituted with the same or different 1 to 6 halogen atoms,

(c) —OR^{AA1},

(d) —C(═O)—NR^{AA2}R^{AA3},

(e) —C(═O)—OR^{AA4},

(f) —O—C(═O)—R^{AA5},

(g) —C(═O)—R^{AA6},

(h) ═O,

(i) C₃₋₇ cycloalkyl group,

(j) phenyl group which may optionally be substituted with the same or different 1 to 5 C₁₋₃ alkyl groups,

(k) —NR^{AA7}R^{AA8},

(l) —NR^{AA9}—C(═O)—R^{AA10},

(m) —NR^{AA11}—C(═O)—NR^{AA12}R^{AA13},

(n) —NR^{AA14}—S(═O)₂—R^{AA15},

(o) —NR^{AA16}—S(═O)₂—NR^{AA17}R^{AA18};

R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):

(a) halogen atom,

(b) C₃₋₇ cycloalkyl group

(c) —OR^{B1},

(d) —C(═O)—NR^{B2}R^{B3},

(e) —C(═O)—OR^{B4},

(f) C₁₋₆ alkyl group,

(g) —NR^{B5}R^{B6},

(h) —NR^{B7}—C(═O)—R^{B8},

(i) —NR^{B9}—C(═O)—NR¹⁰R^{B11},

(j) —NR^{B12}—S(═O)₂—R^{B13},

(k) —NR^{B14}—S(═O)₂—NR^{B15}R^{B16};
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently hydrogen atom or C₁₋₆ alkyl group;
   Group C consists of the following (a) to (k):

(a) phenyl group which may optionally be substituted with the same or different 1 to 5 substituents selected from Group CC,

(b) cyano group,

(c) halogen atom,

(d) —OR^{CD1},

(e) —NR^{CD2}R^{CD3},

(f) —C(═O)—NR^{CD4}R^{CD5},

(i) saturated heteromonocyclic group which may optionally be substituted with the same or different 1 to 5 substituents selected from Group CC wherein the saturated heteromonocyclic ring comprises the same or different 1 to 4 hetero atoms selected from nitrogen atom, oxygen atom, or sulfur atom besides carbon atom, and is 4 to 6-membered, excluding ring P,

(j) unsaturated heteromonocyclic group which may optionally be substituted with the same or different 1 to 5 substituents selected from Group CC wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered, excluding ring P,

R^{CD1}, R^{CD2}, and R^{CD3} are each independently,
   hydrogen atom,
   C₁₋₆ alkyl group,
   C₁₋₆ alkylcarbonyl group,
   benzyl group,
   —C(═O)—O—CH₂-phenyl,
   —C(═O)—N(CH₃)₂,
   —C(═O)—C(OH) (CH₃)₂,
   —C(═O)—CH₂—O—CH₃, or
   —C(═O)—CH₂—CN; and
R^{CD4} and R^{CD5} are each independently hydrogen atom or C₁₋₆ alkyl group;
   Group CC consists of the following (a) to (c):

(a) halogen atom,

(b) C₁₋₃ alkyl group which may optionally be substituted with the same or different 1 to 6 halogen atoms,

(c) —OR^{AA1},
R^{AA1} is each independently hydrogen atom or C₁₋₆ alkyl group;
   provided that when R^{a} is

(1) phenyl group which may optionally be substituted with the same or different 1 to 5 substituents selected from Group A,

(2) C₉₋₁₀ fused carbocyclic group which may optionally be substituted with the same or different 1 to 5 substituents selected from Group A, or

(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

Q is selected from the following (1) to (6):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;

further provided that when R^{b} is hydrogen atom,
Q is selected from the following (1) to (5):

(1) phenyl group,

(2) C₃₋₇ cycloalkyl group,

(3) C₉₋₁₀ fused carbocyclic group,

(4) cross-linked C₅₋₁₂ cycloalkyl group,

(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;

and further provided that when

is

R^{c} is selected from the following (1) to (15):

(1) —(CH₂)ₙ₃-ring P,

(2) —(CH₂)ₙ₄—C(═O)—NH—S(═O)₂—CH₃,

(3) C₁₋₅ alkyl group which may optionally be substituted with the same or different 1 to 5 substituents selected from Group C,

(4) C₃₋₆ cycloalkyl group which may optionally be substituted with the same or different 1 to 5 substituents selected from Group C,

(5) C₂₋₁₂ alkenyl group.

(6) C₂₋₁₂ alkynyl group,

(7) —NR^{cc3}R^{cc4},

(8) —OR^{cc5},

(9) —O—CH₂CH₂—OH,

(10) —O—CH₂C(═O)NH—CH₃,

[107] A compound represented by the following formula:

or a pharmaceutically acceptable salt thereof, wherein

is unsaturated heteromonocyclic group selected from the following (i) to (ii):

R^{a} is selected from the following (1) to (12):

(1) C₁₋₁₂ alkyl group which may optionally be substituted with the same or different 1 to 5 substituents selected from Group A,

(2) C₃₋₇ cycloalkyl group which may optionally be substituted with the same or different 1 to 5 substituents selected from Group A,

(3) C₂₋₁₂ alkenyl group which may optionally be substituted with the same or different 1 to 5 substituents selected from Group A,

(4) C₂₋₁₂ alkynyl group which may optionally be substituted with the same or different 1 to 5 substituents selected from Group A,

(5) C₅₋₁₁ spirocyclic cycloalkyl group which may optionally be substituted with the same or different 1 to 5 substituents selected from Group A,

(6) cross-linked C₅₋₁₂ cycloalkyl group which may optionally be substituted with the same or different 1 to 5 substituents selected from Group A,

(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,

(8) phenyl group which may optionally be substituted with the same or different 1 to 5 substituents selected from Group A,

(9) C₉₋₁₀ fused carbocyclic group which may optionally be substituted with the same or different 1 to 5 substituents selected from Group A,

(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,

(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;

R^{b} is selected from the following (1) to (6):

(1) C₁₋₆ alkyl group which may optionally be substituted with the same or different 1 to 5 halogen atoms,

(2) C₃₋₇ cycloalkyl group which may optionally be substituted with the same or different 1 to 5 substituents selected from the group consisting of halogen atom and C₁₋₆ alkyl group,

(3) —CH═CH—C(═O)—OR^{bb1},

(4) —CH₂—CH₂—C(═O)—OR^{bb2},

(5) —CH₂—O—CH₂—C(═O)—OR^{bb3},

(6) hydrogen atom;
R^{bb1}, R^{bb2}, and R^{bb3} are each independently hydrogen atom or C₁₋₆ alkyl group;
   R^{c} is selected from the following (1) to (15):

(1) —(CH₂)ₙ₃-ring P,

(2) —(CH₂)ₙ₄—C(═O)—NH—S(═O)₂—CH₃,

(3) C₁₋₆ alkyl group which may optionally be substituted with the same or different 1 to 5 substituents selected from Group C,

(4) C₃₋₆ cycloalkyl group which may optionally be substituted with the same or different 1 to 5 substituents selected from Group C,

(5) C₂₋₁₂ alkenyl group,

(6) C₂₋₁₂ alkynyl group,

(7) —NR^{cc3}R^{cc4},

(8) —OR^{cc5},

(9) —O—CH₂CH₂—OH,

(10) —O—CH₂C(═O)NH—CH₃,

R^{cc3} and R^{cc4} are each independently,
hydrogen atom,
C₁₋₆ alkyl group,
C₁₋₆ alkylcarbonyl group,
benzyloxycarbonyl group, or
C₁₋₆ alkyl group which is substituted with 1 to 3 hydroxyl groups;
R^{cc5} is
   hydrogen atom,
   C₁₋₆ alkyl group which may optionally be substituted with the same or different 1 to 5 substituents selected from Group C, or
   benzyl group;
ring P is monocyclic heterocycle which is a carboxylic acid equivalent wherein the carboxylic acid equivalent may optionally be substituted;
n3 is an integer selected from 1 to 3; and
n4 is an integer selected from 1 to 3;
   each R^{d} is the same or different and selected from the following (1) to (13):

(1) halogen atom,

(2) C₁₋₆ alkyl group which may optionally be substituted with the same or different 1 to 5 substituents selected from Group B,

(3) C₃₋₇ cycloalkyl group which may optionally be substituted with the same or different 1 to 5 substituents selected from Group B,

(4) C₂₋₆ alkenyl group,

(5) cyano group,

(6) —C(═O)—OR^{dd1},

(7) —C(═O) —NR^{dd2}R^{dd3},

(8) —OR^{dd4},

(9) —NR^{dd5}—C(═O)—R^{dd6},

(10) —NR^{dd7}—C(═O)—NR^{dd8}R^{dd9},

(11) —NR^{dd10}—S(═O)₂—R^{dd11},

(12) —NR^{dd12}—S(═O)₂—NR^{dd13}R^{dd14},

(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
   R^{e} is hydrogen atom or C₁₋₆ alkyl group;
   Q is selected from the following (1) to (9):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,

(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;

Y is selected from the following (1) to (3):

(1) single bond,

(2) —S(═O)₂—,

(3) C₁₋₃ alkylene which may optionally be substituted with 1 to 3 hydroxyl groups;

m is each independently an integer selected from 0 or 1 to 5;
Group A consists of the following (a) to (m):

(a) C₁₋₇ alkyl group which may optionally be substituted with the same or different 1 to 5 substituents selected from Group AA,

(b) halogen atom,

(c) phenyl group,

(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,

(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(g) C₃₋₇ cycloalkyl group which may optionally be substituted with the same or different 1 to 5 substituents selected from Group AA,

(h) —C(═O)—NR^{A1}R^{A2},

(i) —C(═O)—OR^{A3},

(j) —C(═O)—R^{A4},

(k) —OR^{A5},

(l) —NR^{A6}R^{A7},

(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
   hydrogen atom,
   C₁₋₆ alkyl group which may optionally be substituted with the same or different 1 to 6 halogen atoms,
   C₃₋₇ cycloalkyl group which may optionally be substituted with the same or different 1 to 5 substituents selected from Group AA,
   benzyl group which may optionally be substituted with the same or different 1 to 5 substituents selected from Group AA,
   phenyl group which may optionally be substituted with the same or different 1 to 5 substituents selected from Group AA,
   C₁₋₆ alkylcarbonyl group,
   C₁₋₆ alkylsulfonyl group, or
   C₂₋₆ alkenyl group; and
R^{A8} is each independently C₁₋₆ alkyl group;
   Group AA consists of the following (a) to (o):

(a) halogen atom,

(b) C₁₋₃ alkyl group which may optionally be substituted with the same or different 1 to 6 halogen atoms,

(c) —OR^{AA1},

(d) —C(═O)—NR^{AA2}R^{AA3},

(e) —C(═O)—OR^{AA4},

(f) —O—C(═O)—R^{AA5},

(g) —C(═O)—R^{AA6},

(h) ═O,

(i) C₃₋₇ cycloalkyl group,

(j) phenyl group which may optionally be substituted with the same or different 1 to 5 C₁₋₃ alkyl groups,

(k) —NR^{AA7}R^{AA8},

(l) —NR^{AA9}—C(═O)—R^{AA10},

(m) —NR^{AA11}—C(═O)—NR^{AA12}R^{AA13},

(n) —NR^{AA14}—S(═O)₂—R^{AA15},

(o) —NR^{AA16}—S(═O)₂—NR^{AA17}R^{AA18};

R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):

(a) halogen atom,

(b) C₃₋₇ cycloalkyl group

(c) —OR^{B1},

(d) —C(═O)—NR^{B2}R^{B3},

(e) —C(═O)—OR^{B4},

(f) C₁₋₆ alkyl group,

(g) —NR^{B5}R^{B6},

(h) —NR^{B7}—C(═O)—R^{B8},

(i) —NR^{B9}—C(═O)—NR^{B10}R^{B11},

(j) —NR^{B12}—S(═O)₂—R^{B13},

(k) —NR^{B14}—S(═O)₂—NR^{B15}R^{B16};
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently hydrogen atom or C₁₋₆ alkyl group;
   Group C consists of the following (a) to (k):

(a) phenyl group which may optionally be substituted with the same or different 1 to 5 substituents selected from Group CC,

(b) cyano group,

(c) halogen atom,

(d) —OR^{CD1},

(e) —NR^{CD2}R^{CD3},

(f) —C(═O)—NR^{CD4}R^{CD5},

(i) saturated heteromonocyclic group which may optionally be substituted with the same or different 1 to 5 substituents selected from Group CC wherein the saturated heteromonocyclic ring comprises the same or different 1 to 4 hetero atoms selected from nitrogen atom, oxygen atom, or sulfur atom besides carbon atom, and is 4 to 6-membered, excluding ring P,

(j) unsaturated heteromonocyclic group which may optionally be substituted with the same or different 1 to 5 substituents selected from Group CC wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered, excluding ring P,

R^{CD1}, R^{CD2}, and R^{CD3} are each independently,
   hydrogen atom,
   C₁₋₆ alkyl group,
   C₁₋₆ alkylcarbonyl group,
   benzyl group,
   —C(═O)—O—CH₂-phenyl,
   —C(═O)—N(CH₃)₂,
   —C(═O)—C(OH) (CH₃)₂,
   —C(═O)—CH₂—O—CH₃, or
   —C(═O)—CH₂—CN; and
R^{CD4} and R^{CD5} are each independently hydrogen atom or C₁₋₆ alkyl group;
   Group CC consists of the following (a) to (c):

(a) halogen atom,

(b) C₁₋₃ alkyl group which may optionally be substituted with the same or different 1 to 6 halogen atoms,

(c) OR^{AA1},
R^{AA1} is each independently hydrogen atom or C₁₋₆ alkyl group;
   provided that when R^{a} is

(1) phenyl group which may optionally be substituted with the same or different 1 to 5 substituents selected from Group A,

(2) C₉₋₁₀ fused carbocyclic group which may optionally be substituted with the same or different 1 to 5 substituents selected from Group A, or

(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

Q is selected from the following (1) to (6):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;

and further provided that when R^{b} is hydrogen atom,
Q is selected from the following (1) to (5):

(1) phenyl group,

(2) C₃₋₇ cycloalkyl group,

(3) C₉₋₁₀ fused carbocyclic group,

(4) cross-linked C₅₋₁₂ cycloalkyl group,

(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered.

[108] A compound represented by the following formula:

or a pharmaceutically acceptable salt thereof, wherein

is unsaturated heteromonocyclic group selected from the following (i) to (ii):

each R^{au} is the same or different and selected from the following (1) or (2):

(1) C₇₋₁₂ alkyl group,

(2) C₁₋₁₂ alkyl group which is substituted with the same or different 1 to 5 substituents selected from Group AU;

R^{b} is selected from the following (1) to (6):

(1) C₁₋₆ alkyl group which may optionally be substituted with the same or different 1 to 5 halogen atoms,

(2) C₃₋₇ cycloalkyl group which may optionally be substituted with the same or different 1 to 5 substituents selected from the group consisting of halogen atom and C₁₋₆ alkyl group,

(3) —CH═CH—C(═O)—OR^{bb1},

(4) —CH₂—CH₂—C(═O)—OR^{bb2},

(5) —CH₂—O—CH₂—C(═O)—OR^{bb3},

(6) hydrogen atom;
R^{bb1}, R^{bb2}, and R^{bb3} are each independently hydrogen atom or C₁₋₆ alkyl group;
   R^{c} is selected from the following (1) or (2):

(1) —(CH₂)ₙ₁—C(═O)—OR^{cc1},

(2) —O—(CH₂)ₙ₂—C(═O)—OR^{cc2},

R^{cc1} and R^{cc2} are each independently hydrogen atom or C₁₋₆ alkyl group;
n1 is an integer selected from 0 or 1 to 3; and
n2 is an integer selected from 0 or 1 to 3;
   each R^{d} is the same or different and selected from the following (1) to (13):

(1) halogen atom,

(2) C₁₋₆ alkyl group which may optionally be substituted with the same or different 1 to 5 substituents selected from Group B,

(3) C₃₋₇ cycloalkyl group which may optionally be substituted with the same or different 1 to 5 substituents selected from Group B,

(4) C₂₋₆ alkenyl group,

(5) cyano group,

(6) —C(═O)—OR^{dd1},

(7) —C(═O)—N^{dd2}R^{dd3},

(8) —OR^{dd4},

(9) —NR^{dd5}—C(═O)—R^{dd6},

(10) —NR^{dd7}—C(═O)—NR^{dd8}R^{dd9},

(11) —NR^{dd10}—S(═O)₂—R^{dd11},

(12) —NR^{dd12}—S(═O)₂—NR^{dd13}R^{dd14},

(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
   R^{e} is hydrogen atom or C₁₋₆ alkyl group;
   Q is selected from the following (1) to (9):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,

(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;

cyclic moiety UU is selected from the following (1) or (2):

(1) C₃₋₇ cycloalkyl group,

(2) phenyl group;

Y is selected from the following (1) to (3):

(1) single bond,

(2) —S(═O)₂—,

(3) alkylene which may optionally be substituted with 1 to 3 hydroxyl groups;

m is each independently an integer selected from 0 or 1 to 5;
n^{u} is an integer selected from 0 or 1 to 3;
Group B consists of the following (a) to (k):

(a) halogen atom,

(b) C₃₋₇ cycloalkyl group

(c) —OR^{B1},

(d) —C(═O)—NR^{B2}R^{B3},

(e) —C(═O)—OR^{B4},

(f) C₁₋₆ alkyl group,

(g) —NR^{B5}R^{B6},

(h) —NR^{B7}—C(═O)—R^{B8},

(i) —NR^{B9}—C(═O)—NR^{B10}R^{B11},

(j) —NR^{B12}—S(═O)₂—R^{B13},

(k) —NR^{B14}—S(═O)₂—NR^{B15}R^{B16};
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently, hydrogen atom or C₁₋₆ alkyl group;
   Group AU consists of the following (a) to (j)

(a) —OR^{AU1},

(b) —C(═O)—OR^{AU2},

(c) —C(═O)—NR^{AU3}R^{AU4},

(d) phenyl group,

(e) C₃₋₇ cycloalkyl group,

(f) —NR^{AU5}R^{AU6},

(g) —NR^{AU7}—C(═O)—R^{AU8},

(h) —NR^{AU9}—C(═O)—NR^{AU10}R^{AU12},

(i) —NR^{AU13}—S(═O)₂—R^{AU14},

(j) —NR^{AU15}—S(═O)₂—NR^{AU16}R^{AU17},
R^{AU1}, R^{AU2}, R^{AU3}, R^{AU4}, R^{AU5}, R^{AU6}, R^{AU7}, R^{AU8}, R^{AU9}, R^{AU10}, R^{AU11}, R^{AU12}, R^{AU13}, R^{AU14}, R^{AU15}, R^{AU16} and R^{AU17} are each independently hydrogen atom or C₁₋₆ alkyl group;
   provided that when cyclic moiety UU is phenyl group,
   Q is selected from the following (1) to (6):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;

and further provided that when R^{b} is hydrogen atom,
Q is selected from the following (1) to (5):

(1) phenyl group,

(2) C₃₋₇ cycloalkyl group,

(3) C₉₋₁₀ fused carbocyclic group,

(4) cross-linked C₅₋₁₂ cycloalkyl group,

(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered.

[109] A compound represented by the following formula:

or a pharmaceutically acceptable salt thereof, wherein

is unsaturated heteromonocyclic group selected from the following (i) to (ii):

R^{a} is selected from the following (1) to (12):

(1) C₁₋₁₂ alkyl group which may optionally be substituted with the same or different 1 to 5 substituents selected from Group A,

(2) C₃₋₇ cycloalkyl group which may optionally be substituted with the same or different 1 to 5 substituents selected from Group A,

(3) C₂₋₁₂ alkenyl group which may optionally be substituted with the same or different 1 to 5 substituents selected from Group A,

(4) C₂₋₁₂ alkynyl group which may optionally be substituted with the same or different 1 to 5 substituents selected from Group A,

(5) C₅₋₁₁ spirocyclic cycloalkyl group which may optionally be substituted with the same or different 1 to 5 substituents selected from Group A,

(6) cross-linked C₅₋₁₂ cycloalkyl group which may optionally be substituted with the same or different 1 to 5 substituents selected from Group A,

(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,

(8) phenyl group which may optionally be substituted with the same or different 1 to 5 substituents selected from Group A,

(9) C₉₋₁₀ fused carbocyclic group which may optionally be substituted with the same or different 1 to 5 substituents selected from Group A,

(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,

(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;

R^{c} is selected from the following (1) or (2):

(1) —(CH₂)ₙ₁—C(═O)—OR^{cc1},

(2) —O—(CH₂)ₙ₂—C(═O)—OR^{cc2},

R^{cc1} and R^{cc2} are each independently hydrogen atom or C₁₋₆ alkyl group;
n1 is an integer selected from 0 or 1 to 3; and
n2 is an integer selected from 0 or 1 to 3;
   each R^{d} is the same or different and selected from the following (1) to (13):

(1) halogen atom,

(2) C₁₋₆ alkyl group which may optionally be substituted with the same or different 1 to 5 substituents selected from Group B,

(3) C₃₋₇ cycloalkyl group which may optionally be substituted with the same or different 1 to 5 substituents selected from Group B,

(4) C₂₋₆ alkenyl group,

(5) cyano group,

(6) —C(═O)—OR^{dd1},

(7) —C(═O)—NR^{dd2}R^{dd3},

(8) —OR^{dd4},

(9) —NR^{dd5}—C(═O)—R^{dd6},

(10) —NR^{dd7}—C(═O)—NR^{dd8}R^{dd9},

(11) —NR^{dd10}—S(═O)₂—R^{dd11},

(12) —NR^{dd12}—S(═O)₂—NR^{dd13}R^{dd14},

(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
   R^{e} is hydrogen atom or C₁₋₆ alkyl group;
   Q is selected from the following (1) to (9):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,

(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;

Y is selected from the following (1) to (3):

(1) single bond,

(2) —S(═O)₂—,

(3) C₁₋₃ alkylene which may optionally be substituted with 1 to 3 hydroxyl groups;

m is each independently an integer selected from 0 or 1 to 5;
Group A consists of the following (a) to (m):

(a) C₁₋₇ alkyl group which may optionally be substituted with the same or different 1 to 5 substituents selected from Group AA,

(b) halogen atom,

(c) phenyl group,

(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,

(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(g) C₃₋₇ cycloalkyl group which may optionally be substituted with the same or different 1 to 5 substituents selected from Group AA,

(h) —C(═O)—NR^{A1}R^{A2},

(i) —C(═O)—OR^{A3},

(j) —C(═O)—R^{A4},

(k) —OR^{A5},

(l) —NR^{A6}R^{A7},

(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
   hydrogen atom,
   C₁₋₆ alkyl group which may optionally be substituted with the same or different 1 to 6 halogen atoms,
   C₃₋₇ cycloalkyl group which may optionally be substituted with the same or different 1 to 5 substituents selected from Group AA,
   benzyl group which may optionally be substituted with the same or different 1 to 5 substituents selected from Group AA,
   phenyl group which may optionally be substituted with the same or different 1 to 5 substituents selected from Group AA,
   C₁₋₆ alkylcarbonyl group,
   C₁₋₆ alkylsulfonyl group, or
   C₂₋₆ alkenyl group; and
R^{A8} is each independently C₁₋₆ alkyl group;
   Group AA consists of the following (a) to (o):

(a) halogen atom,

(b) C₁₋₃ alkyl group which may optionally be substituted with the same or different 1 to 6 halogen atoms,

(c) —OR^{AA1},

(d) —C(═O)—NR^{AA2}R^{AA3},

(e) —C(═O)—OR^{AA4},

(f) —O—C(═O)—R^{AA5},

(g) —C(═O)—R^{AA6},

(h) ═O,

(i) C₃₋₇ cycloalkyl group,

(j) phenyl group which may optionally be substituted with the same or different 1 to 5 C₁₋₃ alkyl groups,

(k) —NR^{AA7}R^{AA8},

(l) —NR^{AA9}—C(═O)—R^{AA10},

(m) —NR^{AA11}—C(═O)—NR^{AA12}R^{AA13},

(n) —NR^{AA14}—S(═O)₂—R^{AA15},

(o) —NR^{AA16}—S(═O)₂—NR^{AA17}R^{AA18};

R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):

(a) halogen atom,

(c) —OR^{B1},

(d) —C(═O)—NR^{B2}R^{B3},

(e) —C(═O)—OR^{B4},

(f) C₁₋₆ alkyl group,

(g) —NR^{B5}R^{B6},

(h) —NR^{B7}—C(═O)—R^{B8},

(i) —NR^{B9}—C(═O)—NR^{B10}R^{B11},

(j) —NR^{B12}—S(═O)₂—R^{B13},

(k) —NR^{B14}—S(═O)₂—NR^{B15}R^{B16};

R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently, hydrogen atom or C₁₋₆ alkyl group;
provided that when R^{a} is

(1) phenyl group which may optionally be substituted with the same or different 1 to 5 substituents selected from Group A,

(2) C₉₋₁₀ fused carbocyclic group which may optionally be substituted with the same or different 1 to 5 substituents selected from Group A, or

(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

Q is selected from the following (1) to (6):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered.

[110] A compound represented by the following formula:

or a pharmaceutically acceptable salt thereof, wherein

is unsaturated heteromonocyclic group selected from the following (i) to

R^{a} is selected from the following (1) to (12):

(1) C₁₋₁₂ alkyl group which may optionally be substituted with the same or different 1 to 5 substituents selected from Group A,

(2) C₃₋₇ cycloalkyl group which may optionally be substituted with the same or different 1 to 5 substituents selected from Group A,

(3) C₂₋₁₂ alkenyl group which may optionally be substituted with the same or different 1 to 5 substituents selected from Group A,

(4) C₂₋₁₂ alkynyl group which may optionally be substituted with the same or different 1 to 5 substituents selected from Group A,

(5) C₅₋₁₁ spirocyclic cycloalkyl group which may optionally be substituted with the same or different 1 to 5 substituents selected from Group A,

(6) cross-linked C₅₋₁₂ cycloalkyl group which may optionally be substituted with the same or different 1 to 5 substituents selected from Group A,

(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,

(8) phenyl group which may optionally be substituted with the same or different 1 to 5 substituents selected from Group A,

(9) C₉₋₁₀ fused carbocyclic group which may optionally be substituted with the same or different 1 to 5 substituents selected from Group A,

(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,

(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;

R^{b} is selected from the following (1) to (6):

(1) C₂₋₆ alkyl group which may optionally be substituted with the same or different 1 to 5 halogen atoms,

(2) C₃₋₇ cycloalkyl group which may optionally substituted with the same or different 1 to 5 substituents selected from the group consisting of halogen atom and C₁₋₆ alkyl group,

(3) —CH═CH—C(═O)—OR^{bb1},

(4) —CH₂—CH₂—C(═O)—OR^{bb2},

(5) —CH₂—O—CH₂—C(═O)—OR^{bb3},

(6) hydrogen atom;
R^{bb1}, R^{bb2} and R^{bb3} are each independently hydrogen atom or C₁₋₆ alkyl group;
   R^{b} is selected from the following (1) or (2):

(1) —(CH₂)ₙ₁—C(═O)_OR^{cc1},

(2) —O—(CH₂)ₙ₂—C(═O)—OR^{cc2},

R^{cc2} and R^{cc2} are each independently hydrogen atom or C₁₋₆ alkyl group;
n1 is an integer selected from 0 or 1 to 3; and
n2 is an integer selected from 0 or 1 to 3;
   each R^{d} is the same or different and selected from the following (1) to (13):

(1) halogen atom,

(2) C₁₋₆ alkyl group which may optionally be substituted with the same or different 1 to 5 substituents selected from Group B,

(3) C₃₋₇ cycloalkyl group which may optionally be substituted with the same or different 1 to 5 substituents selected from Group B,

(4) C₂₋₆ alkenyl group,

(5) cyano group,

(6) —C(═O)—OR^{dd1},

(7) —C(═O)—NR^{dd2}R^{dd3},

(8) —OR^{dd4},

(9) —NR^{dd5}—C(═O)—R^{dd6},

(10) —NR^{dd7}—C(═O)—NR^{dd8}R^{dd9},

(11) —NR^{dd10}—S(═O)₂—R^{dd11},

(12) —NR^{dd12}—S(═O)₂—NR^{dd13}R^{dd14},

(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
   R^{e} is hydrogen atom or C₁₋₆ alkyl group;
   Q is selected from the following (1) to (9):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,

(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;

m is each independently an integer selected from 0 or 1 to 5;
Group A consists of the following (a) to (m):

(a) C₁₋₇ alkyl group which may optionally be substituted with the same or different 1 to 5 substituents selected from Group AA,

(b) halogen atom,

(c) phenyl group,

(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,

(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(g) C₃₋₇ cycloalkyl group which may optionally be substituted with the same or different 1 to 5 substituents selected from Group AA,

(h) —C(═O)—NR^{A1}R^{A2},

(i) —C(═O)—OR^{A3},

(j) —C(═O)—R^{A4},

(k) —OR^{A5},

(l) —NR^{A6}R^{A7},

(m) —S(═O)₂—,
R^{A1}, R^{A2}, R^{A3} and R^{A4} are each independently hydrogen atom or C₁₋₆ alkyl group;
R^{A5}, R^{A6}, and R^{A7} are each independently:
   hydrogen atom,
   C₁₋₆ alkyl group which may optionally be substituted with the same or different 1 to 6 halogen atoms,
   C₃₋₇ cycloalkyl group which may optionally be substituted with the same or different 1 to 5 substituents selected from Group AA,
   benzyl group which may optionally be substituted with the same or different 1 to 5 substituents selected from Group AA,
   phenyl group which may optionally be substituted with the same or different 1 to 5 substituents selected from Group AA,
   C₁₋₆ alkylcarbonyl group.
   C₁₋₆ alkylsulfonyl group, or
   C₂₋₆ alkenyl group; and
R^{A8} is each independently C₁₋₆ alkyl group;
   Group AA consists of the following (a) to (o):

(a) halogen atom,

(b) C₁₋₃ alkyl group which may optionally be substituted with the same or different 1 to 6 halogen atoms,

(c) —OR^{AA1},

(d) —C(═O)—NR^{AA2}R^{AA3},

(e) —C(═O)—OR^{AA4},

(f) —O—C(═O)—R^{AA5},

(g) —C(═O)—R^{AA6},

(h) ═O,

(i) C₃₋₇ cycloalkyl group,

(j) phenyl group which may optionally be substituted with the same or different 1 to 5 C₁₋₃ alkyl groups,

(k) —NR^{AA7}R^{AA8},

(l) —NR^{AA9}—C(═O)—R^{AA10},

(m) —NR^{AA11}—C(═O)—NR^{AA12}R^{AA13},

(n) —NR^{AA14}—S(═O)₂—R^{AA15},

(o) —NR^{AA16}—S(═O)₂—NR^{AA17}R^{AA18};

R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):

(c) —OR^{B1},

(d) —C(═O)—NR^{B2}R^{B3},

(e) —C(═O)—OR^{B4},

(f) C₁₋₆ alkyl group,

(g) —NR^{B5}R^{B6},

(h) —NR^{B7}—C(═O)—R^{B8},

(i) —NR^{B9}—C(═O)—NR^{B10}R^{B11},

(j) —NR^{B12}—S(═O)₂—R^{B13},

(k) —NR^{B14}—S(═O)₂—NR^{B15}R^{B16};

R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently, hydrogen atom or C₁₋₆ alkyl group;
provided that when R^{a} is

(1) phenyl group which may optionally be substituted with the same or different 1 to 5 substituents selected from Group A,

(2) C₉₋₁₀ fused carbocyclic group which may optionally be substituted with the same or different 1 to 5 substituents selected from Group A, or

(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

Q is selected from the following (1) to (6):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;

and further provided that when R^{b} is hydrogen atom,
Q is selected from the following (1) to (5):

(1) phenyl group,

(2) C₃₋₇ cycloalkyl group,

(3) C₉₋₁₀ fused carbocyclic group,

(4) cross-linked C₅₋₁₂ cycloalkyl group,

(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered.

[111] A compound represented by the following formula:

or a pharmaceutically acceptable salt thereof, wherein

is unsaturated heteromonocyclic group selected from the following (i) to (ii):

R^{a} is selected from the following (1) to (12):

(1) C₁₋₁₂ alkyl group which may optionally be substituted with the same or different 1 to 5 substituents selected from Group A,

(2) C₃₋₇ cycloalkyl group which may optionally be substituted with the same or different 1 to 5 substituents selected from Group A,

(3) C₂₋₁₂ alkenyl group which may optionally be substituted with the same or different 1 to 5 substituents selected from Group A,

(4) C₂₋₁₂ alkynyl group which may optionally be substituted with the same or different 1 to 5 substituents selected from Group A,

(5) C₅₋₁₁ spirocyclic cycloalkyl group which may optionally be substituted with the same or different 1 to 5 substituents selected from Group A,

(6) cross-linked C₃₋₁₂ cycloalkyl group which may optionally be substituted with the same or different 1 to 5 substituents selected from Group A,

(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,

(8) phenyl group which may optionally be substituted with the same or different 1 to 5 substituents selected from Group A,

(9) C₉₋₁₀ fused carbocyclic group which may optionally be substituted with the same or different 1 to 5 substituents selected from Group A,

(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,

(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;

R^{b} is selected from the following (1) to (6):

(1) C₁₋₆ alkyl group which may optionally be substituted with the same or different 1 to 5 halogen atoms,

(2) C₃₋₇ cycloalkyl group which may optionally be substituted with the same or different 1 to 5 substituents selected from the group consisting of halogen atom and C₁₋₆ alkyl group,

(3) —CH═CH—C(═O)—OR^{bb1},

(4) —CH₂—CH₂—C(═O)—OR^{bb2},

(5) —CH₂—O—CH₂—C(═O)—OR^{bb3},

(6) hydrogen atom;
R^{bb1}, R^{bb2} and R^{bb3} are each independently hydrogen atom or C₁₋₆ alkyl group;
   R^{c} is selected from the following (1) or (2):

(1) —(CH₂)ₙ₁—C(═O)—OR^{cc1},

(2) —O—(CH₂)ₙ₂—C(═O)—OR^{cc2},

R^{cc1} and R^{cc2} are each independently hydrogen atom or C₁₋₆ alkyl group;
n1 is an integer selected from 0 or 1 to 3; and
n2 is an integer selected from 0 or 1 to 3;
   each R^{d} is the same or different and selected from the following (1) to (13):

(1) halogen atom,

(2) C₁₋₆ alkyl group which may optionally be substituted with the same or different 1 to 5 substituents selected from Group B,

(3) C₃₋₇ cycloalkyl group which may optionally be substituted with the same or different 1 to 5 substituents selected from Group B,

(4) C₂₋₆ alkenyl group,

(5) cyano group,

(6) —C(═O)—OR^{dd1},

(7) —C(═O)—NR^{dd2}R^{dd3},

(8) —OR^{dd4},

(9) —NR^{dd5}—C(═O)—R^{dd6},

(10) —NR^{dd7}—C(═O)—NR^{dd8}R^{dd9},

(11) —NR^{dd10}—S(═O)₂—R^{dd11},

(12) —NR^{dd12}—S(═O)₂—NR^{dd13}R^{dd14},

(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₂₋₆ alkyl group which may optionally be substituted with the same or different 1 to 5 substituents selected from Group B;
   R^{e} is hydrogen atom or C₁₋₆ alkyl group;
   Q is selected from the following (1) to (7):

(1) C₃₋₇ cycloalkyl group,

(2) C₉₋₁₀ fused carbocyclic group selected from the group consisting of indanyl group and 1,2,3,4-tetrahydronaphthyl group,

(3) cross-linked C₅₋₁₂ cycloalkyl group,

(4) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,

(5) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(6) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(7) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;

Y is selected from the following (1) to (3):

(1) single bond,

(2) —S(═O)₂—,

(3) C₁₋₃ alkylene which may optionally be substituted with 1 to 3 hydroxyl groups;

m is each independently an integer selected from 0 or 1 to 5;
Group A consists of the following (a) to (m):

(a) alkyl group which may optionally be substituted with the same or different 1 to 5 substituents selected from Group AA,

(b) halogen atom,

(c) phenyl group,

(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,

(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(g) C₃₋₇ cycloalkyl group which may optionally be substituted with the same or different 1 to 5 substituents selected from Group AA,

(h) —C(═O)—NR^{A1}R^{A2},

(i) —C(═O)—OR^{A3},

(j) —C(═O)—R^{A4},

(k) —OR^{A5},

(l) —NR^{A6}R^{A7},

(m) —S(═O)₂—,
R^{A1}, R^{A2}, R^{A3} and R^{A4} are each independently hydrogen atom or C₁₋₆ alkyl group;
R^{A5}, R^{A6}, and R^{A7} are each independently:
   hydrogen atom,
   C₁₋₆ alkyl group which may optionally be substituted with the same or different 1 to 6 halogen atoms,
   C₃₋₇ cycloalkyl group which may optionally be substituted with the same or different 1 to 5 substituents selected from Group AA,
   benzyl group which may optionally be substituted with the same or different 1 to 5 substituents selected from Group AA,
   phenyl group which may optionally be substituted with the same or different 1 to 5 substituents selected from Group AA,
   C₁₋₆ alkylcarbonyl group,
   C₁₋₆ alkylsulfonyl group, or
   C₂₋₆ alkenyl group; and
R^{A8} is each independently C₁₋₆ alkyl group;
   Group AA consists of the following (a) to (o):

(a) halogen atom,

(b) C₁₋₃ alkyl group which may optionally be substituted with the same or different 1 to 6 halogen atoms,

(c) —OR^{AA1},

(d) —C(═O)—NR^{AA2}R^{AA3},

(e) —C(═O)—OR^{AA4},

(f) —O—C(═O)—R^{AA5},

(g) —C(═O)—R^{AA6},

(h) ═O,

(i) C₃₋₇ cycloalkyl group,

(j) phenyl group which may optionally be substituted with the same or different 1 to 5 C₁₋₃ alkyl groups,

(k) —NR^{AA7}R^{AA8},

(l) —NR^{AA9}—C(═O)—R^{AA10},

(m) —NR^{AA11}—C(═O)—NR^{AA12}R^{AA13},

(n) —NR^{AA14}—S(═O)₂—R^{AA15},

(o) —NR^{AA16}—S(═O)₂—NR^{AA17}R^{AA18};

R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):

(a) halogen atom,

(b) C₃₋₇ cycloalkyl group

(c) —OR^{B1},

(d) —C(═O)—NR^{B2}R^{B3},

(e) —C(═O)—OR^{B4},

(f) C₁₋₆ alkyl group,

(g) —NR^{B5}R^{B6},

(h) —NR^{B7}—C(═O)—R^{B8},

(i) —NR^{B9}—C(═O)—NR^{B10}R^{B11},

(j) —NR^{B12}—S(═O)₂—R^{B13},

(k) —NR^{B14}—S(═O)₂—NR^{B15}R^{B16};

R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently, hydrogen atom or C₁₋₆ alkyl group;
provided that when R^{a} is

(1) phenyl group which may optionally be substituted with the same or different 1 to 5 substituents selected from Group A,

(2) C₉₋₁₀ fused carbocyclic group which may optionally be substituted with the same or different 1 to 5 substituents selected from Group A, or

(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

Q is selected from the following (1) to (4):

(1) C₃₋₇ cycloalkyl group,

(2) C₉₋₁₀ fused carbocyclic group selected from the group consisting of indanyl group and 1,2,3,4-tetrahydronaphthyl group,

(3) cross-linked C₅₋₁₂ cycloalkyl group,

(4) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;

and further provided that when R^{b} is hydrogen atom,
Q is selected from the following (1) to (4):

(1) C₃₋₇ cycloalkyl group,

(2) C₉₋₁₀ fused carbocyclic group selected from the group consisting of indanyl group and 1,2,3,4-tetrahydronaphthyl group,

(3) cross-linked C₅₋₁₂ cycloalkyl group,

(4) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered.

[112] The compound according to any one of [101] to and [109] to [111], or a pharmaceutically acceptable salt thereof, wherein
R^{a} is selected from the following (1) to (4)

(1) C₁₋₁₂ alkyl group which may optionally be substituted with the same or different 1 to 5 substituents selected from Group A,

(2) C₃₋₇ cycloalkyl group which may optionally be substituted with the same or different 1 to 5 substituents selected from Group A,

(3) phenyl group which may optionally be substituted with the same or different 1 to 5 substituents selected from Group A,

(4) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered.

[113] The compound according to any one of [101] to [107] and [109] to [111], or a pharmaceutically acceptable salt thereof, wherein
R^{a} is selected from the following (1) to (3)

(1) C₁₋₁₂ alkyl group,

(2) cyclobutyl group substituted with C₁₋₇ alkyl group,

(3) phenyl group substituted with C₁₋₇ alkyl group.

[114] The compound according to any one of [101] to [108] and [110] to [111], or a pharmaceutically acceptable salt thereof, wherein
R^{b} is selected from the following (1) or (2)

(1) C₁₋₆ alkyl group which may optionally be substituted with the same or different 1 to 5 halogen atoms,

(2) C₃₋₇ cycloalkyl group which may optionally be substituted with the same or different 1 to 5 substituents selected from the group consisting of halogen atom and C₁₋₆ alkyl group.

[115] The compound according to any one of [101] to [108] and [110] to [111], or a pharmaceutically acceptable salt thereof, wherein
R^{b} is selected from the following (1) or (2)

(1) C₁₋₃ alkyl group which may optionally be substituted with the same or different 1 to 5 halogen atoms,

(2) cyclopropyl group.

[116] The compound according to any one of [101] to [109] and [111], or a pharmaceutically acceptable salt thereof, wherein R^{e} is hydrogen atom, and Y is single bond.
[117] The compound according to any one of [101] to [111], or a pharmaceutically acceptable salt thereof, wherein Q is phenyl group or C₉₋₁₀ fused carbocyclic group.
[118] A pharmaceutical composition comprising the compound according to any one of [101] to [117] or a pharmaceutically acceptable salt thereof, and pharmaceutically acceptable carrier.
[119] A RORγ antagonist comprising the compound according to any one of [101] to [117] or a pharmaceutically acceptable salt thereof.
[120] A medicament for treating or preventing a disease selected from the group consisting of autoimmune disease, allergic disease, dry eye, fibrosis, and metabolic disease, comprising the compound according to any one of [101] to [117] or a pharmaceutically acceptable salt thereof.
[121] The medicament according to [120] wherein the autoimmune disease is selected from the group consisting of rheumatoid arthritis, psoriasis, inflammatory bowel disease, multiple sclerosis, systemic lupus erythematosus, ankylosing spondylitis, uveitis, polymyalgia rheumatica, and type I diabetes.
[122] The medicament according to [120] wherein the metabolic disease is diabetes.
[123] A method of inhibiting RORγ in a mammal, comprising administering to said mammal a therapeutically effective amount of the compound according to any one of [101] to [117] or a pharmaceutically acceptable salt thereof.
[124] A method of treating or preventing a disease in a mammal selected from the group consisting of autoimmune disease, allergic disease, dry eye, fibrosis, and metabolic disease, comprising administering to said mammal a therapeutically effective amount of the compound according to any one of [101] to [117] or a pharmaceutically acceptable salt thereof.
[125] The method according to [124] wherein the autoimmune disease is selected from the group consisting of rheumatoid arthritis, psoriasis, inflammatory bowel disease, multiple sclerosis, systemic lupus erythematosus, ankylosing spondylitis, uveitis, polymyalgia rheumatica, and type I diabetes.
[126] The method according to [124] wherein the metabolic disease is diabetes.
[127] A pharmaceutical composition for treating or preventing a disease selected from the group consisting of autoimmune disease, allergic disease, dry eye, fibrosis, and metabolic disease, which comprises:

(a) the compound according to any one of [101] to [117] or a pharmaceutically acceptable salt thereof, and

(b) at least one additional medicament for treating or preventing a disease selected from the group consisting of autoimmune disease, allergic disease, dry eye, fibrosis, and metabolic disease.

[128] A combination drug comprising:

(a) the compound according to any one of [101] to [117] or a pharmaceutically acceptable salt thereof, and

(b) at least one additional medicament for treating or preventing a disease selected from the group consisting of autoimmune disease, allergic disease, dry eye, fibrosis, and metabolic disease,

wherein the compound of (a) and the additional medicament of (b) may be administered simultaneously, separately or consecutively.
[129] Use of the compound according to any one of [101] to [117] or a pharmaceutically acceptable salt thereof in the manufacture of a RORγ antagonist.
[130] Use of the compound according to any one of [101] to [117] or a pharmaceutically acceptable salt thereof in the manufacture of a medicament for treating or preventing a disease selected from the group consisting of autoimmune disease, allergic disease, dry eye, fibrosis, and metabolic disease.
[131] The use according to [130] wherein the autoimmune disease is selected from the group consisting of rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica, and type I diabetes.
[132] The use according to [130] wherein the metabolic disease is diabetes.
[133] The compound according to any one of [101] to [117] or a pharmaceutically acceptable salt thereof for use in treating or preventing a disease selected from the group consisting of autoimmune disease, allergic disease, dry eye, fibrosis, and metabolic disease.
[134] A commercial package comprising the medicament according to [120], and instructions which explain that the medicament can be used to treat and/or prevent a disease selected from the group consisting of autoimmune disease, allergic disease, dry eye, fibrosis, and metabolic disease.
[135] A commercial package comprising the combination drug according to [128], and instructions which explain that the combination drug can be used to treat and/or prevent a disease selected from the group consisting of autoimmune disease, allergic disease, dry eye, fibrosis, and metabolic disease.
[136] A medicament for treating or preventing a disease selected from the group consisting of autoimmune disease such as rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica and type I diabetes; allergic disease such as asthma; dry eye; fibrosis such as pulmonary fibrosis and primary biliary cirrhosis; and metabolic disease such as diabetes, comprising the compound according to any one of [101] to [117] or a pharmaceutically acceptable salt thereof.
[137] A pharmaceutical composition for treating or preventing a disease selected from the group consisting of autoimmune disease such as rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica and type I diabetes; allergic disease such as asthma; dry eye; fibrosis such as pulmonary fibrosis and primary biliary cirrhosis; and metabolic disease such as diabetes, comprising:

(a) the compound according to any one of [101] to [117] or a pharmaceutically acceptable salt thereof, and

(b) at least one additional medicament for treating or preventing a disease selected from the group consisting of autoimmune disease such as rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica and type I diabetes; allergic disease such as asthma; dry eye; fibrosis such as pulmonary fibrosis and primary biliary cirrhosis; and metabolic disease such as diabetes.

[138] A combination drug comprising:

(a) the compound according to any one of [101] to [117] or a pharmaceutically acceptable salt thereof, and

(b) at least one an additional medicament for treating or preventing a disease selected from the group consisting of autoimmune disease such as rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica and type I diabetes; allergic disease such as asthma; dry eye; fibrosis such as pulmonary fibrosis and primary biliary cirrhosis; and metabolic disease such as diabetes,

wherein the compound of (a) and the additional medicament of (b) may be administered simultaneously, separately or consecutively.
[139] A compound represented by the following formulas or a salt thereof:

wherein each symbol is as defined in [101].
[140] A compound represented by the following formulas or a salt thereof:

wherein each symbol is as defined in [102].
[141] A compound represented by the following formulas or a salt thereof:

wherein each symbol is as defined in [103].
[142] A compound represented by the following formulas or a salt thereof:

wherein each symbol is as defined in [104].
[143] The compound according to any one of [139] to [142] or a salt thereof, wherein
R^{a} is selected from the following (1) to (3)

(1) C₁₋₁₂ alkyl group,

(2) cyclobutyl group substituted with C₁₋₇ alkyl group,

(3) phenyl group substituted with C₁₋₇ alkyl group.

[144] The compound according to any one of [139] to [142] or a salt thereof, wherein
R^{b} is selected from the following (1) or (2)

(1) C₁₋₃ alkyl group which may optionally be substituted with the same or different 1 to 5 halogen atoms,

(2) cyclopropyl group.

[145] The compound according to any one of [139] to [144] or a salt thereof for the manufacture of a RORγ antagonist.
[146] The compound according to any one of [139] to [144] or a salt thereof in the manufacture of a medicament for treating or preventing a disease selected from the group consisting of autoimmune disease such as rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica and type I diabetes; allergic disease such as asthma; dry eye; fibrosis such as pulmonary fibrosis and primary biliary cirrhosis; and metabolic disease such as diabetes.

### DESCRIPTION OF EMBODIMENTS

The followings are definitions of terms that may be used in the specification.

The phrases “may be substituted” and “may optionally be substituted” mean to be substituted with the given number of given substituent(s) at any replaceable position(s) or not to be substituted (unsubstituted). The phrase “not substituted” herein means that all replaceable positions are occupied with hydrogen atoms.

For example, the phrase “C₁₋₆ alkyl group which may optionally be substituted with the same or different 1 to 5 substituents selected from Group A” includes both cases where C₁₋₆ alkyl group may be substituted with the same or different 1 to 5 substituents selected from Group A at any replaceable position(s) thereof and where C₁₋₆ alkyl group is not substituted (unsubstituted).

When a bond between a substituent and a ring is shown as a line crossing a bond connecting two atoms in the ring, such substituent may bind to any atom in the ring. When a substituent is shown without indicating the atom via which the substituent bind to the rest of the compound of a given formula, the substituent may be bonded via any atom in the substituent.

For example, the following aspects are included:

The term “halogen atom” includes for example, fluorine atom, chlorine atom, bromine atom, or iodine atom and the like.

The term “alkyl group” refers to a straight- or branched-chain saturated hydrocarbon group, and includes for example, C₁₋₁₂ alkyl group, C₁₋₈ alkyl group, C₁₋₆ alkyl group, C₁₋₄ alkyl group, C₁₋₃ alkyl group, C₅₋₁₂ alkyl group, C₅₋₈ alkyl group which have 1 to 12, 1 to 8, 1 to 6, 1 to 4, 1 to 3, 5 to 12, and 5 to 8 carbon atoms, respectively. Preferred examples of alkyl group include “C₁₋₃ alkyl group”, “C₁₋₆ alkyl group”. Examples of “C₁₋₃ alkyl group” include methyl group, ethyl group, propyl group, and isopropyl group. Examples of “C₁₋₆ alkyl group” include butyl group, isobutyl group, sec-butyl group, tert-butyl group, pentyl group, isopentyl group, neopenty group, tert-pentyl group, 1-ethylpropyl group, hexyl group, isohexyl group, 1,1-dimethylbutyl group, 2,2-dimethylbutyl group, 3,3-dimethylbutyl group, 2-ethylbutyl group, and 1,1-dimethyl-2-methylpropyl group, besides the above-mentioned examples of C₁₋₃ alkyl group. Examples of “C₁₋₁₂ alkyl group” include heptyl, octyl, nonyl, decyl, undecyl, and dodecyl, besides the above-mentioned examples, which may be a straight- or branched-chain.

The term “C₁₋₆ alkyl group” refers to a straight- or branched-chain saturated hydrocarbon group having 1 to 6 carbon atoms. Examples of “C₁₋₆ alkyl group” include methyl group, ethyl group, propyl group, isopropyl group, butyl group, isobutyl group, sec-butyl group, tert-butyl group, pentyl group, isopentyl group, neopenty group, tert-pentyl group, 1-ethylpropyl group, hexyl group, isohexyl group, 1,1-dimethylbutyl group, 2,2-dimethylbutyl group, 3,3-dimethylbutyl group, 2-ethylbutyl group, and 1,1-dimethyl-2-methylpropyl.

The term “C₃₋₇ cycloalkyl group” refers to a monocyclic saturated hydrocarbon group having 3 to 7 carbon atoms. Examples of “C₃₋₇ cycloalkyl group” include cyclopropyl group, cyclobutyl group, cyclopentyl group, cyclohexyl group, and cycloheptyl group.

The term “monocyclic heteroaromatic group” refers to a monocyclic heteroaromatic group which contains the same or different 1 to 4 hetero atoms selected from nitrogen atom, oxygen atom or sulfur atom besides carbon atom and is preferably 5- to 7-membered. The monocyclic heteroaromatic group may be attached via any available nitrogen or carbon atom in the ring. Examples of “monocyclic heteroaromatic group” include a monocyclic heteroaromatic group which contains the same or different 1 to 3 hetero atoms selected from nitrogen atom, oxygen atom or sulfur atom besides carbon atom and is 5- to 6-membered.

Examples of “monocyclic heteroaromatic group” include furyl group, thienyl group, pyrrolyl group, oxazolyl group, isoxazolyl group, thiazolyl group, isothiazolyl group, imidazolyl group, pyrazolyl group, oxadiazolyl group (1,2,5-oxadiazolyl group, 1,3,4-oxadiazolyl group, 1,2,4-oxadiazolyl group), thiadiazolyl group (1,2,5-thiadiazolyl group, 1,3,4-thiadiazolyl group, 1,2,4-thiadiazolyl group), triazolyl group (1,2,3-triazolyl group, 1,2,4-triazolyl group), tetrazolyl group, pyridyl group, pyrimidinyl group, pyridazinyl group, pyrazinyl group, and triazinyl.

Preferred examples of “monocyclic heteroaromatic group” include:

The more preferred examples of “monocyclic heteroaromatic group” include:

When the monocyclic heteroaromatic group is substituted, the monocyclic heteroaromatic group may be substituted at the carbon atom, and further the monocyclic heteroaromatic group may be substituted at nitrogen atom if the monocyclic heteroaromatic group has nitrogen atom(s) as a ring member. When nitrogen atom is contained in the monocyclic heteroaromatic ring as a ring member, the nitrogen atom may be quaternized with a substituent or may be oxidized to form a N-oxide derivative thereof.

The term “C₁₋₃ alkylene” refers to a bivalent group derived from a straight- or branched-chain C₁₋₃ alkyl, and includes for example, methylene, ethylene, trimethylene, and methylmethylene.

The term “C₁₋₆ alkylcarbonyl group” refers to a carbonyl with a C₁₋₆ alkyl. Examples of “C₁₋₆ alkylcarbonyl group” include acetyl group, propionyl group, butyryl group, isobutyryl group. The term “C₁₋₆ alkylsulfonyl group” refers to sulfonyl with C₁₋₆ alkyl. Examples of “C₁₋₆ alkylsulfonyl group” include methylsulfonyl group, ethylsulfonyl group, propylsulfonyl group, isopropylsulfonyl group (i.e. propane-2-sulfonyl group), n-butylsulfonyl group, isobutylsulfonyl group (i.e. 2-methylpropane-1-sulfonyl group).

The term “alkenyl group” refers to a straight- or branched-chain unsaturated hydrocarbon group having one or more double bonds. Examples of “alkenyl group” include vinyl group, 1-propenyl group, isopropenyl group, allyl group, methylpropenyl group (such as 1-methyl-1-propenyl group and 2-methyl-1-propenyl group), 1-butenyl group, 2-butenyl group, 3-butenyl group, methylbutenyl group (such as 1-methyl-1-butenyl group, 2-methyl-1-butenyl group, and 3-methyl-1-butenyl group), pentenyl group, methylpentenyl group, hexenyl group.

The term “C₂₋₁₂ alkenyl group” refers to alkenyl group having 2 to 12 carbon atoms, and the term “C₂₋₆ alkenyl group” refers to alkenyl group having 2 to 6 carbon atoms.

The term “C₂₋₁₂ alkynyl group” refers to a straight- or branched-chain unsaturated hydrocarbon group having 2 to 12 carbon atoms and one or more triple bonds. Examples of “C₂₋₁₂ alkynyl group” include ethynyl group, propynyl group (1-propynyl group, 2-propynyl group), butynyl group, pentynyl group, and hexynyl group. Preferred examples of “C₂₋₁₂ alkynyl group” include ethynyl group, and 1-propynyl group.

The term “C₅₋₁₁ spirocyclic cycloalkyl group” refers to spirocyclic cycloalkyl group having 5 to 11 carbon atoms. Examples of “C₅₋₁₁ spirocyclic cycloalkyl group” include:

The term “cross-linked C₅₋₁₂ cycloalkyl group” refers to cross-linked cycloalkyl group having 5 to 12 carbon atoms. Examples of “cross-linked C₅₋₁₂ cycloalkyl group” include:

Examples of “C₉₋₁₀ fused carbocyclic group” include:

Examples of “C₉₋₁₀ fused carbocyclic group selected from the group consisting of indanyl group and 1,2,3,4-tetrahydronaphthyl group” include:

The term “heteromonocyclic group” is “saturated heteromonocyclic group” or “unsaturated heteromonocyclic group”.

The term “saturated heteromonocyclic group” refers to monocyclic saturated heterocyclic group which contains the same or different 1 to 4 hetero atoms selected from nitrogen atom, oxygen atom or sulfur atom besides carbon atom and is 4- to 6-membered.

Examples of “saturated heteromonocyclic group” include oxiranyl group, thiolanyl group, aziridinyl group, azetidinyl group, oxetanyl group, pyrrolidinyl group, pyrrolidino group (1-pyrrolidinyl group), tetrahydrofuranyl group, tetrahydrothienyl group, oxazolinyl group, oxazolidinyl group, isoxazolinyl group, isoxazolidinyl group, thiazolinyl group, thiazolidinyl group, isothiazolinyl group, isothiazolidinyl group, imidazolinyl group, imidazolidinyl group, pyrazolinyl group, pyrazolidinyl group, piperidinyl group, piperidino group (1-piperidinyl group), morpholinyl group, morpholino group (4-morpholinyl group), thiomorpholinyl group, thio morpholino group 4-thiomorpholinyl group)piperazinyl group, piperazino group (1-piperazinyl group), hexahydro-1,3-oxazinyl group, homomorpholine, and homopiperazine.

The term “unsaturated heteromonocyclic group” refers to monocyclic group which has an unsaturated bond, contains the same or different 1 to 4 hetero atoms selected from nitrogen atom, oxygen atom or sulfur atom besides carbon atom, and is 5- to 6-membered.

Examples of “unsaturated heteromonocyclic group” include furyl group, thienyl group, pyrrolyl group, oxazolyl group, isoxazolyl group, thiazolyl group, isothiazolyl group, imidazolyl group, pyrazolyl group, oxadiazolyl group (1,2,5-oxadiazolyl group, 1,3,4-oxadiazolyl group, 1,2,4-oxadiazolyl group), thiadiazolyl group (1,2,5-thiadiazolyl group, 1,3,4-thiadiazolyl group, 1,2,4-thiadiazolyl group), triazolyl group (1,2,3-triazolyl group, 1,2,4-triazolyl group), tetrazolyl group, pyridyl group, pyrimidinyl group, pyridazinyl group, pyrazinyl group, and triazinyl group.

Preferred examples of “unsaturated heteromonocyclic group” include thienyl group, oxazolyl group, thiazolyl group, imidazolyl group, pyrazolyl group, oxadiazolyl group (1,3,4-oxadiazolyl group, 1,2,4-oxadiazolyl group), triazolyl group (1,2,4-triazolyl group), tetrazolyl group, pyridyl group, and pyrimidinyl group.

The term “unsaturated fused heterocyclic group” refers to unsaturated fused heterocyclic group which has an unsaturated bond, contains the same or different 1 to 4 hetero atoms selected from nitrogen atom, oxygen atom or sulfur atom besides carbon atom, and is 8- to 10-membered.

Examples of “unsaturated fused heterocyclic group” include quinolyl group, benzofuranyl group, benzothienyl group, indolyl group, imidazole[1,2-a]pyridyl group, and [1,2,4]triazolo[4,3-a]pyridinyl group.

The term “saturated fused heterocyclic group” refers to saturated fused heterocyclic group which contains the same or different 1 to 4 hetero atoms selected from nitrogen atom, oxygen atom or sulfur atom besides carbon atom, and is 8- to 10-membered.

Examples of “saturated fused heterocyclic group” include octahydroindolyl group.

The term “ring P” is “monocyclic heterocycle which is a carboxylic acid equivalent wherein the carboxylic acid equivalent may optionally be substituted”. The “ring P” includes the following:

Examples of a compound wherein “cyclic moiety W is selected from the following (1) to (3):

(1) pyrrolidinyl,

(2) piperidinyl,

(3) C₃₋₇ cycloalkyl”

include the following:

wherein nw is an integer selected from 0 or 1 to 4.

The term “autoimmune disease” is a collective term for diseases which relate to conditions wherein own immune system excessively reacts to own healthy cells or tissues and attacks them, and includes for example, rheumatoid arthritis, psoriasis, inflammatory bowel disease such as Crohn's disease and ulcerative colitis, multiple sclerosis, systemic lupus erythematosus, Behcet's disease, sarcoidosis, Harada disease, ankylosing spondylitis, uveitis, polymyalgia rheumatica, type I diabetes.

The “allergic disease” refers to a disease due to an excessive immune response to a particular antigen, and includes for example, atopic dermatitis, allergic rhinitis such as pollinosis, allergic conjunctivitis, allergic gastroenteritis, bronchial asthma, childhood asthma, food allergy, drug allergy, hives and the like.

The term “metabolic disease” refers to a disease caused by abnormal metabolic turnover or a disease relating to metabolic abnormality, and includes for example, diabetes such as type I diabetes and type II diabetes.

The “RORγ antagonist” refers to a compound which can inhibit a function of retinoid-related orphan receptor y (RORγ) to make the activity thereof disappear or reduced.

Examples of each substituent in the compound represented by formula [I] are explained as follows.

In one aspect,

is

A compound represented by Formula [I] wherein

is

is a compound represented by the following formula:

A compound represented by Formula [I] wherein

is

is a compound represented by the following formula:

In one aspect,

is monocyclic heteroaromatic group wherein the monocyclic heteroaromatic ring comprises the same or different 1 to 4 hetero atoms selected from nitrogen atom, oxygen atom, or sulfur atom besides carbon atom, and, for example is 5- to 7-membered.

Examples of

include furyl group, thienyl group, pyrrolyl group, oxazolyl group, isoxazolyl group, thiazolyl group, isothiazolyl group, imidazolyl group, pyrazolyl group, oxadiazolyl group oxadiazolyl group, 1,3,4-oxadiazolyl group, 1,2,4-oxadiazolyl group), thiadiazolyl group (1,2,5-thiadiazolyl group, 1,3,4-thiadiazolyl group, 1,2,4-thiadiazolyl group), triazolyl group (1,2,3-triazolyl group, 1,2,4-triazolyl group), tetrazolyl group, pyridyl group, pyrimidinyl group, pyridazinyl group, and pyrazinyl group, and triazinyl, and preferred examples include:

The more preferred examples of

include:

and the further preferred examples include:

In one aspect, each R^{a1} is the same or different and selected from:

(1) C₁₋₆ alkyl group which may optionally be substituted with the same or different 1 to 5 substituents selected from Group A,

(2) halogen atom, or

(3) C₃₋₇ cycloalkyl group which may optionally be substituted with the same or different 1 to 5 substituents selected from Group A.

Examples of R^{a1} which is C₁₋₆ alkyl group which may optionally be substituted with the same or different 1 to 5 substituents selected from Group A include C₁₋₆ alkyl group (such as methyl group, ethyl group, propyl group, isopropyl group, butyl group, isobutyl group, sec-butyl group, tert-butyl group, pentyl group, isopentyl group, neopenty group, tert-pentyl group, 1-ethylpropyl group, hexyl group, isohexyl group, 1,1-dimethylbutyl group, 2,2-dimethylbutyl group, 3,3-dimethylbutyl group, 2-ethylbutyl group, and 1,1-dimethyl-2-methylpropyl group), which may optionally be substituted with the same or different 1 to 5 substituents selected from the group consisting of halogen atom such as fluorine atom and C₃₋₇ cycloalkyl group wherein said C₃₋₇ cycloalkyl group may optionally be substituted with the same or different 1 to 5 substituents selected from the group consisting of C₁₋₆ alkyl group such as CH₃— and halogen atom.

Preferred examples of R^{a1} include CH₃—, (CH₃)₂CHCH₂—, (CH₃)₃C—, (CH₃)₂CH(CH₂)₂—, (CH₃)₃CCH₂—, (CH₃)₃C(CH₂)₂—, CF₃—, CF₃(CH₂)₂—, (CH₃)₂CHCF₂—, CF₃C(CH₃)₂—, (CH₃)₂CHCH₂CF₂—, (CH₃)₃CCF₂—, (CH₃)₃CCH₂CF₂—,

Examples of R^{a1} which is halogen atom include fluorine atom, chlorine atom, bromine atom, and iodine atom, and the preferred examples include chlorine atom.

Examples of R^{a1} which is C₃₋₇ cycloalkyl group which may optionally be substituted with the same or different 1 to 5 substituents selected from Group A include cyclopropyl group, cyclobutyl group, cyclopentyl group, cyclohexyl group, and cycloheptyl group, each of which may optionally be substituted with the same or different 1 substituents selected from the group consisting of C₁₋₆ alkyl group such as (CH₃)₃CCH₂—, (CH₃)₂CHCH₂—, and CH₃— and halogen atom such as fluorine atom;

Preferred examples of said R^{a1} include

In one aspect, R^{b} is:

(1) C₁₋₆ alkyl group which may optionally be substituted with the same or different 1 to 5 halogen atoms, or

(2) C₃₋₇ cycloalkyl group.

Examples of R^{b} which is C₁₋₆ alkyl group which may optionally be substituted with the same or different 1 to 5 halogen atoms include C₁₋₃ alkyl group which may optionally be substituted with the same or different 1 to 3 halogen atoms such as fluorine. Preferred examples of R^{b} include CF₃—, CHF₂—, and CH₃CF₂—.

Examples of R^{b} which is C₃₋₇ cycloalkyl group include cyclopropyl group, cyclobutyl group, cyclopentyl group, cyclohexyl group, and cycloheptyl group, and preferred examples include cyclopropyl group.

In one aspect, R^{c} is

(1) hydrogen atom, or

(2) C₁₋₆ alkyl group.

In one preferred aspect, R^{c} is hydrogen atom.

Preferred examples of R^{c} which is C₁₋₆ alkyl group include CH₃—, CH₃CH₂—, and (CH₃)₃CCH₂—.

In one aspect, each R^{d} is the same or different and selected from

(1) halogen atom, or

(2) C₁₋₆ alkyl group which may optionally be substituted with the same or different 1 to 5 halogen atoms.

Preferred examples of R^{d} which is halogen atom include fluorine atom and chlorine atom.

Examples of R^{d} which is C₁₋₆ alkyl group which may optionally be substituted with the same or different 1 to 5 halogen atoms include CH₃— and CH₃CH₂—, and preferred examples include CH₃—.

In one aspect, R^{e} is hydrogen atom.

In one aspect, n^{a} is an integer selected from 0 or 1 to 3.

Preferred examples of n^{a} include 1 and 2.

In one aspect, n^{c} is an integer selected from 0 or 1 to 3.

Preferred examples of n^{c} include 1, 2, and 3.

In one aspect, n^{d} is an integer selected from 0 or 1 to 3.

Preferred examples of n^{d} include 1.

In one aspect, m is an integer selected from 0 or 1 to 5.

Preferred examples of m include 2 and 3.

In one aspect, Group A is

(a) C₁₋₆ alkyl group,

(b) halogen atom,

(c) C₃₋₇ cycloalkyl group which may optionally be substituted with the same or different 1 to 5 substituents selected from the group consisting of C₁₋₆ alkyl group and halogen atom.

In Group A, preferred examples of C₁₋₆ alkyl group include methyl group, ethyl group, propyl group, isopropyl group, butyl group, isobutyl group, sec-butyl group, tert-butyl group, pentyl group, isopentyl group, neopenty group, tert-pentyl group, 1-ethylpropyl group, hexyl group, isohexyl group, 1,1-dimethylbutyl group, 2,2-dimethylbutyl group, 3,3-dimethylbutyl group, 2-ethylbutyl group, and 1,1-dimethyl-2-methylpropyl group; and the more preferred examples include (CH₃)₃CCH₂—, (CH₃)₂CHCH₂—, and CH₃—.

In Group A, examples of halogen atom include fluorine atom, chlorine atom, bromine atom, and iodine atom, and preferred examples include fluorine atom.

In Group A, examples of C₃₋₇ cycloalkyl group which may optionally be substituted with the same or different 1 to 5 substituents selected from the group consisting of C₁₋₆ alkyl group and halogen atom include cyclopropyl group, cyclobutyl group, cyclopentyl group, and cyclohexyl group, each of which may optionally be substituted with the same or different 1 to 5 C₁₋₆ alkyl groups such as CH₃—; and specific examples include

The preferred aspects of a compound represented by Formula [I] include compounds of the following formulas.

Each symbol in each formula is as defined in [01].

Other preferred aspects of a compound represented by Formula [I] include compounds of the following formulas:

TABLE 1

TABLE 2

TABLE 3

Examples of each substituent in the compounds represented by the general formulas described in [101] to [117], [139] to [142] are explained as follows.

is unsaturated heteromonocyclic group selected from the following (i) to (v):

Examples of

which is substituted with R^{a} and R^{b} include

In one aspect, R^{a} is selected from the following (1) to (12):

(1) C₁₋₁₂ alkyl group which may optionally be substituted with the same or different 1 to 5 substituents selected from Group A,

(2) C₃₋₇ cycloalkyl group which may optionally be substituted with the same or different 1 to 5 substituents selected from Group A,

(3) C₂₋₁₂ alkenyl group which may optionally be substituted with the same or different 1 to 5 substituents selected from Group A,

(4) C₂₋₁₂ alkynyl group which may optionally be substituted with the same or different 1 to 5 substituents selected from Group A,

(5) C₅₋₁₁ spirocyclic cycloalkyl group which may optionally be substituted with the same or different 1 to 5 substituents selected from Group A,

(6) cross-linked C₅₋₁₂ cycloalkyl group which may optionally be substituted with the same or different 1 to 5 substituents selected from Group A,

(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,

(8) phenyl group which may optionally be substituted with the same or different 1 to 5 substituents selected from Group A,

(9) C₉₋₁₀ fused carbocyclic group which may optionally be substituted with the same or different 1 to 5 substituents selected from Group A,

(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(11) saturated fused heterocyclic group which may optionally be substituted with the same or different 1 to 5 substituents selected from Group A wherein the saturated heteromonocyclic group has an unsaturated bond, comprises the same or different 1 to 4 hetero atoms selected from nitrogen atom, oxygen atom, or sulfur atom besides carbon atom, and is 8 to 10-membered,

(12) unsaturated fused heterocyclic group which may optionally be substituted with the same or different 1 to 5 substituents selected from Group A wherein the unsaturated heteromonocyclic group has an unsaturated bond, comprises the same or different 1 to 4 hetero atoms selected from nitrogen atom, oxygen atom, or sulfur atom besides carbon atom, and is 8 to 10-membered.

In one aspect, examples of “C₁₋₁₂ alkyl group which may optionally be substituted with the same or different 1 to 5 substituents selected from Group A” include:

In one aspect, examples of “C₃₋₇ cycloalkyl group which may optionally be substituted with the same or different 1 to 5 substituents selected from Group A” include cyclopropyl group, cyclobutyl group, cyclopentyl group, cyclohexyl group, cycloheptyl group,

In one aspect, examples of “C₂₋₁₂ alkenyl group which may optionally be substituted with the same or different 1 to 5 substituents selected from Group A” include

In one aspect, examples of “C₂₋₁₂ alkynyl group which may optionally be substituted with the same or different 1 to 5 substituents selected from Group A” include

In one aspect, examples of “C₅₋₁₁ spirocyclic cycloalkyl group which may optionally be substituted with the same or different 1 to 5 substituents selected from Group A” include

In one aspect, examples of “cross-linked C₅₋₁₂ cycloalkyl group which may optionally be substituted with the same or different 1 to 5 substituents selected from Group A” include

In one aspect, examples of “saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered” include

and the examples further include

In one aspect, examples of “phenyl group which may optionally be substituted with the same or different 1 to 5 substituents selected from Group A” include

In one aspect, examples of “C₉₋₁₀ fused carbocyclic group which may optionally be substituted with the same or different 1 to 5 substituents selected from Group A” include

In one aspect, examples of “unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered” include furyl group, thienyl group, pyrrolyl group, oxazolyl group, isoxazolyl group, thiazolyl group, isothiazolyl group, imidazolyl group, pyrazolyl group, oxadiazolyl group (1,2,5-oxadiazolyl group, 1,3,4-oxadiazolyl group, 1,2,4-oxadiazolyl group), thiadiazolyl group (1,2,5-thiadiazolyl group, 1,3,4-thiadiazolyl group, 1,2,4-thiadiazolyl group), triazolyl group (1,2,3-triazolyl group, 1,2,4-triazolyl group), tetrazolyl group, pyridyl group, pyrimidinyl group, pyridazinyl group, pyrazinyl group, and triazinyl group, and the examples further include

and further the examples include

In one aspect, examples of “saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered” include

In one aspect, examples of “unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered” include quinolyl group, benzofuranyl group, benzothienyl group, indolyl group, imidazole[1,2-a]pyridyl group, and [1,2,4]triazolo[4,3-a]pyridinyl group, and the examples further include

In a preferred aspect, R^{a} is selected from the following (1) to (3):

(1) C₁₋₁₂ alkyl group,

(2) cyclobutyl group substituted with C₁₋₇ alkyl group,

(3) phenyl group substituted with alkyl group.
R^{b} is selected from the following (1) to (6):

(1) C₁₋₆ alkyl group which may optionally be substituted with the same or different 1 to 5 halogen atoms,

(2) C₃₋₇ cycloalkyl group which may optionally be substituted with the same or different 1 to 5 substituents selected from the group consisting of halogen atom and C₁₋₆ alkyl group,

(3) —CH═CH—C(═O)—OR^{bb1},

(4) —CH₂—CH₂—C(═O)—OR^{bb2},

(5) —CH₂—O—CH₂—C(═O)—OR^{bb3},

(6) hydrogen atom.
R^{bb1}, R^{bb2}, and R^{bb3} are each independently hydrogen atom or C₁₋₆ alkyl group.

In one aspect, examples of “C₁₋₆ alkyl group which may optionally be substituted with the same or different 1 to 5 halogen atoms” include

In one aspect, examples of “C₃₋₇ cycloalkyl group which may optionally be substituted with the same or different 1 to 5 substituents selected from the group consisting of halogen atom and C₁₋₆ alkyl group” include

In one aspect, examples of “—CH═CH—C(═O)—OR^{bb1}” include

### —CH═CH—C(═O)—OH,

### —CH═CH—C(═O)—OCH₃.

In one aspect, examples of “—CH₂—CH₂—C(═O)—OR^{bb2}” include

—CH₂—CH₂—C(═O)—OH,
—CH₂—CH₂—C(═O)—OCH₃.

In one aspect, examples of “—CH₂—O—CH₂—C(═O)—OR^{bb3}” include

—CH₂—O—CH₂—C(═O)—OH,
—CH₂—O—CH₂—C(═O)—OCH₃.

In a preferred aspect, R^{b} is selected from the following (1) or (2):

(1) C₁₋₃ alkyl group which may optionally be substituted with the same or different 1 to 5 halogen atoms,

(2) cyclopropyl group.
R^{e} is hydrogen atom or C₁₋₆ alkyl group, preferably “hydrogen atom”.

Each R^{j} is the same or different C₁₋₁₂ alkyl group which may optionally be substituted with the same or different 1 to 5 substituents selected from Group A, and examples of R^{j} include

Q is selected from the following (1) to (9):

(1) phenyl group,

(2) C₃₋₇ cycloalkyl group,

(3) C₉₋₁₀ fused carbocyclic group,

(4) cross-linked C₅₋₁₂ cycloalkyl group,

(5) C₃₋₈ alkyl group,

(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,

(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,

(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered.

In one aspect, examples of “C₃₋₇ cycloalkyl group” include

In one aspect, examples of “C₉₋₁₀ fused carbocyclic group” include

In one aspect, examples of “cross-linked C₅₋₁₂ cycloalkyl group” include

In one aspect, examples of “C₃₋₈ alkyl group” include

In one aspect, examples of “saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered” include

In one aspect, examples of “unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered” include quinolyl group, benzofuranyl group, benzothienyl group, indolyl group, imidazole[1,2-a]pyridyl group, and [1,2,4]triazolo[4,3-a]pyridinyl group, and the examples further include

In one aspect, examples of “unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered” include

and the examples further include

In one aspect, examples of “saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered” include

Preferably, Q is phenyl group.

Examples of the moiety:

include

and the examples further include

Y is selected from the following (1) to (3):

(1) single bond,

(2) —S(═O)₂—,

(3) alkylene which may optionally be substituted with 1 to 3 hydroxyl groups.

Examples of said “C₁₋₃ alkylene which may optionally be substituted with 1 to 3 hydroxyl groups” include

Preferred examples of Y include “single bond”.

m is each independently an integer selected from 0 or 1 to 5, preferably “0, 1, 2, or 3”.

n^{j} is each independently 0, 1 or 2, preferably “2”.

Examples of each substituent in the compounds represented by the general formulas described in [102] and [139] are explained as follows.

Each R^{w} is the same or different and selected from the following (1) to (17):

(1) C₁₋₁₂ alkyl group which may optionally be substituted with the same or different 1 to 5 substituents selected from the group consisting of hydroxyl group and halogen atom,

(2) unsaturated heteromonocyclic group which may optionally be substituted with the same or different 1 to 5 C₁₋₆ alkyl groups wherein the unsaturated heteromonocyclic ring has an unsaturated bond, comprises the same or different 1 to 4 hetero atoms selected from nitrogen atom, oxygen atom, or sulfur atom besides carbon atom, and is 5 to 7-membered,

(3) —(CH₂)_{wn1}—C(═O)—OR^{ww1},

(4) —(CH₂)_{wn1}—C(═O)—(CH₂)_{wn1}—NR^{ww2}R^{ww3},

(5) —(CH₂)_{wn1}—C(═O)—(CH₂)_{wn1}—C(═O)—OR^{ww4},

(6) —NR^{ww5}R^{ww6},

(7) —OR^{ww7},

(8) —C(═O)—R^{ww8},

(9) —S(═O)₂—R^{ww9},

(10) ═O,

(11) methylene,

(12) —(CH₂)_{wn2}-ring P,

(13) halogen atom,

(14) —NR^{ww10}—C(═O)—R^{ww11},

(15) —NR^{ww12}—C(═O)—NR^{ww13}R^{ww14},

(16) —NR^{ww15}—S(═O)₂—R^{ww16},

(17) —NR^{ww17}—S(═O)₂—NR^{ww18}R^{ww19};

In one aspect, examples of “C₁₋₁₂ alkyl group which may optionally be substituted with the same or different 1 to 5 substituents selected from the group consisting of hydroxyl group and halogen atom” include

In one aspect, examples of “unsaturated heteromonocyclic group which may optionally be substituted with the same or different 1 to 5 C₁₋₆ alkyl groups wherein the unsaturated heteromonocyclic ring has an unsaturated bond, comprises the same or different 1 to 4 hetero atoms selected from nitrogen atom, oxygen atom, or sulfur atom besides carbon atom, and is 5 to 7-membered” include

In one aspect, examples of “—(CH₂)_{wn2}-ring P” include

Cyclic moiety W is selected from the following (1) to (3)

(1) pyrrolidinyl,

(2) piperidinyl,

(3) C₃₋₇ cycloalkyl,

preferably, pyrrolidinyl, piperidinyl, or, C₃₋₆ cycloalkyl.

cn is each independently an integer selected from 0 or 1 to 3, preferably 0, 1 or 2.

In Formula [III-X-C], examples of the moiety

include

wherein nw is an integer selected from 0 or 1 to 4, and the examples further include

Examples of each substituent in the compounds represented by the general formulas described in [103] to [107], and [141] are explained as follows.

R^{c} is selected from the following (1) to (17):

(1) —(CH₂)ₙ₁—C(═O)—OR^{cc1},

(2) —O—(CH₂)ₙ₂—C(═O)—OR^{cc2},

(3) —(CH₂)ₙ₃-ring P,

(4) —(CH₂)ₙ₄—C(═O)—NH—S(═O)₂—CH₃,

(5) C₁₋₆ alkyl group which may optionally be substituted with the same or different 1 to 5 substituents selected from Group C,

(6) C₃₋₆ cycloalkyl group which may optionally be substituted with the same or different 1 to 5 substituents selected from Group C,

(7) C₂₋₁₂ alkenyl group,

(8) C₂₋₁₂ alkynyl group,

(9) —NR^{cc3}R^{cc4},

(10) —OR^{cc5},

(11) —O—CH₂CH₂—OH,

(12) —O—CH₂C(═O)NH—CH₃,

In one aspect, examples of “—(CH₂)ₙ₁—C(═O)—OR^{cc1}” include

### —CH₂—C(═O)—OH,

—CH₂—C(═O)—OCH₃,
—(CH₂)₂—C(═O)—OH,
—(CH₂)₂—C(═O)—OCH₃,
—(CH₂)₃—C(═O)—OH,
—(CH₂)₃—C(═O)—OCH₃.

In one aspect, examples of “—O—(CH₂)ₙ₂—C(═O)—OR^{cc2}” include

### —O—CH₂—C(═O)—OH,

—O—CH₂—C(═O)—OCH₃,
—O—(CH₂)₂—C(═O)—OH,
—O—(CH₂)₂—C(═O)—OCH₃.

In one aspect, examples of “—(CH₂)ₙ₃-ring P” include

In one aspect, examples of “—(CH₂)ₙ₄—C(═O)—NH—S(═O)₂—CH₃” include

—CH₂—C(═O)—NH—S(═O)₂—CH₃,
—(CH₂)₂—C(═O)—NH—S(═O)₂—CH₃,
—(CH₂)₃—C(═O)—NH—S(═O)₂—CH₃.

In one aspect, examples of “C₁₋₆ alkyl group which may optionally be substituted with the same or different 1 to 5 substituents selected from Group C” include

—(CH₂)₂—OH,
—(CH₂)₃—OH,
—(CH₂)₄—OH,

In one aspect, examples of “C₃₋₆ cycloalkyl group which may optionally be substituted with the same or different 1 to 5 substituents selected from Group C” include

In one aspect, examples of “—NR^{cc3}R^{cc4}” include

### —NH₂,

—N(CH₃)₂,

### —NH(CH₃),

### —NH—C(═O)—CH₃,

—N(CH₃)—C(═O)—CH₂.

In one aspect, examples of “—OR^{cc5}” include

### —OH,

### —O—CH₃,

### —O—CH₂-Ph.

R^{f} is hydrogen atom or C₁₋₆ alkyl group, preferably hydrogen atom or methyl group.

Examples of each substituent in the compounds represented by the general formula described in [108] are explained as follows.

Each R^{au} is the same or different and selected from the following (1) or (2):

(1) C₇₋₁₂ alkyl group,

(2) C₁₋₁₂ alkyl group which is substituted with the same or different 1 to 5 substituents selected from Group AU.

Examples of “C₁₋₁₂ alkyl group which is substituted with the same or different 1 to 5 substituents selected from Group AU” include

Cyclic moiety UU is selected from the following (1) or (2):

(1) C₃₋₇ cycloalkyl group,

(2) phenyl group.

Examples of “C₃₋₇ cycloalkyl group” include

n^{u} is an integer selected from 0 or 1 to 3, preferably “1”.

Preferred aspects of a compound described in [101] include compounds of the following formulas.

Each symbol in each formula is as defined in [101].

```
TABLE 4
Aspects
  [III-X-B]
  [III-F-B]
  [III-H-B]
  [III-G-B]
  [III-J-B]
  [III-K-B]
  [III-L-B]
```

Other preferred aspects include the following compounds.

```
TABLE 5
Table 101-2
  [III-X-B-QB]
  [III-F-B-QB]
  [III-H-B-QB]
  [III-G-B-QB]
  [III-J-B-QB]
  [III-K-B-QB]
  [III-L-B-QB]
```

Preferred examples of the combination of substituents in Tables 101-1 and 101-2 are shown below:

TABLE 6
Aspect 101-3

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) C₃₋₇ cycloalkyl group which may optionally be |
| | substituted with the same or different 1 to 5 |
| | substituents selected from Group A, |
| | (3) phenyl group which may optionally be substituted |
| | with the same or different 1 to 5 substituents |
| | selected from Group A, |
| R^{b} | selected from the following (1) or (2): |
| | (1) C₁₋₆ alkyl group which may optionally be |
| | substituted with the same or different 1 to 5 halogen |
| | atoms, |
| | (2) C₃₋₇ cycloalkyl group which may optionally be |
| | substituted with the same or different 1 to 5 substituents |
| | selected from the group consisting of halogen atom |
| | and C₁₋₆ alkyl group, |
| m | an integer selected from 0 or 1 to 4 |
| R^{d} | selected from the following (1) to (3): |
| | (1) halogen atom, |
| | (2) C₁₋₆ alkyl group which may optionally be |
| | substituted with the same or different 1 to 5 |
| | substituents selected from Group B, |
| | (3) C₃₋₇ cycloalkyl group which may optionally be |
| | substituted with the same or different 1 to 5 |
| | substituents selected from Group B, |

TABLE 7
Aspect 101-4

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) cyclobutyl group which may optionally be |
| | substituted with the same or different 1 to 5 |
| | substituents selected from Group A, |
| | (3) phenyl group which may optionally be substituted |
| | with the same or different 1 to 5 substituents |
| | selected from Group A, |
| R^{b} | selected from the following (1) or (2): |
| | (1) C₁₋₃ alkyl group which may optionally be |
| | substituted with the same or different 1 to 5 |
| | halogen atoms, |
| | (2) cyclopropyl group which may optionally be |
| | substituted with the same or different 1 to 5 |
| | substituents selected from the group consisting |
| | of halogen atom and C₁₋₆ alkyl group, |
| m | an integer selected from 0 or 1 to 3 |
| R^{d} | selected from the following (1) to (3): |
| | (1) halogen atom, |
| | (2) C₁₋₃ alkyl group which may optionally be |
| | substituted with the same or different 1 to 5 |
| | substituents selected from Group B, |
| | (3) cyclopropyl group which may optionally be |
| | substituted with the same or different 1 to 5 |
| | substituents selected from Group B, |

TABLE 8
Aspect 101-5

| | |
|---|---|
| R^{a} | selected from the following (1) to (3): |
| | (1) C₁₋₁₂ alkyl group, |
| | (2) cyclobutyl group substituted with C₁₋₇ alkyl |
| | group, |
| | (3) phenyl group substituted with C₁₋₇ alkyl group, |
| R^{b} | selected from the following (1) or (2): |
| | (1) C₁₋₃ alkyl group which may optionally be |
| | substituted with the same or different 1 to 5 halogen |
| | atoms, |
| | (2) cyclopropyl group, |
| m | an integer selected from 0 or 1 to 3 |
| R^{d} | selected from the following (1) to (3): |
| | (1) halogen atom, |
| | (2) C₁₋₃ alkyl group, |
| | (3) cyclopropyl group, |

Other preferred examples of the combination of substituents in Tables 101-1 and 101-2 are shown below:

TABLE 9
Aspect 101-6

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) C₃₋₇ cycloalkyl group which may optionally be |
| | substituted with the same or different 1 to 5 |
| | substituents selected from Group A, |
| | (3) phenyl group which may optionally be substituted |
| | with the same or different 1 to 5 substituents |
| | selected from Group A, |
| R^{b} | selected from the following (1) to (3): |
| | (1) —CH═CH—C(═O)—OR^{bb1}, |
| | (2) —CH₂—CH₂—C(═O)—OR^{bb2}, |
| | (3) —CH₂—O—CH₂—C(═O)—OR^{bb3} |
| m | an integer selected from 0 or 1 to 4 |
| R^{d} | selected from the following (1) to (3): |
| | (1) halogen atom, |
| | (2) C₁₋₆ alkyl group which may optionally be |
| | substituted with the same or different 1 to 5 |
| | substituents selected from Group B, |
| | (3) C₃₋₇ cycloalkyl group which may optionally be |
| | substituted with the same or different 1 to 5 |
| | substituents selected from Group B, |

TABLE 10
Aspect 101-7

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) cyclobutyl group which may optionally be |
| | substituted with the same or different 1 to 5 |
| | substituents selected from Group A, |
| | (3) phenyl group which may optionally be substituted |
| | with the same or different 1 to 5 substituents selected |
| | from Group A, |
| R^{b} | selected from the following (1) to (3): |
| | (1) —CH═CH—C(═O)—OR^{bb1}, |
| | (2) —CH₂—CH₂—C(═O)—OR^{bb2}, |
| | (3) —CH₂—O—CH₂—C(═O)—OR^{bb3} |
| m | an integer selected from 0 or 1 to 3 |
| R^{d} | selected from the following (1) to (3): |
| | (1) halogen atom, |
| | (2) C₁₋₃ alkyl group which may optionally be |
| | substituted with the same or different 1 to 5 |
| | substituents selected from Group B, |
| | (3) cyclopropyl group which may optionally be substituted |
| | with the same or different 1 to 5 substituents |
| | selected from Group B, |

TABLE 11
Aspect 101-8

| | |
|---|---|
| R^{a} | selected from the following (1) to (3): |
| | (1) C₁₋₁₂ alkyl group, |
| | (2) cyclobutyl group substituted with C₁₋₇ alkyl |
| | group, |
| | (3) phenyl group substituted with C₁₋₇ alkyl group, |
| R^{b} | selected from the following (1) to (3): |
| | (1) —CH═CH—C(═O)—OR^{bb1}, |
| | (2) —CH₂—CH₂—C(═O)—OR^{bb2}, |
| | (3) —CH₂—O—CH₂—C(═O)—OR^{bb3} |
| m | an integer selected from 0 or 1 to 3 |
| R^{d} | selected from the following (1) to (3): |
| | (1) halogen atom, |
| | (2) C₁₋₃ alkyl group, |
| | (3) cyclopropyl group, |

Preferred aspects of [101] include the following compounds:

TABLE 12

Preferred aspects of a compound described in [102] includes compounds of the following formulas.

Each symbol in each formula is as defined in [102].

```
TABLE 13
Table 102-1
  [III-X-C]
  [III-F-C]
  [III-H-C]
  [III-G-C]
  [III-J-C]
  [III-K-C]
  [III-L-C]
```

Other preferred aspects include the following compounds.

```
TABLE 14
Table 102-2
  [III-X-CUD-N5-QB]
  [III-F-CUD-N5-QB]
  [III-H-CUD-N5-QB]
  [III-G-CUD-N5-QB]
  [III-J-CUD-N5-QB]
  [III-K-CUD-N5-QB]
  [III-L-CUD-N5-QB]
```

Other preferred aspects include the following compounds.

```
TABLE 15
Table 102-3
  [III-X-CUD-CNW-QB]
  [III-F-CUD-CNW-QB]
  [III-H-CUD-CNW-QB]
  [III-G-CUD-CNW-QB]
  [III-J-CUD-CNW-QB]
  [III-K-CUD-CNW-QB]
  [III-L-CUD-CNW-QB]
```

In Table 102-3, nw is an integer selected from 0 or 1 to 4, preferably 0 or 1 to 3.

Other preferred aspects include the following compounds.

```
TABLE 16
Table 102-4
  [III-X-CUD-C5-QB]
  [III-F-CUD-C5-QB]
  [III-H-CUD-C5-QB]
  [III-G-CUD-C5-QB]
  [III-J-CUD-C5-QB]
  [III-K-CUD-C5-QB]
  [III-L-CUD-C5-QB]
```

Preferred examples of the combination of substituents in Tables 102-1, 102-2, 102-3, 102-4 are shown below.

TABLE17
Aspect 102-5

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) C₃₋₇ cycloalkyl group which may optionally be |
| | substituted with the same or different 1 to 5 |
| | substituents selected from Group A, |
| | (3) phenyl group which may optionally be substituted |
| | with the same or different 1 to 5 substituents |
| | selected from Group A, |
| R^{b} | selected from the following (1) or (2): |
| | (1) C₁₋₆ alkyl group which may optionally be |
| | substituted with the same or different 1 to 5 halogen |
| | atoms, |
| | (2) C₃₋₇ cycloalkyl group which may optionally be sub- |
| | stituted with the same or different 1 to 5 substituents |
| | selected from the group consisting of halogen atom and |
| | C₁₋₆ alkyl group, |
| R^{w} | each Rw is the same or different and selected from the |
| | following (1) to (12): |
| | (1) C₁₋₁₂ alkyl group which may optionally be |
| | substituted with the same or different 1 to 5 |
| | substituents selected from the group consisting of |
| | hydroxyl group and halogen atom, |
| | (2) unsaturated heteromonocyclic group which may |
| | optionally be substituted with the same or different 1 |
| | to 5 C₁₋₆ alkyl groups wherein the unsaturated |
| | heteromonocyclic ring has an unsaturated bond, |
| | comprises the same or different 1 to 4 hetero atoms |
| | selected from nitrogen atom, oxygen atom, or sulfur |
| | atom besides carbon atom, and is 5 to 7-membered, |
| | (3) —(CH₂)_{wn1}—C(═O)—OR^{ww1}, |
| | (4) —(CH₂)_{wn1}—C(═O)—(CH₂)_{wn1}—NR^{ww2}R^{ww3}, |
| | (5) —(CH₂)_{wn1}—C(═O)—(CH₂)_{wn1}—C(═O)—OR^{ww4}, |
| | (6) —NR^{ww5}R^{ww6}, |
| | (7) —OR^{ww7}, |
| | (8) —C(═O)—R^{ww8}, |
| | (9) —S(═O)₂—R^{ww9}, |
| | (10) ═O, |
| | (11) methylene, |
| | (12) —(CH₂)_{wn2}-ring P, |
| m | an integer selected from 0 or 1 to 4 |
| R^{d} | selected from the following (1) to (3): |
| | (1) halogen atom, |
| | (2) C₁₋₆ alkyl group which may optionally be |
| | substituted with the same or different 1 to 5 |
| | substituents selected from Group B, |
| | (3) C₃₋₇ cycloalkyl group which may optionally be |
| | substituted with the same or different 1 to 5 |
| | substituents selected from Group B, |

TABLE 18
Aspect 102-6

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) cyclobutyl group which may optionally be |
| | substituted with the same or different 1 to 5 |
| | substituents selected from Group A, |
| | (3) phenyl group which may optionally be substituted |
| | with the same or different 1 to 5 substituents selected |
| | from Group A, |
| R^{b} | selected from the following (1) or (2): |
| | (1) C₁₋₃ alkyl group which may optionally be |
| | substituted with the same or different 1 to 5 halogen |
| | atoms, |
| | (2) cyclopropyl group which may optionally be substituted |
| | with the same or different 1 to 5 substituents |
| | selected from the group consisting of halogen atom and |
| | C₁₋₆ alkyl group, |
| R^{w} | each Rw is the same or different and selected from the |
| | following (1) to (12): |
| | (1) C₁₋₁₂ alkyl group which may optionally be |
| | substituted with the same or different 1 to 5 |
| | substituents selected from the group consisting of |
| | hydroxyl group and halogen atom, |
| | (2) unsaturated heteromonocyclic group which may |
| | optionally be substituted with the same or different 1 |
| | to 5 C₁₋₆ alkyl groups wherein the unsaturated |
| | heteromonocyclic ring has an unsaturated bond, |
| | comprises the same or different 1 to 4 hetero atoms |
| | selected from nitrogen atom, oxygen atom, or sulfur |
| | atom besides carbon atom, and is 5 to 7-membered, |
| | (3) —(CH₂)_{wn1}—C(═O)—OR^{ww1}, |
| | (4) —(CH₂)_{wn1}—C(═O)—(CH₂)_{wn1}—NR^{ww2}R^{ww3}, |
| | (5) —(CH₂)_{wn1}—C(═O)—(CH₂)_{wn1}—C(═O)—OR^{ww4}, |
| | (6) —NR^{ww5}R^{ww6}, |
| | (7) —OR^{ww7}, |
| | (8) —C(═O)—R^{ww8}, |
| | (9) —S(═O)₂—R^{ww9}, |
| | (10) ═O, |
| | (11) —(CH₂)_{wn2}-ring P, |
| m | an integer selected from 0 or 1 to 3 |
| R^{d} | selected from the following (1) to (3): |
| | (1) halogen atom, |
| | (2) C₁₋₃ alkyl group which may optionally be |
| | substituted with the same or different 1 to 5 |
| | substituents selected from Group B, |
| | (3) cyclopropyl group which may optionally be substituted |
| | with the same or different 1 to 5 substituents selected |
| | from Group B, |

TABLE 19
Aspect 102-7

| | |
|---|---|
| R^{a} | selected from the following (1) to (3): |
| | (1) C₁₋₁₂ alkyl group, |
| | (2) cyclobutyl group substituted with C₁₋₇ alkyl |
| | group, |
| | (3) phenyl group substituted with C₁₋₇ alkyl group, |
| R^{b} | selected from the following (1) or (2): |
| | (1) C₁₋₃ alkyl group which may optionally be |
| | substituted with the same or different 1 to 5 halogen |
| | atoms, |
| | (2) cyclopropyl group, |
| R^{w} | each Rw is the same or different and selected from the |
| | following (1) to (7): |
| | (1) —(CH₂)_{wn1}—C(═O)—OR^{ww1}, |
| | (2) —(CH₂)_{wn1}—C(═O)—(CH₂)_{wn1}—C(═O)—OR^{ww4}, |
| | (3) —NR^{ww5}R^{ww6}, |
| | (4) —OR^{ww7}, |
| | (5) —C(═O)—R^{ww8}, |
| | (6) —S(═O)₂—R^{ww9}, |
| | (7) —(CH₂)_{wn2}-ring 2, |
| m | an integer selected from 0 or 1 to 3 |
| R^{d} | selected from the following (1) to (3): |
| | (1) halogen atom, |
| | (2) C₁₋₃ alkyl group, |
| | (3) cyclopropyl group, |

Preferred aspects of [102] include the following compounds:

TABLE 20

Preferred aspects of a compound described in [103] include compounds of the following formulas.

Each symbol in each formula is as defined in [103].

```
TABLE 21
Table 103-1
  [III-X-D2]
  [III-F-D2]
  [III-H-D2]
  [III-G-D2]
  [III-J-D2]
  [III-K-D2]
  [III-L-D2]
```

Other preferred aspects include the following compounds.

```
TABLE 22
Table 103-2
  [III-X-D2-U-QB]
  [III-F-D2-U-QB]
  [III-H-D2-U-QB]
  [III-G-D2-U-QB]
  [III-X-D2-D-QB]
  [III-J-D2-U-QB]
  [III-K-D2-U-QB]
  [III-L-D2-U-QB]
  [III-J-D2-D-QB]
  [III-F-D2-D-QB]
  [III-H-D2-D-QB]
  [III-G-D2-D-QB]
  [III-K-D2-D-QB]
  [III-L-D2-D-QB]
```

Preferred examples of the combination of substituents in Tables 103-1 and 103-2 are shown below.

TABLE 23
Aspect 103-3

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) C₃₋₇ cycloalkyl group which may optionally be |
| | substituted with the same or different 1 to 5 |
| | substituents selected from Group A, |
| | (3) phenyl group which may optionally be substituted |
| | with the same or different 1 to 5 substituents |
| | selected from Group A, |
| R^{b} | selected from the following (1) or (2): |
| | (1) C₁₋₆ alkyl group which may optionally be |
| | substituted with the same or different 1 to 5 halogen |
| | atoms, |
| | (2) C₃₋₇ cycloalkyl group which may optionally be |
| | substituted with the same or different 1 to 5 substituted |
| | selected from the group consisting of halogen atom and |
| | C₁₋₆ alkyl group, |
| R^{c} | R^{c} is selected from the following (1) to (4): |
| | (1) —(CH₂)ₙ₁—C(═O)—OR^{cc1}, |
| | (2) —O—(CH₂)ₙ₂—C(═O)—OR^{cc2}, |
| | (3) —(CH₂)ₙ₃-ring P, |
| | (4) —(CH₂)ₙ₄—C(═O)—NH—S(═O)₂—CH₃ |
| m | an integer selected from 0 or 1 to 4 |
| R^{d} | selected from the following (1) to (3): |
| | (1) halogen atom, |
| | (2) C₁₋₆ alkyl group which may optionally be |
| | substituted with the same or different 1 to 5 |
| | substituents selected from Group B, |
| | (3) C₃₋₇ cycloalkyl group which may optionally be |
| | substituted with the same or different 1 to 5 |
| | substituents selected from Group B, |

TABLE 24
Aspect 103-4

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) cyclobutyl group which may optionally be |
| | substituted with the same or different 1 to 5 |
| | substituents selected from Group A, |
| | (3) phenyl group which may optionally be substituted |
| | with the same or different 1 to 5 substituents selected |
| | from Group A, |
| R^{b} | selected from the following (1) or (2): |
| | (1) C₁₋₃ alkyl group which may optionally be |
| | substituted with the same or different 1 to 5 halogen |
| | atoms, |
| | (2) cyclopropyl group which may optionally be substituted |
| | with the same or different 1 to 5 substituents selected |
| | from the group consisting of halogen atom and C₁₋₆ |
| | alkyl group, |
| R^{c} | R^{c} is selected from the following (1) to (4): |
| | (1) —(CH₂)ₙ₁—C(═O)—OR^{cc1} |
| | (2) —O—(CH₂)ₙ₂—C(═O)—OR^{cc2}, |
| | (3) —(CH₂)ₙ₃-ring P, |
| | (4) —(CH₂)ₙ₄—C(═O)—NH—S(═O)₂—CH₃ |
| m | an integer selected from 0 or 1 to 3 |
| R^{d} | selected from the following (1) to (3): |
| | (1) halogen atom, |
| | (2) C₁₋₃ alkyl group which may optionally be |
| | substituted with the same or different 1 to 5 |
| | substituents selected from Group B, |
| | (3) cyclopropyl group which may optionally be substituted |
| | with the same or different 1 to 5 substituents selected |
| | from Group B, |

TABLE 25
Aspect 103-5

| | |
|---|---|
| R^{a} | selected from the following (1) to (3): |
| | (1) C₁₋₁₂ alkyl group, |
| | (2) cyclobutyl group substituted with C₁₋₇ alkyl |
| | group, |
| | (3) phenyl group substituted with C₁₋₇ alkyl group, |
| R^{b} | selected from the following (1) or (2): |
| | (1) C₁₋₃ alkyl group which may optionally be |
| | substituted with the same or different 1 to 5 halogen |
| | atoms, |
| | (2) cyclopropyl group, |
| R^{c} | R^{c} is selected from the following (1) to (4): |
| | —(CH₂)ₙ₁—C(═O)—OR^{cc1} wherein R^{cc1} is hydrogen atom, |
| | —O—(CH₂)ₙ₂—C(═O)—OR^{cc2} wherein R^{cc2} is hydrogen atom, |
| | (3) —(CH₂)ₙ₃-ring P, |
| | (4) —(CH₂)ₙ₄—C(═O)—NH—S(═O)₂—CH₃ |
| m | an integer selected from 0 or 1 to 3 |
| R^{d} | selected from the following (1) to (3): |
| | (1) halogen atom, |
| | (2) C₁₋₃ alkyl group, |
| | (3) cyclopropyl group, |

Preferred aspects of a compound described in [103] include the following compounds:

TABLE 26

Preferred aspects of a compound described in [104], [105] and [106] include compounds of the following formulas. Each symbol in each of the following formulas is as defined in [104].

```
TABLE 27
Table 104-1
  [III-X-D1]
  [III-F-D1]
  [III-H-D1]
  [III-G-D1]
  [III-J-D1]
  [III-K-D1]
  [III-L-D1]
```

```
TABLE 28
Table 104-2
  [III-X-D1-D-QB]
  [III-J-D1-D-QB]
  [III-F-D1-D-QB]
  [III-H-D1-D-QB]
  [III-K-D1-D-QB]
  [III-G-D1-D-QB]
  [III-L-D1-D-QB]
  [III-X-D1-U-QB]
  [III-J-D1-U-QB]
  [III-F-D1-U-QB]
  [III-H-D1-U-QB]
  [III-K-D1-U-QB]
  [III-G-D1-U-QB]
  [III-L-D1-U-QB]
```

```
TABLE 29
Table 104-3
  [III-X-D12]
  [III-F-D12]
  [III-H-D12]
  [III-K-D12]
  [III-G-D12]
  [III-L-D12]
```

```
TABLE 30
Table 104-4
  [III-X-D1-D-RCP-QB]
  [III-J-D1-D-RCP-QB]
  [III-F-D1-D-RCP-QB]
  [III-H-D1-D-RCP-QB]
  [III-K-D1-D-RCP-QB]
  [III-G-D1-D-RCP-QB]
  [III-L-D1-D-RCP-QB]
```

Preferred examples of the combination of substituents in Tables 104-1, 104-2, 104-3, and 104-4 are shown below.

TABLE 31
Aspect 104-5

| | |
|---|---|
| R^{a} | selected from the following (1) to (4): |
| | (1) C₁₋₁₂ alkyl group which may optionally be |
| | substituted with the same or different 1 to 5 |
| | substituents selected from Group A, |
| | (2) C₃₋₇ cycloalkyl group which may optionally be |
| | substituted with the same or different 1 to 5 |
| | substituents selected from Group A, |
| | (3) phenyl group which may optionally be substituted |
| | with the same or different 1 to 5 substituents |
| | selected from Group A, |
| | (4) unsaturated heteromonocyclic group which may optionally |
| | be substituted with the same or different 1 to 5 |
| | substituents selected from Group A wherein the unsaturated |
| | heteromonocyclic ring has an unsaturated bond, comprises |
| | the same or different 1 to 4 hetero atoms selected |
| | from nitrogen atom, oxygen atom, or sulfur atom |
| | besides carbon atom, and is 5 to 6-membered, |
| R^{b} | selected from the following (1) or (2): |
| | (1) C₁₋₆ alkyl group which may optionally be |
| | substituted with the same or different 1 to 5 halogen |
| | atoms, |
| | (2) C₃₋₇ cycloalkyl group which may optionally be substituted |
| | with the same or different 1 to 5 substituents |
| | selected from the group consisting of halogen atom and |
| | C₁₋₆ alkyl group, |
| R^{c} | R^{c} is selected from the following (1) to (4): |
| | (1) —(CH₂)ₙ₁—C(═O)—OR^{cc1}, |
| | (2) —O—(CH₂)ₙ₂—C(═O)—OR^{cc2}, |
| | (3) —(CH₂)ₙ₃-ring P, |
| | (4) —(CH₂)ₙ₄—C(═O)—NH—S(═O)₂—CH₃ |
| m | an integer selected from 0 or 1 to 4 |
| R^{d} | selected from the following (1) to (3): |
| | (1) halogen atom, |
| | (2) C₁₋₆ alkyl group which may optionally be |
| | substituted with the same or different 1 to 5 |
| | substituents selected from Group B, |
| | (3) C₃₋₇ cycloalkyl group which may optionally be |
| | substituted with the same or different 1 to 5 |
| | substituents selected from Group B, |

TABLE 32
Aspect 104-6

| | |
|---|---|
| R^{a} | selected from the following (1) to (4): |
| | (1) C₁₋₁₂ alkyl group which may optionally be |
| | substituted with the same or different 1 to 5 |
| | substituents selected from Group A, |
| | (2) cyclobutyl group which may optionally be |
| | substituted with the same or different 1 to 5 |
| | substituents selected from Group A, |
| | (3) phenyl group which may optionally be substituted |
| | with the same or different 1 to 5 substituents |
| | selected from Group A, |
| | (4) unsaturated heteromonocyclic group which may optionally |
| | be substituted with the same or different 1 to 5 |
| | substituents selected from Group A wherein the unsaturated |
| | heteromonocyclic ring has an unsaturated bond, |
| | comprises the same or different 1 to 4 hetero atoms |
| | selected from nitrogen atom, oxygen atom, or sulfur atom |
| | besides carbon atom, and is 5 to 6-membered, |
| R^{b} | selected from the following (1) or (2): |
| | (1) C₁₋₃ alkyl group which may optionally be |
| | substituted with the same or different 1 to 5 halogen |
| | atoms, |
| | (2) cyclopropyl group which may optionally be |
| | substituted with the same or different 1 to 5 substituents |
| | selected from the group consisting of halogen atom and |
| | C₁₋₆ alkyl group, |
| R^{c} | R^{c} is selected from the following (1) to (4): |
| | (1) —(CH₂)ₙ₁—C(═O)—OR^{cc1}, |
| | (2) —O—(CH₂)ₙ₂—C(═O)—OR^{cc2}, |
| | (3) —(CH₂)ₙ₃-ring P, |
| | (4) —(CH₂)ₙ₄—C(═O)—NH—S(═O)₂—CH₃ |
| m | an integer selected from 0 or 1 to 3 |
| R^{d} | selected from the following (1) to (3): |
| | (1) halogen atom, |
| | (2) C₁₋₃ alkyl group which may optionally be |
| | substituted with the same or different 1 to 5 |
| | substituents selected from Group B, |
| | (3) C₃ cycloalkyl group which may optionally be |
| | substituted with the same or different 1 to 5 substituents |
| | selected from Group B, |

TABLE 33
Aspect 104-7

| | |
|---|---|
| R^{a} | selected from the following (1) to (4): |
| | (1) C₁₋₁₂ alkyl group, |
| | (2) cyclobutyl group substituted with C₁₋₇ alkyl |
| | group, |
| | (3) phenyl group substituted with C₁₋₇ alkyl group, |
| | (4) unsaturated heteromonocyclic group substituted with |
| | C₁₋₇ alkyl group wherein the unsaturated heteromonocyclic |
| | ring has an unsaturated bond, comprises the same |
| | or different 1 to 4 hetero atoms selected from nitrogen |
| | atom, oxygen atom, or sulfur atom besides carbon atom, |
| | and is 5 to 6-membered, |
| R^{b} | selected from the following (1) or (2): |
| | (1) C₁₋₃ alkyl group which may optionally be |
| | substituted with the same or different 1 to 5 halogen |
| | atoms, |
| | (2) cyclopropyl group, |
| R^{c} | R^{c} is selected from the following (1) to (4): |
| | —(CH₂)ₙ₁—C(═O)—OR^{cc1} wherein R^{cc1} is hydrogen atom, |
| | —O—(CH₂)ₙ₂—C(═O)—OR^{cc2} wherein R^{cc2} is hydrogen atom, |
| | (3) —(CH₂)ₙ₃-ring P, |
| | (4) —(CH₂)ₙ₄—C(═O)—NH—S(═O)₂—CH₃ |
| m | an integer selected from 0 or 1 to 3 |
| R^{d} | selected from the following (1) to (3): |
| | (1) halogen atom, |
| | (2) C₁₋₃ alkyl group, |
| | (3) cyclopropyl group, |

### Aspect 104-8:

In Aspects 104-5, 104-6, and 104-7, preferred examples of R^{f} include hydrogen atom.

### Aspect 104-9:

In Aspects 104-5, 104-6, and 104-7, preferred examples of R^{f} includes C₁₋₃ alkyl group.

### Aspect 104-10:

In Aspects 104-5, 104-6, 104-7, 104-8, and 104-9, preferred examples of “unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered” for the substituent R^{a} include

In preferred aspects, the following compounds are included.

TABLE 34

TABLE 35

TABLE 36

In preferred aspects, the following compounds are included.

TABLE 37

TABLE 38

In preferred aspects, the following compounds are included.

TABLE 39

In preferred aspects, the following compounds are included.

TABLE 40

Preferred aspects of a compound described in [107] include compounds of the following formulas. Each symbol in each formula is as defined in [107].

```
TABLE 41
Table 107-1
  [III-X-D1-D2H]
  [III-X-D1-D-QB]
  [III-K-D1-D-QB]
  [III-G-D1-D-QB]
  [III-X-D1-U-QB]
  [III-K-D1-U-QB]
  [III-G-D1-U-QB]
```

Preferred examples of the combination of substituents in the above Table 107-1 are shown below.

TABLE 42
Aspect 107-2

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) C₃₋₇ cycloalkyl group which may optionally be |
| | substituted with the same or different 1 to 5 |
| | substituents selected from Group A, |
| | (3) phenyl group which may optionally be substituted |
| | with the same or different 1 to 5 substituents selected |
| | from Group A, |
| R^{b} | selected from the following (1) or (2): |
| | (1) C₁₋₆ alkyl group which may optionally be |
| | substituted with the same or different 1 to 5 halogen |
| | atoms, |
| | (2) C₃₋₇ cycloalkyl group which may optionally be |
| | substituted with the same or different 1 to 5 substituents |
| | selected from the group consisting of halogen atom |
| | and C₁₋₆ alkyl group, |
| R^{c} | selected from the following (1) to (3): |
| | (1) —(CH₂)ₙ₃-ring P, |
| | (2) —(CH₂)ₙ₄—C(═O)—NH—S(═O)₂—CH₃, |
| | (3) C₁₋₆ alkyl group which may optionally be substituted |
| | with the same or different 1 to 5 substituents |
| | selected from Group C, |
| m | an integer selected from 0 or 1 to 4 |
| R^{d} | selected from the following (1) to (3): |
| | (1) halogen atom, |
| | (2) C₁₋₆ alkyl group which may optionally be |
| | substituted with the same or different 1 to 5 |
| | substituents selected from Group B, |
| | (3) C₃₋₇ cycloalkyl group which may optionally be |
| | substituted with the same or different 1 to 5 |
| | substituents selected from Group B, |

TABLE 43
Aspect 107-3

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) cyclobutyl group which may optionally be |
| | substituted with the same or different 1 to 5 |
| | substituents selected from Group A, |
| | (3) phenyl group which may optionally be substituted |
| | with the same or different 1 to 5 substituents selected |
| | from Group A, |
| R^{b} | selected from the following (1) or (2): |
| | (1) C₁₋₃ alkyl group which may optionally be |
| | substituted with the same or different 1 to 5 halogen |
| | atoms, |
| | (2) cyclopropyl group which may optionally be substituted |
| | with the same or different 1 to 5 substituents |
| | selected from the group consisting of halogen atom and |
| | C₁₋₆ alkyl group, |
| R^{c} | selected from the following (1) to (3): |
| | (1) —(CH₂)ₙ₃-ring P, |
| | (2) —(CH₂)ₙ₄—C(═O)—NH—S(═O)₂—CH₃, |
| | (3) C₁₋₆ alkyl group which may optionally be substituted |
| | with the same or different 1 to 5 substituents |
| | selected from Group C, |
| m | an integer selected from 0 or 1 to 3 |
| R^{d} | selected from the following (1) to (3): |
| | (1) halogen atom, |
| | (2) C₁₋₃ alkyl group which may optionally be |
| | substituted with the same or different 1 to 5 |
| | substituents selected from Group B, |
| | (3) cyclopropyl group which may optionally be substituted |
| | with the same or different 1 to 5 substituents |
| | selected from Group B, |

TABLE 44
Aspect 107-4

| | |
|---|---|
| R^{a} | selected from the following (1) to (3): |
| | (1) C₁₋₁₂ alkyl group, |
| | (2) cyclobutyl group substituted with C₁₋₇ alkyl |
| | group, |
| | (3) phenyl group substituted with C₁₋₇ alkyl group, |
| R^{b} | selected from the following (1) or (2): |
| | (1) C₁₋₃ alkyl group which may optionally be |
| | substituted with the same or different 1 to 5 halogen |
| | atoms, |
| | (2) cyclopropyl group, |
| R^{c} | selected from the following (1) to (3): |
| | (1) —(CH₂)ₙ₃-ring P, |
| | (2)—(CH₂)ₙ₄—C(═O)—NH—S(═O)₂—CH₃, |
| m | an integer selected from 0 or 1 to 3 |
| R^{d} | selected from the following (1) to (3): |
| | (1) halogen atom, |
| | (2) C₁₋₃ alkyl group, |
| | (3) cyclopropyl group, |

### Aspect 107-5:

In Aspects 107-2, 107-3, 107-4, ring P is selected from the following heterocyclic groups:

```
TABLE 45
Table 107-6
  [III-X-D1-D-RCP-QB]
  [III-K-D1-D-RCP-QB]
  [III-G-D1-D-RCP-QB]
```

Preferred examples of the combination of substituents in Tables 107-6 are shown below.

TABLE 46
Aspect 107-7

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) C₃₋₇ cycloalkyl group which may optionally be |
| | substituted with the same or different 1 to 5 |
| | substituents selected from Group A, |
| | (3) phenyl group which may optionally be substituted |
| | with the same or different 1 to 5 substituents |
| | selected from Group A, |
| R^{b} | selected from the following (1) or (2): |
| | (1) C₁₋₆ alkyl group which may optionally be |
| | substituted with the same or different 1 to 5 halogen |
| | atoms, |
| | (2) C₃₋₇ cycloalkyl group which may optionally be |
| | substituted with the same or different 1 to 5 |
| | substituents selected from the group consisting |
| | of halogen atom and C₁₋₆ alkyl group, |
| m | an integer selected from 0 or 1 to 4 |
| R^{d} | selected from the following (1) to (3): |
| | (1) halogen atom, |
| | (2) C₁₋₆ alkyl group which may optionally be |
| | substituted with the same or different 1 to 5 |
| | substituents selected from Group B, |
| | (3) C₃₋₇ cycloalkyl group which may optionally be |
| | substituted with the same or different 1 to 5 |
| | substituents selected from Group B, |

TABLE 47
Aspect 107-8

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) cyclobutyl group which may optionally be |
| | substituted with the same or different 1 to 5 |
| | substituents selected from Group A, |
| | (3) phenyl group which may optionally be |
| | substituted with the same or different 1 to 5 |
| | substituents selected from Group A, |
| R^{b} | selected from the following (1) or (2): |
| | (1) C₁₋₃ alkyl group which may optionally be |
| | substituted with the same or different 1 to 5 |
| | halogen atoms, |
| | (2) cyclopropyl group which may optionally be |
| | substituted with the same or different 1 to 5 |
| | substituents selected from the group consisting |
| | of halogen atom and C₁₋₆ alkyl group, |
| m | an integer selected from 0 or 1 to 3 |
| R^{d} | selected from the following (1) to (3): |
| | (1) halogen atom, |
| | (2) C₁₋₃ alkyl group which may optionally be |
| | substituted with the same or different 1 to 5 |
| | substituents selected from Group B, |
| | (3) cyclopropyl group which may optionally be |
| | substituted with the same or different 1 to 5 |
| | substituents selected from Group B, |

TABLE 48
Aspect 107-9

| | |
|---|---|
| R^{a} | selected from the following (1) to (3): |
| | (1) C₁₋₁₂ alkyl group, |
| | (2) cyclobutyl group substituted with C₁₋₇ |
| | alkyl group, |
| | (3) phenyl group substituted with C₁₋₇ alkyl |
| | group, |
| R^{b} | selected from the following (1) or (2): |
| | (1) C₁₋₃ alkyl group which may optionally be |
| | substituted with the same or different 1 to 5 |
| | halogen atoms, |
| | (2) cyclopropyl group, |
| m | an integer selected from 0 or 1 to 3 |
| R^{d} | selected from the following (1) to (3): |
| | (1) halogen atom, |
| | (2) C₁₋₃ alkyl group, |
| | (3) cyclopropyl group, |

### Aspect 107-10:

In Aspects 107-7, 107-8, and 107-9, ring P is selected from the following heterocyclic groups:

Preferred aspects of a compound described in [107] include the following compounds:

TABLE 49

TABLE 50

Preferred aspects of a compound described in [108] include compounds of the following formulas.

Each symbol in each formula is as defined in [108].

Other preferred aspects include the following compounds.

```
TABLE 51
Table 108-1
  [III-X-D11] [III-X-D11]
  [III-X-D11] [III-K-D11]
  [III-X-D11] [III-G-D11]
  [III-K-D11-D-QB]
  [III-G-D11-D-QB]
  [III-K-D11-U-QB]
  [III-G-D11-U-QB]
```

Preferred examples of the combination of substituents in Table 108-1 are shown below.

TABLE 52
Aspect 108-2

| | |
|---|---|
| R^{au} | the same or different and selected from the |
| | following (1) or (2): |
| | (1) C₇₋₁₂ alkyl group, |
| | (2) C₁₋₁₂ alkyl group which is substituted with the |
| | same or different 1 to 5 substituents selected from |
| | Group AU; |
| R^{b} | selected from the following (1) or (2): |
| | (1) C₁₋₆ alkyl group which may optionally be |
| | substituted with the same or different 1 to 5 |
| | halogen atoms, |
| | (2) C₃₋₇ cycloalkyl group which may optionally be |
| | substituted with the same or different 1 to 5 |
| | substituents selected from the group consisting of |
| | halogen atom and C₁₋₆ alkyl group, |
| R^{c} | selected from the following (1) or (2): |
| | (1) —(CH₂)ₙ₁—C(═O)—OR^{cc1} wherein R^{cc1} is hydrogen |
| | atom, |
| | (2) —O—(CH₂)ₙ₂—C(═O)—OR^{cc2} wherein R^{cc2} is hydrogen |
| | atom, |
| R^{d} | the same or different and selected from the |
| | following (1) to (3): |
| | (1) halogen atom, |
| | (2) C₁₋₆ alkyl group which may optionally be |
| | substituted with the same or different 1 to 5 |
| | substituents selected from Group B, |
| | (3) C₃₋₇ cycloalkyl group which may optionally be |
| | substituted with the same or different 1 to 5 |
| | substituents selected from Group B, |
| m | each independently, an integer selected from 0 or 1 |
| | to 4; |

### Aspect 108-3:

In Aspect 108-2, Group AU consists of the following (a) to (c):

(a) —OR^{AU1},

(b) —C(═O)—OR^{AU2},

(c) —C(═O)—NR^{AU3}R^{AU4};

wherein R^{AU1}, R^{AU2}, R^{AU3}, and R^{AU4} are each independently, hydrogen atom or C₁₋₆ alkyl group.

TABLE 53
Aspect 108-4

| R^{au} | the same or different and selected from the |
|---|---|
| | following (1) or (2): |
| | (1) C₇₋₁₂ alkyl group, |
| | (2) C₁₋₁₂ alkyl group which is substituted with the |
| | same or different 1 to 5 substituents selected from |
| | Group AU; |
| cyclic | C₃₋₇ cycloalkyl group |
| moiety | |
| UU | |
| R^{b} | selected from the following (1) or (2): |
| | (1) C₁₋₃ alkyl group which may optionally be |
| | substituted with the same or different 1 to 5 |
| | halogen atoms, |
| | (2) cyclopropyl group, |
| R^{c} | selected from the following (1) or (2): |
| | (1) —(CH₂)ₙ₁—C(═O)—OR^{cc1} wherein R^{cc1} is hydrogen |
| | atom, |
| | (2) —O—(CH₂)ₙ₂—C(═O)—OR^{cc2} wherein R^{cc2} is hydrogen |
| | atom, |
| R^{d} | the same or different and selected from the |
| | following (1) to (3): |
| | (1) halogen atom, |
| | (2) C₁₋₆ alkyl group, |
| | (3) C₃₋₇ cycloalkyl group, |
| m | an integer selected from 0 or 1 to 3; |
| n^{u} | 1 |

### Aspect 108-5:

In Aspects 108-4, Group AU consists of the following (a) to (c):

(a) —OR^{AU1},

(b) —C(═O)—OR^{AU2},

(c) —C(═O)—NR^{AU3}R^{AU4};

wherein R^{AU1}, R^{AU2}, R^{AU3}, R^{AU4} are each independently, hydrogen atom or C₁₋₆ alkyl group.

In other preferred aspects of a compound described in [108], the following are included.

Each symbol in each formula is as defined in [108].

```
TABLE 54
Table 108-6
  [III-UU-C-K-D11]
  [III-UU-C-G-D11]
  [III-UU-B-K-D11]
  [III-UU-B-G-D11]
```

```
TABLE 55
Table 108-7
  [III-UU-C-K-D11-QB]
  [III-UU-C-G-D11-QB]
  [III-UU-B-K-D11-QB]
  [III-UU-B-G-D11-QB]
  [III-UU-C-K-D11U-QB]
  [III-UU-C-G-D11U-QB]
  [III-RA-C-K-D11U-QB]
  [III-RA-C-G-D11U-QB]
```

Preferred examples of the combination of substituents in Tables 108-6 and 108-7 are shown below.

TABLE 56
Aspect 108-8

| | |
|---|---|
| R^{au} | selected from the following (1) or (2): |
| | (1) C₇₋₁₂ alkyl group, |
| | (2) C₁₋₁₂ alkyl group which is substituted with the |
| | same or different 1 to 5 substituents selected from |
| | Group AU; |
| R^{b} | selected from the following (1) or (2): |
| | (1) C₁₋₆ alkyl group which may optionally be |
| | substituted with the same or different 1 to 5 |
| | halogen atoms, |
| | (2) C₃₋₇ cycloalkyl group which may optionally be |
| | substituted with the same or different 1 to 5 |
| | substituents selected from the group consisting of |
| | halogen atom and C₁₋₆ alkyl group, |
| R^{c} | selected from the following (1) or (2): |
| | (1) —(CH₂)ₙ₁—C(═O)—OR^{cc1} wherein R^{cc1} is hydrogen |
| | atom, |
| | (2) —O—(CH₂)ₙ₂—C(═O)—OR^{cc2} wherein R^{cc2} is hydrogen |
| | atom, |
| R^{d} | the same or different and selected from the |
| | following (1) to (3): |
| | (1) halogen atom, |
| | (2) C₁₋₆ alkyl group which may optionally be |
| | substituted with the same or different 1 to 5 |
| | substituents selected from Group B, |
| | (3) C₃₋₇ cycloalkyl group which may optionally be |
| | substituted with the same or different 1 to 5 |
| | substituents selected from Group B, |
| m | each independently, an integer selected from 0 or 1 |
| | to 4;. |

### Aspect 108-9:

In the above aspect D108-8, Group AU consists of the following (a) to (b):

(a) —OR^{AU1},

(b) —C(═O)—OR^{AU2},

(c) —C(═O)—NR^{AU3}R^{AU4};

wherein R^{AU1}, R^{AU2}, R^{AU3}, R^{AU4} are each independently, hydrogen atom or C₁₋₆ alkyl group.

In other preferred aspects of a compound described in [108], the following are included.

Each symbol in each formula is as defined in [108].

```
TABLE 57
Table 108-10
  [III-UU-C-K-D11UC-QB]
  [III-UU-C-G-D11UC-QB]
  [III-RA-C-K-D11UC-QB]
  [III-RA-C-G-D11UC-QB]
```

### Aspect 108-11:

In Table 108-10, “R^{AU}” is selected from:

### Aspect 108-12

In Aspect 108-11, “R^{b}” is a group selected from the following:

(1) C₁₋₃ alkyl group which may optionally be substituted with the same or different 1 to 5 halogen atoms,

(2) cyclopropyl group.

In preferred aspects of a compound described in [108], the following compounds are included:

TABLE 58

Preferred aspects of a compound described in [109] include compounds of the following formulas.

Each symbol in each formula is as defined in [109].

```
TABLE 59
Table 109-1
  [III-X-D12]
  [III-K-D12]
  [III-G-D12]
  [III-K-D12-QB]
  [III-G-D12-QB]
```

Preferred examples of the combination of substituents in Table 109-1 are shown below.

TABLE 60
Aspect 109-2

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) C₃₋₇ cycloalkyl group which may optionally be |
| | substituted with the same or different 1 to 5 |
| | substituents selected from Group A, |
| | (3) phenyl group which may optionally be substituted |
| | with the same or different 1 to 5 substituents |
| | selected from Group A, |
| R^{c} | R^{c} is selected from the following (1) to (4): |
| | (1) —(CH₂)ₙ₁—C(═O)—OR^{cc1}, |
| | (2) —O—(CH₂)ₙ₂—C(═O)—OR^{cc2}, |
| | (3) —(CH₂)ₙ₃-ring P, |
| | (4) —(CH₂)ₙ₄—C(═O)—NH—S(═O)₂—CH₃ |
| m | an integer selected from 0 or 1 to 4 |
| R^{d} | selected from the following (1) to (3): |
| | (1) halogen atom, |
| | (2) C₁₋₆ alkyl group which may optionally be |
| | substituted with the same or different 1 to 5 |
| | substituents selected from Group B, |
| | (3) C₃₋₇ cycloalkyl group which may optionally be |
| | substituted with the same or different 1 to 5 |
| | substituents selected from Group B, |

TABLE 61
Aspect 109-3

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) cyclobutyl group which may optionally be |
| | substituted with the same or different 1 to 5 |
| | substituents selected from Group A, |
| | (3) phenyl group which may optionally be substituted |
| | with the same or different 1 to 5 substituents |
| | selected from Group A, |
| R^{c} | selected from the following (1) to (4): |
| | (1) —(CH₂)ₙ₁—C(═O)—OR^{cc1}, |
| | (2) —O—(CH₂)ₙ₂—C(═O)—OR^{cc2}, |
| | (3) —(CH₂)ₙ₃-ring P, |
| | (4 ) —(CH₂)ₙ₄—C(═O)—NH—S(═O)₂—CH₃ |
| m | an integer selected from 0 or 1 to 3 |
| R^{d} | selected from the following (1) to (3): |
| | (1) halogen atom, |
| | (2) C₁₋₃ alkyl group which may optionally be |
| | substituted with the same or different 1 to 5 |
| | substituents selected from Group B, |
| | (3) cyclopropyl group which may optionally be |
| | substituted with the same or different 1 to 5 |
| | substituents selected from Group B, |

TABLE 62
Aspect 109-4

| | |
|---|---|
| R^{a} | selected from the following (1) to (3): |
| | (1) C₁₋₁₂ alkyl group, |
| | (2) cyclobutyl group substituted with C₁₋₇ alkyl |
| | group, |
| | (3) phenyl group substituted with C₁₋₇ alkyl group, |
| R^{c} | selected from the following (1) to (4): |
| | (1) —(CH₂)ₙ₁—C(═O)—OR^{cc1} wherein R^{cc1} is hydrogen atom, |
| | (2) —O—(CH₂)ₙ₂—C(═O)—OR^{cc2} wherein R^{cc2} is hydrogen |
| | atom, |
| | (3) —(CH₂)ₙ₃-ring P, |
| | (4) —(CH₂)ₙ₄—C(═O)—NH—S(═O)₂—CH₃ |
| m | an integer selected from 0 or 1 to 3 |
| R^{d} | selected from the following (1) to (3): |
| | (1) halogen atom, |
| | (2) C₁₋₃ alkyl group, |
| | (3) cyclopropyl group, |

In Preferred aspects of a compound of [109], the following compounds are included:

TABLE 63

Preferred aspects of a compound described in [110] includes compounds of the following formulas.

Each symbol in each formula is as defined in [110].

```
TABLE 64
Table 110-1
  [III-X-D13]
  [III-K-D13]
  [III-G-D13]
```

Preferred examples of the combination of substituents in the above table are shown below.

TABLE 65
Aspect 110-2

| | |
|---|---|
| R^{a} | selected from the following (1) to (3): |
| | (1) C₁₋₁₂ alkyl group, |
| | (2) cyclobutyl group substituted with C₁₋₇ alkyl |
| | group, |
| | (3) phenyl group substituted with C₁₋₇ alkyl group, |
| R^{c} | selected from the following (1) to (4): |
| | (1) —(CH₂)ₙ₁—C(═O)—OR^{cc1} wherein R^{cc1} is hydrogen atom, |
| | (2) —O—(CH₂)ₙ₂—C(═O)—OR^{cc2} wherein R^{cc2} is hydrogen |
| | atom, |
| | (3) —(CH₂)ₙ₃-ring P, |
| | (4) —(CH₂)ₙ₄—C(═O)—NH—S(═O)₂—CH₃ |
| m | an integer selected from 0 or 1 to 3 |
| R^{d} | selected from the following (1) to (3): |
| | (1) halogen atom, |
| | (2) C₁₋₃ alkyl group, |
| | (3) cyclopropyl group, |

### Aspect 110-3

In Aspects 110-2, Q is phenyl group, and R^{e} is hydrogen atom.

Preferred aspects of a compound described in [111] includes compounds of the following formulas.

Each symbol in each formula is as defined in [111].

```
TABLE 66
Table 111-1
  [III-X-D1-D2H]
  [III-X-D1-D2H]
  [III-X-D1-D2H]
  [III-X-D1-D-D2H]
  [III-X-D1-D-D2H]
```

Preferred examples of the combination of substituents in the above table are shown below.

TABLE 67
Aspect 111-2

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) C₃₋₇ cycloalkyl group which may optionally be |
| | substituted with the same or different 1 to 5 |
| | substituents selected from Group A, |
| | (3) phenyl group which may optionally be substituted |
| | with the same or different 1 to 5 substituents |
| | selected from Group A, |
| R^{b} | selected from the following (1) or (2): |
| | (1) C₁₋₆ alkyl group which may optionally be |
| | substituted with the same or different 1 to 5 halogen |
| | atoms, |
| | (2) C₃₋₇ cycloalkyl group which may optionally be |
| | substituted with the same or different 1 to 5 |
| | substituents selected from the group consisting of |
| | halogen atom and C₁₋₆ alkyl group, |
| R^{c} | selected from the following (1) to (4): |
| | (1) —(CH₂)ₙ₁—C(═O)—OR^{cc1}, |
| | (2) —O—(CH₂)ₙ₂—C(═O)—OR^{cc2}, |
| | (3) —(CH₂)ₙ₃-ring P, |
| | (4) —(CH₂)ₙ₄—C(═O)—NH—S(═O)₂—CH₃ |
| m | an integer selected from 0 or 1 to 4 |
| R^{d} | selected from the following (1) to (3): |
| | (1) halogen atom, |
| | (2) C₁₋₆ alkyl group which may optionally be |
| | substituted with the same or different 1 to 5 |
| | substituents selected from Group B, |
| | (3) C₃₋₇ cycloalkyl group which may optionally be |
| | substituted with the same or different 1 to 5 |
| | substituents selected from Group B, |

TABLE 68
Aspect 111-3

| | |
|---|---|
| R^{a} | selected from the following (1) to (3): |
| | (1) C₁₋₁₂ alkyl group which may optionally be |
| | substituted with the same or different 1 to 5 |
| | substituents selected from Group A, |
| | (2) cyclobutyl group which may optionally be |
| | substituted with the same or different 1 to 5 |
| | substituents selected from Group A, |
| | (3) phenyl group which may optionally be |
| | substituted with the same or different 1 to 5 |
| | substituents selected from Group A, |
| R^{b} | selected from the following (1) or (2): |
| | (1) C₁₋₆ alkyl group which may optionally be |
| | substituted with the same or different 1 to 5 |
| | halogen atoms, |
| | (2) C₃₋₇ cycloalkyl group which may optionally |
| | be substituted with the same or different 1 to 5 |
| | substituents selected from the group consisting |
| | of halogen atom and C₁₋₆ alkyl group, |
| R^{c} | selected from the following (1) to (4): |
| | (1) —(CH₂)ₙ₁—C(═O)—OR^{cc1}, |
| | (2) —O—(CH₂)ₙ₂—C(═O)—OR^{cc2}, |
| | (3) —(CH₂)ₙ₃-ring P, |
| | (4) —(CH₂)ₙ₄—C(═O)—NH—S(═O)₂—CH₃ |
| m | an integer selected from 0 or 1 to 3 |
| R^{d} | selected from the following (1) to (3): |
| | (1) halogen atom, |
| | (2) C₁₋₃ alkyl group which may optionally be |
| | substituted with the same or different 1 to 5 |
| | substituents selected from Group B, |
| | (3) cyclopropyl group which may optionally be |
| | substituted with the same or different 1 to 5 |
| | substituents selected from Group B, |

TABLE 69
Aspect 111-4

| | |
|---|---|
| R^{a} | selected from the following (1) to (3): |
| | (1) C₁₋₁₂ alkyl group, |
| | (2) cyclobutyl group substituted with C₁₋₇ alkyl |
| | group, |
| | (3) phenyl group substituted with C₁₋₇ alkyl group, |
| R^{b} | selected from the following (1) or (2): |
| | (1) C₁₋₆ alkyl group which may optionally be |
| | substituted with the same or different 1 to 5 halogen |
| | atoms, |
| | (2) C₃₋₇ cycloalkyl group which may optionally be |
| | substituted with the same or different 1 to 5 |
| | substituents selected from the group consisting |
| | of halogen atom and C₁₋₆ alkyl group, |
| R^{c} | selected from the following (1) to (4): |
| | (1) —(CH₂)ₙ₁—C(═O)—OR^{cc1} wherein R^{cc1} is hydrogen atom, |
| | (2) —O—(CH₂)ₙ₂—C(═O)—OR^{cc2} wherein R^{cc2} is hydrogen |
| | atom, |
| | (3) —(CH₂)ₙ₃-ring P, |
| | (4) —(CH₂)ₙ₄—C(═O)—NH—S(═O)₂—CH₃ |
| m | an integer selected from 0 or 1 to 3 |
| R^{d} | selected from the following (1) to (3): |
| | (1) halogen atom, |
| | (2) C₁₋₃ alkyl group, |
| | (3) cyclopropyl group, |

```
TABLE 70
Table 111-5
  [III-K-D1-D2H-NY]
  [III-G-D1-D2H-NY]
  [III-K-D1-D-D2H]
  [III-G-D1-D-D2H]
```

Preferred examples of the combination of substituents in Table 111-5 are shown below.

TABLE 71
Aspect 111-6

| | |
|---|---|
| R^{a} | selected from the following (1) to (3): |
| | (1) C₁₋₁₂ alkyl group, |
| | (2) cyclobutyl group substituted with C₁₋₇ alkyl |
| | group, |
| | (3) phenyl group substituted with C₁₋₇ alkyl group, |
| R^{c} | selected from the following (1) to (4): |
| | (1) —(CH₂)ₙ₁—C(═O)—OR^{cc1} wherein R^{cc1} is hydrogen atom, |
| | (2) —O—(CH₂)ₙ₂—C(═O)—OR^{cc2} wherein R^{cc2} is hydrogen |
| | atom, |
| | (3) —(CH₂)ₙ₃-ring P, |
| | (4) —(CH₂)ₙ₄—C(═O)—NH—S(═O)₂—CH₃ |
| m | an integer selected from 0 or 1 to 3 |
| R^{d} | selected from the following (1) to (3): |
| | (1) halogen atom, |
| | (2) C₁₋₃ alkyl group, |
| | (3) cyclopropyl group, |

### Aspect 111-7

In Aspects 111-2, 111-3, 111-4, and 111-6,

Q is selected from the following (1) to (6):

(1) C₃₋₇ cycloalkyl group,

(2) C₉₋₁₀ fused carbocyclic group selected from the group consisting of indanyl group and 1,2,3,4-tetrahydronaphthyl group,

(3) cross-linked C₅₋₁₂ cycloalkyl group,

(4) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,

(5) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,

(6) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered

(7) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered.

### Aspect 111-8:

In Aspects 111-2, 111-3, 111-4, and 111-6,

Q is selected from the following (1) to (6):

(1) C₃₋₇ cycloalkyl group selected from the following:

(2) indanyl group and 1,2,3,4-tetrahydronaphthyl group;

(3) cross-linked C₅₋₁₂ cycloalkyl group selected from the following:

(4) saturated heteromonocyclic group selected from the following:

(5) unsaturated fused heterocyclic group selected from the following:

(6) unsaturated heteromonocyclic group selected from the following:

In Preferred aspects of [111], the following compounds are included:

TABLE 72

TABLE 73

“Pharmaceutically acceptable salts” may be any nontoxic salt of the present invention compound, for example, include salts formed with inorganic acid, organic acid, inorganic base, organic base, amino acid and the like.

The inorganic acid salts include for example, salts formed with hydrochloric acid, nitric acid, sulfuric acid, phosphoric acid, hydrobromic acid and the like.

The organic acid salts include for example, salts formed with oxalic acid, maleic acid, citric acid, fumaric acid, lactic acid, malic acid, succinic acid, tartaric acid, acetic acid, trifluoroacetic acid, gluconic acid, ascorbic acid, methanesulfonic acid, benzenesulfonic acid, p-toluenesulfonic acid and the like.

The salts formed with inorganic base include for example, sodium salt, potassium salt, calcium salt, magnesium salt, ammonium salt and the like.

Preferred examples of the salts formed with inorganic base include sodium salt.

The salts formed with organic base include for example salts formed with methylamine, ethylamine, diethylamine, trimethylamine, triethylamine, ethanolamine, diethanolamine, triethanolamine, ethylenediamine, tris(hydroxymethyl)methylamine, cyclohexylamine, dicyclohexylamine, N,N′-dibenzyl ethylenediamine, guanidine, pyridine, picoline, choline, cinchonine, meglumine and the like.

The salts formed with amino acid include for example, salts formed with lysine, arginine, aspartic acid, glutamic acid and the like.

Hereinafter, “Formula [I] and the like” refers to the general formulas which are described herein such as Formula [I], [III-X-B], [III-J-B], [III-X-C], [III-J-C], [III-X-D2], [III-J-D2], [III-X-D1], [III-J-D1], [III-X-D1-D2H], [III-X-D11], and [III-X-D12].

Such salts can be formed by reacting compounds of Formula [I] and the like with inorganic base, organic base, inorganic acid, organic acid, or amino acid by conventional methods.

The term “solvate” refers to the compounds of Formula [I] and the like or pharmaceutically acceptable salts thereof which coordinate to the solvent molecules, and also includes hydrates. Such solvates are preferably pharmaceutically acceptable solvates. Such solvate includes for example hydrate, ethanol solvate, dimethyl sulfoxide solvate and the like of compounds of Formula [I] and the like or pharmaceutically acceptable salts thereof. The specific example includes hemihydrate, monohydrate, dihydrate or mono(ethanol)solvate of compounds of Formula [I] and the like or monohydrate of sodium salt of compounds of Formula [I] and the like, 2/3 (ethanol)solvate of dihydrochloride of the same and the like. Such solvates can be produced by conventional methods.

In addition, the compounds of Formula [I] and the like may have a variety of “isomer”. For example, the compounds can exist in E or Z forms or cis or trans isomers as geometric isomers. Moreover, the compounds which have asymmetric carbon atom(s) include enantiomers and diastereomers as stereoisomers according to said asymmetric carbon atom(s). Besides, the compounds which have axial chirality include stereoisomers according to said axial chirality. In some cases, tautomer may be included. Therefore, the present invention includes all of these isomers and mixtures thereof.

When a specific relative or absolute configuration, especially, of geometric isomer or of the asymmetric carbon atom of the alpha position from the 5-membered ring:

is not indicated in the structural formula such as Formula [I], except as otherwise provided, the compound of the structural formula may be any theoretically available stereoisomer, and may be a mixture thereof.

The preferred aspects of a compound represented by Formula [I] include compounds of the following formula:

When a compound of the present invention may exist as a tautomer, the compound of the present invention may be a single tautomer or a mixture of tautomers.

When a compound of the present invention has a carbon double bond, the compound of the present invention may be E form, Z form, or a mixture of E form and Z form.

When a compound of the present invention may exist as a stereoisomer which is recognized as cis/trans isomer, the compound of the present invention may be cis form, trans form, or a mixture of cis form and trans form.

When a compound of the present invention has one or more asymmetric carbons, the compound of the present invention may be a single enantiomer, a single diastereomer, a mixture of enantiomers and/or a mixture of diastereomers.

When a compound of the present invention may exist as an atropisomer, the compound of the present invention may exist as a single atropisomer or a mixture of atropisomers.

A compound of the present invention may have plurality structural features which may derive the above isomers. A compound of the present invention may comprise the above isomers in the any ratio.

When a general formula, a chemical structure, or a compound name is described herein without indicating stereochemistry, the compound includes the above possible isomers, unless otherwise designated.

A diastereomer mixture can be separated into each diastereomer by a conventional method such as chromatography and crystallization. Alternatively, a single diastereomer can be produced by using a stereochemically-single starting material or by synthesis process using a stereoselective reaction.

An enantiomer mixture can be separated into each enantiomer by using a method well-known in the art.

For example, an enantiomer mixture is reacted with a compound which is a substantially pure enantiomer (known as a chiral auxiliary) to give a diastereomer mixture, and then by using a standard technique such as fractionated crystallization or chromatography the diastereomer mixture can be separated into a single diastereomer which has a high isomeric ratio (i.e. substantially pure). The resulting single diastereomer is cleaved to remove the added chiral auxiliary to give an enantiomer of interest.

An enantiomer mixture of a compound can be directly separated by using a chromatography using a chiral stationary phase which is well-known in the art.

Further, either enantiomer of a compound can be obtained by using a substantially pure optically-active starting material, or by stereoselective synthesis (asymmetric induction) wherein a prochiral intermediate is treated with a chiral auxiliary or an asymmetric catalysis.

An absolute configuration can be determined by X-ray crystallographic analysis of a crystalline or an intermediate. The crystalline or the intermediate may be derivatized with a reagent having an asymmetric center whose configuration is known.

In addition, the compound represented by Formula [I] and the like may be labeled with one or more isotopes such as ³H, ¹⁴C, ³⁵S and the like. Besides, the compound represented by Formula [I] and the like also includes an isotopic compound thereof wherein one or more ¹H are replaced with ²H(D).

The compounds of Formula [I] and the like or pharmaceutically acceptable salts thereof are preferably purified to be substantively pure, more preferably 80% or purer.

According to the present invention, prodrugs of compounds of Formula [I] and the like may also be a useful medicine. The “prodrug” as used herein refers to derivatives of the present invention compound having a chemically or metabolically decomposable group, which show the inherent pharmaceutical activity upon hydrolysis, solvolysis, or other decompositions under physiological conditions in vivo, and may also be a complex connected with bonds other than covalent bonds or a salt. Prodrugs can be used for example, for improving absorption of oral administration or targeting the object site. A modified site includes highly reactive functional groups in the present invention compounds, such as hydroxyl group, carboxyl group, amino group, thiol group and the like.

The group that modifies the hydroxyl group includes specifically acetyl group, propionyl group, isobutyryl group, pivaloyl group, palmitoyl group, benzoyl group, 4-methylbenzoyl group, dimethylcarbamoyl group, dimethylaminomethylcarbonyl group, sulfo group, alanyl group, fumary group and the like. In addition, 3-(sodium carboxylate)benzoyl group, 2-(sodium carboxylate)ethylcarbonyl group and the like are also included.

The group that modifies the carboxyl group includes specifically methyl group, ethyl group, propyl group, isopropyl group, butyl group, isobutyl group, tert-butyl group, pivaloyloxymethyl group, carboxymethyl group, dimethylaminomethyl group, 1-(acetyloxy)ethyl group, 1-(ethoxycarbonyloxy)ethyl group, 1-(isopropyloxycarbonyloxy)ethyl group, 1-(cyclohexyloxycarbonyloxy)ethyl group, (5-methyl-2-oxo-1,3-dioxol-4-yl)methyl group, benzyl group, phenyl group, o-tolyl group, morpholinoethyl group, N,N-diethylcarbamoylmethyl group, phthalidyl group and the like.

The group that modifies the amino group includes specifically tert-butyl group, docosanoyl group, pivaloylmethyloxy group, alanyl group, hexylcarbamoyl group, pentylcarbamoyl group, 3-methylthio-1-(acetylamino)propylcarbonyl group, 1-sulfo-1-(3-ethoxy-4-hydroxyphenyl)methyl group, (5-methyl-2-oxo-1,3-dioxol-4-yl)methyl group, (5-methyl-2-oxo-1,3-dioxol-4-yl)methoxycarbonyl group, tetrahydrofuranyl group, pyrrolidylmethyl group and the like.

The term “pharmaceutical composition” includes a mixture comprising one or more active ingredients and one or more pharmaceutically acceptable carriers, for example, oral preparations such as tablet, capsule, granule, powder, troche, syrup, emulsion suspension and the like or parenteral preparations such as external preparation, suppository, injection, eye drop, a preparation for transnasal administration and a preparation for lung administration and the like.

Pharmaceutical compositions of the present invention can be prepared by mixing suitably the compounds of Formula [I] and the like or pharmaceutically acceptable salts thereof with at least one pharmaceutically acceptable carrier and the like, by conventional methods in the art of medicinal preparations. Content rate of the compounds of Formula [I] and the like or pharmaceutically acceptable salts thereof in the pharmaceutical composition includes for example, 0.1 to 100%, preferably 0.1 to 70% by weight in the composition while it varies depending on dosage forms, dosage amounts and the like.

The term “pharmaceutically acceptable carriers” includes all sorts of organic or inorganic carriers which are commonly-used as a material for drug formulations, such as excipient, disintegrant, binder, fluidizer, lubricant and the like for solid preparations and solvent, solubilizing agent, suspending agent, tonicity agent, buffering agent, soothing agent and the like for liquid preparations. Such preparations may employ further additives such as preservative, antioxidant, colorant, sweetening agent and the like as necessary.

The term “excipient” includes for example, lactose, white soft sugar, D-mannitol, D-sorbitol, cornstarch, dextrin, microcrystalline cellulose, crystalline cellulose, carmellose, carmellose calcium, sodium carboxymethylstarch, low substituted hydroxypropylcellulose, gum arabic and the like.

The term “disintegrant” includes for example, carmellose, carmellose calcium, carmellose sodium, sodium carboxymethylstarch, croscarmellose sodium, crospovidone, low substituted hydroxypropylcellulose, hydroxypropylmethylcellulose, crystalline cellulose and the like.

The term “binder” includes for example, hydroxypropylcellulose, hydroxypropylmethylcellulose, povidone, crystalline cellulose, white soft sugar, dextrin, starch, gelatin, carmellose sodium, gum arabic and the like.

The term “fluidizer” includes for example, light anhydrous silicic acid, magnesium stearate and the like.

The term “lubricant” includes for example, magnesium stearate, calcium stearate, talc and the like.

The term “solvent” includes for example, purified water, ethanol, propyleneglycol, macrogol, sesame oil, corn oil, olive oil and the like.

The term “solubilizing agent” includes for example, propyleneglycol, D-mannitol, benzyl benzoate, ethanol, triethanolamine, sodium carbonate, sodium citrate and the like.

The term “suspending agent” includes for example, benzalkonium chloride, carmellose, hydroxypropylcellulose, propyleneglycol, povidone, methylcellulose, glyceryl monostearate and the like.

The term “tonicity agent” includes for example, glucose, D-sorbitol, sodium chloride, D-mannitol and the like.

The term “buffering agent” includes for example, disodium hydrogen phosphate, sodium acetate, sodium carbonate, sodium citrate and the like.

The term “soothing agent” includes for example, benzyl alcohol and the like.

The term “preservative” includes for example, methyl parahydroxybenzoate, ethyl parahydroxybenzoate, propyl parahydroxybenzoate, chlorobutanol, benzyl alcohol, sodium dehydroacetate, sorbic acid and the like.

The term “antioxidant” includes for example, sodium sulfite, ascorbic acid and the like.

The term “colorant” includes for example, food dye such as Food Red No. 2 and No. 3, Food Yellow No. 4 and No. 5 and the like, β-carotene and the like.

The term “sweetening agent” includes for example saccharin sodium, dipotassium glycyrrhizate, aspartame and the like.

The pharmaceutical compositions of the present invention can be administered to human as well as mammals other than human such as mice, rat, hamster, guinea pig, rabbit, cat, dog, pig, cattle, horse, sheep, monkey and the like orally or parenterally such as locally, rectally and intravenously. While the dosage amount may vary depending on subject, disease, symptom, dosage form, route of administration and the like, for example when it is administered orally to an adult patient with autoimmune disease, allergic disease, dry eye, fibrosis, or metabolic disease (body weight: about 60 kg) the dosage amount of the present invention compound of an active ingredient ranges generally from about 1 mg to about 1 g per day, which can be administered once to several times.

The compounds of Formula [I] and the like or pharmaceutically acceptable salts thereof can inhibit RORγ, thereby they can be used as an active ingredient for treating or preventing a disease such as autoimmune disease such as rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica, and type I diabetes; allergic disease such as asthma; dry eye; fibrosis; and metabolic disease such as diabetes.

“Inhibit RORγ” means that a function of RORγ is inhibited to make the activity thereof disappear or reduced. It includes, for example, the function of RORγ is inhibited according to Biological assay 1 described hereafter.

The preferred aspect of “Inhibit RORγ” includes “Inhibit human RORγ”.

The compounds of Formula [I] and the like or pharmaceutically acceptable salts thereof can be used in combination with other one or more medicament (hereinafter called additional medicament(s)) according to methods commonly used in the art of medicine, which is hereinafter called combination use.

The timing of administration of the compounds of Formula [I] and the like or pharmaceutically acceptable salts thereof and additional medicament(s) is not limited and they may be administered to a subject in a form of combination drug or may be administered simultaneously or at regular intervals. In addition, the compounds of Formula [I] and the like or pharmaceutically acceptable salts thereof may be used as a kit comprising the pharmaceutical composition of the present invention and additional medicament(s).

The dosage amount of the additional medicament(s) may follow one employed in clinical practice, and may be determined appropriately depending on subject, disease, symptom, dosage form, route of administration, timing of administration, combination and the like. The mode of additional medicament(s) is not limited as long as the present invention compounds or salts thereof and the additional medicament(s) are combined.

The additional medicament(s) include for example,

(1) a medicament for treating or preventing autoimmune disease such as rheumatoid arthritis, psoriasis, inflammatory bowel disease such as Crohn's disease and ulcerative colitis, multiple sclerosis, systemic lupus erythematosus, ankylosing spondylitis, uveitis, polymyalgia rheumatica, and type I diabetes;

(2) a medicament for treating or preventing allergic disease such as asthma;

(3) a medicament for treating or preventing metabolic disease such as diabetes;

(4) a medicament for treating or preventing dry eye; and

(5) a medicament for treating or preventing fibrosis.

One to three medicament selected from the above (1) to (5) may be employed in combination with the compounds of Formula [I] and the like or pharmaceutically acceptable salts thereof.

The medicament for treating or preventing autoimmune disease includes, for example, methotrexate to treat or prevent rheumatoid arthritis, and ciclosporin A and methotrexate to treat or prevent psoriasis.

Next, some examples of processes for preparing the compound of the present invention described in the above [01] to [31] are shown as follows. However, the processes for preparing the present invention compound should not be limited thereto.

It is possible to modify the processes to carry out the preparation more effectively, for example, introducing a protecting group into a functional group followed by deprotecting it in a subsequent step; using a precursor having a functional group in a step followed by converting it to the desired functional group in a subsequent step; exchanging the order of preparation methods or steps thereof, even though not mentioned in these examples.

The workup after the reaction in each step can be carried out by a commonly-used method, wherein the isolation and purification may be carried out by a conventional method selected from crystallization, recrystallization, distillation, separating, silicagel chromatography, preparative HPLC and the like, or a combination thereof, as appropriate.

A racemic form of the compound can be obtained by using an achiral compound as a material, ligand, or reagent, or by mixing of enantiomers.

The following abbreviations are used in the preparation methods and Examples herein:
tert-butyldimethylsilyl group (TBDMS)
tert-butyldiphenylsilyl group (TBDPS)
tert-butoxycarbonyl group (Boc)
benzyl group (Bn)
phenyl group (Ph)
n-butyl group (nBu)
tert-butyl group (tBu)
isopropyl group (iPr)
ethyl group (Et)
methyl group (Me)
N,N-diisopropylethylamine (DIPEA)
lithium diisopropylamide (LDA)
diisobutylaluminium hydride (DIBAL)
1-ethyl-3-(3-dimethylaminopropyl)carbodiimide hydrochloride (WSC.HCl)
1-hydroxy-1H-benzotriazole monohydrate (HOBt.H₂O)
O-(7-azabenzotriazol-1-yl)-N,N,N′,N′-tetramethyluronium hexafluorophosphate (HATU)
tetrabutylammonium fluoride (TBAF)
1,8-diazabicyclo[5.4.0]undec-7-ene (DBU)
trifluoroacetic acid (TFA)
trifluoroacetic anhydride (TFAA)
Dess-Martin reagent (DMP)
lithium hexamethyldisilazide (LHMDS)
4-dimethylaminopyridine (DMAP)
2,2,6,6-tetramethyl-1-piperidinyloxy radical (TEMPO)
2-azaadamantane-N-oxyl (AZADO)
(diethylamino)sulfur trifluoride (DAST)
bis(2-methoxyethyl)aminosulfur trifluoride (Deoxo-Fluor (registered trademark))
1,1′-bis(diphenylphosphino)ferrocene (dppf)
dimethyl sulfoxide (DMSO)
N,N-dimethylformamide (DMF)
tetrahydrofuran (THF)
N,N-dimethylacetamide (DMA)
hexamethylphosphoric triamide (HMPA)
enantiomer excess (ee)
room temperature (rt)
high-performance liquid chromatography (HPLC)

In the following schemes,

“X” is a leaving group such as halogen atom, trifluoromethanesulfonyloxy group and the like, preferably bromo group, and iodine group;

“P^{N1}” is a protecting group for amine, and includes for example, tert-butoxycarbonyl group, benzyloxycarbonyl group and the like, preferably tert-butoxycarbonyl group.

“P^{O}” and “P^{O1}” is a protecting group for hydroxyl group, and includes for example, tert-butyldimethylsilyl group, tert-butyldiphenylsilyl group, acetyl group, benzyl group and the like, preferably tert-butyldimethylsilyl group, tert-butyldiphenylsilyl group, and benzyl group.

“P^{C}” is a protecting group for carboxyl group, and includes for example, methyl group, ethyl group, tert-butyl group, benzyl group and the like, preferably methyl group, tert-butyl group, and benzyl group.

“AUX-H” is a chiral auxiliary reagent, and includes for example (R)-4-benzyl-2-oxazolidinone, (S)-4-benzyl-2-oxazolidinone, (R)-4-isopropyl-2-oxazolidinone, (S)-4-isopropyl-2-oxazolidinone, (4S,5R)-4-methyl-5-phenyl-2-oxazolidinone, (4R,5S)-4-methyl-5-phenyl-2-oxazolidinone and the like, preferably (R)-4-benzyl-2-oxazolidinone, (S)-4-benzyl-2-oxazolidinone.

“AUX” is a chiral auxiliary group.

“Q” is a group comprising boron, magnesium, zinc, tin or the like, and includes for example, boronic acid, dialkoxyboron, halogeno magnesium, halogeno zinc, and trialkyltin.

Unless otherwise instructed, each symbol is as defined in the above [01].

### Preparation Method 1

(In the formula [I], R^{c} is hydrogen atom.)

Each step in Preparation method 1 is explained as follows.

(Step 1)

Compound [Y-11] can be obtained from Compound [Y-10] in a solvent under the acidic condition of ester hydrolysis reaction.

Examples of the acid for the reaction include hydrochloric acid, hydrobromic acid, sulfuric acid, trifluoroacetic acid. The preferred acid for the reaction includes hydrochloric acid, hydrobromic acid, trifluoroacetic acid.

Examples of the solvent for the reaction include hydrocarbon solvent such as benzene, toluene, xylene, and hexane; halogenated solvent such as dichloromethane, chloroform, carbon tetrachloride, and 1,2-dichloroethane; ethers solvent such as diethyl ether, tetrahydrofuran, dioxane, 1,2-dimethoxyethane, and diglyme; esters solvent such as ethyl acetate, methyl acetate, and butyl acetate; polar solvent such as acetic acid and water. Preferred examples of the solvent for the reaction include dichloromethane, chloroform, toluene, acetic acid, and water.

The reaction temperature usually ranges about 0° C. to 120° C., preferably from room temperature to 100° C.

The reaction time usually ranges about 10 minutes to 3 days, preferably about 30 minutes to 1 day.

(Step 2)

Compound [Y-13] can be obtained by reacting Compound [Y-11] with [Y-12] under a commonly-used amide bond formation reaction. For example, Compound [Y-13] can be obtained by reacting Compound [Y-11] with [Y-12] in the presence of a condensation reagent in a solvent.

Examples of the solvent for the reaction include hydrocarbon solvent such as benzene and toluene; halogenated solvent such as dichloromethane, chloroform, carbon tetrachloride, and 1,2-dichloroethane; ethers solvent such as tetrahydrofuran, dioxane, and 1,2-dimethoxyethane; polar solvent such as N,N-dimethylformamide, dimethyl sulfoxide, acetonitrile, and acetone; which may be used alone or as a mixture of two or more. Preferred examples of the solvent for the reaction include dichloromethane, chloroform, and N,N-dimethylformamide.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), carbonyldiimidazole (CDI), and O-(7-azabenzotriazol-1-yl)-N,N,N′,N′,-tetramethyluronium hexafluorophosphate (HATU). As necessary, 1-hydroxy-1H-benzotriazole monohydrate (HOBt.H₂O), 4-dimethylaminopyridine (DMAP), N,N-diisopropylethylamine, and the like may be added. Preferred examples of the condensation reagent include a mixture of water-soluble carbodiimide (WSC.HCl: 1-ethyl-3-(3-dimethylaminopropyl)carbodiimide hydrochloride) and 1-hydroxy-1H-benzotriazole monohydrate (HOBt.H₂O) and a mixture of O-(7-azabenzotriazol-1-yl)-N,N,N′,N′,-tetramethyluronium hexafluorophosphate (HATU) and N,N-diisopropylethylamine.

The reaction temperature usually ranges about 0° C. to 120° C., preferably from room temperature to 100° C.

The reaction time usually ranges about 30 minutes to 3 days, preferably about 1 hr to 1 day.

Alternatively, in the above amidation reaction, Compound [Y-13] can be prepared by the reaction of an acid halide or mixed acid anhydride of Compound [Y-11] with Compound [Y-12].

The acid halide of Compound [Y-11] can be derived by the reaction of a carboxylic acid of Compound [Y-11] with thionyl chloride, oxalyl chloride etc. wherein a catalytic amount of N,N-dimethylformamide may be added.

The mixed acid anhydride of Compound [Y-11] can be derived by the reaction of a carboxylic acid of Compound [Y-11] with ethyl chlorocarbonate etc.

(Step 3)

Compound [Y-14] can be obtained by removal of the protecting group P^{O} from Compound [Y-13] in a solvent.

When the protecting group P^{O} is benzyl group, the protecting group may be removed by a reaction in the presence of Lewis acid.

Examples of Lewis acid for the reaction include boron tribromide, boron trichloride, and trimethylsilyl iodide.

Preferred examples of Lewis acid for the reaction include boron tribromide.

Examples of the solvent for the reaction include hydrocarbon solvent such as benzene, toluene, xylene, and hexane; halogenated solvent such as dichloromethane, chloroform, carbon tetrachloride, and 1,2-dichloroethane; which may be used alone or as a mixture of two or more. Preferred examples of the solvent for the reaction include dichloromethane and chloroform.

The reaction temperature usually ranges about −78° C. to 50° C., preferably about −78° C. to room temperature.

The reaction time usually ranges about 30 minutes to 3 days, preferably about 1 hr to 1 day.

(Step 4)

Compound [I] (R^{c}═H) can be obtained by the reaction of Compound [Y-14] in the presence of an oxidant in a solvent.

Examples of the oxidant for the reaction include 2,2,6,6-tetramethyl-1-piperidinyloxy radical (TEMPO), sodium hypochlorite, sodium chlorite, potassium permanganate, aqueous hydrogen peroxide, pyridinium dichromate, and chromium oxide. Preferred examples of the oxidant for the reaction include a mixture of 2,2,6,6-tetramethyl-1-piperidinyloxy radical (TEMPO), sodium hypochlorite, and sodium chlorite.

Examples of the solvent for the reaction include halogenated solvent such as dichloromethane, chloroform, carbon tetrachloride, and 1,2-dichloroethane; ethers solvent such as diethyl ether, tetrahydrofuran, dioxane, 1,2-dimethoxyethane, and diglyme; polar solvent such as acetonitrile, acetone, and water; which may be used alone or as a mixture of two or more. Preferred examples of the solvent for the reaction include acetonitrile and water.

The reaction temperature usually ranges about −10° C. to 150° C., preferably about 0° C. to 80° C.

The reaction time usually ranges about 30 minutes to 5 days, preferably about 1 hr to 1 day.

(Step 5) Salt Formation Reaction

According to conventional methods, each salt can be obtained by reacting a compound represented by Formula [I] wherein R^{c} is hydrogen atom with an inorganic base. For example, carboxylic acid [I] wherein R^{c} is hydrogen atom is reacted with aqueous sodium hydroxide in a alcohol solvent at room temperature to give the sodium salt.

### Preparation Method 2

The compound wherein

is

and

is

specifically, a compound of the following [II-A]:

wherein n^{a} is 1 or 2, and R^{c} is hydrogen atom.

### Preparation Method 2A-1

(Step 1)

Compound [Y-17] can be obtained by reacting Compound [Y-15] with AUX-H[Y-16] in the presence of a condensation reagent in a solvent.

Examples of the solvent for the reaction include hydrocarbon solvent such as benzene, toluene, xylene, and hexane; halogenated solvent such as dichloromethane and chloroform; ethers solvent such as tetrahydrofuran, dioxane, and 1,2-dimethoxyethane; polar solvent such as N,N-dimethylformamide, dimethyl sulfoxide, acetonitrile, and acetone; which may be used alone or as a mixture of two or more. Preferred examples of the solvent for the reaction include N,N-dimethylformamide and acetonitrile.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), and carbonyldiimidazole (CDI). As necessary, 1-hydroxy-1H-benzotriazole monohydrate (HOBt.H₂O), 4-dimethylaminopyridine (DMAP), and the like may be added. Preferred examples of the condensation reagent include a mixture of water-soluble carbodiimide (WSC.HCl: 1-ethyl-3-(3-dimethylaminopropyl)carbodiimide hydrochloride) and 4-dimethylaminopyridine (DMAP).

The reaction temperature usually ranges about room temperature to 120° C., preferably from room temperature to 80° C.

The reaction time usually ranges about 30 minutes to 3 days, preferably about 1 hr to 1 day.

### Example of AUX-H

(Step 2)

Compound [Y-19] can be obtained by reacting Compound [Y-17] with [Y-18] in the presence of a base in a solvent.

Examples of the solvent for the reaction include hydrocarbon solvent such as benzene, toluene, xylene, and hexane; ethers solvent such as diethyl ether, tetrahydrofuran, dioxane, 1,2-dimethoxyethane, and diglyme; which may be used alone or as a mixture of two or more.

Preferred examples of the solvent for the reaction include tetrahydrofuran.

Examples of the base for the reaction include sodium hexamethyldisilazide, lithium hexamethyldisilazide, and lithium diisopropylamide (LDA). Preferred examples of the base for the reaction include sodium hexamethyldisilazide.

The reaction temperature usually ranges about −78° C. to 50° C., preferably about −78° C. to room temperature.

The reaction time usually ranges about 30 minutes to 3 days, preferably about 30 minutes to 1 day.

(Step 3)

Compound [Y-20] can be obtained by hydrolysis reaction of Compound [Y-19] in a solvent.

The hydrolysis reaction may be performed under the commonly-used condition, for example, under the alkaline condition.

Examples of the base for the reaction include an aqueous solution of alkali metal hydroxide such as lithium hydroxide, sodium hydroxide, and potassium hydroxide; inorganic peroxide such as lithium peroxide, potassium peroxide, and sodium peroxide. Preferred examples of the base for the reaction include lithium peroxide. Examples of the solvent for the reaction include hydrocarbon solvent such as benzene, toluene, xylene, and hexane; alcohols solvent such as methanol, ethanol, isopropyl alcohol, and tert-butanol; halogenated solvent such as dichloromethane, chloroform, carbon tetrachloride, and 1,2-dichloroethane; ethers solvent such as diethyl ether, tetrahydrofuran, dioxane, 1,2-dimethoxyethane, and diglyme; polar solvent such as N,N-dimethylformamide, dimethyl sulfoxide, acetonitrile, acetone, and water; which may be used alone or as a mixture of two or more. Preferred examples of the solvent for the reaction include tetrahydrofuran and water.

The reaction temperature usually ranges about −30° C. to 80° C., preferably about 0° C. to room temperature.

The reaction time usually ranges about 30 minutes to 3 days, preferably about 30 minutes to 1 day.

(Purification Process)

An optical purity of Compound [Y-20] can be increased by a method such as the preferential crystallization and the diastereomer method in a solvent.

Preferred examples of amine for the crystallization include (R)-1-phenyl-ethylamine.

Examples of the solvent for the crystallization include esters solvent such as isopropyl acetate and n-butyl acetate; ethers solvent such as methyl-tert-butylether; ketone solvent such as methyl isobutyl ketone; which may be used alone or as a mixture of two or more. Preferred examples of the solvent for the reaction include methyl-tert-butylether.

Then, demineralization is performed with by using hydrochloric acid, potassium hydrogen sulfate etc.

An example of the steps from the Step 1 to the above purification process is following.

(R)-4-benzyl-2-oxazolidinone is used as AUX-H [Y-16], and (R)-1-phenyl-ethylamine is used as the amine in the purification process.

A case where an enantiomer is synthesized, (S)-4-benzyl-2-oxazolidinone may used instead of (R)-4-benzyl-2-oxazolidinone.

### Example of [Y-20]

(Step 4)

Compound [Y-21] can be obtained by reacting Compound [Y-20] with N,O-dimethylhydroxylamine or a hydrochloride salt thereof in the presence of a condensation reagent in a solvent.

Examples of the solvent for the reaction include hydrocarbon solvent such as benzene, toluene, xylene, and hexane; halogenated solvent such as dichloromethane and chloroform; ethers solvent such as tetrahydrofuran, dioxane, and 1,2-dimethoxyethane; polar solvent such as N,N-dimethylformamide, dimethyl sulfoxide, acetonitrile, and acetone; which may be used alone or as a mixture of two or more. Preferred examples of the solvent for the reaction include N,N-dimethylformamide, and acetonitrile.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), and carbonyldiimidazole (CDI). As necessary, 1-hydroxy-1H-benzotriazole monohydrate (HOBt.H₂O), 4-dimethylaminopyridine (DMAP), and the like may be added. Preferred examples of the condensation reagent include a mixture of water-soluble carbodiimide (WSC.HCl: 1-ethyl-3-(3-dimethylaminopropyl)carbodiimide hydrochloride) and 1-hydroxy-1H-benzotriazole monohydrate (HOBt.H₂O).

The reaction temperature usually ranges about room temperature to 120° C., preferably room temperature to 80° C.

The reaction time usually ranges about 30 minutes to 3 days, preferably about 1 hr to 1 day.

(Step 5)

Compound [Y-22] can be obtained by reacting Compound [Y-21] in the presence of a reducing agent in a solvent.

Examples of the solvent for the reaction include hydrocarbon solvent such as benzene, toluene, xylene, and hexane; halogenated solvent such as dichloromethane, chloroform, carbon tetrachloride, and 1,2-dichloroethane; ethers solvent such as diethyl ether, tetrahydrofuran, dioxane, 1,2-dimethoxyethane, and diglyme; which may be used alone or as a mixture of two or more. Preferred examples of the solvent for the reaction include toluene, dichloromethane, chloroform, hexane, and tetrahydrofuran.

Examples of the reducing agent for the reaction include diisobutylaluminium hydride and lithium aluminium hydride. Preferred examples of the reducing agent include diisobutylaluminium hydride.

The reaction temperature usually ranges about −78° C. to room temperature, preferably about −78° C. to 0° C.

The reaction time usually ranges about 30 minutes to 3 days, preferably about 1 hr to 1 day.

(Step 6)

Compound [Y-23] can be obtained by reacting Compound [Y-22] with hydroxylamine or a hydrochloride salt thereof in a solvent.

Examples of the solvent for the reaction include hydrocarbon solvent such as benzene, toluene, xylene, and hexane; ethers solvent such as diethyl ether, tetrahydrofuran, dioxane, 1,2-dimethoxyethane, and diglyme; polar solvent such as methanol, ethanol, acetonitrile, and water; which may be used alone or as a mixture of two or more. Preferred examples of the solvent for the reaction include toluene, ethanol, tetrahydrofuran, and water.

When the hydrochloride salt of hydroxylamine is used, examples of the base for the reaction include pyridine, triethylamine, sodium hydroxide, sodium acetate, potassium carbonate, sodium carbonate, and sodium hydrogen carbonate. Preferred examples of the base for the reaction include sodium hydroxide.

The reaction temperature usually ranges about −10° C. to 130° C., preferably about 0° C. to room temperature.

The reaction time usually ranges about 30 minutes to 3 days, preferably about 1 hr to 1 day.

(Step 7)

Compound [Y-24] can be obtained by reacting Compound [Y-23] in the presence of a chlorinating agent in a solvent.

Examples of the solvent for the reaction include halogenated solvent such as dichloromethane, chloroform, carbon tetrachloride, and 1,2-dichloroethane; polar solvent such as N,N-dimethylformamide and acetic acid; which may be used alone or as a mixture of two or more. Preferred examples of the solvent for the reaction include N,N-dimethylformamide.

Examples of the halogenating agent for the reaction include chlorine and N-chlorosuccinimide. Preferred examples of the for the reaction include chlorinating agent include N-chlorosuccinimide.

The reaction temperature usually ranges about −10° C. to 100° C., preferably about 0° C. to 60° C.

The reaction time usually ranges about 30 minutes to 3 days, preferably about 1 hr to 1 day.

### Preparation Method 2A-2

wherein n^{a} is 1.

(Step 1)

Compound [Y-26] can be obtained by reacting Compound [Y-24] with propargyl alcohol [Y-25] in the presence of a base in a solvent.

Examples of the solvent for the reaction include hydrocarbon solvent such as benzene, toluene, xylene, and hexane; ethers solvent such as diethyl ether, tetrahydrofuran, dioxane, 1,2-dimethoxyethane, and diglyme; water; which may be used alone or as a mixture of two or more. Preferred examples of the solvent for the reaction include toluene, and water.

Examples of the base for the reaction include triethylamine, sodium hydroxide, potassium carbonate, sodium carbonate, and sodium hydrogen carbonate. Preferred examples of the base for the reaction include potassium carbonate.

The reaction temperature usually ranges about 0° C. to 150° C., preferably from room temperature to 100° C.

The reaction time usually ranges about 30 minutes to 3 days, preferably about 1 hr to 1 day.

(Step 2)

Compound [Y-27] can be obtained by reacting Compound [Y-26] in the presence of a halogenating agent in a solvent.

Examples of the solvent for the reaction include halogenated solvent such as dichloromethane, chloroform, carbon tetrachloride, and 1,2-dichloroethane; polar solvent such as N,N-dimethylformamide and acetic acid; which may be used alone or as a mixture of two or more. Preferred examples of the solvent for the reaction include N,N-dimethylformamide, and acetonitrile.

Examples of the halogenating agent for the reaction include bromine, iodine, iodine monochloride, N-bromosuccinimide, N-iodosuccinimide, and 1,3-dibromo-5,5-dimethylhydantoin. As necessary, cerium(IV) diammonium nitrate, trifluoroacetic acid, and the like may be added. Preferred examples of the halogenating agent for the reaction include a mixture of N-iodosuccinimide and cerium(IV) diammonium nitrate.

The reaction temperature usually ranges about −10° C. to 100° C., preferably about 0° C. to 50° C.

The reaction time usually ranges about 1 hr to 3 days, preferably about 3 hrs to 1 day.

(Step 3)

Compound [Y-28] can be obtained by the reaction of Compound [Y-27] in the presence of an oxidant in a solvent.

Examples of the oxidant for the reaction include 2,2,6,6-tetramethyl-1-piperidinyloxy radical (TEMPO), manganese dioxide, Dess-Martin reagent (DMP), and sulfur trioxide-pyridine complex. Preferred examples of the oxidant for the reaction include Dess-Martin reagent (DMP).

Examples of the solvent for the reaction include hydrocarbon solvent such as benzene, toluene, xylene, and hexane; halogenated solvent such as dichloromethane, chloroform, carbon tetrachloride, and 1,2-dichloroethane; ethers solvent such as diethyl ether, tetrahydrofuran, dioxane, 1,2-dimethoxyethane, and diglyme; polar solvent such as acetonitrile, acetone, and water; which may be used alone or as a mixture of two or more. Preferred examples of the solvent for the reaction include dichloromethane and chloroform.

The reaction temperature usually ranges about −78° C. to 80° C., preferably about 0° C. to room temperature.

The reaction time usually ranges about 10 minutes to 1 day, preferably about 10 minutes to 5 hrs.

(Step 4)

Compound [Y-29] can be obtained by reacting Compound [Y-28] with hydroxylamine or a hydrochloride salt thereof in a solvent.

The solvent, the reactant, the reaction temperature, the reaction time, for the reaction, are similar to those of Step 6 of Preparation method 2A-1.

(Step 5)

Compound [Y-30] can be obtained by reacting Compound [Y-29] in the presence of a chlorinating agent in a solvent.

The solvent, the reactant, the reaction temperature, and the reaction time, for the reaction are similar to those of Step 7 of Preparation method 2A-1.

(Step 6)

Compound [Y-32] can be obtained by reacting Compound [Y-30] with [Y-31] in the presence of a base in a solvent.

The solvent, the base, the reaction temperature, the reaction time, for the reaction, are similar to those of Step 1 of Preparation method 2A-2.

(Step 7)

Compound [Y-34] can be obtained by reacting Compound [Y-32] with Compound [Y-33] in the presence of a metal catalyst in a solvent.

As the Compound [Y-33], alkylboronic acid, alkylboronic acid ester, alkylzinc reagent, alkylmagnesium reagent, and the like may be used, preferably alkylboronic acid ester may be used.

Examples of the solvent for the reaction include hydrocarbon solvent such as benzene, toluene, xylene, and hexane; ethers solvent such as diethyl ether, tetrahydrofuran, dioxane, 1,2-dimethoxyethane, and diglyme; polar solvent such as N,N-dimethylformamide, dimethyl sulfoxide, acetonitrile, acetone, and water; which may be used alone or as a mixture of two or more. Preferred examples of the solvent for the reaction include N,N-dimethylformamide.

Examples of the metal catalyst for the reaction include a palladium catalyst such as bis(triphenylphosphine)palladium(II) dichloride and 1,1′-bis(diphenylphosphino)ferrocene-palladium(II) dichloride; a nickel catalyst such as [1,2-bis(diphenylphosphino)ethane]nickel(II) dichloride and nickel(II) acetylacetonate; and an iron catalyst such as iron(III) chloride. Preferred examples of the metal catalyst include bis(triphenylphosphine)palladium(II) dichloride.

As necessary, a base or an inorganic salt may be added. Examples of the base or the inorganic salt for the reaction include alkali metal phosphate such as tripotassium phosphate; alkali metal carbonate such as sodium carbonate and potassium carbonate; alkali metal acetate such as sodium acetate; and fluoride salt such as cesium fluoride, preferred examples include preferably tripotassium phosphate.

The reaction temperature usually ranges about −10° C. to 150° C., preferably about 0° C. to 120° C.

The reaction time usually ranges about 30 minutes to 3 days, preferably about 1 hr to 1 day.

(Step 6A)

Example of reaction process of the compound wherein R^{a1} is halogen atom:

wherein R^{a10} together with the —CF₂— forms R^{a1}.

(Step 6A-1)

Compound [Y-36] can be obtained by reacting Compound [Y-30] with [Y-35] in the presence of a base in a solvent.

The solvent, the base, the reaction temperature, the reaction time, for the reaction, are similar to those of Step 6 of Preparation method 2A-2.

(Step 6A-2)

Compound [Y-37] can be obtained by the reaction of Compound [Y-36] in the presence of an oxidant in a solvent.

Examples of the oxidant for the reaction include 2-azaadamantane-N-oxyl (AZADO), 2,2,6,6-tetramethyl-1-piperidinyloxy radical (TEMPO), manganese dioxide, Dess-Martin reagent, and sulfur trioxide-pyridine complex. Preferred examples of the oxidant for the reaction include Dess-Martin reagent.

Examples of the solvent for the reaction include hydrocarbon solvent such as benzene, toluene, xylene, and hexane; halogenated solvent such as dichloromethane, chloroform, carbon tetrachloride, and 1,2-dichloroethane; ethers solvent such as diethyl ether, tetrahydrofuran, dioxane, 1,2-dimethoxyethane, and diglyme; water; which may be used alone or as a mixture of two or more. Preferred examples of the solvent for the reaction include dichloromethane and chloroform.

The reaction temperature usually ranges about −78° C. to 80° C., preferably about 0° C. to room temperature.

The reaction time usually ranges about 10 minutes to 3 days, preferably about 30 minutes to 1 day.

(Step 6A-3)

Compound [Y-38] can be obtained by reacting Compound [Y-37] with a fluorinating agent.

Examples of the fluorinating agent for the reaction include (diethylamino)sulfur trifluoride (CAST), and bis(2-methoxyethyl)aminosulfur trifluoride (Deoxo-Fluor (registered trademark)). Preferred examples of the fluorinating agent for the reaction include bis(2-methoxyethyl)aminosulfur trifluoride (Deoxo-Fluor (registered trademark)).

The reaction temperature usually ranges about −10° C. to 120° C., preferably from room temperature to 100° C.

The reaction time usually ranges about 30 minutes to 3 days, preferably about 1 hr to 1 day.

### Preparation Method 2A-3

Preparation Method of Compound [Y-34] through Compound [Y-39]:

Compound [Y-34] can also be prepared by obtaining Compound [Y-39] from Compound [Y-27].

(Step 1)

Compound [Y-39] can be obtained by reacting Compound [Y-27] with Compound [Y-33] in the presence of a metal catalyst in a solvent.

The solvent, the reactant, the reaction temperature, and the reaction time, for the reaction, are similar to those of Step 7 of Preparation method 2A-2.

(Step 2 and the Following Steps)

Compound [Y-34] can be obtained from Compound [Y-39] by oxidation (Step 2), oximation (Step 3), chlorination (Step 4), and isoxazole cyclization (Step 5).

The solvent, the reactant, the reaction temperature, and the reaction time, for the reaction, are similar to those of Steps 3 to 6 of Preparation method 2A-2.

### Preparation Method 2B-1

(Step 1)

Compound [Y-41] can be obtained by reacting Compound [Y-40] with N,O-dimethylhydroxylamine or a hydrochloride salt thereof in the presence of a condensation reagent in a solvent.

The solvent, the reactant, the reaction temperature, and the reaction time, for the reaction, are similar to those of Step 4 of Preparation method 2A-1.

(Step 2)

Compound [Y-42] can be obtained by reacting Compound [Y-41] in the presence of a reducing agent in a solvent.

The solvent, the reactant, the reaction temperature, and the reaction time, for the reaction, are similar to those of Step 5 of Preparation method 2A-1.

(Step 3)

Compound [Y-43] can be obtained by reacting Compound [Y-42] in the presence of carbon tetrabromide and triphenylphosphine in a solvent.

Examples of the solvent for the reaction include hydrocarbon solvent such as benzene, toluene, xylene, and hexane; halogenated solvent such as dichloromethane, chloroform, carbon tetrachloride, and 1,2-dichloroethane; ethers solvent such as diethyl ether, tetrahydrofuran, dioxane, 1,2-dimethoxyethane, and diglyme; which may be used alone or as a mixture of two or more. Preferred examples of the solvent for the reaction include toluene and dichloromethane.

The reaction temperature usually ranges about −30° C. to 100° C., preferably about −10° C. to room temperature.

The reaction time usually ranges about 30 minutes to 3 days, preferably about 30 minutes to 1 day.

### Preparation Method 2B-2

(Step 1)

Compound [Y-44] can be obtained by reacting Compound [Y-43] with Compound [Y-21] in the presence of a base in a solvent.

Examples of the solvent for the reaction include hydrocarbon solvent such as benzene, toluene, xylene, and hexane; halogenated solvent such as dichloromethane, chloroform, carbon tetrachloride, and 1,2-dichloroethane; ethers solvent such as diethyl ether, tetrahydrofuran, dioxane, 1,2-dimethoxyethane, and diglyme; which may be used alone or as a mixture of two or more. Preferred examples of the solvent for the reaction include tetrahydrofuran, toluene, and hexane.

Examples of the base for the reaction include n-butyllithium, methyllithium, ethylmagnesium bromide, and lithium diisopropylamide (LDA). Preferred examples of the base for the reaction include n-butyllithium.

The reaction temperature usually ranges about −78° C. to 50° C., preferably about −78° C. to room temperature.

The reaction time usually ranges about 30 minutes to 3 days, preferably about 1 hr to 1 day.

(Step 2)

Compound [Y-45] can be obtained by reacting Compound [Y-44] with O-methylhydroxylamine or a hydrochloride salt thereof in a solvent.

Examples of the solvent for the reaction include alcohols solvent such as methanol, ethanol, and isopropyl alcohol; hydrocarbon solvent such as benzene, toluene, xylene, and hexane; halogenated solvent such as dichloromethane, chloroform, carbon tetrachloride, and 1,2-dichloroethane; ethers solvent such as diethyl ether, tetrahydrofuran, dioxane, 1,2-dimethoxyethane, and diglyme; polar solvent such as N,N-dimethylformamide, dimethyl sulfoxide, acetonitrile, and acetone; water, pyridine; which may be used alone or as a mixture of two or more. Preferred examples of the solvent for the reaction include ethanol.

When the hydrochloride salt of hydroxylamine is used, examples of the base for the reaction include pyridine, triethylamine, sodium hydroxide, sodium acetate, potassium carbonate, sodium carbonate, and sodium hydrogen carbonate. Preferred examples of the base for the reaction include sodium carbonate.

The reaction temperature usually ranges about −10° C. to 120° C., preferably room temperature to 100° C.

The reaction time usually ranges about 30 minutes to 3 days, preferably about 1 hr to 1 day.

(Step 3)

Compound [Y-46] can be obtained by the cyclization reaction of Compound [Y-45] in the presence of halogen or organohalide in a solvent.

Examples of the solvent for the reaction include hydrocarbon solvent such as benzene, toluene, xylene, and hexane; halogenated solvent such as dichloromethane, chloroform, carbon tetrachloride, and 1,2-dichloroethane; ethers solvent such as diethyl ether, tetrahydrofuran, dioxane, and 1,2-dimethoxyethane; polar solvent such as acetonitrile; which may be used alone or as a mixture of two or more. Preferred examples of the solvent for the reaction include dichloromethane. Examples of the halogen or the organohalide for the reaction include bromine, iodine, N-bromosuccinimide, N-iodosuccinimide, and iodine monochloride. Preferred examples of the halogen or the organohalide for the reaction include iodine, and iodine monochloride.

The reaction temperature usually ranges about −30° C. to 50° C., preferably about 0° C. to room temperature.

The reaction time usually ranges about 30 minutes to 3 days, preferably about 30 minutes to 1 day.

(Step 4)

Compound [Y-47] can be obtained by reacting Compound [Y-46] with Compound [Y-33] in the presence of a metal catalyst in a solvent.

The solvent, the reactant, the reaction temperature, and the reaction time, for the reaction, are similar to those of Step 7 of Preparation method 2A-2.

Preparation Method 2B-1 and Preparation method 2B-2 of a compound wherein Moiety U is isoxazole, thiazole, or furan correspond to the following.

### Preparation Method 2B-3

### Moiety U is Isoxazole:

Compound [Y-49] can be obtained from Compound [Y-48]. The solvent, the reactant, the reaction temperature, the reaction time, for the reaction, are similar to those of Preparation method 2B-1.

Then, [Y-51] can be prepared by obtaining Compound [Y-50] from Compound [Y-49]. The solvent, the reactant, the reaction temperature, the reaction time, for the reaction, are similar to those of Preparation method 2B-2.

### Preparation Method 2B-4

### Moiety U is Thiazole:

Compound [Y-53] can be obtained from Compound [Y-52]. The solvent, the reactant, the reaction temperature, the reaction time, for the reaction, are similar to those of Preparation method 2B-1.

Then, [Y-55] can be prepared by obtaining Compound [Y-54] from Compound [Y-53]. The solvent, the reactant, the reaction temperature, the reaction time, for the reaction, are similar to those of Preparation method 2B-2.

### Preparation Method 2B-5

### Moiety U is Furan:

Compound [Y-57] can be obtained from Compound [Y-56]. The solvent, the reactant, the reaction temperature, the reaction time, for the reaction, are similar to those of Preparation method 2B-1.

[Y-59] can be prepared by obtaining Compound [Y-58] from Compound [Y-57]. The solvent, the reactant, the reaction temperature, the reaction time, for the reaction, are similar to those of Preparation method 2B-2.

### Preparation Method 2C

wherein:
n^{a} is 1 or 2; and
R^{c} is hydrogen atom.

Compound [II-A] (R^{c}═H) can be obtained from Compound [Y-34] by deprotection (Step 1), amidation reaction (Step 2), deprotection (Step 3), and oxidation (Step 4).

The solvent, the reactant, the reaction temperature, the reaction time, for the reaction, are similar to those of Steps 1 to 4 of Preparation method 1.

Likewise, Compound [I-A] (R^{c}═H) can be obtained from Compound [Y-47] by deprotection (Step 1), amidation reaction (Step 2), deprotection (Step 3), and oxidation (Step 4). The solvent, the reactant, the reaction temperature, the reaction time, for the reaction, are similar to those of Steps 1 to 4 of Preparation method 1.

### Preparation Method 3

Compound wherein

is

and

is

specifically, a compound of the following formula:

wherein
n^{a} is 1 or 2; and
R^{c} is hydrogen atom.

### Preparation Method 3A

(Step 1)

Compound [Y-64] can be obtained by reacting Compound [Y-63] with hydroxylamine or a hydrochloride salt thereof in a solvent.

The solvent, the reactant, the reaction temperature, the reaction time, for the reaction, are similar to those of Step 6 of Preparation method 2A-1.

(Step 2)

Compound [Y-65] can be obtained by reacting Compound [Y-64] in the presence of a chlorinating agent in a solvent.

The solvent, the reactant, the reaction temperature, the reaction time, for the reaction, are similar to those of Step 7 of Preparation method 2A-1.

(Step 3)

Compound [Y-67] can be obtained by reacting Compound [Y-65] with [Y-66] in the presence of a base in a solvent.

The solvent, the reactant, the reaction temperature, the reaction time, for the reaction, are similar to those of Step 1 of Preparation method 2A-2.

(Step 4)

Compound [Y-68] can be obtained by removal of the protecting group P^{O} from Compound [Y-67] in a solvent.

When the protecting group P^{O} is tert-butyldimethylsilyl group, the protecting group may be removed by the reaction in the presence of tetrabutylammonium fluoride (TBAF).

Examples of the solvent for the reaction include alcohols solvent such as methanol, ethanol, isopropyl alcohol, and tert-butanol; esters solvent such as ethyl acetate, methyl acetate, and butyl acetate; hydrocarbon solvent such as benzene, toluene, xylene, and hexane; ethers solvent such as diethyl ether, tetrahydrofuran, dioxane, 1,2-dimethoxyethane, and diglyme; polar solvent such as N,N-dimethylformamide, dimethyl sulfoxide, acetonitrile, and acetone; acetic acid, water; which may be used alone or as a mixture of two or more. Preferred examples of the solvent for the reaction include tetrahydrofuran.

The reaction temperature usually ranges about −10° C. to 80° C., preferably about 0° C. to 50° C.

The reaction time usually ranges about 30 minutes to 5 days, preferably about 1 hr to 1 day.

(Step 5)

Compound [Y-69] can be obtained by reacting Compound [Y-68] in the presence of a halogenating agent in a solvent.

The solvent, the reactant, the reaction temperature, the reaction time, for the reaction, are similar to those of Step 2 of Preparation method 2A-2.

(Supplementary Step 1)

Compound [Y-66] can be obtained by reacting Compound [Y-22] with trimethylsilyldiazomethane in the presence of a base in a solvent.

Examples of the solvent for the reaction include hydrocarbon solvent such as benzene, toluene, xylene, and hexane; ethers solvent such as diethyl ether, tetrahydrofuran, dioxane, 1,2-dimethoxyethane, and diglyme. Preferred examples of the solvent for the reaction include tetrahydrofuran and hexane.

Examples of the base for the reaction include n-butyllithium and lithium diisopropylamide (LDA). Preferred examples of the base for the reaction include n-butyllithium.

The reaction temperature usually ranges about −78° C. to 80° C., preferably about −78° C. to 0° C.

The reaction time usually ranges about 30 minutes to 3 days, preferably about 1 hr to 1 day.

### Preparation Method 3B

wherein n^{a} is 1 or 2.

Compound [Y-74] can be obtained from Compound [Y-69] by oxidation (Step 1), oximation reaction (Step 2), chlorination (Step 3), cyclization (Step 4), substitution reaction (Step 5).

The solvent, the reactant, the reaction temperature, the reaction time, for the reaction, are similar to those of Steps 3 to 7 of Preparation method 2A-2.

### Preparation Method 3C

### Wherein

n^{a} is 1 or 2; and
R^{c} is hydrogen atom.

Compound [II-B] (R^{c}═H) can be obtained from Compound [Y-74] by deprotection (Step 1), amidation reaction (Step 2), deprotection (Step 3), oxidation (Step 4).

The solvent, the reaction temperature, and the reaction time, for the reaction, are similar to those of Steps 1 to 4 of Preparation method 1.

### Preparation Method 4

Compound wherein

is

and

is

specifically, a compound of the following formula:

wherein n^{a} is 1 or 2.

### Preparation Method 4A

(Step 1)

Compound [Y-79] can be obtained by reacting Compound [Y-78] with hydroxylamine or a hydrochloride salt thereof in a solvent.

The solvent, the reactant, the reaction temperature, the reaction time, for the reaction, are similar to those of Step 6 of Preparation method 2A-1.

(Step 2)

Compound [Y-80] can be obtained by reacting Compound [Y-79] in the presence of a chlorinating agent in a solvent.

The solvent, the reactant, the reaction temperature, the reaction time, for the reaction, are similar to those of Step 7 of Preparation method 2A-1.

### Preparation Method 4B

### Wherein n^{a} is 1.

(Step 1)

Compound [Y-81] can be obtained by the reaction of Compound [Y-39] in the presence of an oxidant in a solvent.

The solvent, the reactant, the reaction temperature, the reaction time, for the reaction, are similar to those of Step 3 of Preparation method 2A-2.

(Step 2)

Compound [Y-82] can be obtained by reacting Compound [Y-81] with trimethylsilyldiazomethane in the presence of a base in a solvent.

The solvent, the reactant, the reaction temperature, the reaction time, for the reaction, are similar to those of Supplementary step 1 of Preparation method 3A.

(Step 3)

Compound [Y-83] can be obtained by reacting Compound [Y-82] with [Y-80] in the presence of a base in a solvent.

The solvent, the reactant, the reaction temperature, the reaction time, for the reaction, are similar to those of Step 1 of Preparation method 2A-2.

### Preparation Method 4C

wherein
n^{a} is 1 or 2; and

### R^{c}═H.

Compound [II-C] (R^{c}═H) can be obtained from Compound [Y-83] by deprotection (Step 1), amidation reaction (Step 2), deprotection (Step 3), and oxidation (Step 4).

The solvent, the reaction temperature, and the reaction time, for the reaction, are similar to those of Steps 1 to 4 of Preparation method 1.

### Preparation Method 5

Compound wherein

is

and

is selected from the following heterocycle:

(Step 1)

Compound [Y-87] can be obtained by the reaction of Compound [Y-39] in the presence of an oxidant in a solvent.

The solvent, the reactant, the reaction temperature, the reaction time, for the reaction, are similar to those of Step 4 of Preparation method 1.

(Step 2A)

### Moiety U is Pyrazole:

Weinreb amide derived from carboxylic acid (Compound [Y-87]) is reacted with acetylene in the presence of base to form ynone. The resulting ynone is reacted with hydrazine to form a pyrazole ring.

(Step 2B)

### Moiety U is Thiazole:

carboxylic acid (Compound [Y-87]) is condensed with α-aminoketone hydrochloride salt followed by a reaction with Lawesson's reagent to form a thiazole ring.

(Step 2C)

### Moiety U is Oxazole:

Carboxylic acid (Compound [Y-87]) is condensed with α-aminoketone hydrochloride salt followed by a reaction with Burgess' reagent to form an oxazole ring.

(Step 2D)

### Moiety U is [1,3,4]Thiadiazole:

An acid chloride derived from carboxylic acid (Compound [Y-87]) is reacted with thiohydrazide to form a [1,3,4]thiadiazole ring.

(Step 2E)

### Moiety U is [1,2,4]Oxadiazole:

A nitrile derived from carboxylic acid (Compound [Y-87]) is reacted with hydroxylamine to form an amidoxime. The resulting amidoxime is reacted with an acid chloride to form an [1,2,4]oxadiazole ring.

(Step 3)

wherein

is

and

is selected from the following heterocycle:

Compound [I] (R^{c}═H) can be obtained from Compound [Y-10] (Compound [Y-87], [Y-88], [Y-89], [Y-90], [Y-91], and the like) by deprotection (Step 1), amidation reaction (Step 2), deprotection (Step 3), oxidation (Step 4).

The solvent, the reaction temperature, and the reaction time, for the reaction, are similar to those of Steps 1 to 4 of Preparation method 1.

Next, some examples of processes for preparing the compounds described in [101] to [146] are shown as follows. However, the processes for preparing the compounds of the present invention should not be limited thereto.

Unless otherwise instructed, it is possible to modify the processes to carry out the preparation more effectively, for example, introducing a protecting group into a functional group followed by deprotecting it in subsequent step; using a precursor having a substituent from which a functional group of a final product can be derived, followed by converting it to the desired functional group in a subsequent and appropriate stage; exchanging the order of preparation methods or steps thereof.

The workup after the reaction in each step can be carried out by a commonly-used method, wherein the isolation and purification may be carried out by a conventional method selected from crystallization, recrystallization, distillation, separating, silicagel chromatography, preparative HPLC and the like, or a combination thereof, as appropriate.

A racemic form of the compound can be obtained by using an achiral compound as a material, ligand, or reagent, or by mixing of enantiomers.

The following abbreviations are used in the preparation methods and Examples herein:
tert-butyldimethylsilyl group (TBDPS)
tert-butyldiphenylsilyl group (TBDPS)
tert-butoxycarbonyl group (Boc)
benzyloxycarbonyl group (Z)
benzyl group (Bn)
phenyl group (Ph)
n-butyl group (nBu)
tert-butyl group (tBu)
isopropyl group (iPr)
ethyl group (Et)
methyl group (Me)
N,N-diisopropylethylamine (DIPEA)
lithium diisopropylamide (LDA)
diisobutylaluminium hydride (DIBAL)
1-ethyl-3-(3-dimethylaminopropyl)carbodiimide hydrochloride (WSC.HCl)
1-hydroxy-1H-benzotriazole monohydrate (HOBt.H₂O)
O-(7-azabenzotriazol-1-yl)-N,N,N′,N′-tetramethyluronium hexafluorophosphate (HATU)
tetrabutylammonium fluoride (TBAF)
1,8-diazabicyclo[5.4.0]undec-7-ene (DBU)
trifluoroacetic acid (TFA)
trifluoroacetic anhydride (TFAA)
Dess-Martin reagent (DMP)
lithium hexamethyldisilazide (LHMDS)
4-dimethylaminopyridine (DMAP)
2,2,6,6-tetramethyl-1-piperidinyloxy radical (TEMPO)
2-azaadamantane-N-oxyl (AZADO)
(diethylamino)sulfur trifluoride (DAST)
bis(2-methoxyethyl)aminosulfur trifluoride (Deoxo-Fluor (registered trademark))
1,1′-bis(diphenylphosphino)ferrocene (dppf) dimethyl sulfoxide (DMSO)
N,N-dimethylformamide (DMF)
tetrahydrofuran (THF)
N,N-dimethylacetamide (DMA)
hexamethylphosphoric triamide (HMPA)
enantiomer excess (ee)
room temperature (rt)
high-performance liquid chromatography (HPLC)

In the following schemes,

“X” is a leaving group such as halogen atom, trifluoromethanesulfonyloxy group, preferably bromo group and iodo group.

“P^{C}” is a protecting group for carboxyl group, and includes, for example, methyl group, ethyl group, tert-butyl group, and benzyl group, preferably methyl group, tert-butyl group, and benzyl group.

“AUX-H” is a chiral auxiliary reagent, and includes for example, (R)-4-benzyl-2-oxazolidinone, (S)-4-benzyl-2-oxazolidinone, (R)-4-isopropyl-2-oxazolidinone, (S)-4-isopropyl-2-oxazolidinone, (4S,5R)-4-methyl-5-phenyl-2-oxazolidinone, and (4R,5S)-4-methyl-5-phenyl-2-oxazolidinone, preferably (R)-4-benzyl-2-oxazolidinone, and (S)-4-benzyl-2-oxazolidinone.

### Example of AUX-H

“AUX” is a chiral auxiliary group.

In Preparation method 5′ to Preparation method 10, “R^{w}” means:

(i) “R^{w}” described in [102],

(ii) “R^{w}” protected by a protecting group,

(iii) a functional group which may be replaced with “R^{w}” by an appropriate reaction.

“R^{c}” means:

(i) “R^{c}” described in the above [103] to [111],

(ii) “R^{c}” protected by a protecting group, or

(iii) a functional group which may be replaced with “R^{c}” by an appropriate reaction.

“R^{a}” means:

(i) “R^{a}” described in the above [101] to [111],

(ii) “R^{a}” protected by a protecting group, or

(iii) a functional group which may be replaced with “R^{a}” by an appropriate reaction.

“R^{au}” means:

(i) “R^{au}” described in the above [108],

(ii) “R^{au}” protected by a protecting group, or

(iii) a functional group which may be replaced with “R^{au}” by an appropriate reaction.

“R^{a11}” may be selected from the following, depending on kinds of functional groups or types of reactions:

(i) “C₁₋₁₂ alkyl group which may optionally be substituted with the same or different 1 to 5 substituents selected from Group A” described in the above [101] to [111],

(ii) “C₁₋₁₂ alkyl group which may optionally be substituted with the same or different 1 to 5 substituents selected from Group AU” described in the above [108],

(iii) a group of the above (i) and (ii) which are protected by a protecting group, or

(iv) a group of the above (i) and (ii) which may be replaced with “R^{a11}” by an appropriate reaction.

When “R^{c}” is “—(CH₂)₂—C(═O)—OH”, examples of “R^{c}” protected by a protecting group include “—(CH₂)₂—C(═O)—OCH₃” and the like.

when “R^{c}” is “—(CH₂)₂-tetrazole”, examples of the functional group which may be replaced with “R^{c}” by an appropriate reaction includes “—(CH₂)₂—CN”, “—(CH₂)₂—CONH₂” and “—(CH₂)₂—O-TBDPS”.

In Preparation method 10-1 (including the reduction reaction), examples of “R^{a11}” include C₁₋₁₂ alkyl group.

Unless otherwise indicated, each symbol is as defined in the above [101] to [111], and is determined depending on the general formulas.

For example, “cyclic moiety UU” is “cyclic moiety UU” described in the above [108].

An optically-active compound can be obtained by separating a racemic compound by high-performance liquid chromatograph with a chiral stationary phase column.

The condition is, for example, as follows:

Instrument: Recycle preparative HPLC LC908 type (Japan Analytical Industry)
Column: DAICEL CHIRALPAK AD 20 mmφ×250 mm
Column temperature: room temperature
Mobile phase: ethanol
Flow rate: 0.5 ml/min

### Detection: UV (254 nm).

In the formulas, the symbol “*” denotes an asymmetric carbon.

### Preparation Method 5′

(5′-1) Amidation Reaction

Compound wherein Y is “single bond” or “C₁₋₃ alkylene which may be substituted with hydroxyl group”:

The above Preparation method 5′-1 is performed as follows.

### (Amidation Reaction Step)

Compound [III-X-D1] can be obtained by reacting Compound [Z-X-D1-501] with [Z-X-D1-502] in the presence of a condensation reagent in a solvent under the condition of a commonly-used amide bond formation reaction.

Examples of the solvent for the reaction include hydrocarbon solvent such as benzene, toluene, xylene, and hexane; halogenated solvent such as dichloromethane, chloroform, carbon tetrachloride, and 1,2-dichloroethane; ethers solvent such as diethyl ether, tetrahydrofuran, dioxane, 1,2-dimethoxyethane, and diglyme; polar solvent such as N,N-dimethylformamide, dimethyl sulfoxide, acetonitrile, and acetone; which may be used alone or as a mixture of two or more. Preferred examples of the solvent for the reaction include methylene chloride, chloroform, and N,N-dimethylformamide.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), carbonyldiimidazole (CDI), and O-(7-azabenzotriazol-1-yl)-N,N,N′,N′,-tetramethyluronium hexafluorophosphate (HATU). As necessary, 1-hydroxy-1H-benzotriazole monohydrate (HOBt.H₂O), 4-dimethylaminopyridine (DMAP), N,N-diisopropylethylamine, and the like may be added. Preferred examples of the condensation reagent include a mixture of water-soluble carbodiimide (WSC.HCl: 1-ethyl-3-(3-dimethylaminopropyl)carbodiimide hydrochloride) and 1-hydroxy-1H-benzotriazole monohydrate (HOBt.H₂O), and a mixture of O-(7-azabenzotriazol-1-yl)-N,N,N′,N′,-tetramethyluronium hexafluorophosphate (HATU) and N,N-diisopropylethylamine.

The reaction temperature usually ranges about room temperature to 120° C., preferably from room temperature to 100° C.

The reaction time usually ranges about 30 minutes to 3 days, preferably about 1 hr to 1 day.

Alternatively, in the above amidation reaction, Compound [III-X-D1] can be prepared by the reaction of an acid halide or mixed acid anhydride of Compound [Z-X-D1-501] with Compound [Z-X-D1-502].

The acid halide of [Z-X-D1-501] can be derived by the reaction of an carboxylic acid of compound [Z-X-D1-501] with thionyl chloride, oxalyl chloride etc., wherein a catalytic amount of N,N-dimethylformamide may be added.

The mixed acid anhydride of compound [Z-X-D1-501] can be derived by the reaction of a carboxylic acid of compound [Z-X-D1-501] with ethyl chlorocarbonate etc.

When Y is “C₁₋₃ alkylene which is substituted with hydroxyl group” in the above amidation reaction, the hydroxyl group of the alkylene may be protected.

In a similar way, compound 5B (right column) can be prepared from compound 5A (left column), as listed in the following table(s).

TABLE 74

| | Compound 5A | Compound 5B |
|---|---|---|
| 5-1 | [Z-J-R-501] | [III-J-B] |
| 5-2 | [Z-X-C-501] | [III-X-C] |
| 5-3 | [Z-J-C-501] | [III-J-C] |
| 5-4 | [Z-X-D2-501] | [III-X-D2] |
| 5-5 | [Z-J-D2-501] | [III-J-D2] |
| 5-6 | [Z-X-D1-501] | [III-X-D1] |
| 5-7 | [Z-J-D1-501] | [III-J-D1] |

In the following Table 75,

is

TABLE 75

| | Compound 5A | Compound 5B |
|---|---|---|
| 5-8 | [Z-X-D11-501] | [III-X-D11] |
| 5-9 | [Z-X-D12-501] | [III-X-D12] |

### (5′-2) Sulfonamidation Reaction

Compound wherein Y is “single bond” or “C₁₋₃ alkylene which may be substituted with hydroxyl group”:

Compound [III-X-D1-YS] can be obtained by reacting Compound [Z-X-D1-501] with [Z-X-D1-YS-502] in the presence of a condensation reagent in a solvent under a condition of a commonly-used sulfonamide bond formation reaction.

In a similar way, compound 5D (right column) can be prepared from compound 5C (left column), as listed in the following table(s).

TABLE 76

| | Compound 5C | Compound 5D |
|---|---|---|
| 5-10 | [Z-J-B-501] | [III-J-B-YS] |
| 5-11 | [Z-X-C-501] | [III-X-C-YS] |
| 5-12 | [Z-J-C-501] | [III-J-C-YS] |
| 5-13 | [Z-X-D2-501] | [III-X-D2-YS] |
| 5-14 | [Z-J-D2-501] | [III-J-D2-YS] |
| 5-15 | [Z-X-D1-501] | [III-X-D1-YS] |
| 5-16 | [Z-J-D1-501] | [III-J-D1-YS] |

In the following table,

is

In a similar way, compound 5B (right column) can be prepared from compound 5A (left column), as listed in the following table(s).

TABLE 77

| | Compound 5A | Compound 5B |
|---|---|---|
| 5-17 | [Z-X-D11-501] | [III-X-YS-D11] |
| 5-18 | [Z-X-D12-501] | [III-X-YS-D12] |

### Preparation Method 6

Ring-forming reactions are explained below.

### Preparation Method 6F

When

is

### (Step 1)

Compound [Z-F-D1-603] can be obtained by reacting Compound [Z-F-D1-601] with [Z-F-D1-602] in the presence of a condensation reagent in a solvent under a condition of a commonly-used amide bond formation reaction.

Examples of the solvent for the reaction include toluene, dichloromethane, chloroform, tetrahydrofuran, dioxane, N,N-dimethylformamide, acetonitrile; which may be used alone or as a mixture of two or more.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), carbonyldiimidazole (CDI), and O-(7-azabenzotriazol-1-yl)-N,N,N′,N′-tetramethyluronium hexafluorophosphate (HATU). As necessary, 1-hydroxy-1H-benzotriazole monohydrate (HOBt.H₂O) 4-dimethylaminopyridine (DMAP), N,N-diisopropylethylamine, and the like may be added.

The reaction temperature usually ranges about room temperature to 120° C.

The reaction time usually ranges about 30 minutes to 3 days.

Alternatively, in the above amidation reaction, Compound [Z-F-D1-603] can be prepared by the reaction of an acid halide or mixed acid anhydride of compound [Z-F-D1-601] with compound [Z-F-D1-602].

The acid halide of compound [Z-F-D1-601] can be derived by the reaction of an carboxylic acid of compound [Z-F-D1-601] with thionyl chloride, oxalyl chloride etc. wherein a catalytic amount of N,N-dimethylformamide may be added.

The mixed acid anhydride of compound [Z-F-D1-601] can be derived by the reaction of a carboxylic acid of compound [Z-F-D1-601] with ethyl chlorocarbonate etc.

(Step 2)

The Step 2 is a transformation reaction from an amide to an thioamide. Compound [Z-F-D1-604] can be obtained by reacting Compound [Z-F-D1-603] in the presence of a sulfating agent in a solvent.

Examples of the solvent for the reaction include toluene, dichloromethane, chloroform, tetrahydrofuran, dioxane; which may be used alone or as a mixture of two or more.

Examples of the sulfating agent for the reaction include Lawesson's reagent.

The reaction temperature usually ranges about room temperature to 120° C.

The reaction time usually ranges about 30 minutes to 3 days.

(Step 3)

Compound [Z-F-D1-605] can be obtained by reacting Compound [Z-F-D1-604] with a methylating agent in a solvent.

Examples of the solvent for the reaction include toluene, dichloromethane, chloroform, tetrahydrofuran, dioxane, acetonitrile; which may be used alone or as a mixture of two or more.

Examples of the methylating agent for the reaction include methyl iodide, and trimethyloxonium tetrafluoroborate.

The reaction temperature usually ranges about room temperature to 120° C.

The reaction time usually ranges about 30 minutes to 3 days.

(Step 4)

The Step 4 is an amide bond formation reaction. Compound [Z-F-D1-607] can be obtained by reacting Compound [Z-F-D1-606] with hydrazine monohydrate in the presence of a condensation reagent in a solvent.

Examples of the solvent for the reaction include toluene, dichloromethane, chloroform, tetrahydrofuran, dioxane, N,N-dimethylformamide, acetonitrile; which may be used alone or as a mixture of two or more.

Examples of the condensation reagent for the reaction include water-soluble carbodiimide (WSC.HCl: 1-ethyl-3-(3-dimethylaminopropyl)carbodiimide hydrochloride), and N,N′-dicyclohexylcarbodiimide (DCC). As necessary, 1-hydroxy-1H-benzotriazole monohydrate (HOBt.H₂O), 4-dimethylaminopyridine (DMAP), and the like may be added.

The reaction temperature usually ranges about 0° C. to 100° C.

The reaction time usually ranges about 10 minutes to 3 days.

(Step 5)

Compound [Z-F-D1-608] can be obtained by reacting Compound [Z-F-D1-607] with Compound [Z-F-D1-605] in a solvent.

Examples of the solvent for the reaction include ethanol, dioxane, water; which may be used alone or as a mixture of two or more.

As necessary, sodium acetate, and the like may be added.

(Step 6)

Compound [Z-F-D1-609] can be obtained from Compound [Z-F-D1-608] in a solvent under a commonly-used condition of the ester hydrolysis reaction. The ester hydrolysis reaction may be performed under the alkaline or acidic condition.

Examples of the base for the alkaline condition include an aqueous solution of alkali metal hydroxide such as lithium hydroxide, sodium hydroxide, and potassium hydroxide.

Examples of the acid for the acidic condition include hydrochloric acid, hydrobromic acid, sulfuric acid, and trifluoroacetic acid.

Examples of the solvent for the reaction include toluene, methanol, ethanol, isopropyl alcohol, tetrahydrofuran, dioxane, water; which may be used alone or as a mixture of two or more.

The reaction temperature usually ranges about 0° C. to 120° C.

The reaction time usually ranges about 30 minutes to 3 days.

### Preparation Method 6G-1

When

is

### (Step 1)

Compound [Z-G-D1-602] can be obtained by reacting Compound [Z-G-D1-601] with N,O-dimethylhydroxylamine or a hydrochloride salt thereof in the presence of a condensation reagent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, and acetone; which may be used alone or as a mixture of two or more.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), and carbonyldiimidazole (CDI). As necessary, 1-hydroxy-1H-benzotriazole monohydrate (HOBt.H₂O) 4-dimethylaminopyridine (DMAP), and the like may be added.

The reaction temperature usually ranges about room temperature to 120° C.

The reaction time usually ranges about 30 minutes to 3 days.

(Step 2)

Compound [Z-G-D1-603] can be obtained by reacting Compound [Z-G-D1-602] in the presence of a reducing agent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme; which may be used alone or as a mixture of two or more.

Examples of the reducing agent for the reaction include diisobutylaluminium hydride and lithium aluminium hydride.

The reaction temperature usually ranges about −78° C. to room temperature.

The reaction time usually ranges about 30 minutes to 3 days.

(Step 3)

Compound [Z-G-D1-604] can be obtained by reacting Compound [Z-G-D1-603] in the presence of carbon tetrabromide and triphenylphosphine in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, and diglyme; which may be used alone or as a mixture of two or more.

The reaction temperature usually ranges about −30° C. to 100° C.

The reaction time usually ranges about 30 minutes to 3 days.

(Step 4)

Compound [Z-G-D1-606] can be obtained by reacting Compound [Z-G-D1-605] with N,O-dimethylhydroxylamine or a hydrochloride salt thereof in the presence of a condensation reagent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, and acetone; which may be used alone or as a mixture of two or more.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), and carbonyldiimidazole (CDI). As necessary, 1-hydroxy-1H-benzotriazole monohydrate (HOBt.H₂O) 4-dimethylaminopyridine (DMAP), and the like may be added.

The reaction temperature usually ranges about room temperature to 120° C.

The reaction time usually ranges about 30 minutes to 3 days.

(Step 5)

Compound [Z-G-D1-607] can be obtained by reacting Compound [Z-G-D1-606] with Compound [Z-G-D1-604] in the presence of a base in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme; which may be used alone or as a mixture of two or more.

Examples of the base for the reaction include butyllithium, methyllithium, ethylmagnesium bromide, and lithium diisopropylamide (LDA).

The reaction temperature usually ranges about −78° C. to 50° C.

The reaction time usually ranges about 30 minutes to 3 days.

(Step 6)

Compound [Z-G-D1-608] can be obtained by reacting Compound [Z-G-D1-607] with O-methylhydroxylamine or hydrochloride salt thereof in a solvent.

Examples of the solvent for the reaction include methanol, ethanol, isopropyl alcohol, benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, acetone, water, and pyridine; which may be used alone or as a mixture of two or more.

The reaction temperature usually ranges about −10° C. to 50° C.

The reaction time usually ranges about 30 minutes to 3 days.

(Step 7)

Compound [Z-G-D1-609] can be obtained by the cyclization reaction of Compound [Z-G-D1-608] in the presence of halogen or organohalide in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, and acetone; which may be used alone or as a mixture of two or more.

Examples of the halogen or the organohalide for the reaction include bromine, iodine, N-bromosuccinimide, N-iodosuccinimide, and iodine monochloride.

The reaction temperature usually ranges about −10° C. to 50° C.

The reaction time usually ranges about 30 minutes to 3 days.

(Step 8)

Compound [Z-G-D1-611] can be obtained by reacting Compound [Z-G-D1-609] with Compound [Z-G-D1-610] in the presence of a metal catalyst in a solvent.

As the Compound [Z-G-D1-610], alkylboronic acid, alkylboronic acid ester, alkylzinc reagent, alkylmagnesium reagent, and the like may be used.

Examples of the solvent for the reaction include benzene, toluene, xylene, hexane, diethyl ether, tetrahydrofuran, dioxane, 1,2-dimethoxyethane, diglyme, N,N-dimethylformamide, dimethyl sulfoxide, acetonitrile, acetone, and water; which may be used alone or as a mixture of two or more.

Examples of the metal catalyst for the reaction include a palladium catalyst such as bis(triphenylphosphine)palladium(II) dichloride and 1,1′-bis(diphenylphosphino)ferrocene-palladium(II) dichloride; a nickel catalyst such as [1,2-bis(diphenylphosphino)ethane]nickel(II) dichloride and nickel(II) acetylacetonate; and an iron catalyst such as iron(III) chloride.

As necessary, a base or an inorganic salt may be added. Examples of the base or the inorganic salt for the reaction include tripotassium phosphate, sodium carbonate, potassium carbonate, sodium acetate, and cesium fluoride.

The reaction temperature usually ranges about −10° C. to 150° C.

The reaction time usually ranges about 30 minutes to 3 days.

(Step 9)

Compound [Z-G-D1-612] can be obtained from Compound [Z-G-D1-611] in a solvent under a commonly-used condition of ester hydrolysis reaction.

The ester hydrolysis reaction may be performed under the alkaline or acidic condition.

Examples of the base for the alkaline condition include an aqueous solution of alkali metal hydroxide such as lithium hydroxide, sodium hydroxide, and potassium hydroxide.

Examples of the acid for the acidic condition include hydrochloric acid, hydrobromic acid, sulfuric acid, and trifluoroacetic acid.

Examples of the solvent for the reaction include toluene, methanol, ethanol, isopropyl alcohol, tetrahydrofuran, dioxane, water; which may be used alone or as a mixture of two or more.

The reaction temperature usually ranges about 0° C. to 120° C.

The reaction time usually ranges about 30 minutes to 3 days.

### Preparation Method 6G-2

(Step 1)

Compound [Z-G-D1-602] can be obtained by reacting Compound [Z-G-D1-601] with N,O-dimethylhydroxylamine or a hydrochloride salt thereof in the presence of a condensation reagent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, and acetone; which may be used alone or as a mixture of two or more.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), and carbonyldiimidazole (CDI). As necessary, 1-hydroxy-1H-benzotriazole monohydrate (HOBt.H₂O), 4-dimethylaminopyridine (DMAP), and the like may be added.

The reaction temperature usually ranges about room temperature to 120° C.

The reaction time usually ranges about 30 minutes to 3 days.

(Step 2)

Compound [Z-G-D1-603] can be obtained by reacting Compound [Z-G-D1-602] in the presence of a reducing agent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme; which may be used alone or as a mixture of two or more.

Examples of the reducing agent for the reaction include diisobutylaluminium hydride and lithium aluminium hydride.

The reaction temperature usually ranges about −78° C. to room temperature.

The reaction time usually ranges about 30 minutes to 3 days.

(Step 3)

Compound [Z-G-D1-604] can be obtained by reacting Compound [Z-G-D1-603] in the presence of carbon tetrabromide and triphenylphosphine in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme; which may be used alone or as a mixture of two or more.

The reaction temperature usually ranges about −30° C. to 100° C.

The reaction time usually ranges about 30 minutes to 3 days.

(Step 4)

Compound [Z-G-D1-605] can be obtained by reacting Compound [Z-G-D1-604] with a base in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme; which may be used alone or as a mixture of two or more.

Examples of the base for the reaction include butyllithium, methyllithium, ethylmagnesium bromide, and lithium diisopropylamide (LDA).

The reaction temperature usually ranges about −78° C. to 50° C.

The reaction time usually ranges about 30 minutes to 3 days.

(Step 5)

Compound [Z-G-D1-607] can be obtained by reacting Compound [Z-G-D1-606] with N,O-dimethylhydroxyiamine or a hydrochloride salt thereof in the presence of a condensation reagent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, and acetone; which may be used alone or as a mixture of two or more.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), and carbonyldiimidazole (CDI). As necessary, 1-hydroxy-1H-benzotriazole monohydrate (HOBt.H₂O) 4-dimethylaminopyridine (DMAP), and the like may be added.

The reaction temperature usually ranges about room temperature to 120° C.

The reaction time usually ranges about 30 minutes to 3 days.

(Step 6)

Compound [Z-G-D1-608] can be obtained by reacting Compound [Z-G-D1-607] in the presence of a reducing agent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme; which may be used alone or as a mixture of two or more.

Examples of the reducing agent for the reaction include diisobutylaluminium hydride and lithium aluminium hydride.

The reaction temperature usually ranges about −78° C. to room temperature.

The reaction time usually ranges about 30 minutes to 3 days.

(Step 7)

Compound [Z-G-D1-609] can be obtained by reacting Compound [Z-G-D1-608] with hydroxylamine or a hydrochloride salt thereof in a solvent.

Examples of the solvent for the reaction include benzene, toluene, xylene, hexane, diethyl ether, tetrahydrofuran, dioxane, 1,2-dimethoxyethane, diglyme, methanol, ethanol, acetonitrile, water; which may be used alone or as a mixture of two or more.

When the hydrochloride salt of hydroxylamine is used, examples of the base for the reaction include pyridine, triethylamine, sodium hydroxide, sodium acetate, potassium carbonate, sodium carbonate, and sodium hydrogen carbonate.

The reaction temperature usually ranges about −10° C. to 130° C.

The reaction time usually ranges about 30 minutes to 3 days.

(Step 8)

Compound [Z-G-D1-610] can be obtained by reacting Compound [Z-G-D1-609] in the presence of a chlorinating agent in a solvent.

Examples of the solvent for the reaction include dichloromethane, chloroform, carbon tetrachloride, 1,2-dichloroethane, N,N-dimethylformamide, acetic acid; which may be used alone or as a mixture of two or more.

Examples of the halogenating agent for the reaction include chlorine and N-chlorosuccinimide.

The reaction temperature usually ranges about −10° C. to 100° C.

The reaction time usually ranges about 30 minutes to 3 days.

(Step 9)

Compound [Z-G-D1-611] can be obtained by reacting Compound [Z-G-D1-610] with [Z-G-D1-605] in the presence of a base in a solvent.

Examples of the solvent for the reaction include benzene, toluene, xylene, hexane, diethyl ether, tetrahydrofuran, dioxane, 1,2-dimethoxyethane, diglyme, water; which may be used alone or as a mixture of two or more.

Examples of the base for the reaction include triethylamine, sodium hydroxide, potassium carbonate, sodium carbonate, and sodium hydrogen carbonate.

The reaction temperature usually ranges about 0° C. to 150° C.

The reaction time usually ranges about 30 minutes to 3 days.

(Step 10)

Compound [Z-G-D1-612] can be obtained by reacting Compound [Z-G-D1-611] in the presence of a halogenating agent in a solvent.

Examples of the solvent for the reaction include dichloromethane, chloroform, carbon tetrachloride, 1,2-dichloroethane, N,N-dimethylformamide, acetic acid; which may be used alone or as a mixture of two or more.

Examples of the halogenating agent for the reaction include bromine, iodine, iodine monochloride, N-bromosuccinimide, N-iodosuccinimide, and 1,3-dibromo-5,5-dimethylhydantoin. As necessary, cerium(IV) diammonium nitrate, trifluoroacetic acid, and the like may be added.

The reaction temperature usually ranges about −10° C. to 100° C.

The reaction time usually ranges about 1 hr to 3 days.

(Step 11)

Compound [Z-G-D1-614] can be obtained by reacting Compound [Z-G-D1-612] with Compound [Z-G-D1-613] in the presence of a metal catalyst in a solvent.

As the Compound [Z-G-D1-613], alkylboronic acid, alkylboronic acid ester, alkylzinc reagent, alkylmagnesium reagent, and the like may be used.

Examples of the solvent for the reaction include benzene, toluene, xylene, hexane, diethyl ether, tetrahydrofuran, dioxane, 1,2-dimethoxyethane, diglyme, N,N-dimethylformamide, dimethyl sulfoxide, acetonitrile, acetone, and water; which may be used alone or as a mixture of two or more.

Examples of the metal catalyst for the reaction include a palladium catalyst such as bis(triphenylphosphine)palladium(II) dichloride and 1,1′-bis(diphenylphosphino)ferrocene-palladium(II) dichloride; a nickel catalyst such as [1,2-bis(diphenylphosphino)ethane]nickel(II) dichloride, nickel(II) acetylacetonate; and an iron catalyst such as iron(III) chloride.

As necessary, a base or an inorganic salt may be added. Examples of the base or the inorganic salt for the reaction include tripotassium phosphate, sodium carbonate, potassium carbonate, sodium acetate, and cesium fluoride.

The reaction temperature usually ranges about −10° C. to 150° C.

The reaction time usually ranges about 30 minutes to 3 days.

(Step 12)

Compound [Z-G-D1-615] can be obtained from Compound [Z-G-D1-614] in a solvent under a commonly-used condition of ester hydrolysis reaction.

The ester hydrolysis reaction may be performed under the alkaline or acidic condition.

Examples of the base for the alkaline condition include an aqueous solution of alkali metal hydroxide such as lithium hydroxide, sodium hydroxide, and potassium hydroxide.

Examples of the acid for the acidic condition include hydrochloric acid, hydrobromic acid, sulfuric acid, and trifluoroacetic acid.

Examples of the solvent for the reaction include toluene, methanol, ethanol, isopropyl alcohol, tetrahydrofuran, dioxane, water; which may be used alone or as a mixture of two or more.

The reaction temperature usually ranges about 0° C. to 120° C.

The reaction time usually ranges about 30 minutes to 3 days.

(Outline)

As described below, “compound B” can be prepared from “compound A” by the above preparation methods. Further, “compound B” is transformed to “compound C” by the above Preparation method 5′ (amidation reaction etc.). Specifically, Compound [Z-G-B-612] can be prepared from Compound [Z-G-B-605] in Table 78 by the above preparation methods.

Compound [Z-G-B-612] is transformed to Compound [III-G-B] by the above Preparation method 5′ (amidation reaction etc.).

TABLE 78

| 6G-1 | Compound A | Compound B |
|---|---|---|
| | | |
| | [Z-G-B-605] | [Z-G-B-612] |

| 6G-2 | Compound C |
|---|---|
| | |
| | [III-G-B] |

In a similar way, in Tables 79 to 81, compound B can be prepared from compound A. Compound B is transformed to compound C by Preparation method 5′ etc.

TABLE 79

| 6G-3 | Compound A | Compound B |
|---|---|---|
| | | |
| | [Z-G-C-605] | [Z-G-C-612] |

| 6G-4 | Compound C |
|---|---|
| | |
| | [III-G-C] |

TABLE 80

| 6G-5 | Compound A | Compound B |
|---|---|---|
| | | |
| | [Z-G-D2-605] | [Z-G-D2-612] |

| 6G-6 | Compound C |
|---|---|
| | |
| | [III-G-D2] |

TABLE 81

| | Compound A | Compound B |
|---|---|---|
| 6G-7 | | |
| | [Z-G-D1-606] | [Z-G-D1-609] |

| 6G-8 | Compound C |
|---|---|
| | |
| | [III-G-D1] |

### Preparation Method 6H

When

is

(Step 1)

Compound [Z-H-D1-602] can be obtained by reacting Compound [Z-H-D1-601] with N,O-dimethylhydroxylamine or a hydrochloride salt thereof in the presence of a condensation reagent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, and acetone; which may be used alone or as a mixture of two or more.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), and carbonyldiimidazole (CDI). As necessary, 1-hydroxy-1H-benzotriazole monohydrate (HOBt.H₂O), 4-dimethylaminopyridine (DMAP), and the like may be added.

The reaction temperature usually ranges about room temperature to 120° C.

The reaction time usually ranges about 30 minutes to 3 days.

(Step 2)

Compound [Z-H-D1-603] can be obtained by reacting Compound [Z-H-D1-602] in the presence of a reducing agent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, and diglyme; which may be used alone or as a mixture of two or more.

Examples of the reducing agent for the reaction include diisobutylaluminium hydride and lithium aluminium hydride.

The reaction temperature usually ranges about −78° C. to room temperature.

The reaction time usually ranges about 30 minutes to 3 days.

(Step 3)

Compound [Z-H-D1-604] can be obtained by reacting Compound [Z-H-D1-603] in the presence of carbon tetrabromide and triphenylphosphine in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme; which may be used alone or as a mixture of two or more.

The reaction temperature usually ranges about −30° C. to 100° C.

The reaction time usually ranges about 30 minutes to 3 days.

(Step 4)

Compound [Z-H-D1-605] can be obtained by reacting Compound [Z-H-D1-604] with a base in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme; which may be used alone or as a mixture of two or more.

Examples of the base for the reaction include butyllithium, methyllithium, ethylmagnesium bromide, and lithium diisopropylamide (LDA).

The reaction temperature usually ranges about −78° C. to 50° C.

The reaction time usually ranges about 30 minutes to 3 days.

(Step 5)

Compound [Z-H-D1-607] can be obtained by a reaction of Compound [Z-H-D1-606] with diphenylphosphoryl azide (DPPA) in the presence of a base in a solvent followed by a reaction with trimethylsilyloxy sodium.

Examples of the solvent for the reaction include methylene chloride, chloroform, diethyl ether, tetrahydrofuran, dioxane, 1,2-dimethoxyethane, diglyme, ethyl acetate, methyl acetate, butyl acetate, N,N-dimethylformamide, acetonitrile, and toluene.

Examples of the base for the reaction include an organic base such as triethylamine, N,N-diisopropylethylamine, and pyridine.

The reaction temperature usually ranges about 0° C. to 150° C.

The reaction time usually ranges about 30 minutes to 3 days.

(Step 6)

Compound [Z-H-D1-608] can be obtained by reacting Compound [Z-H-D1-607] with imidazole-1-sulfonyl azide hydrochloride in the presence of a base in a solvent.

Examples of the solvent for the reaction include methanol, ethanol, isopropyl alcohol, methylene chloride, chloroform, carbon tetrachloride, 1,2-dichloroethane, diethyl ether, tetrahydrofuran, dioxane, 1,2-dimethoxyethane, diglyme, ethyl acetate, methyl acetate, butyl acetate, N,N-dimethylformamide, and acetonitrile.

Examples of the base for the reaction include potassium carbonate, and sodium carbonate.

The reaction temperature usually ranges about 0° C. to 150° C.

The reaction time usually ranges about 30 minutes to 3 days.

(Step 7)

Compound [Z-H-D1-609] can be obtained by reacting Compound [Z-H-D1-608] with Compound [Z-H-D1-605] in the presence of copper(I) iodide in a solvent.

Examples of the solvent for the reaction include methanol, ethanol, isopropyl alcohol, tert-butanol, methylene chloride, chloroform, carbon tetrachloride, 1,2-dichloroethane, diethyl ether, tetrahydrofuran, dioxane, 1,2-dimethoxyethane, diglyme, ethyl acetate, methyl acetate, butyl acetate, N,N-dimethylformamide, and acetonitrile.

The reaction temperature usually ranges about 0° C. to 150° C.

The reaction time usually ranges about 30 minutes to 3 days.

(Step 8)

Compound [Z-H-D1-611] can be obtained by reacting Compound [Z-H-D1-609] with Compound [Z-H-D1-610] in the presence of a metal catalyst in a solvent.

As the Compound [Z-H-D1-610], alkylboronic acid, alkylboronic acid ester, alkylzinc reagent, alkylmagnesium reagent, and the like may be used.

Examples of the solvent for the reaction include benzene, toluene, xylene, hexane, diethyl ether, tetrahydrofuran, dioxane, 1,2-dimethoxyethane, diglyme, N,N-dimethylformamide, dimethyl sulfoxide, acetonitrile, acetone, and water; which may be used alone or as a mixture of two or more.

Examples of the metal catalyst for the reaction include a palladium catalyst such as bis(triphenylphosphine)palladium(II) dichloride and 1,1′-bis(diphenylphosphino)ferrocene-palladium(II) dichloride; a nickel catalyst such as [1,2-bis(diphenylphosphino)ethane]nickel(II) dichloride and nickel(II) acetylacetonate; and an iron catalyst such as iron(III) chloride.

As necessary, a base or an inorganic salt may be added. Examples of the base or the inorganic salt for the reaction include tripotassium phosphate, sodium carbonate, potassium carbonate, sodium acetate, and cesium fluoride.

The reaction temperature usually ranges about −10° C. to 150° C.

The reaction time usually ranges about 30 minutes to 3 days.

(Step 9)

Compound [Z-H-D1-612] can be obtained from Compound [Z-H-91-611] in a solvent under a commonly-used condition of ester hydrolysis reaction.

The ester hydrolysis reaction may be performed under the alkaline or acidic condition.

Examples of the base for the alkaline condition include an aqueous solution of alkali metal hydroxide such as lithium hydroxide, sodium hydroxide, and potassium hydroxide.

Examples of the acid for the acidic condition include hydrochloric acid, hydrobromic acid, sulfuric acid, and trifluoroacetic acid.

Examples of the solvent for the reaction include toluene, methanol, ethanol, isopropyl alcohol, tetrahydrofuran, dioxane, water; which may be used alone or as a mixture of two or more.

The reaction temperature usually ranges about 0° C. to 120° C.

The reaction time usually ranges about 30 minutes to 3 days.

(Outline)

As described below, “compound B” can be prepared from “compound A” by the above preparation methods. Further, “compound B” is transformed to “compound C” by the above Preparation method 5′ (amidation reaction etc.). Specifically, Compound [Z-G-B-612] can be prepared from Compound [Z-H-B-605] in Table 78 by the above preparation methods.

Compound [Z-H-B-612] is transformed to Compound [III-H-B] by the above Preparation method 5′ (amidation reaction etc.).

TABLE 82

| | Compound A | Compound B |
|---|---|---|
| 6H-1 | [Z-H-B-606] | [Z-H-B-616] |

| | Compound C |
|---|---|
| 6H-2 | [III-H-B] |

In a similar way, in table 83 to 85, compound B can be prepared from compound A. Compound B is transformed to compound C by Preparation method 5° etc.

TABLE 83

| | Compound A | Compound B |
|---|---|---|
| 6H-3 | [Z-H-C-606] | [Z-H-C-612] |

| | Compound C |
|---|---|
| 6H-4 | [III-H-C] |

TABLE 84

| | Compound A | Compound B |
|---|---|---|
| 6H-5 | [Z-H-D2-606] | [Z-H-D2-612] |

| | Compound C |
|---|---|
| 6H-6 | [III-H-D2] |

TABLE 85

| | Compound A | Compound B |
|---|---|---|
| 6H-7 | [Z-H-D1-606] | [Z-H-D1-612] |

| | Compound C |
|---|---|
| 6H-8 | [III-H-D1] |

### Preparation Method 6J

wherein “n^{jj}” is 0 or 1.

(Step 1)

Compound [Z-J-D1-602] can be obtained by reacting Compound [Z-J-D1-601] with N,O-dimethylhydroxylamine or a hydrochloride salt thereof in the presence of a condensation reagent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, and acetone; which may be used alone or as a mixture of two or more.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), and carbonyldiimidazole (CDI). As necessary, 1-hydroxy-1H-benzotriazole monohydrate (HOBt.H₂O), 4-dimethylaminopyridine (DMAP), and the like may be added.

The reaction temperature usually ranges about room temperature to 120° C.

The reaction time usually ranges about 30 minutes to 3 days.

(Step 2)

Compound [Z-J-D1-603] can be obtained by reacting Compound [Z-J-D1-602] in the presence of a reducing agent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme; which may be used alone or as a mixture of two or more.

Examples of the reducing agent for the reaction include diisobutylaluminium hydride and lithium aluminium hydride.

The reaction temperature usually ranges about −78° C. to room temperature.

The reaction time usually ranges about 30 minutes to 3 days.

(Step 3)

Compound [Z-J-D1-604] can be obtained by reacting Compound [Z-J-D1-603] with hydroxylamine or a hydrochloride salt thereof in a solvent.

Examples of the solvent for the reaction include benzene, toluene, xylene, hexane, diethyl ether, tetrahydrofuran, dioxane, 1,2-dimethoxyethane, diglyme, methanol, ethanol, acetonitrile, water; which may be used alone or as a mixture of two or more.

When the hydrochloride salt of hydroxylamine is used, examples of the base for the reaction include pyridine, triethylamine, sodium hydroxide, sodium acetate, potassium carbonate, sodium carbonate, and sodium hydrogen carbonate.

The reaction temperature usually ranges about −10° C. to 130° C.

The reaction time usually ranges about 30 minutes to 3 days.

(Step 4)

Compound [Z-J-D1-605] can be obtained by reacting Compound [Z-J-D1-604] in the presence of a chlorinating agent in a solvent.

Examples of the solvent for the reaction include dichloromethane, chloroform, carbon tetrachloride, 1,2-dichloroethane, N,N-dimethylformamide, acetic acid; which may be used alone or as a mixture of two or more.

Examples of the halogenating agent for the reaction include chlorine and N-chlorosuccinimide.

The reaction temperature usually ranges about −10° C. to 100° C.

The reaction time usually ranges about 30 minutes to 3 days.

(Step 5)

Compound [Z-J-D1-607] can be obtained by reacting Compound [Z-J-D1-605] with Compound [Z-J-D1-606] in the presence of a base in a solvent followed by a treatment with activated carbon for oxidation.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, acetone, water; which may be used alone or as a mixture of two or more.

Examples of the base for the reaction include potassium carbonate.

The reaction temperature usually ranges about room temperature to 120° C.

The reaction time usually ranges about 30 minutes to 3 days.

(Step 6)

Compound [Z-J-D1-608] can be obtained by reacting Compound [Z-J-D1-607] with sodium tetrahydroborate in the presence of cerium(III) chloride heptahydrate in a solvent.

Examples of the solvent for the reaction include benzene, toluene, xylene, hexane, diethyl ether, tetrahydrofuran, dioxane, 1,2-dimethoxyethane, diglyme, methanol, ethanol; which may be used alone or as a mixture of two or more.

The reaction temperature usually ranges about 0° C. to 120° C.

The reaction time usually ranges about 30 minutes to 3 days.

(Step 7)

Compound [Z-J-D1-610] can be obtained by reacting Compound [Z-J-D1-608] with Compound [Z-J-D1-609] in the presence of 4-dimethylaminopyridine (DMAP) in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, and acetone; which may be used alone or as a mixture of two or more.

The reaction temperature usually ranges about room temperature to 120° C.

The reaction time usually ranges about 30 minutes to 3 days.

(Step 8)

Compound [Z-J-D1-611] can be obtained by reacting Compound [Z-J-D1-610] with tri-n-butyltin hydride in the presence of 2,2′-azobis(isobutyronitrile) (AIBN) in a solvent.

Examples of the solvent for the reaction include benzene, toluene, xylene, hexane, diethyl ether, tetrahydrofuran, dioxane, 1,2-dimethoxyethane, diglyme; which may be used alone or as a mixture of two or more.

The reaction temperature usually ranges about room temperature to 120° C.

The reaction time usually ranges about 30 minutes to 3 days.

(Step 9)

Compound [Z-J-D1-612] can be obtained from Compound [Z-J-D1-611] in a solvent under a commonly-used condition of ester hydrolysis reaction.

The ester hydrolysis reaction may be performed under the alkaline or acidic condition.

Examples of the base for the alkaline condition include an aqueous solution of alkali metal hydroxide such as lithium hydroxide, sodium hydroxide, and potassium hydroxide.

Examples of the acid for the acidic condition include hydrochloric acid, hydrobromic acid, sulfuric acid, and trifluoroacetic acid.

Examples of the solvent for the reaction include toluene, methanol, ethanol, isopropyl alcohol, tetrahydrofuran, dioxane, water; which may be used alone or as a mixture of two or more.

The reaction temperature usually ranges about 0° C. to 120° C.

The reaction time usually ranges about 30 minutes to 3 days.

(Outline)

As described below, “compound B” can be prepared from “compound A” by the above preparation methods. Further, “compound B” is transformed to “compound C” by the above Preparation method 5′ (amidation reaction etc.). Specifically, in Table 86, Compound [Z-J-B-612]] can be prepared from Compound [Z-J-B-601] by the above preparation methods.

Compound [Z-G-B-612] is transformed to Compound [III-J-B] by the above Preparation method 5′ (amidation reaction etc.).

TABLE 86

| | Compound A | Compound B |
|---|---|---|
| 6J-1 | [Z-J-B-601] | [Z-J-B-612] |

| | Compound C |
|---|---|
| 6J-2 | [III-J-B] |

In a similar way, in Tables 87 to 89, compound B can be prepared from compound A. Compound B is transformed to compound C by Preparation method 5′ etc.

TABLE 87

| | Compound A | Compound B |
|---|---|---|
| 6J-3 | [Z-J-C-601] | [Z-J-C-612] |

| | Compound C |
|---|---|
| 6J-4 | [III-J-C] |

TABLE 88

| | Compound A | Compound B |
|---|---|---|
| 6J-5 | [Z-J-D2-601] | [Z-J-D2-612] |

| | Compound C |
|---|---|
| 6J-6 | [III-J-D2] |

TABLE 89

| | Compound A | Compound B |
|---|---|---|
| 6J-7 | [Z-J-D1-601] | [Z-J-D1-609] |

| | Compound C |
|---|---|
| 6J-8 | [III-J-D1] |

### Preparation Method 6K

When

is

(Step 1)

Compound [Z-K-D1-602] can be obtained by reacting Compound [Z-K-D1-601] with diphenylphosphoryl azide (DPPA) and an alcohol in the presence of a base in a solvent.

Examples of the solvent for the reaction include methanol, ethanol, isopropyl alcohol, tert-butanol, methylene chloride, chloroform, carbon tetrachloride, 1,2-dichloroethane, diethyl ether, tetrahydrofuran, dioxane, 1,2-dimethoxyethane, diglyme, ethyl acetate, methyl acetate, butyl acetate, N,N-dimethylformamide, and acetonitrile.

Examples of the base for the reaction include an organic base such as triethylamine, N,N-diisopropylethylamine, and pyridine.

Examples of the alcohol for the reaction include methanol, ethanol, isopropyl alcohol, tert-butanol, and benzyl alcohol.

The reaction temperature usually ranges about 0° C. to 150° C.

The reaction time usually ranges about 30 minutes to 3 days.

(Step 2)

Compound [Z-K-D1-603] can be obtained by removal of the protecting group P^{N1} from Compound [Z-K-D1-602] in the presence of an acid in a solvent.

Examples of the solvent for the reaction include methanol, ethanol, isopropyl alcohol, methylene chloride, chloroform, carbon tetrachloride, 1,2-dichloroethane, diethyl ether, tetrahydrofuran, dioxane, 1,2-dimethoxyethane, diglyme, ethyl acetate, methyl acetate, butyl acetate, N,N-dimethylformamide, acetonitrile, acetic acid, and water.

Examples of the acid for the reaction include hydrochloric acid, hydrobromic acid, sulfuric acid, and trifluoroacetic acid.

The reaction temperature usually ranges about 0° C. to 150° C.

The reaction time usually ranges about 30 minutes to 3 days.

(Step 3)

Compound [Z-K-D1-604] can be obtained by reacting Compound [Z-K-D1-603] with imidazole-1-sulfonyl azide hydrochloride in the presence of a base in a solvent.

Examples of the solvent for the reaction include methanol, ethanol, isopropyl alcohol, methylene chloride, chloroform, carbon tetrachloride, 1,2-dichloroethane, diethyl ether, tetrahydrofuran, dioxane, 1,2-dimethoxyethane, diglyme, ethyl acetate, methyl acetate, butyl acetate, N,N-dimethylformamide, and acetonitrile.

Examples of the base for the reaction include potassium carbonate, and sodium carbonate.

The reaction temperature usually ranges about 0° C. to 150° C.

The reaction time usually ranges about 30 minutes to 3 days.

(Step 4)

Compound [Z-K-D1-606] can be obtained by reacting Compound [Z-K-D1-605] with N,O-dimethylhydroxylamine or a hydrochloride salt thereof in the presence of a condensation reagent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, and acetone; which may be used alone or as a mixture of two or more.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), and carbonyldiimidazole (CDI). As necessary, 1-hydroxy-1H-benzotriazole monohydrate (HOBt.H₂O), 4-dimethylaminopyridine (DMAP), and the like may be added.

The reaction temperature usually ranges about room temperature to 120° C.

The reaction time usually ranges about 30 minutes to 3 days.

(Step 5)

Compound [Z-K-D1-607] can be obtained by reacting Compound [Z-K-D1-606] in the presence of a reducing agent in a solvent. Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme; which may be used alone or as a mixture of two or more.

Examples of the reducing agent for the reaction include diisobutylaluminium hydride and lithium aluminium hydride.

The reaction temperature usually ranges about −78° C. to room temperature.

The reaction time usually ranges about 30 minutes to 3 days.

(Step 6)

Compound [Z-K-D1-608] can be obtained by reacting Compound [Z-K-D1-607] with dimethyl(1-diazo-2-oxopropyl)phosphonate in a solvent.

Examples of the solvent for the reaction include methanol, ethanol, isopropyl alcohol, tert-butanol, methylene chloride, chloroform, carbon tetrachloride, 1,2-dichloroethane, diethyl ether, tetrahydrofuran, dioxane, 1,2-dimethoxyethane, diglyme, ethyl acetate, methyl acetate, butyl acetate, N,N-dimethylformamide, and acetonitrile.

The reaction temperature usually ranges about 0° C. to 150° C.

The reaction time usually ranges about 30 minutes to 3 days.

(Step 7)

Compound [Z-K-D1-609] can be obtained by reacting Compound [Z-K-D1-608] with Compound [Z-K-D1-604] in a solvent.

Examples of the solvent for the reaction include methanol, ethanol, isopropyl alcohol, tert-butanol, methylene chloride, chloroform, carbon tetrachloride, 1,2-dichloroethane, diethyl ether, tetrahydrofuran, dioxane, 1,2-dimethoxyethane, diglyme, ethyl acetate, methyl acetate, butyl acetate, N,N-dimethylformamide, and acetonitrile.

The reaction temperature usually ranges about 0° C. to 150° C.

The reaction time usually ranges about 30 minutes to 3 days.

(Step 8)

Compound [Z-K-D1-611] can be obtained by reacting Compound [Z-K-D1-609] with Compound [Z-K-D1-610] in the presence of a metal catalyst in a solvent.

As the Compound [Z-K-D1-610], alkylboronic acid, alkylboronic acid ester, alkylzinc reagent, alkylmagnesium reagent, and the like may be used.

Examples of the solvent for the reaction include benzene, toluene, xylene, hexane, diethyl ether, tetrahydrofuran, dioxane, 1,2-dimethoxyethane, diglyme, N,N-dimethylformamide, dimethyl sulfoxide, acetonitrile, acetone, and water; which may be used alone or as a mixture of two or more.

Examples of the metal catalyst for the reaction include a palladium catalyst such as bis(triphenylphosphine)palladium(II) dichloride and 1,1′-bis(diphenylphosphino)ferrocene-palladium(II) dichloride; a nickel catalyst such as [1,2-bis(diphenylphosphino)ethane]nickel(II) dichloride and nickel(II) acetylacetonate; and an iron catalyst such as iron(III) chloride.

As necessary, a base or an inorganic salt may be added. Examples of the base or the inorganic salt for the reaction include tripotassium phosphate, sodium carbonate, potassium carbonate, sodium acetate, and cesium fluoride.

The reaction temperature usually ranges about −10° C. to 150° C.

The reaction time usually ranges about 30 minutes to 3 days.

(Step 9)

Compound [Z-K-D1-612] can be obtained from Compound [Z-K-D1-611] in a solvent under a commonly-used condition of ester hydrolysis reaction.

The ester hydrolysis reaction may be performed under the alkaline or acidic condition.

Examples of the base for the alkaline condition include an aqueous solution of alkali metal hydroxide such as lithium hydroxide, sodium hydroxide, and potassium hydroxide.

Examples of the acid for the acidic condition include hydrochloric acid, hydrobromic acid, sulfuric acid, and trifluoroacetic acid.

Examples of the solvent for the reaction include toluene, methanol, ethanol, isopropyl alcohol, tetrahydrofuran, dioxane, water; which may be used alone or as a mixture of two or more.

The reaction temperature usually ranges about 0° C. to 120° C.

The reaction time usually ranges about 30 minutes to 3 days.

(Outline)

As described below, “compound B” can be prepared from “compound A” by the above preparation methods. Further, “compound B” is transformed to “compound C” by the above Preparation method 5′ (amidation reaction etc.). Specifically, in Table 90, Compound [Z-K-B-612] can be prepared from Compound [Z-K-B-606] by the above preparation methods.

Compound [Z-K-B-612] is transformed to Compound [III-K-B] by the above Preparation method 5′ (amidation reaction etc.).

TABLE 90

| | Compound A | Compound B |
|---|---|---|
| 6K-1 | [Z-K-B-605] | [Z-K-B-612] |

| | Compound C |
|---|---|
| 6K-2 | [III-K-B] |

In a similar way, in Tables 91 to 93, compound B can be prepared from compound A. Compound B is transformed to compound C by Preparation method 5′ etc.

TABLE 91

| | Compound A | Compound B |
|---|---|---|
| 6K-3 | [Z-K-C-605] | [Z-K-C-612] |

| | Compound C |
|---|---|
| 6K-4 | [III-K-C] |

TABLE 92

| | Compound A | Compound B |
|---|---|---|
| 6K-5 | [Z-K-D2-605] | [Z-K-D2-612] |

| | Compound C |
|---|---|
| 6K-6 | [III-K-D2] |

TABLE 93

| | Compound A | Compound B |
|---|---|---|
| 6K-7 | [Z-K-D1-605] | [Z-K-D1-612] |

| | Compound C |
|---|---|
| 6K-8 | [III-K-D1] |

### Preparation Method 6L

When

is

(Step 1)

Compound [Z-L-D1-602] can be obtained by reacting Compound [Z-L-D1-601] with N,O-dimethylhydroxylamine or a hydrochloride salt thereof in the presence of a condensation reagent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, and acetone; which may be used alone or as a mixture of two or more.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), and carbonyldiimidazole (CDI). As necessary, 1-hydroxy-1H-benzotriazole monohydrate (HOBt.H₂O), 4-dimethylaminopyridine (DMAP), and the like may be added.

The reaction temperature usually ranges about room temperature to 120° C.

The reaction time usually ranges about 30 minutes to 3 days.

(Step 2)

Compound [Z-L-D1-603] can be obtained by reacting Compound [Z-L-D1-602] in the presence of a reducing agent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme; which may be used alone or as a mixture of two or more.

Examples of the reducing agent for the reaction include diisobutylaluminium hydride and lithium aluminium hydride.

The reaction temperature usually ranges about −78° C. to room temperature.

The reaction time usually ranges about 30 minutes to 3 days.

(Step 3)

Compound [Z-L-D1-604] can be obtained by reacting Compound [Z-L-D1-603] with hydroxylamine or a hydrochloride salt thereof in a solvent.

Examples of the solvent for the reaction include benzene, toluene, xylene, hexane, diethyl ether, tetrahydrofuran, dioxane, 1,2-dimethoxyethane, diglyme, methanol, ethanol, acetonitrile, water; which may be used alone or as a mixture of two or more.

When the hydrochloride salt of hydroxylamine is used, examples of the base for the reaction include pyridine, triethylamine, sodium hydroxide, sodium acetate, potassium carbonate, sodium carbonate, and sodium hydrogen carbonate.

The reaction temperature usually ranges about −10° C. to 130° C.

The reaction time usually ranges about 30 minutes to 3 days.

(Step 4)

Compound [Z-L-D1-605] can be obtained by reacting Compound [Z-L-D1-604] in the presence of a chlorinating agent in a solvent.

Examples of the solvent for the reaction include dichloromethane, chloroform, carbon tetrachloride, 1,2-dichloroethane, N,N-dimethylformamide, acetic acid; which may be used alone or as a mixture of two or more.

Examples of the halogenating agent for the reaction include chlorine and N-chlorosuccinimide.

The reaction temperature usually ranges about −10° C. to 100° C.

The reaction time usually ranges about 30 minutes to 3 days.

(Step 5)

Compound [Z-L-D1-607] can be obtained by reacting Compound [Z-L-D1-606] with N,O-dimethylhydroxylamine or a hydrochloride salt thereof in the presence of a condensation reagent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, and acetone; which may be used alone or as a mixture of two or more.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), and carbonyldiimidazole (CDI). As necessary, 1-hydroxy-1H-benzotriazole monohydrate (HOBt.H₂O), 4-dimethylaminopyridine (DMAP), and the like may be added.

The reaction temperature usually ranges about room temperature to 120° C.

The reaction time usually ranges about 30 minutes to 3 days.

(Step 6)

Compound [Z-L-D1-608] can be obtained by reacting Compound [Z-L-D1-607] in the presence of a reducing agent in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme; which may be used alone or as a mixture of two or more.

Examples of the reducing agent for the reaction include diisobutylaluminium hydride and lithium aluminium hydride.

The reaction temperature usually ranges about −78° C. to room temperature.

The reaction time usually ranges about 30 minutes to 3 days.

(Step 7)

Compound [Z-L-D1-609] can be obtained by reacting Compound [Z-L-D1-608] with dimethyl(1-diazo-2-oxopropyl)phosphonate in a solvent.

Examples of the solvent for the reaction include methanol, ethanol, isopropyl alcohol, tert-butanol, methylene chloride, chloroform, carbon tetrachloride, 1,2-dichloroethane, diethyl ether, tetrahydrofuran, dioxane, 1,2-dimethoxyethane, diglyme, ethyl acetate, methyl acetate, butyl acetate, N,N-dimethylformamide, and acetonitrile.

The reaction temperature usually ranges about 0° C. to 150° C.

The reaction time usually ranges about 30 minutes to 3 days.

(Step 8)

Compound [Z-L-D1-610] can be obtained by reacting Compound [Z-L-D1-609] with [Z-L-D1-605] in the presence of a base in a solvent.

Examples of the solvent for the reaction include benzene, toluene, xylene, hexane, diethyl ether, tetrahydrofuran, dioxane, 1,2-dimethoxyethane, diglyme, water; which may be used alone or as a mixture of two or more.

Examples of the base for the reaction include triethylamine, sodium hydroxide, potassium carbonate, sodium carbonate, and sodium hydrogen carbonate.

The reaction temperature usually ranges about 0° C. to 150° C.

The reaction time usually ranges about 30 minutes to 3 days.

(Step 9)

Compound [Z-G-D1-611] can be obtained by reacting Compound [Z-L-D1-610] in the presence of a halogenating agent in a solvent.

Examples of the solvent for the reaction include dichloromethane, chloroform, carbon tetrachloride, 1,2-dichloroethane, N,N-dimethylformamide, acetic acid; which may be used alone or as a mixture of two or more.

Examples of the halogenating agent for the reaction include bromine, iodine, iodine monochloride, N-bromosuccinimide, N-iodosuccinimide, and 1,3-dibromo-5,5-dimethylhydantoin. As necessary, cerium(IV) diammonium nitrate, trifluoroacetic acid, and the like may be added.

The reaction temperature usually ranges about −10° C. to 100° C.

The reaction time usually ranges about 1 hr to 3 days.

(Step 10)

Compound [Z-L-D1-613] can be obtained by reacting Compound [Z-L-D1-611] with Compound [Z-L-D1-612] in the presence of a metal catalyst in a solvent.

As the Compound [Z-L-D1-612], alkylboronic acid, alkylboronic acid ester, alkylzinc reagent, alkylmagnesium reagent, and the like may be used.

Examples of the solvent for the reaction include benzene, toluene, xylene, hexane, diethyl ether, tetrahydrofuran, dioxane, 1,2-dimethoxyethane, diglyme, N,N-dimethylformamide, dimethyl sulfoxide, acetonitrile, acetone, and water; which may be used alone or as a mixture of two or more.

Examples of the metal catalyst for the reaction include a palladium catalyst such as bis(triphenylphosphine)palladium(II) dichloride and 1,1′-bis(diphenylphosphino)ferrocene-palladium(II) dichloride; a nickel catalyst such as [1,2-bis(diphenylphosphino)ethane]nickel(II) dichloride and nickel(II) acetylacetonate; and an iron catalyst such as iron(III) chloride.

As necessary, a base or an inorganic salt may be added. Examples of the base or the inorganic salt for the reaction include tripotassium phosphate, sodium carbonate, potassium carbonate, sodium acetate, and cesium fluoride.

The reaction temperature usually ranges about −10° C. to 150° C.

The reaction time usually ranges about 30 minutes to 3 days.

(Step 11)

Compound [Z-L-D1-614] can be obtained from Compound [Z-L-D1-613] in a solvent under a commonly-used condition of ester hydrolysis reaction.

The ester hydrolysis reaction may be performed under the alkaline or acidic condition.

Examples of the base for the alkaline condition include an aqueous solution of alkali metal hydroxide such as lithium hydroxide, sodium hydroxide, and potassium hydroxide.

Examples of the acid for the acidic condition include hydrochloric acid, hydrobromic acid, sulfuric acid, and trifluoroacetic acid.

Examples of the solvent for the reaction include toluene, methanol, ethanol, isopropyl alcohol, tetrahydrofuran, dioxane, water; which may be used alone or as a mixture of two or more.

The reaction temperature usually ranges about 0° C. to 120° C.

The reaction time usually ranges about 30 minutes to 3 days.

(Outline)

As described below, “compound B” can be prepared from “compound A” by the above preparation methods. Further, “compound B” is transformed to “compound C” by the above Preparation method 5° (amidation reaction etc.). Specifically, in Table 94, Compound [Z-L-B-614]] can be prepared from Compound [Z-L-B-606] by the above preparation methods.

Compound [Z-L-B-614] is transformed to Compound [III-L-B] by the above Preparation method 5′ (amidation reaction etc.).

TABLE 94

| | Compound A | Compound B |
|---|---|---|
| 6L-1 | [Z-L-B-606] | [Z-L-B-614] |

| | Compound C |
|---|---|
| 6L-2 | [III-L-B] |

In a similar way, in Tables 95 to 97, compound B can be prepared from compound A. Compound B is transformed to compound C by Preparation method 5′ etc.

TABLE 95

| | Compound A | Compound B |
|---|---|---|
| 6L-3 | [Z-L-C-606] | [Z-L-C-614] |

| | Compound C |
|---|---|
| 6L-4 | [III-L-C] |

TABLE 96

| | Compound A | Compound B |
|---|---|---|
| 6L-5 | [Z-L-D2-606] | [Z-L-D2-614] |

| | Compound C |
|---|---|
| 6L-6 | [III-L-D2] |

TABLE 97

| | Compound A | Compound B |
|---|---|---|
| 6L-7 | | |
| | [Z-L-D1-606] | [Z-L-D1-609] |

| 6L-8 | Compound C |
|---|---|
| | |
| | [III-L-D1] |

### Preparation Method 7

### Synthesis Process of [III-X-D2], [III-J-D2], [III-X-D1], and [III-J-D1]

### Preparation Method 7-1 (Another Synthesis Process)

(Step 1)

Compound [Z-X-D2-703] can be obtained by reacting Compound [Z-X-D2-701] with AUX-H [Z-X-D2-702] in the presence of a condensation reagent in a solvent.

Examples of the solvent for the reaction include benzene, toluene, xylene, hexane, dichloromethane, chloroform, tetrahydrofuran, dioxane, 1,2-dimethoxyethane, N,N-dimethylformamide, dimethyl sulfoxide, acetonitrile, and acetone; which may be used alone or as a mixture of two or more.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), and carbonyldiimidazole (CDI). As necessary, 1-hydroxy-1H-benzotriazole monohydrate (HOBt.H₂O), 4-dimethylaminopyridine (DMAP), and the like may be added.

The reaction temperature usually ranges about room temperature to 120° C.

The reaction time usually ranges about 30 minutes to 3 days.

(Step 2)

Compound [Z-X-D2-705] can be obtained by reacting Compound [Z-X-D2-703] with [Z-X-D2-704] in the presence of a base in a solvent.

Examples of the solvent for the reaction include benzene, toluene, xylene, hexane, diethyl ether, tetrahydrofuran, dioxane, 1,2-dimethoxyethane, diglyme; which may be used alone or as a mixture of two or more.

Examples of the base for the reaction include sodium hexamethyldisilazide, lithium hexamethyldisilazide, and lithium diisopropylamide (LDA).

The reaction temperature usually ranges about −78° C. to 50° C.

The reaction time usually ranges about 30 minutes to 3 days.

(Step 3)

Compound [Z-X-D2-706] can be obtained by hydrolysis reaction of Compound [Z-X-D2-705] in a solvent.

The hydrolysis reaction may be performed under the commonly-used condition, for example, under the alkaline condition.

Examples of the base for the reaction include lithium hydroxide, sodium hydroxide, potassium hydroxide, lithium peroxide, potassium peroxide, and sodium peroxide.

Examples of the solvent for the reaction include benzene, toluene, xylene, hexane, methanol, ethanol, isopropyl alcohol, tert-butanol, dichloromethane, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, acetone, water; which may be used alone or as a mixture of two or more.

The reaction temperature usually ranges about −30° C. to 80° C., preferably about 0° C. to room temperature.

The reaction time usually ranges about 30 minutes to 3 days, preferably about 30 minutes to 1 day.

(Supplementary Step)

For example, the following scheme is included.

The same applies to the following 4,5,6,7-tetrahydro-benz[d]isoxazole.

### Preparation Method 7-2

(Step 1)

Compound [Z-X-D1-H-D2-H-703] can be obtained by reacting Compound [Z-X-D1-H-D2-H-701] with AUX-H[Z-X-D1-H-D2-H-702] in the presence of a condensation reagent in a solvent.

Examples of the solvent for the reaction include benzene, toluene, xylene, hexane, dichloromethane, chloroform, tetrahydrofuran, dioxane, 1,2-dimethoxyethane, N,N-dimethylformamide, dimethyl sulfoxide, acetonitrile, and acetone; which may be used alone or as a mixture of two or more.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), and carbonyldiimidazole (CDI). As necessary, 1-hydroxy-1H-benzotriazole monohydrate (HOBt.H₂O) 4-dimethylaminopyridine (DMAP), and the like may be added.

The reaction temperature usually ranges about room temperature to 120° C.

The reaction time usually ranges about 30 minutes to 3 days.

(Step 2)

Compound [Z-X-D1-H-D2-H-705] can be obtained by reacting Compound [Z-X-D1-H-D2-H-703] with [Z-X-D1-H-D2-H-704] in the presence of a base in a solvent.

Examples of the solvent for the reaction include benzene, toluene, xylene, hexane, diethyl ether, tetrahydrofuran, dioxane, 1,2-dimethoxyethane, diglyme; which may be used alone or as a mixture of two or more.

Examples of the base for the reaction include sodium hexamethyldisilazide, lithium hexamethyldisilazide, and lithium diisopropylamide (LDA).

The reaction temperature usually ranges about −78° C. to 50° C.

The reaction time usually ranges about 30 minutes to 3 days.

A reference for the above reaction is (J. Org. Chem. 1999, 64, 6411-6417).

(Step 3)

Compound [Z-X-D1-O-D2-O-701] can be obtained by hydrolysis reaction of Compound [Z-X-D1-H-D2-H-705] in a solvent.

The hydrolysis reaction may be performed under the commonly-used condition, for example, under the alkaline condition.

Examples of the base for the reaction include lithium hydroxide, sodium hydroxide, potassium hydroxide, lithium peroxide, potassium peroxide, and sodium peroxide.

Examples of the solvent for the reaction include benzene, toluene, xylene, hexane, methanol, ethanol, isopropyl alcohol, tert-butanol, dichloromethane, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, acetone, water; which may be used alone or as a mixture of two or more.

The reaction temperature usually ranges about −30° C. to 80° C.

The reaction time usually ranges about 30 minutes to 3 days.

For example, the following scheme is included.

### Preparation Method 7-3 (Synthesis of Stereoisomer)

The following “R^{f}” is C₁₋₆ alkyl group.

(Step 1)

Compound [Z-X-D1-H-D2-H-703] can be obtained by reacting Compound [Z-X-D1-H-D2-H-701] with AUX-H[Z-X-D1-H-D2-H-702] in the presence of a condensation reagent in a solvent.

Examples of the solvent for the reaction include benzene, toluene, xylene, hexane, dichloromethane, chloroform, tetrahydrofuran, dioxane, 1,2-dimethoxyethane, N,N-dimethylformamide, dimethyl sulfoxide, acetonitrile, and acetone; which may be used alone or as a mixture of two or more.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (APPA), and carbonyldiimidazole (CDI). As necessary, 1-hydroxy-1H-benzotriazole monohydrate (HOBt.H₂O), 4-dimethylaminopyridine (DMAP), and the like may be added.

The reaction temperature usually ranges about room temperature to 120° C.

The reaction time usually ranges about 30 minutes to 3 days.

(Step 2)

Compound [Z-X-D1-H-D2-H-705] can be obtained by reacting Compound [Z-X-D1-H-D2-H-703] with Compound [Z-X-D1-H-D2-H-704] in the presence of a base in a solvent.

Examples of the solvent for the reaction include benzene, toluene, xylene, hexane, diethyl ether, tetrahydrofuran, dioxane, 1,2-dimethoxyethane, diglyme; which may be used alone or as a mixture of two or more.

Examples of the base for the reaction include sodium hexamethyldisilazide, lithium hexamethyldisilazide, and lithium diisopropylamide (LDA).

The reaction temperature usually ranges about −78° C. to 50° C.

The reaction time usually ranges about 30 minutes to 3 days.

(Step 3)

Compound [Z-X-D1-O-D2-O-701] can be obtained by hydrolysis reaction of Compound [Z-X-D1-H-D2-H-705] in a solvent.

The hydrolysis reaction may be performed under the commonly-used condition, for example, under the alkaline condition.

Examples of the base for the reaction include lithium hydroxide, sodium hydroxide, potassium hydroxide, lithium peroxide, potassium peroxide, and sodium peroxide.

Examples of the solvent for the reaction include benzene, toluene, xylene, hexane, methanol, ethanol, isopropyl alcohol, tert-butanol, dichloromethane, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, acetone, water; which may be used alone or as a mixture of two or more.

The reaction temperature usually ranges about −30° C. to 80° C.

The reaction time usually ranges about 30 minutes to 3 days.

(Step 4)

Compound [Z-X-D1-O-D2-O-701] can be obtained by reacting Compound [Z-X-D1-O-D2-H-706] with Compound [Z-X-D1-H-D2-H-707] in the presence of lithium diisopropylamide (LDA) in a solvent.

Examples of the solvent for the reaction include benzene, toluene, xylene, hexane, diethyl ether, tetrahydrofuran, dioxane, 1,2-dimethoxyethane, diglyme; which may be used alone or as a mixture of two or more.

The reaction temperature usually ranges about −78° C. to 50° C.

The reaction time usually ranges about 30 minutes to 3 days.

(Supplementary Step)

Compound [III-X-D1-O-D2-O-701] can be obtained from Compound [Z-X-D1-O-D2-O-701] by Preparation method 5′, Preparation method 6, and the like.

### Separation of Diastereomer (Intermediate, Product)

For example, silica-gel column chromatography, recrystallization, and the like are used for the separation. Separation of Racemic form (intermediate, product)

Each optically-active compound can be obtained by sepalating a racemic form by high-performance liquid chromatograph with a chiral stationary phase column.

For example, the condition is as follows:

Instrument: Recycle preparative HPLC LC908 type (Japan Analytical Industry)
Column: DAICEL CHIRALPAK AD 20 mmφ×250 mm
Column temperature: room temperature
Mobile phase: ethanol
Flow rate: 0.5 ml/min

### Detection: UV (254 nm).

Each isomer can be obtained by the above process.

### Preparation Method 8

### Synthesis Process of [III-X-C] and [III-J-C]

Each step of the above Preparation method 8 is as follows.

### Preparation Method 8-1 (Ring-Forming Step)

can be performed according to Preparation method 6.

### Preparation Method 8-2 (Amide-Formation Step)

can be performed according to Preparation method 5′.

(Supplementary)

When “R^{w}” has carbonyl group, a protection of the carbonyl group is appropriately selected.

For example, when “R^{w}” is “—(CH₂)₂—C(═O)—OH”,

“R^{w}” protected by a protecting group includes “—(CH₂)₂—C(═O)—OCH₃” and the like.

### Preparation Method 8-3 (Example of Preparation Method)

For example, the product (right column) can be prepared from the intermediate (left column) in a similar way to the above Preparation methods.

TABLE 98

| | Intermediate | Product |
|---|---|---|
| 8-1 | [Z-X-C3-801] | |
| | | [III-X-C3] |
| 8-2 | [Z-X-C4-801] | |
| | | [III-X-C4] |
| 8-3 | [Z-X-C5-801] | |
| | | [III-X-C5] |
| 8-4 | [Z-X-C6-801] | |
| | | [III-X-C6] |

TABLE 99

| | Intermediate | Product |
|---|---|---|
| 8-5 | [Z-X-N5-801] | |
| | | [III-X-N5] |
| 8-6 | [Z-X-N5O-801] | |
| | | [III-X-N5O] |
| 8-7 | [Z-X-N5O-801] | |
| | | [III-X-N5O] |
| 8-8 | [Z-X-N61-801] | |
| | | [III-X-N61] |
| 8-9 | [Z-X-N62-801] | |
| | | [III-X-N62] |

TABLE 100

| | Intermediate | Product |
|---|---|---|
| 8-10 | [Z-X-CO-801] | |
| | | [III-X-CO] |
| 8-11 | [Z-X-CDU-801] | |
| | | [III-X-CDU] |
| 8-12 | [Z-X-CUU-801] | |
| | | [III-X-CUU] |

TABLE 101

| | | |
|---|---|---|
| 8-13 | [Z-X-CUD-801] | |
| | | [III-X-CUD] |
| 8-14 | [Z-X-CDD-801] | |
| | | [III-X-CDD] |

### Preparation Method 9

### Synthesis Process of “Ring P”

### Preparation Method 9-1

(Step 1)

Compound [III-X-D1-901] can be obtained by reacting Compound [Z-X-D1-901] with azidotrimethyltin or sodium azide in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, N,N-dimethylacetamide, dimethyl sulfoxide, acetone, water; which may be used alone or as a mixture of two or more.

The reaction temperature usually ranges about room temperature to 180° C.

The reaction time usually ranges about 30 minutes to 3 days.

### Preparation Method 9-2

(Step 1)

Compound [Z-X-D1-902-2] can be obtained by reacting Compound [Z-X-D1-902] with hydroxylamine or a hydrochloride salt thereof in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, ethanol, methanol; which may be used alone or as a mixture of two or more.

The reaction temperature usually ranges about room temperature to 120° C.

The reaction time usually ranges about 30 minutes to 3 days.

(Step 2)

Compound [III-X-D1-902] can be obtained by reacting Compound [Z-X-D1-902-2] with N,N′-carbonyldiimidazole (CDI) in the presence of a base in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, ethanol, methanol; which may be used alone or as a mixture of two or more.

Examples of the base for the reaction include 1,8-diazabicyclo[5.4.0]undec-7-ene (DBU).

The reaction temperature usually ranges about 0° C. to 120° C.

The reaction time usually ranges about 30 minutes to 3 days.

### Preparation Method 9-3

(Step 1)

Compound [Z-X-D1-903-2] can be obtained by reacting Compound [Z-X-D1-903] with bromoethyl acetate in the presence of a base in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile; which may be used alone or as a mixture of two or more.

Examples of the base for the reaction include triethylamine, and N,N-diisopropylethylamine.

The reaction temperature usually ranges about room temperature to 120° C.

The reaction time usually ranges about 30 minutes to 3 days.

(Step 2)

Compound [Z-X-D1-903-3] can be obtained by reacting Compound [Z-X-D1-903-2] with potassium cyanate in a solvent.

Examples of the solvent for the reaction include benzene, toluene, xylene, hexane, methylene chloride, chloroform, carbon tetrachloride, 1,2-dichloroethane, diethyl ether, tetrahydrofuran, dioxane, 1,2-dimethoxyethane, diglyme, N,N-dimethylformamide, dimethyl sulfoxide, acetonitrile, ethanol, methanol, and acetic acid; which may be used alone or as a mixture of two or more.

The reaction temperature usually ranges about room temperature to 120° C.

The reaction time usually ranges about 30 minutes to 3 days.

(Step 3)

Compound [III-X-D1-903] can be obtained by a reaction of Compound [Z-X-D1-903-3] in a solvent under an acidic condition.

Examples of the acid for the acidic condition include hydrochloric acid, hydrobromic acid, sulfuric acid, and trifluoroacetic acid.

Examples of the solvent for the reaction include toluene, methanol, ethanol, isopropyl alcohol, tetrahydrofuran, dioxane, water; which may be used alone or as a mixture of two or more.

The reaction temperature usually ranges about room temperature to 120° C.

The reaction time usually ranges about 30 minutes to 3 days.

### Preparation Method 9

### Supplementary

As for a synthesis process of “ring P”, CAMILLE G. WERMUTH (1996) Molecular Variations Based on Isosteric Replacements. In CAMILLE G. WERMUTH (ed.) The Practice of Medicinal Chemistry, pp. 203-237. ACADEMIC PRESS can be used as a reference, besides the above Preparation methods 9-1, 9-2, 9-3.

### Preparation Method 9

### Outline

The following compound can be obtained by Preparation method 9.

TABLE 102

| | |
|---|---|
| | |
| [III-X-C-RWP] | [III-J-C-RWP] |
| | |
| [III-X-D2-RCP] | [III-J-D2-RCP] |
| | |
| [III-X-D1-RCP] | [III-J-D1-RCP] |

In the above table,

(that is, ring P) is

### Preparation Method 10

### Synthesis Process of “R^{a}”

### Preparation Method 10-1

Example of the synthesis process of “R^{a}”

(Step 1)

Compound [Z-X-RA-102] can be obtained by an amidation reaction of Compound [Z-X-RA-101] with piperidine in the presence of a condensation reagent in a solvent.

Examples of the solvent for the reaction include hydrocarbon solvent such as benzene, toluene, xylene, and hexane; halogenated solvent such as methylene chloride, chloroform, carbon tetrachloride, and 1,2-dichioroethane; ethers solvent such as diethyl ether, tetrahydrofuran, dioxane, 1,2-dimethoxyethane, and diglyme; polar solvent such as N,N-dimethylformamide, dimethyl sulfoxide, acetonitrile, and acetone; which may be used alone or as a mixture of two or more. Preferred examples of the solvent for the reaction include methylene chloride, chloroform, and N,N-dimethylformamide.

Examples of the condensation reagent for the reaction include water-soluble carbodiimide (WSC.HCl: 1-ethyl-3-(3-dimethylaminopropyl)carbodiimide hydrochloride), N,N′-dicyclohexylcarbodiimide (DCC), diphenylphosphoryl azide (DPPA), and carbonyldiimidazole (CDI). As necessary, 1-hydroxy-1H-benzotriazole monohydrate (HOBt.H₂O), 4-dimethylaminopyridine (DMAP), and the like may be added. Preferred examples of the condensation reagent include a mixture of water-soluble carbodiimide (WSC.HCl: 1-ethyl-3-(3-dimethylaminopropyl)carbodiimide hydrochloride) and 1-hydroxy-1H-benzotriazole monohydrate (HOBt.H₂O), or a mixture of water-soluble carbodiimide (WSC.HCl: 1-ethyl-3-(3-dimethylaminopropyl)carbodiimide hydrochloride) and 4-dimethylaminopyridine (DMAP).

The reaction temperature usually ranges about room temperature to 120° C., preferably from about room temperature to 100° C.

The reaction time usually ranges about 30 minutes to 3 days, preferably about 1 hr to 1 day.

(Step 2)

Compound [Z-X-RA-103] can be obtained by reacting Compound [Z-X-RA-102] with 1,1,3,3-tetramethyldisiloxane in the presence of (Ph₃P)IrCl(CO) in a solvent.

Examples of the solvent for the reaction include hydrocarbon solvent such as benzene, toluene, xylene, and hexane; halogenated solvent such as methylene chloride, chloroform, carbon tetrachloride, and 1,2-dichloroethane; ethers solvent such as diethyl ether, tetrahydrofuran, dioxane, 1,2-dimethoxyethane, and diglyme; polar solvent such as acetonitrile; which may be used alone or as a mixture of two or more. Preferred examples of the solvent for the reaction include toluene.

The reaction temperature usually ranges about 0° C. to 120° C., preferably from about room temperature to 100° C. The reaction time usually ranges about 30 minutes to 2 days, preferably about 30 minutes to 1 day.

(Step 3)

Compound [Z-X-RA-104] can be obtained by reacting Compound [Z-X-RA-103] with ethyl acrylate in a solvent.

Examples of the solvent for the reaction include hydrocarbon solvent such as benzene, toluene, xylene, and hexane; halogenated solvent such as methylene chloride, chloroform, carbon tetrachloride, and 1,2-dichloroethane; ethers solvent such as diethyl ether, tetrahydrofuran, dioxane, 1,2-dimethoxyethane, and diglyme; polar solvent such as N,N-dimethylformamide, dimethyl sulfoxide, acetonitrile, and acetone; which may be used alone or as a mixture of two or more. Preferred examples of the solvent for the reaction include acetonitrile.

The reaction temperature usually ranges about room temperature to 150° C., preferably from about room temperature to 120° C.

The reaction time usually ranges about 30 minutes to 2 days, preferably about 1 hr to 1 day.

(Step 4)

Compound [Z-X-RA-105] can be obtained by quaternizing the amino group of Compound [Z-X-RA-104] with p-toluenesulfonic acid and the like followed by reacting the quaternized Compound [Z-X-RA-104] with a base.

Examples of the base for the reaction include an aqueous solution of alkali metal hydroxide such as lithium hydroxide, sodium hydroxide, and potassium hydroxide. Preferred examples of the base for the reaction include an aqueous solution of potassium hydroxide.

The reaction temperature usually ranges about room temperature to 150° C., preferably from about room temperature to 120° C.

The reaction time usually ranges about 30 minutes to 2 days, preferably about 1 hr to 1 day.

(Step 5A)

3-Substituted cyclobutanecarboxylic acid [Z-X-RA-T-106] can be obtained by the catalytic hydrogenation reaction of Compound [Z-X-RA-105] in a solvent under normal pressure or medium pressure (for example, 3 atm).

Examples of the catalyst for the catalytic hydrogenation reaction include palladium on activated carbon, rhodium on activated carbon, palladium hydroxide, and Raney nickel. Preferred examples of the catalyst for the reaction include palladium on activated carbon and rhodium on activated carbon.

Examples of the solvent for the catalytic hydrogenation reaction include alcohols solvent such as methanol, ethanol, isopropyl alcohol, and tert-butanol; esters solvent such as ethyl acetate, methyl acetate, and butyl acetate; ethers solvent such as diethyl ether, tetrahydrofuran, dioxane, 1,2-dimethoxyethane, and diglyme; acetic acid, water; which may be used alone or as a mixture of two or more. Preferred examples of the solvent for the reaction include methanol, and tetrahydrofuran.

The reaction temperature usually ranges about room temperature to 100° C., preferably from about room temperature to 80° C.

The reaction time usually ranges about 30 minutes to 7 days, preferably about 1 hr to 5 days.

(Step 5B)

3-Substituted cyclobutanecarboxylic acid [Z-X-RA-C-106] can be obtained by a reduction reaction of Compound [Z-X-RA-105] using zinc in the presence of hydrochloric acid in a solvent.

[Z-X-RA-C-106] is a stereoisomer (cis-trans isomer) of [Z-X-RA-T-106].

Examples of the solvent for the reaction include ethers solvent such as diethyl ether, tetrahydrofuran, dioxane, 1,2-dimethoxyethane, and diglyme; acetic acid, water; which may be used alone or as a mixture of two or more. Preferred examples of the solvent for the reaction include tetrahydrofuran and water.

The reaction temperature usually ranges about room temperature to 150° C., preferably from about room temperature to 120° C.

The reaction time usually ranges about 30 minutes to 3 days, preferably about 1 hr to 1 day.

By Preparation method 5′, Preparation method 6, and the like, the following product (right column) wherein “R^{a}” is as shown in the following formulas can be obtained from [Z-X-RA-T-106] or [Z-X-RA-C-106] prepared in Preparation Method 10.

TABLE 103

| | Intermediate | Product |
|---|---|---|
| 10-1C | | |
| | [Z-X-RA-C-106] | [III-RA-C-X-B] |
| 10-1T | | |
| | [Z-X-RA-T-106] | [III-RA-T-X-B] |

In a similar way, the product (right column) can be obtained.

TABLE 104

| | Intermediate | Product |
|---|---|---|
| 10-2C | [Z-X-RA-C-106] | |
| | | [III-RA-C-X-C] |
| 10-2T | [Z-X-RA-T-106] | |
| | | [III-RA-T-X-C] |

TABLE 105

| | | |
|---|---|---|
| 10-3C | [Z-X-RA-C-106] | |
| | | [III-RA-C-X-D2] |
| 10-3T | [Z-X-RA-T-106] | |
| | | [III-RA-T-X-D2] |

TABLE 106

| | | |
|---|---|---|
| 10-4C | [Z-X-RA-C-106] | |
| | | [III-RA-C-X-D1] |
| 10-4T | [Z-X-RA-T-106] | |
| | | [III-RA-T-X-D1] |

For example, the following aspects are included.

TABLE 107

| | | |
|---|---|---|
| 10-5C | [Z-X-RA-C-106] | |
| | | [III-RA-C-F-D1] |

TABLE 108

| | | |
|---|---|---|
| 10-6C | [Z-X-RA-C-106] | |
| | | [III-RA-C-H-D1] |

TABLE 109

| | | |
|---|---|---|
| 10-7C | [Z-X-RA-C-106] | |
| | | [III-RA-C-K-D1] |

TABLE 110

| | | |
|---|---|---|
| 10-8C | [Z-X-RA-C-106] | |
| | | [III-RA-C-G-D1] |

TABLE 111

| | | |
|---|---|---|
| 10-9C | [Z-X-RA-C-106] | |
| | | [III-RA-C-L-D1] |

### Preparation Method 10-2

When R^{a} is “cross-linked cycloalkyl group which may optionally be substituted with the same or different 1 to 5 substituents selected from Group A”, for example, the following commercially available carboxylic acid compounds can be used.

For example, the following aspect is included.

When R^{a} is “spirocyclic cycloalkyl group which may optionally be substituted with the same or different 1 to 5 substituents selected from Group A”, for example, the following commercially available carboxylic acid compounds or ester compounds, or derivatives therefrom can be used.

When R^{a} is “a group which is substituted with amino group”; “a group which is substituted with alkylcarbonylamino group”; or “a group which is substituted with alkylsulfonylamino group”, for example, the following commercially available carboxylic acid compound, or derivatives therefrom can be used.

For example, the following aspect is included.

### EXAMPLES

According to the above preparation methods, the compounds listed in Tables 112 to 359 were prepared.

The following working Examples serve to illustrate the present invention more specifically, which does not intend to limit the present invention.

### Example E-58

### Sodium (S)-4-[4′-cyclopropyl-5-(2,2-dimethyl-propyl)-[3,5′]biisoxazolyl-3′-yl]-5-(2,4-dichloro-phenylcarbamoyl)-pentanoate

### E-58-1

### 5-Benzyloxy-pentanoic acid

Toluene (1700 mL) and aqueous 48 w/v % potassium hydroxide (942 mL) were mixed. δ-Valerolactone (300 g) was added dropwise to the mixture at 95° C. Benzyl bromide (1425 mL) was added dropwise to the reaction mixture at 115° C., and then the mixture was stirred for 3 hrs. Aqueous 48 w/v % potassium hydroxide (353 mL) was added to the reaction mixture at 115° C., and then the mixture was stirred for 3 hrs. Water (2100 mL) and tert-butyl methyl ether (420 mL) was added to the reaction mixture at ice temperature. The aqueous layer was separated, and washed with tert-butyl methyl ether (1500 mL×3). Toluene (1500 mL) and concentrated hydrochloric acid (420 mL) were added to the aqueous layer at ice temperature. The organic layer was separated, and washed with water (1500 mL×2). The organic layer was concentrated under reduced pressure to give the title compound (416.5 g, containing toluene 6 w/w %, by NMR) as a crude product.

### E-58-2

### (R)-4-Benzyl-3-(5-benzyloxy-pentanoyl)-oxazolidin-2-one

5-Benzyloxy-pentanoic acid (787 g, which is a corrected value regarding the amount of the contained solvent based on NMR) and acetonitrile (2200 mL) were mixed. (R)-4-Benzyl-2-oxazolidinone (559 g) and 4-dimethylaminopyridine (116 g) were added to the mixture. WSC.HCl (798 g) was added in two portions to the reaction mixture at room temperature. The resulting mixture was stirred overnight. Toluene (3500 mL) and aqueous 2 M hydrochloric acid (3000 mL) were added to the reaction mixture at room temperature. The organic layer was separated, and washed with aqueous 20 w/v % sodium chloride (2000 mL), a mixture of aqueous 7.5 w/v % sodium hydrogen carbonate (2000 mL) and aqueous 20 w/v % sodium chloride (1000 mL), and aqueous 20 w/v % sodium chloride (2000 mL) in this order. The organic layer was dried over sodium sulfate. The sodium sulfate was filtered off, and the filtrate was concentrated under reduced pressure to give the title compound (1164 g, containing toluene, by NMR) as a crude product.

### E-58-3

### tert-Butyl (S)-3-((R)-4-benzyl-2-oxo-oxazolidine-3-carbonyl)-6-benzyloxy-hexanoate

(R)-4-Benzyl-3-(5-benzyloxy-pentanoyl)-oxazolidin-2-one (1164 g, equivalent to 3.15 mol) and THF (2200 mL) were mixed. Sodium hexamethyldisilazide (1.9 M in THF) (2000 mL) was added dropwise to the mixture at −78° C. tert-Butyl bromoacetate (555 mL) was added dropwise to the reaction mixture at −78° C., and then the mixture was stirred for 3 hrs. N,N,N′-Trimethylethylenediamine (122 mL) was added dropwise to the reaction mixture. Aqueous 2 M hydrochloric acid (2810 mL) and THF (600 mL) were added to the reaction mixture at ice temperature. The organic layer was separated, and washed with water (2800 mL), aqueous 7.5 w/v % sodium hydrogen carbonate (2800 mL), and water (2800 mL) in this order. The organic layer was concentrated under reduced pressure to remove THF (about 1200 mL) partially. The resulting residue was directly used in the next step.

### E-58-4

### 4-tert-butyl (S)-2-(3-benzyloxy-propyl)-succinate

Lithium hydroxide monohydrate (145 g), THF (1120 mL), and water (1680 mL) were mixed. Aqueous 30 w/w % hydrogen peroxide (380 mL) was added dropwise to the mixture at ice temperature. The resulting mixture was stirred for 30 min tert-Butyl (S)-3-((R)-4-benzyl-2-oxo-oxazolidine-3-carbonyl)-6-benzyloxy-hexanoate (equivalent to 3.15 mol) was added dropwise to the reaction mixture at ice temperature with washing with THF (560 mL). The mixture was stirred for 1 hr at ice temperature, and for 2 hrs at room temperature. Aqueous 20 w/v % sodium hydrogen sulfite (3920 mL) was added dropwise to the reaction mixture at ice temperature. The organic layer was separated. Toluene (2800 mL) and water (1700 mL) were added to the organic layer. The resulting aqueous layer was separated and washed with toluene (2800 mL×2). Aqueous 2 M hydrochloric acid (1700 mL) was added to the combined aqueous layer, and the mixture was extracted with toluene (5000 mL). The organic layer was washed with water (2800 mL×2). The organic layer was concentrated under reduced pressure to give the title compound (870 g, containing toluene etc., by NMR) as a crude product.

### E-58-5

### 4-tert-butyl (S)-2-(3-benzyloxy-propyl)-succinate (R)-1-phenyl-ethylamine salt

4-tert-butyl (S)-2-(3-benzyloxy-propyl)-succinate (434 g, equivalent to 1.58 mol) and tert-butyl methyl ether (4200 mL) were mixed. (R)-1-Phenylethylamine (201 mL) was added dropwise to the mixture at room temperature. tert-Butyl methyl ether (840 mL) was added to the reaction mixture, and the resulting mixture was stirred for 1 hr at ice temperature. The resultant solid was collected by filtration, and dried under reduced pressure at room temperature. The solid (356 g) and tert-butyl methyl ether (2500 mL) were mixed, and the reaction temperature was risen to 85° C. The resulting mixture was stirred for 2 hrs at 55° C., and then overnight at room temperature. The resultant solid was collected by filtration, dried under reduced pressure at room temperature to give the title compound (321 g).

### E-58-6

### 4-tert-Butyl (S)-2-(3-benzyloxypropyl)-succinate

Aqueous 10 w/v % potassium hydrogen sulfate (1970 mL) and ethyl acetate (2180 mL) were mixed. 4-tert-butyl (S)-2-(3-benzyloxy-propyl)-succinate (R)-1-phenyl-ethylamine salt (544 g) was added in three portions to the mixture at room temperature. The resulting mixture was stirred for minutes at room temperature. The organic layer was separated, and washed with aqueous 10 w/v % potassium hydrogen sulfate, water, and aqueous 10 w/v % sodium chloride in this order. The organic layer was concentrated under reduced pressure, and then azeotroped with toluene to give the title compound (452 g, containing toluene) as a crude product. The resulting compound was analyzed using a chiral column. The retention time of the title compound was 13.1 min (the retention time of the optical isomer of the title compound was 12.1 min.), and the optical purity thereof was 99.9% ee.

The condition for the analysis using the chiral column was as follows:

Instrument: HPLC System Shimadzu High-performance liquid chromatograph Prominence

### Column: DAICEL CHIRALPAK AD-3R 0.46 cm φ×15 cm

Column temperature: 40° C.
Mobile phase:
(Solution A) 10 mM phosphate buffer (pH=2.6), (Solution B) acetonitrile
Composition of Mobile phase: Solution A:Solution B=55:45
Flow rate: 0.5 mL/min

### Detection: UV (220 nm)

### E-58-7

### tert-Butyl (S)-6-benzyloxy-3-(methoxy-methylcarbamoyl)-hexanoate

4-tert-Butyl (S)-2-(3-benzyloxypropyl)-succinate (322 g), N,O-dimethylhydroxylamine hydrochloride salt (117 g), HOBt.H₂O (30.6 g), and acetonitrile (480 mL) were mixed. Diisopropylethylamine (240 mL) was added to the mixture at ice temperature. WSC.HCl (230 g) was added in three portions to the reaction mixture at ice temperature, and the resulting mixture was stirred for 5 hrs at room temperature. Water was added to the reaction mixture. The resulting mixture was extracted with toluene. The organic layer was washed with aqueous 10 w/v % potassium hydrogen sulfate, aqueous 10 w/v % sodium chloride, aqueous 5 w/v % sodium hydrogen carbonate, and aqueous 10 w/v % sodium chloride in this order. The organic layer was concentrated under reduced pressure to give the title compound (500 g, containing toluene (13.4 w/w %), by NMR) as a crude product.

### E-58-8

### tert-Butyl (S)-6-benzyloxy-3-formyl-hexanoate

tert-Butyl (S)-6-benzyloxy-3-(methoxy-methylcarbamoyl)-hexanoate (115 g, equivalent to 273 mmol) and THF (500 mL) were mixed. Diisobutylaluminium hydride (1.0 M in toluene) (356 mL) was added dropwise to the mixture at −78° C. The reaction mixture was stirred for 2 hrs at −78° C., and then was added dropwise to aqueous 1.0 M sulfuric acid (500 mL) at ice temperature. Ethyl acetate (500 mL) was added to the mixture. The organic layer was separated, and washed with 0.5 M sulfuric acid (500 mL×2), water (500 mL), and brine (500 mL) in this order. The organic layer was dried over sodium sulfate. The sodium sulfate was filtered off, and the organic layer was concentrated under reduced pressure to give the title compound (71.4 g) as a crude product.

### E-58-9

### tert-Butyl (S)-6-benzyloxy-3-(hydroxyimino-methyl)-hexanoate

Hydroxylammonium chloride (11.6 g), ethanol (150 mL), and water (450 mL) were mixed. Aqueous 4 M sodium hydroxide (41.8 mL) was added dropwise to the mixture at ice temperature. A solution of tert-butyl (S)-6-benzyloxy-3-formyl-hexanoate (46.5 g, equivalent to 139 mmol) in ethanol (25 mL) and THF (50 m) was added dropwise to the reaction mixture at ice temperature. The mixture was stirred for 1 hr at ice temperature, and then toluene (300 mL) was added thereto. The organic layer was separated, and washed with water (300 mL) and brine (150 mL) in this order. The organic layer was dried over sodium sulfate. The sodium sulfate was filtered off, and the filtrate was concentrated under reduced pressure to give the title compound (47.7 g) as a crude product.

### E-58-10

### tert-Butyl (S)-6-benzyloxy-3-(chloro-hydroxyimino-methyl)-hexanoate

tert-Butyl (S)-6-benzyloxy-3-(hydroxyimino-methyl)hexanoate (15 g) and DMF (38 mL) were mixed. A solution of N-chlorosuccinimide (6.55 g) in DMF (23 mL) was added dropwise to the mixture under water-cooling. The resulting mixture was stirred for 1 hr at room temperature, and then toluene (90 mL) and water (90 mL) were added to the reaction mixture. The organic layer was separated, and washed with water (45 mL) and brine (45 mL) in this order, and the solution was directly used in the next step.

### E-58-11

### tert-Butyl (S)-6-benzyloxy-3-(5-hydroxymethyl-isoxazol-3-yl)-hexanoate

Propargyl alcohol (37 g), toluene (40 mL), and aqueous 2 M potassium carbonate (25 mL) were mixed. The mixture was heated to 110° C. A solution of tert-butyl (S)-6-benzyloxy-3-(chloro-hydroxyimino-methyl)-hexanoate which was obtained in E-58-10 in toluene (equivalent to 46.7 mmol) was added dropwise to the reaction mixture, and the resulting mixture was stirred for 1 hr. Water was added to the reaction mixture. The organic layer was separated, and washed with water, and concentrated under reduced pressure. The resulting residue was purified by silica-gel column chromatography (Eluent: ethyl acetate/hexane=1/1) to give the title compound (14.6 g).

### E-58-12

### tert-Butyl (S)-6-benzyloxy-3-(5-hydroxymethyl-4-iodo-isoxazol-3-yl)-hexanoate

tert-Butyl (S)-6-benzyloxy-3-(5-hydroxymethyl-isoxazol-3-yl)-hexanoate (17.2 g) and acetonitrile (200 mL) were mixed. N-Iodosuccinimide (20.7 g) and ammonium cerium(IV) nitrate (12.6 g) were added to the mixture under water-cooling. The reaction mixture was stirred for 13 hrs at room temperature, and then added dropwise to aqueous 10 w/v % sodium sulfite (187 mL). To the mixture was added acetonitrile. And the mixture was filtered using Celite. The filtrate was concentrated under reduced pressure, and the resulting residue was purified by silica-gel column chromatography (Eluent: ethyl acetate/hexane=1/2) to give the title compound (9.25 g).

### E-58-13

### tert-Butyl (S)-6-benzyloxy-3-(5-formyl-4-iodo-isoxazol-3-yl)-hexanoate

tert-Butyl (S)-6-benzyloxy-3-(5-hydroxymethyl-4-iodo-isoxazol-3-yl)-hexanoate (9.25 g) and dichloromethane (70 mL) were mixed. Dess-Martin periodinane (8.77 g) was added to the mixture at ice temperature. The resulting mixture was stirred for 30 minutes at room temperature. Aqueous 20 w/v % sodium sulfite was added to the reaction mixture at ice temperature. Diethyl ether and aqueous saturated sodium hydrogen carbonate were added to the mixture. The resulting mixture was stirred for 1 hr at room temperature. The organic layer was separated, and washed with aqueous saturated sodium hydrogen carbonate (three times) and brine in this order. The organic layer was dried over magnesium sulfate. The magnesium sulfate was filtered off, and the filtrate was concentrated under reduced pressure to give the title compound (9.82 g).

### E-58-14

### tert-Butyl (S)-6-benzyloxy-3-[5-(hydroxyimino-methyl)-4-iodo-isoxazol-3-yl]-hexanoate

Hydroxylammonium chloride (1.79 g), THF (20 mL), and water (15 mL) were mixed. Aqueous 4 M sodium hydroxide (6.45 mL) was added dropwise to the mixture at ice temperature. A mixture of tert-butyl (S)-6-benzyloxy-3-(5-formyl-4-iodo-isoxazol-3-yl)-hexanoate (9.8 g) and ethanol (50 mL) was added dropwise to the reaction mixture at ice temperature. The mixture was stirred at room temperature overnight. Then the pH of the reaction mixture was adjusted with aqueous 2 M hydrochloric acid to 4. Ethyl acetate was added to the reaction mixture. The organic layer was separated, and washed with water and brine in this order, and then concentrated under reduced pressure to give the title compound (9.48 g) as a crude product.

### E-58-15

### tert-Butyl (S)-6-benzyloxy-3-[5-(chloro-hydroxyimino-methyl)-4-iodo-isoxazol-3-yl]-hexanoate

tert-Butyl (S)-6-benzyloxy-3-[5-(hydroxyimino-methyl)-4-iodo-isoxazol-3-yl]-hexanoate (2.5 g, equivalent to 4.38 mmol) and DMF (9.0 mL) were mixed. N-Chlorosuccinimide (646 mg) was added to the mixture under water-cooling. The resulting mixture was stirred for 1 hr at room temperature. Toluene and water were added to the reaction mixture. The organic layer was separated, and washed with water (twice) and brine in this order, and the solution was directly used in the next step.

### E-58-16

### 1,1-Dibromo-4,4-dimethyl-1-pentene

A solution of triphenylphosphine (29.2 g) in dichloromethane (60 mL) was added dropwise to a solution of carbon tetrabromide (18.5 g) in dichloromethane (60 mL) at ice temperature. The mixture was stirred at ice temperature for 10 min. A solution of 3,3-dimethyl-butylaldehyde (3.99 g) in dichloromethane (10 mL) was added dropwise to the reaction mixture at ice temperature. The mixture was stirred for 2 hrs at ice temperature. The resultant precipitate was filtered off with washing with hexane. The filtrate was concentrated under reduced pressure. The resulting residue was purified by silica-gel column chromatography (Eluent: hexane) to give the title compound (7.85 g).

### E-58-17

### 4,4-Dimethyl-1-pentyne

1,1-Dibromo-4,4-dimethyl-1-pentene (7.8 g), THF (16 mL), and hexane (16 mL) were mixed. n-Butyllithium (2.69 M in hexane) (23 mL) was added dropwise to the mixture at −78° C. The reaction mixture was stirred for 1 hr at −78° C., and then acetic acid (0.88 mL) was added to the reaction mixture. Hexane and aqueous saturated ammonium chloride was added thereto at ice temperature. The organic layer was separated, and dried over sodium sulfate. The sodium sulfate was filtered off. The title compound was obtained as a solution in THF-hexane (approximately 0.165 M) without purification.

### E-58-18

### tert-Butyl (S)-6-benzyloxy-3-[5-(2,2-dimethylpropyl)-4′-iodo-[3,5′]biisoxazolyl-3′-yl]-hexanoate

A solution of the resulting 4,4-dimethyl-1-pentyne in THF-hexane which is obtained in E-58-17 (70 mL, equivalent to 11.5 mmol), potassium carbonate (666 mg), and water (4.0 mL) were mixed. The mixture was heated to 100° C. A solution of tert-butyl (S)-6-benzyloxy-3-[5-(chloro-hydroxyimino-methyl)-4-iodo-isoxazol-3-yl]-hexanoate which is obtained in E-58-15 in toluene (equivalent to 4.38 mmol) was added dropwise to the mixture. The resulting mixture was stirred for 20 min. Ethyl acetate was added to the reaction mixture. The organic layer was separated, and washed with aqueous saturated ammonium chloride (twice) and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/10) to give the title compound (2.00 g, containing ethyl acetate (5.8 w/w %), by NMR).

### E-58-19

### tert-Butyl (S)-6-benzyloxy-3-[4′-cyclopropyl-5-(2,2-dimethyl-propyl)-[3,5′]biisoxazolyl-3′-yl]-hexanoate

tert-Butyl (S)-6-benzyloxy-3-[5-(2,2-dimethyl-propyl)-4′-iodo-[3,5′]biisoxazolyl-3′-yl]-hexanoate (1.89 g), 2-cyclopropyl-4,4,5,5-tetramethyl-[1,3,2]dioxaborolane (1.04 g), tripotassium phosphate (2.63 g), PdCl₂(PPh₃)₂ (218 mg), and DMF (18 mL) were mixed. The reaction mixture was degassed by bubbling argon, and the resulting mixture was stirred at 90° C. overnight. Water was added to the reaction mixture, and the resulting mixture was filtered, and to the filtrate was added ethyl acetate. The organic layer was separated, and washed with aqueous 1 M hydrochloric acid, water (twice), and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/13) to give the title compound (1.69 g).

### E-58-20

### (S)-6-Benzyloxy-3-[4′-cyclopropyl-5-(2,2-dimethyl-propyl)-[3,5′]biisoxazolyl-3′-yl]-hexanoic acid

tert-Butyl (S)-6-benzyloxy-3-[4′-cyclopropyl-5-(2,2-dimethyl-propyl)-[3,5′]biisoxazolyl-3′yl]-hexanoate (1.68 g) and toluene (15 mL) were mixed. Trifluoroacetic acid (6.5 mL) was added to the mixture at ice temperature. The mixture was stirred at room temperature overnight. The reaction mixture was concentrated under reduced pressure. The resulting residue was azeotroped twice with toluene to give the title compound (1.60 g, containing toluene (18 w/w %), by NMR) as a crude product.

### E-58-21

### (S)-6-Benzyloxy-3-[4′-cyclopropyl-5-(2,2-dimethyl-propyl)-[3,5′]biisoxazolyl-3′-yl]-hexanoic acid (2,4-dichloro-phenyl)-amide

(S)-6-Benzyloxy-3-[4′-cyclopropyl-5-(2,2-dimethyl-propyl)-[3,5′]biisoxazolyl-3′-yl]-hexanoic acid (300 mg, equivalent to 0.53 mmol) and dimethylacetamide (2.5 mL) were mixed. Thionyl chloride (0.051 mL) was added to the mixture at ice temperature. 2,4-Dichloro-phenylamine (104 mg) was added to the reaction mixture at ice temperature. The cooling bath was removed, and the reaction mixture was stirred for 1 hr. Diethyl ether and water was added to the reaction mixture. The organic layer was separated, and washed with aqueous saturated sodium bicarbonate and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/5) to give the title compound (184 mg).

### E-58-22

### (S)-3-[4′-Cyclopropyl-5-(2,2-dimethyl-propyl)-[3,5′]biisoxazolyl-3′-yl]-6-hydroxy-hexanoic acid (2,4-dichloro-phenyl)-amide

(S)-6-Benzyloxy-3-[4′-cyclopropyl-5-(2,2-dimethyl-propyl)-[3,5′]biisoxazolyl-3′-yl]-hexanoic acid (2,4-dichloro-phenyl)-amide (264 mg) and dichloromethane (4.0 mL) were mixed. Boron tribromide (1 M in dichloromethane) (1.1 mL) was added to the mixture at ice temperature. The cooling bath was removed, and the reaction mixture was stirred for 30 minutes. Aqueous saturated sodium hydrogen carbonate and diethyl ether were added to the reaction mixture at ice temperature. The organic layer was separated, and washed with brine. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/1) to give the title compound (183 mg).

### E-58-23

### (S)-4-[4′-Cyclopropyl-5-(2,2-dimethyl-propyl)-[3,5′]biisoxazolyl-3′-yl]-5-(2,4-dichloro-phenylcarbamoyl)-pentanoic acid

(S)-3-[4′-cyclopropyl-5-(2,2-dimethyl-propyl)-[3,5′]biisoxazolyl-3′-yl]-6-hydroxy-hexanoic acid (2,4-dichloro-phenyl)-amide (180 mg), acetonitrile (1.3 mL), and 1.0 M phosphate buffer (pH=6.8) (1.3 mL) were mixed. 2,2,6,6-Tetramethyl-1-piperidinyloxy radical (TEMPO) (5.0 mg) and sodium chlorite (78 mg) were added to the mixture at room temperature. Aqueous sodium hypochlorite (Wako Pure Chemical Industries, 0.090 mL) was added dropwise to the reaction mixture at ice temperature. The resulting mixture was stirred for 1 hr at room temperature. Aqueous 20 w/v % sodium sulfite and ethyl acetate were added to the reaction mixture at ice temperature. The organic layer was separated, and washed with brine, and then dried over sodium sulfate. The sodium sulfate was filtered off, and the filtrate was concentrated under reduced pressure. The resulting residue was purified by silica-gel column chromatography (Eluent: ethyl acetate/hexane=1/1), and then azeotroped twice with ethanol to give the title compound (237 mg, containing ethanol (31.9 w/w %), by NMR).

¹H-NMR (400 MHz, DMSO-d6) 0.42-0.54 (m, 1H), 0.59-0.71 (m, 1H), 0.84-1.02 (m, 2H), 0.95 (s, 9H), 1.67-1.77 (m, 1H), 1.90-2.05 (m, 2H), 2.15-2.33 (m, 2H), 2.76 (s, 2H), 2.85 (dd, J=15.20, 6.40 Hz, 1H), 2.95 (dd, J=15.20, 8.40 Hz, 1H), 3.52-3.65 (m, 1H), 6.74 (s, 1H), 7.36 (dd, J=8.80, 2.00 Hz, 1H), 7.60 (d, J=2.00 Hz, 1H), 7.63 (d, J=8.80 Hz, 1H), 9.68 (s, 1H)

### E-58-24

### Sodium (S)-4-[4′-cyclopropyl-5-(2,2-dimethyl-propyl)-[3,5′]biisoxazolyl-3′-yl]-5-(2,4-dichloro-phenylcarbamoyl)-pentanoate

(S)-4-[4′-Cyclopropyl-5-(2,2-dimethyl-propyl)-[3,5′]biisoxazolyl-3′-yl]-5-(2,4-dichloro-phenylcarbamoyl)-pentanoic acid (237 mg, equivalent to 0.300 mmol) and ethanol (4.0 mL) were mixed. Aqueous 1 M sodium hydroxide (0.300 mL) was added to the mixture at room temperature. The resulting solution was concentrated under reduced pressure to give the title compound (149 mg).

### Example E-49

### Sodium (S)-5-(4-chloro-2-fluoro-phenylcarbamoyl)-4-[4′-cyclopropyl-5-(1,1-difluoro-2,2-dimethylpropyl)-[3,5′]biisoxazolyl-3′-yl]-pentanoate

### E-49-1

### tert-Butyl (S)-6-benzyloxy-3-(4-cyclopropyl-5-hydroxymethyl-isoxazol-3-yl)-hexanoate

tert-Butyl (S)-6-benzyloxy-3-(5-hydroxymethyl-4-iodo-isoxazol-3-yl)-hexanoate (17.2 g) which obtained by a similar reaction to that described in E-58-12, 2-cyclopropyl-4,4,5,5-tetramethyl-[1,3,2]dioxaborolane (11.5 g), tripotassium phosphate (29.1 g), PdCl₂(PPh₃)₂ (2.4 mg), and DMF (130 mL) were mixed. The reaction mixture was degassed by bubbling argon, and the resulting mixture was stirred at 90° C. overnight. Water (150 mL) was added to the reaction mixture. The resulting mixture was filtered. To the filtrate was added ethyl acetate. The organic layer was separated, and washed with aqueous 2 M hydrochloric acid, water (twice), and brine in this order. Then, the organic layer was dried over sodium sulfate. The sodium sulfate was filtered off, and the filtrate was concentrated under reduced pressure. The resulting residue was purified by silica-gel column chromatography (Eluent: ethyl acetate/hexane=1/2) to give the title compound (5.24 g).

### E-49-2

### tert-Butyl (S)-6-benzyloxy-3-(4-cyclopropyl-5-formyl-isoxazol-3-yl)-hexanoate

tert-Butyl (S)-6-benzyloxy-3-(4-cyclopropyl-5-hydroxymethyl-isoxazol-3-yl)-hexanoate (5.2 g) and chloroform (42 mL) were mixed. Dess-Martin periodinane (6.6 g) was added to the mixture at ice temperature. The resulting mixture was stirred for 1 hr at room temperature. Aqueous 20 w/v % sodium sulfite was added to the reaction mixture at ice temperature. Diethyl ether and aqueous saturated sodium hydrogen carbonate were added to the mixture, and the resulting mixture was stirred for 1 hr at room temperature. The organic layer was separated, and washed with aqueous saturated sodium hydrogen carbonate (twice) and brine in this order. The organic layer was dried over sodium sulfate. The sodium sulfate was filtered off, and the filtrate was concentrated under reduced pressure to give the title compound (4.87 g) as a crude product.

### E-49-3

### tert-Butyl (S)-6-benzyloxy-3-[4-cyclopropyl-5-(hydroxyimino-methyl)-isoxazol-3-yl]-hexanoate

Hydroxylammonium chloride (1.32 g), THF (9.8 mL), and water (7.3 mL) were mixed. Aqueous 4 M sodium hydroxide (4.73 mL) was added dropwise to the mixture at ice temperature. A mixture of tert-butyl (S)-6-benzyloxy-3-(4-cyclopropyl-5-formyl-isoxazol-3-yl)-hexanoate (4.87 g) and ethanol (24.5 mL) was added dropwise to the reaction mixture at ice temperature. The resulting mixture was stirred at room temperature overnight. Then the pH of the reaction mixture was adjusted with aqueous 2 M hydrochloric acid to 3. Ethyl acetate was added to the mixture. The organic layer was separated, and washed with water (twice) and brine in this order, and then concentrated under reduced pressure to give the title compound (4.87 g).

### E-49-4

### tert-Butyl (S)-6-benzyloxy-3-[4-cyclopropyl-5-(chloro-hydroxyimino-methyl)-isoxazol-3-yl]-hexanoate

tert-Butyl (S)-6-benzyloxy-3-[4-cyclopropyl-5-(hydroxyimino-methyl)-isoxazol-3-yl]-hexanoate (2.00 g, equivalent to 4.39 mmol) and DMF (8.0 mL) were mixed. N-chlorosuccinimide (647 mg) was added to the mixture at room temperature. The reaction mixture was stirred for 1 hr at room temperature, and then N-chlorosuccinimide (647 mg) was additionally added to the reaction mixture. The resulting mixture was stirred for 1 hr at room temperature, and then toluene and water were added to the reaction mixture. The organic layer was separated, and washed with water (three times) and brine in this order. The solution was used in the next step without purification.

### E-49-5

### 4,4-Dimethyl-1-pentyn-3-ol

Ethynyltrimethylsilane (5.40 g) and THF (50 mL) were mixed. n-Butyllithium (2.69 M in hexane) (19.5 mL) was added dropwise to the mixture at −78° C. The reaction mixture was stirred for 30 min at −78° C. 2,2-Dimethylpropionaldehyde (5.52 mL) was added to the mixture, and the resulting mixture was stirred for 1 hr at room temperature. Diethyl ether and water were added to the mixture at ice temperature. The organic layer was separated, and washed with brine, and then dried over sodium sulfate. The sodium sulfate was filtered off, and the filtrate was concentrated under reduced pressure. Methanol (120 mL) and potassium carbonate (20.73 g) were added to the residue at room temperature. The resulting mixture was stirred for 30 minutes. The solid was filtered off, and the filtrate was concentrated under reduced pressure until reduced by approximately half in volume. Dichloromethane and aqueous saturated ammonium chloride were added to the mixture. The organic layer was separated, and dried over sodium sulfate. The sodium sulfate was filtered off, and the filtrate was concentrated under reduced pressure to give the title compound (5.77 g, containing solvents (29 w/w %) including dichloromethane) as a crude product.

### E-49-6

### tert-Butyl (S)-6-benzyloxy-3-[4′-cyclopropyl-5-(1-hydroxy-2,2-dimethyl-propyl)-[3,5′]biisoxazolyl-3′-yl]-hexanoate

4,4-Dimethyl-1-pentyn-3-ol which was obtained in E-49-(1.73 g, equivalent to 11.0 mmol), aqueous 1.0 M potassium carbonate (4.67 mL), and toluene (5.0 mL) were mixed, and the mixture was heated to 100° C. A solution of tert-butyl (S)-6-benzyloxy-3-[4-cyclopropyl-5-(chloro-hydroxyimino-methyl)-isoxazol-3-yl]-hexanoate which was obtained in E-49-4 (equivalent to 4.39 mmol) in toluene was added dropwise to the reaction mixture, and the resulting mixture was stirred for 40 min. Ethyl acetate was added to the reaction mixture. The organic layer was separated, and washed with aqueous saturated ammonium chloride (twice) and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/4) to give the title compound (2.62 g, containing ethyl acetate (6.1 w/w %), by NMR).

### E-49-7

### tert-Butyl (S)-6-benzyloxy-3-[4′-cyclopropyl-5-(2,2-dimethyl-propionyl)-[3,5′]biisoxazolyl-3′-yl]-hexanoate

tert-Butyl (S)-6-benzyloxy-3-[4′-cyclopropyl-5-(1-hydroxy-2,2-dimethyl-propyl)-[3,5′]biisoxazolyl-3′-yl]-hexanoate (2.45 g), chloroform (25 mL) were mixed. Dess-Martin periodinane (2.36 g) was added to the mixture at ice temperature. The reaction mixture was stirred for 1 hr at room temperature, and to the mixture was added aqueous 20 w/v % sodium sulfite at ice temperature. To the reaction mixture were added ethyl acetate and aqueous saturated sodium hydrogen carbonate. The resulting mixture was stirred for 30 minutes at room temperature. The organic layer was separated, and washed with aqueous saturated sodium hydrogen carbonate (twice) and brine in this order. The organic layer was dried over sodium sulfate. The sodium sulfate was filtered off, and the filtrate was concentrated under reduced pressure to give the title compound (2.48 g) as a crude product.

### E-49-8

### tert-Butyl (S)-6-benzyloxy-3-[4′-cyclopropyl-5-(1,1-difluoro-2,2-dimethyl-propyl)-[3,5′]biisoxazolyl-3′-yl]-hexanoate

tert-Butyl (S)-6-benzyloxy-3-[4′-cyclopropyl-5-(2,2-dimethyl-propionyl)-[3,5′]biisoxazolyl-3′-yl]-hexanoate (2.48 g) and bis(2-methoxyethyl)aminosulfur trifluoride (15 mL) were mixed. The mixture was stirred for 1 hr at 80° C. Chloroform (15 mL) was added to the reaction mixture at ice temperature. The reaction mixture was poured over ice. Aqueous saturated sodium hydrogen carbonate was added dropwise to the mixture at ice temperature. Ethyl acetate was added to the mixture. The organic layer was separated, and washed with aqueous saturated sodium hydrogen carbonate and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/10) to give the title compound (2.38 g).

### E-49-9

### (S)-6-Benzyloxy-3-[4′-cyclopropyl-5-(1,1-difluoro-2,2-dimethyl-propyl)-[3,5′]biisoxazolyl-3′-yl]-hexanoic acid

tert-Butyl (S)-6-benzyloxy-3-[4′-cyclopropyl-5-(1,1-difluoro-2,2-dimethyl-propyl)-[3,5′]biisoxazolyl-3′-yl]-hexanoate (1.68 g) and toluene (20 mL) were mixed. To the mixture was added trifluoroacetic acid (5.0 mL) at ice temperature. The reaction mixture was stirred at room temperature overnight, and concentrated under reduced pressure. The resulting residue was azeotroped twice with toluene to give the title compound (2.28 g, containing toluene (15.7 w/w %), by NMR) as a crude product.

### E-49-10

### (S)-6-Benzyloxy-3-[4′-cyclopropyl-5-(1,1-difluoro-2,2-dimethylpropyl)-[3,5′]biisoxazolyl-3′-yl]-hexanoic acid (4-chloro-2-fluoro-phenyl)-amide

(S)-6-Benzyloxy-3-[4′-cyclopropyl-5-(1,1-difluoro-2,2-dimethyl-propyl)-[3,5′]biisoxazolyl-3′-yl]-hexanoic acid (356 mg, equivalent to 0.597 mmol), 4-chloro-2-fluoro-phenylamine (0.079 mL), and DMF (3.0 mL) were mixed. To the mixture were added diisopropylethylamine (0.144 mL) and HATU (273 mg) at ice temperature. The resulting mixture was stirred at room temperature overnight. To the reaction mixture was added aqueous saturated sodium bicarbonate at ice temperature. The mixture was extracted with ethyl acetate. The organic layer was washed with water (twice) and brine in this order. The organic layer was dried over sodium sulfate. The sodium sulfate was filtered off, and the filtrate was concentrated under reduced pressure. The resulting residue was purified by preparative chromatography (Eluent: ethyl acetate/hexane=1/3) to give the title compound (322 mg).

### E-49-11

### (S)-3-[4′-Cyclopropyl-5-(1,1-difluoro-2,2-dimethyl-propyl)-[3,5′]biisoxazolyl-3′-yl]-6-hydroxy-hexanoic acid (4-chloro-2-fluoro-phenyl)-amide

(S)-6-Benzyloxy-3-[4′-cyclopropyl-5-(1,1-difluoro-2,2-dimethyl-propyl)-[3,5′]biisoxazolyl-3′-yl]-hexanoic acid (4-chloro-2-fluoro-phenyl)-amide (322 mg), dichloromethane (3.2 mL) were mixed. To the mixture was added boron tribromide (1 M in dichloromethane) (1.5 mL) at ice temperature. The cooling bath was removed, and the reaction mixture was stirred for 1 hr. To the reaction mixture was added aqueous saturated sodium hydrogen carbonate. To the reaction mixture was added diethyl ether. The organic layer was separated, and washed with brine. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/2) to give the title compound (175 mg).

### E-49-12

### (S)-5-(4-Chloro-2-fluoro-phenylcarbamoyl)-4-[4′-cyclopropyl-5-(1,1-difluoro-2,2-dimethyl-propyl)-[3,5′]biisoxazolyl-3′-yl]-pentanoic acid

(S)-3-[4′-Cyclopropyl-5-(1,1-difluoro-2,2-dimethyl-propyl)-[3,5′]biisoxazolyl-3′-yl]-6-hydroxy-hexanoic acid (4-chloro-2-fluoro-phenyl)-amide (175 mg), acetonitrile (1.3 mL), and 1.0 M phosphate buffer (pH=6.8) (1.3 mL) were mixed. To the mixture were added 2,2,6,6-tetramethyl-1-piperidinyloxy radical (TEMPO) (5.0 mg) and sodium chlorite (73 mg) at room temperature. Aqueous sodium hypochlorite (Wako Pure Chemical Industries, 0.088 mL) was added dropwise to the reaction mixture at room temperature. The reaction mixture was stirred for 1 hr at room temperature. Aqueous 20 w/v % sodium sulfite was added to the reaction mixture at ice temperature. Ethyl acetate was added to the mixture. The organic layer was separated, and washed with brine, and then dried over sodium sulfate. The sodium sulfate was filtered off, and the filtrate was concentrated under reduced pressure. The resulting residue was purified by silica-gel column chromatography (Eluent: ethyl acetate/hexane=1/2), and then azeotroped twice with ethanol to give the title compound (177 mg, containing ethanol (5.1 w/w %), by NMR).

¹H-NMR (400 MHz, DMSO-d6) 0.45-0.57 (m, 1H), 0.64-0.75 (m, 1H), 0.89-1.02 (m, 2H), 1.08 (s, 9H), 1.72-1.82 (m, 1H), 1.89-2.04 (m, 2H), 2.17-2.32 (m, 2H), 2.87 (dd, J=15.60, 6.40 Hz, 1H), 2.97 (dd, J=15.60, 8.80 Hz, 1H), 3.55-3.66 (m, 1H), 7.17-7.23 (m, 1H), 7.39 (s, 1H), 7.43 (dd, J=10.40, 2.00 Hz, 1H), 7.83 (t, J=8.80 Hz, 1H), 9.92 (s, 1H), 12.16 (brs, 1H)

### E-49-13

### Sodium (S)-5-(4-chloro-2-fluoro-phenylcarbamoyl)-4-[4′-cyclopropyl-5-(1,1-difluoro-2,2-dimethylpropyl)-[3,5′]biisoxazolyl-3′-yl]-pentanoate

(S)-5-(4-Chloro-2-fluoro-phenylcarbamoyl)-4-[4′-cyclopropyl-5-(1,1-difluoro-2,2-dimethylpropyl)-[3,5′]biisoxazolyl-3′-yl]-pentanoic acid (177 mg, equivalent to 0.303 mmol) and ethanol (1.5 mL) were mixed. To the mixture was added aqueous 1 M sodium hydroxide (0.303 mL) at room temperature. The resulting solution was concentrated under reduced pressure to give the title compound (152 mg).

### Example E-52

### Sodium (S)-5-(2-chloro-4-methyl-phenylcarbamoyl)-4-[4-cyclopropyl-5′-(1,1-difluoro-2,2-dimethylpropyl)-[3,3′]biisoxazolyl-5′-yl]pentanoate

### E-52-1

### tert-Butyl (S)-6-benzyloxy-3-ethynyl-hexanoate

Trimethylsilyldiazomethane (2 M in hexane) (47 mL) and THF (125 mL) were mixed. n-Butyllithium (2.6 M in hexane) (72.1 mL) was added dropwise to the mixture at −78° C. The reaction mixture was stirred for 20 min at −78° C. A solution of tert-butyl (S)-6-benzyloxy-3-formyl-hexanoate which is obtained in a similar way to E-58-8 (25 g) in THF (75 mL) was added dropwise to the reaction mixture. The reaction mixture was stirred for 20 min at −78° C., and then for 2 hrs at 0° C. To the reaction mixture was added aqueous saturated ammonium chloride at ice temperature. Ethyl acetate was added to the mixture. The organic layer was separated, and washed with water and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/8) to give the title compound (12.9 g, containing toluene (14.9 w/w %), by NMR).

### E-52-2

### (tert-Butyl-dimethyl-silanyloxy)-acetaldehyde oxime

Hydroxylammonium chloride (1.62 g), THF (5.5 mL), and water (4.0 mL) were mixed. Aqueous 4 M sodium hydroxide (5.81 mL) was added dropwise thereto at ice temperature. A mixture of (tert-butyl-dimethylsilanyloxy)-acetaldehyde (2.7 g) and ethanol (13 mL) was added dropwise to the reaction mixture at ice temperature. The reaction mixture was stirred at room temperature overnight. Then the pH of the reaction mixture was adjusted with aqueous 2 M hydrochloric acid to 4. Ethyl acetate was added to the mixture. The organic layer was separated, and washed with water (twice) and brine in this order, and then concentrated under reduced pressure to give the title compound (2.49 g).

### E-52-3

### (tert-Butyl-dimethylsilanyloxy)-1-chloro-acetaldehyde oxime

tert-Butyl-dimethylsilanyloxy-acetaldehyde oxime (2.4 g) and DMF (7.0 mL) were mixed. A solution of N-chlorosuccinimide (2.04 g) in DMF (5.0 mL) was added to the mixture at room temperature. The reaction mixture was stirred for 30 minutes at room temperature. To the reaction mixture were added toluene and water. The organic layer was separated, and washed with water (three times) and brine in this order, and the solution was directly used in the next step.

### E-52-4

### tert-Butyl (S)-6-benzyloxy-3-[3-(tert-butyl-dimethylsilanyloxymethyl)-isoxazol-5-yl]hexanoate

tert-Butyl (S)-6-benzyloxy-3-ethynylhexanoate which was obtained in E-52-1 (3.84 g), toluene (1.5 mL), potassium carbonate (139 mg), and water (1.0 mL) were mixed, and the mixture was heated to 100° C. A solution of (tert-butyl-dimethylsilanyloxy)-1-chloro-acetaldehyde oxime which was obtained in E-52-3 in toluene (equivalent to 12.7 mmol) was added dropwise to the reaction mixture, and the resulting mixture was stirred for 30 minutes. Ethyl acetate was added to the reaction mixture. The organic layer was separated, and washed with water and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/12) to give the title compound (2.78 g, containing ethyl acetate (5.6 w/w %), by NMR).

### E-52-5

### tert-Butyl (S)-6-benzyloxy-3-(3-hydroxymethyl-isoxazol-5-yl)hexanoate

tert-Butyl (S)-6-benzyloxy-3-[3-(tert-butyl-dimethylsilanyloxymethyl)-isoxazol-5-yl]hexanoate (2.63 g) and THF (11 mL) were mixed. To the mixture was added tetrabutylammonium fluoride (1 M in THF) (6.44 mL) at ice temperature. The mixture was stirred for 2 hrs at room temperature. To the reaction mixture were added ethyl acetate and aqueous saturated ammonium chloride. The organic layer was separated, and washed with brine, and then dried over sodium sulfate. The sodium sulfate was filtered off, and the filtrate was concentrated under reduced pressure. The resulting residue was purified by silica-gel column chromatography (Eluent: ethyl acetate/hexane=1/1) to give the title compound (2.04 g, containing ethyl acetate (8.0 w/w %), by NMR).

### E-52-6

### tert-Butyl (S)-6-benzyloxy-3-(3-hydroxymethyl-4-iodo-isoxazol-5-yl)hexanoate

tert-Butyl (S)-6-benzyloxy-3-(3-hydroxymethyl-isoxazol-5-yl)hexanoate (2.0 g, equivalent to 5.36 mmol) and acetonitrile (16 mL) were mixed. To the mixture were added N-iodosuccinimide (2.4 g) and ammonium cerium(IV) nitrate (1.18 g) at room temperature. The reaction mixture was stirred overnight at room temperature. Aqueous 20 w/v % sodium sulfite was added dropwise to the reaction mixture at ice temperature. Ethyl acetate was added to the mixture. The organic layer was separated, and washed with water and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/3) to give the title compound (705 mg, containing ethyl acetate (5.7 w/w %), by NMR).

### E-52-7

### tert-Butyl (S)-6-benzyloxy-3-(3-formyl-4-iodo-isoxazol-5-yl)hexanoate

tert-Butyl (S)-6-benzyloxy-3-(3-hydroxymethyl-4-iodo-isoxazol-5-yl)hexanoate (705 mg, equivalent to 1.24 mmol) and chloroform (5.5 mL) were mixed. Dess-Martin periodinane (650 mg) was added to the mixture at ice temperature. The reaction mixture was stirred for 1 hr at room temperature. Aqueous 20 w/v % sodium sulfite was added to the reaction mixture at ice temperature. To the reaction mixture were added ethyl acetate and aqueous saturated sodium hydrogen carbonate. The mixture was stirred for 1 hr at room temperature. The organic layer was separated, and washed with aqueous saturated sodium hydrogen carbonate (twice) and brine in this order. The organic layer was dried over sodium sulfate. The sodium sulfate was filtered off, and the filtrate was concentrated under reduced pressure to give the title compound (645 mg).

### E-52-8

### tert-Butyl (S)-6-benzyloxy-3-(3-hydroxyiminomethyl-4-iodo-isoxazol-5-yl)hexanoate

Hydroxylammonium chloride (129 mg), THF (1.4 mL), and water (1.4 mL) were mixed. Aqueous 4 M sodium hydroxide (0.465 mL) was added dropwise to the mixture at ice temperature. A mixture of tert-butyl (S)-6-benzyloxy-3-(3-formyl-4-iodo-isoxazol-5-yl)hexanoate (645 mg) and ethanol (4.0 mL) was added dropwise to the reaction mixture at ice temperature. The reaction mixture was stirred overnight at room temperature. The pH of the reaction mixture was adjusted with aqueous 2 M hydrochloric acid to 3. To the reaction mixture were added ethyl acetate and water. The organic layer was separated, and washed with water and brine in this order, and then concentrated under reduced pressure to give the title compound (654 mg) as a crude product.

### E-52-9

### tert-Butyl (S)-6-benzyloxy-3-(chloro-hydroxyimino-methyl)-4-iodo-isoxazol-5-yl)hexanoate

tert-Butyl (S)-6-benzyloxy-3-(3-hydroxyiminomethyl-4-iodo-isoxazol-5-yl)hexanoate (654 mg), and DMF (3.0 mL) were mixed. To the mixture was added N-chlorosuccinimide (183 mg) at room temperature. The reaction mixture was stirred for 1 hr at room temperature. N-chlorosuccinimide (183 mg) was additionally added to the mixture. The reaction mixture was stirred for 1 hr at room temperature. To the reaction mixture were added toluene and water. The organic layer was separated, and washed with water and brine in this order, and the solution was directly used in the next step.

### E-52-10

### tert-Butyl (S)-6-benzyloxy-3-[5′-(1-hydroxy-2,2-dimethylpropyl)-4-iodo-[3,3′]biisoxazolyl-5-yl]-hexanoate

4,4-Dimethyl-1-pentyn-3-ol which was obtained in E-49-5 (490 mg), toluene (1.5 mL), potassium carbonate (189 mg) and water (1.0 mL) were mixed, and the mixture was heated to 100° C. A solution of tert-butyl (S)-6-benzyloxy-3-(chloro-hydroxyimino-methyl)-4-iodo-isoxazol-5-yl)hexanoate which was obtained in E-52-9 (equivalent to 1.24 mmol) in toluene was added dropwise to the reaction mixture. The resulting mixture was stirred for 20 min. To the reaction mixture were added ethyl acetate and aqueous saturated ammonium chloride. The organic layer was separated, and washed with aqueous saturated ammonium chloride and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/4) to give the title compound (719 mg).

### E-52-11

### tert-Butyl (S)-6-benzyloxy-3-[5′-(2,2-dimethylpropionyl)-4-iodo-[3,3′]biisoxazolyl-5-yl]-hexanoate

tert-Butyl (S)-6-benzyloxy-3-[5′-(1-hydroxy-2,2-dimethylpropyl)-4-iodo-[3,3′]biisoxazolyl-5-yl]-hexanoate (715 mg) and chloroform (6.0 mL) were mixed. To the mixture was added Dess-Martin periodinane (583 mg) at ice temperature. The reaction mixture was stirred for 45 min at room temperature. To the reaction mixture was added aqueous 20 w/v % sodium sulfite under water-cooling. To the reaction mixture were added ethyl acetate and aqueous saturated sodium hydrogen carbonate. The resulting mixture was stirred for 30 minutes at room temperature. The organic layer was separated, and washed with aqueous saturated sodium hydrogen carbonate (twice) and brine in this order. The organic layer was dried over sodium sulfate. The sodium sulfate was filtered off, and the filtrate was concentrated under reduced pressure to give the title compound (682 mg).

### E-52-12

### tert-Butyl (S)-6-benzyloxy-3-[5′-(1,1-difluoro-2,2-dimethyl-propyl)-4-iodo-[3,3′]biisoxazolyl-5-yl]-hexanoate

tert-Butyl (S)-6-benzyloxy-3-[5′-(2,2-dimethylpropionyl)-4-iodo-[3,3′]biisoxazolyl-5-yl]-hexanoate (682 mg), bis(2-methoxyethyl)aminosulfur trifluoride (0.84 mL) were mixed. The mixture was stirred for 3 hrs at 80° C. To the reaction mixture was added chloroform (1.0 mL) at ice temperature. The reaction mixture was poured over ice, and then aqueous saturated sodium hydrogen carbonate was added dropwise thereto at ice temperature. Ethyl acetate was added to the reaction mixture. The organic layer was separated, and washed with aqueous saturated sodium hydrogen carbonate and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/12) to give the title compound (573 mg).

### E-52-13

### tert-Butyl (S)-6-benzyloxy-3-[4-cyclopropyl-5′-(1,1-difluoro-2,2-dimethyl-propyl)[3,3′]biisoxazolyl-5-yl]-hexanoate

tert-Butyl (S)-6-benzyloxy-3-[5′-(1,1-difluoro-2,2-dimethyl-propyl)-4-iodo-[3,3′]biisoxazolyl-5-yl]-hexanoate (570 mg), 2-cyclopropyl-4,4,5,5-tetramethyl-[1,3,2]dioxaborolane (297 mg), tripotassium phosphate (751 mg), PdCl₂(PPh₃)₂ (93 mg), and DMF (5.5 mL) were mixed. The reaction mixture was degassed by bubbling argon, and the resulting mixture was stirred at 90° C. overnight. To the reaction mixture were added ethyl acetate and aqueous 1 M hydrochloric acid. The organic layer was separated, and washed with water (twice) and brine in this order. Then, the organic layer was dried over sodium sulfate. The sodium sulfate was filtered off, and the filtrate was concentrated under reduced pressure. The resulting residue was purified by silica-gel column chromatography (Eluent: ethyl acetate/hexane=1/12) to give the title compound (284 mg).

### E-52-14

### tert-Butyl (S)-6-benzyloxy-3-[4-cyclopropyl-5′-(1,1-difluoro-2,2-dimethyl-propyl)[3,3′]biisoxazolyl-5-yl]-hexanoate

tert-Butyl (S)-6-benzyloxy-3-[4-cyclopropyl-5′-(1,1-difluoro-2,2-dimethyl-propyl)[3,3′]biisoxazolyl-5-yl]-hexanoate (284 mg) and toluene (1.4 mL) were mixed. To the mixture was added trifluoroacetic acid (1.4 mL) at ice temperature. The reaction mixture was stirred for 2 hrs at room temperature, and then concentrated under reduced pressure. The resulting residue was azeotroped with toluene to give the title compound as a crude product.

### E-52-15

### (S)-6-Benzyloxy-3-[4-cyclopropyl-5′-(1,1-difluoro-2,2-dimethyl-propyl)[3,3′]biisoxazolyl-5-yl]-hexanoic acid (2-chloro-4-methyl-phenyl)-amide

tert-Butyl (S)-6-benzyloxy-3-[4-cyclopropyl-5′-(1,1-difluoro-2,2-dimethyl-propyl)[3,3′]biisoxazolyl-5-yl]-hexanoate (equivalent to 0.508 mmol), 2-chloro-4-methylphenylamine (0.075 mL), and DMF (2.5 mL) were mixed. To the mixture were added diisopropylethylamine (0.131 mL), HATU (232 mg) at ice temperature. The resulting mixture was stirred at room temperature overnight. To the reaction mixture was added aqueous 1 M hydrochloric acid at ice temperature. The resulting mixture was extracted with ethyl acetate. The organic layer was washed with water and brine in this order. The organic layer was dried over sodium sulfate. The sodium sulfate was filtered off, and the filtrate was concentrated under reduced pressure. The resulting residue was purified by preparative chromatography (Eluent: ethyl acetate/hexane=1/5) to give the title compound (279 mg).

### E-52-16

### (S)-3-[4-Cyclopropyl-5′-(1,1-difluoro-2,2-dimethyl-propyl)[3,3′]biisoxazolyl-5-yl]-6-hydroxy-hexanoic acid (2-chloro-4-methyl-phenyl)-amide

(S)-6-Benzyloxy-3-[4-cyclopropyl-5′-(1,1-difluoro-2,2-dimethyl-propyl)[3,3′]biisoxazolyl-5-yl]-hexanoic acid (2-chloro-4-methyl-phenyl)-amide (279 mg), dichloromethane (4.0 mL) were mixed. To the mixture was added boron tribromide (1 M in dichloromethane) (1.1 mL) at ice temperature. The cooling bath was removed, and the reaction mixture was stirred for 1 hr. To the reaction mixture were added aqueous saturated sodium hydrogen carbonate and ethyl acetate at ice temperature. The organic layer was separated, and washed with brine. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/1) to give the title compound (235 mg).

### E-52-17

### (S)-5-(2-Chloro-4-methyl-phenylcarbamoyl)-4-[4-cyclopropyl-5′-(1,1-difluoro-2,2-dimethylpropyl)-[3,3′]biisoxazolyl-5′-yl]pentanoic acid

(S)-3-[4′-Cyclopropyl-5-(1,1-difluoro-2,2-dimethyl-propyl)-[3,5′]biisoxazolyl-3′-yl]-6-hydroxy-hexanoic acid (4-chloro-2-fluorophenyl)amide (235 mg), acetonitrile (1.3 mL), and 1.0 M phosphate buffer (pH=6.8) (1.3 mL) were mixed. To the mixture were added 2,2,6,6-tetramethyl-1-piperidinyloxy radical (TEMPO) (5.0 mg) and sodium chlorite (73 mg) at room temperature. Aqueous sodium hypochlorite (Wako Pure Chemical Industries, 0.088 mL) was added dropwise to the reaction mixture at room temperature. The reaction mixture was stirred for 1 hr at room temperature. Aqueous 20 w/v % sodium sulfite was added to the reaction mixture at ice temperature. Ethyl acetate was added to the reaction mixture. The organic layer was separated, and washed with brine, and then dried over sodium sulfate. The sodium sulfate was filtered off, and the filtrate was concentrated under reduced pressure. The resulting residue was purified by silica-gel column chromatography (Eluent: ethyl acetate/hexane=1/2), and then azeotroped twice with ethanol to give the title compound (218 mg, containing ethanol (10.3 w/w %), by NMR).

¹H-NMR (400 MHz, DMSO-d6) 0.34-0.46 (m, 1H), 0.64-0.75 (m, 1H), 0.78-0.89 (m, 2H), 1.10 (s, 9H), 1.65-1.75 (m, 1H), 1.84-2.10 (m, 2H), 2.13-2.29 (m, 2H), 2.26 (s, 3H), 2.80-2.92 (m, 2H), 3.63-3.76 (m, 1H), 7.09 (d, J=8.00 Hz, 1H), 7.27 (s, 1H), 7.34 (s, 1H), 7.39 (d, J=8.00 Hz, 1H), 9.57 (s, 1H)

### E-52-18

### Sodium (S)-5-(2-Chloro-4-methyl-phenylcarbamoyl)-4-[4-cyclopropyl-5′-(1,1-difluoro-2,2-dimethylpropyl)-[3,3′]biisoxazolyl-5′-yl]pentanoate

((S)-5-(2-Chloro-4-methyl-phenylcarbamoyl)-4-[4-cyclopropyl-5′-(1,1-difluoro-2,2-dimethylpropyl)-[3,3′]biisoxazolyl-5′-yl]pentanoic acid (218 mg, equivalent to 0.355 mmol) and ethanol (1.5 mL) were mixed. To the mixture was added aqueous 1 M sodium hydroxide (0.355 mL) at room temperature. The resulting solution was concentrated under reduced pressure to give the title compound (175 mg).

### Example E-41

### Sodium (S)-5-(2-chloro-4-methyl-phenylcarbamoyl)-4-[4-cyclopropyl-5-(2-isobutyl-thiazol-5-yl)-isoxazol-3-yl]-pentanoate

### E-41-1

### 2-Isobutyl-thiazole-5-carboxylic acid methoxy-methyl-amide

2-Isobutyl-thiazole-5-carboxylic acid (745 mg), N,O-dimethylhydroxylamine hydrochloride salt (471 mg), HOBt.H₂O (185 mg), and acetonitrile (5.0 mL) were mixed. To the reaction mixture were added diisopropylethylamine (0.981 mL) and WSC.HCl (926 mg) at ice temperature. The resulting mixture was stirred for 7 hrs at room temperature. Water was added to the reaction mixture. The resulting mixture was extracted with ethyl acetate. The organic layer was washed with aqueous saturated sodium hydrogen carbonate, water, and aqueous saturated sodium chloride in this order. The organic layer was dried over sodium sulfate. The sodium sulfate was filtered off, and the filtrate was concentrated under reduced pressure to give the title compound (947 mg) as a crude product.

### E-41-2

### 2-Isobutyl-thiazole-5-carbaldehyde

2-Isobutyl-thiazole-5-carboxylic acid methoxy-methyl-amide (947 mg, equivalent to 4.02 mmol) and toluene (4.0 mL) were mixed. Diisobutylaluminium hydride (1.0 M in toluene) (5.21 mL) was added dropwise to the mixture at −78° C. The reaction mixture was stirred for 1 hr at −78° C. To the reaction mixture was added aqueous 2.0 M hydrochloric acid at ice temperature. The reaction mixture was stirred for 1 hr at ice temperature. To the reaction mixture was added diethyl ether. The organic layer was separated, and washed with aqueous saturated sodium hydrogen carbonate and brine in this order. The organic layer was dried over sodium sulfate. The sodium sulfate was filtered off. The filtrate was concentrated under reduced pressure to remove diethyl ether. The title compound was obtained as a toluene solution without purification.

### E-41-3

### 5-(2,2-Dibromo-vinyl)-2-isobutyl-thiazole

A solution of triphenylphosphine (2.95 g) in dichloromethane (9.4 mL) was added dropwise to a solution of carbon tetrabromide (1.87 g) in dichloromethane (4.7 mL) at ice temperature. The reaction mixture was stirred for min at ice temperature. A solution of 2-isobutyl-thiazole-5-carbaldehyde which was obtained in E-41-2 in toluene (equivalent to 4.02 mmol) and dichloromethane (9.4 mL) were mixed. The resulting mixture was added a opwise to the reaction mixture at ice temperature. The reaction mixture was stirred for 45 min at ice temperature. To the reaction mixture were added aqueous saturated sodium hydrogen carbonate and diethyl ether. The organic layer was separated, and washed with brine, and silica gel (2.0 g) was added thereto. The silica gel was filtered off with washing with diethyl ether, and the filtrate was concentrated under reduced pressure. The resulting residue was purified by silica-gel column chromatography (Eluent: diethyl ether/hexane=1/15) to give the title compound (628 mg).

### E-41-4

### tert-Butyl (S)-3-(3-benzyloxy-propyl)-6-(2-isobutyl-thiazol-5-yl)-4-oxo-5-hexynoate

5-(2,2-dibromo-vinyl)-2-isobutyl-thiazole (625 mg) THF (3.0 mL) were mixed. n-Butyllithium (2.69 M in hexane) (1.43 mL) was added dropwise to the mixture at −78° C. The reaction mixture was stirred for 20 min at −78° C. tert-Butyl (S)-6-benzyloxy-3-(methoxy-methylcarbamoyl)-hexanoate which was obtained in E-58-7 (597 mg, equivalent to 1.63 mmol) was added to the reaction mixture at −78° C. The reaction mixture was stirred for 30 minutes at ice temperature. To the reaction mixture were added aqueous 2.0 M hydrochloric acid and ethyl acetate at ice temperature. The organic layer was separated, and washed with water and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/7) to give the title compound (433 mg).

### E-41-5

### tert-Butyl (S)-3-(3-benzyloxy-propyl)-6-(2-isobutyl-thiazol-5-yl)-4-methoxyimino-5-hexynoate

tert-Butyl (S)-3-(3-benzyloxy-propyl)-6-(2-isobutyl-thiazol-5-yl)-4-oxo-5-hexynoate (425 mg) and ethanol (4.0 mL) were mixed. To the mixture were added sodium carbonate (480 mg), O-methylhydroxylammonium chloride (378 mg) at room temperature. The reaction mixture was stirred for 1.5 hrs at 80° C. To the reaction mixture were added ethyl acetate and water at room temperature. The organic layer was separated, and washed with water and brine in this order. The organic layer was dried over sodium sulfate. The sodium sulfate was filtered off, and the filtrate was concentrated under reduced pressure to give the title compound (433 mg) as a crude product.

### E-41-6

### tert-Butyl (S)-6-benzyloxy-3-[4-iodo-5-(2-isobutyl-thiazol-5-yl)-isoxazol-3-yl]-hexanoate

tert-Butyl (S)-3-(3-benzyloxy-propyl)-6-(2-isobutyl-thiazol-5-yl)-4-methoxyimino-5-hexynoate (438 mg) and dichloromethane (6.5 mL) were mixed. To the mixture was added iodine monochloride (1.0 M in dichloromethane) (0.88 mL) at ice temperature. The reaction mixture was stirred for 40 min at ice temperature, and then aqueous 20 w/v % sodium sulfite was added thereto. To the reaction mixture was added dichloromethane. The organic layer was separated, and dried over sodium sulfate. The sodium sulfate was filtered off, and the filtrate was concentrated under reduced pressure. The resulting residue was purified by silica-gel column chromatography (Eluent: ethyl acetate/hexane=1/5) to give the title compound (460 mg).

### E-41-7

### tert-Butyl (S)-6-benzyloxy-3-[4-cyclopropyl-5-(2-isobutyl-thiazol-5-yl)-isoxazol-3-yl]-hexanoate

tert-Butyl (S)-6-benzyloxy-3-[4-iodo-5-(2-isobutyl-thiazol-5-yl)-isoxazol-3-yl]-hexanoate (460 mg), 2-cyclopropyl-4,4,5,5-tetramethyl-[1,3,2]dioxaborolane (253 mg), and DMF (4.0 mL) were mixed. To the mixture were added tripotassium phosphate (639 mg) and PdCl₂(PPh₃)₂ (79 mg). The reaction mixture was degassed by bubbling argon, and was stirred for 7.5 hrs at 90° C. To the reaction mixture were added ethyl acetate, aqueous 1 M hydrochloric acid. The organic layer was separated, and washed with water and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/7) to give the title compound (365 mg).

### E-41-8

### (S)-6-Benzyloxy-3-[4-cyclopropyl-5-(2-isobutyl-thiazol-5-yl)-isoxazol-3-yl]-hexanoic acid

tert-Butyl (S)-6-benzyloxy-3-[4-cyclopropyl-5-(2-isobutyl-thiazol-5-yl)-isoxazol-3-yl]-hexanoate (365 mg) and toluene (2.1 mL) were mixed. To the mixture was added trifluoroacetic acid (0.70 mL) at ice temperature. The reaction mixture was stirred at room temperature overnight, and concentrated under reduced pressure. The resulting residue was azeotroped with toluene to give the title compound as a crude product.

### E-41-9

### (S)-6-Benzyloxy-3-[4-cyclopropyl-5-(2-isobutyl-thiazol-5-yl)-isoxazol-3-yl]-hexanoic acid (2-chloro-4-methyl-phenyl)-amide

(S)-6-benzyloxy-3-[4-cyclopropyl-5-(2-isobutyl-thiazol-5-yl)-isoxazol-3-yl]-hexanoic acid (equivalent to 0.70 mmol), 2-chloro-4-methyl-phenylamine (0.103 mL), and DMF (3.0 mL) were mixed. To the mixture were added diisopropylethylamine (0.183 mL) and HATU (320 mg) at ice temperature. The resulting mixture was stirred overnight at room temperature. Diethyl ether and water were added to the reaction mixture. The organic layer was separated, and washed with water and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/4) to give the title compound (385 mg).

### E-41-10

### (S)-3-[4-Cyclopropyl-5-(2-isobutyl-thiazol-5-yl)-isoxazol-3-yl]-6-hydroxy-hexanoic acid (2-chloro-4-methyl-phenyl)amide

(S)-6-Benzyloxy-3-[4-cyclopropyl-5-(2-isobutyl-thiazol-5-yl)-isoxazol-3-yl]-hexanoic acid (2-chloro-4-methyl-phenyl)-amide (385 mg) and dichloromethane (5.0 mL) were mixed. To the mixture was added boron tribromide (1 M in dichloromethane) (1.1 mL) at ice temperature. The cooling bath was removed, and the reaction mixture was stirred for 40 minutes. To the reaction mixture were added aqueous saturated sodium hydrogen carbonate and ethyl acetate at ice temperature. The organic layer was separated, and washed with brine. The organic layer was dried over sodium sulfate. The sodium sulfate was filtered off, and the filtrate was concentrated under reduced pressure. The resulting residue was purified by silica-gel column chromatography (Eluent: ethyl acetate/hexane=2/1) to give a crude product (123 mg). The crude product was purified by preparative chromatography (Eluent: ethyl acetate/hexane=2/1) to give the title compound (115 mg, containing ethyl acetate (17.8 w/w %), by NMR).

### E-41-11

### (S)-5-(2-Chloro-4-methyl-phenylcarbamoyl)-4-[4-cyclopropyl-5-(2-isobutyl-thiazol-5-yl)-isoxazol-3-yl]-pentanoic acid

(S)-3-[4-cyclopropyl-5-(2-isobutyl-thiazol-5-yl)-isoxazol-3-yl]-6-hydroxy-hexanoic acid (2-chloro-4-methyl-phenyl)amide (95 mg), acetonitrile (0.7 mL), and 1.0 M phosphate buffer (pH=6.8) (0.7 mL) were mixed. To the mixture were added 2,2,6,6-tetramethyl-1-piperidinyloxy radical (TEMPO) (3.0 mg), sodium chlorite (43 mg), and aqueous sodium hypochlorite (Wako Pure Chemical Industries, 0.038 mL) at ice temperature. The reaction mixture was stirred for 1 hr at room temperature. Aqueous 20 w/v % sodium sulfite and ethyl acetate was added to the reaction mixture at ice temperature. The organic layer was separated, and washed with water and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/2), and then azeotroped with ethanol to give the title compound (98.5 mg, containing ethanol (17.9 w/w %), by NMR).

¹H-NMR (400 MHz, CDCl3) 0.41-0.51 (m, 1H), 0.63-0.73 (m, 1H), 0.94-1.08 (m, 2H), 1.00 (d, J=6.80 Hz, 6H), 1.50-1.61 (m, 1H), 2.06-2.30 (m, 3H), 2.25 (s, 3H), 2.36-2.51 (m, 2H), 2.78 (dd, J=14.80, 5.60 Hz, 1H), 2.91 (d, J=7.20 Hz, 2H), 3.04 (dd, J=14.80, 9.60 Hz, 1H), 3.67-3.83 (m, 1H), 7.00 (d, J=8.40 Hz, 1H), 7.12 (s, 1H), 7.70 (s, 1H), 8.05 (d, J=8.40 Hz, 1H), 8.11 (s, 1H)

### E-41-12

### Sodium (S)-5-(2-chloro-4-methyl-phenylcarbamoyl)-4-[4-cyclopropyl-5-(2-isobutyl-thiazol-5-yl)-isoxazol-3-yl]-pentanoate

(S)-5-(2-Chloro-4-methyl-phenylcarbamoyl)-4-[4-cyclopropyl-5-(2-isobutyl-thiazol-5-yl)-isoxazol-3-yl]-pentanoic acid (98.5 mg, equivalent to 0.157 mmol), ethanol (1.0 mL) were mixed. To the mixture was added aqueous 1 M sodium hydroxide (0.157 mL) at room temperature. The resulting solution was concentrated under reduced pressure to give the title compound (74.7 mg).

### Example E-53

### Sodium (R)-4-(2-chloro-4-methyl-phenylcarbamoyl)-3-[4′-cyclopropyl-5-(1,1-difluoro-2,2-dimethylpropyl)-[3,5′]biisoxazolyl-3′-yl]-butyrate

### E-53-1

### (S)-4-benzyl-3-(4-benzyloxy-butyryl)-oxazolidin-2-one

4-Benzyloxy-butyric acid (238 g) and chloroform (1300 mL) were mixed. To the mixture was added (S)-4-benzyl-2-oxazolidinone (217 g). To the mixture were added 4-dimethylaminopyridine (45.0 g) and WSC.HCl (282 g) at ice temperature. The resulting mixture was stirred overnight at room temperature. The reaction mixture was concentrated under reduced pressure. Then, to the residue were added toluene (2000 mL) and aqueous 1 M hydrochloric acid (1500 mL). The organic layer was separated, and washed with aqueous saturated sodium hydrogen carbonate (2000 mL), aqueous saturated sodium chloride (1500 mL) in this order. The organic layer was dried over sodium sulfate. The sodium sulfate was filtered off and the filtrate was concentrated under reduced pressure to give the title compound (425 g, containing toluene (3.2 w/w %), by NMR) as a crude product.

### E-53-2

### tert-Butyl (R)-3-((S)-4-benzyl-2-oxo-oxazolidine-3-carbonyl)-5-benzyloxy-pentanoate

(S)-4-benzyl-3-(4-benzyloxy-butyryl)-oxazolidin-2-one (425 g, equivalent to 1.16 mol) and THF (1600 mL) were mixed. Sodium hexamethyldisilazide (1.9 M in THF) (702 mL) was added dropwise to the mixture at −78° C. tert-Butyl bromoacetate (275 mL) was added dropwise to the reaction mixture at −78° C. The resulting mixture was stirred for 2 hrs. The temperature of the reaction mixture was raised to −15° C. over 1.5 hrs, and then N,N,N′-trimethylethylenediamine (120 mL) was added dropwise to the mixture. To the reaction mixture were added water (1640 mL) and toluene (2460 mL) at ice temperature. The organic layer was separated, and washed with aqueous 20 w/v % citric acid (2460 mL), water (1640 mL), aqueous saturated sodium hydrogen carbonate (2050 mL), and aqueous saturated sodium chloride (1640 mL) in this order. The organic layer was concentrated under reduced pressure. The resulting residue (648.4 g, containing toluene (13 w/w %), by NMR) and methanol (2260 mL) were mixed. To the mixture was added activated carbon (85 g). The resoling mixture was stirred for 2 hrs at 75° C. The bath was removed, and then the mixture was stirred for 1 hr. The activated carbon was filtered off with washing with methanol (1130 mL). The filtrate was concentrated under reduced pressure to give the title compound (570 g, containing methanol (4.3 w/w %), by NMR) as a crude product.

### E-53-3

### tert-Butyl (R)-3-((S)-4-benzyl-2-oxo-oxazolidine-3-carbonyl)-5-hydroxy-pentanoate

tert-Butyl (R)-3-((S)-4-benzyl-2-oxo-oxazolidine-3-carbonyl)-5-benzyloxy-pentanoate (500 g), ethyl acetate (750 mL), and THF (1510 mL) were mixed. To the mixture was added 20 w/w % palladium hydroxide (50 g). The reaction mixture was stirred for 4.5 hrs under hydrogen atmosphere (1 atm). Then, the reaction container was charged with nitrogen gas. The palladium hydroxide was filtered off with washing with ethyl acetate (1000 mL). The filtrate was concentrated under reduced pressure to give the title compound (456 g, containing ethyl acetate (7.0 w/w %), by NMR) as a crude product.

### E-53-4

### tert-Butyl (R)-3-((S)-4-benzyl-2-oxo-oxazolidine-3-carbonyl)-5-(tert-butyl-diphenyl-silanyloxy)-pentanoate

tert-Butyl (R)-3-((S)-4-benzyl-2-oxo-oxazolidine-3-carbonyl)-5-hydroxy-pentanoate (431 g, equivalent to 1.06 mol) and DMF (2000 mL) were mixed. To the mixture were added imidazole (160 g) and tert-butylchlorodiphenylsilane (287 mL) at ice temperature. The reaction mixture was stirred for 1 hr at room temperature. To the mixture were added water (1200 mL) and toluene (2300 mL). The organic layer was separated, and washed with aqueous 20 w/v % citric acid (1600 mL), water (2000 mL), and aqueous 10 w/v % sodium chloride (1600 mL) in this order. The organic layer was dried over sodium sulfate. The sodium sulfate was filtered off and the filtrate was concentrated under reduced pressure to give the title compound (744.2 g, containing toluene (17 w/w %), by NMR) as a crude product.

### E-53-5

### tert-Butyl (R)-2-[2-(tert-butyl-diphenyl-silanyloxy)-ethyl]-succinate

Lithium hydroxide monohydrate (58 g), THF (1300 mL) and water (600 mL) were mixed. Aqueous 30 w/w % hydrogen peroxide (256 mL) was added dropwise to the mixture at ice temperature. The reaction mixture was stirred for 1 hr at ice temperature. A solution of tert-butyl (R)-3-((S)-4-benzyl-2-oxo-oxazolidine-3-carbonyl)-5-(tert-butyl-diphenyl-silanyloxy)-pentanoate (744 g, equivalent to 1.06 mol) in THF (1200 mL) was added dropwise to the reaction mixture at ice temperature. The reaction mixture was stirred for 2 hrs at room temperature, then aqueous 20 w/v % sodium hydrogen sulfite (1632 mL) was added dropwise thereto at ice temperature. To the reaction mixture was added ethyl acetate (3600 mL). The organic layer was separated, and washed with water (2000 mL) and aqueous 10 w/v % sodium chloride (2000 mL) in this order, and concentrated under reduced pressure. The resulting residue (706.7 g), hexane (3500 mL), aqueous 1 M sodium carbonate (2800 mL) were mixed. The aqueous layer was separated, and washed with hexane (1500 mL). Aqueous 6 M hydrochloric acid (865 mL) was added dropwise to the aqueous layer at ice temperature. The aqueous layer was extracted with ethyl acetate (2200 mL). The organic layer was washed with water (2200 mL) and aqueous 10 w/v % sodium chloride (1500 mL) in this order. The organic layer was dried over sodium sulfate. The sodium sulfate was filtered off, and the filtrate was concentrated under reduced pressure. Diisopropylether (1100 mL) and hexane (1600 mL) were added thereto. The resulting mixture was stirred at room temperature. The resultant precipitate was collected by filtration to give the title compound (437.4 g, containing diisopropylether etc. by NMR).

### E-53-6

### tert-Butyl (R)-5-(tert-butyl-diphenyl-silanyloxy)-3-(methoxy-methyl-carbamoyl)-pentanoate

tert-Butyl (R)-2-[2-(tert-butyl-diphenyl-silanyloxy)-ethyl]-succinate (437 g, equivalent to 874 mmol), triethylamine (171 mL), and DMF (2000 mL) were mixed. To the mixture were added N,O-dimethylhydroxylamine hydrochloride salt (111 g), HOBt.H₂O (161 g), and WSC.HCl (201 g) at ice temperature. The reaction mixture was stirred overnight at room temperature. To the reaction mixture were added water (800 mL) and hexane (2400 mL). The organic layer was separated, and washed with water (1200 mL) and aqueous 10 w/v % sodium chloride (1200 mL) in this order: The combined aqueous layer was re-extracted with hexane (2400 mL). The combined organic layer was dried over sodium sulfate. The sodium sulfate was filtered off, and the filtrate was concentrated under reduced pressure to give the title compound (425 g, containing hexane (1.8 w/w %), by NMR) as a crude product.

### E-53-7

### tert-Butyl 5-(tert-butyl-diphenyl-silanyloxy)-3-(R)-formyl-pentanoate

tert-Butyl (R)-5-(tert-butyl-diphenyl-silanyloxy)-3-(methoxy-methyl-carbamoyl)-pentanoate (30.9 g, equivalent to 60.0 mmol) and THF (150 mL) were mixed. Diisobutylaluminium hydride (1.0 M in toluene) (78.0 mL) was added dropwise to the mixture at −78° C. The reaction mixture was stirred for 30 minutes at −78° C. The reaction mixture was added dropwise to aqueous 1.0 M sulfuric acid (114 mL) at ice temperature. To the reaction mixture was added toluene. The organic layer was separated, and washed with aqueous 10 w/v % potassium hydrogen sulfate and water in this order, then concentrated under reduced pressure to give the title compound (31.5 g) as a crude product.

### E-53-8

### tert-Butyl 5-(tert-butyl-diphenyl-silanyloxy)-3-((R)-hydroxyimino-methyl)-pentanoate

Hydroxylammonium chloride (7.06 g), ethanol (122 mL), and water (35 mL) were mixed. Aqueous 4 M sodium hydroxide (23.4 mL) was added dropwise to the mixture at ice temperature. A mixture of tert-Butyl 5-(tert-butyl-diphenyl-silanyloxy)-3-(R)-formyl-pentanoate (40.9 g, equivalent to 78.1 mmol), ethanol (18 mL), and THF (35 mL) was added dropwise to the reaction mixture at ice temperature. The reaction mixture was stirred for 3 hrs at ice temperature. To the reaction mixture was added toluene (210 mL). The organic layer was separated, and washed with water and brine in this order. The organic layer was dried over magnesium sulfate. The magnesium sulfate was filtered off, and the filtrate was concentrated under reduced pressure to give the title compound (40.7 g, containing toluene (13 w/w %), by NMR) as a crude product.

### E-53-9

### tert-Butyl 5-(tert-butyl-diphenyl-silanyloxy)-3-((R)-chloro-hydroxyimino-methyl)-pentanoate

tert-Butyl 5-(tert-butyl-diphenyl-silanyloxy)-3-((R)-hydroxyimino-methyl)-pentanoate (23.0 g, equivalent to 43.9 mmol) and DMF (100 mL) were mixed. To the mixture was added N-chlorosuccinimide (7.03 g) at ice temperature. The reaction mixture was stirred for 2 hrs at room temperature. To the reaction mixture were added water and aqueous saturated sodium chloride at ice temperature. The reaction mixture was extracted with toluene (120 mL, 60 mL). The organic layer was washed with water and brine in this order. The organic layer was concentrated under reduced pressure until being reduced to approximately 120 mL in volume, and the resulting solution was used in the next step.

### E-53-10

### tert-Butyl (R)-5-(tert-butyl-diphenyl-silanyloxy)-3-(5-hydroxymethyl-isoxazol-3-yl)-pentanoate

Propargyl alcohol (3.20 g), toluene (60 mL), potassium carbonate (6.37 g), and water (30 mL) were mixed. The mixture was heated to 110° C. A solution of tert-butyl (5-(tert-butyl-diphenyl-silanyloxy)-3-((R)-chloro-hydroxyimino-methyl)-pentanoate which was obtained in E-53-9 in toluene (equivalent to 43.9 mmol) was added dropwise to the reaction mixture. The resulting mixture was stirred for 30 minutes. The resulting organic layer was separated, and washed with water and aqueous saturated sodium chloride. The organic layer was concentrated under reduced pressure to give the title compound (26.0 g) as a crude product.

### E-53-11

### tert-Butyl (R)-3-(5-acetoxymethyl-isoxazol-3-yl)-5-(tert-butyl-diphenyl-silanyloxy)-pentanoate

tert-Butyl (R)-5-(tert-butyl-diphenyl-silanyloxy)-3-(5-hydroxymethyl-isoxazol-3-yl)-pentanoate (26.0 g, equivalent to 43.9 mmol), triethylamine (11.0 mL), and chloroform (130 mL) were mixed. Acetyl chloride (4.39 mL) was added dropwise to the mixture at ice temperature. The reaction mixture was stirred for 1 hr at ice temperature, and water was added thereto. The reaction mixture was extracted with chloroform. The organic layer was washed with water and aqueous saturated sodium chloride in this order. The organic layer was dried over magnesium sulfate. The magnesium sulfate was filtered off, and the filtrate was concentrated under reduced pressure to give the title compound (30.1 g) as a crude product.

### E-53-12

### tert-Butyl (R)-3-(5-acetoxymethyl-isoxazol-3-yl)-5-hydroxy-pentanoate

tert-Butyl (R)-5-(tert-butyl-diphenyl-silanyloxy)-3-(5-hydroxymethyl-isoxazol-3-yl)-pentanoate (17.62 g) and THF (150 mL) were mixed. To the mixture were added acetic acid/water=4/1 (4.8 mL) and tetrabutylammonium fluoride (1 M in THF) (48.3 mL) at ice temperature. The resulting mixture was stirred overnight at room temperature. To the reaction mixture was added water at ice temperature, and the resulting mixture was extracted with ethyl acetate. The organic layer was washed with aqueous saturated sodium chloride, and dried with magnesium sulfate. The magnesium sulfate was filtered off, and the filtrate was concentrated under reduced pressure, and the resulting residue was purified by silica-gel column chromatography (Eluent: ethyl acetate/hexane=1/1) to give the title compound (11.0 g, containing ethyl acetate (5.0 w/w %), by NMR).

### E-53-13

### Mono-tert-butyl (R)-3-(5-acetoxymethyl-isoxazol-3-yl)-pentanedicarboxylate

tert-Butyl (R)-3-(5-acetoxymethyl-isoxazol-3-yl)-5-hydroxy-pentanoate (2,4-dichloro-phenyl)-amide (10.4 g), acetonitrile (60 mL), and 1.0 M phosphate buffer (pH=6.8) (40 mL) were mixed. To the mixture were added 2,2,6,6-tetramethyl-1-piperidinyloxy radical (TEMPO) (260 mg) and sodium chlorite (7.50 g) at ice temperature. Aqueous sodium hypochlorite (Wako Pure Chemical Industries, 4.0 mL) was added dropwise to the reaction mixture at ice temperature. The reaction mixture was stirred for 2 hrs at room temperature, and then aqueous sodium sulfite was added thereto at ice temperature. The pH of the reaction mixture was adjusted with aqueous sodium hydrogen sulfate to 6. To the reaction mixture was added ethyl acetate. The organic layer was separated, and washed with water and brine, and dried with magnesium sulfate. The magnesium sulfate was filtered off, and the filtrate was concentrated under reduced pressure. The residue was azeotroped with toluene to give the title compound (12.2 g) as a crude product.

### E-53-14

### Mono-tert-butyl (R)-3-(5-hydroxymethyl-isoxazol-3-yl)-pentanedicarboxylate

Mono-tert-butyl (R)-3-(5-acetoxymethyl-isoxazol-3-yl)-pentanedicarboxylate (12.2 g, equivalent to 33.2 mmol), methanol (50 mL), and water (5 mL) were mixed. To the mixture was added potassium carbonate (9.18 g) at ice temperature. The reaction mixture was stirred for 1.5 hrs at room temperature, and aqueous 1 M hydrochloric acid (133 mL) was added thereto. The reaction mixture was extracted with chloroform (four times), and the combined organic layer was dried with magnesium sulfate. The magnesium sulfate was filtered off, and the filtrate was concentrated under reduced pressure. The residue was azeotroped with toluene to give the title compound (10.9 g) as a crude product.

### E-53-15

### Benzyl tert-butyl (R)-3-(5-hydroxymethyl-isoxazol-3-yl)-pentanedicarboxylate

Mono-tert-butyl (R)-3-(5-hydroxymethyl-isoxazol-3-yl)-pentanedicarboxylate (10.9 g, equivalent to 33.2 mmol) and DMF (50 mL) were mixed, and then benzyl bromide (4.73 mL) was added thereto. To the reaction mixture was added potassium hydrogen carbonate (4.65 g) at ice temperature. The resulting mixture was stirred overnight at room temperature. To the reaction mixture was added water at ice temperature. The resulting mixture was extracted with ethyl acetate. The organic layer was washed with water and aqueous saturated sodium chloride in this order. The organic layer was dried over magnesium sulfate. The magnesium sulfate was filtered off, and the filtrate was concentrated under reduced pressure, and the resulting residue was purified by silica-gel column chromatography (Eluent: ethyl acetate/hexane=1/2) to give the title compound (11.0 g).

### E-53-16

### Benzyl tert-butyl (R)-3-(5-hydroxymethyl-4-iodo-isoxazol-3-yl)-pentanedicarboxylate

Benzyl tert-butyl (R)-3-(5-hydroxymethyl-isoxazol-3-yl)-pentanedicarboxylate (11.0 g) and acetonitrile (50 mL) were mixed. To the mixture were added N-iodosuccinimide (11.2 g) and ammonium cerium(IV) nitrate (3.21 g) under water-cooling. The reaction mixture was stirred at room temperature overnight. Aqueous 10 w/v % sodium sulfite (121 mL) was added dropwise to the mixture at ice temperature. The reaction mixture was filtered using Celite with washing with ethyl acetate. To the filtrate was added ethyl acetate. The organic layer was separated, and washed with water and brine, and dried with magnesium sulfate. The magnesium sulfate was filtered off, and the filtrate was concentrated under reduced pressure. The resulting residue was purified by silica-gel column chromatography (Eluent: ethyl acetate/hexane=1/3) to give the title compound (3.90 g).

### E-53-17

### Benzyl tert-butyl (R)-3-(4-cyclopropyl-5-hydroxymethyl-isoxazol-3-yl)-pentanedicarboxylate

Benzyl tert-butyl (R)-3-(5-hydroxymethyl-4-iodo-isoxazol-3-yl)-pentanedicarboxylate (3.90 g), 2-cyclopropyl-4,4,5,5-tetramethyl-[1,3,2]dioxaborolane (2.06 mL), tripotassium phosphate (4.95 g), and DMF (18 mL) were mixed. To the reaction mixture was added PdCl₂(PPh₃)₂ (546 mg) under argon atmosphere. The reaction mixture was stirred at 90° C. overnight under argon atmosphere. To the reaction mixture was added ethyl acetate, and the resulting mixture was filtered using Celite. The filtrate was washed with water (twice) and brine in this order, and then dried with magnesium sulfate. The magnesium sulfate was filtered off, and the filtrate was concentrated under reduced pressure. The resulting residue was purified by silica-gel column chromatography (Eluent: ethyl acetate/hexane=1/3) to give the title compound (595 mg).

### E-53-18

### Benzyl tert-butyl (R)-3-(4-cyclopropyl-5-formyl-isoxazol-3-yl)-pentanedicarboxylate

Benzyl tert-butyl (R)-3-(4-cyclopropyl-5-hydroxymethyl-isoxazol-3-yl)-pentanedicarboxylate (596 mg), chloroform (5.0 mL) were mixed. To the mixture was added Dess-Martin periodinane (728 mg) at ice temperature. The reaction mixture was stirred for 30 minutes at room temperature, and added aqueous 20 w/v % sodium sulfite (10 mL) at ice temperature. To the reaction mixture were added ethyl acetate, water, aqueous saturated sodium hydrogen carbonate. The resulting mixture was stirred for 1 hr at room temperature. The organic layer was separated, washed with brine, and dried with magnesium sulfate. The magnesium sulfate was filtered off, and the filtrate was concentrated under reduced pressure to give the title compound (612 mg) as a crude product.

### E-53-19

### Benzyl tert-butyl (R)-3-(4-cyclopropyl-5-(hydroxyimino-methyl)-isoxazol-3-yl)-pentanedicarboxylate

Benzyl tert-butyl (R)-3-(4-cyclopropyl-5-formyl-isoxazol-3-yl)-pentanedicarboxylate (612 mg), ethanol (4.0 mL), THF (1.0 mL), and water (1.0 mL) were mixed. Hydroxylammonium chloride (120 mg), aqueous 4 M sodium hydroxide (0.43 mL) were added to the reaction mixture at ice temperature. The reaction mixture was stirred at room temperature overnight. To the mixture were added ethyl acetate and water. The organic layer was separated, and washed with water and brine in this order. The organic layer was dried over magnesium sulfate. The magnesium sulfate was filtered off, and the filtrate was concentrated under reduced pressure to give the title compound (626 mg) as a crude product.

### E-53-20

### Benzyl tert-butyl (R)-3-(4-cyclopropyl-5-(chloro-hydroxyimino-methyl)-isoxazol-3-yl)-pentanedicarboxylate

Benzyl tert-butyl (R)-3-(4-cyclopropyl-5-(hydroxyimino-methyl)-isoxazol-3-yl)-pentanedicarboxylate (626 mg), DMF (4.0 mL) were mixed. To the mixture was added N-chlorosuccinimide (200 mg) at room temperature. The reaction mixture was stirred for 1 hr at room temperature, and N-chlorosuccinimide (200 mg) was added again. To the reaction mixture were added toluene and water. The organic layer was separated, and washed with water, and the solution was directly used in the next step.

### E-53-21

### Benzyl tert-butyl (R)-3-[4′-cyclopropyl-5-(1-hydroxy-2,2-dimethyl-propyl)-[3,5′]biisoxazolyl-3′-yl]-pentanedicarboxylate

4,4-dimethyl-1-pentyn-3-ol (0.321 mL) which was obtained in a similar way to E-49-5, toluene (1.0 mL), potassium carbonate (207 mg), water (1.0 mL) were mixed. The mixture was heated to 120° C. A solution of benzyl tert-butyl (R)-3-(4-cyclopropyl-5-(chloro-hydroxyimino-methyl)-isoxazol-3-yl)-pentanedicarboxylate in toluene which was obtained in E-53-20 (equivalent to 1.43 mmol) was added dropwise to the reaction mixture, and the resulting mixture was stirred for 30 minutes. To the reaction mixture were added water and ethyl acetate. The organic layer was separated, washed with brine, and dried with magnesium sulfate. The magnesium sulfate was filtered off, and the filtrate was concentrated under reduced pressure. The resulting residue was purified by silica-gel column chromatography (Eluent: ethyl acetate/hexane=1/1) to give the title compound (712 mg).

### E-53-22

### Benzyl tert-butyl (R)-3-[4′-cyclopropyl-5-(2,2-dimethyl-propionyl)-[3,5′]biisoxazolyl-3′-yl]-pentanedicarboxylate

Benzyl tert-butyl (R)-3-[4′-cyclopropyl-5-(1-hydroxy-2,2-dimethyl-propyl)-[3,5′]biisoxazolyl-3′-yl]-pentanedicarboxylate (712 mg), chloroform (4.0 mL) were mixed. To the mixture was added Dess-Martin periodinane (672 mg) at ice temperature. The reaction mixture was stirred for 1 hr at room temperature, and to the mixture was added aqueous 20 w/v % sodium sulfite at ice temperature. The reaction mixture was stirred for 30 minutes at room temperature, and then ethyl acetate and water were added thereto. The organic layer was separated, washed with brine, and dried with magnesium sulfate. The magnesium sulfate was filtered off, and the filtrate was concentrated under reduced pressure to give the title compound (770 mg) as a crude product.

### E-53-23

### Benzyl tert-butyl (R)-3-[4′-cyclopropyl-5-(1,1-difluoro-2,2-dimethyl-propyl)-[3,5′]biisoxazolyl-3′-yl]-pentanedicarboxylate

Benzyl tert-butyl (R)-3-[4′-cyclopropyl-5-(2,2-dimethyl-propionyl)-[3,5′]biisoxazolyl-3′-yl]-pentanedicarboxylate (769 mg) and bis(2-methoxyethyl)aminosulfur trifluoride (0.973 mL) were mixed. The mixture was stirred for 2 hrs 80° C. To the reaction mixture was added dichloromethane at ice temperature. The reaction mixture was added to aqueous sodium hydrogen carbonate at ice temperature. Ethyl acetate was added to the mixture. The organic layer was separated, and washed with water and brine in this order. The organic layer was dried over magnesium sulfate. The magnesium sulfate was filtered off, and the filtrate was concentrated under reduced pressure. The resulting residue was purified by silica-gel column chromatography (Eluent: ethyl acetate/hexane=1/4) to give the title compound (589 mg).

### E-53-24

### Mono-tert-butyl (R)-3-[4′-cyclopropyl-5-(1,1-difluoro-2,2-dimethyl-propyl)-[3,5′]biisoxazolyl-3′-yl]-pentanedicarboxylate

Benzyl tert-butyl (R)-3-[4′-cyclopropyl-5-(1,1-difluoro-2,2-dimethyl-propyl)-[3,5′]biisoxazolyl-3′-yl]-pentanedicarboxylate (589 mg), and THF (5.0 mL) were mixed. To the mixture was added 20 w/w % palladium carbon (300 mg). The reaction mixture was stirred for 5.5 hrs under hydrogen atmosphere (0.4 MPa). Then, the reaction container was charged with nitrogen gas. The palladium carbon was filtered off with washing with THF. The filtrate was concentrated under reduced pressure to give the title compound (546 mg) as a crude product.

### E-53-25

### tert-Butyl (R)-4-(2-chloro-4-methyl-phenylcarbamoyl)-3-[4′-cyclopropyl-5-(1,1-difluoro-2,2-dimethyl-propyl)-[3,5′]biisoxazolyl-3′-yl]-butyrate

Mono-tert-butyl (R)-3-[4′-cyclopropyl-5-(1,1-difluoro-2,2-dimethyl-propyl)-[3,5′]biisoxazolyl-3′-yl]-pentanedicarboxylate (200 mg) and DMF (2.0 mL) were mixed. To the mixture were added diisopropylethylamine (0.080 mL), 2-chloro-4-methyl-phenylamine (0.057 mL), and HATU (192 mg) at room temperature. The resulting mixture was stirred overnight. To the reaction mixture were added water and ethyl acetate. The organic layer was separated, and washed with aqueous saturated ammonium chloride, aqueous saturated sodium hydrogen carbonate, and brine in this order. The organic layer was dried over magnesium sulfate. The magnesium sulfate was filtered off, and the filtrate was concentrated under reduced pressure. The resulting residue was purified by silica-gel column chromatography (Eluent: ethyl acetate/hexane=1/4) to give the title compound (182 mg).

### E-53-26

### (R)-4-(2-chloro-4-methyl-phenylcarbamoyl)-3-[4′-cyclopropyl-5-(1,1-difluoro-2,2-dimethyl-propyl)-[3,5′]biisoxazolyl-3′-yl]-butyric acid

tert-Butyl (R)-4-(2-chloro-4-methyl-phenylcarbamoyl)-3-[4′-cyclopropyl-5-(1,1-difluoro-2,2-dimethyl-propyl)-[3,5′]biisoxazolyl-3′-yl]-butyrate (182 mg) and toluene (2.0 mL) were mixed. To the mixture was added trifluoroacetic acid (2.0 mL) at room temperature. The reaction mixture was stirred for 30 min at room temperature and concentrated under reduced pressure. The residue was azeotroped with toluene (twice). The resulting residue was purified by preparative chromatography (Eluent: methanol/chloroform=1/10) to give the title compound (162 mg, containing chloroform (1.8 w/w %), by NMR).

¹H-NMR (400 MHz, CDCl3) 0.71-0.78 (m, 2H), 1.05-1.09 (m, 2H), 1.14 (s, 9H), 1.72-1.79 (m, 1H), 2.28 (s, 3H), 2.87-3.10 (m, 4H), 4.07-4.14 (m, 1H), 6.87 (s, 1H), 7.04 (d, J=8.38 Hz, 1H), 7.16 (s, 1H), 7.71 (s, 1H), 8.12 (d, J=8.16 Hz, 1H)

### E-53-27

### Sodium (R)-4-(2-chloro-4-methyl-phenylcarbamoyl)-3-[4′-cyclopropyl-5-(1,1-difluoro-2,2-dimethylpropyl)-[3,5′]biisoxazolyl-3′-yl]-butyrate

(R)-4-(2-Chloro-4-methyl-phenylcarbamoyl)-3-[4′-cyclopropyl-5-(1,1-difluoro-2,2-dimethyl-propyl)-[3,5′]biisoxazolyl-3′-yl]-butyric acid (162.1 mg, equivalent to 0.297 mmol), ethanol were mixed. To the mixture was added aqueous 1 M sodium hydroxide (0.297 mL) at room temperature. The resulting solution was concentrated under reduced pressure to give the title compound (156 mg).

### Example E-04

### (S)-5-(2-chloro-4-methyl-phenylcarbamoyl)-4-(4-cyclopropyl-3′-isobutyl-[5,5′]biisoxazolyl-3-yl)-pentanoic acid

### E-04-1

### tert-Butyl (S)-6-benzyloxy-3-(4-cyclopropyl-5-ethynyl-isoxazol-3-yl)-hexanoate

Trimethylsilyldiazomethane (2 M in hexane) (0.529 mL) and THF (2.0 mL) were mixed. n-Butyllithium (2.6 M in hexane) (0.393 mL) was added dropwise to the mixture at −78° C. The reaction mixture was stirred for 30 minutes at −78° C. A solution of tert-butyl (S)-6-benzyloxy-3-(4-cyclopropyl-5-formyl-isoxazol-3-yl)-hexanoate (350 mg) which is obtained in a similar way to E-49-2 in THF (2.0 mL) was added dropwise to the reaction mixture at −78° C. The reaction mixture was stirred for 5 min at −78° C., and then stirred for 1 hr at 0° C. To the reaction mixture was added aqueous 1 M hydrochloric acid at ice temperature. Ethyl acetate was added to the mixture. The organic layer was separated, and washed with water and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/15) to give the title compound (157 mg).

### E-04-2

### 3-Methyl-butylaldehyde oxime

Hydroxylammonium chloride (605 mg), THF (7.0 mL), water (7.0 mL) were mixed. Aqueous 4 M sodium hydroxide (2.18 mL) was added dropwise to the mixture at ice temperature. A mixture of 3-methyl-butylaldehyde (500 mg) and ethanol (20 mL) was added dropwise to the reaction mixture at ice temperature. The mixture was stirred at room temperature overnight. The pH of the reaction mixture was adjusted with aqueous 1 M hydrochloric acid to 6. To the mixture was added diethyl ether. The organic layer was separated, and washed with brine, and then dried over sodium sulfate. The sodium sulfate was filtered off, and the filtrate was concentrated under reduced pressure to give the title compound as a crude product.

### E-04-3

### 1-Chloro-3-methyl-butylaldehyde oxime

3-Methyl-butylaldehyde oxime (48 mg) and DMF (1.0 mL) were mixed. To the mixture was added N-chlorosuccinimide (69 mg) at room temperature. The reaction mixture was stirred for 1 hr at room temperature. To the reaction mixture were added toluene and water. The organic layer was separated, and washed with water and aqueous saturated sodium chloride in this order, and the solution was directly used in the next step.

### E-04-4

### tert-Butyl (S)-6-benzyloxy-3-(4-cyclopropyl-3′-isobutyl-[5,5′]biisoxazolyl-3-yl)-hexanoate

A solution of 1-chloro-3-methyl-butylaldehyde oxime which was obtained in E-04-3 in toluene (equivalent to 0.467 mmol), potassium carbonate (0.078 g), water (3.0 mL), and tert-butyl (S)-6-benzyloxy-3-(4-cyclopropyl-5-ethynyl-isoxazol-3-yl)-hexanoate which was obtained in E-04-1 (153 mg) were mixed. The mixture was stirred for 3.5 hrs at 100° C. To the reaction mixture were added ethyl acetate and water. The organic layer was separated, and washed with water and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/15) to give the title compound (162 mg) as a crude product.

### E-04-5

### (S)-6-Benzyloxy-3-(4-cyclopropyl-3′-isobutyl-[5,5′]biisoxazolyl-3-yl)-hexanoic acid

tert-Butyl (S)-6-benzyloxy-3-(4-cyclopropyl-3′-isobutyl-[5,5′]biisoxazolyl-3-yl)-hexanoate (162 mg) and toluene (1.3 mL) were mixed. To the mixture was added trifluoroacetic acid (0.33 mL) at ice temperature. The reaction mixture was stirred at room temperature overnight, and concentrated under reduced pressure. The resulting residue was azeotroped with toluene to give the title compound (151 mg) as a crude product.

### E-04-6

### (S)-6-Benzyloxy-3-(4-cyclopropyl-3′-isobutyl-[5,5′]biisoxazolyl-3-yl)-hexanoic acid (2-chloro-4-methyl-phenyl)amide

(S)-6-Benzyloxy-3-(4-cyclopropyl-3′-isobutyl-[5,5′]biisoxazolyl-3-yl)-hexanoic acid (151 mg), 2-chloro-4-methyl-phenylamine (0.050 mL), and DMF (1.5 mL) were mixed. To the mixture were added diisopropylethylamine (0.089 mL) and HATU (156 mg) at ice temperature. The resulting mixture was stirred overnight at room temperature. To the reaction mixture were added ethyl acetate and water. The organic layer was separated, and washed with water and brine in this order. The organic layer was dried over sodium sulfate. The sodium sulfate was filtered off, and the filtrate was concentrated under reduced pressure. The resulting residue was purified by silica-gel column chromatography to give the title compound (161 mg).

### E-04-7

### (S)-3-(4-cyclopropyl-3′-isobutyl-[5,5′]biisoxazolyl-3-yl)-6-hydroxy-hexanoic acid (2-chloro-4-methyl-phenyl)amide

(S)-6-benzyloxy-3-(4-cyclopropyl-3′-isobutyl-[5,5′]biisoxazolyl-3-yl)-hexanoic acid (2-chloro-4-methyl-phenyl)amide (160 mg) and dichloromethane (2.5 mL) were mixed. To the mixture was added boron tribromide (1 M in dichloromethane) (0.833 mL) at ice temperature. The cooling bath was removed, and the reaction mixture was stirred for 20 minutes. To the reaction mixture were added aqueous saturated sodium hydrogen carbonate, water, and ethyl acetate at ice temperature. The organic layer was separated, and washed with aqueous saturated sodium hydrogen carbonate and brine. The organic layer was dried over sodium sulfate. The sodium sulfate was filtered off, and the filtrate was concentrated under reduced pressure.

The resulting residue was purified by silica-gel column chromatography (Eluent: ethyl acetate/hexane=1/1) to give a crude product (109 mg). The crude product was purified by preparative chromatography (Eluent: acetone/hexane 1/2) to give the title compound (65.7 mg).

### E-04-8

### (S)-5-(2-chloro-4-methyl-phenylcarbamoyl)-4-(4-cyclopropyl-3′-isobutyl-[5,5′]biisoxazolyl-3-yl)-pentanoic acid

(S)-3-(4-cyclopropyl-3′-isobutyl-[5,5′]biisoxazolyl-3-yl)-6-hydroxy-hexanoic acid (2-chloro-4-methyl-phenyl)amide (65.7 mg), acetonitrile (0.4 mL) and 1.0 M phosphate buffer (pH=6.8) (0.4 mL) were mixed. To the mixture were added 2,2,6,6-tetramethyl-1-piperidinyloxy radical (TEMPO) (2.1 mg), sodium chlorite (31 mg), aqueous sodium hypochlorite (Wako Pure Chemical Industries, 0.027 mL) at ice temperature. The reaction mixture was stirred for 1 hr at room temperature, and then aqueous 20 w/v % sodium sulfite and ethyl acetate were added to the mixture at ice temperature. The organic layer was separated, and washed with water and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/1) to give the title compound (68.8 mg).

### E-04-9

### Sodium (S)-5-(2-chloro-4-methyl-phenylcarbamoyl)-4-(4-cyclopropyl-3′-isobutyl-[5,5′]biisoxazolyl-3-yl)-pentanoate

(S)-5-(2-chloro-4-methyl-phenylcarbamoyl)-4-(4-cyclopropyl-3′-isobutyl-[5,5′]biisoxazolyl-3-yl)-pentanoic acid (68.0 mg) and ethanol (0.7 mL) were mixed. To the mixture was added aqueous 1 M sodium hydroxide (0.135 mL) at room temperature. The resulting solution was concentrated under reduced pressure to give the title compound (65.3 mg).

### Example E-62

### Sodium (S)-5-(4-chloro-2-fluoro-phenylcarbamoyl)-4-[5-(1,1-difluoro-2,2-dimethylpropyl)-4′-difluoromethyl-[3,5′]biisoxazolyl-3′-yl]-pentanoate

### E-62-1

### tert-Butyl (S)-6-benzyloxy-3-[5-(1-hydroxy-2,2-dimethyl-propyl)-4′-iodo-[3,5′]biisoxazolyl-3′-yl]-hexanoate

4,4-dimethyl-1-pentyn-3-ol which is contained in a similar way to E-49-5 (700 mg), aqueous 2.0 M potassium carbonate (2.02 mL), and toluene (5.0 mL) were mixed. The mixture was heated to 110° C. A solution of tert-butyl (S)-6-benzyloxy-3-[5-(chloro-hydroxyimino-methyl)-4-iodo-isoxazol-3-yl]-hexanoate which is contained in a similar way to E-58-15 (equivalent to 3.85 mmol) in toluene was added dropwise to the mixture. The resulting mixture was stirred for 30 minutes. To the reaction mixture was added toluene. The organic layer was separated, washed with water, and concentrated under reduced pressure. The resulting residue was purified by silica-gel column chromatography (Eluent: ethyl acetate/hexane=1/3) to give the title compound (2.51 g).

### E-62-2

### tert-Butyl (S)-6-benzyloxy-3-[5-(2,2-dimethyl-propionyl)-4′-iodo-[3,5′]biisoxazolyl-3′-yl]-hexanoate

tert-Butyl (S)-6-benzyloxy-3-[5-(1-hydroxy-2,2-dimethyl-propyl)-4′-iodo-[3,5′]biisoxazolyl-3′-yl]-hexanoate (1.50 g) and chloroform (15 mL) were mixed. To the mixture was added Dess-Martin periodinane (1.23 g) at room temperature. The reaction mixture was stirred for 1 hr at room temperature. To the mixture was added aqueous sodium carbonate and aqueous saturated sodium hydrogen carbonate at ice temperature. To the reaction mixture was added ethyl acetate. The resulting mixture was stirred for minutes at room temperature. The organic layer was separated, and washed with aqueous saturated sodium hydrogen carbonate and brine in this order. The organic layer was dried over sodium sulfate. The sodium sulfate was filtered off, and the filtrate was concentrated under reduced pressure. The resulting residue was purified by silica-gel column chromatography (Fluent: ethyl acetate/hexane=1/7.5) to give the title compound (1.46 g).

### E-62-3

### tert-Butyl (S)-6-benzyloxy-3-[5-(2,2-dimethyl-propionyl)-4′-vinyl-[3,5′]biisoxazolyl-3′-yl]-hexanoate

tert-Butyl (S)-6-benzyloxy-3-[5′-(1,1-difluoro-2,2-dimethyl-propyl)-4-iodo-[3,3′]biisoxazolyl-5-yl]-hexanoate (800 mg), 4,4,5,5-tetramethyl-2-vinyl-[1,3,2]dioxaborolane (300 mg), cesium fluoride (880 mg), and N-methylpyrrolidone (5.5 mL) were mixed. To the mixture was added PdCl₂ (dppf).CH₂Cl₂ (52.5 mg) under argon atmosphere. The reaction mixture was stirred at 90° C. overnight under argon atmosphere. To the reaction mixture were added ethyl acetate and water. The organic layer was separated, and washed with water. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/6) to give the title compound (257 mg).

### E-62-4

### tert-Butyl (S)-6-benzyloxy-3-[5-(2,2-dimethyl-propionyl)-4′-(2-phenyl[1,3,2]dioxaborolane-4-yl)-[3,5′]biisoxazolyl-3′-yl]-hexanoate

tert-Butyl (S)-6-benzyloxy-3-[5-(2,2-dimethyl-propionyl)-4′-vinyl-[3,5′]biisoxazolyl-3′-yl]-hexanoate (257 mg) and dichloromethane (2.0 mL) were mixed. To the mixture were added phenylboric acid (120 mg), N-methylmorpholine N-oxide (115 mg), and microencapsulated osmium tetroxide (10 w/w %, 62 mg) at room temperature. The resulting mixture was stirred for 2 hrs at room temperature. To the reaction mixture were added aqueous saturated sodium dithionite and chloroform at room temperature. The organic layer was separated, and washed with brine, and then dried over sodium sulfate. The sodium sulfate was filtered off, and the filtrate was concentrated under reduced pressure. The resulting residue was purified by silica-gel column chromatography (Eluent: ethyl acetate/hexane=1/2) to give the title compound (282 mg).

### E-62-5

### tert-Butyl (S)-6-benzyloxy-3-[5-(2,2-dimethyl-propionyl)-4′-formyl-[3,5′]biisoxazolyl-3′-yl]-hexanoate

tert-Butyl (S)-6-benzyloxy-3-[5-(2,2-dimethyl-propionyl)-4′-(2-phenyl[1,3,2]dioxaborolane-4-yl)-[3,5′]biisoxazolyl-3′-yl]-hexanoate (282 mg), THF (30 mL), and water (3.0 mL) were mixed. To the mixture was added sodium periodate (282 mg) at room temperature. The reaction mixture was stirred for 30 minutes at 50° C. To the reaction mixture were added diethyl ether and water. The organic layer was separated, and dried over sodium sulfate. The sodium sulfate was filtered off, and the filtrate was concentrated under reduced pressure. The resulting residue was purified by silica-gel column chromatography (Eluent: ethyl acetate/hexane=1/4) to give the title compound (145 mg).

### E-62-6

### tert-Butyl (S)-6-benzyloxy-3-[5-(1,1-difluoro-2,2-dimethylpropyl)-4′-difluoromethyl-[3,5′]biisoxazolyl-3′-yl]-hexanoate

tert-Butyl (S)-6-benzyloxy-3-[5-(2,2-dimethyl-propionyl)-4′-formyl-[3,5′]biisoxazolyl-3′-yl]-hexanoate (145 mg) and bis(2-methoxyethyl)aminosulfur trifluoride (0.306 mL) were mixed. The mixture was stirred for 12 hrs at room temperature. The reaction mixture was stirred for 1 hr at 80° C. To the mixture was added chloroform at room temperature. The reaction mixture was added to aqueous saturated sodium hydrogen carbonate at ice temperature. To the reaction mixture was added chloroform. The organic layer was separated, and washed with brine, and then dried over sodium sulfate. The sodium sulfate was filtered off, and the filtrate was concentrated under reduced pressure. The resulting residue was purified by silica-gel column chromatography (Eluent: ethyl acetate/hexane=1/6) to give the title compound (125 mg).

### E-62-7

### (S)-6-benzyloxy-3-[5-(1,1-difluoro-2,2-dimethylpropyl)-4′-difluoromethyl-[3,5′]biisoxazolyl-3′-yl]-hexanoic acid

tert-Butyl (S)-6-benzyloxy-3-[5-(1,1-difluoro-2,2-dimethylpropyl)-4′-difluoromethyl-[3,5′]biisoxazolyl-3′-yl]-hexanoate (125 mg) and toluene (0.60 mL) were mixed. To the mixture was added trifluoroacetic acid (0.60 mL) at ice temperature. The reaction mixture was stirred for 2 hrs at room temperature, and concentrated under reduced pressure. The resulting residue was azeotroped with toluene to give the title compound (122 mg) as a crude product.

### E-62-8

### (S)-6-benzyloxy-3-[5-(1,1-difluoro-2,2-dimethylpropyl)-4′-difluoromethyl-[3,5′]biisoxazolyl-3′-yl]-hexanoic acid (2-chloro-4-methyl-phenyl)amide

(S)-6-benzyloxy-3-[5-(1,1-difluoro-2,2-dimethylpropyl)-4′-difluoromethyl-[3,5′]biisoxazolyl-3′-yl]-hexanoic acid (122 mg), 4-chloro-2-fluoro-phenylamine (42 mg), and DMF (3.0 mL) were mixed. To the mixture were added diisopropylethylamine (0.083 mL), and HATU (109 mg) at room temperature. The resulting mixture was stirred overnight at room temperature. To the reaction mixture was added aqueous saturated sodium bicarbonate at ice temperature. The resulting was extracted with ethyl acetate. The organic layer was washed with water and brine in this order. The organic layer was dried over sodium sulfate. The sodium sulfate was filtered off, and the filtrate was concentrated under reduced pressure. The resulting residue was purified by silica-gel column chromatography (Eluent: ethyl acetate/hexane=1/4) to give the title compound (146 mg).

### E-62-9

### (S)-3-[5-(1,1-difluoro-2,2-dimethylpropyl)-4′-difluoromethyl-[3,5′]biisoxazolyl-3′-yl]-6-hydroxy-hexanoic acid (2-chloro-4-methyl-phenyl)amide

(S)-6-benzyloxy-3-[5-(1,1-difluoro-2,2-dimethylpropyl)-4′-difluoromethyl-[3,5′]biisoxazolyl-3′-yl]-hexanoic acid (2-chloro-4-methyl-phenyl)amide (146 mg) and dichloromethane (1.5 mL) were mixed. To the mixture was added boron tribromide (1 M in dichloromethane) (0.680 mL) at −78° C. The reaction mixture was stirred for 15 min at −78° C., and then stirred for 15 min at 0° C. To the reaction mixture were added aqueous saturated sodium hydrogen carbonate and ethyl acetate at ice temperature. The organic layer was separated, and washed with brine. The organic layer was dried over sodium sulfate. The sodium sulfate was filtered off, and the filtrate was concentrated under reduced pressure. The resulting residue was purified by silica-gel column chromatography (Eluent: ethyl acetate/hexane=2/3) to give the title compound (84.8 mg).

### E-62-10

### (S)-5-(4-chloro-2-fluoro-phenylcarbamoyl)-4-[5-(1,1-difluoro-2,2-dimethylpropyl)-4′-difluoromethyl-[3,5′]biisoxazolyl-3′-yl]-pentanoic acid

(S)-3-[5-(1,1-difluoro-2,2-dimethylpropyl)-4′-difluoromethyl-[3,5′]biisoxazolyl-3′-yl]-6-hydroxy-hexanoic acid (2-chloro-4-methyl-phenyl)amide (84.8 mg), acetonitrile (0.43 mL), and 1.0 M phosphate buffer (pH=6.8) (0.17 mL) were mixed. To the mixture were added 2,2,6,6-tetramethyl-1-piperidinyloxy radical (TEMPO) (1.3 mg), sodium chlorite (40 mg), and aqueous sodium hypochlorite (Wako Pure Chemical Industries, 0.030 mL) at room temperature. The reaction mixture was stirred for 1.5 hrs at room temperature. To the reaction mixture were added aqueous sodium sulfite and aqueous sodium hydrogen sulfate at ice temperature. Ethyl acetate was added to the reaction mixture. The organic layer was separated, and washed with brine, and then dried over sodium sulfate. The sodium sulfate was filtered off, and the filtrate was concentrated under reduced pressure. The resulting residue was purified by preparative chromatography (Eluent: methanol/chloroform=1/15) to give the title compound (67.0 mg).

### E-62-11

### Sodium (S)-5-(4-chloro-2-fluoro-phenylcarbamoyl)-4-[5-(1,1-difluoro-2,2-dimethylpropyl)-4′-difluoromethyl-[3,5′]biisoxazolyl-3′-yl]-pentanoate

(S)-5-(4-chloro-2-fluoro-phenylcarbamoyl)-4-[5-(1,1-difluoro-2,2-dimethylpropyl)-4′-difluoromethyl-[3,5′]biisoxazolyl-3′-yl]-pentanoic acid (67.0 mg) and ethanol (0.119 mL) were mixed. To the mixture was added aqueous 1 M sodium hydroxide (0.119 mL) at room temperature. The resulting solution was concentrated under reduced pressure to give the title compound (70.3 mg).

### Production Example of Example E-08

### Production Example of Example E-15

### Production Example of Example E-18

### Production Example of Example E-14

### Production Example of Example E-11

### Example F-499

### 4-(2-chloro-5-methylphenylcarbamoyl)-3-[4-cyclopropyl-5-(3-isobutylcyclobutyl) isoxazol-3-yl]butanoic acid

### F-499-1

### 4-Methyl-1-piperidine-1-ylpentan-1-one

4-Methyl valerate (238 g) and DMF (833 mL) were mixed. To the mixture were added piperidine (233 mL), HOBt.H₂O (361 g), and WSC.HCl (452 g) at ice temperature. The resulting mixture was stirred overnight at room temperature. To the reaction mixture was added water (1000 mL) at ice temperature. The resulting mixture was extracted with toluene (500 mL×2). The organic layer was washed with aqueous 10 w/v % sodium carbonate (500 mL+300 mL) and water (500 mL×2) in this order, and concentrated under reduced pressure to give the title compound (414.29 g) as a crude product.

### F-499-2

### 1-(4-Methyl-1-pentenyl)piperidine

4-Methyl-1-piperidine-1-ylpentan-1-one (372.4 g) and toluene (1000 mL) were mixed. To the mixture was added (Ph₃P)IrCl(CO) (633 mg). 1,1,3,3-Tetramethyldisiloxane (627 mL) was added dropwise to the mixture under cooling with water, and then the resulting mixture at was stirred for 2 hrs room temperature. The reaction mixture was concentrated under reduced pressure to give the title compound (844 g) as a crude product.

### F-499-3

### Ethyl 3-isobutyl-2-piperidine-1-ylcyclobutanecarboxylate

1-(4-methyl-1-pentenyl)piperidine (844 g) and acetonitrile (70 mL) were mixed. To the mixture were added ethyl acrylate (443 mL) and hydroquinone (447 mg). The resulting mixture was stirred overnight at 95° C. The reaction mixture was concentrated under reduced pressure to give the title compound (994.08 g) as a crude product.

### F-499-4

### 3-Isobutyl-1-cyclobutenecarboxylic acid

Ethyl 3-isobutyl-2-piperidine-1-ylcyclobutanecarboxylate (994 g) and methyl p-toluenesulfonate (337 mL) were mixed. The mixture was stirred for 2 hrs at 110° C. To the reaction mixture was added water (1100 mL). The resulting mixture was washed with a mixture of tert-butyl methyl ether:hexane=1:1 (600 mL) and hexane (600 mL) in this order. To the aqueous layer was added potassium hydroxide (503 g) at ice temperature, and the mixture was stirred for hrs at 95° C. The reaction mixture was washed with diethyl ether (500 mL), a mixture of diethyl ether:hexane=1:1 (500 mL) in this order. To the aqueous layer was added concentrated hydrochloric acid (672 mL) at ice temperature, and the mixture was extracted with ethyl acetate (1 L×2). The organic layer was washed with water (500 mL×2) and brine (500 mL) in this order. The organic layer was dried over sodium sulfate. The sodium sulfate was filtered off, and the filtrate was concentrated under reduced pressure to give the title compound (240 g) as a crude product.

### F-499-5

### 3-Isobutylcyclobutanecarboxylic acid

3-Isobutyl-1-cyclobutenecarboxylic acid (188 g) and tetrahydrofuran (2000 mL) were mixed. To the mixture was added 5 w/w % rhodium on activated carbon (5.64 g). The mixture was stirred for 7 hrs at room temperature under hydrogen atmosphere (1 atm). The 5 w/w % rhodium on activated carbon was filtered off and the filtrate was concentrated under reduced pressure to give the title compound (134.06 g) as a crude product.

### F-499-6

### 3-Isobutyl-cyclobutanecarboxylic acid cyclopropylamide

To a solution of 3-isobutyl-cyclobutanecarboxylic acid (20.5 g) and HOBt.H₂O (24 g) in acetonitrile (300 mL) were added cyclopropylamine (10 mL) and WSC.HCl (30 g) at ice temperature. The resulting mixture was stirred overnight at room temperature. The reaction mixture was concentrated under reduced pressure, and then water and ethyl acetate were added thereto. The organic layer was separated, and washed with aqueous 10% citric acid, water, aqueous saturated sodium hydrogen carbonate, water, and brine in this order. The organic layer was dried over sodium sulfate. The sodium sulfate was filtered off, and the filtrate was concentrated under reduced pressure to give the title compound (28 g) as a crude product.

### F-499-7

### 3-Isobutyl-cyclobutanecarbothioic acid cyclopropylamide

To a solution of 3-isobutyl-cyclobutanecarboxylic acid cyclopropylamide (2.93 g) in tetrahydrofuran (25 mL) was added Lawesson's reagent (3.64 g) at room temperature. The mixture was stirred for 2 hrs at room temperature, and then the reaction mixture was concentrated under reduced pressure, and the resulting residue was purified by silica-gel column chromatography (A mixture of ethyl acetate and hexane was used as an eluent. Eluent: ethyl acetate hexane=1:7) to give the title compound (2.75 g).

### F-499-8

### N-Cyclopropyl-3-isobutyl-cyclobutanecarboximidethioic acid methyl ester hydrogen iodide salt

Methyl iodide (3.2 mL) was added dropwise to a solution of 3-isobutyl-cyclobutanecarbothioic acid cyclopropylamide (2.1 g) in acetonitrile (10 mL) at room temperature. The mixture was stirred for 2 hrs at room temperature, and then concentrated under reduced pressure. The residue was azeotroped with chloroform to give the title compound (3.8 g) as a crude product.

### F-499-9

### tert-Butyl 4-oxo-4-(2-oxo-oxazolidine-3-yl)butyrate

To a solution of 2-oxazolidinone (2.5 g) and mono-tert-butyl succinate (5.0 g) in chloroform (30 mL) were added WSC.HCl (6.62 g) and DMAP (350 mg) at room temperature. The mixture was stirred overnight at room temperature. The reaction mixture was concentrated under reduced pressure, and then ethyl acetate was added. The organic layer was washed with aqueous saturated sodium hydrogen carbonate, water, and brine in this order. The organic layer was dried over sodium sulfate. The sodium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=4:1, followed by 1:1) to give the title compound (5.92 g).

### F-499-10

### tert-Butyl 3-(2-oxo-oxazolidine-3-carbonyl)-5-hexanoate

A solution of 1 M NaH MDS in tetrahydrofuran (4.8 mL) was added dropwise to a solution of tert-butyl 4-oxo-4-(2-oxo-oxazolidine-3-yl)butyrate (2.57 g) in tetrahydrofuran (45 mL) over 5 minutes at −78° C. The mixture was stirred for 1 hr at −78° C. Allyl iodide (4.8 mL) was added dropwise to the mixture over 3 minutes. The reaction temperature was gradually risen. The mixture was stirred overnight at room temperature, and then aqueous saturated ammonium chloride and chloroform were added thereto. The organic layer was separated, and washed with aqueous 1 N HCl, aqueous 1 N NaGH, and brine in this order, and then dried with magnesium sulfate. The magnesium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:3) to give the title compound (1.46 g).

### F-499-11

### 4-tert-Butyl 2-allyl-succinate

To a reaction solution of tert-butyl 3-(2-oxo-oxazolidine-3-carbonyl)-5-hexanoate (1.46 g) in tetrahydrofuran (20 mL) and H₂O (5.0 mL) were added lithium hydroxide monohydrate (350 mg) and aqueous 30% hydrogen peroxide (2.34 mL) at ice temperature. The reaction mixture was stirred for 1 hr, and then, to the mixture were added water, aqueous 5% Na₂SO₃, and aqueous 10% citric acid. The resulting mixture was extracted with chloroform, and dried with magnesium sulfate. The magnesium sulfate was filtered off, and the filtrate was concentrated under reduced pressure to give the title compound (899 mg) as a crude product.

### F-499-12

### tert-Butyl 3-hydrazinocarbonyl-5-hexanoate

4-tert-Butyl 2-allyl-succinate (899 mg) and acetonitrile (9.0 mL) were mixed. To the mixture were added HOBt.H₂O (772 mg) and WSC.HCl (965 mg) at room temperature. The mixture was stirred for 1 hr and 30 minutes. The reaction mixture was added dropwise to a solution of NH₂NH₂.H₂O (0.41 mL), cyclohexene (0.1 mL), and acetonitrile (4.0 mL) with washing with acetonitrile (3.0 mL) ice temperature. Then, the reaction mixture was stirred for 40 minutes at ice temperature. To the mixture were added water and ethyl acetate. The organic layer was separated, and washed with aqueous saturated sodium hydrogen carbonate and brine in this order. The organic layer was dried over magnesium sulfate. The magnesium sulfate was filtered off, and the filtrate was concentrated under reduced pressure to give the title compound (844 mg) as a crude product.

### F-499-13

### tert-Butyl (3-[4-cyclopropyl-5-(3-isobutyl-cyclobutyl)-4H-[1,2,4]triazol-3-yl]-hex-5-enoate

tert-Butyl 3-hydrazinocarbonyl-5-hexanoate (228 mg), N-cyclopropyl-3-isobutyl-cyclobutanecarboximidethioic acid methyl ester hydrogen iodide salt (1.04 g), and ethanol (10 mL) were mixed. The mixture was stirred for 7 hrs at room temperature. The reaction mixture was concentrated under reduced pressure. To the mixture was added aqueous saturated sodium hydrogen carbonate. The mixture was extracted with ethyl acetate (twice). The organic layer was washed with brine, and was dried over sodium sulfate. The sodium sulfate was filtered off, and the filtrate was concentrated under reduced pressure. To the resulting residue were added 1,4-dioxane (5.0 mL) and acetic acid (0.5 mL). The resulting mixture was stirred overnight at 90° C. The reaction mixture was cooled to room temperature. Then the pH of the reaction mixture was adjusted with saturated sodium hydrogen carbonate to 8. To the mixture were added ethyl acetate and water. The organic layer was separated, and washed with brine, and then dried over sodium sulfate. The sodium sulfate was filtered off, and the filtrate was concentrated under reduced pressure. The resulting residue was purified by silica-gel column chromatography (A mixture of methanol and chloroform was used as an eluent. Eluent: methanol:chloroform=2:100) to give the title compound (703 mg).

### F-499-14

### Mono-tert-butyl 3-[4-cyclopropyl-5-(3-isobutyl-cyclobutyl)-4H-[1,2,4]triazol-3-yl]-pentanedioate

tert-Butyl 3-[4-cyclopropyl-5-(3-isobutyl-cyclobutyl)-4H-[1,2,4]triazol-3-yl]-hex-5-enoate (301 mg), acetonitrile (3.0), carbon tetrachloride (3.0 mL), and H₂O (3.0 mL) were mixed. To the mixture were added NaIO₄ (831 mg) and RuCl₃.H₂O (18 mg) at ice temperature. The reaction mixture stirred for 4 hrs at room temperature. And then, isopropyl alcohol (3.0 mL) and Buffer were added. Ethyl acetate was added to the mixture. The organic layer was separated, and washed with aqueous 10% NaS₂O₄ and brine in this order. The organic layer was dried over magnesium sulfate. The magnesium sulfate was filtered off, and the filtrate was concentrated under reduced pressure. The resulting residue was purified by silica-gel column chromatography (A mixture of methanol and chloroform was used as an eluent. Eluent: methanol:chloroform=4:100) to give the title compound (239 mg).

### F-499-15

### tert-Butyl (3-[4-cyclopropyl-5-(3-isobutyl-cyclobutyl)-4H-[1,2,4]triazol-3-yl]-4-(2,4-dimethyl-phenylcarbamoyl)-butyrate

Mono-tert-butyl 3-[4-cyclopropyl-5-(3-isobutyl-cyclobutyl)-4H-[1,2,4]triazol-3-yl]-pentanedioate (228 mg) and DMF (2.0 mL) were mixed. To the mixture were added HOBt.H₂O (103 mg), WSC.HCl (130 mg), and 2,4-dimethylaniline (0.084 mL) at room temperature. The mixture was stirred for 6 hrs and 30 minutes. To the reaction mixture were added water and ethyl acetate. The organic layer was separated, and washed with aqueous saturated sodium hydrogen carbonate. The organic layer was dried over sodium sulfate. The sodium sulfate was filtered off, and the filtrate was concentrated under reduced pressure. The resulting residue was purified by silica-gel column chromatography (A mixture of methanol and chloroform was used as an eluent. Eluent: methanol:chloroform=3:100) to give the title compound (294 mg). A part thereof was purified by preparative chromatography (A mixture of methanol and chloroform was used as an eluent. Eluent: methanol:chloroform=1:10) to give the title compound (9.6 mg).

### F-499-16

### 3-[4-Cyclopropyl-5-(3-isobutyl-cyclobutyl)-4H-[1,2,4]triazol-3-yl]-4-(2,4-dimethyl-phenylcarbamoyl)-butyl

tert-Butyl 3-[4-cyclopropyl-5-(3-isobutyl-cyclobutyl)-4H-[1,2,4]triazol-3-yl]-4-(2,4-dimethyl-phenylcarbamoyl)-butyrate (271.5 mg) and chloroform (7.0 mL) were mixed. To the mixture was added trifluoroacetic acid (1.4 mL) at room temperature. The reaction mixture was stirred for 2 hrs and 40 min at room temperature, and stirred for 1 hr and 30 minutes at 50° C. Then the mixture was concentrated under reduced pressure, and the residue was azeotroped with toluene (three times). A part of the resulting residue was purified by preparative chromatography (A mixture of methanol and chloroform was used as an eluent. Eluent: methanol:chloroform=1:10) to give the title compound (6.8 mg).

### Example F-644

### (S)-3-[4-cyclopropyl-5-(3-isobutyl-cyclobutyl)-isoxazol-3-yl]-5-(1H-tetrazole-5-yl)-valeric acid (2,4-dimethyl-phenyl)-amide

### F-644-1

### 3-Isobutylcyclobutanecarboxylic acid methoxymethylamide

3-isobutylcyclobutanecarboxylic acid which was obtained in F-499-5 (62.7 g) and DMF (500 mL) were mixed. To the mixture were added N,O-dimethylhydroxylamine hydrochloride salt (46.9 g), triethylamine (83.9 mL), HOBt.H₂O (73.8 g), and WSC.HCl (92.3 g). The resulting mixture was stirred overnight at room temperature. To the reaction mixture was added water (500 mL). The resulting mixture was extracted with a mixture of ethyl acetate:hexane=1:1 (250 mL×2). The organic layer was washed with water (250 mL), aqueous 10 w/v % sodium carbonate (250 mL), water (250 mL), 1 N hydrochloric acid (500 mL), water, aqueous saturated sodium bicarbonate (250 mL), and brine (250 mL) in this order. The organic layer was dried over magnesium sulfate. The magnesium sulfate was filtered off, and the filtrate was concentrated under reduced pressure to give the title compound (87.5 g) as a crude product.

### F-644-2

### 3-Isobutylcyclobutanecarbaldehyde

Diisobutylaluminium hydride (1.0 M in methylene chloride) (473.2 mL) was added dropwise to a solution of 3-isobutylcyclobutanecarboxylic acid methoxymethylamide (77 g) in methylene chloride (235 mL) at −78° C. The mixture was stirred for 2 hrs at −78° C. 1.5 M sulfuric acid (630 mL) was added dropwise to the mixture. The aqueous layer was separated, and extracted with methylene chloride. The combined organic layer was washed with 1.5 M sulfuric acid, water, and brine in this order. The organic layer was dried over magnesium sulfate. The magnesium sulfate was filtered off. The filtrate comprising the title compound was directly used in the next step.

### F-644-3

### 1-(2,2-Dibromovinyl)-3-isobutylcyclobutane

A solution of triphenylphosphine (266 g) in methylene chloride (350 mL) was added dropwise to a solution of carbon tetrabromide (168 g) in methylene chloride (252 mL) at ice temperature. The mixture was stirred for 20 min at ice temperature. Then, a solution of 3-isobutylcyclobutanecarbaldehyde in methylene chloride was added dropwise to the mixture at ice temperature. The mixture was stirred for 20 min at ice temperature. Aqueous 10 w/v % sodium carbonate (1 L) was added dropwise to the mixture. The aqueous layer was separated, and was extracted with methylene chloride (200 mL×2), then the organic layer was washed with water and brine in this order. The organic layer was dried over magnesium sulfate. The magnesium sulfate was filtered off, and the filtrate was concentrated under reduced pressure. To the resulting residue were added a mixture of hexane:chloroform=1:1 (750 mL), silica gel (750 mL), hexane (900 mL) in this order. The mixture was filtered and the filtrate was concentrated under reduced pressure. To the residue was added hexane (500 mL), and the mixture was filtered. The filtrate was concentrated under reduced pressure, and the resulting residue was purified by silica-gel column chromatography (Hexane was used as an eluent.) to give the title compound (76.21 g).

### F-644-4

### 5-(tert-Butyl-diphenyl-silanyloxy)pentanoic acid

δ-Valerolactone (6.23 g) and methanol (200 mL) were mixed. To the mixture was added concentrated sulfuric acid (8 drops). The mixture was heated to reflux overnight. To the reaction mixture was added imidazole (600 mg) at room temperature, and then the mixture was concentrated under reduced pressure. To the resulting residue were added DMF (40 mL), imidazole (5.08 g), and tert-butylchlorodiphenylsilane (19.4 mL). The reaction mixture was stirred for 3 hrs at room temperature, and then to the mixture were added water, diethyl ether, and hexane at ice temperature. The mixture was extracted with diethyl ether, and then the resulting organic layer was washed with aqueous saturated ammonium chloride, and dried with magnesium sulfate. The magnesium sulfate was filtered off, and the filtrate was concentrated under reduced pressure. To the resulting residue were added methanol (50 mL) and aqueous 2N sodium hydroxide (34.2 mL). The reaction mixture was stirred for 5 hrs at room temperature, and concentrated under reduced pressure. The residue was extracted with ethyl acetate. The organic layer was washed with brine, and dried with magnesium sulfate. The magnesium sulfate was filtered off, and the filtrate was concentrated under reduced pressure to give the title compound (24.4 g) as a crude product.

### F-644-5

### (R)-4-Benzyl-3-[5-(tert-butyl-diphenyl-silanyloxy)pentanoyl]-oxazolidin-2-one

5-(tert-Butyl-diphenyl-silanyloxy)pentanoic acid (23.51 g), (R)-4-benzyl-2-oxazolidinone (11 g), and chloroform (200 ml) were mixed. To the mixture were added 4-dimethylaminopyridine (3.8 g) and WSC.HCl (12.5 g), and the mixture was stirred for 7.5 hrs at room temperature. The reaction mixture was concentrated under reduced pressure. To the resulting residue was added ethyl acetate. To the mixture were added 2 N hydrochloric acid and water, and the mixture was extracted with ethyl acetate. The organic layer was washed with 2 N hydrochloric acid and brine in this order. The organic layer was dried over magnesium sulfate. The magnesium sulfate was filtered off, and the filtrate was concentrated under reduced pressure, and the resulting residue was purified by silica-gel column chromatography (A mixture of ethyl acetate and hexane was used as an eluent. Eluent: ethyl acetate:hexane=1:10, followed by 1:6) to give the title compound (27.1031 g).

### F-644-6

### tert-Butyl (S)-3-((R)-4-benzyl-2-oxo-oxazolidine-3-carbonyl)-6-(tert-butyl-diphenyl-silanyloxy)-hexanoate

Sodium hexamethyldisilazide (1.9 M in THF) (30.8 mL) and THF (125 mL) were mixed. A solution of (R)-4-benzyl-3-[5-(tert-butyl-diphenyl-silanyloxy)pentanoyl]-oxazolidin-2-one (25.12 g) in THF was added dropwise to the mixture at −78° C. tert-Butyl bromoacetate (11.5 mL) was added dropwise to the reaction mixture at −66° C. The temperature of the reaction mixture was raised to −26° C. over 0.5 hrs. The mixture was stirred for 0.5 hrs at ice temperature. Aqueous saturated ammonium chloride was added dropwise to the reaction mixture. The mixture was extracted with ethyl acetate, and the resulting organic layer was washed with aqueous saturated ammonium chloride and brine in this order, and dried with magnesium sulfate. The magnesium sulfate was filtered off, and the filtrate was concentrated under reduced pressure, and the resulting residue was purified by silica-gel column chromatography (A mixture of ethyl acetate and hexane was used as an eluent. Eluent: ethyl acetate:hexane=1:50, 1:20, and 1:10 in this order) to give the title compound (26.04 g).

### F-644-7

### 4-tert-Butyl (S)-2-[3-(tert-butyl-diphenyl-silanyloxy)-propyl]-succinate

Lithium hydroxide monohydrate (2.79 g), THF (80 mL) and water (54 mL) were mixed. Aqueous 30 w/w % hydrogen peroxide (18.9 mL) was added dropwise to the mixture at ice temperature. The reaction mixture was stirred for 0.5 hrs at ice temperature. tert-Butyl (S)-3-((R)-4-benzyl-2-oxo-oxazolidine-3-carbonyl)-6-(tert-butyl-diphenyl-silanyloxy)-hexanoate (25.6 g) was added dropwise to the reaction mixture at ice temperature. The reaction mixture was stirred for 1.5 hrs, and then aqueous 10 w/v % sodium hydrogen sulfite (350 mL) was added dropwise to the mixture at ice temperature. The mixture was extracted with ethyl acetate, and the resulting organic layer was washed with aqueous sodium hydrogen sulfite, and citric acid aqueous in this order, and then dried with magnesium sulfate. The magnesium sulfate was filtered off, and the filtrate was concentrated under reduced pressure, and the resulting residue was purified by silica-gel column chromatography (A mixture of ethyl acetate and hexane was used as an eluent. Eluent: ethyl acetate:hexane=1:9, 1:8, 1:6, and 1:4 in this order) to give the title compound (17.13 g).

### F-644-8

### tert-Butyl (S)-6-(tart-butyl-diphenyl-silanyloxy)-3-(methoxy-methyl-carbamoyl)-hexanoate

4-tert-Butyl (S)-2-[3-(tert-butyl-diphenyl-silanyloxy)-propyl]-succinate (16.47 g), N,N-diisopropylethylamine (8.32 mL), N,O-dimethylhydroxylamine hydrochloride salt (4.1 g), and chloroform (170 mL) were mixed. To the mixture were added 4-(dimethylamino)pyridine (4.27 g), and WSC.HCl (7.38 g). The mixture was stirred for 2 hrs at room temperature. The reaction mixture was concentrated under reduced pressure, and the resulting residue was purified by silica-gel column chromatography (A mixture of ethyl acetate and hexane was used as an eluent. Eluent: ethyl acetate:hexane=1:100, 1:20, 1:10, 1:9, 1:8, and 1:4 in this order) to give the title compound (14.93 g).

### F-644-9

### tert-Butyl (S)-3-[3-(tert-butyl-diphenyl-silanyloxy)-propyl]-6-(3-isobutyl-cyclobutyl)-4-oxo-5-hexynoate

1-(2,2-Dibromovinyl)-3-isobutylcyclobutane which was obtained in F-644-3 (9.47 g) and tetrahydrofuran (100 mL) were mixed. n-Butyllithium (2.66 M in hexane) (25.8 mL) was added dropwise to the mixture at −78° C. The mixture was stirred at ice temperature. Then, a solution of tert-butyl (S)-6-(tert-butyl-diphenyl-silanyloxy)-3-(methoxy-methyl-carbamoyl)-hexanoate (11.52 g) in tetrahydrofuran (40 mL) was added dropwise to the mixture. The mixture was stirred for 0.5 hrs at ice temperature. To the reaction mixture was added aqueous saturated ammonium chloride, and the mixture was extracted with ethyl acetate. the resulting organic layer was washed with aqueous saturated ammonium chloride, and dried with magnesium sulfate. The magnesium sulfate was filtered off, and the filtrate was concentrated under reduced pressure, and the resulting residue was purified by silica-gel column chromatography (A mixture of ethyl acetate and hexane was used as an eluent. Eluent: ethyl acetate:hexane=1:30, 1:25, and 1:20 in this order) to give the title compound (11.75 g).

### F-644-10

### tert-Butyl (S)-3-[3-(tert-butyl-diphenyl-silanyloxy)-propyl]-6-(3-isobutyl-cyclobutyl)-4-(methoxyimino)-5-hexynoate

tert-Butyl (S)-3-[3-(tert-butyl-diphenyl-silanyloxy)-propyl]-6-(3-isobutyl-cyclobutyl)-4-oxo-5-hexynoate (11.57 g) and ethanol (100 mL) were mixed. To the mixture were added sodium carbonate (10.4 g), O-methylhydroxylammonium chloride (8.2 g) at ice temperature. The mixture was stirred for 1.5 hrs at 80° C. To the mixture was added water at room temperature, and the mixture was extracted with ethyl acetate, and the resulting organic layer was washed with brine, and dried with magnesium sulfate. The magnesium sulfate was filtered off, and the filtrate was concentrated under reduced pressure, and the resulting residue was purified by silica-gel column chromatography (A mixture of ethyl acetate and hexane was used as an eluent. Eluent: ethyl acetate:hexane=1:40, 1:30, 1:20, and 1:1 in this order) to give the title compound (11.9569 g).

### F-644-11

### tert-Butyl (S)-6-(tert-butyl-diphenyl-silanyloxy)-3-[4-iodo-5-(3-isobutyl-cyclobutyl)-isoxazol-3-yl]-hexanoate

tert-Butyl (S)-3-[3-(tert-butyl-diphenyl-silanyloxy)-propyl]-6-(3-isobutyl-cyclobutyl)-4-(methoxyimino)-5-hexynoate (2.38 g) and methylene chloride (38 mL) were mixed. To the mixture was added iodine monochloride (1 M in methylene chloride) (4.62 mL) at ice temperature. The mixture was stirred for 1 hr at room temperature, and then aqueous sodium thiosulfate was added dropwise to the reaction mixture. tert-Butyl (S)-3-[3-(tert-butyl-diphenyl-silanyloxy)-propyl]-6-(3-isobutyl-cyclobutyl)-4-(methoxyimino)-5-hexynoate (8.9679 g) was reacted in the same way as the above, and aqueous sodium thiosulfate was added dropwise to the reaction mixture. The two mixtures were combined, and the resulting mixture was extracted with chloroform, and the resulting organic layer was washed with brine and dried with magnesium sulfate. The magnesium sulfate was filtered off, and the filtrate was concentrated under reduced pressure, and the resulting residue was purified by silica-gel column chromatography (A mixture of diethyl ether and hexane was used as an eluent. Eluent: diethyl ether:hexane=1:100, 1:80, 1:60, and 1:40 in this order) to give the title compound (7.9839 g).

### F-644-12

### tert-Butyl (S)-6-(tert-butyl-diphenyl-silanyloxy)-3-[4-cyclopropyl-5-(3-isobutyl-cyclobutyl)-isoxazol-3-yl]-hexanoate

tert-Butyl (S)-6-(tert-butyl-diphenyl-silanyloxy)-3-[4-iodo-5-(3-isobutyl-cyclobutyl)-isoxazol-3-yl]-hexanoate (7.8643 g), 2-cyclopropyl-4,4,5,5-tetramethyl-[1,3,2]dioxaborolane (3.33 g), tripotassium phosphate (8.42 g), DMF (78 mL), and water (24 mL) were mixed. The reaction mixture was degassed by bubbling argon, and then PdCl₂(PPh₃)₂ (1.04 g) was added to the mixture. The resulting mixture was stirred for 2 hrs at 80° C. To the reaction mixture were added water, hexane, and diethyl ether. The mixture was extracted with a mixture solvent of hexane and diethyl ether. The organic layer was washed with brine, and dried with magnesium sulfate. The organic layer was washed with water (twice) and brine in this order. The organic layer was dried over sodium sulfate. The magnesium sulfate was filtered off, and the filtrate was concentrated under reduced pressure, and the resulting residue was purified by silica-gel column chromatography (A mixture of ethyl acetate and hexane was used as an eluent. Eluent: ethyl acetate:hexane=1:100, 1:60, 1:50, 1:40 in this order) to give the title compound (5.8318 g).

### F-644-13

### tert-Butyl (S)-3-[4-cyclopropyl-5-(3-isobutyl-cyclobutyl)-isoxazol-3-yl]-6-hydroxy-hexanoate

To a mixture of tetrabutylammonium fluoride (1 M in THF) (9.8 mL) and acetic acid/water=4/1 (0.98 mL) was added a solution of tert-Butyl ((S)-6-(tert-butyl-diphenyl-silanyloxy)-3-[4-cyclopropyl-5-(3-isobutyl-cyclobutyl)-isoxazol-3-yl]-hexanoate (5.7314 g) in THF (57 mL) at ice temperature. The resulting mixture was stirred overnight at room temperature. The reaction mixture was concentrated under reduced pressure, and to the resulting residue was added water, and the mixture was extracted with ethyl acetate. The organic layer was washed with water, and dried with magnesium sulfate. The magnesium sulfate was filtered off, and the filtrate was concentrated under reduced pressure, and the resulting residue was purified by silica-gel column chromatography (A mixture of ethyl acetate and hexane was used as an eluent. Eluent: ethyl acetate:hexane=1:80, 1:60, 1:40, 1:20, 1:8, 1:3 in this order) to give the title compound (2.94 g).

### F-644-14

### tert-Butyl (S)-3-[4-cyclopropyl-5-(3-isobutyl-cyclobutyl)-isoxazol-3-yl]-6-oxo-hexanoate

tert-Butyl (S)-3-[4-cyclopropyl-5-(3-isobutyl-cyclobutyl)-isoxazol-3-yl]-6-hydroxy-hexanoate (2.85 g) and chloroform (60 mL) were mixed. To the mixture were added Dess-Martin periodinane (4.17 g) and sodium hydrogen carbonate (826 mg) at ice temperature. The reaction mixture was stirred for 2 hrs at room temperature, and to the mixture was added aqueous saturated sodium thiosulfate at ice temperature. The mixture was extracted with chloroform, and the organic layer was concentrated under reduced pressure. The resulting residue was extracted with ethyl acetate. The resulting organic layer was washed with aqueous saturated sodium thiosulfate and aqueous saturated sodium hydrogen carbonate in this order. The organic layer was dried over magnesium sulfate. The magnesium sulfate was filtered off, and the filtrate was concentrated under reduced pressure to give the title compound (2.4369 g) as a crude product.

### F-644-15

### 1-tert-Butyl (S)-3-[4-cyclopropyl-5-(3-isobutyl-cyclobutyl)-isoxazol-3-yl]-adipate

tert-Butyl (S)-3-[4-cyclopropyl-5-(3-isobutyl-cyclobutyl)-isoxazol-3-yl]-6-oxo-hexanoate (2.3715 g), THF (58 mL), and water (8 mL) were mixed. To the mixture were added sodium dihydrogen phosphate (1.06 g), sulfamic acid (800 mg), and sodium chlorite (1.13 g) at ice temperature. The reaction mixture was stirred for 2 hrs at room temperature. To aqueous saturated sodium thiosulfate was added the reaction mixture at ice temperature. The mixture was extracted with ethyl acetate, and the resulting organic layer was washed with aqueous saturated sodium thiosulfate, and dried with magnesium sulfate. The magnesium sulfate was filtered off, and the filtrate was concentrated under reduced pressure, and the resulting residue was purified by silica-gel column chromatography (A mixture of ethyl acetate and hexane was used as an eluent. Fluent: ethyl acetate:hexane=1:9, 1:1 in this order) to give the title compound (2.5488 g).

### F-644-16

### tert-Butyl (S)-5-carbamoyl-3-[4-cyclopropyl-5-(3-isobutyl-cyclobutyl)-isoxazol-3-yl]-valerate

1-tert-Butyl (S)-3-[4-cyclopropyl-5-(3-isobutyl-cyclobutyl)-isoxazol-3-yl]-adipate (1.35 g), ammonium chloride (688 mg), HOBt.H₂O (741 mg), triethylamine (1.8 mL), and DMF (12 mL) were mixed. To the mixture was added WSC.HCl (591 mg). The resulting mixture was stirred overnight at room temperature. To the reaction mixture was added aqueous saturated sodium hydrogen carbonate at ice temperature, and the mixture was extracted with ethyl acetate. The organic layer was washed with aqueous sodium hydrogen carbonate and aqueous saturated sodium chloride in this order. The organic layer was dried over magnesium sulfate. The magnesium sulfate was filtered off, and the filtrate was concentrated under reduced pressure, and the resulting residue was purified by silica-gel column chromatography (A mixture of ethyl acetate and hexane was used as an eluent. Eluent: ethyl acetate:hexane=1:10, 1:4, 1:1, and 2:1 in this order) to give the title compound (905.6 mg).

### F-644-17

### tert-Butyl (S)-5-cyano-3-[4-cyclopropyl-5-(3-isobutyl-cyclobutyl)-isoxazol-3-yl]-valerate

tert-Butyl (S)-5-carbamoyl-3-[4-cyclopropyl-5-(3-isobutyl-cyclobutyl)-isoxazol-3-yl]-valerate (867 mg) and THF (10.4 mL) were mixed. To the mixture was added Burgess reagent (1.49 g). The mixture was stirred for 1.5 hrs at room temperature. To the reaction mixture was added water at ice temperature, and the mixture was extracted with ethyl acetate. The organic layer was washed with aqueous saturated sodium chloride, and dried with magnesium sulfate. The magnesium sulfate was filtered off, and the filtrate was concentrated under reduced pressure, and the resulting residue was purified by silica-gel column chromatography (A mixture of ethyl acetate and hexane was used as an eluent. Eluent: ethyl acetate:hexane=1:20, 1:10, 1:8, 1:6 in this order) to give the title compound (716 mg).

### F-644-18

### (S)-5-cyano-3-[4-cyclopropyl-5-(3-isobutyl-cyclobutyl)-isoxazol-3-yl]-valeric acid

tert-Butyl (S)-5-cyano-3-[4-cyclopropyl-5-(3-isobutyl-cyclobutyl)-isoxazol-3-yl]-valerate (371 mg) and chloroform (9.3 mL) were mixed. To the mixture was added trifluoroacetic acid (1.86 mL) at room temperature. The mixture was stirred for 3 hrs, and concentrated under reduced pressure. The resulting residue was azeotroped with toluene, and purified by silica gel thin-layer chromatography (A mixture of methanol and chloroform was used as an eluent. Eluent: methanol:chloroform=1:9) to give the title compound (124.8 mg).

### F-644-19

### (S)-5-cyano-3-[4-cyclopropyl-5-(3-isobutyl-cyclobutyl)-isoxazol-3-yl]-valeric acid (2,4-dimethyl-phenyl)-amide

(S)-5-cyano-3-[4-cyclopropyl-5-(3-isobutyl-cyclobutyl)-isoxazol-3-yl]-valeric acid (103 mg), WSC.HCl (69 mg), HOBt.H₂O (55 mg), and DMF (1.5 mL) were mixed. To the mixture was added 2,4-dimethylaniline (0.041 mL). The resulting mixture was stirred overnight at room temperature. To the reaction mixture was added water, and the mixture was extracted with ethyl acetate. The organic layer was washed with aqueous saturated sodium chloride and water. The organic layer was dried over sodium sulfate. The sodium sulfate was filtered off, and the filtrate was concentrated under reduced pressure, and the resulting valeric acid was purified by silica gel thin-layer chromatography (A mixture of ethyl acetate and hexane was used as an eluent. Eluent: ethyl acetate:hexane=1:2) to give the title compound (91.9 mg).

Further, the title compound (57.7 mg) was prepared in the same way.

### F-644-20

### (S)-3-[4-Cyclopropyl-5-(3-isobutyl-cyclobutyl)-isoxazol-3-yl]-5-(1H-tetrazole-5-yl)-valeric acid (2,4-dimethyl-phenyl)-amide

(S)-5-Cyano-3-[4-cyclopropyl-5-(3-isobutyl-cyclobutyl)-isoxazol-3-yl]-valeric acid (2,4-dimethyl-phenyl)-amide (140 mg) and xylene (1.7 mL) were mixed. To the mixture was added trimethyltin azide (234 mg), and the mixture was stirred for 2.5 hrs at 125° C. To the reaction mixture was added water at room temperature, and the mixture was extracted with ethyl acetate. The organic layer was washed with water. The organic layer was dried over sodium sulfate. The sodium sulfate was filtered off, and the filtrate was concentrated under reduced pressure, the resulting residue was purified by silica gel thin-layer chromatography (A mixture of methanol and chloroform was used as an eluent. Eluent: methanol:chloroform=3:25) to give the title compound (67 mg).

### Example F-682

### (S)-5-(2-chloro-4-methyl-phenylcarbamoyl)-4-{5-cyclopropyl-4-[3-(2,2-dimethyl-propyl)-cyclobutyl]-[1,2,3]triazol-1-yl}pentanoic acid

### F-682-1

### 4,4-dimethyl-2-pentenoic acid

Ethyl diethylphosphonoacetate (516 mL) was added dropwise to a solution of 20 w/w % sodium ethoxide in ethanol (1.02 L) at ice temperature. The mixture was stirred for 1.5 hrs at ice temperature. A solution of 2,2-dimethylpropionaldehyde (260 mL) in tetrahydrofuran (510 mL) was added dropwise to the mixture at ice temperature. The mixture was stirred for 3.5 hrs at room temperature. To the mixture was added aqueous 4 N sodium hydroxide (885 mL) at ice temperature. The resulting mixture was stirred overnight at room temperature. To the reaction mixture was added 6 N hydrochloric acid (802 mL) at ice temperature. To the mixture was added ethyl acetate (1 L). The organic layer was separated, and washed with water (1 L×5), brine (500 mL) in this order. The organic layer was dried over magnesium sulfate. The magnesium sulfate was filtered off, and the filtrate was concentrated under reduced pressure to give the title compound (289 g) as a crude product.

In the same way, 2,2-dimethylpropionaldehyde (50 mL) was treated to give the title compound (54 g) as a crude product.

### F-682-2

### 4,4-dimethyl valerate

4,4-dimethyl-2-pentenoic acid (343 g), methanol/tetrahydrofuran=3/1 (150 mL), and ethanol (1240 mL) were mixed. To the mixture was added 10 w/w % palladium on activated carbon (31 g). The mixture was stirred for 10.5 hrs at room temperature under hydrogen atmosphere (1 atm). The 10 w/w % palladium on activated carbon was filtered off, and the filtrate was concentrated under reduced pressure to give the title compound (354 g) as a crude product.

### F-682-3

### 4,4-dimethyl-1-piperidine-1-ylpentan-1-one

4,4-dimethyl valerate (348 g), piperidine (291 mL), and DMF (1.7 L) were mixed. To the mixture were added HOBt.H₂O (450 g) and WSC.HCl (563 g) at ice temperature. The resulting mixture was stirred overnight at room temperature. To the reaction mixture was added water (1.7 L) at ice temperature. The mixture was extracted with toluene (500 mL, 400 mL×2). The organic layer was washed with aqueous 10 w/v % sodium carbonate (1 L) and water (1 L) in this order, and concentrated under reduced pressure to give the title compound (508 g) as a crude product.

### F-682-4

### 1-(4,4-dimethyl-1-pentenyl)piperidine

4,4-dimethyl-1-piperidine-1-ylpentan-1-one (508 g) and toluene (1220 mL) were mixed. To the mixture was added (Ph₃P)IrCl(CO) (802 mg). 1,1,3,3-tetramethyldisiloxane (795 mL) was added dropwise to the mixture under water-cooling. The mixture was stirred for 3 hrs at room temperature. The reaction mixture was concentrated under reduced pressure to give the title compound (1171 g) as a crude product.

### F-682-5

### Ethyl 3-(2,2-dimethylpropyl)-2-piperidine-1-ylcyclobutanecarboxylate

1-(4,4-Dimethyl-1-pentenyl)piperidine (1146 g) and acetonitrile (910 mL) were mixed. To the mixture were added ethyl acrylate (549 mL) and hydroquinone (553 mg). The resulting mixture was stirred at 90° C. overnight. The reaction mixture was concentrated under reduced pressure to give the title compound (1470 g) as a crude product.

### F-682-6

### 3-(2,2-Dimethylpropyl)-1-cyclobutenecarboxylic acid

Ethyl 3-(2,2-dimethylpropyl)-2-piperidine-1-ylcyclobutanecarboxylate (1470 g) and methyl p-toluenesulfonate (417 mL) were mixed. The mixture was stirred for 2 hrs at 105° C. To the reaction mixture was added water (2100 mL). The mixture was washed with a mixture of tert-butyl methyl ether:hexane=1:1 (800 mL) and hexane (600 mL) in this order. To the aqueous layer was added potassium hydroxide (663 g) at ice temperature. The mixture was stirred for 2 hrs at 100° C. The reaction mixture was washed with a mixture of tert-butyl methyl ether:hexane=1:1 (600 mL×2). To the aqueous layer were added concentrated hydrochloric acid (500 mL) and 6 N hydrochloric acid (606 mL) at ice temperature. The mixture was extracted with ethyl acetate (600 mL×2). The organic layer was washed with water (1 L×2) and brine (500 mL) in this order. The organic layer was dried over magnesium sulfate. The magnesium sulfate was filtered off, and the filtrate was concentrated under reduced pressure to give the title compound (278 g) as a crude product.

### F-682-7

### 3-(2,2-dimethylpropyl)cyclobutanecarboxylic acid

3-(2,2-Dimethylpropyl)-1-cyclobutenecarboxylic acid (163 g) and tetrahydrofuran (1300 mL) were mixed. To the mixture was added 5 w/w % rhodium on activated carbon (8.2 g). The mixture was stirred for 35 hrs at room temperature under hydrogen atmosphere (1 atm). The 5 w/w % rhodium on activated carbon was filtered off, and the filtrate was concentrated under reduced pressure to give the title compound (175.56 g) as a crude product.

### F-682-8

### 3-(2,2-dimethylpropyl)cyclobutanecarboxylic acid methoxymethylamide

3-(2,2-dimethylpropyl)cyclobutanecarboxylic acid (75.2 g) and DMF (600 mL) were mixed. To the mixture were added N,O-dimethylhydroxylamine hydrochloride salt (51.7 g), triethylamine (92.4 mL), HOBt.H₂O (81.2 g), and WSC.HCl (101.6 g). The resulting mixture was stirred overnight at room temperature. To the reaction mixture was added water. The mixture was extracted with a mixture of ethyl acetate:hexane=1:1. The organic layer was washed with 1 N hydrochloric acid, water, aqueous 10 w/v % sodium carbonate, and water in this order. The organic layer was dried over sodium sulfate. The sodium sulfate was filtered off, and the filtrate was concentrated under reduced pressure to give the title compound (95.2 g) as a crude product.

### F-682-9

### 3-(2,2-dimethylpropyl)cyclobutanecarbaldehyde

Diisobutylaluminium hydride (1.0 M in toluene) (486 mL) was added dropwise to a solution of 3-(2,2-dimethylpropyl)cyclobutanecarboxylic acid methoxymethylamide (95.2 g) in toluene (330 mL) at −78° C. The mixture was stirred for 3 hrs at −78° C., and then 1.5 M sulfuric acid (648 mL) was added dropwise to the mixture at ice temperature. The aqueous layer was separated, and extracted with toluene. The combined organic layer was washed with 1 M sulfuric acid, water, and brine in this order. The organic layer was dried over sodium sulfate. The sodium sulfate was filtered off. The filtrate comprising the title compound was directly used in the next step.

### F-682-10

### 1-(2,2-dibromovinyl)-3-(2,2-dimethylpropyl)cyclobutane

A solution of triphenylphosphine (325 g) in methylene chloride (350 mL) was added dropwise to a solution of carbon tetrabromide (205 g) in methylene chloride (600 mL) at ice temperature. The mixture was stirred for 45 min at ice temperature. A solution of 3-(2,2-dimethylpropyl)cyclobutanecarbaldehyde in toluene was added dropwise to the mixture at ice temperature. The mixture was stirred for 1 hr at ice temperature. Aqueous 10 w/v % sodium carbonate (660 mL) was added dropwise to the mixture at ice temperature. The resultant precipitate was filtered off. The aqueous layer was separated, and extracted with chloroform. The organic layer was washed with water and brine in this order. The organic layer was dried over sodium sulfate. Silica gel was added thereto and the mixture was stirred at room temperature. The sodium sulfate and silica gel were filtered off, and the filtrate was concentrated under reduced pressure. The resulting residue was purified by silica-gel column chromatography (Hexane was used as an eluent.) to give the title compound (120.33 g).

### F-682-11

### 1-(2,2-dimethyl-propyl)-3-ethynyl-cyclobutane

1-(2,2-dibromovinyl)-3-(2,2-dimethylpropyl)cyclobutane (473.4 g), THF (470 mL), and hexane (940 mL) were mixed. n-Butyllithium (1.65 M in hexane) (1832 mL) was added dropwise to the mixture at −78° C. The reaction mixture was stirred for 1 hr at −78° C., and then a solution of acetic acid (34.6 mL) in THF (19 mL) was added dropwise to the reaction mixture. The cooling bath was removed, and to the reaction mixture was added aqueous 25 w/v % ammonium chloride. The organic layer was separated, and washed with water and aqueous 10 w/v % sodium chloride, and dried with magnesium sulfate. The magnesium sulfate was filtered off, and the filtrate was concentrated under reduced pressure. The resulting residue was purified by silica-gel column chromatography (Hexane was used as an eluent.) to give the title compound (217 g).

### F-682-12

### 4-tert-butyl (S)-2-(3-benzyloxy-propyl)-succinate

In a manner similar to E-58-6, the title compound (40.5 g, containing toluene) was prepared as a crude product from 4-tert-butyl (S)-2-(3-benzyloxy-propyl)-succinate (R)-1-phenyl-ethylamine salt (50 g).

### F-682-13

### tert-Butyl (S)-3-amino-6-benzyloxy-hexanoate

4-tert-butyl (S)-2-(3-benzyloxy-propyl)-succinate (3.00 g) and toluene (60 mL) were mixed. To the mixture were added triethylamine (1.56 mL) and diphenylphosphoryl azide (2.11 mL) at room temperature. The mixture was stirred for 3 hrs at 120° C. To the mixture was added 1 M trimethylsilyloxy sodium (18.6 mL) at ice temperature. The mixture was stirred for 20 minutes at room temperature. The reaction mixture was added to aqueous 5 w/v % citric acid (100 mL) at ice temperature. The organic layer was concentrated under reduced pressure. The aqueous layer was washed with diethyl ether (20 mL×2), and aqueous 4N sodium hydroxide (60 mL) was added thereto, and the mixture was extracted with diethyl ether, and the organic layer was dried with magnesium sulfate. The magnesium sulfate was filtered off, and the filtrate was concentrated under reduced pressure. The resulting residue was purified by silica-gel column chromatography (A mixture of methanol and chloroform was used as an eluent. Eluent: methanol:chloroform=1:50 and 1:15 in this order) to give the title compound (2.30 g).

### F-682-14

### Imidazole-1-sulfonyl azide hydrochloride

Sodium azide (13.0 g) and acetonitrile (200 mL) were mixed. Sulfuryl chloride (16.1 mL) was added dropwise to the mixture at ice temperature. The mixture was stirred at room temperature overnight. To the mixture was added imidazole (25.9 g) at ice temperature. The mixture was stirred for 4 hrs at room temperature. To the mixture was added ethyl acetate. The mixture was washed with water (400 mL×2) and aqueous saturated sodium bicarbonate (400 mL×2) in this order. The organic layer was dried over magnesium sulfate. The magnesium sulfate was filtered off, and to the filtrate was added a solution of hydrochloric acid-ethyl acetate at ice temperature. The resultant precipitate was collected by filtration to give the title compound (27.7 g).

### F-682-15

### tert-Butyl (S)-3-azide-6-benzyloxy-hexanoate

tert-Butyl (S)-3-amino-6-benzyloxy-hexanoate (1.17 g), copper(II) sulfate pentahydrate (10 mg), and methanol (18 mL) were mixed. To the mixture were added potassium carbonate (1.11 g) and imidazole-1-sulfonyl azide hydrochloride which was obtained in F-682-14 (1.01 g) at ice temperature. The mixture was stirred at room temperature overnight. To the mixture were added 1 N hydrochloric acid (16 mL), water and brine at ice temperature. The mixture was extracted with tetrahydrofuran. The organic layer was washed with aqueous ammonia and brine in this order. The organic layer was dried over magnesium sulfate. The magnesium sulfate was filtered off, and the filtrate comprising the title compound was directly used in the next step.

### F-682-16

### tert-Butyl (S)-6-benzyloxy-3-{4-[3-(2,2-dimethyl-propyl)-cyclobutyl]-5-iodo-[1,2,3]triazol-1-yl}-hexanoate

N-Bromosuccinimide (925 mg), copper(I) iodide (914 mg), N,N-diisopropylethylamine (0.766 mL) and tetrahydrofuran (20 mL) were mixed. 1-(2,2-Dimethyl-propyl)-3-ethynyl-cyclobutane which was obtained in F-682-11 (601 mg) and tert-butyl (S)-3-azide-6-benzyloxy-hexanoate which was obtained in F-682-15 as a tetrahydrofuran solution were added dropwise to the mixture over 30 minutes at room temperature. The mixture was stirred for 5 hrs at room temperature, and the reaction mixture was filtered. The filtrate was concentrated under reduced pressure. To the resulting residue was added aqueous 10 w/w % ammonia at ice temperature, and the mixture was extracted with ethyl acetate. The organic layer was washed with water and brine in this order. The organic layer was dried over magnesium sulfate. The magnesium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:12, and 1:10 in this order) to give the title compound (648 mg).

### F-682-17

### tert-Butyl (S)-6-benzyloxy-3-{5-cyclopropyl-4-[3-(2,2-dimethyl-propyl)-cyclobutyl]-[1,2,3]triazol-1-yl}-hexanoate

tert-Butyl (S)-6-benzyloxy-3-{4-[3-(2,2-dimethyl-propyl)-cyclobutyl]-5-iodo-[1,2,3]triazol-1-yl}-hexanoate (445 mg), 2-cyclopropyl-4,4,5,5-tetramethyl-[1,3,2]dioxaborolane (0.20 mL), cesium fluoride (504 mg) and N,N-dimethylacetamide (3.5 mL) were mixed. The mixture was degassed by bubbling argon. To the mixture was added PdCl₂(PPh₃)₂ (77 mg) at room temperature. The mixture was stirred for 5 min, and then stirred for 8 hrs at 80° C. To the reaction mixture was added ethyl acetate at ice temperature, and the mixture was filtered. The filtrate was washed with water and brine in this order, and dried with magnesium sulfate. The magnesium sulfate was filtered off, and the filtrate was concentrated under reduced pressure. The resulting residue was purified by silica-gel column chromatography (A mixture of ethyl acetate and hexane was used as an eluent. Eluent: ethyl acetate hexane=1:15) to give the title compound (110 mg).

### F-682-18

### tert-Butyl (S)-3-{5-cyclopropyl-4-[3-(2,2-dimethyl-propyl)-cyclobutyl]-[1,2,3]triazol-1-yl}-6-hydroxy-hexanoate

tert-Butyl (S)-6-benzyloxy-3-{5-cyclopropyl-4-[3-(2,2-dimethyl-propyl)-cyclobutyl]-[1,2,3]triazol-1-yl}-hexanoate (110 mg) and tetrahydrofuran (1 mL) were mixed. To the mixture was added 10 w/w % palladium on activated carbon (55 mg). The mixture was stirred for 2 hrs at room temperature under hydrogen atmosphere (4 atm). The catalyst was freshened up, and then the mixture was stirred for 2 hrs under hydrogen atmosphere (4 atm). The catalyst was again freshened up, and then the mixture was stirred for 2 hrs under hydrogen atmosphere (4 atm). The 10 w/w % palladium on activated carbon was filtered off, and the filtrate was concentrated under reduced pressure to give the title compound (102 mg) as a crude product.

### F-682-19

### 1-tert-Butyl (S)-3-{5-cyclopropyl-4-[3-(2,2-dimethyl-propyl)-cyclobutyl]-[1,2,3]triazol-1-yl}hexanedioate

tert-Butyl (S)-3-{5-cyclopropyl-4-[3-(2,2-dimethyl-propyl)-cyclobutyl]-[1,2,3]triazol-1-yl}-6-hydroxy-hexanoate (102 mg), acetonitrile (0.5 mL) and 1 M phosphate buffer (0.3 mL) were mixed. To the mixture were added 2,2,6,6-tetramethyl-1-piperidinyloxy radical (TEMPO) (3.4 mg) and sodium chlorite (48.8 mg) at room temperature. Sodium hypochlorite solution (0.04 mL) was added dropwise to the mixture at ice temperature. The mixture was stirred for 3 hrs at room temperature. To the mixture were added aqueous 20 w/v % sodium sulfite (0.41 mL) and aqueous 1 M potassium hydrogen sulfate (0.22 mL). The mixture was extracted with ethyl acetate, and the organic layer was washed with brine, and dried with magnesium sulfate. The magnesium sulfate was filtered off, and the filtrate was concentrated under reduced pressure to give the title compound (102 mg) as a crude product.

### F-682-20

### 1-tert-Butyl 6-methyl (S)-3-{5-cyclopropyl-4-[3-(2,2-dimethyl-propyl)-cyclobutyl]-[1,2,3]triazol-1-yl}hexanedioate

1-tert-butyl (S)-3-{5-cyclopropyl-4-[3-(2,2-dimethyl-propyl)-cyclobutyl]-[1,2,3]triazol-1-yl}hexanedioate (102 mg) and DMF (0.8 mL) were mixed. To the mixture were added methyl iodide (0.027 mL) and potassium carbonate (36 mg) at ice temperature. The mixture was stirred for 4 hrs at room temperature, and then water was added at ice temperature. The mixture was extracted with ethyl acetate, and the organic layer was washed with water and brine in this order. The organic layer was dried over magnesium sulfate. The magnesium sulfate was filtered off, and the filtrate was concentrated under reduced pressure to give the title compound (99 mg) as a crude product.

### F-682-21

### 6-Methyl (S)-3-{5-cyclopropyl-4-[3-(2,2-dimethyl-propyl)-cyclobutyl]-[1,2,3]triazol-1-yl}hexanedioate

1-tert-butyl 6-methyl (S)-3-{5-cyclopropyl-4-[3-(2,2-dimethyl-propyl)-cyclobutyl]-[1,2,3]triazol-1-yl}hexanedioate (99 mg) and chloroform (0.5 mL) were mixed. To the mixture was added trifluoroacetic acid (0.2 mL) at ice temperature. The mixture was stirred at room temperature overnight. The reaction mixture was concentrated under reduced pressure, and then the residue was azeotroped with toluene (twice). To the residue was added ethyl acetate, and the organic layer was washed with brine, and dried with magnesium sulfate. The magnesium sulfate was filtered off, and the filtrate was concentrated under reduced pressure to give the title compound (92.6 mg) as a crude product.

### F-682-22

### Methyl (S)-5-(2-chloro-4-methyl-phenylcarbamoyl)-4-{5-cyclopropyl-4-[3-(2,2-dimethyl-propyl)-cyclobutyl]-[1,2,3]triazol-1-yl}pentanoate

6-Methyl (S)-3-{5-cyclopropyl-4-[3-(2,2-dimethyl-propyl)-cyclobutyl]-[1,2,3]triazol-1-yl}hexanedioate (92.6 mg) and dimethylacetamide (0.6 mL) were mixed. To the mixture was added thionyl chloride (0.022 mL) at ice temperature. To the reaction mixture was added 2-chloro-4-methyl-phenylamine (0.029 mL) at ice temperature. The cooling bath was removed, and the reaction mixture was stirred for 1 hr. To the reaction mixture were added ethyl acetate, hexane, and water. The organic layer was separated, and washed with aqueous saturated potassium hydrogen sulfate, aqueous saturated sodium bicarbonate, and brine in this order. The organic layer was dried over magnesium sulfate. The magnesium sulfate was filtered off, and the filtrate was concentrated under reduced pressure to give the title compound (119 mg) as a crude product.

### F-682-23

### (S)-5-(2-chloro-4-methyl-phenylcarbamoyl)-4-{5-cyclopropyl-4-[3-(2,2-dimethyl-propyl)-cyclobutyl]-[1,2,3]triazol-1-yl}pentanoic acid

Methyl (S)-5-(2-chloro-4-methyl-phenylcarbamoyl)-4-{5-cyclopropyl-4-[3-(2,2-dimethyl-propyl)-cyclobutyl]-[1,2,3]triazol-1-yl}pentanoate (119 mg) and methanol (0.88 mL) were mixed. To the mixture was added aqueous 2 M sodium hydroxide (0.216 mL) at room temperature. The mixture was stirred for 3 hrs at 55° C., and then to the mixture was added aqueous 2 M hydrochloric acid (0.216 mL) at room temperature. The mixture was extracted with ethyl acetate, and the organic layer was washed with water and brine in this order. The organic layer was dried over magnesium sulfate. The magnesium sulfate was filtered off, and the filtrate was concentrated under reduced pressure. The resulting residue was purified by silica-gel column chromatography (A mixture of methanol and chloroform was used as an eluent. Eluent: methanol:chloroform=1:70) to give the title compound (41.5 mg).

### Example F-684

### (S)-4-(2-chloro-4-methyl-phenylcarbamoyl)-5-{4-cyclopropyl-5-[3-(2,2-dimethyl-propyl)-cyclobutyl]-isoxazol-3-yl}-pentanoic acid

### F-684-1

### N-Methoxy-N-methyl-succinamic acid tert-butyl ester

Mono-tert-butyl succinate (17.4 g) and N,O-dimethylhydroxylamine hydrochloride salt (11.7 g), HOBt.H₂O (3.1 g), and acetonitrile (35 mL) were mixed. To the mixture was added diisopropylethylamine (23.8 mL) at ice temperature. To the reaction mixture was added WSC.HCl (23.0 g) in three portions at ice temperature. The mixture was stirred for 2 hrs at room temperature. Water was added to the reaction mixture. The mixture was extracted with toluene. The organic layer was washed with aqueous 10 w/v % potassium hydrogen sulfate, brine, aqueous saturated sodium bicarbonate, and brine in this order. The organic layer was dried over magnesium sulfate. The magnesium sulfate was filtered off, and The organic layer was concentrated under reduced pressure to give the title compound (25.2 g, containing toluene (13.4 w/w %), by NMR) as a crude product.

### F-684-2

### tert-Butyl 4-oxo-butanoate

N-Methoxy-N-methyl-succinamic acid tert-butyl ester (10.0 g, equivalent to 39.9 mmol) and THF (100 mL) were mixed. Diisobutylaluminium hydride (1.0 M in toluene) (52 mL) was added dropwise to the mixture at −78° C. The mixture was stirred for 1 hr at −78° C. Saturated ammonium chloride was added dropwise to the reaction mixture at ice temperature. To the mixture was added diethyl ether and water. The organic layer was separated, and washed with aqueous saturated ammonium chloride and brine in this order. The organic layer was dried over magnesium sulfate. The magnesium sulfate was filtered off, and the organic layer was concentrated under reduced pressure until being reduced to approximately 60 mL in volume. The resulting the solution was directly used in the next step.

### F-684-3

### tert-4-hydroxyimino-butanoate

Hydroxylammonium chloride (3.32 g), ethanol (40 mL), and water (12 mL) were mixed. Aqueous 4 M sodium hydroxide (12 mL) was added dropwise to the mixture at ice temperature. A solution of tert-butyl 4-oxo-butanoate in toluene-THF (equivalent to 39.9 mmol), ethanol (10 mL), and THF (12 mL) were added dropwise to the reaction mixture at ice temperature. The mixture was stirred for 1 hr at ice temperature, and then toluene was added to the mixture. The organic layer was separated, and washed with water and brine in this order. The organic layer was dried over magnesium sulfate. The magnesium sulfate was filtered off, and the filtrate was concentrated under reduced pressure to give the title compound (7.60 g, containing toluene (25.9 w/w %), by NMR) as a crude product.

### F-684-4

### tert-Butyl 4-(chloro-hydroxyimino)-butanoate

tert-4-hydroxyimino-butanoate (7.60 g) and DMF (30 mL) were mixed. To the mixture was added N-chlorosuccinimide (4.56 g) under water-cooling. The mixture was stirred for 30 minutes at room temperature. To the reaction mixture were added toluene and water. The organic layer was separated, and washed with water and brine in this order, and the solution was directly used in the next step.

### F-684-5

### tert-Butyl 3-{5-[3-(2,2-dimethyl-propyl)-cyclobutyl]-isoxazol-3-yl}-propionate

1-(2,2-Dimethylpropyl)-3-ethynylcyclobutane which was obtained in F-682-11 (6.60 g), toluene (30 mL), water (18 mL), and potassium carbonate (4.71 g) were mixed. The mixture was heated to 120° C. A solution of tert-butyl 4-(chloro-hydroxyimino)-butanoate which was obtained in F-684-4 in toluene (equivalent to 32.5 mmol) was added dropwise to the reaction mixture, and the resulting mixture was stirred for 1 hr. Water was added to the reaction mixture. The organic layer was separated, and washed with water and brine in this order. The organic layer was dried over magnesium sulfate. The magnesium sulfate was filtered off, and the filtrate was concentrated under reduced pressure to give the title compound (11.4 g, containing toluene (8.2 w/w %), by NMR) as a crude product.

### F-684-6

### tert-Butyl 3-{4-bromo-5-[3-(2,2-dimethyl-propyl)-cyclobutyl]-isoxazol-3-yl}-propionate

tert-Butyl 3-{5-[3-(2,2-dimethyl-propyl)-cyclobutyl]-isoxazol-3-yl}-propionate (11.4 g) and DMF (60 mL) were mixed. To the mixture was added N-bromosuccinimide (6.43 g) at room temperature. The reaction mixture was stirred for 1 hr at 60° C., and cooled to room temperature. To the mixture was added N-bromosuccinimide (6.00 g). The reaction mixture was again heated for 1 hr at 60° C., and then water was added to the reaction mixture. The mixture was extracted with hexane. The organic layer was washed with aqueous 10 w/v % sodium sulfite and brine in this order. The organic layer was dried over magnesium sulfate. The magnesium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:50) to give the title compound (10.58 g).

### F-684-7

### tert-Butyl 3-{4-cyclopropyl-5-[3-(2,2-dimethyl-propyl)-cyclobutyl]-isoxazol-3-yl}-propionate

tert-Butyl 3-{4-bromo-5-[3-(2,2-dimethyl-propyl)-cyclobutyl]-isoxazol-3-yl}-propionate (10.58 g), 2-cyclopropyl-4,4,5,5-tetramethyl-[1,3,2]dioxaborolane (6.66 g), cesium fluoride (18.1 g), PdCl₂ (dppf)₂.CH₂Cl₂ (1.08 g), and NMP (61 mL) were mixed. The reaction mixture was stirred for 2 hrs at 90° C. under argon atmosphere. To the reaction mixture were added water and hexane. The mixture was filtered. The organic layer was separated, washed with water (twice) and brine in this order, and dried with magnesium sulfate. The magnesium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:20) to give the title compound (9.90 g).

### F-684-8

### 3-{4-cyclopropyl-5-[3-(2,2-dimethyl-propyl)-cyclobutyl]-isoxazol-3-yl}-propionic acid

tert-Butyl 3-{4-cyclopropyl-5-[3-(2,2-dimethyl-propyl)-cyclobutyl]-isoxazol-3-yl}-propionate (9.90 g) and toluene (26 mL) were mixed. To the mixture was added trifluoroacetic acid (13.0 mL) at ice temperature. The mixture was stirred at room temperature overnight. The reaction mixture was concentrated under reduced pressure. The resulting residue azeotroped with toluene (twice), and then dissolved in a mixture solvent of hexane-ethyl acetate. The mixture was washed with water (five times) and brine in this order, and dried with magnesium sulfate. The magnesium sulfate was filtered off, and the filtrate was concentrated under reduced pressure to give the title compound (8.09 g, containing toluene (2.0 w/w %) and ethyl acetate (5.5 w/w %), by NMR) as a crude product.

### F-684-9

### (S)-4-benzyl-3-(3-{4-cyclopropyl-5-[3-(2,2-dimethyl-propyl)-cyclobutyl]-isoxazol-3-yl}-propionyl)-oxazolidin-2-one

3-{4-cyclopropyl-5-[3-(2,2-dimethyl-propyl)-cyclobutyl]-isoxazol-3-yl}-propionic acid (4.32 g) and acetonitrile (30 mL) were mixed. To the mixture was added (S)-4-benzyl-2-oxazolidinone (2.44 g). To the mixture were added 4-dimethylaminopyridine (400 mg) and WSC.HCl (2.76 g) at ice temperature. The resulting mixture was stirred overnight at room temperature. Water was added to the reaction mixture. The mixture was extracted with ethyl acetate. The organic layer was washed with water and brine in this order. The organic layer was dried over magnesium sulfate. The magnesium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:2) to give the title compound (4.80 g).

### F-684-10

### (S)-4-benzyl-3-((S)-2-{4-cyclopropyl-5-[3-(2,2-dimethyl-propyl)-cyclobutyl]-isoxazol-3-ylmethyl}-pent-4-enoyl)-oxazolidin-2-one

((S)-4-benzyl-3-(3-{4-cyclopropyl-5-[3-(2,2-dimethyl-propyl)-cyclobutyl]-isoxazol-3-yl}-propionyl)-oxazolidin-2-one (1.50 g) and THF (15 mL) were mixed. Sodium hexamethyldisilazide (1.9 M in THF) (2.0 mL) was added dropwise to the mixture at −78° C. Allyl iodide (0.34 mL) was added dropwise to the reaction mixture at −78° C. The mixture was stirred for 2 hrs. To the reaction mixture were added 2 M hydrochloric acid and water at ice temperature. The mixture was extracted with ethyl acetate. The organic layer was washed with brine, and dried with magnesium sulfate. The magnesium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:4) to give the title compound (1.05 g).

### F-684-11

### (S)-4-Benzyl-3-((S)-2-{4-cyclopropyl-5-[3-(2,2-dimethyl-propyl)-cyclobutyl]-isoxazol-3-ylmethyl}-5-hydroxy-pentanoyl)-oxazolidin-2-one

(S)-4-benzyl-3-((S)-2-{4-cyclopropyl-5-[3-(2,2-dimethyl-propyl)-cyclobutyl]-isoxazol-3-ylmethyl}-pent-4-enoyl)-oxazolidin-2-one (1.05 g) and THF (14 mL) were mixed. 9-Borabicyclo[3.3.1]nonane (0.5 M in THF) (10.0 mL) was added dropwise to the mixture at ice temperature. The reaction mixture was stirred overnight at room temperature. Sodium acetate (409 mg), water (1 mL), and 30 w/w % aqueous hydrogen peroxide (4 mL) were added dropwise to the mixture at ice temperature. The reaction mixture was stirred for 2 hrs at room temperature. To the mixture were added aqueous sodium hydrogen sulfite and aqueous sodium hydrogen sulfate at ice temperature. Ethyl acetate was added to the reaction mixture. The organic layer was separated, washed with brine, and dried with magnesium sulfate. The magnesium sulfate was filtered off, and the filtrate was concentrated under reduced pressure. The resulting residue was purified by silica gel chromatography (A mixture of ethyl acetate and hexane was used as an eluent. Eluent: ethyl acetate:hexane=1:4) to give the title compound (724 mg).

### F-684-12

### (S)-5-((S)-4-benzyl-2-oxo-oxazolidine-3-yl)-4-{4-cyclopropyl-5-[3-(2,2-dimethyl-propyl)-cyclobutyl]-isoxazol-3-ylmethyl}-5-oxo-pentanoic acid

(S)-4-benzyl-3-((S)-2-{4-cyclopropyl-5-[3-(2,2-dimethyl-propyl)-cyclobutyl]-isoxazol-3-ylmethyl}-5-hydroxy-pentanoyl)-oxazolidin-2-one (724 mg), acetonitrile (10 mL), and 1.0 M phosphate buffer (pH=6.8) (1.5 mL) were mixed. To the mixture were added 2,2,6,6-tetramethyl-1-piperidinyloxy radical (TEMPO) (11.0 mg) and sodium chlorite (359 mg) at room temperature. Aqueous sodium hypochlorite (Wako Pure Chemical Industries, 0.22 mL) was added dropwise to the reaction mixture at ice temperature. The mixture was stirred for 3 hrs at room temperature. To the reaction mixture were added aqueous 20 w/v % sodium sulfite and aqueous potassium hydrogen sulfate at ice temperature. The mixture was extracted with ethyl acetate. The organic layer was washed with water and brine in this order. The organic layer was dried over magnesium sulfate. The magnesium sulfate was filtered off, and the filtrate was concentrated under reduced pressure to give the title compound (785 mg) as a crude product.

### F-684-13

### Ethyl (S)-5-((S)-4-benzyl-2-oxo-oxazolidine-3-yl)-4-{4-cyclopropyl-5-[3-(2,2-dimethyl-propyl)-cyclobutyl]-isoxazol-3-ylmethyl}-5-oxo-pentanoate

(S)-5-((S)-4-benzyl-2-oxo-oxazolidine-3-yl)-4-{4-cyclopropyl-5-[3-(2,2-dimethyl-propyl)-cyclobutyl]-isoxazol-3-ylmethyl}-5-oxo-pentanoic acid (785 mg) and DMF (5 mL) were mixed. To the mixture were added ethyl iodide (0.17 mL) and potassium carbonate (384 mg) at room temperature. The mixture was stirred for 3 hrs, and then ethyl acetate was added to the reaction mixture. The organic layer was separated, and washed with water and brine in this order. The organic layer was dried over magnesium sulfate. The magnesium sulfate was filtered off, and the filtrate was concentrated under reduced pressure. The resulting residue was purified by silica gel chromatography (A mixture of ethyl acetate and hexane was used as an eluent. Eluent: ethyl acetate:hexane=1:4) to give the title compound (779 mg).

### F-684-14

### 5-Ethyl (S)-2-{4-cyclopropyl-5-[3-(2,2-dimethyl-propyl)-cyclobutyl]-isoxazol-3-ylmethyl}-pentanedicarboxylate

Lithium hydroxide monohydrate (64 mg), THF (1 mL), and water (4 mL) were mixed. Aqueous 30 w/w % hydrogen peroxide (0.21 mL) was added dropwise to the mixture at ice temperature. The reaction mixture was stirred for 30 minutes at ice temperature. A solution of ethyl (S)-5-((S)-4-benzyl-2-oxo-oxazolidine-3-yl)-4-{4-cyclopropyl-5-[3-(2,2-dimethyl-propyl)-cyclobutyl]-isoxazol-3-ylmethyl}-5-oxo-pentanoate (778 mg) in THF (3 mL) was added dropwise to the reaction mixture at ice temperature. The reaction mixture was stirred for 3 hrs at room temperature. To the mixture were added aqueous 10 w/v % sodium sulfite and 2 M hydrochloric acid at ice temperature. The mixture was extracted with ethyl acetate. The organic layer was washed with brine, and dried with magnesium sulfate. The magnesium sulfate was filtered off, and the filtrate was concentrated under reduced pressure. The resulting residue was purified by silica gel chromatography (A mixture of ethyl acetate and hexane was used as an eluent. Eluent: ethyl acetate:hexane=1:2) to give the title compound (528 mg).

### F-684-15

### Ethyl (S)-4-(2-chloro-4-methyl-phenylcarbamoyl)-5-{4-cyclopropyl-5-[3-(2,2-dimethyl-propyl)-cyclobutyl]-isoxazol-3-yl}-pentanoate

5-Ethyl (S)-2-{4-cyclopropyl-5-[3-(2,2-dimethyl-propyl)-cyclobutyl]-isoxazol-3-ylmethyl}-pentanedicarboxylate (80 mg) and dimethylacetamide (1.0 mL) were mixed. To the mixture was added thionyl chloride (0.019 mL) at ice temperature. To the reaction mixture was added 2-chloro-4-methyl-phenylamine (30 mg) at ice temperature. The cooling bath was removed, and the reaction mixture was stirred for 1 hr. Water was added to the reaction mixture. The mixture was extracted with ethyl acetate. The organic layer was washed with water and brine in this order. The organic layer was dried over magnesium sulfate. The magnesium sulfate was filtered off, and the filtrate was concentrated under reduced pressure to give the title compound (118 mg) as a crude product.

### F-684-16

### (S)-4-(2-chloro-4-methyl-phenylcarbamoyl)-5-{4-cyclopropyl-5-[3-(2,2-dimethyl-propyl)-cyclobutyl]-isoxazol-3-yl}-pentanoic acid

Ethyl (S)-4-(2-chloro-4-methyl-phenylcarbamoyl)-5-{4-cyclopropyl-5-[3-(2,2-dimethyl-propyl)-cyclobutyl]-isoxazol-3-yl}-pentanoate (118 mg) and methanol (0.5 mL) were mixed. To the mixture was added aqueous 2 M sodium hydroxide (0.2 mL) at room temperature. The mixture was stirred for 30 minutes at 80° C. Then, to the mixture were added aqueous 2 M hydrochloric acid (0.2 mL) and water at room temperature. The mixture was extracted with ethyl acetate. The organic layer was washed with brine, and dried with magnesium sulfate. The magnesium sulfate was filtered off, and the filtrate was concentrated under reduced pressure. The resulting residue was purified by preparative chromatography (A mixture of methanol and chloroform was used as an eluent. Eluent: methanol:chloroform=1:20) to give the title compound (82.0 mg).

### Example F-688

### 4-((3R,4R)-3-(2-Chloro-4-methyl-phenylcarbamoyl)-4-{4-cyclopropyl-5-[3-(2,2-dimethyl-propyl)-cyclobutyl]-isoxazol-3-yl}pyrrolidin-1-yl)-4-oxo-butanoic acid

### F-688-1

### Ethyl (E)-4-((R)-4-benzyl-2-oxo-oxazolidine-3-yl)-4-oxo-but-2-enoate

Monoethyl fumarate (10 g) and chloroform (200 mL) were mixed. To the mixture were added (R)-4-benzyl-2-oxazolidinone (13.5 g), 2-chloro-1-methylpyridinium iodide (21.4 g), and triethylamine (23.2 mL). The mixture was stirred for 2 hrs at 55° C., and the reaction mixture was concentrated under reduced pressure, and the resulting residue was diluted with diethyl ether. The mixture was washed with aqueous 1 M hydrochloric acid, aqueous saturated sodium hydrogen carbonate, and brine in this order, and dried over sodium sulfate. The sodium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:3) to give the title compound (16.2 g).

### F-688-2

### Ethyl (3R,4R)-1-benzyl-4-((R-4-benzyl-2-oxo-oxazolidine-3-carbonyl)-pyrrolidine-3-carboxylate

Ethyl (E)-4-((R)-4-benzyl-2-oxo-oxazolidine-3-yl)-4-oxo-but-2-enoate (16.2 g) and chloroform (162 mL) were mixed. To the mixture were added N-(methoxymethyl)-N-(trimethylsilylmethyl)benzylamine (16.4 mL) and trifluoroacetic acid (0.82 mL) at ice temperature. The ice bath was removed, and the reaction mixture was stirred overnight. To the reaction mixture were added aqueous saturated sodium hydrogen carbonate and chloroform at ice temperature. The organic layer was separated, and dried over sodium sulfate. The sodium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:2) to give the title compound (10.4 g).

### F-688-3

### 1-tert-Butyl 3-ethyl (3R,4R)-4-((R-4-benzyl-2-oxo-oxazolidine-3-carbonyl)-pyrrolidine-1,3-dicarboxylate

Ethyl (3R,4R)-1-benzyl-4-HR-4-benzyl-2-oxo-oxazolidine-3-carbonyl)-pyrrolidine-3-carboxylate (10.4 g), and ethanol (100 mL) were mixed. To the mixture were added di-tert-butyl dicarbonate (6.24 g) and 20 w/w % palladium hydroxide (1.5 g). The reaction mixture was stirred overnight under hydrogen atmosphere (medium pressure). The reaction container was charged with nitrogen gas, and then palladium hydroxide was filtered off using Celite with washing with ethyl acetate. The filtrate was concentrated under reduced pressure, and the resulting residue was purified by silica-gel column chromatography (A mixture of ethyl acetate and hexane was used as an eluent. Eluent: ethyl acetate:hexane=2:3) to give the title compound (10.58 g).

### F-688-4

### 1-tert-Butyl 3-ethyl (3R,4R)-pyrrolidine-1,3,4-tricarboxylate

1-tert-Butyl 3-ethyl (3R,4R)-4-((R-4-benzyl-2-oxo-oxazolidine-3-carbonyl)-pyrrolidine-1,3-dicarboxylate (10.58 g), acetonitrile (74 mL), and water (32 mL) were mixed. To the mixture were added triethylamine (13.2 mL) and 4-dimethylaminopyridine (290 mg). The mixture was stirred for 1.5 hrs at 80° C., overnight at room temperature, and then for 3 hrs at 80° C. The reaction mixture was concentrated under reduced pressure, and the resulting residue was diluted with ethyl acetate. The mixture was washed with aqueous 0.5 M hydrochloric acid, water, and brine in this order, and dried over sodium sulfate. The sodium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:1) to give the title compound (5.03 g).

### F-688-5

### 1-tert-Butyl 3-ethyl (3R,4R)-4-(methoxy-methyl-carbamoyl)-pyrrolidine-1,3-dicarboxylate

1-tert-Butyl 3-ethyl (3R,4R)-pyrrolidine-1,3,4-tricarboxylate (5.03 g), and acetonitrile (15 mL) were mixed. To the mixture were added N,O-dimethylhydroxylamine hydrochloride salt (2.05 g) and HOBt.H₂O (804 mg). Diisopropylethylamine (4.17 mL) was slowly added dropwise to the mixture at ice temperature, and then WSC.HCl (4.03 g) was added. The mixture was stirred at room temperature overnight. The reaction mixture was diluted with toluene. To the mixture was added aqueous 1 M hydrochloric acid at ice temperature. The organic layer was separated, and washed with water, aqueous saturated sodium hydrogen carbonate (twice), and brine in this order. The organic layer was dried over sodium sulfate. The sodium sulfate was filtered off, and the filtrate was concentrated under reduced pressure to give the title compound (5.63 g) as a crude product.

### F-688-6

### 1-tert-Butyl (3R,4R)-4-(methoxy-methyl-carbamoyl)-pyrrolidine-1,3-dicarboxylate

1-tert-Butyl 3-ethyl (3R,4R)-4-(methoxy-methyl-carbamoyl)-pyrrolidine-1,3-dicarboxylate (5.63 g) and ethanol (56 mL) were mixed. To the mixture was added aqueous 1 M sodium hydroxide (17.89 mL) at ice temperature. The ice bath was removed, and the mixture was stirred for 3 hrs at room temperature. Then the pH of the mixture was adjusted with aqueous 1 M hydrochloric acid to 3. To the reaction mixture was added ethyl acetate. The organic layer was separated, and washed with water and brine. The organic layer was dried over sodium sulfate. The sodium sulfate was filtered off, and the filtrate was concentrated under reduced pressure to give the title compound (5.047 g) as a crude product.

### F-688-7

### Di-tert-butyl (3R,4R)-4-(methoxy-methyl-carbamoyl)-pyrrolidine-1,3-dicarboxylate

1-tert-Butyl (3R,4R)-4-(methoxy-methyl-carbamoyl)-pyrrolidine-1,3-dicarboxylate (5.047 g) and toluene (41 ml) were mixed. While the mixture was heated to reflux, N,N-dimethylformamide di-tert-butyl acetal (25 was added dropwise to the mixture over 10 min. After the addition, the reaction mixture was cooled to room temperature, and concentrated under reduced pressure. The resulting residue was purified by silica-gel column chromatography (A mixture of ethyl acetate and hexane was used as an eluent. Eluent: ethyl acetate:hexane=1:2) to give the title compound (5.47 g).

### F-688-8

### Di-tert-butyl (3R,4R)-4-formyl-pyrrolidine-1,3-dicarboxylate

Di-tert-butyl (3R,4R)-4-(methoxy-methyl-carbamoyl)-pyrrolidine-1,3-dicarboxylate (5.47 g) and THF (50 mL) were mixed. A solution of lithium aluminium hydride (580 mg) in THF (20 mL) was added dropwise to the mixture under nitrogen atmosphere at −78° C. The resulting mixture was stirred for 30 min at −78° C. Water (10 mL) was added to the mixture at ice temperature, and then ethyl acetate and aqueous 1 M hydrochloric acid were added. The organic layer was separated, and washed with aqueous saturated sodium hydrogen carbonate, water, and brine in this order. The organic layer was dried over sodium sulfate. The sodium sulfate was filtered off, and the organic layer was concentrated under reduced pressure to give the title compound (4.78 g) as a crude product.

### F-688-9

### Di-tert-butyl (3R,4R)-4-(hydroxyimino-methyl)-pyrrolidine-1,3-dicarboxylate

Hydroxylammonium chloride (1.38 g), ethanol (10 mL), and water (5 mL) were mixed. Aqueous 4 M sodium hydroxide (5.00 mL) was added dropwise to the mixture at ice temperature. A solution of di-tert-butyl (3R,4R)-4-formyl-pyrrolidine-1,3-dicarboxylate (4.78 g) in ethanol (10 mL) and THF (5 mL) was added dropwise to the reaction mixture at ice temperature. The ice-bath was removed, and the mixture was stirred at room temperature overnight. To the mixture were added ethyl acetate and water. The organic layer was separated, and washed with water and brine in this order. The organic layer was dried over sodium sulfate. The sodium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:2) to give the title compound (2.86 g).

### F-688-10

### Di-tert-butyl (3R,4R)-4-(chloro-hydroxyimino-methyl)-pyrrolidine-1,3-dicarboxylate

Di-tert-butyl (3R,4R)-4-(hydroxyimino-methyl)-pyrrolidine-1,3-dicarboxylate (2.86 g) and DMF (9 mL) were mixed. A solution of N-chlorosuccinimide (1.34 g) in DMF (6 mL) was added dropwise to the mixture at room temperature. The mixture was stirred for 1 hr at room temperature. To the reaction mixture were added toluene and water at ice temperature. The organic layer was separated, and washed with water and brine in this order, and the solution was directly used in the next step.

### F-688-11

### Di-tert-butyl (3R,4R)-4-{5-[3-(2,2-dimethyl-propyl)-cyclobutyl]-isoxazol-3-yl}-pyrrolidine-1,3-dicarboxylate

A solution of di-tert-butyl (3R,4R)-4-(chloro-hydroxyimino-methyl)-pyrrolidine-1,3-dicarboxylate (equivalent to 9.1 mmol) in toluene, 1-(2,2-dimethylpropyl)-3-ethynylcyclobutane (2.19 g) which was obtained in F-682-11, potassium carbonate (1.38 g), and water (6 mL) were mixed at room temperature. The mixture was heated to 110° C. The reaction mixture was heated to reflux for 6 hrs, and cooled to room temperature. To the reaction mixture was added water and ethyl acetate. The organic layer was separated, and washed with brine. The organic layer was dried over sodium sulfate. The sodium sulfate was filtered off, and the filtrate was concentrated under reduced pressure, and the resulting residue was purified by silica-gel column chromatography (A mixture of ethyl acetate and hexane was used as an eluent. Eluent: ethyl acetate:hexane=1:8) to give the title compound (2.27 g).

### F-688-12

### Di-tert-butyl (3R,4R)-4-{5-[3-(2,2-dimethyl-propyl)-cyclobutyl]-4-iodo-isoxazol-3-yl}-pyrrolidine-1,3-dicarboxylate

Di-tert-butyl (3R,4R)-4-{5-[3-(2,2-dimethyl-propyl)-cyclobutyl]-isoxazol-3-yl}-pyrrolidine-1,3-dicarboxylate (2.05 g), and acetonitrile (20 mL) were mixed. To the mixture were added N-iodosuccinimide (1.200 g) and ammonium cerium(IV) nitrate (1.214 g) at room temperature. The reaction mixture was stirred for 1 hr at 50° C., and then the reaction mixture was cooled to room temperature. Ammonium cerium(IV) nitrate (797 mg) was added. The reaction mixture was again heated for 2 hrs at 50° C. To the reaction mixture were added aqueous 20 w/v % sodium sulfite and ethyl acetate at ice temperature. The organic layer was separated, and washed with water and brine, and dried with magnesium sulfate. The magnesium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:12) to give the title compound (1.048 g).

### F-688-13

### Di-tert-butyl (3R,4R)-4-{4-cyclopropyl-5-[3-(2,2-dimethyl-propyl)-cyclobutyl]-isoxazol-3-yl}-pyrrolidine-1,3-dicarboxylate

Di-tert-butyl (3R,4R)-4-{5-[3-(2,2-dimethyl-propyl)-cyclobutyl]-4-iodo-isoxazol-3-yl}-pyrrolidine-1,3-dicarboxylate (1.0 g), 2-cyclopropyl-4,4,5,5-tetramethyl-[1,3,2]dioxaborolane (571 mg), tripotassium phosphate (1.44 g), PdCl₂(PPh₃)₂ (179 mg), and DMF (10 mL) were mixed. The reaction mixture was degassed by bubbling nitrogen gas. The resulting mixture was stirred overnight at 90° C. To the reaction mixture were added ethyl acetate, hexane, and water. The organic layer was separated, and washed with water and brine in this order. The organic layer was dried over sodium sulfate. The sodium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:15) to give the title compound (750 mg).

### F-688-14

### (3R,4R)-4-{4-cyclopropyl-5-[3-(2,2-dimethyl-propyl)-cyclobutyl]-isoxazol-3-yl}-pyrrolidine-3-carboxylic acid hydrochloride salt

Di-tert-butyl (3R,4R)-4-{4-cyclopropyl-5-[3-(2,2-dimethyl-propyl)-cyclobutyl]-isoxazol-3-yl}-pyrrolidine-1,3-dicarboxylate (750 mg) and chloroform (7.5 mL) were mixed. To the mixture was added trifluoroacetic acid (2.2 mL) at ice temperature. The reaction mixture was stirred at room temperature overnight. The reaction mixture was concentrated under reduced pressure, and the residue was repeatedly azeotroped with toluene. To the residue was added a solution of 4 M hydrochloric acid-ethyl acetate (0.56 mL), and the mixture concentrated under reduced pressure. The residue was repeatedly azeotroped with ethyl acetate. To the resulting residue was added a mixture of ethyl acetate:hexane=1:3. The resulting mixture was stirred for 1 hr at room temperature. The resultant precipitate collected by filtration to give the title compound (368 mg).

### F-688-15

### (3R,4R)-4-{4-Cyclopropyl-5-[3-(2,2-dimethyl-propyl)-cyclobutyl]-isoxazol-3-yl}-1-(3-methoxycarbonyl-propionyl)-pyrrolidine-3-carboxylic acid

(3R,4R)-4-{4-Cyclopropyl-5-[3-(2,2-dimethyl-propyl)-cyclobutyl]-isoxazol-3-yl}-pyrrolidine-3-carboxylic acid hydrochloride salt (80 mg) and chloroform (1 mL) were mixed. To the mixture were added pyridine (0.084 mL) and methyl 4-chloro-4-oxobutyrate (0.077 mL) at ice temperature. The reaction mixture was stirred for 30 minutes at room temperature. Water, aqueous 1 M hydrochloric acid and ethyl acetate were added to the mixture at ice temperature. The organic layer was separated, and washed with water and brine in this order. The organic layer was dried over sodium sulfate. The sodium sulfate was filtered off, and the filtrate was concentrated under reduced pressure to give the title compound (126 mg) as a crude product.

### F-688-16

### Methyl 4-((3R,4R)-3-(2-chloro-4-methyl-phenylcarbamoyl)-4-{4-cyclopropyl-5-[3-(2,2-dimethyl-propyl)-cyclobutyl]-isoxazol-3-yl}pyrrolidine-1-yl)-4-oxo-butanoate

(3R,4R)-4-{4-Cyclopropyl-5-[3-(2,2-dimethyl-propyl)-cyclobutyl]-isoxazol-3-yl}-1-(3-methoxycarbonyl-propionyl)-pyrrolidine-3-carboxylic acid (126 mg) and DMA (1.2 mL) were mixed. To the mixture was added thionyl chloride (0.030 mL) at ice temperature. The reaction mixture was stirred for 30 min at ice temperature. To the reaction mixture was added 2-chloro-4-methylaniline (0.051 mL). The reaction mixture was stirred for 2 hrs at room temperature. To the reaction mixture were added water and ethyl acetate. The organic layer was separated, and washed with aqueous saturated sodium hydrogen carbonate and brine in this order, and was dried over sodium sulfate. The sodium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:1) to give the title compound (86.9 mg).

### F-688-17

### 4-((3R,4R)-3-(2-Chloro-4-methyl-phenylcarbamoyl)-4-{4-cyclopropyl-5-[3-(2,2-dimethyl-propyl)-cyclobutyl]-isoxazol-3-yl}pyrrolidine-1-yl)-4-oxo-butanoic acid

Methyl 4-((3R,4R)-3-(2-chloro-4-methyl-phenylcarbamoyl)-4-{4-cyclopropyl-5-[3-(2,2-dimethyl-propyl)-cyclobutyl]-isoxazol-3-yl}pyrrolidine-1-yl)-4-oxo-butanoate (86 mg), THF (0.5 mL), and methanol (0.5 mL) were mixed. To the mixture was added aqueous 2 M sodium hydroxide (0.148 mL) at room temperature. The reaction mixture was stirred for 2.5 hrs at room temperature. Aqueous 2 M hydrochloric acid was added to make the mixture acidic. To the reaction mixture was added ethyl acetate. The organic layer was separated, and washed with water and brine in this order. The organic layer was dried over sodium sulfate. The sodium sulfate was filtered off, and the filtrate was concentrated under reduced pressure. The resulting residue was purified by silica-gel column chromatography (A mixture of methanol and chloroform was used as an eluent. Eluent: methanol:chloroform=1:10) to give the title compound (51.0 mg).

### Example F-698

### (S)-5-(2-Chloro-4-methyl-phenylcarbamoyl)-4-[7-(3-methyl-butyl)-4,5,6,7-tetrahydro-benz[d]isoxazol-3-yl]-pentanoic acid

### F-698-1

### 3-Ethoxy-6-(3-methyl-butyl)-cyclohex-2-enone

3-Ethoxy-cyclohex-2-enone (11.2 g) and THF (64 mL) were mixed. Lithium diisopropylamide (2.0 M in THF) (52 mL) was added dropwise to the reaction mixture at −78° C. The mixture was stirred for 30 minutes at −78° C. 1-Iodo-3-methyl-butane (22.0 mL) was added dropwise to the reaction mixture at −78° C. The mixture was stirred overnight at room temperature. To the reaction mixture was added water (100 mL) at ice temperature. The aqueous layer was separated, and extracted with diethyl ether (four times). The combined organic layer was washed with water and brine in this order. The organic layer was dried over magnesium sulfate. The magnesium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:10) to give the title compound (3.68 g).

### F-698-2

### 4-(3-Methyl-butyl)-cyclohex-2-enone

3-Ethoxy-6-(3-methyl-butyl)-cyclohex-2-enone (3.65 g) and toluene (20 mL) were mixed. Diisobutylaluminium hydride (1.0 M in toluene) (20.8 mL) was added dropwise to the mixture at ice temperature. The reaction mixture was stirred for 2 hrs at ice temperature. To the reaction mixture were added methanol (10 mL), water (20 mL), and aqueous 10 w/v % potassium hydrogen sulfate (40 mL) in this order at ice temperature. The aqueous layer was separated, and extracted with toluene. The combined organic layer was washed with aqueous saturated sodium bicarbonate, water, and brine in this order. The organic layer was dried over magnesium sulfate. The magnesium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:40) to give the title compound (2.44 g).

### F-698-3

### tert-Butyl (S)-6-benzyloxy-3-[7-(3-methyl-butyl)-4-oxo-3a,4,5,6,7,7a-hexahydro-benz[d]isoxazol-3-yl]-hexanoate

4-(3-methyl-butyl)-cyclohex-2-enone (2.32 g), toluene (18 mL), and aqueous 1.5 M potassium carbonate (15 mL) were mixed. The mixture was heated to 90° C. A solution of tert-butyl (S)-6-benzyloxy-3-(chloro-hydroxyimino-methyl)-hexanoate obtained by a similar reaction to that described in E-58-10 in toluene (equivalent to 16.7 mmol) was added dropwise to the reaction mixture. The mixture was stirred for 1.5 hrs. To the reaction mixture was added toluene at room temperature. The organic layer was separated, and washed with water and brine in this order. The organic layer was dried over magnesium sulfate. The magnesium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:10) to give the title compound (2.93 g).

### F-698-4

### tert-Butyl (S)-6-benzyloxy-3-[7-(3-methyl-butyl)-4-oxo-4,5,6,7-tetrahydro-benz[d]isoxazol-3-yl]-hexanoate

tert-Butyl (S)-6-benzyloxy-3-[7-(3-methyl-butyl)-4-oxo-3a,4,5,6,7,7a-hexahydro-benz[d]isoxazol-3-yl]-hexanoate (2.90 g) and xylene (20 mL) were mixed. To the reaction mixture was added activated carbon for oxidation (Tokyo Chemical Industry Co., Ltd., 1.45 g) at room temperature. The mixture was stirred for 3 hrs at 150° C. The reaction mixture was filtered, and the filtrate was concentrated under reduced pressure to give the title compound (3.44 g) as a crude product.

### F-698-5

### tert-Butyl (S)-6-benzyloxy-3-[7-(3-methyl-butyl)-4-hydroxy-4,5,6,7-tetrahydro-benz[d]isoxazol-3-yl]-hexanoate

tert-Butyl (S)-6-benzyloxy-3-[7-(3-methyl-butyl)-4-oxo-4,5,6,7-tetrahydro-benz[d]isoxazol-3-yl]-hexanoate (952 mg, equivalent to 1.65 mmol) and methanol (6.4 mL) were mixed. To the mixture was added cerium chloride heptahydrate (656 mg) at ice temperature. The reaction mixture was stirred for 5 min at ice temperature. To the reaction mixture was added sodium borohydride (75.1 mg) at ice temperature. The mixture was stirred for 1 hr. To the reaction mixture were added acetone, water, ethyl acetate in this order at ice temperature. The organic layer was separated, and washed with water and brine in this order. The organic layer was dried over magnesium sulfate. The magnesium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:12) to give the title compound (542 mg).

### F-698-6

### tert-Butyl (S)-6-benzyloxy-3-[4-(imidazole-1-carbothioyloxy)-7-(3-methyl-butyl)-4,5,6,7-tetrahydro-benz[d]isoxazol-3-yl]-hexanoate

tert-Butyl (S)-6-benzyloxy-3-[7-(3-methyl-butyl)-4-hydroxy-4,5,6,7-tetrahydro-benz[d]isoxazol-3-yl]-hexanoate (350 mg) and THF (3.5 mL) were mixed. To the mixture were added 1,1′-thiocarbonyldiimidazole (285 mg) and N,N-dimethyl-4-aminopyridine (17.6 mg) at room temperature. The mixture was stirred for 1 hr at 70° C. The reaction mixture was concentrated under reduced pressure, and the resulting residue was purified by silica-gel column chromatography (A mixture of ethyl acetate and hexane was used as an eluent. Eluent: ethyl acetate:hexane=1:4) to give the title compound (208 mg).

### F-698-7

### tert-Butyl (S)-6-benzyloxy-3-[7-(3-methyl-butyl)-4,5,6,7-tetrahydro-benz[d]isoxazol-3-yl]-hexanoate

tert-Butyl (S)-6-benzyloxy-3-[4-(imidazole-1-carbothioyloxy)-7-(3-methyl-butyl)-4,5,6,7-tetrahydro-benz[d]isoxazol-3-yl]-hexanoate (200 mg) and toluene (2 mL) were mixed. To the mixture were added tri-n-butyltin (0.722 mL) and azobis(isobutyronitrile) (11.0 mg) at room temperature. The reaction mixture was stirred for 7 hrs at 90° C., and concentrated under reduced pressure. The resulting residue was purified by silica-gel column chromatography (A mixture of ethyl acetate and hexane was used as an eluent. Eluent: ethyl acetate:hexane=1:10) to give the title compound (108 mg). The title compound (20.0 mg) was obtained in a similar way.

### F-698-8

### (S)-6-benzyloxy-3-[7-(3-methyl-butyl)-4,5,6,7-tetrahydro-benz[d]isoxazol-3-yl]-hexanoic acid

tert-Butyl (S)-6-benzyloxy-3-[7-(3-methyl-butyl)-4,5,6,7-tetrahydro-benz[d]isoxazol-3-yl]-hexanoate (118 mg) and chloroform (0.600 mL) were mixed. To the mixture was added trifluoroacetic acid (0.240 mL) at ice temperature. The reaction mixture was stirred at room temperature overnight. To the mixture was added toluene. The mixture was concentrated under reduced pressure to give the title compound (111 mg) as a crude product.

### F-698-9

### (S)-6-benzyloxy-3-[7-(3-methyl-butyl)-4,5,6,7-tetrahydro-benz[d]isoxazol-3-yl]-hexanoic acid (2-chloro-4-methyl-phenyl)amide

(S)-6-benzyloxy-3-[4′-cyclopropyl-5-(2,2-dimethyl-propyl)-[3,5′]biisoxazolyl-3′-yl]-hexanoic acid (111 mg, equivalent to 0.251 mmol) and dimethylacetamide (0.730 mL) were mixed. To the mixture was added thionyl chloride (0.020 mL) at ice temperature. To the reaction mixture was added 2-chloro-4-methyl-phenylamine (0.034 mL) at ice temperature. The cooling bath was removed, and the reaction mixture was stirred for 1 hr. To the reaction mixture were added a mixture of ethyl acetate:hexane=1:2, and water. The organic layer was separated, and washed with water and brine in this order, and dried over magnesium sulfate. The magnesium sulfate was filtered off, and the filtrate was concentrated under reduced pressure. The resulting residue was purified by silica-gel column chromatography (A mixture of ethyl acetate and hexane was used as an eluent. Eluent: ethyl acetate:hexane=1:8) to give the title compound (94.1 mg).

### F-698-10

### (S)-6-hydroxy-3-[7-(3-methyl-butyl)-4,5,6,7-tetrahydro-benz[d]isoxazol-3-yl]-hexanoic acid (2-chloro-4-methyl-phenyl)amide

(S)-6-benzyloxy-3-[7-(3-methyl-butyl)-4,5,6,7-tetrahydro-benz[d]isoxazol-3-yl]-hexanoic acid (2-chloro-4-methyl-phenyl)amide (94.1 mg) and dichloromethane (1 mL) were mixed. To the mixture was added boron tribromide (1 M in dichloromethane) (0.526 mL) at ice temperature. The reaction mixture was stirred for 15 min at ice temperature, and then stirred for 20 min at room temperature. To the reaction mixture were added aqueous saturated sodium hydrogen carbonate and chloroform at ice temperature. The organic layer was separated, and washed with water and brine in this order. The organic layer was dried over magnesium sulfate. The magnesium sulfate was filtered off, and the filtrate was concentrated under reduced pressure. The resulting residue was purified by preparative chromatography (A mixture of ethyl acetate and hexane was used as an eluent. Eluent: ethyl acetate:hexane=1:1) to give the title compound (55.9 mg).

### F-698-11

### (S)-5-(2-Chloro-4-methyl-phenylcarbamoyl)-4-[7-(3-methyl-butyl)-4,5,6,7-tetrahydro-benz[d]isoxazol-3-yl]-pentanoic acid

(S)-6-Hydroxy-3-[7-(3-methyl-butyl)-4,5,6,7-tetrahydro-benz[d]isoxazol-3-yl]-hexanoic acid (2-chloro-4-methyl-phenyl)amide (55.2 mg), acetonitrile (0.300 mL), and 1.0 M phosphate buffer (pH=6.8) (0.180 mL) were mixed. To the reaction mixture were added 2,2,6,6-tetramethyl-1-piperidinyloxy radical (TEMPO) (1.9 mg), sodium chlorite (27.8 mg), and aqueous sodium hypochlorite (Wako Pure Chemical Industries, 0.024 mL) at ice temperature. The reaction mixture was stirred for 1 hr at room temperature. To the mixture were added aqueous sodium sulfite and aqueous sodium hydrogen sulfate at ice temperature. Ethyl acetate was added to the reaction mixture. The organic layer was separated, and washed with water and brine in this order. The organic layer was dried over magnesium sulfate. The magnesium sulfate was filtered off, and the filtrate was concentrated under reduced pressure. The resulting residue was purified by preparative chromatography (A mixture of acetic acid, methanol, and chloroform was used as an eluent. Eluent: acetic acid:methanol:chloroform=0.05:1:20) to give the title compound (51.5 mg).

### Example F-715

### (S)-5-(2-Chloro-4-methyl-phenylcarbamoyl)-4-{4-cyclopropyl-3-[3-(2,2-dimethyl-propyl)-cyclobutyl]-isoxazol-5-yl}-pentanoic acid

### F-715-1

### 3-(2,2-Dimethyl-propyl)-cyclobutanecarbaldehyde oxime

To a solution of hydroxylammonium chloride (3.2 g) in water (20 mL) were added ethanol (80 mL) and aqueous 4 N sodium hydroxide (11.5 mL) at ice temperature. A solution of 3-(2,2-dimethyl-propyl)-cyclobutanecarbaldehyde which was obtained in F-682-7 in toluene was added dropwise slowly to the mixture. The mixture was stirred for 12 hrs at room temperature. To the mixture was added ethyl acetate (50 mL). The organic layer was separated, and washed with water (50 mL) and brine (50 mL), and dried over sodium sulfate. The desiccant was filtered off, and the filtrate was concentrated under reduced pressure to give the title compound (6.05 g) as a crude product.

### F-715-2

### tert-Butyl (S)-6-benzyloxy-3-{3-[3-(2,2-dimethyl-propyl)-cyclobutyl]-isoxazol-5-yl}-hexanoate

To a solution of 3-(2,2-dimethyl-propyl)-cyclobutanecarbaldehyde oxime (1.64 g) in DMF (5.0 mL) was added a solution of N-chlorosuccinimide (1.56 g) in DMF (3.0 mL) at ice temperature. The mixture was stirred for 30 minutes at room temperature. To the reaction mixture were added toluene (19 mL) and water (19 mL). The organic layer was separated, and washed with brine (19 mL), and put into a dropping funnel. The organic layer was added dropwise slowly to a mixture of tert-butyl (S)-6-benzyloxy-3-ethynyl-hexanoate which was obtained in E-52-1 (2.94 g), toluene (9.0 mL), potassium carbonate (1.5 g), and water (4.0 mL) over 20 minutes at 100° C. To the reaction mixture were added ethyl acetate (9.0 mL) and water (9.0 mL). The organic layer was separated, and washed with water (9.0 mL) and brine (9.0 mL) and dried over sodium sulfate. The desiccant was filtered off, and the filtrate was concentrated under reduced pressure, and the resulting residue was purified by silica-gel column chromatography (A mixture of ethyl acetate and hexane was used as an eluent. Eluent: ethyl acetate:hexane=1:15) to give the title compound (2.12 g).

### F-715-3

### tert-Butyl (S)-3-{3-[3-(2,2-dimethyl-propyl)-cyclobutyl]-isoxazol-5-yl}-6-hydroxy-hexanoate

To a solution of tert-butyl (S)-6-benzyloxy-3-{3-[3-(2,2-dimethyl-propyl)-cyclobutyl]-isoxazol-5-yl}-hexanoate (2.12 g) in tetrahydrofuran (10 mL) was added 10% palladium on carbon (powder) PE-TYPE (220 mg). The mixture was stirred for 12 hrs at room temperature under hydrogen atmosphere (1 atm). The catalyst was filtered off, and the filtrate was concentrated under reduced pressure to give the title compound (1.86 g) as a crude product.

### F-715-4

### 1-tert-Butyl (S)-3-{3-[3-(2,2-dimethyl-propyl)-cyclobutyl]-isoxazol-5-yl}-hexanedioate

tert-Butyl (S)-3-{3-[3-(2,2-dimethyl-propyl)-cyclobutyl]-isoxazol-5-yl}-6-hydroxy-hexanoate (539 mg) was dissolved in a mixture of acetonitrile (2.7 mL) and 1.0 M phosphate buffer (pH=6.8) (1.1 mL). To the mixture were added 2,2,6,6-tetramethyl-1-piperidinyloxy radical (TEMPO) (11 mg) and sodium chlorite (321 mg) at room temperature. Then, aqueous sodium hypochlorite (0.162 mL) was added dropwise to the mixture. The resulting mixture was stirred for 1 hr at room temperature. Aqueous 20 w/v % sodium sulfite (2.0 mL) was added dropwise to the mixture at ice temperature. Then, ethyl acetate (7.0 mL) and water (2.0 mL) were added. The organic layer was separated, and washed with brine (4.0 mL), and dried over magnesium sulfate. The desiccant was filtered off, and the filtrate was concentrated under reduced pressure to give the title compound (560 mg) as a crude product.

### F-715-5

### 1-tert-Butyl 6-methyl (S)-3-{3-[3-(2,2-dimethyl-propyl)-cyclobutyl]-isoxazol-5-yl}-hexanedioate

1-tert-Butyl (S)-3-{3-[3-(2,2-dimethyl-propyl)-cyclobutyl]-isoxazol-5-yl}-hexanedioate (560 mg) was dissolved in DMF (3.0 mL). To the mixture were added methyl iodide (0.179 mL) and potassium carbonate (236 mg) at ice temperature. The mixture was stirred for 12 hrs at room temperature. To the mixture were added ice water (3.0 mL), n-hexane (2.0 mL), and ethyl acetate (2.0 mL). The resulting organic layer was washed with aqueous 5 w/v % potassium hydrogen sulfate (3.0 mL) and brine (3.0 mL), and dried with magnesium sulfate. The desiccant was filtered off, and the filtrate was concentrated under reduced pressure. The resulting residue was purified by silica-gel column chromatography (A mixture of ethyl acetate and hexane was used as an eluent. Eluent: ethyl acetate:hexane=1:4) to give the title compound (516 mg).

### F-715-6

### 1-tert-Butyl 6-methyl (S)-3-{4-bromo-3-[3-(2,2-dimethyl-propyl)-cyclobutyl]-isoxazol-5-yl}-hexanedioate

1-tert-Butyl 6-methyl (S)-3-{3-[3-(2,2-dimethyl-propyl)-cyclobutyl]-isoxazol-5-yl}-hexanedioate (516 mg) was dissolved in a mixture solvent of DMF:isopropyl acetate=1:1 (3.2 mL). To the mixture were added 1,3-dibromo-5,5-dimethylhydantoin (272 mg) and p-toluenesulfonic acid monohydrate (24 mg) at room temperature. The reaction mixture was stirred for 12 hrs at room temperature. Then, to the mixture were added ice water (5.0 mL) and ethyl acetate (5.0 mL). The organic layer was separated, and washed with aqueous saturated sodium thiosulfate (3.0 mL) and brine (3.0 mL), and dried with magnesium sulfate. The desiccant was filtered off, and the filtrate was concentrated under reduced pressure. The resulting residue was purified by silica-gel column chromatography (A mixture of ethyl acetate and hexane was used as an eluent. Eluent: ethyl acetate:hexane=1:4) to give the title compound (266 mg).

### F-715-7

### 1-tert-Butyl 6-methyl (S)-3-{4-cyclopropyl-3-[3-(2,2-dimethyl-propyl)-cyclobutyl]-isoxazol-5-yl}-hexanedioate

1-tert-butyl 6-methyl (S)-3-{4-bromo-3-[3-(2,2-dimethyl-propyl)-cyclobutyl]-isoxazol-5-yl}-hexanedioate (210 mg) was dissolved in a mixture solvent of toluene:water=10:1 (11 mL). To the mixture were added cyclopropylboronic acid (56 mg), tripotassium phosphate (183 mg), bis{di-tert-butyl(4-dimethylaminophenyl)phosphine}dichloropalladium (II) (15 mg) at room temperature. The reaction mixture was stirred for 12 hrs at 100° C. To the mixture were added ice water (5.0 mL) and ethyl acetate (5.0 mL). The organic layer was separated, and washed with brine (3.0 mL), and dried with magnesium sulfate. The desiccant was filtered off, and the filtrate was concentrated under reduced pressure. The resulting residue was purified by silica-gel column chromatography (A mixture of ethyl acetate and hexane was used as an eluent. Eluent: ethyl acetate:hexane=1:9) to give the title compound (134 mg).

### F-715-8

### 6-Methyl (S)-3-{4-cyclopropyl-3-[3-(2,2-dimethyl-propyl)-cyclobutyl]-isoxazol-5-yl}-hexanedioate

1-tert-Butyl 6-methyl (S)-3-{4-cyclopropyl-3-[3-(2,2-dimethyl-propyl)-cyclobutyl]-isoxazol-5-yl}-hexanedioate (134 mg) was dissolved in toluene (0.6 mL).

Trifluoroacetic acid (0.3 mL) was added dropwise to the mixture at ice temperature. The reaction mixture was stirred for 6 hrs at room temperature, and concentrated under reduced pressure. The resulting residue was azeotroped with toluene (twice) to give the title compound (117 mg) as a crude product.

### F-715-9

### Methyl (S)-5-(2-chloro-4-methyl-phenylcarbamoyl)-4-{4-cyclopropyl-3-[3-(2,2-dimethyl-propyl)-cyclobutyl]-isoxazol-5-yl}-pentanoate

6-Methyl (S)-3-{4-cyclopropyl-3-[3-(2,2-dimethyl-propyl)-cyclobutyl]-isoxazol-5-yl}-hexanedioate (39 mg) was dissolved in DMF (1.5 mL). To the mixture were added 2-chloro-4-methylphenylamine (17 mg) diisopropylethylamine (0.053 mL), and HATU (46 mg) at ice temperature. The mixture was stirred for 12 hrs at room temperature. To the mixture were added ice water (3.0 mL), n-hexane (2.0 mL), and ethyl acetate (2.0 mL). The organic layer was separated, and washed with aqueous 5 w/v % potassium hydrogen sulfate (3.0 mL) and brine (3.0 mL). The organic layer was dried over sodium sulfate. The desiccant was filtered off, and the filtrate was concentrated under reduced pressure. The resulting residue was purified by silica-gel column chromatography (A mixture of ethyl acetate and hexane was used as an eluent. Eluent: ethyl acetate:hexane=1:2) to give the title compound (34 mg).

### F-715-10

### (S)-5-(2-chloro-4-methyl-phenylcarbamoyl)-4-{4-cyclopropyl-3-[3-(2,2-dimethyl-propyl)-cyclobutyl]-isoxazol-5-yl}-pentanoic acid

Methyl (S)-5-(2-chloro-4-methyl-phenylcarbamoyl)-4-{4-cyclopropyl-3-[3-(2,2-dimethyl-propyl)-cyclobutyl]-isoxazol-5-yl}-pentanoate (33 mg) was dissolved in methanol (0.1 mL). To the mixture was added aqueous 2N sodium hydroxide (0.064 mL) at room temperature. The reaction mixture was stirred for 2 hrs at 60° C., and then was concentrated under reduced pressure. To the residue was added 1 N hydrochloric acid (0.128 mL) at ice temperature. Then water (3.0 mL) and ethyl acetate (3.0 mL) were added. The organic layer was separated, and washed with aqueous 5 w/v % potassium hydrogen sulfate (3.0 mL) and brine (3.0 mL). The organic layer was dried over sodium sulfate. The desiccant was filtered off, and the filtrate was concentrated under reduced pressure. The resulting residue was purified by silica-gel column chromatography (A mixture of methanol and chloroform was used as an eluent. Eluent: methanol:chloroform=1:10) to give the title compound (31 mg).

### Production Example of Example F-1

### Production Example of Example F-9

### Production Example of Example F-19

### Production Example of Example F-62

### Production Example of Example F-63

### Production Example of Example F-259

### Production Example of Example F-319

### Production Example of Example F-320

### Production Example of Example F-35

### Production Example of Example F-36

### Production Example of Example F-145

### Production Example of Example F-146

### Production Example of Example F-227

### Production Example of Example F-302

### Production Example of Example F-303

### Production Example of Example F-397

### Production Example of Example F-398

### Production Example of Example F-399

### Production Example of Example F-400

### Production Example of Example F-448

### Production Example of Example F-431

### Production Example of Example F-470

### Production Example of Example F-471

### Production Example of Example F-500

### Production Example of Example F-501

### Production Example of Example F-502

### Production Example of Example F-503

### Production Example of Example F-517

### Production Example of Example F-533

### Production Example of Example F-534

### Production Example of Example F-539

### Production Example of Example F-545

### Production Example of Example F-408

### Production Example of Example F-547

### Production Example of Example F-562

### Production Example of Example F-563

### Production Example of Example F-564

### Production Example of Example F-565

### Production Example of Example F-570

### Production Example of Example F-592

### Production Example of Example F-593

### Production Example of Example F-655

### Production Example of Example F-310

### Production Example of Example F-394

### Production Example of Example F-395

### Production Example of Example F-2

### Production Example of Example F-283

### Production Example of Example F-355

### Production Example of Example F-433

### Production Example of Example F-434

### Production Example of Example F-310

### Production Example of Example F-450

### Production Example of Example F-459

### Production Example of Example F-479

### Production Example of Example F-490

### Production Example of Example F-585

### Production Example of Example F-621

### Production Example of Example F-647

### Production Example of Example F-701

### Production Example of Example F-710

### Production Example of Example F-494

### Production Example of Example F-704

### Production Example of Example F-733

### Production Example of Example F-740

### Production Example of Example F-744

### Production Example of Example F-745

### Production Example of Example F-645

### Production Example of Example F-646

### Production Example of Example F-687

### Production Example of Example F-636

### Production Example of Example F-670

### Production Example of Example F-640

### Production Example of Example F-700

### Production Example of Example F-705

### Production Example of Example F-722

### Production Example of Example F-725

### Production Example of Example F-727

### Production Example of Example F-668

### Production Example of Example F-677

### Production Example of Example F-678

### Production Example of Example F-690

TABLE 112

| | | | MS | |
|---|---|---|---|---|
| | | | M + H | M − H |
| Exam- | | | or | or |
| ple | Chemical structure | NMR | M − Na + H | M − Na − H |
| E-01 | | (400 MHz, CDCl3) 0.52-0.64 (m, 1H), 0.69-0.81 (m, 1H), 0.92-1.06 (m, 2H), 1.00 (d, J = 6.40 Hz, 6H), 1.61-1.74 (m, 1H), 2.01-2.32 (m, 3H), 2.32 (s, 3H), 2.36-2.52 (m, 2H), 2.71 (d, J = 7.20 Hz, 2H), 2.82 (dd, J = 14.80, 5.20 Hz, 1H), 3.07 (dd, J = 14.80, 9.20 Hz, 1H), 3.71-3.84 (m, 1H), | 500 | 498 |
| | | 6.41 (s, 1H), 7.02 (d, J = 8.40 | | |
| | | Hz, 1H), 7.14 (s, 1H), 7.71 | | |
| | | (s, 1H), 8.08 (d, J = 8.40 Hz, | | |
| | | 1H) | | |
| E-02 | | (400 MHz, DMSO-d6) 0.48- 0.55 (m, 1H), 0.63-0.71 (m, 1H), 0.90-0.98 (m, 2H), 0.94 (d, J = 7.20 Hz, 6H), 1.70-1.79 (m, 1H), 1.80-1.97 (m, 4H), 1.98-2.11 (m, 1H), 2.26 (s, 3H), 2.75 (d, J = 7.20 Hz, 2H), 2.79 (dd, J = 14.80, 6.00 Hz, 1H), 2.87 (dd, J = 14.80, 8.40 Hz, 1H), 3.48-3.58 (m, 1H), 6.76 (s, 1H), 7.07 (d, J = 8.40, | 500 | 498 |
| | | 1H), 7.27 (s, 1H), 7.41 (d, J = | | |
| | | 8.40 Hz, 1H), 9.77 (s, 1H) | | |
| E-03 | | (400 MHz, CDCl3) 0.33-0.45 (m, 1H), 0.46-0.59 (m, 1H), 0.67-0.86 (m, 2H), 0.92 (d, J = 6.40 Hz, 6H), 1.44-1.58 (m, 1H), 1.83-2.35 (m, 5H), 2.24 (s, 3H), 2.60 (d, J = 6.80 Hz, 2H), 2.68-2.82 (m, 1H), 2.82-2.96 (m, 1H), 3.44-3.75 (m, 1H), 6.26 (s, 1H), 6.56- 6.76 (m, 2H), 7.62-7.78 (m, 1H), 8.87 (brs, 1H) | 484 | 482 |
| E-04 | | (400 MHz, CDCl3) 0.30-0.59 (m, 2H), 0.73-0.99 (m, 2H), 0.93 (d, J = 6.40 Hz, 6H), 1.41-1.58 (m, 1H), 1.83-2.32 (m, 5H), 2.11 (2.73-2.99 (m, 2H), 3.46-3.71 (m, 1H), 6.41 (s, 1H), 6.66-6.99 (m, 2H), 7.60-7.83 (m, 1H) | 500 | 498 |
| E-05 | | (400 MHz, CDCl3) 0.30-0.61 (m, 2H), 0.66-1.00 (m, 2H), 0.92 (d, J = 5.60 Hz, 6H), 1.42-1.67 (m, 4H), 1.80-234 (m, 4H), 2.12 (s, 3H), 2.54- 3.01 (m, 4H), 344-377 (m, 1H), 6.24 (s, 1H), 6.65-7.03 (m, 2H), 7.64-7.87 (m, 1H) | 514 | 512 |

TABLE 113

| | | | MS | |
|---|---|---|---|---|
| Ex- | | | M + H | M − H |
| am- | | | or | or |
| ple | Chemical structure | NMR | M − Na + H | M − Na − H |
| E-06 | | (400 MHz, DMSO-D6) 0.47- 0.55 (m, 1H), 0.62-0.71 (m, 1H), 0.88-0.95 (m, 2H), 0.95- 1.00 (m, 9H), 1.68-1.95 (m, 4H), 2.21-2.30 (m, 3H), 2.75- 2.79 (m, 2H), 2.79-2.92 (m, 1H), 3.39-349 (m, 1H), 3.49- 3.58 (m, 1H), 4.30-4.40 (m, 1H), 6.73 (s, 1H), 7.05-7.10 (m, 1H), 7.27 (br s, 1H), 7.38-7.44 (m, 1H), 9.83 (br | 514 | 512 |
| | | s, 1H). | | |
| E-07 | | (400 MHz, DMSO-d6) 0.42- 0.55 (m, 1H), 0.60-0.71 (m, 1H), 0.83-0.96 (m, 2H), 0.90 (d, J = 6.40 Hz, 6H), 1.64- 1.73 (m, 1H), 1.86-2.03 (m, 3H), 2.17-2.34 (m, 2H), 2.26 (s, 3H), 2.53 (d, J = 6.80 Hz, 2H), 2.78 (dd, J = 15.20, 6.80 Hz, 1H), 2.90 (dd, J = 15.20, 8.00 Hz, 1H), 3.50- 3.63 (m, 1H), 6.46 (s, 1H), | 483 | 481 |
| | | 6.92 (d, J = 8.00, 1H), 7.04 | | |
| | | (d, J = 12.4 Hz, 1H), 7.58 (t, | | |
| | | J = 8.40 Hz, 1H), 9.69 (s, 1H) | | |
| E-08 | | (400 MHz, DMSO-d6) 0.42- 0.52 (m, 1H), 0.58-0.67 (m, 1H), 0.80-0.92 (m, 2H), 0.88 (d, J = 6.80 Hz, 6H), 1.63- 1.74 (m, 1H), 1.78-1.96 (m, 5H), 2.24 (s, 3H), 2.51 (d, J = 7.20 Hz, 2H), 2.74 (dd, J = 15.20, 6.40 Hz, 1H), 2.83 (dd, J = 15.20, 8.00 Hz, 1H), 3.40-3.52 (m, 1H), 6.42 (s, 1H), 6.89 (d, J = 8.00, 1H), | 483 | 481 |
| | | 7.01 (d, J = 11.2 Hz, 1H), | | |
| | | 7.58 (t, J = 8.00 Hz, 1H), 9.91 | | |
| | | (s, 1H), 13.06 (brs, 1H) | | |
| E-09 | | (400 MHz, CDCl3) 0.31-0.62 (m, 2H), 0.66-0.99 (m, 2H), 0.91 (d, J = 6.00 Hz, 6H), 1.38-1.68 (m, 4H), 1.83-2.26 (m, 2H), 2.12 (s, 3H), 2.32- 2.53 (m, 2H), 2.62-2.98 (m, 4H), 3.43-3.67 (m, 1H), 6.23 (s, 1H), 6.54-6.78 (m, 2H), 7.60-7.78 (m, 1H), 8.84 (brs, 1H) | 498 | 496 |
| E-10 | | (400 MHz, CDCl3) 0.20-0.49 (m, 2H), 0.64-0.84 (m, 2H), 0.93 (d, J = 6.00 Hz, 6H), 1.47-1.67 (m, 4H), 1.86-2.37 (m, 4H), 2.14 (s, 3H), 267-3.01 (m, 4H), 3.50-3.82 (m, 1H), 6.76-7.09 (m, 2H), 7.68-7.90 (m, 1H), 8.21-8.46 (m, 1H) | 548 | 546 |

TABLE 114

| | | | MS | |
|---|---|---|---|---|
| Ex- | | | M + H | M − H |
| am- | | | or | or |
| ple | Chemical structure | NMR | M − Na + H | M − Na − H |
| E-11 | | (400 MHz. DMSO-d6) 0.61-0.71 (m, 1H), 0.73- 0.81 (m, 1H), 0.90-0.95 (m, 2H), 0.99 (d, J = 6.62 Hz, 6H), 1,79-1.86 (m, 1H), 1.85-1.94 (m, 4H), 2.13-2.24 (m, 1H), 2.26 (s, 3H), 2.79-2.91 (m, 2H), 2.95 (d, J = 7.06 Hz, 2H), 3.49-3.56 (m, 1H), 7.07 (d, J = 8.38 Hz, 1H), | 501 | 499 |
| | | 7.26 (s,1H), 7.41 (d, J = | | |
| | | 8.16 Hz, 1H), 9.73 (s, 1H) | | |
| E-12 | | (400 MHz, DMSO-D6) 0.91-0.96 (m, 6H), 1.79- 1.90 (m, 4H), 1.99-2.08 (m, 1H), 2.25-2.28 (m, 3H), 2.76-2.80 (m, 2H), 2.86-291 (m, 2H), 3.49- 3.56 (m, 1H), 6.88 (s, 1H), 6.89-6.94 (m, 1H), 7.00- 7.06 (m, 1H), 7.56-7.62 (m, 1H), 10.07 (br s, 1H). | 512 | 510 |
| E-13 | | (400 MHz, DMSO-D6) 0.91 (d, J = 25.14 Hz, 6H), 1.54-1.64 (m, 3H), 1.81- 1.91 (m, 4H), 2.24-2.28 (m, 3H), 2.86-2.93 (m, 4H), 3.49-3.56 (m, 1H), 6.88 (s, 1H), 6.89-6.94 (m, 1H), 7.01-7.06 (m, 1H), 7.54-7.62 (m, 1H), 10.04 (br s, 1H). | 526 | 524 |
| E-14 | | (400 MHz, CDCl3) 0.44- 0.57 (m, 1H), 0.59-0.72 (m, 1H), 0.80-0.93 (m, 2H), 0.96 (d, J = 6.40 Hz, 6H), 1.53-1.66 (m, 1H), 1.87-2.27 (m, 5H), 2.11 (s, 3H), 2.75-2.86 (m, 1H), 2.87-3.01 (m, 1H), 2.96 (d, J = 6.80 Hz, 2H), 3.53-3.66 (m, 1H), 6.78 (d, J = 8.00 Hz, 1H), 6.92 | 517 | 515 |
| | | (s, 1H), 7.72 (d, J = 8.00 | | |
| | | Hz, 1H), 8.40 (brs, 1H) | | |
| E-15 | | (400 MHz, DMSO-d6) 0.63-0.70 (m, 1H), 0.79- 0.85 (m, 1H), 0.93 (d, J = 6.62 Hz, 6H), 0.94-0.99 (m, 2H), 1.76-1.94 (m, 6H), 2.25 (s, 3H), 2.73- 2.88 (m, 2H), 2.81 (d, J = 7.06 Hz, 2H), 3.47-3.54 (m, 1H), 7.06 (d, J = 7.94 Hz, 1H), 7.25 (s, 1H), | 516 | 514 |
| | | 7.41 (d, J = 8.16 Hz, 1H), | | |
| | | 7.80 (s, 1H), 9.81 (s, 1H) | | |

TABLE 115

| | | | MS | |
|---|---|---|---|---|
| Ex- | | | M + H | M − H |
| am- | | | or | or |
| ple | Chemical structure | NMR | M − Na + H | M − Na − H |
| E-16 | | (400 MHz, DMSO-D6) 0.92 (d, J = 16.92 Hz, 6H), 1.53-1.65 (m; 4H), 1.76-1.99 (m, 4H), 2.25-2.28 (m, 3H), 2.84-2.93 (m, 3H), 3.47-3.60 (m, 1H), 6.87 (s, 1H), 7.04-7.11 (m, 1H), 7.27 (br s, 1H), 7.39-7.43 (m, 1H), 9.89 (br s, 1H). | 542 | 540 |
| E-17 | | (400 MHz, DMSO-d6) 0.47-0.56 (m, 1H), 0.63-0.72 (m, 1H), 0.86 (s, 9H), 0.91-0.94 (m, 2H), 1.37 (d, J = 6.62 Hz, 2H), 1.70-1.77 (m, 1H), 1.81-1.95 (m, 6H), 2.26 (s, 3H), 2.38-2.47 (m, 1H), 2.48-2.56 (m, 2H), 2.76-2.91 (m, 2H), | 568 | 566 |
| | | 3.49-3.57 (m, 2H), | | |
| | | 3.56-3.65 (m, 2H), 6.77 | | |
| | | (s, 1H), 7.07 (d, J = | | |
| | | 7.94 Hz, 1H), 7.27 (s, | | |
| | | 1H), 7.41 (d, J = 8.16 | | |
| | | Hz, 1H), 9.75 (s, 1H) | | |
| E-18 | | (400 MHz, DMSO-d6) 0.66-0.73 (m, 1H), 0.76-0.83 (m, 1H), 0.90-0.94 (m, 2H), 0.93 (dd, J = 6.62 1.54 Hz, 6H), 1.78- 1.94 (m, 5H), 194-2.04 (m, 1H), 2.26 (s, 3H), 2.65 (d, J = 7.06 Hz, 2H), 2.76-2.90 (m, 2H), 3.47-3.54 (m, | 500 | 498 |
| | | 1H), 7.07 (d, J = 7.94 | | |
| | | Hz, 1H), 7.19 (s, 1H), | | |
| | | 7.26 (s, 1H), 7.41 (d, | | |
| | | J = 8.16 Hz, 1H), 9.74 | | |
| | | (s, 1H) | | |
| E-19 | | (400 MHz, DMSO-D6) 0.19-0.27 (m, 1H), 0.39-0.48 (m, 1H), 0.81-0.90 (m, 2H), 0.93 (s, 9H), 1.53-1.66 (m, 1H), 1.73-1.84 (m, 2H), 1.85-2.02 (m, 5H), 2.22-2.28 (m, 3H), 2.72-2.90 (m, 2H), 3.47-3.58 (m, 1H), 4.21-4.37 (m, 2H), 6.87-6.97 (m, 1H), | 579 | 577 |
| | | 6.98-7.07 (m, 1H), | | |
| | | 7.52-7.62 (m, 1H), | | |
| | | 8.48-8.57 (m, 1H), | | |
| | | 9.87-9.94 (m, 1H). | | |
| E-20 | | (400 MHz, DMSO-d6) 0.48-0.57 (m, 1H), 0.65-0.74 (m, 1H), 0.91-0.99 (m, 2H), 1.04 (d, J = 6.84 Hz, 6H), 1.75-1.82 (m, 1H), 1.84-1.95 (m, 4H), 2.26 (s, 3H), 2.58-2.72 (m, 1H, 2.79-2.93 (m, 2H), 3.52-3.59 (m, 1H, 7.08 (d, J = 8.16 Hz, 1H), | 536 | 534 |
| | | 7.27 (s, 1H), 7.42 (d, | | |
| | | J = 8.16 Hz, 1H), 7.46 | | |
| | | (s, 1H), 9.75 (s, 1H) | | |

TABLE 116

| | | | MS | |
|---|---|---|---|---|
| Ex- | | | M + H | M − H |
| am- | | | or | or |
| ple | Chemical structure | NMR | M − Na + H | M − Na − H |
| E-21 | | (400 MHz, DMSO-d6) 0.45-0.51 (m, 1H), 0.64-0.70 (m, 1H), 0.92 (d, J = 6.40 Hz, 6H), 0.93-0.96 (m, 2H), 1.39-1.47 (m, 2H), 1.55-1.62 (m, 3H), 1.67-1.77 (m, 3H), 1.84 (t, J = 7.28 Hz, 2H), 2.26 (s, 3H), 2.78-2.92 (m, 4H), 3.47-3.55 (m, 1H), 6.75 (s, 1H), 7.08 (d, J = 8.38 Hz, 1H), | 528 | 526 |
| | | 7.27 (s, 1H), 7.42 (d, J = | | |
| | | 8.16 Hz, 1H), 9.60 | | |
| | | (s, 1H) | | |
| E-22 | | (400 MHz, CDCl3) 0.34-0.59 (m, 2H), 0.70- 0.86 (m, 2H), 0.91 (s, 9H), 1.46-1.62 (m, 3H), 1.84-2.26 (m, 4H), 2.10 (s, 3H), 2.44-2.83 (m, 3H), 2.83-2.99 (m, 1H), 3.46-3.67 (m, 1H), 6.23 (s, 1H), 6.79 (d, J = 8.00 Hz, 1H), 6.93 (s, 1H), 7.71 (d, J = 8.00 Hz, 1H), 8.40 (s, 1H) | 528 | 526 |
| E-23 | | (400 MHz, CDCl3) 0.34-0.58 (m, 2H), 0.72-0.93 (m, 4H), 0.85 (d, J = 8.00 Hz, 6H), 1.00-1.11 (m, 2H), 1.45- 1.60 (m, 1H), 1.55 (d, J = 6.80 Hz, 2H), 1.66- 1.81 (m, 1H), 1.84-2.26 (m, 4H), 2.10 (s, 3H), 2.69-2.97 (m, 2H), 3.47- 3.66 (m, 1H), 6.20 (s, 1H), 6.78 (d, J = 8.00 Hz, 1H), 6.93 (s, 1H), | 540 | 538 |
| | | 7.72 (d, J = 8.00 Hz, | | |
| | | 1H), 8.38 (s, 1H) | | |
| E-24 | | (400 MHz, CDCl3) 0.34-0.61 (m, 2H), 0.67-0.90 (m, 2H), 1.42- 1.81 (m, 7H), 1.84-2.27 (m, 6H), 2.10 (s, 3H), 2.67-2.99 (m, 2H), 3.06- 3.24 (m, 1H), 3.44-3.77 (m, 1H), 6.22 (s, 1H), 6.67-6.85 (m, 1H), 6.93 (s, 1H), 7.60-7.84 (m, 1H), 8.26-8.53 (m, 1H) | 512 | 510 |
| E-25 | | (400 MHz, CDCl3) 0.10-0.22 (m, 1H), 0.22-0.33 (m, 1H), 0.62-0.78 (m, 2H), 0.88 (d, J = 6.40 Hz, 6H), 1.36-1.48 (m, 1H), 1.78- 1.91 (m, 1H), 1.91-2.33 (m, 4H), 2.09 (s, 3H), 2.41 (d, J = 6.80 Hz, 2H), 2.60-3.05 (m, 2H), 3.50-3.81 (m, 1H), | 513 | 511 |
| | | 3.72 (s, 3H), 6.21 (s, | | |
| | | 1H), 6.77 (d, J = 7.60 | | |
| | | Hz, 1H), 6.93 (s, 1H), | | |
| | | 7.74 (d, J = 7.60 Hz, | | |
| | | 1H), 8.33 (s, 1H) | | |

TABLE 117

| | | | MS | |
|---|---|---|---|---|
| Ex- | | | M + H | M − H |
| am- | | | or | or |
| ple | Chemical structure | NMR | M − Na + H | M − Na − H |
| E-26 | | (400 MHz, CDCl3) 0.24- 0.38 (m, 1H), 0.39-0.50 (m, 1H), 0.68-0.82 (m, 2H), 0.91 (d, J = 6.40 Hz, 6H), 1.42-1.55 (m, 1H), 1.76-1.92 (m, 2H), 1.93-2.19 (m, 3H), 2.12 (s, 3H), 2.40 (d, J = 7.20 Hz, 2H), 2.59-2.73 (m, 1H), 2.79-2.92 (m, 1H), 3.44-3.59 (m, 1H), 3.74 | 513 | 511 |
| | | (s, 3H), 6.33 (s, 1H), | | |
| | | 6.81 (d, J = 8.00 Hz, | | |
| | | 1H), 6.98 (s, 1H), 7.67 | | |
| | | (d, J = 8.00 Hz, 1H), | | |
| | | 8.60 (s, 1H) | | |
| E-27 | | (400 MHz, DMSO-d6) 0.60-0.69 (m, 1H), 0.77- 0.86 (m, 1H), 0.93 (d, J = 6.62 Hz, 6H), 0.94- 0.98 (m, 2H), 1.73-1.80 (m, 1H), 1.82-1.92 (m, 5H), 2.26 (s, 3H), 2.37 (s, 3H), 2.72 (d, J = 7.06 Hz, 2H), 2.75-2.90 (m, 2H), 3.47-3.54 (m, 1H), 7.07 (d, J = 8.16 Hz, | 530 | 528 |
| | | 1H), 7.26 (s, 1H), 7.41 | | |
| | | (d, J = 8.16 Hz, 1H), | | |
| | | 9.75 (s, 1H) | | |
| E-28 | | (400 MHz, DMSO-D6) 0.40-0.47 (m, 1H), 0.56-0.64 (m, 1H), 0.81- 0.91 (m, 2H), 0.92 (dd, J = 6.75, 1.16 Hz, 6H), 1.62 (td, J = 9.01, 4.73 Hz, 1H), 1.84-1.93 (m, 4H), 1.97 (td, J = 13.61, 6.90 Hz, 1H), 2.24 (s, 3H), 2.58 (d, J = 6.98 Hz, 2H), 2.63-2.83 (m, | 499 | 497 |
| | | 2H), 3.50 (d, J = 6.75 | | |
| | | Hz, 1H), 6.36 (d, J = 3.26 | | |
| | | Hz, 1H), 6.89 (d, J = 3.49 | | |
| | | Hz, 1H), 7.04 (d, J = 7.44 | | |
| | | Hz, 1H), 7.23 (s, 1H), | | |
| | | 7.42 (d, J = 8.37 Hz, 1H), | | |
| | | 9.77 (s, 1H) | | |
| E-29 | | (400 MHz, CDCl3) 0.24- 0.50 (m, 2H), 0.62-0.80 (m, 2H), 0.90 (d, J = 6.40 Hz, 6H), 1.47-1.61 (m, 1H), 1.75-2.28 (m, 5H), 1.90 (s, 3H), 2.11 (s, 3H), 2.52 (d, J = 6.80 Hz, 2H), 2.71-3.00 (m, 2H), 3.49-3.69 (m, 1H), 6.74-6.89 (m, 1H), 6.95 (s, 1H), 7.69-7.87 (m, | 514 | 512 |
| | | 1H), 8.28-8.52 (m, 1H) | | |
| E-30 | | (400 MHz, CDCl3) 0.33- 0.59 (m, 2H), 0.69-0.91 (m, 2H), 1.10 (s, 3H), 1.20 (s, 3H), 1.45-1.58 (m, 1H), 1.84-2.34 (m, 8H), 2.19 (s, 3H), 2.65- 2.99 (m, 2H), 3.44-3.73 (m, 2H), 6.23 (s, 1H), 6.70-6.85 (m, 1H), 6.94 (s, 1H, 7.63-7.83 (m, 1H), 8.28-8.48 (m, 1H) | 526 | 524 |

TABLE 118

| | | | MS | |
|---|---|---|---|---|
| Ex- | | | M + H | M − H |
| am- | | | or | or |
| ple | Chemical structure | NMR | M − Na + H | M − Na − H |
| E-31 | | (400 MHz, DMSO-D6) 0.51-0.56 (m, 1H), 0.66-0.71 (m, 1H), 0.91-0.93 (m, 2H), 0.94 (s, 9H), 1.59-1.63 (m, 2H), 1.71-1.78 (m, 1H), 1.80-1.91 (m, 4H), 2.23 (s, 3H), 2.66-2.77 (m, 2H), 2.80-2.84 (m, 2H), 3.48-3.55 (m, 1H), 6.75 (s, 1H), 6.83 (d, | 512 | 510 |
| | | J = 7.50 Hz, 1H), 6.92- | | |
| | | 6.95 (m, 1H), 7.53 (dd, | | |
| | | J = 7.50, 7.50 Hz, 1H), | | |
| | | 9.94 (br s, 1H) | | |
| E-32 | | (400 MHz, DMSO-D6) 0.51-0.55 (m, 1H), 0.66-0.71 (m, 1H), 0.90-0.93 (m, 2H), 0.94 (s, 9H), 1.59-1.64 (m, 2H), 1.71-1.78 (m, 1H), 181-1.95 (m, 4H), 2.02 (s, 3H), 2.20 (s, 3H), 2.63-2.76 (m, 2H), 2.80-2.85 (m, 2H), 3.49-3.56 (m, 1H), | 508 | 506 |
| | | 6.75 (s, 1H), 6.85 (d, | | |
| | | J = 8.16 Hz, 1H), 6.91 | | |
| | | (s, 1H), 7.08 (d, J = 8.20 | | |
| | | Hz, 1H), 9.59 (br s, 1H) | | |
| E-33 | | (400 MHz, DMSO-d6) 0.46-0.55 (m, 1H), 0.62-0.72 (m, 1H), 0.89-0.98 (m, 2H), 0.94 (d, J = 6.51 Hz, 6H), 1.72-1.93 (m, 6H), 2.24 (s, 3H), 2.26-2.37 (m, 2H), 2.76-2.92 (m, 2H), 3.49-3.58 (m, 1H), 7.06 (d, J = 8.37 Hz, 1H), 7.25 (s, 1H), 7.40 (d, | 550 | 548 |
| | | J = 7.68 Hz, 1H), 7.48 | | |
| | | (s, 1H), 9.75 (s, 1H) | | |
| E-34 | | (400 MHz, DMSO-D6) 0.52-0.57 (m, 1H), 0.67-0.71 (m, 1H), 0.89-0.92 (m, 2H), 0.96 (s, 9H), 1.59-1.63 (m, 2H), 1.72-1.79 (m, 1H), 1.81-1.94 (m, 4H), 2.61- 2.76 (m, 2H), 2.80-2.85 (m, 2H), 3.50-3.57 (m, 1H), 6.74 (s, 1H), 6.99- 7.06 (m, 1H), 7.25-7.31 | 532 | 530 |
| | | (m, 1H), 7.54-7.58 (m, | | |
| | | 1H), 10.04 (br s, 1H) | | |
| E-35 | | (400 MHz, DMSO-d6) 0.45-0.54 (m, 1H), 0.62-0.71 (m, 1H), 0.93-1.00 (m, 2H), 0.98 (s, 9H), 1.74-1.82 (m, 1H), 1.84-1.96 (m, 4H), 2.26 (s, 3H), 2.39 (t, J = 19.19 Hz, 2H), 2.79- 2.93 (m, 2H), 3.52-3.57 (m, 1H), 7.07 (d, J = 8.16 Hz, 1H), 7.26 (s, | 564 | 562 |
| | | 1H), 7.41 (d, J = 8.16 | | |
| | | Hz, 1H), 7.49 (s, 1H), | | |
| | | 9.74 (s, 1H) | | |

TABLE 119

| | | | MS | |
|---|---|---|---|---|
| Ex- | | | M + H | M − H |
| am- | | | or | or |
| ple | Chemical structure | NMR | M − Na + H | M − Na − H |
| E-36 | | (400 MHz, DMSO-D6) 0.40- 0.48 (m, 1H), 0.60-0.68 (m, 1H), 0.90 (dd, J = 8.49, 1.98 Hz, 2H), 0.96 (d, J = 6.75 Hz, 6H), 1.72-1.76 (m, 1H), 1.79-1.93 (m, 4H), 2.06-2.13 (m, 1H), 2.26 (s, 3H), 2.71- 2.90 (m, 2H), 2.92 (d, J = 7.21 Hz, 2H), 3.51 (t, J = 6.16 Hz, 1H), 7.07 (d, J = 8.14 Hz, 1H), 7.27 (s, 1H), | 516 | 514 |
| | | 7.43 (d, J = 8.14 Hz, 1H), | | |
| | | 8.03 (s, 1H), 9.79 (s, 1H) | | |
| E-37 | | (400 MHz, DMSO-D6) 0.51- 0.56 (m, 1H), 0.70-0.72 (m, 1H), 0.89-0.93 (m, 2H), 0.94 (s, 9H), 1.59-1.64 (m, 2H), 1.72-1.79 (m, 1H), 1.83-1.96 (m, 4H), 2.04 (s, 3H), 2.60- 2.75 (m, 2H), 2.80-2.85 (m, 2H), 3.49-3.57 (m, 1H), 6.74 (s, 1H), 6.82-6.86 (m, 1H), 6.92 (dd, J = 9.67, 2.01 Hz, 1H), 7.17-7.21 (m, 1H), | 512 | 510 |
| | | 9.72 (br s, 1H) | | |
| E-38 | | (400 MHz, DMSO-D6) 0.65- 0.70 (m, 1H), 0.90-0.93 (m, 2H), 0.94 (s, 9H), 1.28-1.57 (m, 1H), 1.59-1.63 (m, 2H), 1.71-1.78 (m, 1H), 1.83-1.95 (m, 4H), 2.08 (s, 3H), 2.67- 2.79 (m, 2H), 2.81-2.85 (m, 2H), 3.49-3.56 (m, 1H), 6.75 (s, 1H, 7.10 (dd, J = 8.66, 2.42 Hz, 1H), 7.18 (s, 1H), 7.31 (d, J = 8.66 Hz, 1H), | 528 | 526 |
| | | 9.87 (br s, 1H) | | |
| E-39 | | (400 MHz, DMSO-D6) 0.50- 0.54 (m, 1H), 0.66-0.70 (m, 1H), 0.91-0.99 (m, 2H), 1.49- 1.67 (m, 6H), 1.71-1.98 (m, 7H), 2.26 (s, 3H), 2.71-3.09 (m, 3H), 3.54 (dd, J = 14.45, 6.51 Hz, 1H), 7.08 (d, J = 8.38 Hz, 1H), 7.27 (s, 1H), 7.41 (d, J = 8.16 Hz, 1H), 7.48 (s, 1H), 9.79 (s, 1H) | 562 | 560 |
| E-40 | | (400 MHz, DMSO-d6) 0.48- 0.55 (m, 1H), 0.64-0.70 (m, 1H), 0.91-0.94 (m, 2H), 1.08 (s, 9H), 1.74-1.81 (m, 1H), 1.81-1.93 (m, 4H), 2.24 (s, 3H), 2.77-2.90 (m, 2H), 3.50- 3.57 (m, 1H), 7.06 (d, J = 7.44 Hz, 1H), 7.25 (s, 1H), 7.38 (s, 1H), 7.40 (d, J = 8.14 Hz, 1H), 9.78 (s, 1H) | 550 | 548 |

TABLE 120

| | | | MS | |
|---|---|---|---|---|
| Ex- | | | M + H | M − H |
| am- | | | or | or |
| ple | Chemical structure | NMR | M − Na + H | M − Na − H |
| E-41 | | (400 MHz, CDCl3) 0.22-0.34 (m, 1H), 0.39-0.53 (m, 1H), 0.77-0.93 (m, 2H), 0.97 (d, J = 6.80 Hz, 6H), 1.34-1.48 (m, 1H), 1.86-2.34 (m, 5H), 2.11 (s, 3H), 2.72-3.05 (m, 2H), 2.83 (d, J = 8.00 Hz, 2H), 3.52- 3.68 (m, 1H), 6.77 (d, J = 8.00 Hz, 1H), 6.94 (s, 1H), 7.77 (d, J = 8.00 Hz, 1H), 7.94 (s, 1H), 8.33 (brs, 1H) | 516 | 514 |
| E-42 | | (400 MHz, CDCl3) 0.36-0.63 (m, 2H), 0.71-0.91 (m, 2H), 1.34 (s, 3H), 1.46-1.59 (m, 1H), 1.59-1.82 (m, 6H), 1.87- 2.30 (m, 6H), 2.12 (s, 3H), 2.72- 3.00 (m, 2H), 3.50-3.71 (m, 1H), 6.25 (s, 1H), 6.79 (d, J = 7.20 Hz, 1H), 6.94 (s, 1H), 7.66-7.84 (m, 1H), 8.29-8.52 (m, 1H) | 526 | 524 |
| E-43 | | (400 MHz, DMSO-d6) 0.51- 0.57 (m, 1H), 0.66-0.72 (m, 1H), 0.91-0.94 (m, 2H), 1.36 (s, 9H), 1.71-1.78 (m, 1H), 1.83-1.94 (m, 4H), 2.26 (s, 3H), 2.76-2.90 (m, 2H), 3.49- 3.56 (m, 1H), 6.71 (s, 1H), 7.07 (d, J = 8.16 Hz, 1H), 7.28 (s, 1H), 7.41 (d, J = 8.38 Hz, 1H), 9.77 (s, 1H) | 500 | 498 |
| E-44 | | (400 MHz, DMSO-D6) δ: 0.48- 0.53 (1H, m), 0.63-0.69 (1H, m), 0.91 (2H, dd, J = 8.55, 1.85 Hz), 1.10 (6H, s), 1.26 (6H, s), 1.71-1.77 (2H, m), 1.82-1.92 (4H, m), 2.26 (3H, s), 2.83 (2H, ddd, J = 32.19, 15.43, 7.92 Hz), 3.53 (1H, t, J = 7.05 Hz), 6.64 (1H, s), 7.07 (1H, d, J = 7.86 Hz), 7.26 (1H, s), 7.42 (1H, d, J = 8.09 Hz), 9.76 (1H, br s). | 540 | 538 |
| E-45 | | (400 MHz, CDCl3) 0.30-0.64 (m, 2H), 0.64-1.02 (m, 2H), 0.92 (s, 9H), 1.40-1.67 (m, 3H), 1.78-2.42 (m, 4H), 2.03 (s, 3H), 2.56-3.03 (m, 4H), 3.38-3.78 (m, 1H), 6.24 (s, 1H), 6.78-7.02 (m, 1H), 7.59- 7.90 (m, 1H), 8.29-8.56 (m, 1H) | 546 | 544 |

TABLE 121

| | | | MS | |
|---|---|---|---|---|
| | | | M + H | M − H |
| | | | or | or |
| Example | Chemical structure | NMR | M − Na + H | M − Na − H |
| E-46 | | (400 MHz, DMSO-d6) 0.51- 0.57 (m, 1H), 0.67-0.73 (m, 1H), 0.92-0.98 (m, 2H), 1.10 (s, 9H), 1.76-1.83 (m, 1H), 1.83-1.92 (m, 4H), 2.26 (s, 3H), 2.79-2.93 (m, 2H), 3.51- 3.58 (m, 1H), 6.91 (d, J = 7.94 Hz, 1H), 7.02 (d, J = 11.69 Hz, 1H), 7.40 (s, 1H), 7.58 (t, J = 8.27 Hz, 1H), 9.94 (s, 1H) | 534 | 532 |
| E-47 | | (400 MHz, DMSO-D6) 0.59- 0.55 (m, 1H), 0.73-0.69 (m, 1H), 0.90-0.94 (m, 2H), 1.73- 1.80 (m, 1H), 1.80-1.95 (m, 4H), 2.21 (s, 3H), 2.61-2.75 (m, 2H), 2.87-3.00 (m, 2H), 3.06-3.18 (m, 2H), 3.50-3.56 (m, 1H), 3.69-3.77 (m, 1H), 6.94 (d, J = 8.16 Hz, 1H), 6.98 (s, 1H), 7.14 (s, 1H), 7.41 (d, J = 8.16 Hz, 1H), 9.77 (br s, 1H) | 534 | 532 |
| E-48 | | (400 MHz, CDCl3) 0.30-0.61 (m, 2H), 0.74-0.95 (m, 2H), 1.06 (s, 9H), 1.45-1.62 (m, 1H), 1.89-2.28 (m, 4H), 2.75- 3.04 (m, 2H), 3.47-3.76 (m, 1H), 6.62-6.79 (m, 2H), 6.79- 6.94 (m, 1H), 7.67-7.89 (m, 1H), 8.42-8.69 (m, 1H) | 554 | 552 |
| E-49 | | (400 MHz, CDCl3) 0.25-0.63 (m, 2H), 0.69-0.92 (m, 2H), 1.06 (s, 9H), 1.42-1.62 (m, 1H), 1.78-2.32 (m, 4H), 2.47- 3.07 (m, 2H), 3.43-3.76 (m, 1H), 6.60-6.99 (m, 3H), 7.70- 7.95 (m, 1H), 8.76-9.21 (m, 1H) | 554 | 552 |
| E-50 | | (400 MHz, DMSO-d6) 0.48- 0.55 (m, 1H), 0.64-0.71 (m, 1H), 0.90-0.98 (m, 2H), 1.70- 1.77 (m, 1H), 1.82-1.95 (m, 4H), 2.26 (s, 3H), 2.74-2.91 (m, 4H), 3.15 (t, J = 7.61 Hz, 2H), 3.49-3.56 (m, 1H), 6.91 (s, 1H), 7.07 (d, J = 7.72 Hz, 1H), 7.26 (s, 1H), 7.41 (d, J = 8.16 Hz, 1H), 9.75 (s, 1H) | 540 | 538 |

TABLE 122

| | | | MS | |
|---|---|---|---|---|
| Ex- | | | M + H | M − H |
| am- | | | or | or |
| ple | Chemical structure | NMR | M − Na + H | M − Na − H |
| E-51 | | (400 MHz, DMSO-d6) 0.49- 0.56 (m, 1H), 0.65-0.73 (m, 3H), 0.94-0.97 (m, 2H), 1.00- 1.02 (m, 2H), 1.16 (s, 3H), 1.76- 1.83 (m, 1H), 1.83-1.95 (m, 4H), 2.26 (s, 3H), 2.79-2.93 (m, 2H), 3.52-3.59 (m, 1H), 7.08 (d, J = 8.16 Hz, 1H), 7.27 (s, 1H), 7.42 (d, J = 8.38 Hz, 1H), 7.46 (s, 1H), 9.76 (s, 1H) | 548 | 546 |
| E-52 | | (400 MHz, DMSO-d6) 0.38- 0.51 (m, 1H), 0.59-0.70 (m, 1H), 0.74-0.87 (m, 2H), 1.09 (s, 9H), 1.61-2.00 (m, 5H), 2.23 (s, 3H), 2.68-2.90 (m, 2H), 3.57-3.73 (m, 1H), 7.02- 7.07 (m, 1H), 7.21-7.26 (m, 1H), 7.32 (s, 1H), 7.37 (d, J = 8.00 Hz, 1H), 9.71 (brs, 1H) | 550 | 548 |
| E-53 | | (400 MHz, DMSO-d6) 0.66- 0.70 (m, 2H), 0.88-0.92 (m, 2H), 1.08 (s, 9H), 1.78-1.85 (m, 1H), 2.18-2.24 (m, 1H), 2.24 (s, 3H), 2.37 (dd, J = 14.91, 5.66 Hz, 1H), 2.77-2.83 (m, 1H), 2.89-2.94 (m, 1H), 3.75- 3.82 (m, 1H), 7.06 (d, J = 8.55 Hz, 1H), 7.25 (s, 1H), 7.36 (s, 1H), 7.45 (d, J = 8.09 Hz, 1H), 9.94 (s, 1H) | 536 | 534 |
| E-54 | | (400 MHz, DMSO-D6) 0.55- 0.59 (m, 1H), 0.69-0.74 (m, 1H), 0.92 (dd, J = 8.38, 2.21 Hz, 2H), 1.63 (s, 6H), 1.76-1.95 (m, 5H), 2.19 (s, 3H), 2.55- 2.68 (m, 2H), 3.51-3.58 (m, 1H), 6.89 (d, J = 8.20 Hz, 1H), 7.09 (s, 1H), 7.17 (s, 1H), 7.41 (d, J = 8.16 Hz, 1H), 9.76 (br s, 1H) | 554 | 552 |
| E-55 | | (400 MHz, DMSO-D6) δ: 0.52- 0.56 (1H, m), 0.57-0.71 (1H, m), 0.96 (2H, dq, J = 8.73, 2.08 Hz), 1.04 (6H, d, J = 6.94 Hz), 1.77-1.90 (5H, m), 2.64 (1H, ddd, J = 28.32, 13.76, 6.59 Hz), 2.89 (2H, ddd, J = 27.63, 15.26, 7.63 Hz), 3.55 (1H, t, J = 7.28 Hz), 7.20 (1H, d, J = 8.29 Hz), 7.43 (1H, dd, J = 10.52, 2.43 Hz), 7.46 (1H, s), | 540 | 538 |
| | | 7.81 (1H, t, J = 8.67 Hz), 10.33 | | |
| | | (1H, br s). | | |

TABLE 123

| | | | MS | |
|---|---|---|---|---|
| Ex- | | | M + H | M − H |
| am- | | | or | or |
| ple | Chemical structure | NMR | M − Na + H | M − Na − H |
| E-56 | | (400 MHz, DMSO-d6) 0.48- 0.58 (m, 1H), 0.62-0.72 (m, 1H), 0.87-1.02 (m, 2H), 0.97 (s, 9H), 1.69-1.80 (m, 1H), 1.80-1.95 (m, 4H), 2.77 (s, 2H), 2.81 (dd, J = 15.20, 6.40 Hz, 1H), 2.89 (dd, J = 15.20, 9.20 Hz, 1H), 3.48-3.58 (m, 1H), 6.74 (s, 1H), 7.13-7.21 (m, 1H), 7.35-7.44 (m, 1H), 7.81 (t, J = 8.40 Hz, 1H), 10.27 (brs, 1H) | 518 | 516 |
| E-57 | | (400 MHz, DMSO-d6) 0.47-0.57 (m, 1H), 0.61-0.72 (m, 1H), 0.87- 1.02 (m, 2H), 0.97 (s, 9H), 1.69- 1.80 (m, 1H), 1.81-1.99 (m, 4H), 2.77 (s, 2H), 2.79 (dd, J = 14.80, 6.80 Hz, 1H), 2.87 (dd, J = 14.80, 8.00 Hz, 1H), 3.49-3.60 (m, 1H), 6.75 (s, 1H), 7.16 (ddd, J = 8.80, 8.80, 2.80 Hz, 1H), 7.43 (dd, J = 8.80, 2.80 Hz, 1H), 7.53 (dd, J = 8.80, 6.00 Hz, 1H), 10.04 (brs, 1H) | 518 | 516 |
| E-58 | | (400 MHz, DMSO-d6) 0.43-0.55 (m, 1H), 0.59-0.72 (m, 1H), 0.85- 1.02 (m, 2H), 0.95 (s, 9H), 1.67- 1.78 (m, 1H), 1.78-1.98 (m, 4H), 2.76 (s, 2H), 2.82 (dd, J = 15.20, 6.80 Hz, 1H), 2.90 (dd, J = 15.20, 8.40 Hz, 1H), 3.47-3.59 (m, 1H), 6.73 (s, 1H), 7.34 (dd, J = 8.80, 2.40 Hz, 1H), 7.59 (d, J = 2.40 Hz, 1H), 7.61 (d, J = 8.80 Hz, 1H), 10.03 (brs, 1H) | 534 | 532 |
| E-59 | | (400 MHz, DMSO-d6) 0.50-0.57 (m, 1H), 0.64-0.71 (m, 1H), 0.89- 0.97 (m, 2H), 1.63-1.78 (m, 8H), 1.83-1.92 (m, 4H), 2.03-2.12 (m, 2H), 2.81-2.94 (m, 2H), 3.49-3.56 (m, 1H), 6.74 (s, 1H), 7.20 (d, J = 8.82 Hz, 1H), 7.43 (dd, J = 10.59, 2.21 Hz, 1H), 7.81 (t, J = 8.60 Hz, 1H), 10.27 (s, 1H) | 516 | 514 |
| E-60 | | (400 MHz, DMSO-d6) 0.48-0.56 (m, 1H), 0.64-0.71 (m, 1H), 0.89- 0.96 (m, 2H), 1.62-1.78 (m, 8H), 1.83-1.94 (m, 4H), 2.03-2.12 (m, 2H), 2.77-2.91 (m, 2H), 3.50-3.56 (m, 1H), 6.75 (s, 1H), 7.17 (td, J = 8.66, 2.79 Hz, 1H), 7.44 (dd, J = 8.71, 2.76 Hz, 1H), 7.52 (dd, J = 8.82, 5.95 Hz, 1H), 10.01 (s, 1H) | 516 | 514 |

TABLE 124

| | | | MS | |
|---|---|---|---|---|
| | | | M + H | M − H |
| Ex- | | | or | or |
| am- | | | M − Na + | M − Na − |
| ple | Chemical structure | NMR | H | H |
| E-61 | | (400 MHz, DMSO-D6) δ: 0.49- 0.54 (1H, m), 0.65-0.69 (1H, m), 0.94 (2H, dd, J = 8.32, 1.85 Hz), 1.02 (6H, d, J = 6.94 Hz), 1.74-1.94 (5H, m), 2.63 (1H, ddd, J = 28.96, 14.51, 6.99 Hz), 2.84 (2H, ddd, J = 29.71, 15.14, 7.17 Hz), 3.54 (1H, t, J = 7.05 Hz), 7.15 (1H, td, J = 8.67, 3.01 Hz), 7.42 (1H, dd, J = 8.67, 2.89 Hz), 7.45 (1H, s), | 540 | 538 |
| | | 7.52 (1H, dd, J = 9.02, 5.78 | | |
| | | Hz), 10.04 (1H, br s). | | |
| E-62 | | (400 MHz, DMSO-D6) δ: 1.10 (9H, s), 1.79-1.95 (4H, m), 2.91 (2H, dd, J = 7.39, 2.98 Hz), 3.61 (1H, t, J = 6.51 Hz), 7.20 (1H, dq, J = 8.44, 1.14 Hz), 7.44 (1H, dd, J = 10.59, 2.43 Hz), 7.49 (1H, t, J = 52.82 Hz), 7.56 (1H, s), 7.83 (1H, t, J = 8.60 Hz), 10.35 (1H, br s). | 564 | 562 |
| E-63 | | (400 MHz, DMSO-D6) 0.37- 0.39 (m, 2H), 0.52-0.57 (m, 3H), 0.67-0.72 (m, 1H), 0.89- 0.94 (m, 2H), 1.05 (s, 3H), 1.74- 1.81 (m, 1H), 1.62-1.95 (m, 4H), 2.67-2.78 (m, 2H), 2.81 (s, 2H), 3.50-3.57 (m, 1H), 6.77 (s, 1H), 7.06 (d, J = 7.72 Hz, 1H), 7.32 (s, 1H), 7.53-7.58 (m, 1H), 10.04 (br s, 1H) | 516 | 514 |
| E-64 | | (400 MHz, DMSO-D6) 0.37- 0.39 (m, 2H), 0.51-0.56 (m, 3H), 0.67-0.71 (m, 1H), 0.92- 0.95 (m, 2H), 1.05 (s, 3H), 1.72- 1.79 (m, 1H), 1.82-1.90 (m, 4H), 2.75-2.90 (m, 4H), 3.49- 3.56 (m, 1H), 6.78 (s, 1H), 7.14- 7.18 (m, 1H), 7.35-7.40 (m, 1H), 7.81 (dd, J = 8.70, 8.70 Hz, 1H), 10.31 (br s, 1H) | 516 | 514 |
| E-65 | | (400 MHz, DMSO-d6) 0.43-0.56 (m, 1H), 0.60-0.71 (m, 1H), 0.82-0.71 (m, 2H), 0.95 (s, 9H), 1.67-1.79 (m, 1H), 1.79-1.95 (m, 4H), 2.24 (s, 3H), 2.75 (s, 2H), 2.81 (dd, J = 15.20, 6.00 Hz, 1H), 2.87 (dd, J = 15.20, 8.40 Hz, 1H), 3.45-3.57 (m, 1H), 6.73 (s, 1H), 6.90 (d, J = 8.40 Hz, 1H), 7.00 (d, J = 11.60 Hz, 1H), 7.57 (dd, J = 8.40, 8.00 Hz, 1H), 9.90 (brs, 1H) | 498 | 496 |

TABLE 125

| | | | MS | |
|---|---|---|---|---|
| Ex- | | | M + H | M − H |
| am- | | | or | or |
| ple | Chemical structure | NMR | M − Na + H | M − Na − H |
| E-66 | | (400 MHz, DMSO-D6) δ: 1.09 (9H, s), 1.81-1.92 (4H, m), 2.13 (3H, t, J = 19.30 Hz), 2.89 (2H, dd, J = 10.98, 9.13 Hz), 3.53 (1H, t, J = 7.40 Hz), 7.19 (1H, d, J = 8.09 Hz), 7.42 (1H, d, J = 11.56 Hz), 7.47 (1H, s), 7.81 (1H, t, J = 8.90 Hz), 10.31 (1H, br s). | 578 | 576 |
| E-67 | | (400 MHz, DMSO-d6) 0.51- 0.57 (m, 1H), 0.66-0.73 (m, 3H), 0.93-0.98 (m, 2H), 0.99- 1.02 (m, 2H), 1.16 (s, 3H), 1.76- 1.83 (m, 1H), 1.85-1.93 (m, 4H), 2.83-2.97 (m, 2H), 3.52- 3.58 (m, 1H), 7.20 (d, J = 8.60 Hz, 1H), 7.44 (d, J = 11.03 Hz, 1H), 7.46 (s, 1H), 7.82 (t, J = 8.7 Hz, 1H), 10.27 (s, 1H) | 552 | 550 |
| E-68 | | (400 MHz, DMSO-d6) 0.67-0.71 (m, 2H), 0.90-0.95 (m, 2H), 1.09 (s, 9H), 1.80-1.87 (m, 1H), 2.20 (dd, J = 14.78, 9.48 Hz, 1H), 2.37 (dd, J = 14.67, 5.18 Hz, 1H), 2.86 (dd, J = 15.22, 9.04 Hz, 1H), 2.96 (dd, J = 15.22, 5.29 Hz, 1H), 3.75-3.82 (m, 1H), 7.19 (d, J = 8.82 Hz, 1H), 7.38 (s, 1H), 7.43 (dd, J = 10.59, 2.43 Hz, 1H), 7.86 (t, J = 8.71 Hz, 1H), 10.59 (s, 1H) | 540 | 538 |
| E-69 | | (400 MHz, DMSO-d6) 0.52-0.58 (m, 1H), 0.66-0.71 (m, 1H), 0.89- 0.97 (m, 2H), 1.36 (s, 9H), 1.71-1.78 (m, 1H), 1.83-1.91 (m, 4H), 2.61- 2.94 (m, 2H), 3.49-3.55 (m, 1H), 6.71 (s, 1H), 7.20 (d, J = 8.60 Hz, 1H), 7.43 (dd, J = 10.59, 2.21 Hz, 1H), 7.81 (t, J = 8.60 Hz, 1H), 10.29 (s, 1H) | 504 | 502 |
| E-70 | | (400 MHz, DMSO-d6) 0.49-0.56 (m, 1H), 0.64-0.71 (m, 1H), 0.89- 0.96 (m, 2H), 1.60-1.79 (m, 8H), 1.82-1.95 (m, 4H), 2.02-2.13 (m, 2H), 2.80-2.94 (m, 2H), 3.49-3.56 (m, 1H), 6.74 (d, J = 0.66 Hz, 1H), 7.36 (dd, J = 8.60, 2.43 Hz, 1H), 7.60 (d, J = 0.88 Hz, 1H), 7.62 (d, J = 7.50 Hz, 1H), 10.05 (s, 1H) | 532 | 530 |

TABLE 126

| | | | MS | |
|---|---|---|---|---|
| | | | M + H | M − H |
| Ex- | | | or | or |
| am- | | | M − Na + | M − Na − |
| ple | Chemical structure | NMR | H | H |
| E-71 | | (400 MHz, DMSO-d6) 0.47- 0.61 (m, 1H), 0.63-0.76 (m, 1H), 0.86-1.00 (m, 2H), 1.38 (s, 3H), 1.62-1.96 (m, 11H), 1.96-2.11 (m, 2H), 2.84 (dd, J = 15.20, 6.40 Hz, 1H), 2.91 (dd, J = 15.20, 8.40 Hz, 1H), 3.47- 3.60 (m, 1H), 6.74 (s, 1H), 7.16- 7.23 (m, 1H), 7.43 (dd, J = 10.40, 2.40 Hz, 1H), 7.81 (dd, J = 8.80, 8.80 Hz, 1H), 10.26 (brs, 1H) | 530 | 528 |
| E-72 | | (400 MHz, DMSO-d6) 0.49-0.59 (m, 1H), 0.62-0.74 (m, 1H), 0.87- 0.98 (m, 2H), 1.39 (s, 3H), 1.65-1.97 (m, 11H), 1.97-2.11 (m, 2H), 2.80 (dd, J = 15.20, 6.40 Hz, 1H), 2.80 (dd, J = 15.20, 8.40 Hz, 1H), 3.48- 3.59 (m, 1H), 6.75 (s, 1H), 7.17 (ddd, J = 8.40, 8.40, 2.80 Hz, 1H), 7.44 (dd, J = 8.40, 2.80 Hz, 1H), 7.53 (dd, J = 9.20, 6.00 Hz, 1H), 10.03 (brs, 1H) | 530 | 528 |
| E-73 | | (400 MHz, DMSO-D6) 0.52-0.58 (m, 1H), 0.67-0.73 (m, 1H), 0.75-0.80 (m, 2H), 0.80-0.87 (m, 2H), 0.91-0.99 (m, 2H), 1.76-1.99 (m, 6H), 2.25 (s, 3H), 2.74-2.87 (m, 2H), 3.51-3.58 (m, 1H), 7.04 (d, J = 8.30 Hz, 1H), 7.24 (s, 1H), 7.41 (d, J = 8.26 Hz, 1H), 7.48 (s, 1H), 9.83 (br s, 1H) | 534 | 532 |
| E-74 | | (400 MHz, DMSO-D6) 0.42-0.51 (m, 1H), 0.58-0.68 (m, 1H), 0.76-0.85 (m, 2H), 0.97 (s, 9H), 1.51-1.71 (m, 1H), 1.72-1.98 (m, 4H), 2.76 (s, 2H), 2.77- 2.84 (m, 2H), 3.58-3.72 (m, 1H), 6.67 (s, 1H), 7.34 (dd, J = 8.60, 2.43 Hz, 1H), 7.56-7.62 (m, 2H), 9.98 (br s, 1H) | 534 | 532 |
| E-75 | | (400 MHz, DMSO-D6) 0.41-0.51 (m, 1H), 0.60-0.68 (m, 1H), 0.74-0.85 (m, 2H), 0.97 (s, 9H), 1.61-1.70 (m, 1H), 1.71-1.99 (m, 4H), 2.25 (s, 3H), 2.71-2.84 (m, 4H), 3.57-3.70 (m, 1H), 6.66 (s, 1H), 7.07 (d, J = 8.38 Hz, 1H), 7.26 (s, 1H), 7.38 (d, J = 8.38 Hz, 1H), 9.70 (br s, 1H) | 514 | 512 |

TABLE 127

| | | | MS | |
|---|---|---|---|---|
| | | | M + H | M − H |
| | | | or | or |
| Example | Chemical structure | NMR | M − Na + H | M − Na − H |
| E-76 | | (400 MHz, DMSO-D6) 0.42- 0.51 (m, 1H), 0.58-0.68 (m, 1H), 0.75-0.84 (m, 2H), 0.97 (s, 9H), 1.60-1.71 (m, 1H), 1.71-1.94 (m, 4H), 2.76 (s, 2H), 2.78-2.90 (m, 2H), 3.59- 3.69 (m, 1H), 6.67 (s, 1H), 7.20 (dt, J = 8.75, 2.40 Hz, 1H), 7.43 (dd, J = 10.59, 2.40 Hz, 1H), 7.80 (t, J = 8.75 Hz, 1H), 10.20 (br s, 1H) | 518 | 516 |

TABLE 128

| | | | | | Infor- |
|---|---|---|---|---|---|
| Ex- | | | | | mation |
| am- | | | MS | | of |
| ple | Chemical structure | NMR | M + H | M − H | structure |
| F-1 | | (400 MHz, CDCl3) 1.34 (t, J = 7.28 Hz, 3H), 3.12 (t, J = 6.62 Hz, 2H), 3.21 (dd, J = 15.22, 8.38 Hz, 2H), 3.34 (td, J = 14.45, 8.45 Hz, 4H), 3.58 (dd, J = 17.97, 9.15 Hz, 1H), 3.94 (q, J = 7.28 Hz, 2H), 7.16 (s, 4H), 7.40 (t, J = 7.83 Hz, 3H), 7.64 (d, J = 8.16 Hz, 1H), 7.79 (t, | 411 | 409 | |
| | | J = 10.03 Hz, 2H), 7.93 (d, | | | |
| | | J = 9.26 Hz, 1H), 9.60 (s, 1H) | | | |
| F2 | | (400 MHz, DMSO-D6) 1.29-1.36 (m, 6H), 2.89 (t, J = 7.61 Hz, 1H), 3.13 (t, J = 11.69 Hz, 1H), 3.32 (dt, J = 29.55, 9.70 Hz, 4H), 3.48 (d, J = 7.28 Hz, 1H), 3.79 (t, J = 8.60 Hz, 1H), 4.05 (dd, J = 13.67, 7.06 Hz, 2H), 7.17 (dd, J = 5.51, 3.31 Hz, 2H), 7.25 (d, J = 3.97 Hz, 2H), 7.44- | 425 | 423 | |
| | | 7.52 (m, 3H), 7.57 (d, J = 7.28 Hz, | | | |
| | | 1H), 7.74 (d, J = 7.94 Hz, 1H), 7.91 | | | |
| | | (d, J = 8.38 Hz, 2H), 10.02 (s, 1H) | | | |
| F-3 | | (400 MHz, CDCl3) 1.37 (t, J = 7.28 Hz, 3H), 2.21 (s, 3H), 3.09 (d, J = 6.62 Hz, 2H), 3.16 (d, J = 6.84 Hz, 2H), 3.28 (dd, J = 15.22, 8.60 Hz, 2H), 3.39 (dd, J = 15.22, 9.26 Hz, 2H), 3.62 (dd, J = 17.97, 8.93 Hz, 1H), 3.97 (q, J = 7.28 Hz, 2H), 7.04 (t, J = 8.05 Hz, 1H), 7.14- 7.21 (m, 5H), 7.46 (d, J = 7.94 Hz, 1H), 9.26 (s, 1H) | 409 | 407 | |

TABLE 129

| | | | | | Infor- |
|---|---|---|---|---|---|
| Ex- | | | MS | | mation |
| am- | | | M + | M − | of |
| ple | Chemical structure | NMR | H | H | structure |
| F-4 | | (400 MHz, CDCl3) 1.04 (dd, J = 9.15, 6.95 Hz, 2H), 1.23 (dd, J = 12.90, 6.95 Hz, 2H), 3.05 (td, J = 7.06, 3.68 Hz, 1H), 3.24 (t, J = 5.18 Hz, 4H), 3.35 (dd, J = 15.44, 8.60 Hz, 2H), 3.45 (dd, J = 15.33, 9.37 Hz, 2H), 3.82-3.91 (m, 1H), 7.17-7.25 (m, 4H), 7.42 (dd, J = 14.78, 7.06 Hz, 3H), 7.64 (d, J = 8.16 Hz, 1H), 7.80 (t, J = 4.52 Hz, 1H), 7.94 (dd, J = 13.12, 5.84 Hz, 2H), 9.14 (s, 1H) | 423 | 421 | |
| F-5 | | (400 MHz, CDCl3) 0.98 (t, J = 7.39 Hz, 3H), 3.15 (t, J = 6.18 Hz, 2H), 3.28 (dt, J = 12.28, 5.29 Hz, 4H), 3.41-3.49 (m, 2H), 3.59-3.68 (m, 1H), 3.85 (t, J = 7.83 Hz, 2H), 7.17-7.23 (m, 4H), 7.42 (t, J = 8.16 Hz, 3H), 7.65 (d, J = 8.38 Hz, 1H), 7.80 (t, J = 4.63 Hz, 1H), 7.87 (d, J = 7.50 Hz, 1H), 7.94 (t, J = 4.74 Hz, 1H), 9.24 (s, 1H) | 425 | 423 | |
| F-6 | | (400 MHz, CDCl3) 1.56 (d, J = 7.06 Hz, 6H), 3.30 (dd, J = 17.53, 10.48 Hz, 6H), 3.49 (dd, J = 15.44, 9.26 Hz, 2H), 3.68-3.77 (m, 1H), 4.48-4.55 (m, 1H), 7.21 (tt, J = 9.37, 3.23 Hz, 4H), 7.39-7.45 (m, 3H), 7.64 (d, J = 8.16 Hz, 1H), 7.81 (dd, J = 6.18, 3.53 Hz, 1H), 7.96 (d, J = 7.06 Hz, 2H), 9.92 (s, 1H) | 425 | 423 | |

TABLE 130

| | | | | | Infor- |
|---|---|---|---|---|---|
| Ex- | | | MS | | mation |
| am- | | | M + | M − | of |
| ple | Chemical structure | NMR | H | H | structure |
| F-7 | | (400 MHz, CDCl3) 1.39 (t, J = 7.60 Hz, 2H), 1.76 (ddd, J = 25.33, 11.42, 6.20 Hz, 4H), 2.52 (t, J = 6.03 Hz, 2H), 2.75 (t, J = 5.91 Hz, 2H), 3.11 (s, 4H), 3.32 (dd, J = 15.07, 8.35 Hz, 2H), 3.48 (dd, J = 15.31, 9.51 Hz, 2H), 3.67 (dt, J = 25.28, 7.54 Hz, 1H), 3.98 (q, J = 7.34 Hz, 3H), 6.89 (d, J = 7.65 Hz, 1H), 7.08 (t, J = 7.88 Hz, 1H), 7.18-7.26 (m, 4H), 7.59 (d, J = 7.88 Hz, 1H), 8.08 (s, 1H) | 415 | 413 | |
| F-8 | | (400 MHz, CDCl3) 3.08 (t, J = 6.51 Hz, 2H), 3.27 (ddd, J = 31.54, 15.44, 8.71 Hz, 6H), 3.49 (t, J = 13.45 Hz, 3H), 3.55-3.68 (m, 1H), 7.14 (dd, J = 27.79, 8.82 Hz, 4H), 7.42 (dt, J = 22.57, 10.97 Hz, 3H), 7.61 (t, J = 13.56 Hz, 1H), 7.78 (d, J = 7.72 Hz, 2H), 7.92 (d, J = 7.72 Hz, 1H), 9.59 (s, 1H) | 397 | 395 | |
| F-9 | | (400 MHz, DMSO-D6) 2.95 (d, J = 34.63 Hz, 4H), 3.23 (dd, J = 22.72, 13.45 Hz, 4H), 3.72 (s, 1H), 7.19 (t, J = 16.54 Hz, 4H), 7.42-7.56 (m, 3H), 7.61-7.68 (m, 1H), 7.71-7.79 (m, 1H), 7.90-7.96 (m, 1H), 8.00-8.09 (m, 1H), 9.84-10.04 (m, 1H), 13.21-13.61 (m, 1H) | 383 | 381 | |

TABLE 131

| | | | | | Infor- |
|---|---|---|---|---|---|
| Ex- | | | | | mation |
| am- | | | MS | | of |
| ple | Chemical structure | NMR | M + H | M − H | structure |
| F-10 | | (400 MHz, CDCl3) 1.40 (t, J = 7.30 Hz, 3H), 1.70-1.83 (m, 4H), 2.69-2.84 (m, 2H), 2.89 (t, J = 7.07 Hz, 2H), 3.04-3.10 (m, 2H), 3.33 (td, J = 10.03, 3.56 Hz, 2H), 3.45-3.52 (m, 2H), 3.62- 3.71 (m, 1H), 4.01 (q, J = 7.34 Hz, 2H), 5.14 (dd, J = 14.15, 6.03 Hz, 1H), 6.32 (d, J = 8.35 Hz, 1H), 7.06-7.26 (m, 8H) | 415 | 413 | |
| F-11 | | (400 MHz, CDCl3) 0.92 (t, J = 14.22 Hz, 6H), 2.02-2.09 (m, 1H), 3.15 (dd, J = 13.67, 7.06 Hz, 2H), 3.24-3.32 (m, 4H), 3.40-3.48 (m, 2H), 3.62 (dd, J = 17.75, 8.71 Hz, 1H), 3.72 (t, J = 8.49 Hz, 2H), 7.20 (td, J = 8.93, 4.04 Hz, 4H), 7.41-7.45 (m, 3H), 7.66 (t, J = 7.83 Hz, 1H), 7.80 (t, J = 4.63 Hz, 1H), 7.91 (dd, J = 13.78, 5.40 Hz, 2H), 9.17 (s, 1H) | 439 | 437 | |
| F-12 | | (400 MHz, CDCl3) 1.20 (t, J = 7.28 Hz, 3H), 2.96 (t, J = 8.05 Hz, 2H), 3.09-3.16 (m, 4H), 3.24 (t, J = 6.18 Hz, 2H), 3.73 (q, J = 7.35 Hz, 2H), 7.15-7.25 (m, 5H), 7.49 (tt, J = 19.74, 6.36 Hz, 3H), 7.66 (d, J = 7.94 Hz, 1H), 7.83 (d, J = 7.94 Hz, 1H), 7.97 (dd, J = 17.64, 8.16 Hz, 2H), 9.13 (s, 1H) | 399 | 397 | |

TABLE 132

| | | | | | Infor- |
|---|---|---|---|---|---|
| | | | | | mation |
| Ex- | | | MS | | of |
| am- | | | M + | M − | struc- |
| ple | Chemical structure | NMR | H | H | ture |
| F-13 | | (400 MHz, CDCl3) 0.73-0.82 (m, 2H), 1.09-1.22 (m, 4H), 1.27 (dt, J = 18.38, 5.13 Hz, 3H), 1.47-1.53 (m, 2H), 1.65 (dt, J = 30.07, 13.12 Hz, 5H), 2.56 (dd, J = 9.26, 7.28 Hz, 2H), 3.08 (t, J = 6.84 Hz, 2H), 3.32 (t, J = 6.84 Hz, 2H), 3.84 (q, J = 7.28 Hz, 2H), 7.42 (ddd, J = 17.31, 8.60, 5.18 Hz, 3H), 7.65 (d, J = | 405 | 403 | |
| | | 8.16 Hz, 1H), 7.78-7.81 (m, 2H), 7.93- | | | |
| | | 7.99 (m, 1H), 9.97 (s, 1H) | | | |
| F-14 | | (400 MHz, CDCl3) 1.12 (t, J = 7.50 Hz, 3H), 2.40 (d, J = 7.50 Hz, 2H), 3.08 (s, 4H), 3.25 (t, J = 8.38 Hz, 4H), 3.78 (t, J = 8.93 Hz, 1H), 7.22 (d, J = 9.70 Hz, 4H), 7.46 (t, J = 7.94 Hz, 3H), 7.67 (d, J = 8.16 Hz, 1H), 7.85 (s, 2H), 7.96 (d, J = 7.28 Hz, 1H), 8.30 (s, 1H) | 411 | 409 | |
| F-15 | | (400 MHz, CDCl3) 1.39 (t, J = 7.39 Hz, 3H), 2.28 (s, 3H), 3.12 (dd, J = 9.37, 4.74 Hz, 4H), 3.31 (dd, J = 15.11, 8.27 Hz, 2H), 3.49 (dt, J = 12.57, 5.68 Hz, 2H), 3.62-3.71 (m, 1H), 3.98 (q, J = 7.28 Hz, 2H), 7.08 (d, J = 8.60 Hz, 1H), 7.17- 7.20 (m, 2H), 7.24 (dd, J = 5.29, 3.53 Hz, 2H), 7.34 (s, 1H), 8.05 (d, J = 8.38 | 435 | 433 | |
| | | Hz, 1H), 8.10 (s, 1H) | | | |
| F-16 | | (400 MHz, CDCl3) 1.38 (t, J = 7.39 Hz, 3H), 2.24 (s, 6H), 3.07 (dd, J = 9.59, 4.96 Hz, 4H), 3.31 (dd, J = 15.11, 8.49 Hz, 2H), 3.47 (dd, J = 15.44, 9.48 Hz, 2H), 3.61-3.70 (m, 1H), 3.98 (q, J = 7.28 Hz, 2H), 6.69 (s, 1H), 7.16-7.24 (m, 6H), 8.96 (s, 1H) | 389 | 387 | |

TABLE 133

| | | | | | Infor- |
|---|---|---|---|---|---|
| Ex- | | | MS | | mation |
| am- | | | M + | M − | of |
| ple | Chemical structure | NMR | H | H | structure |
| F-17 | | (400 MHz, CDCl3) 0.90-0.99 (m, 2H), 1.06-1.21 (m, 3H), 1.26-1.34 (m, 3H), 1.60-1.80 (m, 6H), 2.56 (dd, J = 12.24, 7.17 Hz, 2H), 3.12 (t, J = 6.51 Hz, 2H), 3.27 (t, J = 6.51 Hz, 2H), 3.83-3.91 (m, 2H), 7.47 (tt, J = 14.34, 6.36 Hz, 3H), 7.65 (d, J = 8.16 Hz, 1H), 7.83 (dd, J = 18.53, 7.50 Hz, 2H), 7.96 (d, J = 7.94 Hz, 1H), 9.47 (s, 1H) | 391 | 389 | |
| F-18 | | (400 MHz, DMSO-D6) 1.26 (t, J = 7.19 Hz, 3H), 2.70 (t, J = 7.20 Hz, 2H), 2.94 (t, J = 7.30 Hz, 2H), 3.12-3.41 (m, 4H), 3.77 (td, J = 16.12, 7.03 Hz, 1H), 4.00 (q, J = 7.27 Hz, 2H), 4.72 (d, J = 5.57 Hz, 2H), 7.16 (dt, J = 9.43, 3.71 Hz, 2H), 7.23-7.26 (m, 2H), 7.41-7.46 (m, 2H), 7.50-7.57 (m, 2H), 7.83 (dd, J = 5.80, 3.71 Hz, 1H), 7.92-7.95 (m, 1H), 8.04 (t, J = 4.41 Hz, 1H), 8.51 (t, J = 5.80 Hz, 1H) | 425 | 423 | |
| F-19 | | (400 MHz, CDCl3) 0.95 (d, J = 6.40 Hz, 6H), 1.33 (t, J = 7.28 Hz, 3H), 1.67 (t, J = 5.95 Hz, 3H), 2.68 (t, J = 7.83 Hz, 2H), 3.13 (t, J = 5.95 Hz, 2H), 3.23 (t, J = 6.18 Hz, 2H), 3.88 (q, J = 7.35 Hz, 2H), 7.48 (tt, J = 17.64, 6.65 Hz, 3H), 7.65 (d, J = 7.94 Hz, 1H), 7.82 (d, J = 8.16 Hz, 1H), 7.94 (t, J = 8.49 Hz, 2H), 9.14 (s, 1H) | 365 | 363 | |

TABLE 134

| | | | | | Infor- |
|---|---|---|---|---|---|
| Ex- | | | MS | | mation |
| am- | | | M + | M − | of |
| ple | Chemical structure | NMR | H | H | structure |
| F-20 | | (400 MHz, CDCl3) 1.32-1.34 (m, 6H), 1.74- 1.77 (m, 3H), 1.87-1.90 (m, 4H), 2.57 (t, J = 12.02 Hz, 1H), 3.12 (t, J = 6.18 Hz, 2H), 3.24 (t, J = 6.18 Hz, 2H), 3.89 (q, J = 7.35 Hz, 2H), 7.47 (ddd, J = 24.92, 13.23, 5.51 Hz, 3H), 7.65 (d, J = 7.94 Hz, 1H), 7.82 (d, J = 8.16 Hz, 1H), 7.94 (dd, J = 13.56, 8.05 Hz, 2H), 9.25 (s, 1H) | 377 | 375 | |
| F-21 | | (400 MHz, CDCl3) 1.12 (t, J = 9.81 Hz, 3H), 1.33 (dt, J = 27.94, 9.92 Hz, 5H), 1.55 (s, 1H), 1.66 (d, J = 13.45 Hz, 2H), 1.81 (d, J = 12.57 Hz, 2H), 2.80 (t, J = 6.95 Hz, 2H), 3.02 (t, J = 6.95 Hz, 2H), 3.32 (dd, J = 15.22, 8.38 Hz, 2H), 3.48 (dd, J = 15.44, 9.48 Hz, 2H), 3.61-3.71 (m, 2H), 3.98 (q, J = 7.28 Hz, 2H), 6.23 (s, 1H), 7.21 (tt, J = 12.68, 4.23 Hz, 4H) | 367 | 365 | |
| F-22 | | (400 MHz, CDCl3) 1.35 (t, J = 7.28 Hz, 3H), 2.93 (t, J = 6.62 Hz, 2H), 3.02 (dd, J = 13.23, 6.62 Hz, 2H), 3.29 (dd, J = 15.11, 8.49 Hz, 2H), 3.43 (dd, J = 15.33, 9.37 Hz, 2H), 3.61 (q, J = 9.04 Hz, 1H), 3.95 (q, J = 7.35 Hz, 2H), 4.40 (d, J = 5.95 Hz, 2H), 7.17-7.24 (m, 9H), 7.43 (s, 1H) | 375 | 373 | |

TABLE 135

| Ex- | | | MS | | Information of |
|---|---|---|---|---|---|
| ample | Chemical structure | NMR | M + H | M − H | structure |
| F-23 | | (400 MHz, CDCl3) 1.39 (t, J = 7.28 Hz, 3H), 3.08 (dd, J = 5.07, 3.31 Hz, 4H), 3.32 (dd, J = 15.22, 8.38 Hz, 2H), 3.47 (dd, J = 15.33, 9.37 Hz, 2H), 3.65 (q, J = 8.97 Hz, 1H), 3.99 (q, J = 7.28 Hz, 2H), 7.04 (t, J = 7.50 Hz, 1H), 7.21 (ddd, J = 16.21, 7.83, 5.07 Hz, 6H), 7.54 | 361 | 359 | |
| | | (d, J = 7.50 Hz, 2H), 9.22 (s, 1H) | | | |
| F-24 | | (400 MHz, CDCl3) 1.36 (t, J = 7.28 Hz, 3H), 1.65 (s, 6H), 1.76 (s, 3H), 1.94 (d, J = 2.87 Hz, 6H), 2.73 (t, J = 6.95 Hz, 2H), 2.99 (t, J = 6.95 Hz, 2H), 3.32 (dd, J = 15.11, 8.27 Hz, 2H), 3.49 (dd, J = 15.22, 9.48 Hz, 2H), 3.65 (q, J = 9.04 Hz, 1H), 3.97 (q, J = 7.35 Hz, 2H), 5.68 (s, 1H), 7.17-7.20 (m, 2H), 7.24 (t, | 419 | 417 | |
| | | J = 4.41 Hz, 2H) | | | |
| F-25 | | (400 MHz, CDCl3) 0.94 (t, J = 13.78 Hz, 9H), 1.33 (t, J = 7.28 Hz, 3H), 1.64-1.68 (m, 2H), 2.61- 2.66 (m, 2H), 3.13 (t, J = 6.40 Hz, 2H), 3.24 (t, J = 6.29 Hz, 2H), 3.88 (q, J = 7.28 Hz, 2H), 7.48 (ddd, J = 26.91, 14.22, 6.29 Hz, 3H), 7.66 (d, J = 8.16 Hz, 1H), 7.82 (d, | 379 | 377 | |
| | | J = 7.94 Hz, 1H), 7.93 (dd, J = 13.23, | | | |
| | | 7.94 Hz, 2H), 9.22 (s, 1H) | | | |

TABLE 136

| Ex- | | | MS | | Information |
|---|---|---|---|---|---|
| ample | Chemical structure | NMR | M + H | M − H | of structure |
| F-26 | | (400 MHz, DMSO-D6) 1.37 (t, J = 7.17 Hz, 3H), 1.56 (dd, J = 8.71, 5.62 Hz, 2H), 2.66 (d, J = 9.26 Hz, 1H), 3.26-3.36 (m, 5H), 3.84 (t, J = 8.49 Hz, 1H), 4.17 (t, J = 4.85 Hz, 2H), 7.16-7.19 (m, 2H), 7.27 (dd, J = 5.29, 3.31 Hz, 2H), 7.48-7.58 (m, 3H), 7.77 (t, J = 6.84 Hz, 2H), 7.95 (dd, J = 6.18, 3.31 Hz, 1H), 8.11 (d, J = 9.26 Hz, 1H), 10.37 (s, 1H) | 423 | 421 | Racemic form |
| F-27 | | (400 MHz, DMSO-D6) 1.34 (t, J = 7.06 Hz, 3H), 1.48 (d, J = 3.53 Hz, 1H), 1.88 (t, J = 10.59 Hz, 1H), 2.56 (dd, J = 14.00, 6.29 Hz, 1H), 3.18- 3.23 (m, 5H), 3.75 (dd, J = 17.09, 8.49 Hz, 1H), 4.10 (d, J = 6.84 Hz, 2H), 7.15 (dd, J = 5.29, 3.31 Hz, 2H), 7.22 (dd, J = 11.03, 4.19 Hz, 2H), 7.47 (tt, J = 18.86, 6.80 Hz, 4H), 7.72 (d, J = 7.06 Hz, 1H), 7.94 (dt, J = 22.50, 4.69 Hz, 2H), 10.20 (s, 1H) | 423 | 421 | Racemic form |
| F-28 | | (400 MHz, CDCl3) 0.84 (t, J = 7.17 Hz, 6H), 1.18-1.38 (m, 11H), 2.83 (dd, J = 13.78, 6.95 Hz, 2H), 3.03 (t, J = 6.84 Hz, 2H), 3.31 (dd, J = 15.22, 8.38 Hz, 2H), 3.48 (dd, J = 15.44, 9.26 Hz, 2H), 3.61- 3.71 (m, 1H), 3.86 (s, 1H), 3.97 (q, J = 7.28 Hz, 2H), 6.00 (s, 1H), 7.17- 7.20 (m, 2H), 7.24 (dd, J = 5.29, | 383 | 381 | |
| | | 3.53 Hz, 2H) | | | |

TABLE 137

| Ex- | | | | | Informa- |
|---|---|---|---|---|---|
| am- | | | MS | | tion of |
| ple | Chemical structure | NMR | M + H | M − H | structure |
| F-29 | | (400 MHz, CDCl3) 1.43 (t, J = 7.28 Hz, 3H), 3.12 (dd, J = 9.04, 4.41 Hz, 4H), 3.35 (dd, J = 15.33, 8.49 Hz, 2H), 3.47 (dd, J = 15.33, 9.37 Hz, 2H), 3.70 (t, J = 9.04 Hz, 1H), 4.03 (q, J = 7.28 Hz, 2H), 6.98 (t, J = 1.87 Hz, 1H), 7.18 (dd, J = 5.18, 3.42 Hz, 2H), 7.23 (t, J = 4.41 Hz, 2H), 7.54 (d, J = 1.76 | 429 | 427 | |
| | | Hz, 2H), 10.15 (s, 1H) | | | |
| F-30 | | (400 MHz, CDCl3) 1.39 (t, J = 7.28 Hz, 3H), 3.10 (t, J = 6.40 Hz, 2H), 3.17 (t, J = 6.06 Hz, 2H), 3.32 (dd, J = 15.11, 8.27 Hz, 2H), 3.46- 3.52 (m, 2H), 3.62-3.71 (m, 1H), 3.98 (q, J = 7.28 Hz, 2H), 7.15-7.25 (m, 6H), 8.21 (d, J = 7.06 Hz, 1H), 8.57 (s, 1H) | 429 | 427 | |
| F-31 | | (400 MHz, CDCl3) 1.39 (t, J = 7.39 Hz, 3H), 3.12 (dd, J = 20.29, 6.40 Hz, 4H), 3.32 (dd, J = 15.11, 8.27 Hz, 2H), 3.48 (dd, J = 15.44, 9.48 Hz, 2H), 3.62-3.71 (m, 1H), 3.98 (q, J = 7.28 Hz, 2H), 7.17- 7.26 (m, 6H), 8.23 (d, J = 8.82 Hz, 1H), 8.52 (s, 1H) | 429 | 427 | |
| F-32 | | (400 MHz, CDCl3) 1.28-1.37 (m, 3H), 1.50 (t, J = 9.81 Hz, 3H), 2.85 (dd, J = 16.10, 3.53 Hz, 1H), 3.09- 3.69 (m, 7H), 3.83-4.02 (m, 2H), 7.21 (dt, J = 19.92, 4.74 Hz, 4H), 7.39-7.44 (m, 3H), 7.64 (d, J = 8.16 Hz, 1H), 7.83 (tt, J = 14.67, 5.77 Hz, 3H), 8.86 (s, 1H) | 425 | 423 | Racemic form |

TABLE 138

| Ex- | | | MS | | Information of |
|---|---|---|---|---|---|
| ample | Chemical structure | NMR | M + H | M − H | structure |
| F-33 | | (400 MHz, DMSO-D6) 1.34 (dd, J = 15.55, 7.39 Hz, 6H), 2.94 (s, 1H), 3.33-3.90 (m, 9H), 7.17 (t, J = 2.65 Hz, 2H), 7.25 (s, 2H), 7.46- 7.58 (m, 4H), 7.75 (d, J = 9.26 Hz, 1H), 7.91 (d, J = 7.28 Hz, 2H), 10.04 (s, 1H) | 425 | 423 | Racemic form |
| F-34 | | (400 MHz, DMSO-D6) 1.37 (t, J = 7.17 Hz, 3H), 3.19-3.42 (m, 6H), 3.87 (t, J = 8.60 Hz, 1H), 4.18 (dd, J = 16.87, 9.37 Hz, 2H), 5.27 (s, 1H), 6.03 (brs, 1H), 7.17-7.20 (m, 2H), 7.27 (s, 2H), 7.46-7.54 (m, 3H), 7.66 (d, J = 7.50 Hz, 1H), 7.76 (d, J = 8.16 Hz, 1H), 7.92 (t, | 427 | 425 | Racemic form |
| | | J = 4.63 Hz, 1H), 8.09 (d, J = 6.84 Hz, | | | |
| | | 1H), 10.11 (s, 1H) | | | |
| F-35 | | (400 MHz, CDCl3) 0.84-0.91 (m, 2H), 1.13-1.27 (m, 7H), 1.50- 1.68 (m, 9H), 1.80-1.99 (m, 5H), 2.20-2.25 (m, 1H), 2.55-2.61 (m, 2H), 2.98-3.04 (m, 1H), 3.17- 3.23 (m, 1H), 3.66-3.75 (m, 1H), 3.87-3.95 (m, 1H), 7.36 (t, J = 7.86 Hz, 1H), 7.42-7.50 (m, 2H), 7.52-7.64 (m, 3H), 7.81- 7.77 (m, 2H) | 459 | 457 | Racemic form |
| F-36 | | (400 MHz, CDCl3) 0.62 (q, J = 12.28 Hz, 2H), 1.03-1.11 (m, 3H), 1.36-1.53 (m, 8H), 1.59 (d, J = 8.60 Hz, 3H), 1.75-1.80 (m, 2H), 2.50 (dt, J = 17.64, 6.56 Hz, 3H), 3.19 (s, 1H), 3.98 (q, J = 7.28 Hz, 2H), 7.45-7.49 (m, 3H), 7.71 (d, J = 8.38 Hz, 1H), 7.86 (d, J = 7.72 Hz, 2H), 8.14 (t, J = 4.63 Hz, 1H), 9.99 (s, 1H) | 417 | 415 | Racemic form |

TABLE 139

| Ex- | | | | | Informa- |
|---|---|---|---|---|---|
| am- | | | MS | | tion of |
| ple | Chemical structure | NMR | M + H | M − H | structure |
| F-37 | | (400 MHz, CDCl3) 0.85-0.95 (m, 2H), 1.08-1.20 (m, 3H), 1.32 (dt, J = 15.07, 5.18 Hz, 3H), 1.67 (dt, J = 30.29, 11.08 Hz, 9H), 2.17 (dt, J = 23.23, 7.77 Hz, 2H), 2.46 (d, J = 7.28 Hz, 1H), 2.66 (ddt, J = 26.10, 13.75, 4.02 Hz, 2H), 3.96 (dtd, J = 40.91, 14.58, 7.24 Hz, 2H), 7.33 (td, J = 13.78, 7.06 Hz, 1H), 7.41- 7.52 (m, 3H), 7.66 (dd, J = 23.38, 7.28 Hz, 2H), 7.78 (d, J = 7.28 Hz, 1H), 8.73 (s, 1H) | 417 | 415 | Racemic form |
| F-38 | | (400 MHz, DMSO-D6) 0.86-0.95 (m, 2H), 1.07-1.38 (m, 7H), 1.53- 1.76 (m, 7H), 2.16-2.25 (m, 6H), 2.63-2.69 (m, 2H), 2.83 (dd, J = 16.43, 9.81 Hz, 2H), 2.92 (t, J = 6.84 Hz, 2H), 3.91 (q, J = 7.20 Hz, 2H), 6.66 (s, 1H), 7.21 (t, J = 19.19 Hz, 2H), 9.90 (d, J = 18.97 Hz, 1H) | 383 | 381 | |
| F-39 | | (400 MHz, CDCl3) 0.92 (dt, J = 27.86, 7.11 Hz, 2H), 1.09-1.34 (m, 7H), 1.67 (ddt, J = 30.21, 13.45, 5.98 Hz, 7H), 2.25 (d, J = 16.76 Hz, 3H), 2.65-2.69 (m, 2H), 3.09 (dt, J = 34.92, 6.18 Hz, 4H), 3.88 (q, J = 7.28 Hz, 2H), 7.08 (t, J = 7.94 Hz, 1H), 7.17 (d, J = 7.72 Hz, 1H), 7.55 (t, J = 9.26 Hz, 1H), 9.15 (s, 1H) | 403 | 401 | |

TABLE 140

| Ex- | | | | | Informa- |
|---|---|---|---|---|---|
| am- | | | MS | | tion of |
| ple | Chemical structure | NMR | M + H | M − H | structure |
| F-40 | | (400 MHz, CDCl3) 0.90-0.99 (m, 2H), 1.10-1.38 (m, 7H), 1.62-1.78 (m, 7H), 2.17 (s, 3H), 2.28 (t, J = 9.15 Hz, 3H), 2.68 (dt, J = 7.87, 3.58 Hz, 2H), 3.07 (ddd, J = 15.82, 10.20, 3.58 Hz, 4H), 3.84-3.92 (m, 2H), 6.98 (d, J = 15.88 Hz, 2H), 7.56 (dd, J = 15.44, 11.03 Hz, 1H), 8.36 (s, 1H) | 383 | 381 | |
| F-41 | | (400 MHz, CDCl3) 0.91- 1.00 (m, 2H), 1.11-1.39 (m, 7H), 1.64-1.83 (m, 7H), 2.29 (d, J = 15.66 Hz, 3H), 2.65 (tt, J = 10.48, 6.10 Hz, 2H), 2.89 (dd, J = 10.48, 3.64 Hz, 2H), 2.97 (ddd, J = 16.82, 8.55, 4.47 Hz, 2H), 3.84-3.92 (m, 2H), 4.38 (dd, J = 12.13, 5.95 Hz, 2H), 6.93 (s, 1H), 6.99- | 383 | 441 (+CH3CO2) | |
| | | 7.08 (m, 3H), 7.19 (ddd, J = | | | |
| | | 16.32, 9.26, 3.42 Hz, 1H) | | | |
| F-42 | | (400 MHz, CDCl3) 0.91-1.00 (m, 2H), 1.10-1.38 (m, 7H), 1.64-1.83 (m, 7H), 2.34 (dd, J = 10.26, 6.51 Hz, 3H), 2.65 (tt, J = 10.48, 6.03 Hz, 2H), 2.89 (dd, J = 10.37, 3.75 Hz, 2H), 2.98 (dt, J = 16.10, 4.02 Hz, 2H), 3.84-3.90 (m, 2H), 4.38 (dd, J = 12.13, 5.73 Hz, 2H), 6.94 (s, 1H), 7.02 (ddd, J = 12.46, | 383 | 441 (+CH3CO2) | |
| | | 6.51, 4.96 Hz, 3H), 7.18 (dt, | | | |
| | | J = 13.89, 5.57 Hz, 1H) | | | |

TABLE 141

| Ex- | | | | | Informa- |
|---|---|---|---|---|---|
| am- | | | MS | | tion of |
| ple | Chemical structure | NMR | M + H | M − H | structure |
| F- 43 | | (400 MHz, CDCl3) 0.88-1.00 (m, 2H), 1.11-1.37 (m, 7H), 1.64-1.78 (m, 7H), 2.32 (d, J = 10.14 Hz, 3H), 2.66 (tt, J = 8.27, 2.72 Hz, 2H),2.79- 2.89 (m, 2H), 2.96-3.00 (m, 2H), 3.83-3.92 (m, 2H), 4.37 (t, J = 8.71 Hz, 2H), 7.03 (d, J = 5.29 Hz, 1H), 7.10 (dt, J = | 383 | 441 (+CH3CO2) | |
| | | 14.26, 4.14 Hz, 4H) | | | |
| F- 44 | | (400 MHz, CDCl3) 0.90- 0.99 (m, 2H), 1.16-1.34 (m, 7H), 1.69 (ddd, J = 35.56, 16.60, 10.53 Hz, 7H), 2.60-2.66 (m, 2H), 2.86 (t, J = 6.62 Hz, 2H), 2.97 (dd, J = 8.27, 4.52 Hz, 2H), 3.82 (q, J = 7.35 Hz, 2H), | 419 | 477 (+CH3CO2) | |
| | | 4.86 (d, J = 5.51 Hz, 2H), 6.77 | | | |
| | | (s, 1H), 7.39 (q, J = 6.62 Hz, | | | |
| | | 2H), 7.46-7.53 (m, | | | |
| | | 2H), 7.76-7.80 (m, 1H), | | | |
| | | 7.85 (dd, J = 7.17, 2.10 Hz, | | | |
| | | 1H), 7.97 (t, J = 4.52 Hz, 1H) | | | |
| F- 45 | | (400 MHz, CDCl3) 0.96 (q, J = 11.25 Hz, 2H), 1.24 (dtt, J = 52.05, 17.31, 5.84 Hz, 7H), 1.59-1.79 (m, 7H), 2.67 (dt, J = 7.94, 3.53 Hz, 2H), 2.89 (t, J = 6.62 Hz, 2H), 2.96-3.02 (m, 2H), 3.86 (q, J = 7.35 Hz, 2H), 4.40 (d, J = 5.95 Hz, 2H), 6.77 (s, 1H), 7.20-7.32 (m, 5H) | 369 | 427 (+CH3CO2) | |
| F- 46 | | (400 MHz, CDCl3) 0.82-1.03 (m, 9H), 1.28-1.39 (m, 3H), 1.66 (ddt, J = 25.33, 18.64, 8.10 Hz, 2H), 2.16- 2.38 (m, 6H), 2.64 (tt, J = 6.73, 3.90 Hz, 2H), 2.89-3.19 (m, 4H), 3.91 (ddd, J = 31.26, 14.83, 9.65 Hz, 2H), 6.71 (d, J = 11.47 Hz, 1H), 7.17 (t, | 357 | 355 | |
| | | J = 9.04 Hz, 2H), 9.42 (s, 1H) | | | |

TABLE 142

| Ex- | | | | | Informa- |
|---|---|---|---|---|---|
| am- | | | MS | | tion of |
| ple | Chemical structure | NMR | M + H | M − H | structure |
| F-47 | | (400 MHz, CDCl3) 0.96 (dq, J = 26.96, 5.37 Hz, 9H), 1.30 (q, J = 11.10 Hz, 3H), 1.63-1.68 (m, 2H), 2.10-2.22 (m, 3H), 2.30 (t, J = 15.33 Hz, 3H), 2.66 (dt, J = 16.10, 7.00 Hz, 2H), 3.07 (ddd, J = 16.49, 10.20, 3.58 Hz, 4H), 3.88 (q, J = 7.35 Hz, 2H), 6.96 (s, 2H), 7.58 (t, J = 4.41 Hz, 1H), 8.40 (s, 1H) | 357 | 355 | |
| F-48 | | (400 MHz, CDCl3) 1.11 (s, 2H), 1.33 (dd, J = 17.09, 9.81 Hz, 3H), 1.56 (dd, J = 22.72, 13.89 Hz, 4H), 1.76 (dd, J = 15.77, 7.83 Hz, 5H), 2.17 (s, 3H), 2.26 (s, 3H), 2.67 (t, J = 7.94 Hz, 2H), 3.09 (dd, J = 15.55, 6.06 Hz, 4H), 3.90 (q, J = 7.35 Hz, 2H), 6.94 (d, J = 7.28 Hz, 2H), 7.49 (d, J = 8.38 Hz, 1H), 8.79 (s, 1H) | 369 | 367 | |
| F-49 | | (400 MHz, CDCl3) 1.12 (s, 2H), 1.34 (t, J = 7.28 Hz, 3H), 1.56 (d, J = 36.61 Hz, 4H), 1.79 (t, J = 7.72 Hz, 5H), 2.22 (d, J = 18.31 Hz, 6H), 2.69 (t, J = 7.83 Hz, 2H), 3.08 (s, 4H), 3.93 (q, J = 7.35 Hz, 2H), 6.69 (s, 1H), 7.20 (d, J = 17.86 Hz, 2H), 9.53 (s, 1H), | 369 | 367 | |
| F-50 | | (400 MHz, CDCl3) 0.96 (d, J = 6.45 Hz, 6H), 1.32 (t, J = 7.66 Hz, 3H), 1.65-1.71 (m, 3H), 2.27 (s, 6H), 2.66-2.70 (m, 2H), 2.99-3.07 (m, 4H), 3.88 (q, J = 7.39 Hz, 2H), 6.71 (s, 1H), 7.16 (s, 2H), 8.71 (s, 1H) | 343 | 341 | |

TABLE 143

| Ex- | | | | | Informa- |
|---|---|---|---|---|---|
| am- | | | MS | | tion of |
| ple | Chemical structure | NMR | M + H | M − H | structure |
| F-51 | | (400 MHz, CDCl3) 0.96 (d, J = 6.45 Hz, 6H), 1.32 (t, J = 7.45 Hz, 3H), 1.64-1.69 (m, 3H), 2.18 (s, 3H), 2.26 (s, 3H), 2.66-2.70 (m, 2H), 3.05-3.08 (m, 4H), 3.88 (q, J = 7.39 Hz, 2H), 6.96-6.98 (m, 2H), 7.61 (d, J = 8.87 Hz, 1H), 8.20 (s, 1H) | 343 | 341 | |
| F-52 | | (400 MHz, CDCl3) 0.95 (t, J = 5.64 Hz, 6H), 1.33 (t, J = 8.87 Hz, 3H), 1.64-1.69 (m, 3H), 2.28 (s, 3H), 2.66-2.70 (m, 2H), 3.04- 3.12 (m, 4H), 3.88 (q, J = 7.25 Hz, 2H), 7.08 (dd, J = 8.46, 8.06 Hz, 1H), 7.16 (d, J = 8.06 Hz, 1H), 7.63 (d, J = 7.66 Hz, 1H), 8.79 (s, 1H) | 363 | 361 | |
| F-53 | | (400 MHz, CDCl3) 0.96 (s, 9H), 1.34 (t, J = 7.28 Hz, 3H), 1.63-1.68 (m, 2H), 2.28 (s, 3H), 2.62-2.66 (m, 2H), 3.09 (dt, J = 27.49, 6.06 Hz, 4H), 3.88 (q, J = 7.35 Hz, 2H), 7.08 (t, J = 7.94 Hz, 1H), 7.17 (d, J = 8.16 Hz, 1H), 7.60 (d, J = 7.94 Hz, 1H), 8.98 (s, 1H) | 377 | 375 | |
| F-54 | | (400 MHz, CDCl3) 1.12 (s, 2H), 1.33 (t, J = 7.28 Hz, 3H), 1.57 (dd, J = 22.94, 13.89 Hz, 4H), 1.79 (dd, J = 22.27, 14.34 Hz, 5H), 2.27 (s, 3H), 2.68 (t, J = 7.94 Hz, 2H), 3.09 (dd, J = 24.92, 6.62 Hz, 4H), 3.88 (q, J = 7.35 Hz, 2H), 7.08 (t, J = 8.05 Hz, 1H), 7.16 (d, J = 7.72 Hz, 1H), 7.58 (d, J = 7.94 Hz, 1H), 9.04 (s, 1H) | 389 | 387 | |

TABLE 144

| Ex- | | | | | Information |
|---|---|---|---|---|---|
| am- | | | MS | | of |
| ple | Chemical structure | NMR | M + H | M − H | structure |
| F-55 | | (400 MHz, CDCl3) 0.93-0.99 (m, 8H), 1.14-1.22 (m, 2H), 1.61- 1.70 (m, 3H), 2.29 (s, 3H), 2.79 (t, J = 7.72 Hz, 2H), 2.94-3.00 (m, 1H), 3.08 (t, J = 6.06 Hz, 2H), 3.17 (t, J = 6.06 Hz, 2H), 7.08 (t, J = 7.94 Hz, 1H), 7.17 (t, J = 8.16 Hz, 1H), 7.63 (d, J = 7.50 Hz, 1H), 8.85 (s, 1H) | 375 | 373 | |
| F-56 | | (400 MHz, CDCl3) 0.93-1.00 (m, 8H), 1.21 (q, J = 6.57 Hz, 2H), 1.61-1.70 (m, 3H), 2.80 (t, J = 7.77 Hz, 2H), 2.98 (td, J = 7.13, 3.48 Hz, 1H), 3.04 (t, J = 6.49 Hz, 2H), 3.18 (t, J = 6.49 Hz, 2H), 7.06 (t, J = 7.30 Hz, 1H), 7.28 (t, J = 7.28 Hz, 2H), 7.54 (d, J = 8.35 Hz, 2H), 9.17 (s, 1H) | 327 | 325 | |
| F-57 | | (400 MHz, CDCl3) 0.92-1.00 (m, 8H), 1.16-1.21 (m, 2H), 1.65- 1.75 (m, 3H), 2.76-2.80 (m, 2H), 2.79 (dt, J = 11.98, 4.58 Hz, 2H), 2.88 (t, J = 6.84 Hz, 1H), 3.12 (t, J = 6.84 Hz, 2H), 4.41 (d, J = 6.03 Hz, 2H), 6.83 (brs, 1H), 7.20-7.34 (m, 5H) | 341 | 339 | |
| F-58 | | (400 MHz, CDCl3) 0.93-0.99 (m, 8H), 1.16-1.21 (m, 2H), 1.62- 1.72 (m, 3H), 2.77-2.81 (m, 6H), 2.93-2.99 (m, 1H), 3.06 (t, J = 6.96 Hz, 2H), 3.48 (q, J = 6.72 Hz, 2H), 6.43 (brs, 1H), 7.17-7.30 (m, 5H) | 355 | 353 | |

TABLE 145

| Ex- | | | | | Informa- |
|---|---|---|---|---|---|
| am- | | | MS | | tion of |
| ple | Chemical structure | NMR | M + H | M − H | structure |
| F-59 | | (400 MHz, CDCl3) 0.92-0.98 (m, 8H), 1.15-1.20 (m, 2H), 1.65- 1.70 (m, 3H), 2.28 (s, 3H), 2.75- 2.79 (m, 2H), 2.86 (t, J = 6.84 Hz, 2H), 2.92-2.98 (m, 1H), 3.11 (t, J = 6.84 Hz, 2H), 4.39 (d, J = 5.51 Hz, 2H), 6.56 (brs, 1H), 7.11-7.19 (m, 4H) | 355 | 353 | |
| F-60 | | (400 MHz, CDCl3) 0.92-0.97 (m, 8H), 1.15-1.20 (m, 2H), 1.64- 1.71 (m, 3H), 2.32 (s, 3H), 2.76- 2.80 (m, 2H), 2.87 (t, J = 6.95 Hz, 2H), 2.93-2.98 (m, 1H), 3.12 (t, J = 6.84 Hz, 2H), 4.36 (d, J = 5.95 Hz, 2H), 6.66 (brs, 1H), 7.00-7.06 (m, 3H), 7.18 (t, J = 7.60, 1H) | 355 | 353 | |
| F-61 | | (400 MHz, CDCl3) 0.92-1.00 (m, 8H), 1.18 (td, J = 6.96, 5.95 Hz, 2H), 1.67-1.72 (m, 3H), 2.32 (s, 3H), 2.78 (t, J = 7.68 Hz, 2H), 2.86 (t, J = 6.84 Hz, 2H), 2.93-2.98 (m, 1H), 3.12 (t, J = 6.84 Hz, 2H), 4.36 (d, J = 5.80 Hz, 2H), 6.69 (brs, 1H), 7.11 (s, 4H) | 355 | 353 | |
| F-62 | | (400 MHz, CDCl3) 0.89 (dt, J = 8.01, 3.47 Hz, 2H), 0.97 (d, J = 6.18 Hz, 6H), 1.14 (td, J = 8.44, 4.19 Hz, 2H), 1.42 (d, J = 7.06 Hz, 3H), 1.68 (dt, J = 13.75, 5.68 Hz, 3H), 2.76-2.85 (m, 4H), 2.88- 2.94 (m, 1H), 3.06-3.11 (m, 2H), 4.99-5.07 (m, 1H), 6.65 (d, J = 7.06 Hz, 1H), 7.19-7.30 (m, 5H) | 355 | 353 | |

TABLE 146

| Ex- | | | | | Informa- |
|---|---|---|---|---|---|
| am- | | | MS | | tion of |
| ple | Chemical structure | NMR | M + H | M − H | structure |
| F-63 | | (400 MHz, CDCl3) 0.90 (dt, J = 7.88, 3.48 Hz, 2H), 0.98 (d, J = 6.26 Hz, 6H), 1.15 (td, J = 8.41, 4.17 Hz, 2H), 1.43 (d, J = 6.96 Hz, 3H), 1.65-1.75 (m, 3H), 2.77- 2.86 (m, 4H), 2.89-2.95 (m, 1H), 3.07-3.12 (m, 2H), 5.00-5.07 (m, 1H), 6.68 (d, J = 7.88 Hz, 1H), 7.20- 7.31 (m, 5H) | 355 | 353 | |
| F-64 | | (400 MHz, CDCl3) 0.95-0.99 (m, 8H), 1.16-1.22 (m, 2H), 1.62- 1.70 (m, 3H), 2.25 (s, 3H), 2.79 (t, J = 7.83 Hz, 2H), 2.94-3.00 (m, 1H), 3.08 (dd, J = 11.58, 4.96 Hz, 2H), 3.17 (dd, J = 11.36, 4.74 Hz, 2H), 7.02 (t, J = 7.61 Hz, 1H), 7.13- 7.18 (m, 2H), 7.83 (d, J = 7.94 Hz, 1H), 8.44 (s, 1H) | 341 | 339 | |
| F-65 | | (400 MHz, CDCl3) 0.95-0.99 (m, 8H), 1.20 (q, J = 6.65 Hz, 2H), 1.61- 1.72 (m, 3H), 2.32 (s, 3H), 2.80 (t, J = 7.88 Hz, 2H), 2.95-3.04 (m, 3H), 3.17 (t, J = 6.61 Hz, 2H), 6.88 (d, J = 7.42 Hz, 1H), 7.16 (t, J = 7.65 Hz, 1H), 7.34 (d, J = 8.35 Hz, 1H), 7.37 (s, 1H), 8.96 (s, 1H) | 341 | 339 | |
| F-66 | | (400 MHz, CDCl3) 0.95-0.99 (m, 8H), 1.20 (q, J = 6.65 Hz, 2H), 1.63- 1.71 (m, 3H), 2.29 (s, 3H), 2.80 (t, J = 7.77 Hz, 2H), 2.95-3.03 (m, 3H), 3.17 (t, J = 6.61 Hz, 2H), 7.08 (d, J = 8.12 Hz, 2H), 7.41 (d, J = 8.35 Hz, 2H), 8.95 (s, 1H) | 341 | 339 | |

TABLE 147

| | | | | | Infor- |
|---|---|---|---|---|---|
| | | | | | ma- |
| Ex- | | | | | tion of |
| am- | | | MS | | struc- |
| ple | Chemical structure | NMR | M + H | M − H | ture |
| F- 67 | | (400 MHz, CDCl3) 0.90-0.98 (m, 8H), 1.15-1.20 (m, 2H), 1.66-1.72 (m, 3H), 2.31 (s, 3H), 2.71-12.81 (m, 6H), 2.92-2.98 (m, 1H), 3.06 (t, J = 6.95 Hz, 2H), 3.45 (dd, J = 13.12, 7.17 Hz, 2H), 6.31 (brs, 1H), 7.05 (d, J = 8.40 Hz, 2H), 7.09 (d, J = 7.94 Hz, 2H) | 369 | 367 | |
| F- 68 | | (400 MHz, CDCl3) 0.94- 0.99 (m, 8H), 1.16-1.21 (m, 2H), 1.67-1.72 (m, 3H), 2.33 (s, 3H), 2.73-2.81 (m, 6H), 2.94-2.99 (m, 1H), 3.08 (t, J = 6.96 Hz, 2H), 3.47 (dd, J = 13.10, 7.07 Hz, 2H), 6.33 (brs, 1H), 6.97 (d, J = 7.88 Hz, 1H), 7.01 (s, 1H), 7.03 (d, J = 8.12 Hz, 1H), 7.18 (t, J = 7.54 Hz, 1H) | 369 | 367 | |
| F- 69 | | (400 MHz, CDCl3) 0.98 (t, J = 6.84 Hz, 11H), 1.18 (d, J = 5.73 Hz, 2H), 1.63-1.67 (m, 2H), 2.19 (s, 3H), 2.26 (s, 3H), 2.74-2.79 (m, 2H), 2.96-2.99 (m, 1H), 3.05 (t, J = 6.40 Hz, 2H), 3.17 (t, J = 6.40 Hz, 2H), 6.96 (s, 2H), 7.60 (d, J = 8.60 Hz, 1H), 8.35 (s, 1H) | 369 | 367 | |
| F- 70 | | (400 MHz, CDCl3) 0.95-1.00 (m, 8H), 1.18-1.23 (m, 2H), 1.65-1.70 (m, 3H), 2.12 (s, 6H), 2.79-2.82 (m, 2H), 2.97-3.02 (m, 1H), 3.17-3.12 (m, 2H), 3.20-3.23 (m, 2H), 7.01-7.09 (m, 3H), 7.98 (brs, 1H) | 355 | 353 | |

TABLE 148

| Ex- | | | | | Informa- |
|---|---|---|---|---|---|
| am- | | | MS | | tion of |
| ple | Chemical structure | NMR | M + H | M − H | structure |
| F-71 | | (400 MHz, CDCl3) 0.95-0.98 (m, 8H), 1.16-1.21 (m, 2H), 1.63- 1.70 (m, 3H), 2.19 (s, 3H), 2.29 (s, 3H), 2.77-2.81 (m, 2H), 2.94- 3.00 (m, 1H), 3.06 (t, J = 6.29 Hz, 2H), 3.17 (t, J = 6.40 Hz, 2H), 6.84 (d, J = 7.72 Hz, 1H), 7.02 (d, J = 7.72 Hz, 1H), 7.65 (s, 1H), 8.30 (brs, 1H) | 355 | 353 | |
| F-72 | | (400 MHz, CDCl3) 0.96-0.99 (m, 8H), 1.15-1.23 (m, 5H), 1.67- 1.71 (m, 3H), 2.58 (q, J = 7.50 Hz, 2H), 2.79-2.83 (m, 2H), 2.95- 3.01 (m, 1H), 3.09 (t, J = 6.00 Hz, 2H), 3.19 (t, J = 6.40 Hz, 2H), 7.09 (t, J = 6.96 Hz, 1H), 7.16-7.19 (m, 2H), 7.76 (d, J = 7.88 Hz, 1H), 8.39 (brs, 1H) | 355 | 353 | |
| F-73 | | (400 MHz, CDCl3) 0.94-1.00 (m, 8H), 1.17-1.22 (m, 2H), 1.64- 1.70 (m, 3H), 2.79 (dd, J = 9.48, 6.40 Hz, 2H), 2.96-3.02 (m, 1H), 3.06 (t, J = 6.95 Hz, 2H), 3.18 (t, J = 6.84 Hz, 2H), 3.88 (s, 3H), 6.86 (dd, J = 8.05, 1.21 Hz, 1H), 6.93 (td, J = 7.83, 1.32 Hz, 1H), 7.02 (td, J = 7.77, 1.62 Hz, 1H), 8.25 (brs, 1H), 8.31 (dd, J = 7.94, 1.32 Hz, 1H) | 357 | 355 | |
| F-74 | | (400 MHz, CDCl3) 0.94-0.98 (m, 8H), 1.16-1.21 (m, 2H), 1.62- 1.71 (m, 3H), 2.19 (s, 3H), 2.21 (s, 3H), 2.79 (t, J = 7.94 Hz, 2H), 2.94- 3.01 (m, 3H), 3.16 (t, J = 6.62 Hz, 2H), 7.02 (d, J = 7.94 Hz, 1H), 7.24- 7.29 (m, 2H), 8.73 (brs, 1H) | 355 | 353 | |

TABLE 149

| Ex- | | | | | Informa- |
|---|---|---|---|---|---|
| am- | | | MS | | tion of |
| ple | Chemical structure | NMR | M + H | M − H | structure |
| F-75 | | (400 MHz, CDCl3) 0.95-0.99 (m, 8H), 1.16-1.23 (m, 8H), 1.63- 1.70 (m, 3H), 2.78-2.82 (m, 2H), 2.95-3.03 (m, 2H), 3.08 (t, J = 6.29 Hz, 2H), 3.18 (t, J = 6.29 Hz, 2H), 7.14-7.16 (m, 2H), 7.24- 7.26 (m, 1H), 7.58-7.60 (m, 1H), 8.27 (brs, 1H) | 369 | 367 | |
| F-76 | | (400 MHz, CDCl3) 0.94-0.98 (m, 8H), 1.16-1.22 (m, 2H), 1.62- 1.71 (m, 3H), 2.27 (s, 6H), 2.78- 2.81 (m, 2H), 2.94-3.01 (m, 3H), 3.14-3.17 (m ,2H), 6.70 (s, 1H), 7.16 (s, 2H), 8.77 (brs, 1H) | 355 | 353 | |
| F-77 | | (400 MHz, CDCl3) 0.92 (t, J = 7.39 Hz, 3H), 0.95-0.99 (m, 8H), 1.17-1.22 (m, 2H), 1.57 (td, J = 14.94, 7.50 Hz, 2H), 1.63-1.68 (m, 3H), 2.53 (t, J = 7.61 Hz, 2H), 2.78-2.82 (m, 2H), 2.95-3.01 (m, 1H), 3.07 (t, J = 6.40 Hz, 2H), 3.18 (t, J = 6.29 Hz, 2H), 7.07 (t, J = 7.39 Hz, 1H), 7.14-7.19 (m, 2H), 7.73 (d, J = 7.94 Hz, 1H), 8.33 (brs, 1H) | 369 | 367 | |
| F-78 | | (400 MHz, CDCl3) 0.99-0.96 (m, 8H), 1.17-1.22 (m, 2H), 1.64- 1.71 (m, 3H), 2.13 (s, 3H), 2.27 (s, 3H), 2.78-2.82 (m, 2H), 2.95- 3.01 (m, 1H), 3.07 (t, J = 5.33 Hz, 2H), 3.19 (t, J = 6.49 Hz, 2H), 6.97 (d, J = 9.51 Hz, 1H), 7.06 (dd, J = 8.12, 7.88 Hz, 1H), 7.49 (d, J = 10.20 Hz, 1H), 8.33 (s, 1H | 355 | 353 | |

TABLE 150

| | | | MS | | Information |
|---|---|---|---|---|---|
| Example | Chemical structure | NMR | M + H | M − H | of structure |
| F-79 | | (400 MHz, CDCl3) 0.94-0.97 (m, 8H), 1.16-1.21 (m, 2H), 1.64- 1.67 (m, 3H), 2.19 (s, 3H), 2.26 (s, 3H), 2.77-2.81 (m, 2H), 2.94- 2.99 (m, 1H), 3.05 (t, J = 6.85 Hz, 2H), 3.17 (t, J = 6.45 Hz, 2H), 6.95- 6.97 (m, 2H), 7.62 (d, J = 8.87 Hz, 1H), 8.28 (s, 1H) | 355 | 353 | |
| F-80 | | (400 MHz, CDCl3) 0.95-0.99 (m, 8H), 1.18-1.23 (m, 2H), 1.65- 1.70 (m, 3H), 2.32 (s, 3H), 2.83- 2.79 (m, 2H), 2.94-3.00 (m, 1H), 3.08-3.17 (m, 4H), 8.18 (d, J = 5.64 Hz, 1H), 8.31 (s, 1H), 8.34 (d, J = 5.64 Hz, 1H), 9.53 (s, 1H) | 342 | 340 | |
| F-81 | | (400 MHz, CDCl3) 0.92-0.98 (m, 8H), 1.18 (dd, J = 12.68, 6.73 Hz, 2H), 1.60-1.69 (m, 3H), 2.76- 2.80 (m, 2H), 2.94-3.00 (m, 1H), 3.23 (s, 4H), 7.41-7.48 (m, 2H), 7.52 (t, J = 7.17 Hz, 1H), 7.65 (d, J = 8.16 Hz, 1H), 7.82 (d, J = 8.16 Hz, 1H), 7.93 (d, J = 7.50 Hz, 1H), 7.99 (d, J = 8.38 Hz, 1H), 9.30 (s, 1H) | 377 | 375 | |
| F-82 | | (400 MHz, CDCl3) 0.95-0.99 (m, 8H), 1.19 (dd, J = 12.79, 7.06 Hz, 2H), 1.63-1.70 (m, 3H), 2.18 (s, 3H), 2.77-2.81 (m, 2H), 2.94- 3.00 (m, 1H), 3.04 (t, J = 6.40 Hz, 2H), 3.17 (t, J = 6.40 Hz, 2H), 3.76 (s, 2H), 6.68-6.70 (m, 2H), 7.51- 7.54 (m, 1H), 8.20 (brs, 1H) | 371 | 369 | |

TABLE 151

| | | | Ms | | Information |
|---|---|---|---|---|---|
| Example | Chemical structure | NMR | M + H | M − H | of structure |
| F-83 | | (400 MHz, CDCl3) 0.96-1.00 (m, 8H), 1.21 (q, J = 6.65 Hz, 2H), 1.67- 1.71 (m, 3H), 1.72-1.82 (m, 1H), 2.49-2.58 (m, 1H), 2.77-2.89 (m, 5H), 2.93-3.02 (m, 2H), 3.14 (t J = 6.96 Hz, 2H), 5.43 (q, J = 7.73 Hz, 1H), 6.45 (d, J = 7.88 Hz, 1H), 7.16- 7.23 (m, 4H) | 367 | 365 | Racemic form |
| F-84 | | (400 MHz, CDCl3) 0.95-0.98 (m, 8H), 1.17-1.22 (m, 2H), 1.63- 1.70 (m, 3H), 2.34 (s 3H), 2.77- 2.81 (m, 2H), 2.94-3.00 (m, 1H), 3.08 (t, J = 5.95 Hz, 3H), 3.17 (d, J = 5.20 Hz, 3H), 7.01 (t, J = 8.16 Hz, 1H), 7.34 (d, J = 8.16 Hz, 1H), 7.68 (d, J = 8.16 Hz, 1H), 8.84 (brs, 1H) | 419 | 417 | |
| F-85 | | (400 MHz, CDCl3) 0.95-0.98 (m, 8H), 1.16-1.21 (m, 2H), 1.61- 1.70 (m, 3H), 2.09 (s, 3H), 2.77- 2.81 (m, 2H), 2.94-2.99 (m, 1H), 3.06 (t, J = 6.40 Hz, 2H), 3.17 (t, J = 6.29 Hz, 2H), 3.80 (s, 3H), 6.66 (d, J = 7.94 Hz, 1H), 7.11 (t, J = 8.16 Hz, 1H), 7.38 (d, J = 8.16 Hz, 1H), 8.29 (brs, 1H) | 371 | 369 | |
| F-86 | | (400 MHz, DMSO-D6) 0.92 (d, J = 6.26 Hz, 6H), 0.95-0.99 (m, 2H), 1.05-1.10 (m, 2H), 1.55- 1.63 (m, 3H), 2.03 (s, 3H), 2.70- 2.74 (m, 2H), 2.81 (t, J = 7.54 Hz, 2H), 3.01 (t, J = 7.54 Hz, 2H), 3.09- 3.14 (m, 1H), 3.72 (s, 3H), 6.78 (d, J = 7.65 Hz, 1H), 6.84 (d, J = 8.12 Hz, 1H), 7.10 (t, J = 8.00 Hz, 1H), 9.18 (s, 1H) | 371 | 369 | |

TABLE 152

| | | | MS | | Information |
|---|---|---|---|---|---|
| Example | Chemical structure | NMR | M + H | M − H | of structure |
| F-87 | | (400 MHz, CDCl3) 0.95-0.99 (m, 8H), 1.17-1.22 (m, 2H), 1.63- 1.70 (m, 3H), 2.20 (s, 3H) 2.78- 2.82 (m, 2H), 2.94-3.00 (m, 1H), 3.08 (t, J = 5.64 Hz, 2H) 3.18 (t, J = 6.45 Hz, 2H), 4.69 (d, J = 4.84 Hz, 2H), 7.18-7.16 (m, 2H), 7.63 (t, J = 4.63 Hz, 1H), 8.47 (s, 1H) | 371 | 369 | |
| F-88 | | (400 MHz, CDCl3) 0.95-0.98 (m, 8H), 1.16-1.22 (m, 2H), 1.64- 1.70 (m, 3H), 2.25 (s, 3H), 2.78- 2.82 (m, 2H), 2.94-3.00 (m, 1H), 3.06 (t, J = 6.04 Hz, 2H), 3.17 (t, J = 6.25 Hz, 2H), 4.61 (d, J = 4.43Hz, 2H), 7.16-7.14 (m, 2H), 7.81 (d, J = 8.87 Hz, 1H), 8.51 (s, 1H) | 371 | 369 | |
| F-89 | | (400 MHz, CDCl3) 0.95-0.98 (m, 8H), 1.16-1.21 (m, 2H), 1.64- 1.71 (m, 3H), 2.22 (s, 3H), 2.78- 2.82 (m, 2H), 2.97-3.02 (m, 1H), 3.11-3.21 (m, 4H), 7.05 (dd, J = 7.66, 4.84 Hz, 1H), 7.51 (dd, J = 7.45, 1.01 Hz, 1H), 8.26 (dd, J = 4.63, 1.41 Hz, 1H), 8.60 (s, 1H) | 342 | 340 | |
| F-90 | | (400 MHz, CDCl3) 0.95-0.98 (m, 8H), 1.18-1.23 (m, 2H),1.64- 1.68 (m, 3H), 2.28 (s, 3H), 2.78- 2.82 (m, 2H), 2.95-3.01 (m, 1H), 3.08-3.11 (m, 2H), 3.16-3.19 (m, 2H), 7.08 (d, J = 4.84 Hz, 1H), 8.24 (d, J = 4.84 Hz 1H), 8.97 (s, 1H), 9.08 (s, 1H) | 342 | 340 | |

TABLE 153

| | | | MS | | Information |
|---|---|---|---|---|---|
| Example | Chemical structure | NMR | M + H | M − H | of structure |
| F-91 | | (400 MHz, CDCl3) 0.95-0.98 (m, 8H), 1.18-1.23 (m, 2H), 1.63- 1.68 (m, 3H), 2.53 (s, 3H), 2.79- 2.82 (m, 2H), 2.95-3.01 (m, 1H), 3.09-3.18 (m, 4H), 7.10 (dd, J = 7.25, 5.64 Hz, 1H), 8.25-8.23 (m, 2H), 9.17 (s, 1H) | 342 | 340 | |
| F-92 | | (400 MHz, CDCl3) 0.95-0.99 (m, 8H), 1.20 (dd, J = 12.64, 7.07 Hz, 2H), 1.65-1.70 (m, 3H), 2.20 (s, 3H), 2.78-2.82 (m, 2H), 2.95- 3.00 (m, 1H), 3.08 (t, J = 6.03 Hz, 2H), 3.18 (t, J = 6.03 Hz, 2H), 3.78 (s, 3H), 6.59 (dd, J = 8.46, 2.43 Hz, 1H), 7.03 (d, J = 8.35 Hz, 1H), 7.64 (d, J = 2.55 Hz, 1H), 8.47 (s, 1H) | 371 | 369 | |
| F-93 | | (400 MHz, CDCl3) 0.96-1.01 (m, 8H), 1.19-1.24 (m,2H), 1.62- 1.72 (m, 3H), 2.79-2.83 (m, 2H), 2.98-3.03 (m, 1H), 3.11-3.21 (m, 4H), 7.03 (t, J = 8.80 Hz, 1H), 7.24 (t, J = 8.00 Hz, 1H), 7.36 (dd, J = 8.12, 1.39 Hz, 1H), 8.29 (d, J = 8.35 Hz, 1H), 8.42 (s, 1H) | 361 | 359 | |
| F-94 | | (400 MHz, CDCl3) 0.89-0.98 (m, 4H), 1.13-1.27 (m, 6H), 1.59- 1.74 (m, 7H), 2.24 (d, J = 22.94 Hz, 3H), 2.77 (dt, J = 8.01, 3.58 Hz, 2H), 2.94-2.99 (m, 1H), 3.14 (dd, J = 10.48, 4.74 Hz 4H),7.07 (t, J = 7.94 Hz, 1H), 7.16 (d, J = 7.50 Hz, 1H), 7.56 (d, J = 7.94 Hz, 1H), 9.24 (s, 1H) | 415 | 413 | |

TABLE 154

| | | | MS | | Information |
|---|---|---|---|---|---|
| Example | Chemical structure | NMR | M + H | M − H | of structure |
| F-95 | | (400 MHz, CDCl3) 0.94 (dt, J = 10.44, 5.62 Hz, 4H), 1.13-1.27 (m, 6H), 1.61-1.76 (m, 7H), 2.18 (s, 3H), 2.26 (s, 3H), 2.78 (t, J = 8.16 Hz, 2H), 2.94-2.98 (m, 1H), 3.06 (t, J = 6.18 Hz, 2H), 3.16 (t, J = 6.06 Hz, 2H), 6.96 (s, 2H), 7.57 (d, J = 8.82 Hz, 1H), 8.51 (s, 1H) | 395 | 393 | |
| F-96 | | (400 MHz, CDCl3) 1.12 (ddd, J = 23.82, 11.80, 6.51 Hz, 4H), 2.15 (s, 3H), 2.25 (s, 3H), 2.99 (t, J = 6.73 Hz, 2H), 3.05-3.09 (m, 1H), 3.15 (t, J = 6.73 Hz, 2H), 4.78 (s, 2H), 6.94 (s, 2H), 7.51 (d, J = 7.28 Hz, 1H), 8.29 (s, 1H) | 315 | 313 | |
| F-97 | | (400 MHz, CDCl3) 1.12 (dd, J = 6.51, 4.96 Hz, 4H), 1.24 (d, J = 20.95 Hz, 9H), 2.19 (s, 3H), 2.26 (s, 3H) 3.07 (dd, J = 12.13, 5.29 Hz, 3H), 3.19 (t, J = 6.51 Hz, 2H), 4.61 (d, J = 17.20 Hz, 2H), 6.95 (s, 2H), 7.60 (d, J = 8.82 Hz, 1H), 8.37 (s. 1H) | 371 | 369 | |
| F-98 | | (400 MHz, CDCl3) 0.87 (d, J = 6.40 Hz, 6H), 0.97 (d, J = 3.97 Hz, 2H), 1.20 (dd, J = 6.95, 2.98 Hz, 2H), 1.27 (d, J = 6.84 Hz, 3H), 1.51 (dq, J = 28.78, 6.65 Hz, 2H), 1.69 (dd, J = 13.12, 7.17 Hz, 1H) 2.24 (s, 3H), 2.93 (td, J = 10.53, 8.16 Hz, 1H), 3.16 (dt, J = 15.73, 6.45 Hz, 5H), 7.07 (t, J = 7.94 Hz, 1H), 7.16 (d, J = 7.72 Hz, 1H), 7.53 (d, J = 7.94 Hz, 1H), 9.28 (s, 1H) | 389 | 387 | Racemic form |

TABLE 155

| | | | MS | | Information |
|---|---|---|---|---|---|
| Example | Chemical structure | NMR | M + H | M − H | of structure |
| F-99 | | (400 MHz, CDCl3) 0.95-1.00 (m, 8H), 1.21 (dd, J = 12.87, 7.07 Hz, 2H), 1.64-1.70 (m, 3H), 2.34 (s, 3H), 2.89-2.83 (m, 2H), 2.96- 3.01 (m, 1H), 3.11 (t, J = 7.20 Hz, 2H), 3.18 (t, J = 7.20 Hz, 2H), 7.25 (t, J = 8.00 Hz, 1H), 7.43 (d, J = 7.65 Hz, 1H), 7.93 (d, J = 7.88 Hz, 1H), 9.01 (brs, 1H) | 409 | 407 | |
| F-100 | | (400 MHz, DMSO-D6) 0.92 (d, J = 6.18 Hz, 6H), 0.98-1.03 (m, 2H), 1.07-1.11 (m, 2H), 1.58- 1.61 (m, 3H), 2.73 (t, J = 7.83 Hz, 2H), 2.94 (t, J = 6.84 Hz, 2H), 3.03 (t, J = 6.73 Hz, 2H), 3.10-3.13 (m, 1H), 7.17 (td, J = 8.27, 1.54 Hz, 1H), 7.29-7.33 (m, 1H), 7.84 (t, J = 6.95 Hz, 1H), 10.03 (s, 1H) | 379 | 377 | |
| F-101 | | (400 MHz, DMSO-D6) 0.92 (d, J = 6.40 Hz, 6H), 0.97-0.99 (m, 2H), 1.06-1.11 (m, 2H), 1.58- 1.61 (m, 3H), 2.06 (d, J = 1.98 Hz, 3H), 2.73 (t, J = 7.72 Hz, 2H) 2.89 (t, J = 7.17 Hz, 2H), 3.03 (t, J = 7.17 Hz, 2H), 3.10-3.13 (m, 1H), 6.97 (t, J = 8.71 Hz, 1H), 7.17 (dd, J = 14.67, 8.05 Hz, 1H), 7.25 (d, J = 7.50 Hz, 1H), 9.58 (s, 1H) | 359 | 357 | |
| F-102 | | (400 MHz, CDCl3) 0.73-1.83 (m, 27H), 2.75-2.87 (m, 2H), 2.93- 2.99 (m, 1H), 3.09-3.13 (m, 2H), 3.35-4.00 (m, 1H), 6.02-6.40 (m, 1H) | 347 | 345 | Mixtures of four stereoisomers (Stereochemistry on Cyclohexane ring: Mixture of Trans-isomer and Cis-isomer) |

TABLE 156

| | | | MS | | Information |
|---|---|---|---|---|---|
| Example | Chemical structure | NMR | M + H | M − H | of structure |
| F-103 | | (400 MHz, DMSO-D6) 0.93 (d, J = 5.80 Hz, 6H), 0.95-0.99 (m, 2H), 1.05-1.09 (m, 2H), 1.59- 1.63 (m, 3H), 2.59 (t, J = 7.65 Hz, 2H), 2.69-2.75 (m, 2H), 2.88- 2.96 (m, 2H), 3.09-3.14 (m, 2H), 4.59-4.63 (m, 1H), 5.47 (d, J = 4.41 Hz, 1H), 7.23-7.28 (m, 1H), 7.32-7.35 (m, 4H), 8.05 (t, J = 6.72 Hz, 1H) | 371 | 369 | |
| F-104 | | (400 MHz, CDCl3) 0.94-0.99 (m, 8H), 1.18-1.24 (m, 2H), 1.64- 1.68 (m, 3H), 2.28 (s, 3H), 2.78- 2.80 (m, 2H), 2.95-3.00 (m, 1H), 3.05 (dd, J = 7.39, 4.96 Hz, 2H), 3.20 (dd, J = 7.61, 4.96 Hz, 2H), 6.72 (d, J = 7.28 Hz, 1H), 6.86 (d, J = 7.72 Hz, 1H), 7.04 (t, J = 7.83 Hz, 1H), 8.63 (s, 1H) | 357 | 355 | |
| F-105 | | (400 MHz, CDCl3) 0.93-0.99 (m, 2H), 0.95 (t, J = 7.39 Hz, 3H), 1.16- 1.22 (m, 2H), 1.42 (td, J = 14.89, 7.39 Hz, 2H), 1.69-1.82 (m, 2H), 2.28 (s, 3H), 2.75-2.83 (m, 2H), 2.97 (tt, J = 7.52, 3.96 Hz, 1H), 3.05- 3.12 (m, 2H), 3.13-3.20 (m, 2H), 7.07 (t, J = 7.94 Hz, 1H), 7.16 (d, J = 7.94 Hz, 1H), 7.62 (d, J = 7.94 Hz, 1H), 8.91 (brs, 1H) | 361 | 359 | |
| F-106 | | (400 MHz, CDCl3) 0.95 (d, J = 6.40 Hz, 6H) 0.96-1.00 (m, 2H), 1.17-1.24 (m, 2H), 1.62- 1.72 (m, 3H), 1.84 (s, 3H), 2.17 (s, 3H), 2.76-2.82 (m, 2H), 2.99 (tt, J = 7.12, 3.96 Hz, 1H), 3.07-3.21 (m, 4H), 10.21 (brs, 1H) | 346 | 344 | |

TABLE 157

| | | | MS | | Information |
|---|---|---|---|---|---|
| Example | Chemical structure | NMR | M + H | M − H | of structure |
| F-107 | | (400 MHz, CDCl3) 0.93-0.97 (m, 9H), 0.99 (d, 6.62 Hz, 6H), 1.16- 1.23 (m, 2H), 2.16-2.26 (m, 1H), 2.29 (s, 3H), 2.69 (d, J = 7.28 Hz 2H), 2.96 (tt, J = 7.12, 4.35 Hz, 1H), 3.05-3.12 (m, 2H), 3.14-3.21 (m, 2H), 7.07 (dd, J = 7.94, 7.72 Hz, 1H), 7.15 (d, J = 7.94 Hz, 1H), 7.63 (d, J = 7.72 Hz, 1H), 8.85 (brs, 1H) | 361 | 359 | |
| F-108 | | (400 MHz, CDCl3) 0.94-0.97 (m, 2H), 0.97 (d, J = 6.49 Hz, 6H), 1.15- 1.21 (m, 2H), 1.67-1.71 (m, 1H), 2.26 (s, 3H), 2.76-2.86 (m, 6H), 2.94-2.99 (m, 1H), 3.12 (t, J = 6.96 Hz, 2H), 3.78 (s, 3H), 4.33 (d, J = 5.57 Hz, 2H), 6.39 (brs, 1H), 6.69 (td, J = 8.35, 2.55 Hz, 2H), 7.10 (d, J = 8.35 Hz, 1H) | 385 | 383 | |
| F-109 | | (400 MHz, CDCl3) 0.97 (d, J = 6.49 Hz, 6H), 0.98-1.01 (m, 2H), 1.22 (t, J = 6.96 Hz, 2H), 1.66- 1.71 (m, 3H), 2.80-2.84 (m, 2H), 2.97-3.05 (m, 3H), 3.15-3.18 (m, 2H), 6.77 (dt, J = 8.35, 2.09 Hz, 1H), 7.33-7.35 (m, 1H), 7.38 (dt, J = 10.51, 2.15 Hz, 1H), 9.90 (s, 1H) | 379 | 377 | |
| F-110 | | (400 MHz, CDCl3) 0.95 (d, J = 6.04 Hz, 6H), 0.97-0.99 (m, 2H), 1.17-1.21 (m, 2H), 1.67- 1.71 (m, 3H), 2.27 (s, 3H), 2.77- 2.81 (m, 2H), 2.96-3.02 (m, 3H), 3.19-3.15 (m, 2H) 4.48 (d, J = 4.03 Hz, 2H), 7.13 (dd, J = 7.66, 4.84 Hz, 1H), 7.46 (d, J = 6.85 Hz, 1H), 7.61 (s, 1H), 8.38 (d, J = 4.03 Hz, 1H) | 356 | 354 | |

TABLE 158

| | | | MS | | Information |
|---|---|---|---|---|---|
| Example | Chemical structure | NMR | M + H | M − H | of structure |
| F-111 | | (400 MHz, CDCl3) 0.94-0.97 (m, 8H), 1.16-1.21 (m, 2H), 1.65- 1.70 (m, 3H), 2.27 (s, 3H) 2.76- 2.80 (m, 2H), 2.94-2.99 (m, 3H), 3.11-3.14 (m, 2H), 4.39 (d, J = 5.64 Hz, 2H), 7.09 (d, J = 5.24 Hz, 1H), 7.45 (t, J = 6.45 Hz, 1H), 8.35- 8.33 (m, 2H) | 356 | 354 | |
| F-112 | | (400 MHz, CDCl3) 0.92 (d, J = 11.69 Hz, 2H), 1.20 (dd, J = 22.05, 10.37 Hz, 4H), 1.57-1.75 (m, 7H), 1.86-1.96 (m, 2H), 2.19 (s, 3H), 2.26 (s, 3H), 2.55 (dd, J = 18.86, 8.49 Hz, 4H), 2.73 (t, J = 8.16 Hz, 2H), 3.11 (dd, J = 18.75, 6.18 Hz, 4H), 4.53 (t, J = 8.71 Hz, 1H), 6.95 (s, 2H), 7.61 (d, J = 8.60 Hz, 1H), 8.58 (s, 1H) | 409 | 407 | |
| F-113 | | (400 MHz, CDCl3) 0.95-1.04 (m, 2H), 1.11-1.19 (m, 2H), 2.30 (s, 3H), 2.97 (s, 3H), 3.05-3.10 (m, 2H), 3.13-3.22 (m, 6H), 3.96 (s, 2H),7.08 (dd, J = 8.38, 7.72 Hz, 2H), 7.15 (d, J = 7.72 Hz, 2H), 7.67 (d, J = 8.38 Hz, 1H), 8.73 (brs, 1H) | 390 | 388 | |
| F-114 | | (400 MHz, DMSO-D6) 0.92 (d, J = 6.26 Hz, 6H), 0.93-0.97 (m, 2H), 1.03-1.08 (m, 2H), 1.54- 1.64 (m, 3H), 2.14 (s, 3H), 2.61 (t, J = 7.54 Hz, 2H), 2.71 (t, J = 7.77 Hz, 2H), 2.94 (t, J = 7.54 Hz, 2H), 3.04- 3.09 (m, 1H), 4.11 (d, J = 5.33 Hz, 2H), 6.50 (dd, J = 8.23, 2.67 Hz, 1H), 6.55 (d, J = 2.55 Hz, 1H), 6.96 (d, J = 8.12 Hz, 1H), 8.13 (t, J = 5.57 Hz, 1H), 9.14 (s, 1H) | 371 | 369 | |

TABLE 159

| | | | MS | | Information |
|---|---|---|---|---|---|
| Example | Chemical structure | NMR | M + H | M − H | of structure |
| F-115 | | (400 MHz, DMSO-D6) 0.93 (d, J = 6.03 Hz, 6H), 1.13-1.28 (m, 7H), 1.63 (t, J = 6.15 Hz, 3H), 2.81 (t, J = 7.19 Hz, 2H), 2.93 (t, J = 7.42 Hz, 2H), 3.16 (t, J = 7.07 Hz, 2H), 3.32-3.36 (m, 1H), 4.64 (d, J = 5.57 Hz, 2H), 6.54 (s, 1H), 7.40 (d, J = 7.42 Hz, 1H), 7.52 (t, J = 7.88 Hz, 1H), 7.72 (d, J = 8.35 Hz, 1H), 8.82 (t, J = 5.80 Hz, 1H), 9.53 (s, 1H) | 381 | 379 | |
| F-116 | | (400 MHz, CDCl3) 0.93-0.98 (m, 8H), 1.15-1.20 (m, 2H), 1.65- 1.72 (m, 3H), 2.32 (s, 3H), 2.77- 2.81 (m, 6H), 2.93-2.98 (m, 1H), 3.07 (t, J = 6.95 Hz, 2H), 3.41-3.46 (m, 2H), 6.42 (brs, 1H), 7.07-7.14 (m, 4H) | 369 | 367 | |
| F-117 | | (400 MHz, CDCl3) 0.81-0.97 (m, 11H), 1.19 (s, 2H), 1.62 (s, 1H), 1.94 (s, 1H), 2.27 (s, 3H), 2.58 (dd, J = 14.67, 9.59 Hz, 1H), 2.82 (dd, J = 14.78, 5.07 Hz, 1H), 2.96 (s, 1H), 3.14 (d, J = 6.62 Hz, 4H), 7.07 (t, J = 7.94 Hz, 1H), 7.16 (d, J = 7.94 Hz, 1H), 7.54 (s, 1H), 9.25 (s, 1H) | 389 | 387 | Racemic form |
| F-118 | | (400 MHz, CDCl3) 0.93-0.97 (m, 8H), 1.18 (dd, J = 12.68, 6.95 Hz, 2H), 1.65-1.71 (m, 3H), 2.16 (s, 3H), 2.28 (s, 3H), 2.76-2.80 (m, 2H), 2.84 (t, J = 6.84 Hz, 2H), 2.93- 2.99 (m, 1H), 3.11 (t, J = 6.84 Hz, 2H), 4.41 (d, J = 5.51 Hz, 2H), 6.34 (brs, 1H), 7.02-7.09 (m, 3H) | 369 | 367 | |

TABLE 160

| | | | MS | | Information |
|---|---|---|---|---|---|
| Example | Chemical structure | NMR | M + H | M − H | of structure |
| F-119 | | (400 MHz, CDCl3) 0.92-0.97 (m, 8H), 1.15-1.20 (m, 2H), 1.65- 1.70 (m, 3H), 2.24 (s, 3H), 2.29 (s, 3H), 2.76-2.80 (m, 2H), 2.84 (t, J = 6.95 Hz, 2H) 2.93-2.97 (m, 1H), 3.11 (t J = 6.95 Hz, 2H), 4.35 (d, J = 5.51 Hz, 2H), 6.37 (brs, 1H), 6.94-6.96 (m, 2H), 7.05 (d, J = 7.28 Hz, 1H) | 369 | 367 | |
| F-120 | | (400 MHz, CDCl3) 0.89 (d, J = 6.62 Hz, 6H), 0.93-0.99 (m, 2H), 1.16-1.23 (m, 2H), 1.24- 1.33 (m, 0H), 1.53-1.64 (m, 1H), 1.74-1.84 (m, 2H), 2.29 (s, 3H), 2.77 (t, J = 7.83 Hz, 2H) 2.97 (tt, J = 7.12, 3.96 Hz, 1H), 3.04-3.12 (m, 2H), 3.13-3.21 (m, 2H), 7.08 (dd, J = 7.94, 7.72 Hz, 1H), 7.16 (d, J = 7.94 Hz, 1H), 7.63 (d, J = 7.72 Hz, 1H), 8.82 (brs, 1H) | 389 | 387 | |
| F-121 | | (400 MHz, CDCl3) 0.91-0.96 (m, 2H), 0.96 (d, J = 5.73 Hz, 6H), 1.13- 1.20 (m, 2H), 1.63-1.69 (m, 3H), 2.74-2.80 (m, 2H), 2.89 (t, J = 6.95 Hz, 2H), 2.94 (tt, J = 6.77, 3.96 Hz, 1H), 3.10 (t, J = 6.95 Hz, 2H), 4.49 (d, J = 6.18 Hz, 2H), 6.94 (brs, 1H), 7.15-7.22 (m, 2H), 7.28- 7.35 (m, 2H) | 375 | 373 | |
| F-122 | | (400 MHz, CDCl3) 0.90-0.95 (m, 2H), 0.97 (d, J = 5.51 Hz, 6H), 1.14- 1.21 (m, 2H), 1.63-1.69 (m, 3H), 2.73-2.80 (m, 2H), 2.88 (t, J = 6.73 Hz, 2H), 2.94 (tt, J = 6.73, 3.96 Hz, 1H), 3.09 (t, J = 6.73 Hz, 2H), 4.36 (d, J = 5.95 Hz, 2H), 7.15 (d, J = 8.27 Hz, 2H), 7.19 (brs, 1H), 7.24 (d, J = 8.27 Hz, 2H) | 375 | 373 | |

TABLE 161

| | | | MS | | Information |
|---|---|---|---|---|---|
| Example | Chemical structure | NMR | M + H | M − H | of structure |
| F-123 | | (400 MHz, CDCl3) 0.90-0.95 (m, 2H), 0.96 (d, J = 5.51 Hz, 6H), 1.13- 1.21 (m, 2H), 1.62-1.70 (m, 3H), 2.29 (s, 3H), 2.73-2.79 (m, 2H), 2.88 (t, J = 6.62 Hz, 2H), 2.94 (tt, J = 6.73, 3.96 Hz, 1H), 3.08 (t, J = 6.62 Hz, 2H), 4.40 (d, J = 5.73 Hz, 2H), 6.98 (brs, 1H), 7.01-7.11 (m, 2H), 7.23-7.27 (m, 1H) | 389 | 387 | |
| F-124 | | (400 MHz, DMSO-D6) 0.90-0.99 (m, 8H), 1.03-1.11 (m, 2H), 1.11- 1.20 (m, 3H), 1.53-1.68 (m, 3H), 2.53-2.61 (m, 2H), 2.61-2.68 (m, 2H), 2.68-2.75 (m, 2H), 2.93- 3.01 (m, 2H), 3.04-3.12 (m, 1H), 4.19-4.25 (m, 2H), 7.09-7.18 (m, 4H), 8.34-8.43 (m, 1H) | 369 | 367 | |
| F-125 | | (400 MHz, DMSO-D6) 0.86-0.99 (m, 8H), 1.01-1.10 (m, 2H), 1.10- 1.19 (m, 3H), 1.52-1.68 (m, 3H), 2.56-2.76 (m, 6H), 2.92-3.00 (m, 2H), 3.02-3.12 (m, 1H), 4.23- 4.31 (m, 2H), 7.09-7.23 (m, 4H), 8.27-8.36 (m, 1H) | 369 | 367 | |
| F-126 | | (400 MHz, DMSO-D6) 0.88-1.00 (m, 8H), 1.02-1.11 (m, 2H), 1.53- 1.66 (m, 3H), 2.14 (s, 3H), 2.63- 2.77 (m, 4H), 2.92-3.01 (m, 2H), 3.02-3.12 (m, 1H) 4.22-4.29 (m, 2H), 6.98-7.09 (m, 2H), 7.11- 7.20 (m, 1H), 8.30-8.40 (m, 1H) | 373 | 371 | |

TABLE 162

| | | | MS | | Information |
|---|---|---|---|---|---|
| Example | Chemical structure | NMS | M + H | M − H | of structure |
| F-127 | | (400 MHz, DMSO-D6) 0.89-1.00 (m, 8H), 1.02-1.12 (m, 2H), 1.51- 1.65 (m, 2H), 2.07 (s, 3H), 2.61- 2,68 (m, 2H), 2.68-2.75 (m, 2H), 2.92-3.00 (m, 2H), 3.03-3.13 (m, 1H), 3.14-3.20 (m, 1H), 3.76 (s, 3H), 4.18-4.26 (m, 2H), 6.78- 6.84 (m, 1H), 6.84-6.90 (m, 1H), 7.06-7.13 (m, 1H), 8.22-8.30 (m, 1H) | 385 | 383 | |
| F-128 | | (400 MHz, DMSO-D6) 0.94 (d, J = 5.73 Hz, 6H), 1.17-1.21 (m, 4H), 1.63 (t, J = 6.62 Hz, 3H), 2.78 (t, J = 6.95 Hz, 2H), 2.94 (t, J = 7.64 Hz, 2H), 3.18 (t, J = 6.84 Hz, 2H), 3.34-3.37 (m, 1H), 4 58 (d, J = 5.73 Hz, 2H), 6.97 (d, J = 6.84 Hz, 1H), 7.28 (t, J = 7.39 Hz, 1H), 7.43 (d, J = 8.60 Hz, 1H), 8.08 (s, 1H), 8.65 (t, J = 6.00 Hz 1H) | 381 | 379 | |
| F-129 | | (400 MHz, CDCl3) 0.93-0.99 (m, 8H), 1.14-1.22 (m, 2H), 1.66- 1.71 (m, 1H), 2.76-2.83 (m, 3H), 2.88-2.99 (m, 3H), 3.12 (t, J = 6.61 Hz, 2H), 3.50 (s, 1H), 4.46 (d, J = 5.80 Hz, 2H), 7.19-7.24 (m, 1H), 7.33 (d, J = 8.12 Hz, 2H), 7.54 (d, J = 8.12 Hz, 2H) | 409 | 407 | |
| F-130 | | (400 MHz, CDCl3) 0.95-0.98 (m, 8H), 1.16-1.21 (m, 2H), 1.67- 1.71 (m, 2H), 2.77-2.83 (m, 3H), 2.90 (t, J = 6.84 Hz, 2H), 2.94-3.00 (m, 1H), 3.13 (t, J = 6.84 Hz, 2H), 4.61 (d, J = 6.26 Hz, 2H), 6.75-6.79 (m, 1H), 7.32-7.36 (m, 1H), 7.47- 7.52 (m, 2H), 7.63 (d, J = 8.12 Hz, 1H) | 409 | 407 | |

TABLE 163

| | | | MS | | Information |
|---|---|---|---|---|---|
| Example | Chemical structure | NMR | M + H | M − H | of structure |
| F-131 | | (400 MHz, CDCl3) 0.89-1.01 (m, 2H), 0.95 (d, J = 5.60 Hz, 6H), 1.20 (dd, J = 12.80, 7.20 Hz, 2H), 1.58- 1.73 (m, 3H), 2.24 (t, 3H), 2.73- 2.86 (m, 2H), 2.93-3.02 (m, 1H), 3.02-3.23 (m, 4H), 6.97 (dd, J = 8.00, 0.80 Hz, 1H), 7.05 (d, J = 8.00 Hz, 1H), 8.01 (s, 1H), 8.82 (brs, 1H) | 375 | 373 | |
| F-132 | | (400 MHz, CDCl3) 0.92-1.00 (m, 8H), 1.16-1.25 (m, 5H). 1.63- 1.73 (m, 3H), 2.61 (q, J = 7.57 Hz, 2H), 2.76-2.84 (m, 2H), 2.94- 3.05 (m, 3H), 3.13-3.20 (m, 2H), 6.88-6.94 (m, 1H), 7.16-7.22 (m, 1H), 7.32-7.41 (m, 2H), 8.87 (brs, 1H) | 355 | 353 | |
| F-133 | | (400 MHz, DMSO-D6) 0.87-0.99 (m, 8H), 1.02-1.11 (m, 2H), 1.53- 1.65 (m, 3H), 2.03 (s, 3H), 2.62- 2.69 (m, 2H), 2.69-2.76 (m, 2H), 2.91-3.01 (m, 2H), 3.04-3.13 (m, 1H), 4.16-4.22 (m, 2H) 6.61- 6.68 (m, 1H), 6.68-6.74 (m, 1H), 6.87-6.95 (m, 1H), 8.18-8.25 (m, 1H), 9.20 (brs, 1H) | 371 | 369 | |
| F-134 | | (400 MHz, CDCl3) 0.92 (d, J = 5.95 Hz, 6H), 0.95-1.02 (m, 2H), 1.16-1.23 (m, 2H), 1.57- 1.68 (m, 3H), 2.72-2.82 (m, 2H), 2.99 (tt, J = 7.12, 3.56 Hz, 1H), 3.15- 3.25 (m, 4H), 6.73 (dd, J = 6.62, 5.94 Hz, 1H), 7.11 (dd, J = 8.91, 5.94 Hz, 1H), 7.53 (d, J = 8.91 Hz, 1H), 7.54 (s, 1H), 7.90 (d, J = 6.62 Hz, 1H), 9.92 (brs, 1H) | 367 | 365 | |

TABLE 164

| | | | MS | | Information |
|---|---|---|---|---|---|
| Example | Chemical structure | NMR | M + H | M − H | of structure |
| F-135 | | (400 MHz, CDCl3) 0.94-1.95 (m, 16H), 2.27-2.28 (m, 3H), 2.86- 2.96 (m, 2H), 3.09-3.11 (m, 2H), 3.17-3.20 (m, 2H), 7.08 (t, J = 8.00 Hz, 1H), 7.17 (d, J = 7.88 Hz, 1H), 7.63 (d, J = 7.42 Hz, 1H), 8.91 (s, 1H) | 401 | 399 | Mixtures of four stereoisomers (Stereochemistry on Cyclohexane ring: Mixture of Trans-isomer and Cis-isomer) |
| F-136 | | (400 MHz, CDCl3) 0.95-0.98 (m, 8H), 1.16-1.21 (m, 2H), 1.64- 1.70 (m, 3H), 2.25 (s, 3H), 2.78- 2.81 (m, 2H), 2.94-2.99 (m, 1H), 3.05-3.09 (m, 2H) 3.14-3.18 (m, 2H), 3.34 (s, 3H), 4.37 (s, 2H), 7.13-7.11 (m, 2H), 7.84 (d, J = 8.87 Hz, 1H), 8.46 (s, 1H) | 385 | 383 | |
| F-137 | | (400 MHz, CDCl3) 0.95-0.98 (m, 8H), 1.17-1.22 (m, 2H), 1.64- 1.70 (m, 3H), 2.22 (s, 3H), 2.78- 2.82 (m, 2H), 2.94-3.00 (m, 1H), 3.04-3.09 (m, 2H), 3.13-3.18 (m, 2H), 6.82-6.87 (m, 2H), 7.68 (dd, J = 7.05, 3.53 Hz, 1H) 6.55 (s, 1H) | 359 | 357 | |
| F-138 | | (400 MHz, CDCl3) 0.95-0.98 (m, 8H), 1.17-1.22 (m, 2H), 1.63- 1.69 (m, 3H), 2.24 (s, 3H), 2.78- 2.82 (m, 2H), 2.94-2.99 (m, 1H), 3.04-3.08 (m, 2H), 3.13-3.17 (m, 2H), 7.13-7.11 (m, 2H), 7.82 (d, J = 9.27 Hz, 1H), 8.74 (s, 1H) | 375 | 373 | |
| F-139 | | (400 MHz, CDCl3) 0.83-0.96 (m, 6H), 0.96-1.02 (m, 2H), 1.15- 1.23 (m, 2H), 1.57-1.66 (m, 3H), 2.74-2.82 (m, 2H), 2.97-3.05 (m, 1H), 3.19-3.30 (m, 2H), 3.30- 3.41 (m, 2H), 6.73-6.81 (m, 1H), 7.16-7.23 (m, 1H), 7.61-7.68 (m, 1H), 7.88-7.98 (m, 1H), 11.41 (brs, 1H) | 368 | 366 | |

TABLE 165

| | | | ms | | Information |
|---|---|---|---|---|---|
| Example | Chemical structure | NMR | M + H | M − H | of structure |
| F-140 | | (400 MHz, CDCl3) 0.90-1.04 (m, 2H), 0.95 (d, J = 6.00 Hz, 6H), 1.20 (dd, J = 12.92, 7.00 Hz, 2H), 1.58-1.77 (m, 3H), 2.22 (s, 3H), 2.74-2.87 (m, 2H), 2.93-3.02 (m, 1H), 3.03-3.24 (m, 4H), | 420 | 418 | |
| | | 6.99 (d, J = 8.24 Hz, 1H), | | | |
| | | 7.13 (d, J = 8.24 Hz, | | | |
| | | 1H), 8.13 (s, 1H), | | | |
| | | 8.87 (brs, 1H) | | | |
| F-141 | | (400 MHz, CDCl3) 0.85-1.02 (m, 3H), 1.05-1.38 (m, 9H), 1.56-1.93 (m, 7H), 2.18 (s, 3H), 2.26 (s, 3H), 2.57-2.66 (m, 1H), 2.70-2.82 (m, 2H), 2.99-3.19 (m, 4H), 6.91-7.01 (m, 2H), | 409 | 407 | Racemic form |
| | | 7.59 (d, J = 8.68 Hz, | | | |
| | | 1H), 8.43 (s, 1H) | | | |
| F-142 | | (400 MHz, CDCl3) 0.87-1.01 (m, 2H), 1.09-1.40 (m, 4H), 1.61-1.80 (m, 7H), 2.13 (s, 3H), 2.26 (s, 3H), 2.67 (t, J = 8.32 Hz, 2H), 3.00-3.13 (m, 4H), 4.57 (dd, J = 16.44, 8.12 Hz, 2H), | 437 | 435 | |
| | | 6.92-7.00 (m, 2H), | | | |
| | | 7.42 (d, J = 7.88 Hz, | | | |
| | | 1H), 7.73 (s, 1H) | | | |
| F-143 | | (400 MHz, CDCl3) 0.91-1.02 (m, 2H), 0.96 (d, J = 5.12 Hz, 6H), 1.21 (dd, J = 12.96, 6.72 Hz, 2H), 1.60-1.72 (m, 3H), 2.37 (s, 3H), 2.75-2.86 (m, 2H), 2.94-3.03 (m, 1H), 3.05-3.21 (m, 4H), 7.22 (d, J = 7.84 Hz, 1H), 7.28 (d, J = 7.84 Hz, 1H), | 366 | 364 | |
| | | 8.36 (s, 1H), 9.32 (brs, 1H) | | | |

TABLE 166

| | | | ms | | Information |
|---|---|---|---|---|---|
| Example | Chemical structure | NMR | M + H | M − H | of structure |
| F-144 | | (400 MHz, CDCl3) 1.12-1.30 (m, 8H), 1.53 (s, 1H), 1.70 (d, J = 4.63 Hz, 2H), 1.85-1.96 (m, 3H), 2.28 (s, 3H), 3.11 (t, J = 7.39 Hz, 3H), 3.18 (d, J = 6.84 Hz, 2H), 3.36 (s, 1H), 4.66 (s, 2H), 7.07 (t, J = 7.94 Hz, | 417 | 415 | |
| | | 1H), 7.16 (d, J = 7.94 Hz, | | | |
| | | 1H), 7.57 (d, J = 7.94 Hz, | | | |
| | | 1H), 9.00 (s, 1H) | | | |
| F-145 | | (400 MHz, CDCl3) 0.95-1.01 (m, 8H), 1.25 (s, 2H), 1.67 (t, J = 6.06 Hz, 3H), 2.13 (s, 3H), 2.25 (s, 3H), 2.77-2.84 (m, 2H), 3.01 (s, 1H), 3.31 (t, J = 10.92 Hz, 1H), 3.73 (d, J = 8.82 Hz, 1H), 3.95 (d, J = 8.16 Hz, 1H), 4.20 (dd, J = 19.08, 12.68 Hz, 2H), 4.45 (t, J = 9.92 Hz, 1H), 7.07 (t, J = 8.05 Hz, 1H), 7.16 (t, J = 8.38 Hz, 1H), | 458 | 456 | Racemic form |
| | | 7.73 (d, J = 7.94 Hz, 1H), | | | |
| | | 9.47 (s, 1H) | | | |
| F-146 | | (400 MHz, CDCl3) 0.94 (t, J = 6.18 Hz, 8H), 1.26 (s, 2H), 1.66 (t, J = 7.17 Hz, 3H), 1.84 (s, 2H), 2.22 (s, 2H), 2.77-2.84 (m, 2H), 3.01 (s, 1H), 3.41 (t, J = 11.14 Hz, 2H), 3.81 (dd, J = 21.61, 9.26 Hz, 1H), 4.04 (t, J = 10.48 Hz, 1H), 4.19 (s, 2H), 4.26 (t, J = 10.03 Hz, 1H), 4.48 (t, J = 9.81 | 474 | 472 | Racemic form |
| | | Hz, 1H), 7.07 (t, J = 8.16 Hz, | | | |
| | | 1H), 7.16 (d, J = 8.16 Hz, | | | |
| | | 1H), 7.67 (d, J = 8.16 Hz, | | | |
| | | 1H), 9.50 (s, 1H) | | | |

TABLE 167

| | | | ms | | Information |
|---|---|---|---|---|---|
| Example | Chemical structure | NMR | M + H | M − H | of structure |
| F-147 | | (400 MHz, CDCl3) 0.92-0.99 (m, 8H), 1.14-1.27 (m, 5H), 1.62-1.72 (m, 3H), 2.15 (s, 3H), 2.63 (q, J = 7.57 Hz, 2H), 2.76-2.83 (m, 2H), 2.94-3.00 (m, 1H), | 369 | 367 | |
| | | 3.05-3.10 (m, 2H), | | | |
| | | 3.15-3.21 (m, 2H), | | | |
| | | 6.97 (d, J = 7.50 Hz, | | | |
| | | 1H), 7.09 (t, J = 7.83 Hz, | | | |
| | | 1H), 7.50 (d, J = 7.94 Hz, | | | |
| | | 1H), 8.32 (brs, 1H) | | | |
| F-148 | | (400 MHz, CDCl3) 0.89-0.95 (m, 2H), 0.96 (d, J = 6.40 Hz, 6H), 1.12-1.18 (m, 2H), 1.64-1.70 (m, 3H), 2.33 (s, 3H), 2.73-2.78 (m, 2H), 2.80 (t, J = 7.23 Hz, 2H), 2.92 (tt, J = 7.12, 4.35 Hz, 1H), 3.09 (t, J = | 385 | 383 | |
| | | 7.23 Hz, 2H), 3.83 (s, 3H), | | | |
| | | 4.35 (d, J = 5.95 Hz, 2H), | | | |
| | | 6.44 (brs, 1H), 6.67 (s, 1H), | | | |
| | | 6.70 (d, J = 7.72 Hz, 1H), | | | |
| | | 7.08 (d, J = 7.50 Hz, 1H) | | | |
| F-149 | | (400 MHz, CDCl3) 0.69-0.95 (m, 2H), 0.97 (d, J = 5.73 Hz, 6H), 1.14-1.21 (m, 2H), 1.63-1.72 (m, 3H), 2.25 (s, 3H), 2.72-2.78 (m, 2H), 2.85 (t, J = 6.95 Hz, 2H), 2.92 (tt, J = 7.12, 3.96 Hz, 1H), 3.07 (t, J = | 371 | 369 | |
| | | 6.95 Hz, 2H), 4.24 (d, J = | | | |
| | | 6.40 Hz, 2H), 6.58 (d, J = | | | |
| | | 7.50 Hz, 1H), 6.72 (s, 1H), | | | |
| | | 6.95 (d, J = 7.50 Hz, 1H), | | | |
| | | 8.31 (brs, 1H), 9.52 (brs, 1H) | | | |

TABLE 168

| | | | ms | | Information |
|---|---|---|---|---|---|
| Example | Chemical structure | NMR | M + H | M − H | of structure |
| F-150 | | (400 MHz, DMSO-D6) 0.87-1.02 (m, 8H), 1.04-1.09 (m, 2H), 1.60 (tt, J = 13.91, 4.83 Hz, 3H), 2.69-2.74 (m, 4H), 2.99 (t, J = 7.42 Hz, 2H), 3.06-3.11 (m, 1H), 4.56 (d, J = 5.80 Hz, 2H), 7.05 (dd, J = 7.88, | 381 | 379 | |
| | | 7.19 Hz, 1H), 7.16 | | | |
| | | (d, J = 6.96 Hz, 1H), | | | |
| | | 7.64 (d, J = 7.88 Hz, 1H), | | | |
| | | 8.08 (d, J = 1.39 Hz, | | | |
| | | 1H), 8.55 (t, J = 5.80 Hz, | | | |
| | | 1H), 12.99 (brs, 1H) | | | |
| F-151 | | (400 MHz, CDCl3) 0.91-1.00 (m, 2H), 0.95 (d, J = 6.48 Hz, 6H), 1.16-1.24 (m, 2H), 1.59-1.71 (m, 3H), 2.34 (s, 3H), 2.76-2.84 (m, 2H), 2.94-3.01 (m, 1H), 3.06-3.21 (m, 4H), 7.23-7.29 (m, 2H), 8.28 (s, 1H), 9.04 (brs, 1H) | 409 | 407 | |
| F-152 | | (400 MHz, CDCl3) 0.60-0.69 (m, 2H), 0.82-1.04 (m, 4H), 0.95 (d, J = 6.44 Hz, 6H), 1.15-1.23 (m, 2H), 1.57-1.74 (m, 3H), 1.80-1.89 (m, 1H), 2.19 (s, 3H), 2.74-2.85 (m, 2H), 2.93-3.00 (m, 1H), 3.02-3.22 (m, 4H), 6.74 (d, | 381 | 379 | |
| | | J = 8.04 Hz, 1H), 7.01 (d, J = | | | |
| | | 8.04 Hz, 1H), 7.61 (s, | | | |
| | | 1H), 8.30 (brs, 1H) | | | |
| F-153 | | (400 MHz, CDCl3) 0.89-0.99 (m, 2H), 0.92 (t, J = 7.20 Hz, 3H), 0.95 (d, J = 6.48 Hz, 6H), 1.15-1.22 (m, 2H), 1.54-1.72 (m, 5H), 2.20 (s, 3H), 2.53 (t, J = 7.44 Hz, 2H), 2.75-2.83 (m, 2H), 2.93-3.00 (m, 1H), 3.03-3.10 (m, 2H), 3.12-3.20 (m, 2H), 6.85 (d, J = 7.16 Hz, | 383 | 381 | |
| | | 1H), 7.04 (d, J = 7.64 Hz, | | | |
| | | 1H), 7.69 (s, 1H), | | | |
| | | 8.30 (brs, 1H) | | | |

TABLE 169

| | | | ms | | Information |
|---|---|---|---|---|---|
| Example | Chemical structure | NMR | M + H | M − H | of structure |
| F-154 | | (400 MHz, CDCl3) 0.91-1.02 (m, 2H), 0.95 (d, J = 6.28 Hz, 6H), 1.17-1.24 (m, 2H), 1.60-1.70 (m, 3H), 2.33 (s, 3H), 2.74-2.83 (m, 2H), 2.93-3.04 (m, 1H), 3.05-3.21 (m, 4H), 7.21-7.28 (m, 1H), 7.54-7.63 (m, 1H), 8.25 (s, 1H), | 384 | 382 | |
| | | 8.99 (brs, 1H) | | | |
| F-155 | | (400 MHz, DMSO-D6) 0.92-0.98 (m, 4H), 1.13-1.26 (m, 6H), 1.57-1.76 (m, 7H), 1.99 (d, J = 12.35 Hz, 3H), 2.11 (d, J = 3.31 Hz, 3H), 2.75 (t, J = 6.95 Hz, 2H), 3.10 (s, 1H), 3.45-3.54 (m, 1H), 3.73 (t, J = 8.71 Hz, 1H), 3.85-4.11 (m, 4H), 7.17 (t, J = 8.05 Hz, 1H), 7.28- | 498 | 496 | Racemic form |
| | | 7.31 (m, 2H), 9.89 | | | |
| | | (d, J = 10.14 Hz, 1H) | | | |
| F-156 | | (400 MHz, CDCl3) 0.57-0.61 (m, 2H), 0.87-0.92 (m, 2H), 0.94-1.00 (m, 8H), 1.16-1.22 (m, 2H), 1.64-1.72 (m, 3H), 1.82-1.90 (m, 1H), 2.30 (s, 3H), 2.77-2.83 | 381 | 379 | |
| | | (m, 2H), 2.94-3.00 (m, 1H), | | | |
| | | 3.05-3.10 (m, 2H), | | | |
| | | 3.16-3.21 (m, 2H), | | | |
| | | 6.87 (d, J = 7.65 Hz, | | | |
| | | 1H), 7.07 (t, J = 7.88 Hz, | | | |
| | | 1H), 7.54 (d, J = 7.88 Hz, | | | |
| | | 1H), 8.28 (brs, 1H) | | | |
| F-157 | | (400 MHz, DMSO-D6) 0.92 (d, J = 6.18 Hz, 6H), 0.96-1.01 (m, 2H), 1.07-1.12 (m, 2H), 1.56- 1.65 (m, 3H), 2.24 (s, 3H), 2.70-2.77 (m, 2H), 2.88-2.95 (m, 2H), 3.02-3.08 (m, 2H), 3.10-3.16 (m, 1H), | 384 | 382 | |
| | | 7.22 (brs, 1H), 7.58 (d, | | | |
| | | J = 7.94 Hz, 2H), 7.65 (d, | | | |
| | | J = 7.94 Hz, 2H), 7.71 | | | |
| | | (s, 1H), 7.83 (brs, 1H), | | | |
| | | 9.47 (brs, 1H) | | | |

TABLE 170

| | | | ms | | Information |
|---|---|---|---|---|---|
| Example | Chemical structure | NMR | M + H | M − H | of structure |
| F-158 | | (400 MHz, CDCl3) 1.09-1.20 (m, 4H), 1.30 (s, 3H), 1.32 (d, J = 4.87 Hz, 3H), 2.28 (d, J = 16.23 Hz, 3H), 2.82-2.89 (m, 1H), 3.08 (tt, J = 9.04, 3.71 Hz, 3H), 3.19 (t, J = 6.15 | 371 | 369 | |
| | | Hz, 2H), 7.09 (t, J = 8.12 | | | |
| | | Hz, 1H), 7.18 (d, J = 7.88 | | | |
| | | Hz, 1H), 7.57 (t, J = 15.19 | | | |
| | | Hz, 1H), 8.58 (s, 1H) | | | |
| F-159 | | (400 MHz, CDCl3) 0.95 (d, J = 6.49 Hz, 6H), 1.15-1.27 (m, 3H), 1.62-1.72 (m, 2H), 2.78- 2.86 (m, 2H), 3.01-3.10 (m, 1H), 3.29-3.38 (m, 1H), 3.39-3.47 (m, 1H), 3.47-3.51 (m, 2H), 4.10-4.23 (m, 2H), 4.66 (dd, J = 9.86, 4.75 Hz, 1H), 7.06 (t, J = 8.12 Hz, 1H), 7.17 (d, J = 8.12 Hz, 1H), | 447 | 445 | Racemic form |
| | | 7.45 d, J = 8.12 Hz, | | | |
| | | 1H), 8.14 (brs, 1H) | | | |
| F-160 | | (400 MHz, CDCl3) 0.73-0.82 (m, 1H), 0.86-1.01 (m, 2H), 0.91 (d, J = 6.24 Hz, 3H), 1.09-1.42 (m, 6H), 1.58-1.80 (m, 7H), 2.18 (s, 3H), 2.26 (s, 3H), 2.71-2.83 (m, 2H), 2.96-3.20 (m, 5H), | 425 | 423 | Racemic form |
| | | 6.92-6.99 (m, 2H), | | | |
| | | 7.60 (d, J = 8.56 Hz, | | | |
| | | 1H), 8.27 (s, 1H) | | | |
| F-161 | | (400 MHz, CDCl3) 0.92-1.00 (m, 8H), 1.18-1.24 (m, 2H), 1.61-1.71 (m, 3H), 2.36 (s, 3H), 2.77-2.83 (m, 2H), 2.94-3.00 (m, 1H), 3.07-3.19 (m, 4H), 7.42 (s, 1H), | 366 | 364 | |
| | | 7.45 (d, J = 8.58 | | | |
| | | Hz, 1H), 8.25 (d, | | | |
| | | J = 8.58 Hz, 1H), | | | |
| | | 9.50 (brs, 1H) | | | |

TABLE 171

| | | | ms | | Information |
|---|---|---|---|---|---|
| Example | Chemical structure | NMR | M + H | M − H | of structure |
| F-162 | | (400 MHz, CDCl3) 0.96-0.99 (m, 8H), 1.24 (t, J = 5.62 Hz, 3H), 1.67 (d, J = 8.16 Hz, 3H), 2.13 (s, 3H), 2.14 (s, 3H), 2.25 (s, 3H), 2.77-2.84 (m, 2H), 2.99 (d, J = 3.97 Hz, 1H), 3.30 (t, J = 11.03 Hz, 1H), 3.75 (d, J = 8.60 Hz, 1H), 3.94 (t, J = 7.72 Hz, 1H), 4.20 (t, J = 6.29 Hz, 2H), 4.43 (dd, J = | 438 | 436 | Racemic form |
| | | 11.47, 8.38 Hz, 1H), | | | |
| | | 6.95 (d, J = 9.04 Hz, | | | |
| | | 2H), 7.71 (d, J = 7.94 Hz, | | | |
| | | 1H), 9.05 (s, 1H) | | | |
| F-163 | | (400 MHz, CDCl3) 1.02-1.06 (m, 2H), 1.12-1.19 (m, 11H), 2.28 (s, 3H), 3.03-3.15 (m, 7H), 3.77 (t, J = 6.62 Hz, 2H), 7.08 (d, J = 7.94 Hz, 1H), 7.15 (d, J = 7.94 Hz, 1H), 7.61 (d, J = | 405 | 403 | |
| | | 8.16 Hz, 1H), 8.88 (s, 1H) | | | |
| F-164 | | (400 MHz, DMSO-D6) 0.86-1.03 (m, 1H), 0.93 (d, J = 6.04 Hz, 6H), 1.05-1.17 (m, 3H), 1.54-1.69 (m, 3H), 2.16 (s, 3H), 2.76 (t, J = 7.56 Hz, 2H), 3.02 (dd, J = 15.16, 7.60 Hz, 1H), | 391 | 389 | Racemic form |
| | | 3.08-3.17 (m, 2H), | | | |
| | | 5.26-5.35 (m, 1H), | | | |
| | | 5.68 (d, J = 6.72 Hz, 1H), | | | |
| | | 7.12-7.20 (m, 1H), 7.23-7.32 | | | |
| | | (m, 2H), 9.68 (s, 1H) | | | |
| F-165 | | (400 MHz, CDCl3) 0.89-1.01 (m, 2H), 0.96 (d, J = 6.48 Hz, 6H), 1.16-1.23 (m, 2H), 1.59-1.71 (m, 3H), 2.35 (s, 3H), 2.74-2.84 (m, 2H), 2.93-3.01 (m, 1H), 3.03-3.20 (m, 4H), 7.26 (d, | 409 | 407 | |
| | | J = 8.68 Hz, 1H), 7.63 | | | |
| | | (d, J = 8.68 Hz, 1H), | | | |
| | | 9.06 (s, 1H) | | | |

TABLE 172

| | | | ms | | Information |
|---|---|---|---|---|---|
| Example | Chemical structure | NMR | M + H | M − H | of structure |
| F-166 | | (400 MHz, CDCl3) 1.02-1.15 (m, 4H), 2.29 (s, 3H), 3.00-3.07 (m, 1H), 3.07-3.14 (m, 2H), 3.15-3.21 (m, 2H), 4.55 (s, 2H), 4.70 (s, 2H), 7.07 (dd, J = 7.94, 7.72 Hz, 1H), | 425 | 423 | |
| | | 7.16 (d, J = 7.94 Hz, | | | |
| | | 1H), 7.27-7.38 (m, | | | |
| | | 5H), 7.60 (d, J = 7.72 | | | |
| | | Hz, 1H), 8.78 (brs, 1H) | | | |
| F-167 | | (400 MHz, CDCl3) 0.91 (dd, J = 6.26, 2.32 Hz, 6H), 0.95-1.02 (m, 1H), 1.07-1.14 (m, 1H), 1.15-1.25 (m, 2H), 1.54-1.64 (m, 3H), 2.18 (s, 3H), 2.69- 2.79 (m, 2H), 2.96-3.12 | 405 | 403 | Racemic form |
| | | (m, 3H), 3.85-4.00 (m, | | | |
| | | 3H), 7.06 (t, J = 8.00 | | | |
| | | Hz, 1H), 7.18 (d, J = | | | |
| | | 7.42 Hz, 1H), | | | |
| | | 7.38 (d, J = 8.12 Hz, | | | |
| | | 1H), 8.63 (brs, 1H) | | | |
| F-168 | | (400 MHz, CDCl3) 0.88-0.94 (m, 2H), 1.10-1.23 (m, 5H), 2.02-2.10 (m, 2H), 2.30 (s, 3H) 2.40-2.64 (m, 3H), 2.87-2.93 (m, 1H), 3.08-3.10 (m, 2H), 3.14-3.17 (m, | 373 | 371 | Mixture of two stereoisomers (Stereochemistry on Cyclobutane ring: Mixture of Trans-isomer and Cis-isomer) |
| | | 2H), 3.38-3.72 (m, | | | |
| | | 1H), 7.09 (t, J = 8.00 | | | |
| | | Hz, 1H), 7.17 (d, J = | | | |
| | | 8.12 Hz, 1H), 7.62 (d, | | | |
| | | J = 7.65 Hz, 1H), | | | |
| | | 8.90 (s, 1H) | | | |
| F-169 | | (400 MHz, CDCl3) 0.98 (s, 2H), 1.19 (t, J = 6.51 Hz, 2H), 1.45 (s, 2H), 1.59 (t, J = 5.51 Hz, 4H), 2.27 (s, 3H), 2.46 (s, 4H), 2.78 (t, J = 7.50 Hz, 2H), 3.00 | 416 | 414 | |
| | | (t, J = 7.83 Hz, 3H), | | | |
| | | 3.11-3.16 (m, 4H), | | | |
| | | 7.07 (t, J = 7.94 Hz, | | | |
| | | 1H), 7.16 (d, J = 7.94 | | | |
| | | Hz, 1H), 7.55 (d, | | | |
| | | J = 7.50 Hz, 1H), | | | |
| | | 9.11 (s, 1H) | | | |

TABLE 173

| | | | ms | | Information |
|---|---|---|---|---|---|
| Example | Chemical structure | NMR | M + H | M − H | of structure |
| F-170 | | (400 MHz, CDCl3) 0.95-1.00 (m, 2H), 1.18-1.21 (m, 2H), 1.31-1.33 (m, 2H), 1.62-1.65 (m, 3H), 1.72 (t, J = 7.72 Hz, 2H), 2.29 (s, 3H), | 416 | 414 | |
| | | 2.80 (t, J = 7.94 Hz, | | | |
| | | 2H), 2.96-2.99 (m, 1H), | | | |
| | | 3.11 (d, J = 6.40 Hz, 2H), | | | |
| | | 3.16 (d, J = 6.62 Hz, | | | |
| | | 2H), 3.37 (t, J = 10.92 Hz, | | | |
| | | 2H), 3.94-3.97 (m, 2H), | | | |
| | | 7.08 (t, J = 7.94 Hz, 1H), | | | |
| | | 7.17 (d, J = 8.16 Hz, 1H), | | | |
| | | 7.57 (s, 1H), 8.93 (s, 1H) | | | |
| F-171 | | (400 MHz, CDCl3) 0.98 (s, 2H), 1.17-1.19 (m, 8H), 2.29 (s, 3H), 3.05-3.16 (m, 11H), 7.08 (d, J = 7.94 Hz, 1H), 7.15 (d, J = 10.00 Hz, 1H), 7.55 (s, 1H), 8.81 (s, 1H) | 390 | 388 | |
| F-172 | | (400 MHz, CDCl3) 0.80-0.84 (m, 2H), 1.14 (d, J = 6.18 Hz, 2H), 2.26 (s, 3H), 2.71 (s, 1H), 3.08 (s, 2H), 3.12 (d, J = 6.40 Hz, 2H), 3.37 (t, J = 6.73 Hz, 2H), 4.63 (q, J = 6.47 Hz, | 399 | 397 | |
| | | 2H), 6.14 (dd, J = 5.18, | | | |
| | | 3.20 Hz, 1H), 7.09 (t, J = | | | |
| | | 8.05 Hz, 1H), 7.17 (d, J = | | | |
| | | 7.94 Hz, 1H), 7.30 (s, 1H), | | | |
| | | 7.49 (s, 1H), 7.61 (s, | | | |
| | | 1H), 8.67 (s, 1H) | | | |
| F-173 | | (400 MHz, CDCl3) 0.78 (s, 2H), 0.96-0.99 (m, 2H), 1.15-1.24 (m, 6H), 1.68-1.73 (m, 7H), 2.07 (d, J = 2.65 Hz, 3H), 2.32 (s, 3H), 2.73-2.80 (m, 3H), 3.27 (t, J = 11.03 Hz, 1H), 3.67-3.71 (m, 2H), 3.79 (d, J = 6.40 Hz, 3H), 3.90-3.99 (m, 2H), 4.28 (q, J = 6.40 Hz, 3H), 6.63 (t, J = 9.26 Hz, 2H), 6.97 (d, J = 7.50 Hz, 1H), 7.23 (s, 1H) | 508 | 506 | Racemic form |

TABLE 174

| | | | ms | | Information |
|---|---|---|---|---|---|
| Example | Chemical structure | NMR | M + H | M − H | of structure |
| F-174 | | (400 MHz, CDCl3) 0.95-0.99 (m, 8H), 1.16-1.21 (m, 2H), 1.64-1.70 (m, 3H), 2.24 (s, 3H), 2.78- 2.81 (m, 2H), 2.94- 3.00 (m, 1H), 3.05- 3.09 (m, 2H), 3.14- 3.18 (m, 2H), 3.35 (s, 3H), 4.40 (s, 2H), 7.02 (d, J = 5.84 Hz, 1H), 7.13 (d, J = 7.66 | 385 | 383 | |
| | | Hz, 1H), 7,84 (s, | | | |
| | | 1H), 8.45(s, 1H) | | | |
| F-175 | | (400 MHz, CDCl3) 0.92-0.99 (m, 8H), 1.08-1.12 (m, 1H), 1.18-1.21 (m, 2H), 1.59-1.65 (m, 3H), 1.94-2.01 (m, 1H), 2.05-2.12 (m, 1H), 2.17 (s, 3H), 2.76-2.80 (m, 2H), 2.92 (dd, J = | 419 | 417 | Racemic form |
| | | 14.10, 4.84 Hz, 1H), | | | |
| | | 2.98-3.04 (m, 1H), 3.16 | | | |
| | | (dd, J = 14.10, 10.07 Hz, | | | |
| | | 1H), 3.67-3.80 (m, 2H), | | | |
| | | 3.94-4.01 (m, 1H), 7.06 | | | |
| | | (dd, J = 8.87, 7.25 Hz, | | | |
| | | 1H), 7.16 (d, J = 7.66 Hz, | | | |
| | | 1H), 7.44 (d, J = 8.46 Hz, | | | |
| | | 1H), 8.25 (s, 1H) | | | |
| F-176 | | (400 MHz, CDCl3) 0.75-0.84 (m, 1H), 0.96 (d, J = 6.40 Hz, 6H), 0.98-1.04 (m, 1H), 1.06-1.15 (m, 2H), 1.58-1.70 (m, 4H), 1.83-1.94 (m, 1H), 1.95-2.07 (m, 1H), 2.32 (s, 3H), 2.55-2.66 (m, 1H), 2.66-2.73 | 429 | 427 | Racemic form |
| | | (m, 2H), 2.78-2.88 | | | |
| | | (m, 1H), 2.88-2.96 | | | |
| | | (m, 1H), 3.53-3.69 | | | |
| | | (m, 2H), 3.81 (s, 3H), | | | |
| | | 3.85-3.96 (m, 1H), | | | |
| | | 4.19-4.31 (m, 2H), | | | |
| | | 6.28-6.37 (m, 1H), | | | |
| | | 6.63 (s, 1H), 6.66 (d, | | | |
| | | J = 7.50 Hz, 1H), 6.97 | | | |
| | | (d, J = 7.50 Hz, 1H) | | | |

TABLE 175

| | | | ms | | Information |
|---|---|---|---|---|---|
| Example | Chemical structure | NMR | M + H | M − H | of structure |
| F-177 | | (400 MHz, DMSO-D6) 0.81-0.92 (m, 5H), 0.94-0.98 (m, 2H), 1.05-1.10 (m, 2H), 1.17-1.29 (m, 2H), 1.55-1.61 (m, 2H), 1.63-1.67 (m, 2H), 1.73-1.77 (m, 2H), | 429 | 427 | |
| | | 2.17 (s, 3H), 2.73 | | | |
| | | (t, J = 8.00 Hz, 2H), | | | |
| | | 2.87 (t, J = 7.19 Hz, | | | |
| | | 2H), 3.03 (t, J = 7.19 | | | |
| | | Hz, 2H), 3.07-3.13 | | | |
| | | (m, 1H), 7.16 (t, J = | | | |
| | | 7.88 Hz, 1H), 7.25 | | | |
| | | (d, J = 7.42 Hz, 1H), | | | |
| | | 7.30 (dd, J = 7.88, 1.16 | | | |
| | | Hz, 1H), 9.66 (s, 1H) | | | |
| F-178 | | (400 MHz, CDCl3) 0.97 (tt, J = 9.86, 3.40 Hz, 2H), 1.09-1.21 (m, 2H), 1.46 (dt, J = 9.35, 4.00 Hz, 9H), 2.28 (dd, J = 15.65, 9.62 Hz, 3H), 3.06-3.10 | 419 | 417 | |
| | | (m, 3H), 3.19 (t, J = | | | |
| | | 6.15 Hz, 2H), 3.86 | | | |
| | | (ddd, J = 16.99, 10.15, | | | |
| | | 4.12 Hz, 2H), 7.09 | | | |
| | | (dd, J = 18.44, 10.32 | | | |
| | | Hz, 1H), 7.16 (d, J = | | | |
| | | 7.88 Hz, 1H), 7.56 (dd, | | | |
| | | J = 18.90, 9.16 Hz, | | | |
| | | 1H), 8.88 (s, 1H) | | | |
| F-179 | | (400 MHz, CDCl3) 0.89-0.94 (m, 2H), 0.96 (d, J = 5.95 Hz, 6H), 1.12-1.18 (m, 2H), 1.63-1.71 (m, 3H), 2.73-2.78 (m, 2H), 2.82 (t, J = 7.06 Hz, 2H), 2.92 (tt, J = 7.12, | 371 | 369 | |
| | | 3.96 Hz, 1H), 3.10 | | | |
| | | (t, J = 7.17 Hz, | | | |
| | | 2H), 3.85 (s, 3H), | | | |
| | | 4.40 (d, J = 5.95 Hz, | | | |
| | | 2H), 6.53 (brs, 1H), | | | |
| | | 6.85 (d, J = 7.94 Hz, | | | |
| | | 1H), 6.88 (t, J = 7.39 Hz, | | | |
| | | 1H), 7.18-7.26 (m, 2H) | | | |

TABLE 176

| | | | ms | | Information |
|---|---|---|---|---|---|
| Example | Chemical structure | NMR | M + H | M − H | of structure |
| F-180 | | (400 MHz, CDCl3) 1.03-1.09 (m, 2H), 1.17-1.24 (m, 2H), 2.25 (s, 3H), 2.29 (s, 3H), 3.03-3.12 (m, 3H), 3.15-3.22 (m, 2H), 4.46 (s, 2H), 6.65 (d, J = 8.58 Hz, 2H), 7.02 (d, J = | 424 | 422 | |
| | | 8.58 Hz, 2H), 7.08 (dd, | | | |
| | | J = 7.88, 7.65 Hz, 1H), | | | |
| | | 7.16 (d, J = 7.88 Hz, 1H), | | | |
| | | 7.63 (d, J = 7.65 Hz, | | | |
| | | 1H), 8.53 (brs, 1H) | | | |
| F-181 | | (400 MHz, DMSO-D6) 0.87 (s, 9H), 0.94-0.98 (m, 2H), 1.05-1.10 (m, 2H), 1.22-1.27 (m, 2H), 1.67 (tt, J = 11.71, 4.10 Hz, 2H), 2.18 (s, 3H), 2.69 (t, J = 7.65 Hz, 2H), 2.88 (t, J = 7.07 | 403 | 402 | |
| | | Hz, 2H), 3.03 (t, J = 7.30 | | | |
| | | Hz, 2H), 3.07-3.13 (m, | | | |
| | | 1H), 7.16 (t, J = 7.88 Hz, | | | |
| | | 1H), 7.25 (d, J = 7.42 Hz, | | | |
| | | 1H), 7.30 (dd, J = 7.88, | | | |
| | | 1.16 Hz, 1H), 9.66 | | | |
| | | (brs, 1H) | | | |
| F-182 | | (400 MHz, CDCl3) 0.95-1.04 (m, 8H), 1.14-1.28 (m, 3H), 1.75-1.97 (m, 3H), 2.26 (s, 3H), 3.05 (tt, J = 9.26, 4.67 Hz, 3H), 3.19 (dd, J = 14.00, 8.93 Hz, 2H), 4.97 (dd, | 391 | 389 | Racemic form |
| | | J = 8.93, 4.74 Hz, 1H), | | | |
| | | 7.08 (t, J = 7.94 Hz, 1H), | | | |
| | | 7.17 (d, J = 7.94 Hz, 1H), | | | |
| | | 7.58 (d, J = 7.72 Hz, 1H), | | | |
| | | 8.58 (s, 1H) | | | |
| F-183 | | (400 MHz, CDCl3) 0.99-1.03 (m, 2H), 1.20-1.23 (m, 2H), 1.26 (t, J = 7.19 Hz, 3H), 2.28 (s, 3H), 2.95 (t, J = 6.80 Hz, 2H), 3.00-3.10 (m, 5H), 3.18 (t, J = 6.03 Hz, 2H), | 405 | 403 | |
| | | 4.15 (q, J = 7.19 Hz, 2H), | | | |
| | | 7.09 (t, J = 8.12 Hz, 1H), | | | |
| | | 7.17 (d, J = 8.35 Hz, 1H), | | | |
| | | 7.64 (d, J = 7.65 Hz, 1H), | | | |
| | | 8.68 (brs, 1H) | | | |

TABLE 177

| | | | | | Infor- |
|---|---|---|---|---|---|
| | | | ms | | mation of |
| Example | Chemical structure | NMR | M + H | M − H | structure |
| F-184 | | (400 MHz, CDCl3) 0.99-1.03 (m, 2H), 1.18-1.24 (m, 2H), 2.27 (s, 3H), 2.93 (s, 3H), 2.96 (t, J = 7.06 Hz, 2H), 3.01- 3.08 (m, 3H), 3.06 | 404 | 402 | |
| | | (s, 3H), 3.11 (t, J = | | | |
| | | 6.95 Hz, 2H), 3.17 | | | |
| | | (t, J = 6.18 Hz, | | | |
| | | 2H), 7.08 (t, J = | | | |
| | | 8.05 Hz, 1H), 7.15 | | | |
| | | (d, J = 7.72 Hz, | | | |
| | | 1H), 7.65 (d, J = | | | |
| | | 7.94 Hz, 1H), | | | |
| | | 8.73 (brs, 1H) | | | |
| F-185 | | (400 MHz, DMSO- D6) 0.97-1.01 (m, 2H), 1.07-1.12 (m, 2H), 2.19 (s, 3H), 2.73 (t, J = 7.19 Hz, 2H), 2.88 (t, J = 7.19 Hz, 2H), 2.94 (t, J = 7.19 Hz, 2H), 3.03 | 377 | 375 | |
| | | (t, J = 7.30 Hz, 2H), | | | |
| | | 3.09-3.14 (m, 1H), | | | |
| | | 7.17 (t, J = 7.88 Hz, | | | |
| | | 1H), 7.26 (d, J = | | | |
| | | 8.00 Hz, 1H), 7.31 | | | |
| | | (dd, J = 7.60, 0.80 | | | |
| | | Hz, 1H), 9.66 (brs, | | | |
| | | 1H), 12.17 (brs, 1H) | | | |
| F-186 | | (400 MHz, CDCl3) 0.96 (s, 2H), 1.16- 1.20 (m, 3H), 1.45 (s, 9H), 1.53-1.73 (m, 6H), 1.85 (s, 1H), 2.29 (s, 3H), 2.82 (t, J = 8.05 | 516 | 514 | Racemic form |
| | | Hz, 3H), 2.96-2.99 | | | |
| | | (m, 1H), 3.09 (d, J = | | | |
| | | 6.84 Hz, 2H), 3.16 | | | |
| | | (d, J = 6.84 Hz, 2H), | | | |
| | | 3.92 (s, 2H), 7.08 | | | |
| | | (t, J = 7.94 Hz, 1H), | | | |
| | | 7.16 (d, J = 7.72 | | | |
| | | Hz, 1H), 7.62 (d, J = | | | |
| | | 7.72 Hz, 1H), | | | |
| | | 8.84 (s, 1H) | | | |
| F-187 | | (400 MHz, CDCl3) 0.98-1.01 (m, 2H), 1.21-1.24 (m, 3H), 1.73-1.75 (m, 3H), 2.23 (t, J = 6.29 Hz, 2H), 2.31 (s, 3H), 3.05-3.20 (m, 7H), 3.35 (t, J = 5.51 | 430 | 428 | |
| | | Hz, 2H), 3.75 (t, J = | | | |
| | | 7.06 Hz, 2H), 7.08 (t, | | | |
| | | J = 8.05 Hz, 1H), 7.16 | | | |
| | | (d, J = 7.94 Hz, 1H), | | | |
| | | 7.64 (d, J = 7.94 Hz, | | | |
| | | 1H), 8.73(s, 1H) | | | |

TABLE 178

| | | | | | Infor- |
|---|---|---|---|---|---|
| | | | ms | | mation of |
| Example | Chemical structure | NMR | M + H | M − H | structure |
| F-188 | | (400 MHz, CDCl3) 0.90-0.99 (m, 4H), 1.17-1.28 (m, 6H), 1.67-1.75 (m, 7H), 2.33 (s, 3H), 2.74-2.82 (m, 4H), 2.90-2.93 (m, 1H), 3.09 (t, J = 7.17 | 425 | 423 | |
| | | Hz, 2H), 3.83 (s, | | | |
| | | 3H), 4.35 (d, J = 5.95 | | | |
| | | Hz, 2H), 6.50 (s, | | | |
| | | 1H), 6.67-6.69 (m, | | | |
| | | 2H), 7.08 (d, J = | | | |
| | | 7.50 Hz, 1H) | | | |
| F-189 | | (400 MHz, MeOH- D4) 1.31-1.35 (m, 5H), 1.86-1.91 (m, 6H), 2.26 (s, 3H), 2.75 (t, J = 12.46 Hz, 1H), 2.93 (t, J = 12.79 Hz, 1H), 3.14 (t, J = 6.62 Hz, 4H), 3.35-3.39 (m, | 416 | 414 | Racemic form |
| | | 3H), 3.66 (s, 4H), | | | |
| | | 7.15 (t, J = 7.94 Hz, | | | |
| | | 1H), 7.25 (dd, J = | | | |
| | | 13.67, 7.94 Hz, 2H) | | | |
| F-190 | | (400 MHz, CDCl3) 0.94-1.00 (m, 2H), 1.19-1.23 (m, 3H), 1.42-1.48 (m, 2H), 1.69-1.73 (m, 3H), 2.08 (s, 3H), 2.27 (s, 3H), 2.43 (t, J = 11.58 Hz, 1H), | 458 | 456 | Racemic form |
| | | 2.77-2.82 (m, 3H), | | | |
| | | 2.97-3.19 (m, 6H), | | | |
| | | 3.67-3.72 (m, 1H), | | | |
| | | 4.33 (d, J = 8.82 Hz, | | | |
| | | 1H), 7.08 (t, J = 7.94 | | | |
| | | Hz, 1H), 7.16 (d, J = | | | |
| | | 7.50 Hz, 1H), 7.58 (d, | | | |
| | | J = 7.50 Hz, 1H), 8.95 | | | |
| | | (d, J = 58.88 Hz, 1H) | | | |
| F-191 | | (400 MHz, CDCl3) 0.95-0.98 (m, 4H), 1.17-1.25 (m, 6H), 1.67-1.72 (m, 7H), 2.13 (s, 3H), 2.15 (s, 3H), 2.27 (s, 3H), 2.78-2.82 (m, 2H), 2.98 (s, 1H), 3.28-3.33 (m, 1H), 3.72 (dd, J = 8.38, 4.19 Hz, 1H), 3.92 (s, 1H), 4.19-4.26 (m, 2H), 4.44 (t, J = 9.92 Hz, 1H), 6.95- | 478 | 476 | Racemic form |
| | | 6.97 (m, 2H), 7.74 | | | |
| | | (d, J = 7.94 Hz, | | | |
| | | 1H), 9.07 (s, 1H) | | | |

TABLE 179

| | | | ms | | Information |
|---|---|---|---|---|---|
| Example | Chemical structure | NMR | M + H | M − H | of structure |
| F-192 | | (400 MHz, CDCl3) 0.92 (d, J = 6.45 Hz, 6H), 0.94-0.98 (m, 2H), 1.15-1.20 (m, 2H), 1.57-1.63 (m, 3H), 2.14 (s, 3H), 2.71-2.75 (m, 2H), 2.93-2.99 (m, 1H), 3.08 -3.11 (m, 2H), 3.16-3.19 (m, 2H), | 357 | 355 | |
| | | 6.51 (dd, J = 8.06, | | | |
| | | 2.42 Hz, 1H), 6.93 | | | |
| | | (d, J = 8.46 Hz, 1H), | | | |
| | | 7.27 (d, J = 2.82 Hz, 1H), | | | |
| | | 7.59 (s, 1H), 8.78 (s, 1H) | | | |
| F-193 | | (400 MHz, CDCl3) 0.89-0.94 (m, 8H), 1.13-1.19 (m, 2H), 1.59-1.63 (m, 3H), 2.15 (s, 3H), 2.42-2.54 (m, 2H), 2.74-2.87 (m, 3H), 2.94-3.00 (m, 1H), 3.15 (dd, J = 13.90, 11.48 Hz, 1H), 3.75-3.82 (m, 1H), | 415 | 413 | Racemic form |
| | | 5.04-5.08 (m, 2H), | | | |
| | | 5.71-5.81 (m, 1H), | | | |
| | | 7.04 (dd, J = 8.06, | | | |
| | | 8.06 Hz, 1H), 7.15 | | | |
| | | (d, J = 7.66 Hz, 1H), | | | |
| | | 7.43 (d, J = 8.06 Hz, 1H), | | | |
| | | 8.08 (s, 1H) | | | |
| F-194 | | (400 MHz, CDCl3) 0.88-0.95 (m, 11H), 1.12-1.18 (m, 3H), 1.25-1.34 (m, 1H), 1.37-1.44 (m, 1H), 1.60-1.66 (m, 2H), 1.69-1.76 (m, 2H), 2.14 (s, 3H), 2.76-2.85 (m, 3H), 2.93-2.98 | 417 | 415 | Racemic form |
| | | (m, 1H), 3.13 (dd, | | | |
| | | J = 13.70, 10.88 Hz, | | | |
| | | 1H), 3.66-3.74 (m, 1H), | | | |
| | | 7.04 (dd, J = 8.46, 8.46 | | | |
| | | Hz, 1H), 7.14 (d, J = 8.06 | | | |
| | | Hz, 1H), 7.43 (d, J = 8.06 | | | |
| | | Hz, 1H), 8.23 (s, 1H) | | | |

TABLE 180

| | | | MS | | Information |
|---|---|---|---|---|---|
| Example | Chemical structure | NMR | M + H | M − H | of structure |
| F-195 | | (400 MHz, CDCl3) 0.83-0.90 (m, 1H), 0.94 (d, J = 6.40 Hz, 6H), 1.05-1.18 (m, 3H), 1.48-1.70 (m, 5H), 1.73-1.83 (m, 2H), 2.10 (s, 3H), 2.72-2.80 (m, 2H), 2.83-2.97 (m, 2H), 3.11- 3.20 (m, 1H), 3.31-3.42 (m, 2H), 3.68-3.77 (m, 1H), 4.43 (s, 2H), 7.01 (t, J = 8.05 Hz, 1H), 7.13 (d, J = 7.72 Hz, 1H), 7.25-7.38 (m, 6H), | 523 | 521 | Racemic form |
| | | 8.79 (brs, 1H) | | | |
| F-196 | | (400 MHz, CDCl3) 0.86-0.98 (m, 7H), 1.15-1.23 (m, 3H), 1.49-1.71 (m, 4H), 1.77-1.96 (m, 3H), 2.12 (s, 3H), 2.73- 2.80 (m, 2H), 2.89 (dd, J = 14.22, 4.30 Hz, 1H), 2.96-3.03 (m, 1H), 3.15 (dd, J = 14.00, 10.45 Hz, 1H), 3.60 (t, J = 6.18 | 433 | 431 | Racemic form |
| | | Hz, 2H), 3.74-3.83 (m, | | | |
| | | 1H), 7.03 (t, J = 8.05 Hz, | | | |
| | | 1H), 7.15 (d, J = 7.94 Hz, | | | |
| | | 1H), 7.34 (d, J = 7.72 | | | |
| | | Hz, 1H), 8.80 (brs, 1H) | | | |
| F-197 | | (400 MHz, CDCl3) 0.86-0.94 (m, 7H), 1.09-1.30 (m, 5H), 1.54-1.61 (m, 1H), 1.72-1.81 (m, 2H), 2.16 (s, 3H), 2.44-2.56 (m, 2H), 2.73-2.80 (m, 2H), 2.84 (dd, J = 14.03, 3.59 Hz, 1H), 2.93-3.00 (m, 1H), 3.17 (dd, J = 13.45, | 429 | 427 | Racemic form |
| | | 11.13 Hz, 1H), 3.73-3.81 | | | |
| | | (m, 1H), 5.04-5.11 (m, | | | |
| | | 2H), 5.71-5.82 (m | | | |
| | | 1H), 7.05 (t, J = 7.77 Hz, | | | |
| | | 1H), 7.15 (d, J = 8.12 | | | |
| | | Hz, 1H), 7.46 (d, | | | |
| | | J = 7.65 Hz, 1H), 7.96 | | | |
| | | (brs, 1H) | | | |

TABLE 181

| | | | MS | | Information |
|---|---|---|---|---|---|
| Example | Chemical structure | NMR | M + H | M − H | of structure |
| F-198 | | (400 MHz, CDCl3) 0.84-0.92 (m, 7H), 1.09-1.19 (m, 3H), 1.20-1.28 (m, 2H), 1.52-1.62 (m, 1H), 1.75 (d, J = 38.82 Hz, 2H), 2.06 (s, 3H), 2.24 (s, 3H), 2.47- 2.55 (m, 2H), 2.75 (t, J = 7.72 Hz, 2H), 2.81 (dd, J = 14.00, 3.86 Hz, 1H), 2.93- | 409 | 407 | Racemic form |
| | | 2.99 (m, 1H), 3.11 (dd, | | | |
| | | J = 14.00, 11.14 Hz, 1H), | | | |
| | | 3.75-3.84 (m, 1H), 5.04- | | | |
| | | 5.12 (m, 2H), 5.71- | | | |
| | | 5.82 (m, 1H), 6.90-6.96 | | | |
| | | (m, 2H), 7.43 (d, | | | |
| | | J = 8.60 Hz, 1H), 7.50 | | | |
| | | (brs, 1H) | | | |
| F-199 | | (400 MHz, CDCl3) 0.88 (d, J = 6.62 Hz, 6H), 0.93-0.99 (m, 1H), 1.06-1.13 (m, 1H), 1.17-1.21 (m, 2H), 1.24- 1.29 (m, 2H), 1.52-1.62 (m, 1H), 1.71-1.81 (m, 2H), 1.93-2.13 (m, 2H), 2.17 (s, 3H), 2.76 (dd, J = 7.83, 3.91 Hz, 2H), 2.92 (dd, J = 14.11, 4.41 Hz, 1H), 2.99-3.04 (m, 1H), 3.18 (dd, J = 14.22, 10.26 Hz, 1H), 3.66-3.81 (m, 2H), 3.93-4.01 (m, 1H), 7.06 (t, J = 8.05 Hz, 1H), 7.16 (d, J = 7.50 Hz, 1H), 7.45 (d, J = 7.72 Hz, 1H), 8.23 (brs, 1H) | 433 | 431 | Racemic form |
| F-200 | | (400 MHz, CDCl3) 0.67-0.73 (m, 1H), 0.89 (dd, J = 6.49, 0.70 Hz, 6H), 1.00-1.13 (m, 3H), 1.21-1.30 (m, 2H), 1.54-1.63 (m, 1H), 1.72-1.80 (m, 2H), 2.32 (s, 3H), 2.37-2.49 (m, 2H), 2.57-2.71 (m, 3H), 2.80-2.88 (m, 2H), 3.68-3.75 (m, 1H), 3.81 (s, 3H), 4.17- 4.27 (m, 2H), 4.95-5.02 | 439 | 437 | Racemic form |
| | | (m, 2H), 5.63-5.74 | | | |
| | | (m, 1H), 6.19 (brs, 1H), | | | |
| | | 6.61 (s, 1H), 6.64 (d, | | | |
| | | J = 7.42 Hz, 1H), 6.95 | | | |
| | | (d, J = 7.42 Hz, 1H) | | | |

TABLE 182

| | | | MS | | Information |
|---|---|---|---|---|---|
| Example | Chemical structure | NMR | M + H | M − H | of structure |
| F-201 | | (400 MHz, CDCl3) 0.87 (d, J = 6.62 Hz, 6H), 0.92-0.99 (m, 1H), 1.06-1.11 (m, 1H), 1.12-1.30 (m, 4H), 1.51-1.62 (m, 1H), 1.69-1.79 (m, 2H), 1.93-2.14 (m, 2H), 2.07 (s, 3H), 2.25 (s, 3H), 2.74 (t, J = 7.94 Hz, 2H), 2.87 (dd, | 413 | 411 | Racemic form |
| | | J = 14.11, 4.63 Hz, 1H), | | | |
| | | 2.97-3.05 (m, 1H), 3.12 | | | |
| | | (dd, J = 14.00, 10.03 Hz, | | | |
| | | 1H), 3.66-3.78 (m, 2H), | | | |
| | | 3.94-4.02 (m, 1H), 6.91- | | | |
| | | 6.96 (m, 2H), 7.42 (d, | | | |
| | | J = 8.60 Hz, 1H), 7.75 | | | |
| | | (brs, 1H) | | | |
| F-202 | | (400 MHz, CDCl3) 0.77-0.83 (m, 1H), 0.90 (d, J = 6.62 Hz, 6H), 0.96-1.03 (m, 1H), 1.07-1.15 (m, 2H), 1.23- 1.31 (m, 2H), 1.54- 1.64 (m, 1H), 1.73-1.81 (m, 2H), 1.85-1.93 (m, 1H), 1.98-2.07 (m, 1H), 2.32 (s, 3H), 2.60 (dd, J = 14.22, 5.62 Hz, 1H), 2.63-2.69 (m, 2H), 2.33 (dd, J = 14.11, 9.26 Hz, 1H), 2.88-2.94 (m, 1H), | 443 | 441 | Racemic form |
| | | 3.56-3.67 (m, 2H), 3.82 | | | |
| | | (s, 3H), 3.87-3.94 (m, | | | |
| | | 1H), 4.21-4.30 (m, 2H), | | | |
| | | 6.27 (brt, J = 5.73 Hz, | | | |
| | | 1H), 6.63 (s, 1H), 6.66 | | | |
| | | (d, J = 8.16 Hz, 1H), 6.98 | | | |
| | | (d, J = 7.28 Hz, 1H) | | | |
| F-203 | | (400 MHz, CDCl3) 0.82-0.91 (m, 1H), 0.94-0.97 (m, 2H), 1.17-1.19 (m, 2H), 1.60-1.64 (m, 6H), 1.76-1.85 (m, 2H), 2.25 (s, 3H), 2.28 (s, 3H), 2.76-2.79 (m, 4H), 2.95-2.99 (m, 1H), 3.11-3.15 (m, 4H), 7.07 | 430 | 428 | Racemic form |
| | | (t, J = 8.05 Hz, 1H), 7.16 | | | |
| | | (d, J = 7.72 Hz, 1H), 7.56 | | | |
| | | (s, 1H), | | | |
| | | 9.12 (s, 1H) | | | |

TABLE 183

| | | | MS | | Information |
|---|---|---|---|---|---|
| Example | Chemical structure | NMR | M + H | M − H | of structure |
| F-204 | | (400 MHz, CDCl3) 0.89-1.00 (m, 8H), 1.11-1.20 (m, 2H), 1.62-1.70 (m, 3H), 2.33 (s, 3H), 2.70- 2.80 (m, 2H), 2.80- 2.87 (m, 2H), 2.89- 2.98 (m, 1H), 3.06- 3.12 (m, 2H), 3.81 (s, 3H), 4.28-4,34 (m, 2H), 6.62-6.71 (m, 2H), 7.08 (brs, 1H) | 419 | 417 | |
| F-205 | | (400 MHz, CDCl3) 0.86-0.94 (m, 2H), 0.94-1.00 (m, 6H), 1.11-1.19 (m, 2H), 1.64-1.72 (m, 3H), 2.16 (s, 3H), 2.23 (s, 3H), 2.72- 2.82 (m, 4H), 2.88-2.97 (m, 1H), 3.06-3.13 (m, 2H), 3.81 (s, 3H), 4.31-4.36 (m, 2H), 6.38 (brs, 1H), 6.64 (brs, 1H), 6.98 (brs, 1H) | 399 | 397 | |
| F-206 | | (400 MHz, CDCl3) 1.04-1.10 (m, 2H), 1.16-1.23 (m, 2H), 2.13 (s, 3H), 2.24 (s, 3H), 2.27 (s, 3H), 3.03- 3.14 (m, 3H), 3.15- 3.23 (m, 2H), 4.48 (s, 2H), 6.66 (d, J = 7.94 Hz, 1H), 6.91 (s, | 438 | 436 | |
| | | 1H), 6.94 (d, J = 7.94 | | | |
| | | Hz, 1H), 7.07 (dd, | | | |
| | | J = 8.05, 4.02 Hz, 1H), | | | |
| | | 7.16 (d, J = 7.72 Hz, | | | |
| | | 1H), 7.59 (d, J = 7.94 | | | |
| | | Hz, 1H), 8.62 (brs, 1H) | | | |
| F-207 | | (400 MHz, CDCl3) 1.04-1.10 (m, 2H), 1.17-1.24 (m, 2H), 2.16 (s, 3H), 2.20 (s, 3H), 2.29 (s, 3H), 3.04-3.13 (m, 3H), 3.15-3.22 (m, 2H), 4.46 (s, 2H), 6.50 (dd, J = 8.16, 2.21 Hz, 1H), 6.55 (d, | 438 | 436 | |
| | | J = 2.21 Hz, 1H), 6.96 | | | |
| | | (d, J = 8.16 Hz, 1H), | | | |
| | | 7.08 (t, J = 8.05 Hz, | | | |
| | | 1H), 7.16 (d, J = 7.94 | | | |
| | | Hz, 1H), 7.62 (d, | | | |
| | | J = 7.94 Hz, 1H), | | | |
| | | 8.56 (brs, 1H) | | | |

TABLE 184

| | | | MS | | information |
|---|---|---|---|---|---|
| Example | Chemical structure | NMR | M + H | M − H | of structure |
| F-208 | | (400 MHz, CDCl3) 0.91-0.95 (m, 2H), 0.96 (d, J = 6.40 Hz, 6H), 1.14-1.20 (m, 2H), 1.63-1.71 (m, 3H), 2.74-2.80 (m, 2H), 2.86 (t, J = 6.95 Hz, 2H), 2.94 (tt, J = 7.12, 3.96 Hz, 1H), 3.10 (t, J = 6.95 Hz, 2H), 3.82 (s, 3H), 4.36 (d, J = 6.18 Hz, 2H), 6.74 (dd, J = 8.93, 4.30 Hz, | 389 | 387 | |
| | | 1H), 6.77 (brs, 1H), | | | |
| | | 6.81-6.91 (m, 2H) | | | |
| F-209 | | (400 MHz, CDCl3) 1.10-1.15 (m, 2H), 1.22-1.31 (m, 2H), 2.20 (s, 3H), 3.04 (t, J = 5.95 Hz, 2H), 3.15- 3.21 (m, 3H), 5.05 (s, 2H), 7.06 (t, J = 7.94 Hz, 1H), 7.15 (d, J = 7.94 Hz, 1H), 7.56 (d, J = | 464 | 462 | |
| | | 8.60 Hz, 1H), 7.74 | | | |
| | | (dd, J = 5.51, | | | |
| | | 3.09 Hz, 2H), 7.88 | | | |
| | | (dd, J = 5.18, | | | |
| | | 2.98 Hz, 2H), | | | |
| | | 8.46 (s, 1H) | | | |
| F-210 | | (400 MHz, CDCl3) 0.95 (dd, J = 9.70, 7.06 Hz, 2H), 1.14- 1.22 (m, 2H), 1.24 (dd, J = 12.90, 6.29 Hz, 6H), 1.97-2.01 (m, 2H), 2.28 (s, 3H), 2.73-2.77 (m, 2H), 2.96-3.02 (m, 1H), 3.09 (t, J = 6.29 Hz, | 433 | 431 | |
| | | 2H), 3.16 (t, J = 6.18 | | | |
| | | Hz, 2H), 3.66 (s, 3H), | | | |
| | | 7.05 (dd, J = 23.27, | | | |
| | | 15.11 Hz, 1H), | | | |
| | | 7.17 (t, J = 8.38 Hz, | | | |
| | | 1H), 7.56 (t, J = | | | |
| | | 12.68 Hz, 1H), | | | |
| | | 8.96 (s, 1H) | | | |
| F-211 | | (400 MHz, CDCl3) 0.86-0.99 (m, 2H), 1.10-1.37 (m, 4H), 1.61- 1.79 (m, 7H), 1.90-2.00 (m, 1H), 2.16 (s, 3H), 2.18- 2.32 (m, 1H), 2.26 (s, 3H), 2.71-2.79 (m, 2H), 2.97-3.15 (m, 4H), 3.57-3.67 (m, 1H), 6.93-7.00 (m, | 431 | 429 | Racemic form |
| | | 2H), 7.54 (d, J = | | | |
| | | 8.82 Hz, 1H), 7.84 | | | |
| | | (brs, 1H) | | | |

TABLE 185

| | | | Ms | | Information |
|---|---|---|---|---|---|
| Example | Chemical structure | NMR | M + H | M − H | of structure |
| F-212 | | (400 MHz, CDCl3) 0.84-0.94 (m, 7H), 1.13-1.31 (m, 5H), 1.48- 1.94 (m, 7H), 2.12 (s, 3H), 2.71- 2.78 (m, 2H), 2.88 (dd, J = 14.11, 4.41 Hz, 1H), 2.96- 3.02 (m, 1H), 3.16 (dd, J = 14.11, 10.59 Hz, 1H), 3.60 (t, J = 6.18 Hz, | 447 | 445 | Racemic form |
| | | 2H), 3.74-3.82 | | | |
| | | (m, 1H), 7.03 (t, | | | |
| | | J = 8.05 Hz, 1H), | | | |
| | | 7.15 (d, J = 7.72 Hz, | | | |
| | | 1H), 7.35 (d, J = | | | |
| | | 7.72 Hz, 1H), 8.71 | | | |
| | | (s, 1H) | | | |
| F-213 | | (400 MHz, DMSO- D6) 0.85 (d, J = 6.72 Hz, 6H), 1.03-1.10 (m, 4H), 1.17-1.25 (m, 2H), 1,60-1.59 (m, 1H), 1.62- 1.72 (m, 2H), 2.06 (s, 3H), 2,54-2.92 (m, 6H), 3.08-3.12 (m, 1H), 3.90- 4.00 (m, 1H), 7.11-7.27 (m, 3H) 9.63 (s, 1H) | 447 | 445 | Racemic form |
| F-214 | | (400 MHz, CDCl3) 0.83-0.91 (m, 7H), 1.14-1.20 (m, 3H), 1.22-1.28 (m, 2H), 1.49-1.96 (m, 7H), 2.03 (s, 3H), 2.23 (s, 3H), 2.69-2.77 (m, 2H), 2.82 (dd, J = 14.00, 4.74 Hz, 1H), 2.94-3.02 (m, 1H), 3.09 (dd, J = 14.11, 10.59 Hz, 1H), 3.61 (t, J = 6.18 Hz, 2H), 3.74-3.84 (m, 1H), 6.87- | 427 | 425 | Racemic form |
| | | 8.95 (m, 2H), 7.31 (d, | | | |
| | | J = 7.94 Hz, 1H), | | | |
| | | 8.17 (brs, 1H) | | | |
| F-215 | | (400 MHz, CDCl3) 0.89 (d, J = 6.62 Hz, 6H), 0.93-0.98 (m, 2H), 1.15-1.21 (m, 2H), 1.25-1.32 (m, 2H), 1.53- 1.64 (m, 1H), 1.74-1.83 (m, 2H), 2.19 (s, 3H), 2.26 (s, 3H), 2.73-279 (m, 2H), 2.96 (tt, J = 7.12, 3.96 Hz, 1H), 3.02-3.00 (m, | 369 | 367 | |
| | | 2H), 3.14-3.19 (m, 2H), | | | |
| | | 6.93-6.98 (m, 2H), 7.62 | | | |
| | | (d, J = 8.82 Hz, 1H), 8.28 | | | |
| | | (brs, 1H) | | | |

TABLE 186

| | | | Ms | | Information |
|---|---|---|---|---|---|
| Example | Chemical structure | NMR | M + H | M − H | of structure |
| F-216 | | (400 MHz, CDCl3) 0.89 (d, J = 6.62 Hz, 6H), 0.93-0.99 (m, 2H), 1.15-1.22 (m, 2H), 1.24-1.33 (m, 2H), 1.53-1.65 (m, 1H), 1.74-1.84 (m, 2H), 2.11 (s, 3H), 2.26 (s, 3H), 2.77 (t, J = 7.83 Hz, 2H), 2.96 (tt, J = 7.12, 3,96 | 369 | 367 | |
| | | Hz, 1H), 3.07 (t, J = 6.40 | | | |
| | | Hz, 2H), 3.18 (t, J = | | | |
| | | 6.29 Hz, 2H), 6.96 (d, | | | |
| | | J = 7.50 Hz, 1H), 7.06 | | | |
| | | (dd, J = 7.94, 7.50 Hz, | | | |
| | | 1H), 7.47 (d, J = | | | |
| | | 7.94 Hz, 1H), 8.33 | | | |
| | | (brs, 1H) | | | |
| F-217 | | (400 MHz. CDCl3) 0.90 (d, J = 6.62 Hz, 6H), 0.90-0.94 (m, 2H), 1.12-1.19 (m, 2H), 1.25-1.33 (m, 2H), 1.54-165 (m, 1H), 1.74-1.84 (m, 2H), 2.33 (s, 3H), 2.73 (t, J = 7.83 Hz, 2H), 2.80 (t, J = 7.28 Hz, 2H), 292 (tt, J = 7.12, 3.96 Hz, 1H), 3.09 | 399 | 397 | |
| | | (t, J = 7.28 Hz, 2H), | | | |
| | | 3.83 (s, 3H), 4.35 (d, | | | |
| | | J = 5.95 Hz, 2H), 6.43 | | | |
| | | (brs, 1H), 6.67 (s, 1H), | | | |
| | | 6.69 (d, J = 7.50 Hz, | | | |
| | | 1H), 7.08 (d, J = 7.50 | | | |
| | | Hz, 1H) | | | |
| F-218 | | (400 MHz, CDCl3) 0.99-1.03 (m, 2H), 1.12 (t, J = 7.77 Hz, 6H), 1.19 (dd, J = 12.75, 7.19 Hz, 2H), 2.28 (s, 3H), 2.98-3.18 (m, 7H), 3.53-3.59 (m, 1H), 3.81 (t, J = 6.96 Hz, 2H), 7.08 (t, J = 8.00 | 391 | 389 | |
| | | Hz, 1H), 7.16 (d, | | | |
| | | J = 7.65 Hz, 1H), | | | |
| | | 7.60 (d, J = 7.65 | | | |
| | | Hz, 1H), 9.03 (s, 1H) | | | |

TABLE 187

| | | | Ms | | Information |
|---|---|---|---|---|---|
| Example | Chemical structure | NMR | M + H | M − H | of structure |
| F-219 | | (400 MHz, CDCl3) 0.89 (s, 9H), 0.94- 1.02 (m, 2H), 1.19-1.32 (m, 4H), 1.73-1.81 (m, 2H), 2.14 (s, 3H), 2.16 (d, J = 3.75 Hz, 3H), 2.25 (s, 3H), 2.77 (tt, J = 11.03, 4.70 Hz, 2H), 2.96- 3.01 (m, 1H), 3.30 (t, J = 11.03 Hz, 1H), 3.72 (dt, J = 18.01, 5.24 Hz, 1H), 3.91 (dt, J = 15.36, 4.80 Hz, 1H), 4.22 (ddd, J = | 466 | 464 | Racemic form |
| | | 12.52, 8.10, 4.80 Hz, 2H), | | | |
| | | 4.45 (dd, J = 11.47, 8.38 Hz, | | | |
| | | 1H), 6.95 (d, J = 10.14 Hz, | | | |
| | | 2H), 7.74 (d, J = 7.94 Hz, | | | |
| | | 1H), 9.03 (s, 1H) | | | |
| F-220 | | (400 MHz, CDCl3) 0.89 (s, 3H), 0.90 (s, 3H), 0.97 (td, J = 9.92, 4.78 Hz, 2H), 1.22-1.32 (m, 3H), 1.56-1.63 (m, 2H), 1.76-1.84 (m, 2H), 2.14 (s, 3H), 2.16 (s, 3H), 2.25 (s, 3H), 2.78 (dq, J = 30.77, 7.54 Hz, 2H), 2.96-3.01 (m, 1H), 3.30 (t, J = 10.92 Hz, 1H), 3.71 (dd, J = 19.08, 10.70 Hz, 1H), 3.88-3.95 (m, 1H), 4.16-4.26 (m, | 452 | 450 | Racemic form |
| | | 2H), 4.45 (dd, J = 11.47, | | | |
| | | 8.38 Hz, 1H), 6.95 (d, | | | |
| | | J = 11.25 Hz, 2H), 7.76 | | | |
| | | (d, J = 8.16 Hz, 1H), | | | |
| | | 9.03 (s, 1H) | | | |
| F-221 | | (400 MHz, CDCl3) 1.00-1.04 (m, 2H), 1.17-1.35 (m, 8H), 2.29 (s, 3H), 2.42-2.49 (m, 1H), 3.00-3.09 (m, 3H), 3.20 (t, J = 6.29 Hz, 2H), 4.62 (d, J = 5.07 Hz, 2H), 6.61 (brs, 1H), 7.09 (t, J = 8.05 Hz, 1H), 7.17 (d, J = 7.50 | 404 | 402 | |
| | | Hz, 1H), 7.63 (d, | | | |
| | | J = 7.72 Hz, 1H), 8.31 | | | |
| | | (brs, 1H) | | | |

TABLE 188

| | | | Ms | | Information |
|---|---|---|---|---|---|
| Example | Chemical structure | NMR | M + H | M − H | of structure |
| F-222 | | (400 MHz, DMSO-D6) 1.09-1.10 (m, 13H), 1.96-1.99 (m, 3H), 2.04-2.05 (m, 3H), 2.22 (s, 3H), 2.92 (t, J = 6.26 Hz, 2H), 3.08-3.15 (m, 1H), 3.24- 4.13 (m, 8H), 6.93 (d, J = 8.12 Hz, 1H), 6.98 (s, 1H), 7.20 (dd, J = 16.46, 8.12Hz, 1H), 9.52 (d, J = 12.06 Hz, 1H) | 468 | 466 | Racemic form |
| F-223 | | (400 MHz, DMSO-D6) 0.93-1.14 (m, 13H), 1.56-1.61 (m, 2H), 1.96-1.99 (m, 3H), 2.03-2.04 (m, 3H), 2.22 (s, 3H), 2.69- 2.74 (m, 2H), 3.08- 3.14 (m, 1H), 3.19-4.12 (m, 6H), 6.93 (d, J = 7.88 Hz, 1H), 6.99 (s, 1H), 7.19 (dd, J = 14.15, 7.88 Hz, 1H), 9.53 (d, J = 10.44 Hz, 1H) | 452 | 450 | Racemic form |
| F-224 | | (400 MHz, CDCl3) 0.85-0.92 (m, 7H), 1.01-1.12 (m, 3H), 1.24- 1.30 (m, 3H), 1.54- 1.62 (m, 2H), 1.70- 1.79 (m, 2H), 2.14- 2.26 (m, 2H), 2.21 (s, 3H), 2.66-2.79 (m, 2H), 2.95-3.07 (m, 2H), 3.26-3.35 (m, 1H), 3.68-3.76 (m, 2H), 3.82-3.90 (m, 1H), 7.05 (t, J = 8.12 Hz, 1H), 7.14 (d, J = 8.35 Hz, 1H), 7.51 (d, J = 8.58 Hz, 1H), 7.72 (dd, J = 5.45, 2.90 | 562 | 560 | Racemic form |
| | | Hz, 2H), 7.83 (dd, | | | |
| | | J = 5.45, 3.13 Hz, | | | |
| | | 2H), 8.22 (brs, 1H) | | | |

TABLE 189

| | | | Ms | | Information |
|---|---|---|---|---|---|
| Example | Chemical structure | NMR | M + H | M − H | of structure |
| F-225 | | (400 MHz, CDCl3) 0.85-0.97 (m, 7H), 1.13-1.20 (m, 3H), 1.24-1.31 (m, 2H), 1.52-1.63 (m, 1H), 1.71-1.94 (m, 4H), 2.13 (s, 3H), 2.68-2.80 (m, 4H), 2.88 (dd, J = 14.00, 4.08 Hz, 1H), 2.96-3.04 (m, 1H), 3.16 (dd, J = 13.89, 10.59 Hz, | 432 | 430 | Racemic form |
| | | 1H), 3.80-3.89 (m, 1H), | | | |
| | | 7.04 (t, J = 7.94 Hz, 1H), | | | |
| | | 7.15 (d, J = 7.72 Hz, | | | |
| | | 1H), 7.38 (d, J = 7.94 Hz, | | | |
| | | 1H), 8.69 (brs, 1H) | | | |
| F-226 | | (400 MHz, DMSO- D6) 0.99 (t, J = 9.15 Hz, 2H), 1.03-1.11 (m, 2H), 1.19 (d, J = 15.66 Hz, 6H), 1.87 (t, J = 8.60 Hz, 2H), 2.19 (s, 3H), 2.69 (dd, J = 10.26, 6.73 Hz, 2H), 2.88 (t, J = 6.95 Hz, 2H), 3.03 (t, J = 7.17 Hz, 2H), 3.11- | 419 | 417 | |
| | | 3.13 (m, 1H), 7.17 (t, J = | | | |
| | | 7.94 Hz, 1H), 7.26 | | | |
| | | (d, J = 7.72 Hz, 1H), | | | |
| | | 7.31 (d, J = 7.94 Hz, 1H), | | | |
| | | 9.66 (s, 1H), 12.24 | | | |
| | | (s, 1H) | | | |
| F-227 | | (400 MHz, CDCl3) 0.86-0.95 (m, 4H), 1.06-1.43 (m, 9H), 1.58-1.65 (m, 4H), 2.04 (s, 3H), 2.05 (s, 3H), 2.28 (s, 3H), 2.35- 2.47 (m, 1H), 2.58-2.63 (m, 1H), 2.77 (t, J = 8.35 Hz, 2H), 3.32- 3.37 (m, 1H), 3.79-3.95 (m, 3H), 5.57 (d, J = 7.65 Hz, 1H), 6.98- 7.00 (m, 2H), 7.47 (d, J = 7.65 Hz, 1H), 7.54 | 478 | 476 | |
| | | (s, 1H) | | | |

TABLE 190

| | | | Ms | | Information |
|---|---|---|---|---|---|
| Example | Chemical structure | NMR | M + H | M − H | of structure |
| F-228 | | (400 MHz, CDCl3) 0.64-0.69 (m, 1H), 0.81-1.37 (m, 9H), 1.57- 1.91 (m, 6H), 1.99 (s, 3H), 2.23- 2.42 (m, 5H), 2.68- 2.74 (m, 2H), 3.11- 3.15 (m, 1H) (3.38- 3.46 (m, 1H), 3.69- 3.86 (m, 2H), 3,77 (s, 3H), 4.24 (dd, J = 14.26, 5.22 Hz, 1H), 4.38 (dd, J = | 508 | 506 | |
| | | 14.15, 6.49 Hz, | | | |
| | | 1H), 5.36 (d, J = | | | |
| | | 7.88 Hz, 1H), 6.21 | | | |
| | | (t, J = 5.80 Hz, | | | |
| | | 1H), 6.63 (s, 1H), | | | |
| | | 6.69 (d, J = 7.65 | | | |
| | | Hz, 1H), 7.03 (d, | | | |
| | | J = 7.42 Hz, 1H) | | | |
| F-229 | | (400 MHz, CDCl3) 0.89-0.93 (m, 2H), 0.94-0.98 (m, 6H), 1.11- 1.19 (m, 2H), 1.62-1.71 (m, 3H), 2.72-2.80 (m, 2H), 2.80-2.85 (m, 2H), 2.88-2.96 (m, 1H), 3.05- 3.11 (m, 2H), 3.83 (s, 3H), 4.31- | 389 | 387 | |
| | | 4.35 (m, 2H), 6.53- | | | |
| | | 6.60 (m, 2H), | | | |
| | | 6.64 (brs, 1H), | | | |
| | | 7.11-7.18 (m, 1H) | | | |
| F-230 | | (400 MHz, CDCl3) 0.95 (dt, J = 6.69, 2.37 Hz, 2H), 1.15- 1.23 (m, 8H), 1.61- 1.84 (m, H), 2.28 (d, J = 3.75 Hz, 3H), 2.78 (q, J = 7.42 Hz, 2H), 2.94- 3.02 (m, 1H), 3.07 (t, J = 6.06 Hz, 2H), 3.17 | 447 | 445 | |
| | | (dd, J = 7.17, 4.96 | | | |
| | | Hz, 2H), 3.65 (dd, | | | |
| | | J = 22.27, 6.18 Hz, | | | |
| | | 3H), 7.05 (dd, | | | |
| | | J = 24.37, 16.43 | | | |
| | | Hz, 1H), 7.16 (d, | | | |
| | | J = 7.72 Hz, 1H), | | | |
| | | 7.63 (d, J = 8.16 Hz | | | |
| | | 1H), 8.76 (s, 1H) | | | |

TABLE 191

| | | | Ms | | Information |
|---|---|---|---|---|---|
| Example | Chemical structure | NMR | M + H | M − H | of structure |
| F-231 | | (400 MHz, CDCl3) 0.94 (dt, J = 7.65, 3.91 Hz, 2H), 1.17- 1.27 (m, 8H), 1.99 (dd, J = 9.15, 7.17 Hz, 2H), 2.28 (d, J = 10.37 Hz, 3H), 2.72- 2.78 (m, 5H), 3.04 (ddd, J = 15.82, 8.66, 4.36 Hz, 3H), | 432 | 430 | |
| | | 3.15 (dd, J = 15.11, | | | |
| | | 8.71 Hz, | | | |
| | | 2H), 5.76 (d, J = | | | |
| | | 3.75 Hz, 1H), 7.08 | | | |
| | | (t, J = 8.05 Hz, 1H), | | | |
| | | 7.16 (d, J = 7.94 Hz, | | | |
| | | 1H), 7.63 (d, J = 7.94 | | | |
| | | Hz, 1H), 8.87 (s, 1H) | | | |
| F-232 | | (400 MHz, DMSO- D6) 0.97 (t, J = 3.86 Hz, 2H), 1.07-1.10 (m, 8H), 1.54 (t, J = 7.94 Hz, 2H), 1.60-1.68 (m, 2H), 2.19 (s, 3H), 2.71 (t, J = 7.50 Hz, 2H), 2.88 (t, J = 7.06 Hz, 2H), 3.02- 3.10 (m, 3H), | 433 | 431 | |
| | | 7.17 (t, J = 8.05 Hz, | | | |
| | | 1H), 7.26 (d, J = | | | |
| | | 7.28 Hz, 1H), 7.31 | | | |
| | | (d, J = 7.94 Hz, | | | |
| | | 1H), 9.65 (s, 1H), | | | |
| | | 12.07 (s, 1H) | | | |
| F-233 | | (400 MHz, CDCl3) 1.00-1.02 (m, 2H), 1.17 (d, J = 6.72 Hz, 1H), 1.22- 1.24 (m, 7H), 2.13 (s, 3H), 2.28 (s, 3H), 3.02-3.11 (m, 5H), 3.19 (d, J = 6.72 Hz, 2H), 3.58-3.60 (m, | 432 | 430 | |
| | | 2H), 4.00-4.07 (m, | | | |
| | | 1H), 7.08 (t, | | | |
| | | J = 8.00 Hz, 1H), | | | |
| | | 7.16 (d, J = 7.65 Hz, | | | |
| | | 1H), 7.57 (d, J = | | | |
| | | 8.12 Hz, 1H), 8.89 | | | |
| | | (s, 1H) | | | |
| F-234 | | (400 MHz, CDCl3) 0.89-0.90 (m, 2H), 1.18 (t, J = 6.18 Hz, 2H), 1.45 (s, 9H), 2.29 (s, 3H), 2.93-2.97 (m, 1H), 3.08 (s, 2H), 3.17 (t, J = 6.40 Hz, 2H), 3.94 (q, J = 7.65 Hz, | 460 | 458 | |
| | | 1H), 4.27-4.29 (m, | | | |
| | | 4H), 7.09 (t, J = | | | |
| | | 8.05 Hz, 1H), | | | |
| | | 7.17 (d, J = 7.94 | | | |
| | | Hz, 1H), 7.61 (d. J = | | | |
| | | 8.16 Hz, 1H), 8.47 | | | |
| | | (s, 1H) | | | |

TABLE 192

| | | | Ms | | Information |
|---|---|---|---|---|---|
| Example | Chemical structure | NMR | M + H | M − H | of structure |
| F-235 | | (400 MHz, CDCl3) 0.89 (t, J = 7.28 Hz, 3H), 0.94 (d, J = 6.62 Hz, 3H), 0.95-1.00 (m, 2H), 1.14-1.29 (m, 3H), 1.34-1.48 (m, 2H), 1.53-1.64 (m, 1H), 1.75-1.86 (m, 1H), 2.29 (s, 3H), 2.71- 2.88 (m, 2H), | 389 | 387 | Racemic form |
| | | 2.97 (tt, J = 7.12, | | | |
| | | 3.96 Hz, 1H), 3.04- | | | |
| | | 3.12 (m, 2H), 3.13- | | | |
| | | 3.21 (m, 2H), 7.08 (dd, | | | |
| | | J = 7.94, 7.72 Hz, 1H), | | | |
| | | 7.15 (d, J = 7.94 Hz, | | | |
| | | 1H), 7.64 (d, J = 7.72 | | | |
| | | Hz, 1H), 8.83 | | | |
| | | (brs, 1H) | | | |
| F-236 | | (400 MHz, CDCl3) 0.89 (t, J = 7.28 Hz, 3H), 0.94 (d, J = 6.40 Hz, 3H), 0.94-0.99 (m, 2H), 1.14-1.28 (m, 3H), 1.34-1.48 (m, 2H), 1.52- 1.63 (m, 1H), 1.75-1.85 (m, 1H), 2.18 (s, 3H), 2.26 (s, 3H), 2.70-2.87 (m, 2H), 2.97 (tt, J = 7.12, 3.96 Hz, 1H), 3.05 (t, J = 6.46 Hz, 2H), 3.17 (t, J = 6.46 Hz, 2H), 6.93-6.98 (m, 2H), 7.61 (d, J = 8.60 Hz, | 369 | 367 | Racemic form |
| | | 1H), 8.28 (brs, 1H) | | | |
| F-237 | | (400 MHz, CDCl3) 0.93-0.97 (m, 2H), 1.14-1.22 (m, 8H), 1.62-1.68 (m, 2H), 1.82 (dq, J = 15.99, 4.48 Hz, 2H), 2.29 (s, 3H), 2.78 (dt, J = 15.29, 5.90 Hz, 5H), 2.94-2.99 (m, 1H), 3.07 (t, | 446 | 444 | |
| | | J = 5.95 Hz, 2H), | | | |
| | | 3.17 (t, J = 6.06 | | | |
| | | Hz, 2H), 6.57 (s, | | | |
| | | 1H), 7.08 (t, J = | | | |
| | | 7.94 Hz, 1H), 7.17 | | | |
| | | (t, J = 7.83 Hz, | | | |
| | | 1H), 7.62 (d, | | | |
| | | J = 7.94 Hz, 1H), | | | |
| | | 8.60 (s, 1H) | | | |

TABLE 193

| | | | Ms | | Information |
|---|---|---|---|---|---|
| Example | Chemical structure | NMR | M + H | M − H | of structure |
| F-238 | | (400 MHz, CDCl3) 0.94-0.98 (m, 2H), 1.17-1.23 (m, 2H), 1.26 (dd, J = 9.48, 4.85 Hz, 6H), 1.67- 1.81 (m, 4H), 2.29 (s, 3H), 2.80 (t, J = 7.06 Hz, 2H), 2.95-3.01 (m, 7H), 3.04-3.09 (m, 2H), | 460 | 458 | |
| | | 3.17 (t, J = 6.18 Hz, | | | |
| | | 2H), 7.05 (dd, J = | | | |
| | | 23.38, 15.44 Hz, | | | |
| | | 1H), 7.16 (d, J = | | | |
| | | 7.94 Hz, 1H), | | | |
| | | 7.53-7.62 (m, 1H), | | | |
| | | 8.82 (s, 1H) | | | |
| F-239 | | (400 MHz, CDCl3) 0.94-0.96 (m, 2H), 1.19-1.21 (m, 2H), 1.36 (d, J = 2.65 Hz, 6H), 1.55-1.61 (m, 2H), 2.12-2.18 (m, 2H), 2.31 (d, J = 2.21 Hz, 3H), 2.80 (dd, J = 15.00, 9.26 Hz, 2H), 3.01 (d, J = 14.56 Hz, 7H), 3.16 (d, J = 2.87 Hz, | 446 | 444 | |
| | | 2H), 7.09 (t, J = | | | |
| | | 7.39 Hz, 1H), 7.16 | | | |
| | | (d, J = 7.94 Hz, 1H), | | | |
| | | 7.66 (d, J = 6.84 Hz, | | | |
| | | 1H), 8.72 (s, 1H) | | | |
| F-240 | | (400 MHz, CDCl3) 0.88-1.02 (m, 3H), 1.20-1.28 (m, 7H), 1.37-1.47 (m, 2H), 1.64 (tt, J = 16.43, 5.99 Hz, 2H), 1.81- 1.86 (m, 2H) 1.98 (dt, J = 11.76, 2.92 Hz, 2H), 2.21 (ddd, J = | 487 | 485 | |
| | | 16.21, 8.27, 3.97 | | | |
| | | Hz, 1H), 2.26 (s, 3H), | | | |
| | | 2.77 (dq, J = 8.05, | | | |
| | | 2.46 Hz, 2H), | | | |
| | | 2.99 (ddd, J = 17.15, | | | |
| | | 9.76, 6.23 Hz, 1H), | | | |
| | | 3.12-3.18 (m, 4H), | | | |
| | | 4.14 (ddt, J = 23.80, | | | |
| | | 13.67, 4.00 Hz, 2H), | | | |
| | | 7.05 (dd, J = 21.17, | | | |
| | | 13.23 Hz, 1H), 7.16 | | | |
| | | (d, J = 7.94 Hz, | | | |
| | | 1H), 7,53 (d, J = | | | |
| | | 7.72 Hz, 1H), | | | |
| | | 9.20 (s, 1H) | | | |

TABLE 194

| | | | Ms | | Information |
|---|---|---|---|---|---|
| Example | Chemical structure | NMR | M + H | M − H | of structure |
| F-241 | | (400 MHz, CDCl3) 0.88-1.03 (m, 9H), 1.13-1.30 (m, 6H), 1.70- 1.77 (m, 4H), 2.34 (m, 3H), 2.76- 2.32 (m, 4H), 2.90- 2.95 (m, 1H), 3.10 (t, J = 7.07 Hz, 2H), 3.84 (s, 3H), 4.36 (d, J = 6.03 Hz, 2H), 6.44- | 439 | 437 | |
| | | 6.48 (brm, 1H), 6.67- | | | |
| | | 6.73 (m, 2H), 7.09 (d, | | | |
| | | J = 7.42 Hz, 1H) | | | |
| F-242 | | (400 MHz, CDCl3) 0.95-0.99 (m, 2H), 1.15-1.27 (m, 3H), 1.34- 1.46 (m, 4H), 1.62- 1.56 (m, 2H), 1.69-1.75 (m, 2H), 1.89 (d, J = 11.36 Hz, 2H), 2.30 (s, 3H), 2.82 | 445 | 443 | |
| | | (t, J = 8.12 Hz, 2H), | | | |
| | | 2-94-3.00 (m, 1H), | | | |
| | | 3.06-3.11 (m, 2H), | | | |
| | | 3.15-3.20 (m, 2H), | | | |
| | | 3.31 (s, 3H), 3.41- | | | |
| | | 3.45 (brn, 1H), 7.09 | | | |
| | | (t, J = 8.12 Hz, | | | |
| | | 1H), 7.17 (d, | | | |
| | | J = 7.65 Hz, 1H), | | | |
| | | 7.65 (d, J = 7.88 Hz, | | | |
| | | 1H), 8.76 (brs, 1H) | | | |
| F-243 | | (400 MHz, DMSO- D6) 0.96 (q, J = 11.76 Hz, 4H), 1.09 (t, J = 7.06 Hz, 2H), 1.24-1.33 (m, 3H), 1.59 (dd, J = 15.44, 7.06 Hz, 2H), 1.85 (dd, J = 30.66, 12.13 Hz, 4H), 2.12 (dd, J = 13.89, 10.37 | 459 | 457 | |
| | | Hz, 1H), 2.18 (s, 3H), | | | |
| | | 2.74 (t, J = 7.94 Hz, | | | |
| | | 2H), 2.88 (t, J = 6.84 | | | |
| | | Hz, 2H), 3.03 (t, J = | | | |
| | | 7.17 Hz, 2H), 3.11 | | | |
| | | (t, J = 3.53 Hz, 1H), | | | |
| | | 7.17 (t, J = 7.94 | | | |
| | | Hz, 1H), 7.26 (d, | | | |
| | | J = 7.50 Hz, 1H), | | | |
| | | 7.30 (d, J = 8.16 Hz, | | | |
| | | 1H), 9.66 (s, 1H), | | | |
| | | 12.00 (s, 1H) | | | |

TABLE 195

| | | | | | Infor- |
|---|---|---|---|---|---|
| | | | | | mation |
| Exam- | | | MS | | of |
| ple | Chemical structure | NMR | M + H | M − H | structure |
| F-244 | | (400 MHz, CDCl3) 0.88-0.98 (m, 3H), 1.10-1.33 (m, 8H), 1.61- 1.79 (m, 6H), 2.15 (s, 3H), 2.43- 2.56 (m, 2H), 2.72-2 85 (m, 3H), 2.93- 2.99 (m, 1H), 3.16 (dd, J = 1370, 11.28 Hz, 1H), 3.75-3.82 (m, 1H), 5.07 (d, J = 12.09 Hz, 2H), 5.71-5.81 (m, 1H), 7.04 (dd, J = 8 06, 8 06 Hz, 1H), 7.15 (d, J = 7.66 Hz, 1H), 7.43 (d, J = 7.66 Hz, 1H), 8.03 (s, 1H) | 455 | 453 | Racemic form |
| F-245 | | (400 MHz, CDCl3) 0.86-0.97 (m, 3H), 1.11-1.32 (m, 8H), 1.59- 1.76 (m, 6H), 2.05 (s, 3H), 2.24 (s, 3H), 2.44-2.57 (m, 2H), 2.75- 2.83 (m, 3H), 2.93-2.98 (m, 1H), 3.11 (dd, J = 13.70, 11.28 Hz, 1H), 3.77-3.84, (m, 1H), 5.05-5.09 (m, 2H), 5.71-5.81 (m, 1H), 6.93- 6.91 (m, 2H), 7.40 (d, J = 8.46 Hz, 1H), 7.61 (s, 1H) | 435 | 433 | Racemic form |
| F-246 | | (400 MHz, CDCl3) 0.68-0.73 (m, 1H), 0.90-1.34 (m, 10H), 1.62- 1.79 (m, 6H), 2.32 (s, 3H), 2.38- 2.47 (m, 2H), 2.59 (dd, J = 14.10, 4.43 Hz, 1H), 2.64-2.74 (m, 2H), 2.80 -2.88 (m, 2H), 3.68-3.75 (m, 1H), 3.81 (s, 3H), 4.17-4.26 (m, 2H), 4.97-5.01 (m, 2H), 5.63- 5.73 (m, 1H), 6.22 (t, J = 6.04 Hz, 1H), 6.65-6.61 (m, 2H), 6.94 (d, J = 7.66 Hz, 1H) | 465 | 463 | Racemic form |

TABLE 196

| Exam- | | | MS | | Information |
|---|---|---|---|---|---|
| ple | Chemical structure | NMR | M + H | M − H | of structure |
| F-247 | | (400 MHz, CDCl3) 0.85-0.98 (m, 3H), 1.09-1.32 (m, 7H), 1.57- 1.73 (m, 7H), 1.91-1.99 (m, 1H), 2.03-2.11 (m, 1H), 2.14 (s, 3H), 2,74-2.78 (m, 2H), 2.92-3.03 (m, 2H), 3.17 (dd, J = 14.31, 9.67 Hz, 1H), 3.65-3.78 (m, 2H), 4.02- 3.95 (m, 1H),7.04 (dd, J = 9.27, 8.06 Hz, 1H), 7.17 (d, J = 8.06 Hz, 1H), 7.37 (t, J = 7.25 Hz, 1H), 8.66 (s, 1H) | 459 | 457 | Racemic form |
| F-248 | | (400 MHz, CDCl3) 0.87-0.97 (m, 3H), 1.08-1.30 (m, 8H), 1.58- 1.75 (m, 6H), 1.92-2.00 (m, 1H), 2.07 (s, 3H), 2.07-2.14 (m, 1H), 2.25 (s, 3H), 2.75-2.79 (m, 2H), 2.87 (dd, J = 13.90, 5.04 Hz, 1H), 2.97-3.02 (m, 1H), 3.11 (dd, J = 14.10, 10.07 Hz, 1H), 3.65-3.78 (m, 2H), 3.95-4.02 (m, 1H), 6.94- 6.92 (m, 2H), 7.40 (d, J = 8.46 Hz, 1H), 7.81 (s, 1H) | 439 | 437 | Racemic form |
| F-249 | | (400 MHz, CDCl3) 0.77-0.82 (m, 1H), 0.90-1.02 (m, 3H), 1.07- 1.35 (m, 6H), 1.62-1.78 (m, 7H), 1.84-1.92 (m, 1H), 1.98-2.06 (m, 1H), 2.33 (s, 3H), 2.60 (dd, J = 14.10, 5.64 Hz, 1H), 2.68-2.72 (m, 2H), 2.83 (dd, J = 14.10, 9.27 Hz, 1H), 2.88-2.93 (m, 1H), 3.57-3.67 (m, 2H), 3.82 (s, 3H), 3.87-3.94 (m, 1H), 4.21-4.30 (m, 2H), 6.27 (t, J = 4.84 Hz, 1H), 6.63- 6.67 (m, 2H), 6.98 (d, J = 5.64 Hz, 1H) | 409 | 467 | Racemic form |

TABLE 197

| | | | | | Infor- |
|---|---|---|---|---|---|
| | | | | | mation |
| Exam- | | | MS | | of |
| ple | Chemical structure | NMR | M + H | M − H | structure |
| F-250 | | (400 MHz, CDCl3) 0.91-1.05 (m, 4H), 1.16-1.23 (m, 2H), 1.23- 1.41 (m, 3H), 1.66-1.75 (m, 2H), 1.86-2.00 (m, 5H), 2.29 (s, 3H), 2.80 (t, J = 8.05 Hz, 2H), 2.97 (tt, J = 7.12, 3.96 Hz, 1H), 3.09 (t, J = 6.29 Hz, 2H), 3.17 (t, J = 6.29 Hz, 2H), 7.08 (dd, J = 7.94, 7.72 Hz, 1H), 7.16 (d, J = 7.94 Hz, 1H), 7.62 (d, J = 7.72 Hz, 1H), | 483 | 481 | |
| | | 8.79 (brs, 1H) | | | |
| F-251 | | (400 MHz, CDCl3) 0.89-1.06 (m, 4H), 1.13-1.19 (m, 2H), 1.24- 1.43 (m, 3H), 1.68-1.77 (m, 2H), 1.88-2.03 (m, 5H), 2.33 (s, 3H), 2.74-2.79 (m, 2H), 2.80 (t, J = 7.23 Hz, 2H), 2.93 (tt, J = 7.12, 3.96 Hz, 1H), 3.09 (t, J = 7.23 Hz, 2H), 3.83 (s, 3H), 4.35 (d, J = 5.95 Hz, 2H), 6.39 (brs, 1H), 6.67 (s, 1H), 6.70 (d, J = 7.61 Hz, 1H), 7.08 (d, J = 7.61 Hz, 1H) | 493 | 491 | |
| F-252 | | (400 MHz, DMSO-D6) 0.86 (d, J = 6.62 Hz, 6H), 1.04-1.11 (m, 4H), 1.19-1.26 (m, 2H), 1.51- 1.59 (m, 1H), 1.64-1.72 (m, 2H), 2.02 (s, 3H), 2.22 (s, 3H), 2.59- 2.74 (m, 4H), 2.77-2.88 (m, 2H), 3.06-3.14 (m, 1H), 3.90-4.00 (m, 1H), 6.91 (d, J = 7.72 Hz, 1H), 6.97 (s, 1H), 7.12 (d, J = 8.16 Hz, 1H), 9.25 (s, 1H), 12.18 (brs, 1H) | 427 | 425 | Racemic form |

TABLE 198

| | | | | | Infor- |
|---|---|---|---|---|---|
| | | | | | mation |
| Exam- | | | MS | | of |
| ple | Chemical structure | NMR | M + H | M − H | structure |
| F-253 | | (400 MHz, CDCl3) 0.88 (d, J = 6.62 Hz, 6H), 1.05-1.32 (m, 6H), 1.50-1.80 (m, 3H), 2.09 (s, 3H), 2.25 (s, 3H), 2.70-2.84 (m, 4H), 2.93 (d, J = 7.28 Hz, 2H), 3.03-3.11 (m, 1H), 4.14-4.24 (m, 1H), 5.36 (brs, 1H), 6.48 (brs, 1H), 6.91-6.97 (m, 2H), 7.40 (d, J = 7.94 Hz, 1H), 8.14 (brs, 1H) | 428 | 424 | Racemic form |
| F-254 | | (400 MHz, CDCl3) 0.85-1.01 (m, 2H), 1.24-1.83 (m, 10H), 2.06 (ddd, J = 18.97, 11.91, 8.49 Hz, 3H), 2.27 (s, 3H), 2.80 (t, J = 7.94 Hz, 2H), 2.95-3.00 (m, 1H), 3.10 (t, J = 6.06 Hz, 2H), 3.17 (t, J = 6.06 Hz, 2H), 7.07 (t, J = 8.05 Hz, 1H), 7.17 (d, J = 7.94 Hz, 1H), 7.55 (t, J = 6.95 Hz, 1H), 8.92 (s, 1H) | 451 | 449 | |
| F-255 | | (400 MHz, CDCl3) 0.97-1.92 (m, 13H), 2.30 (s, 3H), 2.78-2.83 (m, 2H), 2.94-2.97 (m, 5H), 3.04- 3.08 (m, 5H), 3.17 (t, J = 5.95 Hz, 2H), 7.08 (t, J = 8.16 Hz, 1H), 7.16 (d, J = 7.72 Hz, 1H), 7.65 (d, J = 8.16 Hz, 1H), 8.72 (s, 1H) | 472 | 470 | |
| F-256 | | (400 MHz, CDCl3) 0.96 (dt, J = 8.23, 3.53 Hz, 2H), 1.17-1.23 (m, 2H), 1.26-1.36 (m, 2H), 1.45 (s, 1H), 1.66-1.79 (m, 6H), 2.08 (ddd, J = 15.27, 8.22, 4.69 Hz, 2H), 2.19 (s, 3H), 2.26 (s, 3H), 2.72-2.83 (m, 2H), 2.94-2.99 (m, 1H), 3.05 (t, J = 6.40 Hz, 2H), 3.16 (q, J = 7.65 Hz, 2H), 6.98 (d, J = 14.11 Hz, 2H), 7.60 (t, | 431 | 429 | |
| | | J = 4.41 Hz, 1H), 8.17 (s, 1H) | | | |

TABLE 199

| | | | | | Infor- |
|---|---|---|---|---|---|
| | | | | | mation |
| Exam- | | | MS | | of |
| ple | Chemical structure | NMR | M + H | M − H | structure |
| F-257 | | (400 MHz, CDCl3) 0.90-0.97 (m, 2H), 1.17 (ddd, J = 22.72, 10.59, 5.84 Hz, 2H), 1.32 (td, J = 15.38, 6.10 Hz, 2H), 1.48 (s, 1H), 1.63- 1.86 (m, 6H), 2.09 (ddd, J = 15.82, 8.66, 3.58 Hz, 2H), 2.33 (s, 3H), 2.79 (q, J = 7.20 Hz, 4H), 2.95 (ddd, J = 16.65, 9.26, 5.73 Hz, 1H), 3.10 (t, J = 7.17 Hz, 2H), 3.81 (s, 3H), 4.37 (t, J = 8.82 Hz, 2H), 6.40 (s, 1H), 6.69 (t, J = 7.61 Hz, 2H), 7.09 (t, J = 7.28 Hz, 1H) | 461 | 459 | |
| F-258 | | (400 MHz, CDCl3) 0.99-1.69 (m, 15H), 2.30 (s, 3H), 2.45-2.49 (m, 1H), 2.78-2.83 (m, 2H), 2.92- 2.99 (m, 4H), 3.04-3.08 (m, 5H), 3.17 (t, J = 5.95 Hz, 2H), 7.08 (t, J = 8.16 Hz, 1H), 7.16 (d, J = 7.72 Hz, 1H), 7.65 (d, J = 8.16 Hz, 1H), 8.72 (s, 1H) | 488 | 484 | |
| F-259 | | (400 MHz, CDCl3) 0.91 (t, J = 3.97 Hz, 2H), 1.11 (t, J = 6.62 Hz, 6H), 1.19 (dd, J = 6.84, 4.41 Hz, 2H), 2.28 (s, 3H), 2.44-2.50 (m, 1H), 2.96-2.99 (m, 1H), 3.13- 3.18 (m, 4H), 4.00 (dd, J = 10.70, 4.52 Hz, 1H), 4.18 (t, J = 7.94 Hz, 1H), 4.39 (t, J = 9.04 Hz, 1H), 4.49 (t, J = 8.49 Hz, 1H), 4.75 (t, J = 7.17 Hz, 1H), 7.08 (t, J = 8.05 | 430 | 428 | |
| | | Hz, 1H), 7.17 (d, J = 7.94 Hz, 1H), | | | |
| | | 7.57 (d, J = 7.94 Hz, 1H), 8.51 | | | |
| | | (s, 1H) | | | |

TABLE 200

| Exam- | | | MS | | Information |
|---|---|---|---|---|---|
| ple | Chemical structure | NMR | M + H | M − H | of structure |
| F-260 | | (400 MHz, CDCl3) 0.89 (d, J = 6.62 Hz, 8H), 1.15-1.18 (m, 2H), 1.58-1.65 (m, 1H), 2.28- 2.30 (m, 5H), 2.90-2.93 (m, 1H), 3.13-3.15 (m, 4H), 3.32 (t, J = 7.50 Hz, 2H), 3.80 (t, J = 7.28 Hz, 2H), 3.90 (dd, J = 15.66, 7.94 Hz, 1H), 7.08 (t, J = 7.94 Hz, 1H), 7.17 (d, J = 7.94 Hz, 1H), 7.56 (d, J = 7.94 Hz, 1H), 8.92 (s, 1H) | 416 | 414 | |
| F-261 | | (400 MHz, CDCl3) 0.97-0.99 (m, 2H), 1.19-1.26 (m, 2H), 1.93- 2.03 (m, 4H), 2.27 (s, 3H), 2.60 (t, J = 5.51 Hz, 4H), 2 87 (t, J = 7.06 Hz, 2H), 2.96-3.00 (m, 3H), 3.11 (d, J = 6.40 Hz, 2H), 3.16 (d, J = 6.40 Hz, 2H), 7.08 (t, J = 7.94 Hz, 1H), 7.17 (d, J = 7.72 Hz, 1H), 7.57 (d, J = 7.94 Hz, 1H), 8.91 (s, 1H) | 452 | 450 | |
| F-262 | | (400 MHz, CDCl3) 0.92-1.00 (m, 8H), 1.12-1.21 (m, 2H),1.35- 1.48 (m, 1H), 1.63-1.71 (m, 2H), 2.16 (s, 3H), 2.24 (s, 3H), 2.74- 2.81(m, 2H), 2.81-2.88 (m, 2H), 2.92-2.99 (m, 1H), 3.07-3.14 (m, 2H), 3.71 (s, 3H), 4.38-4.43 (m, 2H), 6.61 (brs, 1H), 6.84-6.89 (m, 1H), 6.92-6.98 (m, 1H) | 399 | 397 | |
| F-263 | | (400 MHz, CDCl3) 0.87-0.94 (m, 2H), 0.94-0.98 (m, 6H), 1.12- 1.19 (m, 2H), 1.63-1.69 (m, 3H), 2.71-2.79 (m, 2H), 2.84-2.89 (m, 2H), 2 89-2.96 (m, 1H), 3.06- 3.12 (m, 2H), 3.89 (s, 3H), 4.38- 4.44 (m, 2H), 6.85-6.93 (m, 1H), 7.02 (brs, 1H), 7.11-7.16 (m, 1H), 7.27-7.30 (m, 1H) | 439 | 437 | |

TABLE 201

| | | | | | Infor- |
|---|---|---|---|---|---|
| | | | | | mation |
| Exam- | | | MS | | of |
| ple | Chemical structure | NMR | M + H | M − H | structure |
| F-264 | | (400 MHz, DMSO-D6) 0.95-1.04 (m, 4H), 2.21 (t, J = 14.89 Hz, 3H), 2.88 (t, J = 7.28 Hz, 2H), 3.04-3.08 (m, 3H), 3.42 (s, 2H), 7.17 (t, J = 7.94 Hz, 1H), 7.25 (d, J = 7.50 Hz, 1H), 7.34 (d, J = 7.28 Hz, 1H), 9.87 (s, 1H) | 363 | 361 | |
| F-265 | | (400 MHz, CDCl3) 0.88 (d, J = 6.85 Hz, 6H), 1.05-1.09 (m, 2H), 1.20-1.30 (m, 5H), 1.71- 1.79 (m, 2H), 2.11 (s, 3H), 2.25 (s, 3H), 2.70-2.77 (m, 7H), 2.91 (d, J = 7.25 Hz, 2H), 3.03-3.09 (m, 1H), 4.13-4.20 (m, 1H), 6.28 (s, 1H), 6.93-6.95 (m, 2H), 7.44 (d, J = 8.06 Hz, 1H), 8.14 (s, 1H) | 440 | 438 | Racemic form |
| F-266 | | (400 MHz, CDCl3) 0.87 (d, J = 6.45 Hz, 6H), 1.07-1.17 (m, 4H), 1.22-1.28 (m, 2H), 1.53- 1.64 (m, 1H), 1.68-1.76 (m, 2H), 2.08 (s, 3H), 2.25 (s, 3H), 2.71- 2.92 (m, 5H), 2.88 (s, 3H), 2.97 (s, 3H), 3.04-3.16 (m, 2H), 4.25- 4.32 (m, 1H), 6.93-6.94 (m, 2H), 7.46 (d, J = 8.46 Hz, 1H), 7.63 (s, 1H) | 454 | 452 | Racemic form |
| F-267 | | (400 MHz, CDCl3) 0.56-0.66 (m, 1H), 0.79-1.35 (m, 12H), 1.54- 1.88 (m, 9H), 1.88-1.98 (m, 1H), 2.02 (s, 3H), 2.21-2.34 (m, 4H), 2.52-2.68 (m, 2H), 2.74-3.07 (m, 2H), 3.64-3.70 (m, 1H), 3.82 (s, 3H), 4.02-4.19 (m, 2H), 6.17- 6.25 (m, 1H), 6.57-6.64 (m, 2H), 6.82-6.87 (m, 1H) | 494 | 492 | |

TABLE 202

| | | | | | Infor- |
|---|---|---|---|---|---|
| | | | | | mation |
| Exam- | | | MS | | of |
| ple | Chemical structure | NMR | M + H | M − H | structure |
| F-268 | | (400 MHz, CDCl3) 0.97 (dq, J = 9.98, 3.31 Hz, 5H), 1.18 (t, J = 6.40 Hz, 2H), 1.27-1.34 (m, 5H), 1.47-1.68 (m,7H), 2.29 (s, 3H), 2.75 (dt, J = 8.53, 4.25 Hz, 2H), 2.98 (ddd, J = 14.83, 7.33, 3.80 Hz, 1H), 3.08 (t, J = 6.29 Hz, 2H), 3.15- 3.20 (m, 2H), 7.08 (t, J = 7.94 Hz, 1H), 7.16 (d, J = 7.94 Hz, 1H), 7.62 (d, J = 7.94 Hz, 1H), 8.89 (s, 1H) | 415 | 413 | |
| F-269 | | (400 MHz, CDCl3) 0.91-0.99 (m, 5H), 1.19 (dt, J = 13 38, 4.69 Hz, 2H), 1.30 (dd, J = 12.35, 7.06 Hz, 5H), 1.47 (d, J = 5.29 Hz, 5H), 1.69 (t, J = 4.41 Hz, 2H), 2.19 (t, J = 6.29 Hz, 3H), 2.26 (s, 3H), 2.73- 2.77 (m, 2H), 2.95-3.00 (m, 1H), 3.05 (t, J = 6.40 Hz, 2H), 3.17 (t, J = 6.40 Hz, 2H), 6.96 (s, 2H), 7.61 (dd, J = 8.71, 4.74 Hz, 1H). 8.27 (s, | 409 | 407 | |
| | | 1H) | | | |
| F-270 | | (400 MHz, CDCl3) 0.91-0.94 (m, 5H), 1.13-1.19 (m, 2H), 1.32 (t, J = 5.51 Hz, 5H), 1.47 (d, J = 5.29 Hz, 5H), 1.71 (ddd, J = 20.29, 11.91, 6.18 Hz, 2H), 2.33 (d, J = 5.51 Hz, 3H), 2.70-2.75 (m, 2H), 2.80 (t, J = 7.17 Hz, 2H), 2.91-2.97 (m, 1H), 3.11 (q, J = 7.13 Hz, 2H), 3.83 (s, 3H), 4.35 (d, J = 5.73 Hz, 2H), 6.44 (s, 1H), 6.69 (t, J = 8.05 Hz, 2H), 7.09 (t, J = 7.50 Hz, 1H) | 439 | 437 | |

TABLE 203

| | | | | | Infor- |
|---|---|---|---|---|---|
| | | | | | mation |
| Exam- | | | MS | | of |
| ple | Chemical structure | NMR | M + H | M − H | structure |
| F-271 | | (400 MHz, CDCl3) 0.89 (d, J = 6.72 Hz, 6H), 0.90-0.98 (m, 1H), 1.03-1.11 (m, 1H), 1.15- 1.32 (m, 4H), 1.51-1.61 (m, 1H), 1.73-1.82 (m, 2H), 2.12 (s, 3H), 2.26 (s, 3H), 2.76 (dd, J = 8.58, 6.26 Hz, 2H), 2.92-3.09 (m, 3H), 3.37 (brs, 1H), 3.87-3.98 (m, 3H), 6.93-6 98 (m, 2H), 7.46 (d, J = 8.81 Hz, 1H), 7.56 (brs, 1H) | 399 | 397 | Racemic form |
| F-272 | | (400 MHz, CDCl3) 0.84-0.91 (m, 7H), 1.02-1.12 (m, 3H), 1.23- 1.29 (m, 3H), 1.54-1.59 (m, 1H), 1.69-1.77 (m, 2H), 2.11 (s, 3H), 2.17-2.27 (m, 1H), 2.24 (s, 3H), 2.68-2.74 (m, 2H), 2 93 (dd, J = 14.00, 4.08 Hz, 1H), 3.01-3.07 (m, 1H), 3.24 (dd, J = 13.78, 10.48 Hz, 1H), 3.68-3.88 (m, 3H), 6.90-6.95 (m, 2H), 7.48 (d, J = 8.82 Hz, 1H), 7.71 (dd, J = 5.51, 3.09 Hz, 2H), 7.74 (brs, 1H), 7.82 (dd, J = 5.40, 3.20 Hz, 2H) | 542 | 540 | Racemic form |
| F-273 | | (400 MHz CDCl3) 0.85-0.95 (m, 7H), 1.11-1.19 (m, 3H), 1.23- 1.29 (m, 2H), 1.51-1.60 (m, 1H), 1.70- 1.79 (m, 2H), 1.87-2.01 (m, 2H), 2.06 (s, 3H), 2.24 (s, 3H), 2.71-2.78 (m, 4H), 2.82 (dd, J = 13.78, 4.30 Hz, 1H), 2.94-3.01 (m, 1H), 3.10 (dd, J = 13.89, 10.59 Hz, 1H), 3.81-3.89 (m, 1H), 6.89-6.96 (m, 2H), 7.40 (d, J = 8.60 Hz, 1H), 7.82 (brs, 1H) | 412 | 410 | Racemic form |

TABLE 204

| | | | | | Infor- |
|---|---|---|---|---|---|
| | | | | | mation |
| Exam- | | | MS | | of |
| ple | Chemical structure | NMR | M + H | M − H | structure |
| F-274 | | (400 MHz, CDCl3) 0.88 (d, J = 6.62 Hz, 6H), 0.94-1.00 (m, 1H), 1.07-1.13 (m, 1H), 1.15- 1.22 (m, 2H), 1.24-1.30 (m, 2H), 1.52 -1.61 (m, 1H), 1.72-1.81 (m, 2H), 2.08 (s, 3H), 2.25 (s, 3H), 2.75 (t, J = 7.94 Hz, 2H), 2.92 (dd, J = 13.89, 5.07 Hz, 1H), 2.99-3.16 (m, 4H), 3.79-3.87 (m, 1H), 6.90- 6.97 (m, 2H), 7.44 (d, J = 8.60 Hz, 1H), 7.93 (brs, 1H) | 398 | 396 | Racemic form |
| F-275 | | (400 MHz, CDCl3) 0.86-0.91 (m, 7H), 1.07-1.31 (m, 5H), 1.54- 1.61 (m, 1H), 1.72-1.81 (m, 2H), 1.91 (s, 3H), 1.92-2.00 (m, 1H), 2.07-2.16 (m, 1H), 2.09 (s, 3H), 2.25 (s, 3H), 2.71-2.77 (m, 2H), 2.86 (dd, J = 14.11, 4.63 Hz, 1H), 2.98-3.05 (m, 1H), 3.12 (dd, J = 14.11, 9.70 Hz, 1H), 3.20-3.29 (m, 1H), 3.37-3.46 (m, 1H), 3.75- 3.83 (m, 1H), 6.05 (brs, 1H), 6.92- 6.97 (m, 2H), 7.43 (d, J = 8.60 Hz, 1H), 7.98 (brs, 1H) | 454 | 452 | Racemic form |
| F-276 | | (400 MHz, CDCl3) 0.88 (d, J = 6.62 Hz, 6H), 0.93-0.99 (m, 1H), 1.10-1.17 (m, 3H), 1.21- 1.29 (m, 2H), 1.52-1.62 (m, 1H), 1.68-1.78 (m, 2H), 1.86-2.02 (m, 2H), 2.05 (s, 3H), 2.16 (s, 6H), 2.24 (s, 3H), 2.28 (t, J = 7.17 Hz, 2H), 2.71-2.77 (m, 2H), 2.83 (dd, J = 13.78, 4.52 Hz, 1H), 2.94-3.01 (m, 1H), 3.06 (dd, J = 13.76, 10.70 Hz, 1H), 3.78-3.87 (m, 1H), 6.89-6.94 (m, 2H), 7.37 (d, J = 8.38 Hz, 1H), 7.98 (brs, 1H) | 440 | 438 | Racemic form |

TABLE 205

| Exam- | | | MS | | Information |
|---|---|---|---|---|---|
| ple | Chemical structure | NMR | M + H | M − H | of structure |
| F-277 | | (400 MHz, CDCl3) 0.87-0.98 (m, 7H), 1.04-1.13 (m, 1H), 1.20- 1.33 (m, 4H), 1.53-1.64 (m, 1H), 1.75-1.84 (m, 2H), 2.01 (s, 3H), 2.17 (s, 3H), 2.26 (s, 3H), 2.73- 2.80 (m, 2H), 2.85-2.95 (m, 2H) 3.06-3.13 (m, 1H), 3.38 (dt, J = 13.67, 4.85 Hz, 1H), 3.85-3.92 (m, 1H), 3.96-4.04 (m, 1H), 6.87- 7.05 (m, 3H), 7.42 (d, J = 7.94 Hz, 1H), 8.53 (brs, 1H) | 440 | 438 | Racemic form |
| F-278 | | (400 MHz, CDCl3) 0.81-0.96 (m, 7H), 1.08-1.29 (m, 5H), 1.52- 1.62 (m, 1H), 1.71-1.81 (m, 2H), 2.05 (s, 3H), 2.24 (s, 3H), 2.24 (s, 6H), 2.49 (dd, J = 12.35, 6.84 Hz, 1H), 2.62 (dd, J = 12.35, 8.38 Hz, 1H), 2.71-2.78 (m, 2H), 2.96- 3.03 (m, 3H), 3.86-3.93 (m, 1H), 6.89-6.95 (m, 2H), 7.36 (d, J = 8.16 Hz, 1H), 7.76 (brs, 1H) | 426 | 424 | Racemic form |
| F-279 | | (400 MHz, CDCl3) 0.80 (t, J = 5.18 Hz, 1H), 0.91 (d, J = 6.62 Hz, 6H), 1.15 (s, 2H), 1.27-1.32 (m, 2H), 1.57-1.64 (m, 1H), 1.80 (d, J = 3.97 Hz, 2H), 2.04 (s, 3H), 2.30 (s, 3H), 2.65-2.73 (m, 2H), 2.83-2.85 (m, 1H), 3.27 (t, J = 10.92 Hz, 1H), 3.66 (dd, J = 18.53, 10.37 Hz, 1H), 3.76-4.06 (m, 7H), 4.29 (q, J = 6.54 Hz, 3H), 6.63 (t, J = 8.49 Hz, 2H), 6.97 (t, J = 5.73 Hz, 1H), 7.23 (s, 1H) | 482 | 480 | Racemic form |

TABLE 206

| Exam- | | | MS | | Information |
|---|---|---|---|---|---|
| ple | Chemical structure | NMR | M + H | M − H | of structure |
| F-280 | | (400 MHz, CDCl3) 0.90 (d, J = 6.84 Hz, 6H), 0.94-0.98 (m, 2H), 1.04-1.23 (m, 4H), 1.41- 1.51 (m, 2H), 1.62-1.74 (m, 2H), 1.87 (d, J = 10.14 Hz, 2H), 2.00 (d, J = 10.81 Hz, 2H), 2.27 (s, 3H), 2.78 (tt, J = 12.13, 3.46 Hz, 1H), 2.93-2.98 (m, 1H), 3.07 (t, J = 5.73 Hz, 2H), 3.17 (t, J = | 429 | 427 | |
| | | 5.95 Hz, 2H), 7.08 (t, J = 8.05 | | | |
| | | Hz, 1H), 7.16 (d, J = 7.50 Hz, | | | |
| | | 1H), 7.62 (d, J = 8.38 Hz, 1H), | | | |
| | | 8.84 (brs, 1H) | | | |
| F-281 | | (400 MHz, CDCl3) 0.82-0.90 (m, 7H), 1.14-1.30 (m, 5H), 1.53-1.61 (m, 1H), 1.65-1.95 (m, 6H), 2.03 (s, 3H), 2.23 (s, 3H), 2.71-2.80 (m, 3H), 2.98-3.12 (m, 2H), 3.58-3.81 (m, 3H), 6 88-6.93 (m, 2H), 7.37 (d, J = 8.16 Hz, 1H), 7.46 (brs, 1H), 7.71 (dd, J = 5.51, 3.09 Hz, 2H), 7.62 (dd, J = 5.51, 3.09 Hz, 2H) | 556 | 554 | Racemic form |
| F-282 | | (400 MHz, CDCl3) 0.84-0.91 (m, 7H), 1.12-1.30 (m, 5H), 1.37-1.47 (m, 1H), 1.50-1.90 (m, 6H), 2.05 (s, 3H), 2.24 (s, 3H), 2.66-2.77 (m, 4H), 2.81 (dd, J = 13.89, 4.19 Hz, 1H), 2.93-3.00 (m, 1H), 3.10 (dd, J = 13.78, 10.92 Hz, 1H), 3.70-3.78 (m, 1H), 6.90-6.95 (m, 2H), 7.39 (d, J = 8.82 Hz, 1H), 7.77 (brs, 1H) | 426 | 424 | Racemic form |

TABLE 207

| Exam- | | | MS | | Information |
|---|---|---|---|---|---|
| ple | Chemical structure | NMR | M + H | M − H | of structure |
| F-283 | | (400 MHz, CDCl3) 0.82-0.98 (m, 1H), 0.87 (d, J = 6.62 Hz, 6H), 1.15- 1.28 (m, 5H), 1.50-1.60 (m, 1H), 1.68-1.79 (m, 2H), 2.01 (s, 3H), 2.12-2.51 (m, 2H), 2.23 (s, 3H), 2.44-2.52 (m, 2H), 2.69-2.75 (m, 2H), 2.77-2.83 (m, 1H), 2.99- 3.06 (m, 1H), 3.13-3.22 (m, 1H), 3.96-4.05 (m, 1H), 6.88-6.93 (m, 2H), 7.24-7.28 (m, 1H), 8.27 (brs, 1H) | 441 | 439 | Racemic form |
| F-284 | | (400 MHz, CDCl3) 0.85-0.94 (m, 7H), 1.08-1.30 (m, 5H) 1.51- 1.61 (m, 1H), 1.66-1.79 (m, 2H), 2.06 (s, 3H), 2.08-2.20 (m, 2H), 2.24 (s, 3H), 2.26-2.39 (m, 2H), 2.72 (t, J = 7.83 Hz, 2H), 2.84 (dd, J = 14.22, 4.74 Hz, 1H), 3.00-3.07 (m, 1H), 3.13 (dd, J = 14.22, 10.03 Hz, 1H), 3.83-3.91 (m, 1H), 5.59 (brs, 1H), 6.33 (brs, 1H), 6.89- 6.96 (m, 2H), 7.33 (d, J = 7.94 Hz, 1H), 8.51 (brs, 1H) | 440 | 438 | Racemic form |
| F-295 | | (400 MHz, CDCl3) 0.85-0.93 (m, 7H), 1.06-1.30 (m, 5H), 1.51- 1.62 (m, 1H), 1.69-1.81 (m, 2H), 2.00-2.34 (m, 10H), 2.68-2.76 (m, 5H), 2.85 (dd, J = 14.11, 4.85 Hz, 1H), 3.01-3.16 (m, 2H), 3.77-3.87 (m, 1H), 6.21 (brs, 1H), 6.89-6.97 (m, 2H), 7.36 (d, J = 7.94 Hz, 1H), 8.49 (brs, 1H) | 454 | 452 | Racemic form |

TABLE 208

| | | | | | Infor- |
|---|---|---|---|---|---|
| | | | | | mation |
| Exam- | | | MS | | of |
| ple | Chemical structure | NMR | M + H | M − H | structure |
| F-286 | | (400 MHz, CDCl3) 0.87-0.94 (m, 7H), 1.04-1.32 (m, 5H), 1.53- 1.63 (m, 1H), 1.71-1.81 (m, 2H), 2.09 (s, 3H), 2.11-2.22 (m, 2H), 2.24 (s, 3H), 2.33-2.44 (m, 2H), 2.71-2.83 (m, 3H), 2.90 (s, 3H), 2.98 (s, 3H), 3.02-3.08 (m, 1H), 3.14 (dd, J = 13.67, 10.81 Hz, 1H), 3.80-3.88 (m, 1H), 6.91-6.96 (m, 2H), 7.46 (d, J = 8.60 Hz, 1H), 8.12 (brs, 1H) | 468 | 466 | Racemic form |
| F-287 | | (400 MHz, CDCl3) 0.83-0.91 (m, 7H), 1.12-1.30 (m, 5H), 1.39- 1.50 (m, 1H), 1.52-1.67 (m, 2H), 1.70-1.91 (m, 2H), 1.93 (s, 3H), 2.05 (s, 3H), 2.24 (s, 3H), 2.70- 2.77 (m, 2H), 2.84 (dd, J = 14.00, 4.52 Hz, 1H), 2.96-3.08 (m, 2H), 3.16-3.23 (m, 2H), 3.70-3.78 (m, 1H), 5.88 (brs, 1H), 6.89-6.96 (m, 2H), 7.37 (d, J = 8.16 Hz, 1H), 8.09 (brs, 1H) | 468 | 466 | Racemic form |
| F-288 | | (400 MHz, CDCl3) 0.85-0.92 (m, 7H), 1.12-1.31 (m, 5H), 1.34- 1.46 (m, 1H), 1.51-1.63 (m, 2H), 1.69-1.83 (m, 4H), 2.04 (s, 3H), 2.10-2.26 (m, 2H), 2.19 (s, 3H), 2.24 (s, 3H), 2.74 (t, J = 7.88 Hz, 2H), 2.82 (dd, J = 13.80, 4.29 Hz, 1H), 2.94-3.02 (m, 1H), 3.12 (dd, J = 13.80, 11.02 Hz, 1H), 3.71-3.78 (m, 1H), 6.89-6.94 (m, 2H), 7.36 (d, J = 8.12 Hz, 1H), 8.01 (brs, 1H) | 454 | 452 | Racemic form |

TABLE 209

| | | | | | Infor- |
|---|---|---|---|---|---|
| | | | | | mation |
| Exam- | | | MS | | of |
| ple | Chemical structure | NMR | M + H | M − H | structure |
| F-289 | | (400 MHz, CDCl3) 0.86 (d, J = 6.62 Hz, 6H), 0.89-0.96 (m, 2H), 1.13-1.23 (m, 2H), 1.35 (t, J = 7.06 Hz, 2H), 1.50-1.60 (m, 1H), 2.03-2.12 (m, 1H), 2.13 (s, 3H), 2.14 (s, 3H), 2.25 (s, 3H), 2.35-2.63 (m, 4H), 2.85-2.95 (m, 1H), 3.25-3.35 (m, 1H), 3.39- 3.50 (m, 1H), 3.56-3.75 (m, 1H), 3.84-3.98 (m, 1H), 4.16-4.28 (m, 2H), 4.39-4.48 (m, 1H), 6.94 (s, 1H), 6.95 (d, J = 7.94 Hz, 1H), 7.72 (d, J = 7.94 Hz, 1H) 8.96 (brs, 1H) | 478 | 476 | Racemic form |
| F-290 | | (400 MHz, CDCl3) 0.94-1.03 (m, 2H), 1.16 (dd, J = 13.80, 8.45 Hz, 6H), 1.23-1.28 (m, 2H), 1.55 (tt, J = 13.68, 4.96 Hz, 2H), 1.81-1.89 (m, 2H), 2.14 (s, 3H), 2.15 (s, 3H), 2.26 (t, J = 10.55 Hz, 3H), 2.82 (ddd, J = 17.22, 9.22, 6.20 Hz, 2H), 2.97- 3.03 (m, 1H), 3.16 (s, 3H), 3.26- 3.34 (m, 1H), 3.75 (dt, J = 18.01, 6.09 Hz, 1H), 3.89-3.97 (m, 1H), 4.18-4.22 (m, 2H), 4.44 (dd, J = 11.59, 8.35 Hz, 1H), 6.96 (dd, J = 5.80, 5.33 Hz, 2H), 7.72 (d, J = 8.12 Hz, 1H), 9.04 (s, 1H) | 482 | 480 | Racemic form |

TABLE 210

| | | | | | Infor- |
|---|---|---|---|---|---|
| | | | | | mation |
| Exam- | | | MS | | of |
| ple | Chemical structure | NMR | M + H | M − H | structure |
| F-291 | | (400 MHz, MeOH-D4) 0.90 (d, J = 6.62 Hz, 6H), 1.07-1.22 (m, 4H), 1.24-1.33 (m, 2H), 1.54- 1.65 (m, 1H), 1.74-1.83 (m, 2H), 1.96 (s, 3H), 2.07 (s, 3H), 2.25 (s, 3H), 2.84 (t, J = 7.83 Hz, 2H), 3.03 (dd, J = 15.11, 5.84 Hz, 1H), 3.10- 3.16 (m, 1H), 3.26 (dd, J = 15.00, 9.48 Hz, 1H), 5.88 (dd, J = 9.48, 5.95 Hz, 1H), 6.93 (d, J = 7.94 Hz, 1H), 6.99 (s, 1H), 7.06 (d, J = 7.94 Hz, 1H) | 426 | 424 | |
| F-292 | | (400 MHz, CDCl3) 0.94-1.02 (m, 2H), 1.19-1.28 (m, 2H), 2.21- 2.34 (m, 2H), 2.30 (s, 3H), 2.80 (t, J = 6.92 Hz, 2H), 2.90-3.04 (m, 7H), 3.06-3.22 (m, 4H), 7.09 (dd, J = 8.12, 7.88 Hz, 1H), 7.17 (d, J = 8.12 Hz, 1H), 7.63 (d, J = 7.88 Hz, 1H), 8.76 (s, 1H) | 454 | 452 | |
| F-293 | | (400 MHz, CDCl3) 0.88 (d, J = 6.40 Hz, 6H), 0.89-0.97 (m, 2H), 1.11-1.21 (m, 2H), 1.44 (t, J = 6.84 Hz, 2H), 1.51-1.61 (m, 1H), 2.04-2.12 (m, 1H), 2.13 (s, 3H), 2.14 (s, 3H), 2.25 (s, 3H), 2.47-2.66 (m, 4H), 2.86-2.94 (m, 1H), 3.26-3.35 (m, 1H), 3.56- 3.75 (m, 2H), 3.85-3.98 (m, 1H), 4.17-4.28 (m, 2H), 4.40-4.48 (m, 1H), 6.94 (s, 1H), 6.96 (d, J = 8.16 Hz, 1H), 7.73 (d, J = 8.16 Hz, 1H), 8.98 (brs, 1H) | 478 | 476 | Racemic form |

TABLE 211

| | | | | | Infor- |
|---|---|---|---|---|---|
| | | | | | mation |
| Exam- | | | MS | | of |
| ple | Chemical structure | NMR | M + H | M − H | structure |
| F-294 | | (400 MHz, CDCl3) 0.99-1.01 (m, 2H), 1.16-1.26 (m, 10H), 1.72 (brs, 3H), 2.31 (s, 3H), 2.45 (brs, 2H), 3.07-3.21 (m, 8H), 7.09 (t, J = 8.12 Hz, 1H), 7.17 (d, J = 7.88 Hz, 1H), 7.63 (d, J = 7.65 Hz, 1H), 8.72 (s, 1H) | 418 | 416 | |
| F-295 | | (400 MHz, CDCl3) 0.80-0.96 (m, 3H), 1.08-1.33 (m, 3H), 1.39- 1.58 (m, 2H), 1.58-1.74 (m, 5H), 1.74-1.95 (m, 7H), 2.02 (s, 3H), 2.10-2.19 (m, 4H), 2.23 (s, 3H), 2.38-2.49 (m, 1H), 2.71-2.80 (m, 2H), 2.88-2.97 (m, 1H), 3.04- 3.19 (m, 2H), 3.91-3.99 (m, 1H), 6.87-6.93 (m, 2H), 7.34-7.41 (m, 1H), 7.54-7.69 (m, 1H) | 464 | 462 | |
| F-296 | | (400 MHz, CDCl3) 0.87-0.93 (m, 11H), 1.08-1.19 (m, 2H), 1.29 (dd, J = 10.09, 7.30 Hz, 2H), 1.77 (td, J = 15.07, 7.50 Hz, 2H), 2.04 (s, 3H), 2.04 (s, 3H), 2.28 (s, 3H), 2.41- 2.48 (m, 1H), 2.55-2.62 (m, 1H), 2.75 (t, J = 7.88 Hz, 2H), 3.33-3.38 (m, 1H), 3.73-3.95 (m, 3H), 5.56 (d, J = 7.88 Hz, 1H), 6.97-7.01 (m, 2H), 7.24 (brs, 1H), 7.51 (d, J = 8.12 Hz, 1H) | 466 | 464 | |

TABLE 212

| Exam- | | | MS | | Information |
|---|---|---|---|---|---|
| ple | Chemical structure | NMR | M + H | M − H | of structure |
| F-297 | | (400 MHz, CDCl3) 0.65-0.75 (m, 1H), 0.85-1.11 (m, 2H), 0.90 (s, 9H), 1.26-1.32 (m, 3H), 1.75- 1.81 (m, 1H), 2.00 (s, 3H), 2.28- 2.41 (m, 2H), 2.34 (s, 3H), 2.68 (t, J = 7.88 Hz, 2H), 3.15-3.20 (m, 1H), 3.39-3.46 (m, 1H), 3.76- 3.85 (m, 2H), 3.78 (s, 3H), 4.26 (dd, J = 14.15, 5.33 Hz, 1H), 4.38 (dd, J = 14.26, 6.61 Hz, 1H), 5.38 (d, J = 7.65 Hz, 1H), 6.16 (t, J = 6.03 Hz, 1H), 6.64 (s, 1H), 6.69 (d, J = 7.42 Hz, 1H), 7.04 (d, J = 7.65 Hz,1H) | 4796 | 494 | |
| F-298 | | (400 MHz, CDCl3) 0.78-1.00 (m, 8H), 1.12-1.24 (m, 2H), 1.47- 1.67 (m, 1H), 1.92-2.23 (m, 9H), 2.25 (s, 3H), 2.40-2.54 (m, 2H), 2.85-2.96 (m, 1H), 3.25-3.44 (m, 1.5H), 3.46-3.57 (m, 0.5H), 3.63- 3.75 (m, 0.8H), 3.84-3.97 (m, 1.2H), 3.99-4.11 (m, 0.4H), 4.13- 4.29 (m, 1.8H), 4.37-4.50 (m, 0.8H), 6.91-7.00 (m, 2H), 7.59 (d, J = 7.88 Hz, 0.2H), 7.73 (d, J = 8.12 Hz, 0.8H), 8.07 (s, 0.2H), 8.98 (s, 0.8H) | 464 | 462 | Mixtures of four stereoisomers (Stereo- chemistry on Pyrrolidine ring: Mixture of two Trans-isomers) (Stereo- chemistry on Cyclobutane |
| | | | | | ring: |
| | | | | | Mixture of |
| | | | | | Trans-isomer |
| | | | | | and |
| | | | | | Cis-isomer) |

TABLE 213

| | | | | | Infor- |
|---|---|---|---|---|---|
| | | | | | mation |
| Exam- | | | MS | | of |
| ple | Chemical structure | NMR | M + H | M − H | structure |
| F-299 | | (400 MHz, CDCl3) 0.82-1.35 (m, 14H), 1.63 (ddt, J = 18.97, 10.51, 4.16 Hz, 2H), 2.13 (s, 3H), 2.17 (t, J = 5.40 Hz, 3H), 2.21-2.23 (m, 3H), 2.74 (dtt, J = 27.67, 10.86, 3.66 Hz, 3H), 3.01 (ddd, J = 15.11, 7.61, 4.08 Hz, 1H), 3.32 (q, J = 11.03 Hz, 1H), 3.73 (tt, J = 14.67, 5.00 Hz, 1H), 3.94 (tt, J = 14.78, 4.23 Hz, 1H), 4.15-4.27 (m, 2H), 4.36-4.45 (m, 1H), 6.90-7.01 (m, 2H), 7.72 (d, J = 7.94 Hz, 1H), 9.13 (t, J = 29.77 Hz, 1H) | 466 | 464 | Racemic form |
| F-300 | | (400 MHz, CDCl3) 0.88 (d, J = 7.06 Hz, 3H), 0.91 (d, J = 5.95 Hz, 3H), 0.97-1.88 (m, 14H), 2.10-2.12 (m, 6H), 2.24 (d, J = 11.03 Hz, 3H), 2.77 (tt, J = 12.13, 3.42 Hz, 1H), 2.95-3.01 (m, 1H), 331 (t, J = 10.92 Hz, 1H), 3.74 (dt, J = 14.78, 6.23 Hz, 1H), 3.93 (ddd, J = 13.45, 8.71, 4.63 Hz, 1H), 4.16-4.26 (m, 2H), 4.42 (dd, J = 11.47, 8.38 Hz, 1H), 6.95 (d, J = 10.14 Hz, 2H), 7.68 (t, J = 7.83 Hz, 1H), 9.11 (s, 1H) | 492 | 490 | Racemic form |
| F-301 | | (400 MHz, CDCl3) 0.87-0.88 (m, 6H), 0.96-1.04 (m, 1H), 1.12- 1.19 (m, 3H), 1.19-1.31 (m, 2H), 1.21 (s, 3H), 1.27(s, 3H), 1.53- 1.60 (m, 1H), 1.68-1.78 (m, 2H), 1.86 (brs, 1H), 1.92 (dd, J = 14.56, 5.73 Hz, 1H), 2.09 (s, 3H), 2.16 (dd, J = 14.34, 7.50 Hz, 1H), 2.25 (s, 3H), 2.71-2.77 (m, 2H), 2.92- 3.05 (m, 3H), 3.90-3.99 (m, 1H), 6.91-6.97 (m, 2H), 7.43 (d, J = 8.82 Hz, 1H), 7.88 (brs, 1H) | 441 | 439 | Racemic form |

TABLE 214

| | | | | | Infor- |
|---|---|---|---|---|---|
| | | | | | mation |
| Exam- | | | MS | | of |
| ple | Chemical structure | NMR | M + H | M − H | structure |
| F-302 | | (400 MHz, CDCl3) 0.89-0.90 (m, 9H), 1.18-1.34 (m, 5H), 1.79- 1.82 (m, 3H), 2.27 (s, 3H), 2.29 (s, 3H), 2.79 (t, J = 7.94 Hz, 2H), 2.98 (s, 3H), 3.26 (s, 1H), 3.71 (dd, J = 8.82, 3.31 Hz, 2H), 4.20 (d, J = 9.48 Hz, 1H), 4.45 (t, J = 9.04 Hz, 1H), 6.96 (d, J = 12.13 Hz, 2H), 7.66 (d, J = 7.94 Hz, 1H), 9.44 (s, 1H) | 452 | 450 | |
| F-303 | | (400 MHz, CDCl3) 0.87-0.88 (m, 9H), 1.12-1.30 (m, 5H), 1.76 (t, J = 7.06 Hz, 2H), 2.06 (s, 3H), 2.26- 2.28 (m, 4H), 2.76 (t, J = 7.83 Hz, 2H), 2.91 (s, 3H), 3.42-3.46 (m, 1H), 3.65 (t, J = 9.26 Hz, 1H), 3.94 (t, J = 9.37 Hz, 1H), 4.38 (d, J = 11.03 Hz, 1H), 4.58-4.63 (m, 1H), 6.95 (t, J = 5.84 Hz, 2H), 7.60 (d, J = 7.94 Hz, 1H), 8.69 (s, 1H) | 452 | 450 | |
| F-304 | | (400 MHz, CDCl3) 0.97-1.03 (m, 2H), 1.17-1.22 (m, 2H), 1.29 (s, 6H), 2.30 (s, 3H), 2.97-3.03 (m, 1H), 3.05-3.12 (m, 3H), 3.18 (t, J = 6.29 Hz, 2H), 3.49 (q, J = 2.87 Hz, 1H), 3.94 (t, J = 6.62 Hz, 2H), 7.08 (t, J = 8.05 Hz, 1H), 7.16 (d, J = 7.72 Hz, 1H), 7.67 (d, J = 7.50 Hz, 1H), 8.62 (brs, 1H) | 459 | 457 | |
| F-305 | | (400 MHz, CDCl3) 0.90 (s, 9H), 0.95 (d, J = 2.65 Hz, 2H), 1.18 (d, J = 5.73 Hz, 2H), 1.28 (dd, J = 11.14, 5.06 Hz, 2H), 1.73-1.76 (m, 2H), 2.18 (s, 3H), 2.26 (s, 3H), 2.75 (t, J = 7.83 Hz, 2H), 2.96 (s, 1H), 3.06 (t, J = 6.40 Hz, 2H), 3.17 (t, J = 6.40 Hz, 2H), 6.95 (s, 2H), 7.59 (d, J = 8.60 Hz, 1H), 8.38 (s, 1H) | 383 | 381 | |

TABLE 215

| | | | | | Infor- |
|---|---|---|---|---|---|
| | | | | | mation |
| Exam- | | | MS | | of |
| ple | Chemical structure | NMR | M + H | M − H | structure |
| F-306 | | (400 MHz, CDCl3) 0.86-0.90 (m, 2H), 0.87 (s, 9H), 1.12-1.18 (m, 3H), 1.22-1.27 (m, 2H), 1.68- 1.76 (m, 1H), 2.06 (m, 3H) 2.24 (s, 3H), 2.44-2.56 (m, 2H), 2.74 (dd, J = 6.85, 6.85 Hz, 2H), 2.82 (dd, J = 13.70, 4.03 Hz, 1H), 2.93-2.99 (m, 1H), 3.12 (dd, J = 14.10, 11.28 Hz, 1H), 3.77-3.85 (m, 1H), 5.05-5.09 (m, 2H), 5.71-5.81 (m, 1H), 6.91-6.92 (m, 2H), 7.41 (d, J = 8.87 Hz, 1H), 7.64 (s, 1H) | 423 | 421 | Racemic form |
| F-307 | | (400 MHz, CDCl3) 0.87 (s, 9H), 0.93-0.98 (m, 1H), 1.07-1.12 (m, 1H), 1.15-1.20 (m, 2H), 1.24- 1.29 (m, 2H), 1.68-1.76 (m, 2H), 1.94-2.02 (m, 1H), 2.06-2.14 (m, 1H), 2.07 (s, 3H), 2.24 (s, 3H), 2.73 (dd, J = 8.06, 8.06 Hz, 2H), 2.88 (dd, J = 13.90, 5.04 Hz, 1H), 2.98-3.04 (m, 1H), 3.12 (dd, J = 14.31, 9.87 Hz, 1H), 3.66-3.78 (m, 2H), 3.95-4.02 (m, 1H), 6.92- 6.94 (m, 2H) 7.40 (d, J = 8.46 Hz 1H), 7.81 (s, 1H) | 427 | 425 | Racemic form |
| F-308 | | (400 MHz, CDCl3) 0.86-0.92 (m, 9H), 0.93-1.12 (m, 1H), 1.12- 1.22 (m, 1H), 1.24-1.36 (m, 3H), 1.74 -1.99 (m, 3H), 1.99-2.08 (m, 1H), 2.11 (s, 3H), 2.27 (s, 3H), 2.44 -2.58 (m, 1H), 2.71-2.79 (m, 2H), 3.02-3.12 (m, 1H), 3.25- 3.32 (m, 1H), 3.39-3.51 (m, 2H), 3.67-3.82 (m, 1H), 4.08-4.25 (m, 2H), 6.29-6.40 (m, 1H), 6.95- 7.02 (m, 2H), 7.26-7.32 (m, 1H), 7.49 -7.57 (m, 1H) | 496 | 494 | |

TABLE 216

| | | | | | Infor- |
|---|---|---|---|---|---|
| | | | | | mation |
| Exam- | | | MS | | of |
| ple | Chemical structure | NMR | M + H | M − H | structure |
| F-309 | | (400 MHz, CDCl3) 0.84-0.91 (m, 10H), 0.92-1.00 (m, 1H), 1.07- 1.14 (m, 2H), 1.22-1.30 (m, 5H) 1.67-1.86 (m, 2H), 2.00-2.15 (m, 4H), 2.19-2.29 (m, 4H), 2.67- 2.83 (m, 3H), 2.95-3.04 (m, 1H), 3.08-3.21 (m, 2H), 4.27-4.33 (m, 1H), 6.88-6.93 (m, 2H), 7.34- 7.38 (m, 1H), 7.45 (brs, 1H) | 438 | 436 | |
| F-310 | | (400 MHz, CDCl3) 0.81-0.96 (m, 11H), 1.09-1.33 (m, 6H), 1.75- 1.96 (m, 2H), 1.99-2.08 (m, 4H), 2.10-2.19 (m, 4H), 2.23 (s, 3H), 2.38-2.49 (m, 1H), 2.63-2.79 (m, 2H), 2.89-2.97 (m, 1H), 3.07- 3.19 (m, 2H), 3.94-3.99 (m, 1H), 6.87-6.95 (m, 2H), 7.33-7.41 (m, 1H), 7.69 (brs, 1H) | 452 | 450 | |
| F-311 | | (400 MHz, CDCl3) 0.79-0.94 (m, 11H), 1.13-1.34 (m, 5H), 1.65- 1.81 (m, 2H), 1.97 (s, 3H), 2.14- 2.30 (m, 5H), 2.51-2.84 (m, 6H), 2.96 -3.07 (m, 1H), 3.57-3.68 (m, 1H), 5.29-5.36 (m, 1H), 6.88- 6.96 (m, 2H), 7.10-7.23 (m, 1H), 7.93 (brs, 1H) | 466 | 464 | Mixture of two stereoisomers (Mixture of diastereomers) |
| F-312 | | (400 MHz, MeOH-D4) 0.91 (d, J = 6.62 Hz, 6H), 1.04-1.19 (m, 4H), 1.25-1.32 (m, 2H), 1.55- 1.65 (m, 1H), 1.73-1.83 (m, 2H), 1.91 (s, 3H), 2.30 (s, 3H), 2.77- 2.87 (m, 3H), 3.00-3.08 (m, 2H), 3.79 (s, 3H), 4.18 (d, J = 15.00 Hz, 2H), 4.26 (d, J = 15.00 Hz, 2H), 5.77 (dd, J = 9.48, 5.95 Hz, 1H), 6.66 (d, J = 7.50 Hz, 1H), 6.73 (s, 1H), 6.92 (d, J = 7.50 Hz, 1H) | 456 | 454 | |

TABLE 217

| | | | | | Infor- |
|---|---|---|---|---|---|
| | | | | | mation |
| Exam- | | | MS | | of |
| ple | Chemical structure | NMR | M + H | M − H | structure |
| F-313 | | (400 MHz, CDCl3) 0.88 (d, J = 6.62 Hz, 6H), 0.93-0.99 (m, 1H), 1.06-1.31 (m, 5H), 1.52- 1.60 (m, 1H), 1.69-1.81 (m, 2H), 1.94-2.02 (m, 1H), 2.06-2.15 (m, 1H), 2.08 (s, 3H), 2.25 (s, 3H), 2.54 (brs, 1H), 2.74 (t, J = 7.83 Hz, 2H), 2.87 (dd, J = 14.00, 4.74 Hz, 1H), 2.97-3.04 (m, 1H), 3.11 (dd, J = 14.11, 10.14 Hz, 1H), 3.64-3.81 (m, 2H), 3.94-4.03 (m, 1H), 6.91- 6.97 (m, 2H), 7.41 (d, J = 8.60 Hz, 1H), 7.72 (brs, 1H) | 413 | 411 | |
| F-314 | | (400 MHz, CDCl3) 0.88 (d, J = 6.62 Hz, 6H), 0.93-0.99 (m, 1H), 1.06-1.31 (m, 5H), 1.52- 1.60 (m, 1H), 1.69-1.81 (m, 2H), 1.94-2.02 (m, 1H), 2.06-2.15 (m, 1H), 2.08 (s, 3H), 2.25 (s, 3H), 2.54 (brs, 1H), 2.74 (t, J = 7.83 Hz, 2H), 2.87 (dd, J = 14.00, 4.74 Hz, 1H), 2.97-3.04 (m, 1H), 3.11 (dd, J = 14.11, 10.14 Hz, 1H), 3.64-3.81 (m, 2H), 3.94-4.03 (m, 1H), 6.91- 6.97 (m, 2H), 7.41 (d, J = 8.60 Hz, 1H), 7.72 (brs, 1H) | 413 | 411 | |
| F-315 | | (400 MHz, CDCl3) 0.85-1.10 (m, 8H), 1.17-1.35 (m, 4H), 1.53- 1.64 (m, 1H), 1.74-1.84 (m, 2H), 2.02-2.27 (m, 9H), 2.71-4.49 (m, 9H), 6.91-7.00 (m, 2H), 7.56 and 7.74 (each d, J = 8.82 and 8.16 Hz, total 1H), 8.17 and 9.03 (each brs, total 1H) | 452 | 450 | |

TABLE 218

| | | | | | Infor- |
|---|---|---|---|---|---|
| | | | | | mation |
| Exam- | | | MS | | of |
| ple | Chemical structure | NMR | M + H | M − H | structure |
| F-316 | | (400 MHz, CDCl3) 0.85-1.10 (m, 8H), 1.17-1.35 (m, 4H), 1.53- 1.64 (m, 1H), 1.74-1.84 (m, 2H), 2.02-2.27 (m, 9H), 2.71-4.49 (m, 9H), 6.91-7.00 (m, 2H), 7.56 and 7.74 (each d, J = 8.82 and 8.16 Hz, toal 1H), 8.17 and 9.03 (each brs, total 1H) | 452 | 450 | |
| F-317 | | (400 MHz, MeOH-D4) 0.89 (d, J = 6.62 Hz, 6H), 0.92-1.39 (m, 6H), 1.45-1.63 (m, 1H), 1.91- 2.07 (m, 2H), 2.03 (s, 3H) 2.09 and 2.12 (each s, total 3H), 2.26 (s, 3H), 2.41-2.61 (m, 3H), 3.02- 3.11 (m, 1H), 3.41-3.91 (m, 4H), 4.09-4.31 (m, 3H), 6.96 (d, J = 7.94 Hz, 1H), 7.01 (s, 1H), 7.10 and 7.13 (each d, J = 7.94 and 7.94 Hz, total 1H) | 478 | 476 | |
| F-318 | | (400 MHz, MeOH-D4) 0.89 (d, J = 6.62 Hz, 6H), 0.92-1.39 (m, 6H), 1.45-1.63 (m, 1H), 1.91- 2.07 (m, 2H), 2.03 (s, 3H), 2.09 and 2.12 (each s, total 3H), 2.26 (s, 3H), 2.41-2.61 (m, 3H), 3.02- 3.11 (m, 1H), 3.41-3.91 (m, 4H), 4.09-4.31 (m, 3H), 6.96 (d, J = 7.94 Hz, 1H), 7.01 (s, 1H), 7.10 and 7.13 (each d, J = 7.94 and 7.94 Hz, total 1H) | 478 | 476 | |

TABLE 219

| | | | | | Infor- |
|---|---|---|---|---|---|
| | | | | | mation |
| Exam- | | | MS | | of |
| ple | Chemical structure | NMR | M + H | M − H | structure |
| F-319 | | (400 MHz, MeOH-D4) 0.57-0.81 (m, 2H), 1.03-1.18 (m, 2H), 1.28- 1.36 (m, 6H), 2.08 (s, 3H), 2.15 and 2.16 (each s, total 3H), 2.28 (s, 3H), 2.95-3.06 (m, 1H), 3.43- 4.47 (m, 7H), 6.96-7.01 (m, 1H), 7.04 (s, 1H), 7.13-7.22 (m, 1H), 7.40 -7.47 (m, 2H), 7.66-7.74 (m, 2H) | 486 | 484 | Racemic form |
| F-320 | | (400 MHz, MeOH-D4) 0.63-0.75 (m, 2H), 1.03-1.12 (m, 2H), 1.31 (d, J = 6.96 Hz, 6H), 2.17 (s, 3H), 2.27 (s, 3H), 2.94-3.10 (m, 3H), 3.27-3.35 (m, 2H), 3.44-3.52 (m, 1H), 6.97 (d, J = 7.88 Hz, 1H), 7.03 (s, 1H), 7.18 (d, J = 8.12 Hz, 1H), 7.42 (d, J = 8.12 Hz, 2H), 7.69 (d, J = 8.12 Hz, 2H) | 403 | 401 | |
| F-321 | | (400 MHz, CDCl3) 0.84-1.02 (m, 15H), 1.20-1.28 (m, 3H),1.52- 1.59 (m, 1H), 2.14 (s, 3H), 2.16 (s, 3H), 2.25 (s, 3H), 2.68-2.85 (m, 2H), 2.97-3.03 (m, 1H), 3.30 (t, J = 11.03 Hz, 1H), 3.68-3.75 (m, 1H), 3.85-3.94 (m, 1H), 4.15- 4.25 (m, 2H), 4.45 (dd, J = 11.47, 8.38 Hz, 1H), 6.97 (t, J = 10.14 Hz, 2H), 7.75 (d, J = 8.16 Hz, 1H), 9.04 (s, 1H) | 480 | 478 | Racemic form |
| F-322 | | (400 MHz, CDCl3) 0.78-0.92 (m, 11H), 1.17-1.31 (m, 4H), 1.66- 1.80 (m, 2H), 1.97 (s, 3H), 2.22- 2.33 (m, 5H), 2.57-2.82 (m, 5H), 2.95-3.02 (m, 1H), 3.46-3.59 (m, 3H) 5.27-5.33 (m, 1H), 6.90- 6.95 (m, 2H), 7.27-7.32 (m, 1H) | 466 | 464 | |

TABLE 220

| | | | | | Infor- |
|---|---|---|---|---|---|
| | | | | | mation |
| Exam- | | | MS | | of |
| ple | Chemical structure | NMR | M + H | M −H | structure |
| F-323 | | (400 MHz, CDCl3) 0.51-0.58 (m, 1H), 0.90 (m, 10H), 1.00-1.14 (m, 3H), 1.23-1.30 (m, 2H), 1.70- 1.81 (m, 2H), 2.04-2.22 (m, 2H), 2.33 (s, 3H), 2.52-2.62 (m, 3H), 2.65 (s, 3H), 2.75-2.83 (m, 1H), 3.18-3.26 (m, 1H), 3.81 (s, 3H), 4.06-4.14 (m, 1H), 4.23-4.31 (m, 1H), 5.09-5.15 (m, 1H), 6.20- 6.28 (m, 1H), 6.60 (brs, 1H), 6.62- 6.66 (m, 1H), 6.88-6.93 (m, 1H) | 496 | 494 | |
| F-324 | | (400 MHz, CDCl3) 0.90 (s, 9H), 0.94-1.03 (m, 2H), 1.18-1.35 (m, 4H), 1.72-1.85 (m, 2H), 2.09 (s, 3H), 2.14 (s, 3H), 2.25 (s, 3H), 2.70-2.84 (m, 2H), 2.95-3.04 (m, 1H), 3.26-3.35 (m, 1H), 3.66- 3.77 (m, 1H), 3.84-3.97 (m, 1H), 4.17-4.29 (m, 2H), 4.41-4.50 (m, 1H), 6.91-6.98 (m, 1H), 7.02- 7.08 (m, 1H), 7.60-7.66 (m, 1H), 9.05 (brs, 1H) | 466 | 464 | Racemic form |
| F-325 | | (400 MHz, CDCl3) 0.96-1.07 (m, 2H), 1.19-1.30 (m, 6H), 2.11- 2,22 (m, 7H), 2.24-2.30 (m, 4H), 3.01-3.06 (m, 1H), 3.08-3.14 (m, 2H), 3.27 (t, J = 11.02 Hz, 1H), 3.46-3.52 (m, 1H) 3.69-3.77 (m, 1H), 3.88-4.02 (m, 3H), 4.06-4.28 (m, 2H), 4.48 (dd, J = 11.48, 8.46 Hz, 1H), 6.94-6.99 (m, 2H), 7.76 (d, J = 8.12Hz, 1H), 8.99 (brs, 1H) | 522 | 520 | Racemic form |

TABLE 221

| | | | | | Infor- |
|---|---|---|---|---|---|
| | | | | | mation |
| Exam- | | | MS | | of |
| ple | Chemical structure | NMR | M + H | M − H | structure |
| F-326 | | (400 MHz, CDCl3) 0.87 (s, 9H), 0.93-0.98 (m, 1H), 1.08-1.19 (m, 3H), 1.24-1.28 (m, 2H), 1.68- 1.76 (m, 2H), 1.93-2.01 (m, 1H), 2.06-2.14 (m, 1H), 2.06 (s, 3H), 2.24 (s, 3H), 2.72 (dd, J = 8.06, 7.66 Hz, 2H), 2.86 (dd, J = 14.10, 4.84 Hz, 1H), 2.98-3.04 (m, 1H), 3.11 (dd, J = 14.10, 10.07 Hz, 1H), 3.66-3.79 (m, 2H), 3.96-4.03 (m, 1H), 6.91-6.93 (m, 2H), 7.38 (d, J = 8.06 Hz, 1H), 7.90 (s, 1H) | 427 | 425 | |
| F-327 | | (400 MHz, CDCl3) 0.87 (s, 9H), 0.92-0.97 (m, 1H), 1.08-1.18 (m, 3H), 1.24-1.28 (m, 2H), 1.68- 1.76 (m, 2H), 1.93-2.01 (m, 1H), 2.06 (s, 3H), 2.07-2.13 (m, 1H), 2.24 (s, 3H), 2.73 (dd, J = 8.46, 7.66 Hz, 2H), 2.88 (dd, J = 14.10, 4.43 Hz, 1H), 2.98-3.04 (m, 1H), 3.11 (dd, J = 11.28, 10.88 Hz, 1H), 3.66-3.66 (m, 2H), 3.95-4.03 (m, 1H), 6.91-6.93 (m, 2H), 7.37 (d, J = 7.66 Hz, 1H), 7.94 (s, 1H) | 427 | 425 | |
| F-328 | | (400 MHz, CDCl3) 1.11-1.51 (m, 13H), 2.14 (s, 3H), 2.17 (s, 3H), 2.23 (s, 3H), 2.42-2.56 (m, 2H), 2.77-2.92 (m, 2H), 2.97 (ddd, J = 14.34, 7.06, 3.53 Hz, 1H), 3.28 (t, J = 11.03 Hz, 1H), 3.70 (dd, J = 18.86, 10.46 Hz, 1H), 3.90 (dt, J = 20.88, 7.94 Hz, 1H), 4.20 (dt, J = 16.17, 6.62 Hz, 2H), 4.46 (dd, J = 11.47, 8.38 Hz, 1H), 6.95 (d, J = 10.37 Hz, 2H), 7.77 (dd, J = 7.61, 4.52 Hz, 1H), 9.05 (s, 1H) | 464 | 462 | Racemic form (Stereo- chemistry on Cyclopropane ring: Undetermined) |

TABLE 222

| | | | | | Infor- |
|---|---|---|---|---|---|
| | | | | | mation |
| Exam- | | | MS | | of |
| ple | Chemical structure | NMR | M + H | M − H | structure |
| F-329 | | (400 MHz, CDCl3) 0.76-1.36 (m, 4H), 1.16 (J = 6.00 Hz, 6H), 2.06 (s, 0.6H), 2.08 (s, 0.6H), 2.13 (s, 4.8H), 2.26 (s, 3H), 2.40-2.52 (m, 2H), 2.60-2.74 (m, 2H), 2.87- 2.97 (m, 1H), 3.11 (t, 10.92 Hz, 0.8H), 3.54-3.77 (m, 3H), 3.81- 3.98 (m, 1.2H), 4.15-4.48 (m, 4H), 6.89-7.01 (m, 2H), 7.55 (d, J = 7.88 Hz, 0.2H), 7.71 (d, J = 8.12 Hz, 0.8H), 8.11 (s, 0.2H), 8.92 (s, 0.8H) | 480 | 478 | Racemic form |
| F-330 | | (400 MHz, CDCl3) 0.78-1.07(m, 2H), 1.09-1.32 (m, 8H), 2.06 (s, 0.5H), 2.09 (s, 0.6H), 2.13 (s, 2.5H), 2.15 (s, 2.4H), 2.25 (s, 3H), 2.34 -2.51 (m, 2H), 2.65-2.79 (m, 2H), 2.87-2.98 (m, 1H), 3.04- 3.16 (m, 1H), 3.28 (t, J = 11.12 Hz, 0.8H), 3.54-3.73 (m, 2H), 3.84- 3.96 (m, 1.2H), 4.01-4.14 (m, 1.4H), 4.14-4.27 (m, 1.8H), 4.44 (dd, J = 11.12, 8.36 Hz, 0.8H), 6.91- 6.99 (m, 2H), 7.59 (d, J = 8.20 Hz, 0.2H), 7.74 (d, J = 5.12 Hz, 0.8 H), | 480 | 478 | Racemic form |
| | | 8.01 (s, 0.2H), 8.96 (s, 0.8H) | | | |
| F-331 | | (400 MHz, CDCl3) 0.90 (s, 9H), 0.94-1.03 (m, 2H), 1.19-1.35 (m, 4H), 1.73-1.83 (m, 2H), 2.14 (s, 3H), 2.18 (s, 3H), 2.28 (s, 3H), 2.70-2.84 (m, 2H), 2.96-3.04 (m, 1H), 3.25-3.35 (m, 1H), 3.64- 3.74 (m, 1H), 3.85-3.97 (m, 1H). 4.17-4.29 (m, 2H), 4.43-4.51 (m, 1H), 6.80-6.86 (m, 1H), 6.98- 7.03 (m, 1H), 7.78 (brs, 1H), 9.10 (brs, 1H) | 466 | 464 | Racemic form |

TABLE 223

| | | | | | Infor- |
|---|---|---|---|---|---|
| | | | | | mation |
| Exam- | | | MS | | of |
| ple | Chemical structure | NMR | M + H | M − H | structure |
| F-332 | | (400 MHz, CDCl3) 0.90 (s, 9H), 0.93-1.02 (m, 2H), 1.17-1.32 (m, 4H), 1.71-1.84 (m, 2H), 2.06 (s, 3H), 2.14 (s, 3H), 2.67-2.85 (m, 2H), 2.93-3.03 (m, 1H), 3.27- 3.35 (m, 1H), 3.66-3.76 (m, 1H), 3.79 (s, 3H), 3.88-3.97 (m, 1H), 4.17-4.28 (m, 2H), 4.42-4.49 (m, 1H), 6.62-6.68 (m, 1H), 7.08- 7.14 (m, 1H), 7.47-7.52 (m, 1H), 9.05 (brs, 1H) | 482 | 480 | Racemic form |
| F-333 | | (400 MHz, CDCl3) 0.84-0.93 (m, 11H), 0.93-1.03 (m, 2H), 1.18- 1.35 (m, 3H), 1.71-1.84 (m, 2H), 2.14 (s, 3H), 2.20 (s, 3H), 2.67- 2.85 (m, 1H), 2.94-3.04 (m, 1H), 3.25-3.35 (m, 1H), 3.63-3.75 (m, 1H), 3.85-4.13 (m, 1H), 4.14- 4.28 (m, 2H), 4.44-4.52 (m, 1H), 6.81-6.88 (m, 2H), 7.79-7.86 (m, 1H), 9.25 (brs, 1H) | 470 | 468 | Racemic form |
| F-334 | | (400 MHz, CDCl3) 0.76-1.36 (m, 4H), 1.16 (J = 6.00 Hz, 6H), 2.06 (s, 0.6H), 2.08 (s, 0.6H), 2.13 (s, 4.8H), 2.26 (s, 3H), 2.40-2.52 (m, 2H), 2.60-2.74 (m, 2H), 2.87- 2.97 (m, 1H), 3.11 (t, J = 10.88 Hz, 0.8H), 3.54-3.77 (m, 3H), 3.81- 3.98 (m, 1.2H), 4.15-4.28 (m, 2H) 4.34 -4.48 (m, 2H), 6.89-7.01 (m, 2H),7.55 (d, J = 7.88 Hz, 0.2H), 7.71 (d, J = 6.12 Hz, 0.8H), 8.11 (s, 0.2H), 8.92 (s, 0.8H) | 480 | 478 | |

TABLE 224

| | | | | | Infor- |
|---|---|---|---|---|---|
| | | | | | mation |
| Exam- | | | MS | | of |
| ple | Chemical structure | NMR | M + H | M − H | structure |
| F-335 | | (400 MHz, CDCl3) 0.76-1.36 (m, 4H), 1.16 (J = 6.00 Hz, 6H), 2.06 (s, 0.6H), 2.08 (s, 0.6H), 2.13 (s, 4.8H), 2.26 (s, 3H), 2.40-2.52 (m, 2H), 2.60-2.74 (m, 2H), 2.87- 2.97 (m, 1H), 3.11 (t, J = 10.88 Hz, 0.8H), 3.54-3.77 (m, 3H), 3.81- 3.98 (m, 1.2H), 4.15-4.28 (m, 2H), 4.34-4.48 (m, 2H), 6.89-7.01 (m, 2H), 7.55 (d, J = 7.68 Hz, 0.2H), 7.71 (d, J = 8.12 Hz, 0.8H), 8.11 (s, 0.2H), 8.92 (s, 0.8H) | 480 | 478 | |
| F-336 | | (400 MHz, CDCl3) 0.78-1.07 (m, 2H), 1.09-1.32 (m, 8H) 2.06 (s, 0.5H), 2.09 (s, 0.6H), 2.13 (s, 2.5H), 2.15 (s, 2.4H), 2.25 (s, 3H), 2.34-2.51 (m, 2H), 2.65-2.79 (m, 2H), 2.87-2.98 (m, 1H), 3.04- 3.16 (m, 1H), 3.28 (t, J = 11.12 Hz, 0.8H), 3.54-3.73 (m, 2H), 3.84- 3.96 (m, 1.2H), 4.01-4.14 (m, 1.4H), 4.14-4.27 (m, 1.8H), 4.44 (dd, J = 11.12, 8.36 Hz, 0.8H), 6.91-6.99 (m, 2H), 7.59 (d, J = 8.20 Hz, 0.2H), 7.74 (d, J = 8.12 | 480 | 478 | |
| | | Hz, 0.8 H), 8.01 (s, 0.2H), 8.96 (s, | | | |
| | | 0.8H) | | | |

TABLE 225

| | | | | | Informa- |
|---|---|---|---|---|---|
| | | | MS | | tion of |
| Example | Chemical structure | NMR | M + H | M − H | structure |
| F-337 | | (400 MHz, CDCl3) 0.78-1.07 (m, 2H), 1.09-1.32 (m, 8H), 2.06 (s, 0.5H), 2.09 (s, 0.6H), 2.13 (s, 2.5H), 2.15 (s, 2.4H), 2.25 (s, 3H), 2.34-2.51 (m, 2H), 2.65-2.79 (m, 2H), 2.87-2.98 (m, 1H), 3.04- 3.16 (m, 1H), 3.28 (t, J = 11.12Hz, 0.8H), 3.54-3.73 (m, 2H), 3.84- 3.96 (m, 1.2H), 4.01-4.14 (m, 1.4H), 4.14-4.27 (m, 1.8H), 4.44 (dd, J = 11.12, 8.36Hz, 0.8H), 6.91- 6.99 (m, 2H), 7.59 (d, J = 8.20Hz, 0.2H), 7.74 (d, J = 8.12Hz, 0.8H), 8.01 (s, 0.2H), 8.96 (s, 0.8H) | 480 | 478 | |
| F-338 | | (400 MHz, CDCl3) 0.89 (d, J = 1.98Hz, 9H), 0.99 (t, J = 5.40Hz, 2H), 1.23-1.28 (m, 4H), 1.74- 1.78 (m, 2H), 2.13 (s, 3H), 2.16 (s, 3H), 2.25 (s, 3H), 2.77 (td, J = 7.77, 4.26Hz, 2H), 2.99 (t, J = 3.86Hz. 1H), 3.30 (t, J = 11.03Hz, 1H), 3.71 (d, J = 8.38Hz, 1H), 3.92 (s, 1H), 4.20-4.24 (m, 2H), 4.45 (dd, J = 11.36, 8.49Hz, 1H), 6.95 (d, J = 10.14Hz, 2H), 7.76 (d, J = 7.94Hz, 1H), 9.05 (s, 1H) | 466 | 464 | |
| F-339 | | (400 MHz, CDCl3) 0.89 (s, 9H), 0.96-0.98 (m, 2H), 1.24-1.28 (m, 4H), 1.73-1.77 (m, 2H), 2.13 (s, 3H), 2.16 (s, 3H), 2.25 (s, 3H), 2.76 (td, J = 7.77, 4.04Hz, 2H), 2.99 (t, J = 3.86Hz, 1H), 3.30 (t, J = 11.03Hz, 1H), 3.71 (d, J = 9.26Hz, 1H), 3.90-3.94 (m, 1H), 4.22 (t, J = 6.29Hz, 2H), 4.43- 4.46 (m, 1H), 6.94-6.96 (m, 2H), 7.75 (d, J = 7.94Hz, 1H), 9.06 (s, 1H) | 466 | 464 | |

TABLE 226

| Ex- | | | | | Informa- |
|---|---|---|---|---|---|
| am- | | | MS | | tion of |
| ple | Chemical structure | NMR | M + H | M − H | structure |
| F-340 | | (400 MHz, CDCl3) 0.64-1.50 (m, 8H), 0.96 (d, J = 6.84Hz, 6H), 1.59- 1.82 (m, 2H), 2.11 (s, 3H), 2.13 (s, 3H), 2.25 (s, 3H), 3.04 (tt, J = 7.12, 3.96Hz, 1H), 3.26-3.35 (m, 1H), 3.64-3.74 (m, 1H), 3.86-4.28 (m, 4H), 4.46 (dd, J = 11.47, 8.60Hz, 1H), 6.93 (s, 1H), 6.95 (d, J = 7.94Hz, 1H), 7.73 (d, J = 7.94Hz, 1H), 8.97 (brs, 1H) | 464 | 462 | Racemic form (Stereo- chemistry or Pyrrolidine ring and Cyclopropyl ring: Trans isomer, both) (Diastereomer of F-341) |
| F-341 | | (400 MHz. CDCl3) 0.84-1.57 (m, 8H), 0.95 (d, J = 6.62Hz, 6H), 1.62- 1.81 (m, 2H), 2.11 (s, 3H), 2.13 (s, 3H), 2.25 (s, 3H), 3.04 (tt, J = 7.12, 3.96Hz, 1H), 3.24-3.34 (m, 1H), 3.64-3.75 (m, 1H), 3.83-4.28 (m, 4H), 4.45 (dd, J = 11.36, 8.49Hz, 1H), 6.94 (s, 1H), 6.96 (d, J = 8.16Hz, 1H), 7.73 (d, J = 8.16Hz, 1H), 8.92 (brs, 1H) | 464 | 462 | Racemic form (Stereo- chemistry or Pyrrolidine ring and Cyclopropyl ring: Trans isomer, both) (Diastereomer of F-340) |
| F-342 | | (400 MHz. CDCl3) 0.87 (d, J = 6.62Hz, 6H), 0.92-0.98 (m, 1H), 1.09-1.30 (m, 5H), 1.50- 1.61 (m, 1H), 1.68-1.83 (m, 3H), 1.90-1.98 (m, 1H), 2.07 (s, 3H), 2.24 (s, 3H), 2.69-2.76 (m, 2H), 2.94-3.04 (m, 2H), 3.11 (dd, J = 14.45, 9.15Hz, 1H), 3.46 (dd, J = 11.25, 7.06Hz, 1H), 3.58 (dd, J = 11.14, 3.42Hz, 1H), 3.84-3.91 (m, 1H), 3.98-4.06 (m, 1H), 6.89- 6.95 (m, 2H), 7.34 (d, J = 7.94Hz, 1H), 8.27 (brs, 1H) | 443 | 441 | |

TABLE 227

| Ex- | | | | | Informa- |
|---|---|---|---|---|---|
| am- | | | MS | | tion of |
| ple | Chemical structure | NMR | M + H | M − H | structure |
| F-343 | | (400 MHz, CDCl3) 0.87 (d, J = 6.62Hz, 6H), 1.00-1.30 (m, 6H), 1.50-1.60 (m, 1H), 1.66- 1.77 (m, 2H), 1.86-1.96 (m, 1H), 1.97-2.06 (m, 1H), 2.04 (s, 3H), 2.24 (s, 3H), 2.68-2.76 (m, 2H), 2.87 (dd, J = 14.11, 5.07Hz, 1H), 2.99-3.08 (m, 2H), 3.44 (dd, J = 10.92, 6.73Hz, 1H), 3.59-3.70 (m, 2H), 4.02-4.11 (m, 1H), 6.89- 6.96 (m, 2H), 7.34 (d, J = 7.72Hz, 1H), 7.83 (brs, 1H) | 443 | 441 | |
| F-344 | | (400 MHz, MeOH-D4) 0.90 (s, 9H), 1.07-1.23 (m, 4H), 1.25-1.32 (m, 2H), 1.71-1.82 (m, 2H), 1.96 (s, 3H), 2.07 (s, 3H), 2.24 (s, 3H), 2.82 (t, J = 7.83Hz, 2H), 3.03 (dd, J = 15.00, 5.95Hz, 1H), 3.10-3.17 (m, 1H), 3.26 (dd, J = 15.00, 9.48Hz, 1H), 5.89 (dd, J = 9.48, 5.95Hz, 1H), 6.92 (d, J = 8.16Hz, 1H), 6.98 (s, 1H), 7.06 (d, J = 8.16Hz, 1H) | 440 | 438 | |
| F-345 | | (400 MHz, CDCl3) 0.83-1.01 (m, 2H), 1.14-1.24 (m, 2H), 2.07 (s, 0.6H), 2.12 (s, 0.6H), 2.14 (s, 2.6H), 2.18 (s, 2.2H), 2.26 (s, 3H), 2.59-2.73 (m, 2H), 2.79-3.00 (m, 3H), 3.34 (t, J = 11.12Hz, 0.8H), 3.65-3.78 (m, 2.2H), 3.85-4.00 (m, 2H), 4.05-4.13 (m, 0.4H), 4.18- 4.30 (m, 1.8H), 4.46 (dd, J = 11.60, 8.56Hz, 0.8H), 6.92- 7.00 (m, 2H), 7.18-7.40 (m, 5H), 7.60 (d, J = 7.88Hz, 0.2H), 7.76 (d, J = 8.12Hz, 0.8H), 8.09 (s, 0.2H), 8.98 (s, 0.8H) | 498 | 496 | Racemic form |

TABLE 228

| Ex- | | | | | Informa- |
|---|---|---|---|---|---|
| am- | | | MS | | tion of |
| ple | Chemical structure | NMR | M + H | M − H | structure |
| F- 346 | | (400 MHz, DMSO-D6) 0.89-1.05 (m, 2H), 1.06-1.20 (m, 2H), 1.97 (s, 1.5H), 2.00 (s, 1.5H), 2.03 (s, 3H), 2.22 (s, 3H), 2.31-2.55 (m, 2H), 2.71-2.86 (m, 2H), 3.03- 3.14 (m, 1H), 3.28-3.50 (m, 1H), 3.53-3.65 (m, 1.5H), 3.65-3.78 (m, 2H), 3.78-3.88 (m, 0.5H), 3.89- 4.15 (m, 3H), 6.93 (d, J = 8.24Hz, 1H), 6.98 (s, 1H), 7.16-7.38 (m, 6H), 9.52 (d, J = 8.68Hz, 1H) | 498 | 496 | Racemic form |
| F- 347 | | (400 MHz, DMSO-D6) 0.90-1.18 (m, 10H), 1.21-1.37 (m, 0.3H), 1.48-1.59 (m, 1.7H), 1.65-1.75 (m, 0.3H), 1.82-1.92 (m, 1.7H), 1.97 (s, 1.5H), 2.00 (s, 1.5H), 2.02- 2.06 (m, 3H), 2.22 (s, 3H), 2.65- 2.77 (m, 12H), 2.78-2.89 (m, 1.8H), 3.03-3.13 (m, 1H), 3.28- 3.38 (m, 0.5H), 3.40-3.48 (m, 0.5H), 3.51-3.61 (m, 0.5H), 3.64- 3.75 (m, 1H), 3.76-3.85 (m, 0.5H), 3.88-4.14 (m, 3H), 6.93 (d, J = 8.00Hz, 1H), 6.99 (s, 1H), 7.15- 7.25 (m, 1H), 9.47-9.59 (m, 1H) | 464 | 462 | Racemic form |
| F- 348 | | (400 MHz, CDCl3) 0.90 (t, J = 5.62Hz, 9H), 0.93-1.49 (m, 5H), 1.95-2.01 (m, 3H), 2.12 (t, J = 4.85Hz, 6H), 2.27 (d, J = 9.26Hz, 3H), 2.87 (d, J = 8.60Hz, 1H), 3.02 (dt, J = 12.79, 4.47Hz, 1H), 3.29 (t, J = 11.03Hz, 1H), 3.83 (dt, J = 20.51, 7.28Hz, 1H), 3.89-3.98 (m, 1H), 4.16 (ddd, J = 19.02, 11.63, 5.57Hz, 2H), 4.42 (dd, J = 11.47, 8.38Hz, 1H), 4.85 (d, J = 5.95Hz, 1H), 6.95 (d, J = 9.92Hz, 2H), 7.66 (d, J = 7.94Hz, 1H), 8.88 (s, 1H) | 482 | 480 | Racemic form (Stereo- chemistry of Hydroxyl group: Undeter- mined) (Dia- stereomer of F-349) |

TABLE 229

| Ex- | | | | | Informa- |
|---|---|---|---|---|---|
| am- | | | MS | | tion of |
| ple | Chemical structure | NMR | M + H | M − H | structure |
| F-349 | | (400 MHz, CDCl3) 0.90 (d, J = 2.65Hz, 9H), 0.96-1.48 (m, 5H), 1.97-2.03 (m, 1H), 2.11- 2.13 (m, 6H), 2.25 (s, 3H), 3.09 (dd, J = 13.12, 8.93Hz, 1H), 3.19- 3.25 (m, 1H), 3.61 (dd, J = 18.08, 8.16Hz, 1H), 3.75-3.83 (m, 1H), 3.96 (dt, J = 15.22, 4.63Hz, 1H), 4.10 (dt, J = 12.06, 5.68Hz, 2H), 4.33 (dd, J = 11.47, 8.60Hz, 1H), 4.83 (dd, J = 13.56, 7.39Hz, 1H), 6.96 (d, J = 8.16Hz, 2H), 7.63 (d, J = 7.94Hz, 1H), 8.85 (s, 1H) | 482 | 480 | Racemic form (Stereo- chemistry of Hydroxyl group: Undeter- mined) (Dia- stereomer of F-348) |
| F-350 | | (400 MHz, CDCl3) 0.77-0.94 (m, 1H), 0.95-1.11 (m, 1H), 1.20- 1.44 (m, 2H), 1.38 (d, J = 6.92Hz, 6H), 2.08 (s, 0.7H), 2.12 (s, 0.7H), 2.15 (s, 2.3H), 2.18 (s, 2.3H), 2.26 (s, 3H), 3.11-3.22 (m, 1H), 3.27- 3.43 (m, 1.8H), 3.60-3.69 (m, 0.2H), 3.78-4.02 (m, 2H), 4.12- 4.31 (m, 2.2H), 4.53 (dd, J = 11.56, 8.44Hz, 0.8H), 6.57 (s, 1H), 6.95 (s, 1H), 6.97 (d, J = 8.48Hz, 1H), 7.59 (d, J = 7.80Hz, 0.2H), 7.74 (d, J = 8.24Hz, 0.8H), 7.95 (s, 0.2H), 8.77 (s, 0.8H) | 477 | 475 | Racemic form |

TABLE 230

| Ex- | | | | | Informa- |
|---|---|---|---|---|---|
| am- | | | MS | | tion of |
| ple | Chemical structure | NMR | M + H | M − H | structure |
| F-351 | | (400 MHz, CDCl3) 0.89 (s, 9H), 1.21-1.39 (m, 2H), 1.33 (t, J = 7.36Hz, 3H), 1.71-1.84 (m, 2H), 2.05-2.07 (m, 1.2H), 2.09 (s, 2.4H), 2.13 (s, 2.4H), 2.25 (s, 3H), 2.67 (dd, J = 15.60, 7.36Hz, 2H), 3.27 (t, J = 11.12Hz, 0.8H), 3.47- 3.59 (m, 0.8H), 3.79-3.96 (m, 3.4H), 3.96-4.06 (m, 0.4H), 4.15- 4.33 (m, 1.8H), 4.37 (dd, J = 11.60, 8.24Hz, 0.8H), 6.91-6.99 (m, 2H), 7.53 (d, J = 8.92Hz, 0.2H), 7.69 (d, J = 8.04Hz, 0.8H), 7.72 (s, 0.2H), 8.62 (s, 0.8H) | 454 | 452 | Racemic form |
| F-352 | | (400 MHz, DMSO-D6) 0.61-1.08 (m, 4H), 0.86 (s, 9H), 1.17-1.27 (m, 2H), 1.61-1.74 (m, 2H), 2.06 (s, 3H), 2.22 (s, 3H), 2.66-2.76 (m, 5H), 2.80-2.99 (m, 2H), 3.27- 3.36 (m, 1H), 5.03-5.19 (m, 2H), 6.01-6.19 (m, 1H), 6.91 (d, J = 8.60Hz, 1H), 6.97 (s, 1H), 7.14 (d, J = 8.16Hz, 1H), 7.28-7.45 (m, 5H), 9.36-9.44 (m, 1H) | 546 | 544 | |
| F-353 | | (400 MHz, MeOH-D4) 0.90 (s, 9H), 1.02-1.10 (m, 1H), 1.19-1.32 (m, 5H), 1.72-1.83 (m, 2H), 2.05 (s, 3H), 2.25 (s, 3H), 2.38 (s, 3H), 2.84 (t, J = 7.72Hz, 2H), 3.04-3.09 (m, 2H), 3.16-3.22 (m, 1H), 4.63 (t, J = 7.39Hz, 1H), 6.93 (d, J = 7.50Hz, 1H), 6.98(8, 1H), 7.10 (d, J = 8.16Hz, 1H) | 412 | 410 | |

TABLE 231

| | | | | | Infor- |
|---|---|---|---|---|---|
| | | | | | ma- |
| Ex- | | | | | tion of |
| am- | | | MS | | struc- |
| ple | Chemical structure | NMR | M + H | M − H | ture |
| F-354 | | (400 MHz, DMSO- D6) 0.80-1.30 (m, 6H), 0.87 (s, 9H), 1.61-1.74 (m, 2H), 2.00-2.29 (m, 9H), 2.60- 3.34 (m, 8H), 5.75 and 6.47 (t and dd, J = 7.72 and 9.37, 5.84Hz, total 1H), 6.88-7.01 (m, 2H), 7.10- 7.17 (m, 1H), 9.33 | 454 | 452 | |
| | | and 9.48 (each | | | |
| | | s, total 1H) | | | |
| F-355 | | (400 MHz, MeOH- D4) 0.90 (s, 9H), 1.06-1.32 (m, 6H), 1.70-1.81 (m, 2H), 2.07 (s, 3H), 2.25 (s, 3H), 2.77-2.86 (m, 2H), 3.00-3.14 (m, 2H), 3.23-3.35 (m, 1H), 5.08 (s, 2H), 5.65 (t, J = 7.39Hz, 1H), 6.93 (d, J = 8.16Hz, 1H), 6.99 (s, 1H), 7.07 (d, J = 8.16Hz, 1H), 7.25-7.35 | 532 | 530 | |
| | | (m, 5H) | | | |
| F-356 | | (400 MHz, DMSO- D6) 0.87 (s, 9H), 1.21-1.31 (m, 2H), 1.63- 1.75 (m, 2H), 1.94-2.03 (m, 6H), 2.22 (s, 3H), 2.62- 2.72 (m, 2H), 3.37-3.46 (m, 0.5H), 3.48-3.58 (m, 0.7H), 3.62- 3.73 (m, 1H), 3.75-3.90 (m, 1H), 3.90-4.15 (m, | 508 | 506 | Race- mic form |
| | | 2.8H), 5.00-5.24 | | | |
| | | (m, 2H), 6.93 (d, | | | |
| | | J = 8.24Hz, 1H), | | | |
| | | 6.99 (s, 1H), 7.11- | | | |
| | | 7.20 (m, 1H), 9.43- | | | |
| | | 9.52 (m, 1H) | | | |
| F-357 | | (400 MHz, CDCl3) 0.83-0.91 (m, 2H), 0.87 (d, J = 6.85Hz, 6H), 1.08- 1.15 (m, 2H), 1.43 (dd, J = 6.80, 7.20Hz. 2H), 1.50- 1.63 (m, 1H), 2.00-2.12 (m, 2H), 2.19 (s, 3H), | 395 | 393 | |
| | | 2.26 (s, 3H), | | | |
| | | 2.48-2.63 (m, 3H), | | | |
| | | 2.88 (tt, J = 7.20, | | | |
| | | 4.00Hz, 1H), 3.05 | | | |
| | | (t, J = 6.35Hz, | | | |
| | | 2H), 3.16 (t, | | | |
| | | J = 6.35Hz, 2H), | | | |
| | | 3.55-3.66 (m, | | | |
| | | 1H), 6.92-6.99 | | | |
| | | (m, 2H), 7.60 (d, | | | |
| | | J = 8.87Hz, 1H), | | | |
| | | 8.30 (brs, 1H) | | | |

TABLE 232

| | | | | | Infor- |
|---|---|---|---|---|---|
| | | | | | ma- |
| Ex- | | | | | tion of |
| am- | | | MS | | struc- |
| ple | Chemical structure | NMR | M + H | M − H | ture |
| F- 358 | | (400 MHz, CDCl3) 0.86 (d, J = 6.62Hz, 6H), 0.87-0.93 (m, 2H), 1.09-1.17 (m, 2H), 1.34 (dd, J = 7.12, 6.73Hz, 2H), 1.49-1.60 (m, 1H), 2.01-2.12 (m, 2H), 2.19 (s, 3H), 2.26 (s, | 395 | 393 | |
| | | 3H), 2.34-2.59 | | | |
| | | (m, 3H), 2.89 (tt, | | | |
| | | J = 7.12, 4.35Hz, | | | |
| | | 1H), 3.00-3.09 | | | |
| | | (m, 2H), 3.11- | | | |
| | | 3.19 (m, 2H), 3.43 | | | |
| | | (tt, J = 9.50, | | | |
| | | 7.92Hz, 1H), 6.93- | | | |
| | | 7.00 (m, 2H), | | | |
| | | 7.60 (d, J = 8.60Hz, | | | |
| | | 1H), 8.31 (s, 1H) | | | |
| F- 359 | | (400 MHz, MeOH- D4) 0.90 (s, 9H), 1.22 (d, J = 96.38Hz, 6H), 1.77 (d, J = 39.04Hz, 2H), 2.07 (s, 3H), 2.25 (s, 3H), 2.83 (t, J = 7.83Hz, 2H), 2.99 (dd, J = 15.11, 6.73Hz, 1H), 3.10-3.24 (m, 2H), 4.78 (t, J = 7.83Hz, 1H), 6.93 (d, J = 7.50Hz, | 398 | 396 | |
| | | 1H), 6.99 (s, 1H), 7.09 (d, | | | |
| | | J = 7.94Hz, 1H) | | | |
| F- 360 | | (400 MHz, CDCl3) 0.85 (d, J = 6.72Hz, 6H), 0.87-0.93 (m, 1H), 0.99-1.05 (m, 1H), 1.08- 1.17 (m, 2H), 1.28-1.35 (m, 2H), 1.48-1.58 (m, 1H), 1.91-2.14 (m, 4H), 2.07 (s, 3H), 2.25 (s, 3H), 2.31-2.52 (m, 3H), 2.83-2.96 (m, | 439 | 437 | |
| | | 2H), 3.10 (dd, J = | | | |
| | | 14.03, 9.86Hz, | | | |
| | | 1H), 3.36-346 | | | |
| | | (m, 1H), 3.67- | | | |
| | | 3.79 (m, 2H), 3.91- | | | |
| | | 3.99 (m, 1H), | | | |
| | | 6.90-6.97 (m, | | | |
| | | 2H), 7.39 (d, | | | |
| | | J = 8.12Hz, 1H), | | | |
| | | 7.76 (brs, 1H) | | | |

TABLE 233

| Ex- | | | | | Informa |
|---|---|---|---|---|---|
| am- | | | MS | | tion of |
| ple | Chemical structure | NMR | M + H | M − H | structure |
| F-361 | | (400 MHz, CDCl3) 0.85 (d, J = 6.72Hz, 6H), 0.87-0.93 (m, 1H), 0.99-1.05 (m, 1H), 1.08- 1.17 (m, 2H), 1.28-1.35 (m, 2H), 1.48-1.58 (m, 1H), 1.91-2.14 (m, 4H), 2.07 (s, 3H), 2.25 (s, 3H), 2.31-2.52 (m, 3H), 2.83-2.96 (m, 2H), 3.10 (dd, J = 14.03, 9.86Hz, 1H), 3.36-3.46 (m, 1H), 3.67- 3.79 (m, 2H), 3.91-3.99 (m, 1H), 6.90-697 (m, 2H), 7.39 (d, J = 8.12Hz, 1H), 7.76 (brs, 1H) | 439 | 437 | |
| F-362 | | (400 MHz, CDCl3) 0.79-0.83 (m, 1H), 0.87 (d, J = 7.05Hz, 6H), 0.91- 1.01 (m, 3H), 1.19-1.23 (m, 2H), 1.26 (d, J = 6.04Hz, 3H), 1.49-1.59 (m, 1H), 1.61-1.69 (m, 2H), 2.04 (s, 3H), 2.06-2.15 (m, 2H), 2.24 (s, 3H), 2.54-2.68 (m, 2H), 2.75- 2.81 (m, 2H), 3.03 (dd, J = 13.50, 10.68Hz, 1H), 3.49-3.56 (m, 1H), 3.90-3.97 (m, 1H), 4.15 (d, J = 11.69Hz, 1H), 4.49 (d, J = 11.69Hz, 1H), 6.91-6.92 (m, 2H), 7.26-7.22 (m, 3H), 7.28- 7.32 (m, 2H), 7.41 (d, J = 5.64Hz, 1H), 7.54 (s, 1H) | 517 | 515 | |

TABLE 234

| Ex- | | | | | Informa- |
|---|---|---|---|---|---|
| am- | | | MS | | tion of |
| ple | Chemical structure | NMR | M + H | M − H | structure |
| F-363 | | (400 MHz, CDCl3) 0.84-0.91 (m, 1H), 0.87 (d, J = 6.45Hz, 6H), 0.95- 1.07 (m, 3H), 1.21 (d, J = 5.64Hz, 3H), 1.23-1.28 (m, 2H), 1.51- 1.60 (m, 1H), 1.68-1.77 (m, 2H), 1.79-1.86 (m, 1H), 1.92-1.99 (m, 1H), 2.07 (s, 3H), 2.24 (s, 3H), 2.70 (dd, J = 7.86, 7.66Hz, 2H), 2.83-2.90 (m, 2H), 2.98 (dd, J = 13.90, 10.68Hz, 1H), 3.74-3.81 (m, 1H), 3.96-4.03 (m, 1H), 4.47 (d, J = 11.28Hz, 1H), 4.64 (d, J = 11.69Hz, 1H), 6.91-6.93 (m, 2H), 7.25-7.28 (m, 1H), 7.32- 7.38 (m, 4H), 7.47 (d, J = 8.46Hz, 1H), 7.98 (s, 1H) | 517 | 515 | |
| F-364 | | (400 MHz, CDCl3) 0.86-0.90 (m, 7H), 0.92-0.97 (m, 1H), 1.07- 1.29 (m, 10H), 1.51-1.61 (m, 1H), 1.71-1.79 (m, 1H), 2.09 (s, 3H), 2.25 (s, 3H), 2.71-2.80 (m, 2H), 2.93-3.02 (m, 2H), 3.12 (dd, J = 14.31, 9.47Hz, 1H), 3.93-4.01 (m, 2H), 6.92-6.94 (m, 2H), 7.40 (d, J = 8.06Hz, 1H), 8.04 (s, 1H) | 427 | 425 | |
| F-365 | | (400 MHz. CDCl3) 0.87 (d, J = 6.45Hz, 6H), 0.98-1.03 (m, 1H), 1.06-1.28 (m, 8H), 1.51- 1.89 (m, 4H), 2.01-2.03 (m, 1H), 2.05 (s, 3H), 2.24 (s, 3H), 2.73 (dd, J = 8.06, 8.46Hz, 2H), 2.83 (dd, J = 13.90, 4.63Hz, 1H), 3.00-3.09 (m, 2H), 3.72-3.77 (m, 1H), 3.99- 4.06 (m, 1H), 6.92-6.93 (m, 2H), 7.40 (d, J = 8.87Hz, 1H), 7.59 (s, 1H) | 427 | 425 | |

TABLE 235

| Ex- | | | | | Informa- |
|---|---|---|---|---|---|
| am- | | | MS | | tion of |
| ple | Chemical structure | NMR | M + H | M − H | structure |
| F-366 | | (400 MHz, CDCl3) 0.94 (s, 9H), 1.19-1.32 (m, 4H), 1.49-1.53 (m, 2H), 1.94-2.05 (m, 1H), 2.03 (s, 3H), 2.07-2.15 (m, 1H), 2.25 (s, 3H), 2.41-2.54 (m, 2H), 2.91 (dd, J = 14.51, 4.84Hz, 1H), 3.11-3.21 (m, 2H), 3.65-3.79 (m, 2H), 4.05- 4.13 (m, 1H), 6.93-5.95 (m, 2H), 7.32 (d, J = 8.87Hz, 1H),7.52 (s, 1H) | 463 | 461 | Racemic form |
| F-367 | | (400 MHz, CDCl3) 0.89 (s, 9H), 1.00 (ddd, J = 10.53, 5.57, 3.03Hz, 1H), 1.14-1.25 (m, 5H), 1.42 (td, J = 12.79, 4.70Hz, 1H), 1.92-2.06 (m, 2H), 2.18 (s, 3H), 2.26 (s, 3H), 2.68 (d, J = 7.94Hz, 1H), 3.03 (dt, J = 12.57, 5.35Hz, 3H), 3.14-3.21 (m, 2H), 4.82 (dd, J = 13.34, 7.61Hz, 1H), 6.97 (t, J = 7.94Hz, 2H), 7.58 (t, J = 4.41Hz, 1H), 8.06 (s, 1H) | 399 | 397 | Racemic form |
| F-368 | | (400 MHz, CDCl3) 0.76-0.92 (m, 1H), 0.92-1.04 (m, 1H), 1.15- 1.35 (m, 2H), 1.56 (d, J = 6.92Hz, 6H), 2.08 (m, 0.6H), 2.11 (s, 0.6H), 2.14 (s, 2.4H), 2.19 (s, 2.4H), 2.26 (s, 3H), 3.16-3.27 (m, 1H), 3.36 (t, J = 10.92Hz, 0.8H), 3.67 (t, J = 9.84Hz, 0.2H), 3.72-3.84 (m, 0.8H), 3.84-4.01 (m, 1.2H), 4.07- 4.31 (m, 2.2H), 4.46-4.64 (m, 1.8H), 6.92-7.00 (m, 2H), 7.57 (d, J = 8.04Hz, 0.2H), 7.76 (d, J = 8.04Hz, 0.8H), 7.90 (s, 0.2H), 7.92 (s, 0.8H), 7.97 (s, 1H), 8.11 (s, 0.2H), 8.96 (s, 0.8H) | 476 | 474 | Racemic form |

TABLE 236

| Ex- | | | | | Informa- |
|---|---|---|---|---|---|
| am- | | | MS | | tion of |
| ple | Chemical structure | NMR | M + H | M − H | structure |
| F-369 | | (400 MHz, CDCl3) 0.82 (s, 9H) 1.20-1.24 (m, 2H), 1.30 (t, J = 7.54Hz, 3H), 1.61-1.69 (m, 2H), 2.04 (s, 3H), 2.09 (s, 3H), 2.27 (s, 3H), 2.36-2.43 (m, 1H), 2.56 (ddd, J = 31.25, 15.48, 8.06Hz, 2H), 2.71-2.78 (m, 1H), 3.82- 4.05 (m, 4H), 4.55 (td, J = 14.73, 7.27Hz, 1H), 5.30 (d, J = 6.72Hz, 1H), 6.92-7.01 (m, 2H), 7.41 (d, J = 8.81Hz, 1H), 8.09 (brs, 1H) | 454 | 452 | |
| F-370 | | (400 MHz, CDCl3) 0.90 (s, 9H), 1.18 (t, J = 7.42Hz, 3H), 1.28-1.33 (m, 2H), 1.72-1.80 (m, 2H), 2.00 (s, 3H), 2.22-2.31 (m, 1H), 2.33 (s, 3H), 2.44-2.51 (m, 1H), 2.59 (t, J = 8.00Hz, 2H), 3.46-3.52 (m, 1H), 3.74-3.89 (m, 3H), 3.80 (s, 3H), 4.32 (d, J = 6.03Hz, 2H), 4.45 (td, J = 14.78, 7.34Hz, 1H), 5.12 (d, J = 7.19Hz, 1H), 6.12-6.14 (brm, 1H), 6.64-6.70 (m, 2H), 7.03 (d, J = 7.42Hz, 1H) | 484 | 482 | |
| F-371 | | (400 MHz, CDCl3) 0.53-0.60 (m, 1H), 0.77-0.84 (m, 1H), 1.00- 1.07 (m, 1H), 1.11-1.20 (m, 1H), 1.29 (s, 3H), 1.31 (s, 3H), 2.16 (s, 3H), 2.20 (s, 3H), 2.27 (s, 3H), 2.95-3.02 (m, 1H), 3.23-3.28 (m, 1H), 3.43 (t, J = 10.90Hz, 1H), 3.81- 3.86 (m, 1H), 3.92-4.00 (m, 1H), 4.24-4.29 (m, 2H), 4.54 (dd, J = 11.36, 8.81Hz, 1H), 6.96-7.00 (m, 2H), 7.33-7.37 (m, 2H), 7.62 (d, J = 8.12Hz, 2H), 7.78 (d, J = 7.88Hz, 1H), 8.99 (brs, 1H) | 486 | 484 | |

TABLE 237

| Ex- | | | | | Informa- |
|---|---|---|---|---|---|
| am- | | | MS | | tion of |
| ple | Chemical structure | NMR | M + H | M − H | structure |
| F-372 | | (400 MHz, CDCl3) 0.52-0.61 (m, 1H), 0.77-0.84 (m, 1H), 1.00- 1.20 (m, 2H), 1.29 (s, 3H), 1.31 (s, 3H), 2.16 (s, 3H), 2.20 (s, 3H), 2.27 (s, 3H), 2.95-3.02 (m, 1H), 3.23-3.28 (m, 1H), 3.43 (t, J = 11.02Hz, 1H), 3.49 (d, J = 4.41Hz, 1H), 3.83 (dd, J = 18.90, 10.55Hz, 1H), 3.91-4.00 (m, 1H), 4.20-4.31 (m, 2H), 4.54 (dd, J = 11.36, 8.58Hz, 1H), 6.94-7.00 (m, 2H), 7.32-7.36 (m, 2H), 7.59- 7.65 (m, 2H), 7.77 (d, J = 7.88Hz, 1H), 9.00 (brs, 1H) | 486 | 484 | |
| F-373 | | (400 MHz, CDCl3) 0.88 (s, 9H), 1.28 (t, J = 8.49Hz, 2H), 1.76 (dd, J = 15.66, 7.94Hz, 2H), 2.02 (s, 3H), 2.25 (s, 3H), 2.49-2.71 (m, 4H), 2.86-2.99 (m, 2H), 3.35-3.42 (m, 1H), 4.21-4.29 (m, 1H), 5.03- 5.17 (m, 3H), 5.75-5.83 (m, 1H), 6.91-6.97 (m, 2H), 7.06 (brs, 1H), 7.24-7.27 (m, 1H) | 465 | 463 | Racemic form |
| F-374 | | (400 MHz, CDCl3) 0.92-1.79 (m, 17H), 2.15 (s, 3H), 2.17 (s, 3H), 2.26 (s, 3H), 2.74-2.89 (m, 2H), 2.96-3.02 (m, 1H), 3.30 (t, J = 11.02Hz, 1H), 3.70 (td, J = 10.67, 8.43Hz, 1H), 3.92 (td, J = 7.13, 2.01Hz, 1H), 4.23 (tt, J = 12.37, 5.64Hz, 2H), 4.45 (dd, J = 11.48, 8.46Hz, 1H), 6.95-6.98 (m, 2H), 778 (d, J = 8.12Hz, 1H), 9.04 (s, 1H) | 478 | 476 | |

TABLE 238

| Ex- | | | | | Informa- |
|---|---|---|---|---|---|
| am- | | | MS | | tion of |
| ple | Chemical structure | NMR | M + H | M − H | structure |
| F-375 | | (400 MHz, CDCl3) 0.92-1.79 (m, 17H), 2.15 (s, 3H), 2.17 (s, 3H), 2.26 (s, 3H), 2.74-2.89 (m, 2H), 2.96-3.02 (m, 1H), 3.30 (t, J = 11.02Hz, 1H), 3.70 (td, J = 10.67, 8.43Hz, 1H), 3.92 (td, J = 7.13, 2.01Hz, 1H), 4.23 (tt, J = 12.87, 5.64Hz, 2H), 4.46 (dd, J = 11.48, 8.46Hz, 1H), 6.95-6.98 (m, 2H), 7.78 (d, J = 8.12Hz, 1H), 9.04 (s, 1H) | 478 | 476 | |
| F-376 | | (400 MHz, CDCl3) 0.94 (s, 9H), 1.14-1.20 (m, 3H), 1.31-1.36 (m, 1H), 1.48-1.52 (m, 2H), 2.02 (s, 3H), 2.24 (s, 3H), 2.41-2.59 (m, 4H), 2.86 (dd, J = 13.30, 4.43Hz, 1H), 3.10-3.17 (m, 2H), 3.88- 3.95 (m, 1H), 5.07-5.11 (m, 2H), 5.71-5.82 (m, 1H), 6.93-6.94 (m, 2H), 7.31-7.33 (m, 2H) | 459 | 457 | Racemic form |
| F-377 | | (400 MHz, DMSO-D6) 0.86 (d, J = 6.68Hz, 6H), 1.06-1.28 (m, 4H), 1.29-1.40 (m, 2H), 1.43- 1.60 (m, 1H), 1.93-2.18 (m, 8H), 2.33 (s, 3H), 2.38-2.62 (m, 3H), 3.24-3.35 (m, 1H), 3.44-3.55 (m, 1H), 3.63-3.93 (m, 3H), 3.96- 4.18 (m, 2.5H), 4.19-4.30 (m, 0.5H), 6.95 (d, J = 8.24Hz, 1H), 7.01 (s, 1H), 7.13-7.25 (m, 1H), 9.63-9.74 (m, 1H) | 478 | 476 | (Hydro- chloride salt of F-318) |

TABLE 239

| Ex- | | | | | Informa- |
|---|---|---|---|---|---|
| am- | | | MS | | tion of |
| ple | Chemical structure | NMR | M + H | M − H | structure |
| F-378 | | (400 MHz, DMSO-D6) 0.86 (d, J = 6.68Hz, 6H), 1.05-1.36 (m, 6H), 1.41-1,60 (m, 1H), 1.90- 2.15 (m, 8H), 2.23 (s, 3H), 2.29 (s, 3H), 2.40-2.61 (m, 3H), 3.21- 3.33 (m, 1H), 3.42-3.53 (m, 1H), 3.60-3.88 (m, 3H), 3.95-4.28 (m, 3H), 6.95 (d, J = 8.04Hz, 1H), 7.02 (s, 1H), 7.11 (d, J = 7.80Hz, 2H), 7.14-7.24 (m, 1H), 7.47 (d, J = 8.00Hz, 2H), 9.56 (s, 1H) | 478 | 476 | (Tosilate salt of F-318) |
| F-379 | | (400 MHz, CDCl3) 0.89 (s, 9H), 1.26-1.32 (m, 2H), 1.78 (dd, J = 15.19, 7.54Hz, 2H), 2.05 (s, 3H), 2.09-2.15 (m, 2H), 2.26 (s, 3H), 2.54-2.68 (m, 3H), 3.02 (ddd, J = 39.60, 15.02, 7.48Hz, 2H), 3.62-3.80 (m, 3H), 4.42-4.50 (m, 1H), 4.94-5.02 (m, 1H), 6.92-6.98 (m, 2H), 7.27-7.33 (m, 1H) | 469 | 467 | |
| F-380 | | (400 MHz, CDCl3) 0.89 (s, 9H), 1.29 (dd, J = 9.51, 7.42Hz, 2H), 1.74-1.82 (m, 2H), 2.05 (s, 3H), 2.08-2.14 (m, 2H), 2.26 (s, 3H), 2.53-2.70 (m, 3H), 2.97 (dd, J = 14.96, 4.75Hz, 1H), 3.07 (dd, J = 15.07, 10.20Hz, 1H), 3.62-3.80 (m, 3H), 4.42-4.50 (m, 1H), 4.94- 5.03 (m, 1H), 6.93-6.98 (m, 2H), 7.28-7.33 (m, 2H) | 469 | 467 | |

TABLE 240

| | | | | | Informa- |
|---|---|---|---|---|---|
| | | | MS | | tion of |
| Example | Chemical structure | NMR | M + H | M − H | structure |
| F-381 | | (400 MHz, CDCl3) 0.88 (s, 9H), 1.01-1.11 (m, 1H), 1.17-1.40 (m, 4H), 1.57-1.68 (m, 1H), 1.73- 1.87 (m, 2H), 2.18 (s, 3H), 2.24 (s, 3H), 2.68 (t, J = 7.12 Hz, 2H), 2.85 (t, J = 7.36Hz, 2H), 2.94-3.05 (m, 1H), 3.13-3.25 (m, 1H), 4.00- 4.27 (m, 2H), 5.06-5.17 (m, 2H), 5.69-5.84 (m, 1H), 6.90 (d, J = 8.24Hz, 1H), 6.94 (s, 1H), 7.23- 7.32 (m, 1H), 8.41 (brs, 1H) | 423 | 421 | |
| F-382 | | (400 MHz, CDCl3) 0.83-1.00 (m, 2H), 1.12-1.33 (m, 2H), 1.20 (s, 9H), 2.03-2.16 (m, 6H), 2.25 (s, 3H), 2.35-2.53 (m, 2H), 2.62- 2.75 (m, 2H), 2.87-2.98 (m, 1H), 3.02-3.15 (m, 1H), 3.28 (t, J = 11.16Hz, 0.8H), 3.62-3.75 (m, 0.8H), 3.83-4.10 (m, 1.6H), 4.10- 4.28 (m, 3H), 4,43 (dd, J = 11.56, 8.68Hz, 0.8H), 6.91-7.01 (m, 2H), 7.57 (d, J = 8.68Hz, 0.2H), 7.72 (d, J = 8.00Hz, 0.8H), 8.06 (s, 0.2H), 8.97 (s, 0.8H) | 494 | 492 | Racemic form |
| F-383 | | (400 MHz, DMSO-D6) 0.86 (d, J = 6.72Hz, 6H), 1.02-1.27 (m, 4H), 1.27-1.37 (m, 2H), 1.41- 1.60 (m, 1H), 1.93-2.16 (m, 8H), 2.23 (s, 3H), 2.36-2.60 (m, 3H), 3.18-3.30 (m, 1H), 3.32-4.29 (m 7H), 6.95 (d, J = 8.24Hz, 1H), 7.01 (s, 1H), 7.13-7.24 (m, 1H), 9.57- 9.67 (m, 1H) | 478 | 476 | |

TABLE 241

| | | | | | Informa- |
|---|---|---|---|---|---|
| | | | MS | | tion of |
| Example | Chemical structure | NMR | M + H | M − H | structure |
| F-384 | | (400 MHz, CDCl3) 0.84-1.36 (m, 10H), 1.54-1.76 (m, 7H), 1.93- 2.03 (m, 1H), 2.04-2.15 (m, 1H), 2.06 (s, 3H), 2.25 (s, 3H), 2.74- 2.79 (m, 2H), 2.78 (brs, 1H), 2.88 (dd, J = 14.05, 4.85Hz, 1H), 3.00 (tt, J = 7.12, 3.96Hz, 1H), 3.10 (dd, J = 14.05, 10.03Hz, 1H), 3.64-3.81 (m, 2H), 3.94-4.03 (m, 1H), 6.93 (d, J = 7.94Hz, 1H), 6.94 (s, 1H), 7.39 (d, J = 7.94Hz, 1H), 7.86 (brs, 1H) | 439 | 437 | |
| F-385 | | (400 MHz. CDCl3) 0.84-1.35 (m, 10H), 1.55-1.77 (m,7H), 1.92- 2.03 (m, 1H), 2.04-2.15 (m, 1H), 2.06 (s, 3H), 2.25 (s, 3H), 2.73- 2.79 (m, 2H), 2.81 (brs, 1H), 2.88 (dd, J = 14.11, 4.85Hz, 1H), 3.00 (tt, J = 7.12, 3.96Hz, 1H), 3.10 (dd, J = 14.11, 10.14Hz, 1H), 3.64-3.80 (m, 2H), 3.93-4.03 (m, 1H), 6.93 (d, J = 7.94Hz, 1H), 6.94 (s, 1H), 7.38 (d, J = 7.94Hz, 1H), 7.87 (brs, 1H) | 439 | 437 | |
| F-386 | | (400 MHz, CDCl3) 0.87 (d, J = 6.72Hz, 6H), 0.97-1.03 (m, 1H), 1.10-1.30 (m, 5H), 1.23 (s, 3H), 1.28 (s, 3H), 1.51-1.61 (m, 1H), 1.66-1.78 (m, 1H), 1.89- 1.99 (m, 2H), 2.09 (s, 3H), 2.17 (dd, J = 14.38, 7.65Hz, 1H), 2.25 (s, 3H), 2.74 (t, J = 7.88Hz, 2H), 2.92- 3.05 (m, 3H), 3.90-3.98 (m, 1H), 6.91-6.96 (m, 2H), 7.43 (d, J = 8.58Hz, 1H), 7.87 (brs, 1H) | 441 | 439 | |

TABLE 242

| | | | | | Informa- |
|---|---|---|---|---|---|
| | | | MS | | tion of |
| Example | Chemical structure | NMR | M + H | M − H | structure |
| F-387 | | (400 MHz, CDCl3) 0.87 (d, J = 6.72Hz, 6H), 0.97-1.03 (m, 1H), 1.10-1.30 (m, 5H), 1.23 (s, 3H), 1.28 (s, 3H), 1.51-1.61 (m, 1H), 1.66-1.78 (m. 1H), 1.89- 1.99 (m, 2H), 2.09 (s, 3H), 2.17 (dd, J = 14.38, 7.65Hz, 1H), 2.25 (s, 3H), 2.74 (t, J = 7.88Hz, 2H), 2.92- 3.05 (m, 3H), 3.90-3.98 (m, 1H), 6.91-6.96 (m, 2H), 7.43 (d, J = 8.58Hz, 1H), 7.87 (brs, 1H) | 441 | 439 | |
| F-388 | | (400 MHz, CDCl3) 0.88-0.90 (m, 10H), 1.11 (s, 1H), 1.27-1.29 (m, 5H), 1.80 (s, 3H), 2.09 (s, 3H), 2.27 (s, 3H), 2.72 (s, 3H), 2.84- 2.99 (m, 3H), 3.94-3.98 (m, 1H), 5.43 (d, J = 7.72Hz, 1H), 6.97-6.99 (m, 2H), 7.46 (d, J = 8.60Hz, 1H), 7.54 (s, 1H) | 452 | 450 | |
| F-389 | | (400 MHz, CDCl3) 0.88 (s, 9H), 1.00-1.12 (m, 1H), 1.17-1.42 (m, 4H), 1.56-1.70 (m, 1H), 1.72- 1.89 (m, 2H), 2.18 (s, 3H), 2.24 (s, 3H), 2.62-2.74 (m, 2H), 2.79- 2.91 (m, 2H), 2.93-3.06 (m, 1H), 3.13-3.25 (m, 1H), 4.02-4.25 (m, 2H), 5.05-5.17 (m, 2H), 5.69- 5.84 (m, 1H), 6.91 (d, J = 7.88Hz, 1H), 6.94 (s, 1H), 7.23-7.32 (m, 1H), 8.43 (brs, 1H) | 423 | 421 | |

TABLE 243

| | | | | | Informa- |
|---|---|---|---|---|---|
| | | | MS | | tion of |
| Example | Chemical structure | NMR | M + H | M − H | structure |
| F-390 | | (400 MHz, CDCl3) 0.88 (d, J = 6.85Hz, 6H), 0.92-0.97 (m, 1H), 1.07-1.12 (m, 1H), 1.14- 1.30 (m, 8H), 1.52-1.62 (m, 1H), 1.73-1.80 (m, 2H), 1.83-1.88 (m, 1H), 2.09 (s, 3H), 2.25 (s, 3H), 2.74 (dd, J = 8.06, 8.06Hz, 2H), 2.92-3.01 (m, 2H), 3.12 (dd, J = 14.10, 9.27Hz, 1H), 3.94-4.01 (m, 2H), 6.93-6.94 (m, 2H), 7.42 (d, J = 8.46Hz, 1H), 7.96 (s, 1H) | 427 | 425 | |
| F-391 | | (400 MHz, CDCl3) 0.87 (d, J = 6.45Hz, 6H), 0.98-1.03 (m, 1H), 1.06-1.28 (m, 9H), 1.51- 1.89 (m, 3H), 2.01-2.09 (m, 1H), 2.05 (s, 3H), 2.24 (s, 3H), 2.73 (dd, J = 8.06, 7.66Hz, 2H), 2.83 (dd, J = 13.70, 4.84Hz, 1H), 3.00-3.09 (m, 2H), 3.70-3.77 (m, 1H), 3.99- 4.06 (m, 1H), 6.92-6.93 (m, 2H), 7.40 (d, J = 8.87Hz, 1H), 7.60 (s, 1H) | 427 | 425 | |
| F-392 | | (400 MHz. CDCl3) 0.86-0.95 (m, 9H), 1.17-1.34 (m, 2H), 1,76- 1.89 (m, 2H), 2.03-2.15 (m, 4H), 2.25-2.30 (m, 3H), 2.52-2.73 (m, 2H), 3.36-3.45 (m, 1H), 3.47- 3.52 (m, 2H), 3.58-3.77 (m, 2H), 3.91-4.18 (m, 2H), 4.27-4.42 (m, 2H), 6.94-7.02 (m, 2H), 7.29- 7.56 (m, 1H), 8.05 (brs, 1H) | 508 | 506 | |

TABLE 244

| | | | | | Informa- |
|---|---|---|---|---|---|
| | | | MS | | tion of |
| Example | Chemical structure | NMR | M + H | M − H | structure |
| F-393 | | (400 MHz, CDCl3) 0.85-0.95 (m, 9H), 1.19-1.33 (m, 2H), 1.75- 1.87 (m, 1H), 2.03-2.15 (m, 6H), 2.24-2.29 (m, 3H), 2.54-2.74 (m, 2H), 3.38-3.50 (m, 2H), 3.60- 3.77 (m, 2H), 3.92-4.19 (m, 2H), 4.25-4.43 (m, 2H), 4.54-5.25 (m, 1H), 6.94-7.01 (m, 2H), 7.28- 7.55 (m, 1H), 8.09 (brs, 1H) | 508 | 506 | |
| F-394 | | (400 MHz, CDCl3) 0.83-0.88 (m, 9H), 1.06-1.30 (m, 6H), 1.63- 1.74 (m, 2H), 2.00-2.07 (m, 4H), 2.14-2.19 (m, 1H), 2.21-2.24 (m, 4H), 2.25-2.31 (m, 1H), 2.35 (s, 3H), 2.66-2.73 (m, 2H), 2.88- 3.09 (m, 4H), 3.62-3.76 (m, 1H), 6.85-6.93 (m, 2H), 7.35-7.42 (m, 1H) | 452 | 450 | Racemic form |
| F-395 | | (400 MHz, CDCl3) 0.82-0.92 (m, 10H), 1.03-1.34 (m, 5H), 1.72- 1.79 (m, 2H), 1.92-2.07 (m, 2H), 2.10 (s, 3H), 2.23 (s, 3H), 2.26- 2.36 (m, 1H), 2.39 (s, 3H), 2.49- 2.65 (m, 1H), 2.67-2.74 (m, 2H), 2.88-2.97 (m, 1H), 2.97-3.06 (m, 1H), 3.12-3.20 (m, 1H), 3.22- 3.32 (m, 1H), 3.35-3.45 (m, 1H), 6.89-6.95 (m, 2H), 7.41-7.51 (m, 1H) | 452 | 450 | Racemic form |

TABLE 245

| | | | | | Informa- |
|---|---|---|---|---|---|
| | | | MS | | tion of |
| Example | Chemical structure | NMR | M + H | M − H | structure |
| F-396 | | (400 MHz, CDCl3) 0.88-0.97 (m, 3H), 1.09-1.31 (m, 8H), 1.57- 1.75 (m, 5H), 1.97-2.13 (m, 3H), 2.06 (s, 3H), 2.24 (s, 3H), 2.76- 2.87 (m, 3H), 2.93-2.98 (m, 1H), 3.06 (dd, J = 14.10, 12.89Hz, 1H), 3.23-3.31 (m, 1H), 3.27 (s, 3H), 3.43-3.49 (m, 1H), 3.89-3.97 (m, 1H), 6.91-6.93 (m, 2H), 7.43 (d, J = 8.46Hz, 1H), 7.75 (s, 1H) | 453 | 451 | Racemic form |
| F-397 | | (400 MHz, CDCl3) 0.83-1.09 (m, 2H), 0.90 (s, 9H), 1.18-1.35 (m, 4H), 1.72-1.84 (m, 2H), 2.14 (s, 3H), 2.25 (s, 3H), 2.70-2.83 (m, 2H), 2.92-3.01 (m, 1H), 2.96 (s, 3H), 3.40 (t, J = 9.80Hz, 1H), 3.81- 3.92 (m, 2H), 4.00-4.18 (m, 3H), 6.92-6.99 (m, 2H), 7.71 (d, J = 8.24Hz, 1H), 8.56 (s, 1H) | 502 | 500 | Racemic form |
| F-398 | | (400 MHz, CDCl3) 0.81-0.99 (m, 1H), 0.89 (s, 9H), 0.99-1.09 (m, 1H), 1.19-1.37 (m, 4H), 1.71- 1.84 (m, 2H), 2.13 (s, 3H), 2.25 (s, 3H), 2.69-2.84 (m, 2H), 2.90- 2.99 (m, 1H), 3.53 (t, J = 9.80Hz, 1H), 3.86-3.98 (m, 1H), 4.06 (t, J = 8.72Hz, 1H) 4.13-4.34 (m, 3H), 6.91-6.99 (m, 2H), 7.69 (d, J = 8.24Hz, 1H), 8.62 (s, 1H) | 556 | 554 | Racemic form |

TABLE 246

| | | | | | Informa- |
|---|---|---|---|---|---|
| | | | MS | | tion of |
| Example | Chemical structure | NMR | M + H | M − H | structure |
| F-399 | | (400 MHz, CDCl3) 0.82-1.08 (m, 2H), 0.89 (s, 9H), 1.15-1.36 (m, 4H), 1.71-1.83 (m, 2H), 2.12- 2.19 (m, 3H), 2.25 (s, 3H), 2.68- 2.83 (m, 2H), 2.92-3.02 (m, 1H), 3.29-3.42 (m, 1H), 3.65-3.80 (m, 3.7H), 3.82-4.18 (m, 3.6H), 4.24- 4.34 (m, 0.7H), 6.92-6.99 (m, 2H), 7.67-7.79 (m, 1H), 8.42 (s, 0.3H), 8.76 (s, 1H) | 482 | 480 | Racemic form |
| F-400 | | (400 MHz, CDCl3) 0.84-1.10 (m, 2H), 0.89 (s, 9H), 1.12-1.37 (m, 7H), 1.71-1.84 (m, 2H), 2.10 (s, 0.6H), 2.17 (s, 2.4H), 2.20-2.47 (m, 2H), 2.25 (s, 3H), 2.70-2.85 (m, 2H), 2.93-3.04 (m, 1H), 3.31 (t, J = 11.16Hz, 0.8H), 3.49-3.75 (m, 1H), 3.84-3.97 (m, 1.2H), 4.00-4.12 (m, 0.4H), 4.14-4.28 (m, 1.8H), 4.47 (dd, J = 11.56, 8.44Hz, 0.8H), 6.91-6.99 (m, 2H), 7.62 (d, J = 8.24Hz, 0.2H), 7.77 (d, J = 8.24Hz, 0.8H), 8.04 (s, 0.2H), 9.02 (s, 0.8H) | 480 | 478 | Racemic form |
| F-401 | | (400 MHz, CDCl3) 0.88 (s, 9H), 1.25-1.31 (m, 2H), 1.49-2.10 (m, 7H), 2.02 (s, 3H), 2.26 (s, 3H), 2.49-2.56 (m, 1H), 2.63-2.71 (m, 1H), 2.90-2.96 (m, 2H), 3.37- 3.45 (m, 1H), 3.57-3.68 (m, 2H), 4.25-4.33 (m, 1H), 5.07-5.15 (m, 1H), 6.92-6.96 (m, 2H), 7.22- 7.29 (m, 2H) | 483 | 481 | Racemic form |

TABLE 247

| | | | | | Informa- |
|---|---|---|---|---|---|
| Exam- | | | MS | | tion of |
| ple | Chemical structure | NMR | M + H | M − H | structure |
| F-402 | | (400 MHz, DMSO- D6) 0.88-1.76 (m, 17H), 1.97- 2.00 (m, 3H), 2.08- 2.09 (m, 3H), 2.22 (s, 3H), 2.91- 2.95 (m, 2H), 3.30- 4.22 (m, 8H), 6.93 (d, J = 8.12Hz, 1H), 7,00 (s, 1H). 7.17 (dd, J = 13.91, 8.12Hz, 1H), 9.61 (d, J = 9.74Hz, 1H) | 478 | 476 | |
| F-403 | | (400 MHz, CDCl3) 0.89 (s, 9H), 1.08-1.16 (m, 1H), 1.22-1.38 (m, 3H), 1.44-1.52 (m, 1H), 1.65- 1.74 (m, 1H), 1.79- 1.90 (m, 2H), 1.92-2.02 (m, 1H), 2.04-2.14 (m, 1H), 2.20 (s, 3H), 2.24 (s, 3H), 2.29-2.50 (m, 2H), 2.85-2.94 (m, | 466 | 464 | |
| | | 2H), 3.02-3.11 | | | |
| | | (m, 1H), 3.25- | | | |
| | | 3.35 (m, 1H), | | | |
| | | 3.48-3.57 (m, 1H), | | | |
| | | 3.66-3.76 (m, 1H), | | | |
| | | 4.35-4.48 (m, | | | |
| | | 1H), 6.43 (dd, J = | | | |
| | | 11.80, 4.52Hz, | | | |
| | | 1H), 6.90 (d, J = | | | |
| | | 7.94Hz, 1H), 6.95 | | | |
| | | (s, 1H), 7.22- | | | |
| | | 7.27 (m, 1H), 8.44 | | | |
| | | (brs, 1H) | | | |
| F-404 | | (400 MHz, CDCl3) 0.63-0.68 (m, 2H), 0.92 (d, J = 6.62Hz, 6H), 1.02- 1.09 (m, 2H), 1.86- 1.96 (m, 1H), 2.21 (s, 3H), 2.26 (s, 3H), 2.54 (d, J = 7.28Hz, 2H), 3.10-3.15 (m, | 417 | 415 | |
| | | 2H), 3.20-3.30 | | | |
| | | (m, 3H), 6.95- | | | |
| | | 6.99 (m, 2H), 7.24 | | | |
| | | (d, J = 8.16Hz, | | | |
| | | 2H), 7.61 (d, J = | | | |
| | | 8.38Hz, 2H), 7.64 | | | |
| | | (d, J = 8.82Hz, 1H), | | | |
| | | 8.17 (brs, 1H) | | | |

TABLE 248

| | | | | | Informa- |
|---|---|---|---|---|---|
| Exam- | | | MS | | tion of |
| ple | Chemical structure | NMR | M + H | M − H | structure |
| F-405 | | (400 MHz, CDCl3) 0.87-0.94 (m, 2H), 1.00 (d, J = 6.62Hz. 6H), 1.24- 1.31 (m, 2H), 2.05-2.15 (m, 1H), 2.21 (s, 3H), 2.26 (s, 3H), 2.71 (d, J = 7.06Hz, 2H), 3.12 (t, J = 6.62Hz, 2H), 3.26-3.35 (m, 3H), 6.58 (d, | 408 | 406 | |
| | | J = 0.66Hz, 1H), | | | |
| | | 6.95-7.00 (m, | | | |
| | | 2H), 7.60 (d, J = | | | |
| | | 8.82Hz, 1H), 7.95 | | | |
| | | (brs, 1H) | | | |
| F-406 | | (400 MHz, DMSO- D6) 0.53-0.63 (m, 1H), 0.70- 0.78 (m, 1H), 0.90 (d, J = 6.62Hz, 6H), 0.96-1.05 (m, 1H), 1.07-1.16 (m, 1H), 1.87- 1.96 (m, 1H), 2.00 and 2.03 (each s, total 3H), 2.09 and 2.10 (each s, total 3H), 2.24 (s, 3H), 2.56 (d, | 500 | 498 | |
| | | J = 7.28Hz, 2H), | | | |
| | | 3.44-4.29 (m, | | | |
| | | 7H), 6.96 (d, J = | | | |
| | | 7.72Hz, 1H), 7.01 | | | |
| | | (s, 1H), 7.21 and | | | |
| | | 7.24 (each d, | | | |
| | | J = 7.94 and 8.16Hz, | | | |
| | | total 1H), 7.37 | | | |
| | | (d, J = 7.50Hz, | | | |
| | | 2H), 7.71 (d, | | | |
| | | J = 7.72Hz, 2H), | | | |
| | | 9.61 (d, J = 9.70Hz, | | | |
| | | 1H) | | | |
| F-407 | | (400 MHz, DMSO- D6) 0.53-0.63 (m, 1H), 0.70- 0.78 (m, 1H), 0.90 (d, J = 6.62Hz, 6H), 0.96-1.05 (m, 1H), 1.07-1.16 (m, 1H), 1.87- 1.96 (m, 1H), 2.00 and 2.03 (each s, total 3H), 2.09 and 2.10 (each s, total 3H), 2.24 (s, 3H), 2.56 (d, | 500 | 496 | |
| | | J = 7.28Hz, 2H), | | | |
| | | 3.44-4.29 (m, | | | |
| | | 7H), 6.96 (d, J = | | | |
| | | 7.72Hz, 1H), 7.01 | | | |
| | | (s, 1H), 7.21 and | | | |
| | | 7.24 (each d, | | | |
| | | J = 7.94 and 8.16Hz, | | | |
| | | total 1H), 7.37 | | | |
| | | (d, J = 7.50Hz, | | | |
| | | 2H), 7.71 (d, | | | |
| | | J = 7.72Hz, 2H), | | | |
| | | 9.61 (d, J = 9.70Hz, | | | |
| | | 1H) | | | |

TABLE 249

| | | | | | Informa- |
|---|---|---|---|---|---|
| Exam- | | | MS | | tion of |
| ple | Chemical structure | NMR | M + H | M − H | structure |
| F-408 | | (400 MHz, DMSO- D6) 0.78-0.98 (m, 2H), 0.95 (d, J = 6.96Hz, 6H), 1.06-1.20 (m, 2H), 1.95-2.10 (m, 1H), 1.99 and 2.02 (each s, total 3H), 2.06 (s, 3H), 2.23 (s, 3H), 2.77 (d, J = 6.96Hz, 2H), 3.36-4.24 (m, 7H), 6.77 (d, J = 1.86Hz, 1H), | 491 | 489 | |
| | | 6.94 (d, J = 8.12Hz, | | | |
| | | 1H), 7.00 (s, | | | |
| | | 1H), 7.19 and 7.22 | | | |
| | | (each d, J = 8.12 | | | |
| | | and 7.88Hz, total | | | |
| | | 1H), 9.58(d, | | | |
| | | J = 6.49Hz, 1H) | | | |
| F-409 | | (400 MHz, DMSO- D6) 0.78-0.98 (m, 2H), 0.95 (d, J = 6.96Hz, 6H), 1.06-1.20 (m, 2H), 1.95-2.10 (m, 1H), 1.99 and 2.02 (each s, total 3H), 2.06 (s, 3H), 2.23 (s, 3H), 2.77 (d, J = 6.96Hz, 2H), 3.36-4.24 (m, 7H), 6.77 (d, J = 1.86Hz, 1H), | 491 | 489 | |
| | | 6.94 (d, J = 8.12Hz, | | | |
| | | 1H), 7.00 (s, | | | |
| | | 1H), 7.19 and 7.22 | | | |
| | | (each d, J = 8.12 | | | |
| | | and 7.88Hz, total | | | |
| | | 1H), 9.58 (d, | | | |
| | | J = 6.49Hz, 1H) | | | |
| F-410 | | (400 MHz, CDCl3) 0.86 (d, J = 6.62Hz, 6H), 1.05-1.12 (m, 1H), 1.24-1.37 (m, 4H), 1.47- 1.57 (m, 2H), 1.98- 2.28 (m, 4H), 2.16 (s, 3H), 2.24 (s, 3H), 2.39- 2.59 (m, 3H), 3.01 (dd, J = 15.44, 4.41Hz, 1H), 3.31- | 439 | 437 | |
| | | 3.39 (m, 1H), | | | |
| | | 3.50-3.61 (m, 1H), | | | |
| | | 3.70-3.87 (m, | | | |
| | | 2H), 3.95-4.07 | | | |
| | | (m, 1H), 4.13- | | | |
| | | 4.23 (m, 1H), 6.91 | | | |
| | | (d, J = 7.72Hz, | | | |
| | | 1H), 6.95 (s, | | | |
| | | 1H), 7.22 (d, | | | |
| | | J = 7.94Hz, 1H), | | | |
| | | 8.63 (brs, 1H) | | | |

TABLE 250

| | | | | | Informa- |
|---|---|---|---|---|---|
| Exam- | | | MS | | tion of |
| ple | Chemical structure | NMR | M + H | M − H | structure |
| F-411 | | (400 MHz, DMSO- D6) 0.50-0.80 (m, 2H), 0.94- 1.14 (m, 2H), 1.24 (s, 3H), 1.26 (s, 3H), 2.01 (d, J = 10.20Hz, 3H), 2.08 (d, J = 4.17Hz, 3H), 2.22 (s, 3H), 2.95- 3.02 (m, 1H), 3.43- 3.60 (m, 2H), 3.65-4.24 (m, 5H), 6.93-7.01 (m, | 486 | 484 | |
| | | 2H), 7.21 (dd, J = | | | |
| | | 13.33, 8.00Hz, | | | |
| | | 1H), 7.43 (d, J = | | | |
| | | 8.35Hz, 2H), 7.71 | | | |
| | | (d, J = 8.12Hz, | | | |
| | | 2H), 9.59 (d, | | | |
| | | J = 9.04Hz, 1H) | | | |
| F-412 | | (400 MHz, CDCl3) 0.92 (s, 9H), 1.32-1.39 (m, 5H), 1.58 (s, 1H), 1.89 (s, 2H), 2.27 (s, 3H), 2.29 (s, 3H), 2.99-3.02 (m, 4H), 3.13 (d, J = 8.60Hz, 1H), 3.62 (s, 1H), 3.91 (t, J = 9.59Hz, 1H), 4.04 (t, | 452 | 450 | |
| | | J = 9.46Hz, 1H), | | | |
| | | 4.34 (s, 1H), 4.47 | | | |
| | | (t, J = 9.26Hz, | | | |
| | | 1H), 6.96-6.98 (m, | | | |
| | | 2H), 7.61 (d, J = | | | |
| | | 8.16Hz, 1H), 9.62 | | | |
| | | (s, 1H) | | | |
| F-413 | | (400 MHz, CDCl3) 0.90 (s, 9H), 1.13 (s, 1H), 1.27- 1.29 (m, 5H), 1.86 (s, 2H), 2.24 (s, 3H), 2.26 (s, 3H), 2.92 (s, 5H), 3.58 (s, 1H), 3.71 (s, 1H), 4.05 (s, 1H), 4.75 (s, 1H), 4.88 (d, J = 9.92Hz, 1H), 6.92- | 452 | 450 | |
| | | 6.94 (m, 2H), 7.35 | | | |
| | | (d, J = 7.94Hz, | | | |
| | | 1H), 9.61 (s, 1H) | | | |

TABLE 251

| | | | | | Informa- |
|---|---|---|---|---|---|
| Exam- | | | MS | | tion of |
| ple | Chemical structure | NMR | M + H | M − H | structure |
| F-414 | | (400 MHz, DMSO- D6) 0.85 (d, J = 6.49Hz, 2H), 1.06-1.22 (m, 4H), 1.41-1.45 (m, 2H), 1.53 (td, J = 12.81, 6.03Hz, 1H), 1.97-2.01 (m, 3H), 2.09- 2.17 (m, 5H), 2.22 (s, 3H), 2.53- 2.61 (m, 3H), 3.27- 4.25 (m, 9H), 6.94 (d, J = 8.35Hz, | 478 | 476 | |
| | | 1H), 7.00 (s, | | | |
| | | 1H), 7.18 (dd, | | | |
| | | J = 13.33, 8.00Hz, | | | |
| | | 1H), 9.67 (d, | | | |
| | | J = 13.91Hz, 1H) | | | |
| F-415 | | (400 MHz, DMSO- D6) 0.85 (d, J = 6.49Hz, 2H), 1.06-1.22 (m, 4H), 1.41-1.45 (m, 2H), 1.53 (td, J = 12.81, 6.03Hz, 1H), 1.97-2.01 (m, 3H), 2.09- 2.17 (m, 5H), 2.22 (s, 3H), 2.53-2.61 (m, 3H), 3.27- 4.25 (m, 9H), 6.94 (d, J = 8.35Hz, | 478 | 476 | |
| | | 1H), 7.00 (s, | | | |
| | | 1H), 7.18 (dd, | | | |
| | | J = 13.33, 8.00Hz, | | | |
| | | 1H), 9.67 (d, | | | |
| | | J = 13.91Hz, 1H) | | | |
| F-416 | | (400 MHz, CDCl3) 0.75-0.91 (m, 1H), 0.91-1.07 (m, 1H), 1.17- 1.30 (m, 2H), 1.45 (s, 9H), 2.04- 2.18 (m, 6H), 2.26 (s, 3H), 2.89- 3.02 (m, 1H), 3.30 (t, J = 11, 12Hz, 0.8H), 3.58-3.65 (m, 0.2H), 3,66- 3.78 (m, 0.8H), 3.80-4.00 (m, 2H), | 523 | 521 | Racemic form |
| | | 4.02-4.52 (m, | | | |
| | | 7.2H), 6.90-7.01 | | | |
| | | (m, 2H), 7.55 (d, | | | |
| | | J = 9.04Hz, 0.2H), | | | |
| | | 7.73 (d, J = 8.08Hz, | | | |
| | | 0.8H), 7.85 (s, | | | |
| | | 0.2H), 8.76 (s, 0.8H) | | | |

TABLE 252

| | | | | | Informa- |
|---|---|---|---|---|---|
| Exam- | | | MS | | tion of |
| ple | Chemical structure | NMR | M + H | M − H | structure |
| F-417 | | (400 MHz, CDCl3) 0.74-0.92 (m, 1H), 0.92-1.14 (m, 10H), 1.19- 1.33 (m, 2H), 1.97-2.20 (m, 8H), 2.26 (s, 3H), 2.92-3.05 (m, 1H), 3.24-3.37 (m, 0.8H), 3.56-4.08 (m, 3.4H), 4.08- 4.33 (m, 3H), 4.36- 4.56 (m, 2.8H), 4.63-4.70 (m, | 521 | 519 | Racemic form |
| | | 0.4H), 4.71-4.80 | | | |
| | | (m, 0.6H), 6.91- | | | |
| | | 7.03 (m, 2H), 7.48- | | | |
| | | 7.58 (m, 0.2H), | | | |
| | | 7.66-7.78 (m, | | | |
| | | 0.8H), 7.81-7.95 | | | |
| | | (m, 0.2H), 8.63- | | | |
| | | 8.82 (m, 0.8H) | | | |
| F-418 | | (400 MHz, CDCl3) 0.75-0.88 (m, 1H), 0.92-1.07 (m, 1H), 1.17- 1.33 (m, 2H), 1.37 (d, J = 6.88Hz, 6H), 2.07 (s, 0.6H), 2.09-2.18 (m, 5.4H), 2.26 (s, 3H), 2.92-3.02 (m, 1.2H), 3.08-3.20 (m, 1H), 3.27- 3.36 (m, 0.8H), 3.59-3.79 (m, 1H), | 529 | 527 | Racemic form |
| | | 3.80-4.49 (m, 9H), | | | |
| | | 6.93-7.01 (m, | | | |
| | | 2H), 7.55 (d, J = | | | |
| | | 8.92Hz, 0.2H), | | | |
| | | 7.73 (d, J = 8.00Hz, | | | |
| | | 0.8H), 7.82 (s, | | | |
| | | 0.2H), 8.72 | | | |
| | | (m, 0.8H) | | | |
| F-419 | | (400 MHz, CDCl3) 0.76-0.91 (m, 1H), 0.92-1.07 (m, 1H), 1.12 (d, J = 6.88Hz, 6H), 1.17-1.31 (m, 2H), 2.04-2.19 (m, 6H), 2.26 (s, 3H), 2.69 (s, 3H), 2.91-3.03 (m, 1H), 3.26-3.36 (m, 0.8H), 3.57- 3.66 (m, 0.2H), 3.67-3.79 (m, | 522 | 520 | Racemic form |
| | | 0.8H), 3.82-4.00 | | | |
| | | (m, 2.2H), 4.02- | | | |
| | | 4.51 (m, 8H), 6.92- | | | |
| | | 7.01 (m, 2H), | | | |
| | | 7.55 (d, J = 8.88Hz, | | | |
| | | 0.2H), 7.72 (d, | | | |
| | | J = 8.24Hz, 0.8H), | | | |
| | | 7.94 (s, 0.2H), | | | |
| | | 8.79 (m, 0.8H) | | | |

TABLE 253

| | | | | | Informa- |
|---|---|---|---|---|---|
| Exam- | | | MS | | tion of |
| ple | Chemical structure | NMR | M + H | M − H | structure |
| F-420 | | (400 MHz, CDCl3) 0.87-0.93 (m, 2H), 1.09-1.17 (m, 2H), 1.59- 1.67 (m, 4H), 1.71-1.78 (m, 2H), 2.19 (s, 3H), 2.22- 2.31 (m, 7H), 2.37-2.46 (m, 2H), 2.85-2.92 (m, | 393 | 391 | |
| | | 1H), 3.00-3.08 | | | |
| | | (m, 2H), 3.13- | | | |
| | | 3.19 (m, 2H), 3.48- | | | |
| | | 3.58 (m, 1H), | | | |
| | | 6.93-6.99 (m, 2H), | | | |
| | | 7.59-7.64 (m, | | | |
| | | 1H), 8.24 (brs, 1H) | | | |
| F-421 | | (400 MHz, CDCl3) 0.81-0.96 (m, 8H), 1.08-1.21 (m, 2H), 1.29- 1.35 (m, 2H), 1.48-1.55 (m, 2H), 1.92-2.00 (m, 1H), 2.01-2.11 (m, 2H), 2.15 (s, 3H), 2.26 (s, 3H), 2.27-2.33 (m, 2H), 2.34-2.63 (m, | 451 | 449 | Racemic form |
| | | 4H), 2.85-2.92 | | | |
| | | (m, 1H), 3.37- | | | |
| | | 3.48 (m, 1H), | | | |
| | | 3.80-3.91 (m, 2H), | | | |
| | | 4.52-4.57 (m, 1H), | | | |
| | | 6.94-6.99 (m, | | | |
| | | 2H), 7.60-7.65 (m, | | | |
| | | 1H), 8.06 (brs, | | | |
| | | 1H) | | | |
| F-422 | | (400 MHz, DMSO- D6) 0.86 (d, J = 6.45Hz, 6H), 0.95-1.03 (m, 1H), 1.11-1.15 (m, 2H), 1.21- 1.25 (m, 1H), 1.31 (t, J = 7.05Hz, 2H), 1.49-1.56 (m, 1H), 1.86- 1.96 (m, 2H), 2.03 (s, 3H), 2.21 (s, 3H), 2.39-2.54 (m, 3H), 2.87- | 486 | 484 | Racemic form |
| | | 2.95 (m, 2H), 3.04- | | | |
| | | 3.10 (m, 1H), | | | |
| | | 3.27-3.35 (m, | | | |
| | | 2H), 3.69-3.77 (m, | | | |
| | | 1H), 4.20-4.27 | | | |
| | | (m, 1H), 6.91 (d, | | | |
| | | J = 7.66Hz, 1H), | | | |
| | | 6.97 (s, 1H), 7.08 | | | |
| | | (d, J = 8.06Hz, 1H), | | | |
| | | 7.44-7.38 (m, | | | |
| | | 2H), 7.88 (s, 1H), | | | |
| | | 8.52 (s, 1H), | | | |
| | | 9.44 (s, 1H) | | | |

TABLE 254

| | | | | | Informa- |
|---|---|---|---|---|---|
| Exam- | | | MS | | tion of |
| ple | Chemical structure | NMR | M + H | M − H | structure |
| F-423 | | (400 MHz, CDCl3) 0.86-0.93 (m, 2H), 1.04-1.17 (m, 4H), 1.49- 1.75 (m, 9H), 2.05-2.12 (m, 2H), 2.20 (s, 3H), 2.27 (s, 3H), 2,50- 2.58 (m, 3H), 2.85-2.91 (m, 1H), | 419 | 421 | |
| | | 3.04-3.10 (m, 2H), | | | |
| | | 3.13-3.19 (m, | | | |
| | | 2H), 3.57-3.64 | | | |
| | | (m, 1H), 6.94- | | | |
| | | 6.98 (m, 2H), 7.62 | | | |
| | | (d, J = 8.58Hz, | | | |
| | | 1H), 8.30 (brs, 1H) | | | |
| F-424 | | (400 MHz, CDCl3) 0.88-0.94 (m, 2H), 1.04-1.10 (m, 2H), 1.11- 1.18 (m, 2H), 1.45-1.51 (m, 4H), 1.54-1.61 (m, 2H), 1.67-1.76 (m, 3H), 2.04-2.14 (m, 2H), 2.20 (s, | 419 | 421 | |
| | | 3H), 2.27 (s, | | | |
| | | 3H), 2.36 (dd, | | | |
| | | J = 16.93, 7.65Hz, | | | |
| | | 1H), 2.50 (ddd, | | | |
| | | J = 16.12, 7.77, | | | |
| | | 2.55Hz, 2H), 2.86- | | | |
| | | 2.92 (m, 1H), 3.01- | | | |
| | | 3.07 (m, 2H), | | | |
| | | 3.13-3.18 (m, 2H), | | | |
| | | 3.38-3.47 (m, | | | |
| | | 1H), 6.94-6.99 | | | |
| | | (m, 2H), 7.60- | | | |
| | | 7.65 (m, 1H), | | | |
| | | 8.25 (brs, 1H) | | | |
| F-425 | | (400 MHz, CDCl3) 0.83-0.92 (m, 2H), 0.86 (s, 9H), 1.07-1.16 (m, 2H), 1.45 (d, J = 6.72Hz, 2H) 2.06- 2.18 (m, 2H), 2.19 (s, 3H), 2.26 (s, 3H), 2.50-2.61 (m, 2H), 2.63- | 409 | 407 | |
| | | 2.77 (m, 1H), 2.83- | | | |
| | | 2.93 (m, 1H), | | | |
| | | 2.99-3.21 (m, 4H), | | | |
| | | 3.51-3.62 (m, | | | |
| | | 1H), 6.93-7.00 | | | |
| | | (m, 2H), 7.62 (d, | | | |
| | | J = 8.80Hz, 1H), | | | |
| | | 8.31 (s, 1H) | | | |

TABLE 255

| Exam- | | | MS | | Information |
|---|---|---|---|---|---|
| ple | Chemical structure | NMR | M + H | M − H | of structure |
| F-426 | | (400 MHz, CDCl3) 0.79-0.94 (m, 2H), 0.87 (s, 9H), 1.08- 1.17 (m, 2H), 1.38 (d, J = 6.48 Hz, 2H) 2.04-2.16 (m, 2H), 2.19 (s, 3H), 2.26 (s, 3H), 2.38-2.57 (m, 3H), 2.84-2.93 | 409 | 407 | |
| | | (m, 1H), 2.97-3.19 | | | |
| | | (m, 4H), 3.37-3.49 | | | |
| | | (m, 1H), 6.91-7.00 | | | |
| | | (m, 2H), 7.62 (d, | | | |
| | | J = 8.80 Hz, 1H), | | | |
| | | 8.27 (s, 1H) | | | |
| F-427 | | (400 MHz, CDCl3) 0.88-0.92 (m, 2H), 1.12-1.17 (m, 2H), 1.37-1.48 (m, 9H), 2.13-2.23 (m, 5H), 2.26 (s, 3H), 2.35- 2.49 (m, 2H), 2.55- 2.75 (m, 3H), 2.80- | 453 | 451 | Mixture of two stereoisomers (Stereo- chemistry on Cyclobutane ring: Mixture of Cis-isomer |
| | | 2.93 (m, 1H), 3.03- | | | and |
| | | 3.07 (m, 2H), 3.14- | | | Trans-isomer) |
| | | 3.19 (m, 2H), 3.44- | | | |
| | | 3.72 (m, 1H), 6.98 | | | |
| | | (d, J = 14.11 Hz, | | | |
| | | 2H), 7.58 (t, J = 4.41 | | | |
| | | Hz, 1H), 8.28 (s, 1H) | | | |
| F-428 | | (400 MHz, CDCl3) 0.88 (d, J = 6.62 Hz, 6H), 1.08-1.15 (m, 1H), 1.21-1.38 (m, 4H), 1.29 (s, 3H), 1.36 (s, 3H), 1.51-1.62 (m, 1H), 1.74-1.84 (m, 2H), 1.88- 1.95 (m, 1H), 2.00 (dd, J = 14.45, 2.98 Hz, 1H), 2.13 (s, 3H), 2.24 (s, 3H), 2.43 (dd, J = 14.56, 10.59 Hz, 1H), 2.83 (t, J = 7.72 Hz, 2H), 3.01 (dd, J = | 441 | 439 | |
| | | 15.33, 5.62 Hz, 1H), 3.32- | | | |
| | | 3.42 (m, 1H), 3.93-4.06 | | | |
| | | (m, 1H), 4.14-4.25 (m, | | | |
| | | 1H), 6.90 (d, J = 8.16 Hz, | | | |
| | | 1H), 6.93 (s, 1H), 7.20 (d, | | | |
| | | J = 8.16 Hz, 1H), 8.53 | | | |
| | | (brs, 1H) | | | |

TABLE 256

| Exam- | | | MS | | Information |
|---|---|---|---|---|---|
| ple | Chemical structure | NMR | M + H | M − H | of structure |
| F-429 | | (400 MHz, CDCl3) 0.84 (d, J = 6.40 Hz, 6H), 1.04-1.11 (m, 2H), 1.17-1.24 (m, 2H), 1.41 (t, J = 6.62 Hz, 2H), 1.48-1.59 (m, 1H), 1.93-2.07 (m, 3H), 2.19 (s, 3H), 2.26 (s, 3H), 2.93-3.22 (m, 8H), 6.95 (s, 1H), 6.96 (d, J = 8.60 Hz, 1H), 7.59 (d, J = 8.60 | 411 | 409 | Single isomer (Stereochemistry on Cyclobutane ring: Undetermined) (Isomer of F-430) |
| | | Hz, 1H), 8.17 (brs, 1H) | | | |
| F-430 | | (400 MHz, CDCl3) 0.85 (d, J = 6.62 Hz, 6H), 1.03-1.16 (m, 4H), 1.30 (t, J = 7.17 Hz, 2H), 1.46-1.58 (m, 1H), 2.19 (s, 3H), 2.26 (s, 3H), 2.34-2.44 (m, 2H), 2.50-2.59 (m, 2H), 2.68-2.80 (m, 1H), 2.93-3.05 (m, 4H), 3.14 (t, J = 6.62 Hz, 2H), 6.93-6.99 (m, | 411 | 409 | Single isomer (Stereochemistry on Cyclobutane ring: Undetermined) (Isomer of F-429) |
| | | 2H), 7.58 (d, J = 8.82 Hz, 1H), | | | |
| | | 8.19 (brs, 1H) | | | |
| F-431 | | (400 MHz, CDCl3) 0.83-0.99 (m, 8H), 1.11-1.20 (m, 2H), 1.31-1.39 (m, 2H), 1.48-1.61 (m, 1H), 1.78-1.90 (m, 1H), 2.01-2.17 (m, 5H), 2.26 (s, 3H), 2.28-2.34 (m, 2H), 2.35- 2.46 (m, 1H), 2.46-2.56 (m, 3H), 2.85-2.92 (m, 1H), 3.38- 3.50 (m, 2H), 3.69-3.81 (m, | 451 | 449 | Racemic form |
| | | 1H), 3.97-4.05 (m, 1H), 4.46- | | | |
| | | 4.53 (m, 1H), 6.92-7.00 (m, | | | |
| | | 2H), 7.65-7.72 (m, 1H), 8.32 | | | |
| | | (brs, 1H) | | | |
| F-432 | | (400 MHz, DMSO-D6) 0.89 (s, 9H), 1.09-1.34 (m, 6H), 1.67- 1.82 (m, 2H), 1.94-2.05 (m, 3H), 2.05-2.15 (m, 3H), 2.23 (s, 3H), 2.86-3.00 (m, 2H), 3.27-4.34 (m, 7H), 6.95 (d, J = 8.24 Hz, 1H), 7.01 (s, 1H), 7.13-7.24 (m, 1H), 9.60-9.71 (m, 1H) | 466 | 464 | |

TABLE 257

| | | | | | Infor- |
|---|---|---|---|---|---|
| | | | | | mation |
| Exam- | | | MS | | of struc- |
| ple | Chemical structure | NMR | M + H | M − H | ture |
| F-433 | | (400 MHz, CDCl3) 0.85 (d, J = 8.50 Hz, 6H), 1.02 (s, 1H), 1.12-1.14 (m, 2H), 1.23 (t, J = 7.17 Hz, 3H), 1.31 (t, J = 7.06 Hz, 2H), 1.50-1.54 (m, 1H), 2.04-2.08 (m, 2H), 2.14 (s, 3H), 2.26 (s, 3H), 2.39-2.48 (m, 3H), 2.80 (s, 2H), 3.10 (s, 1H), 3.28 (dd, J = 14.11, 5.73 Hz, 1H), 3.39-3.48 (m, 2H), 4.11-4.17 (m, 2H), 4.22 (d, J = 7.72 Hz, 1H), 5.45 (dd, J = 7.94, 5.73 Hz, 1H), 6.94-6.96 (m, 2H), 7.47 (d, J = 8.60 Hz, 1H), 8.48 (s, 1H) | 497 | 495 | |
| F-434 | | (400 MHz, CDCl3) 0.86 (d, J = 6.40 Hz, 6H), 0.99 (s, 2H), 1.16 (d, J = 7.06 Hz, 2H), 1.31 (t, J = 6.95 Hz, 2H), 1.51 (dd, J = 13.45, 6.62 Hz, 1H), 2.05 (t, J = 10.26 Hz, 2H), 2.17 (s, 3H), 2.25 (s, 3H), 2.37-2.50 (m, 3H), 3.03 (d, J = 3.97 Hz, 1H), 3.26-3.33 (m, 2H), 3.43-3.47 (m, 1H), 3.76 (dd, J = 10.81, 6.62 Hz, 4H), 5.22 (dd, J = 7.28, 5.29 Hz, 1H), 6.93-6.95 (m, 2H), 7.50 (d, J = 7.72 Hz, | 455 | 453 | |
| | | 1H), 8.89 (s, 1H) | | | |
| F-435 | | (400 MHz, CDCl3) 0.83-0.90 (m, 9H), 0.97-1.02 (m, 1H), 1.13-1.21 (m, 2H), 1.22-1.30 (m, 2H), 1.69-1.75 (m, 2H), 2.01-2.05 (m, 4H), 2.06-2.15 (m, 2H), 2.17 (s, 3H), 2.23 (s, 3H), 2.61-2.76 (m, 3H), 2.94-3.03 (m, 1H), 3.20-3.51 (m, 3H), 3.94-4.04 (m, 1H), 4.79-4.89 (m, 1H), 6.87-6.95 (m, 2H), 7.33-7.38 (m, 1H), 7.63 (brs, 1H) | 480 | 478 | Racemic form |

TABLE 258

| Exam- | | | MS | | Information |
|---|---|---|---|---|---|
| ple | Chemical structure | NMR | M + H | M − H | of structure |
| F-436 | | (400 MHz, CDCl3) 0.42-0.52 (m, 1H), 0.82-0.93 (m, 12H), 0.98- 1.06 (m, 1H), 1.18-1.28 (m, 2H), 1.53-1.63 (m, 2H), 1.65-1.73 (m, 1H), 2.01 (s, 3H), 2.56 (s, 3H), 2.62-2.74 (m, 1H), 2.85-3.02 (m, 2H), 3.02-3.11 (m, 1H), 3.22- 3.32 (m, 1H), 3.90-4.00 (m, 1H), 6.89-6.94 (m, 2H). 6.94-7.00 (m, 2H), 7.15-7.22 (m, 3H), 7.31- 7.36 (m, 1H), 7.67 (brs, 1H) | 473 | 471 | Racemic form |
| F-437 | | (400 MHz, DMSO-D6) 0.82-0.90 (m, 8H), 0.93-1.02 (m, 1H), 1.05- 1.17 (m, 3H), 1.18-1.28 (m, 2H), 1.61-1.73 (m, 2H), 2.00 (s, 3H), 2.20 (s, 3H), 2.54-2.63 (m, 2H), 2.66-2.74 (m, 2H), 2.80-2.90 (m, 2H), 2.93-3.05 (m, 1H), 3.08- 3.15 (m, 1H), 3.73-3.84 (m, 1H), 6.86-6.93 (m, 1H), 6.95 (brs, 1H), 7.06-7.11 (m, 1H), 9.31 (brs, 1H) | 421 | 419 | Racemic form |
| F-438 | | (400 MHz, CDCl3) 0.87-0.89 (m, 2H), 0.87 (s, 9H), 0.95 (d, J = 5.24 Hz, 6H), 1.14-1.28 (m, 5H), 1.60-1.76 (m, 4H), 2.06 (s, 3H), 2.24 (s, 3H), 2.73 (dd, J = 8.87, 8.06 Hz, 2H), 2.82 (dd, J = 13.70, 4.03 Hz, 1H), 2.91- 2.96 (m, 1H), 3.04 (dd, J = 13.70, 10.88 Hz, 1H), 3.72-3.80 (m, 1H), 6.91-6.92 (m, 2H), 7.41 (d, J = 8.87 Hz, 1H), 7.75 (s, 1H) | 439 | 437 | Racemic form |

TABLE 259

| | | | | | Infor- |
|---|---|---|---|---|---|
| | | | | | mation |
| Exam- | | | MS | | of struc- |
| ple | Chemical structure | NMR | M + H | M − H | ture |
| F-439 | | (400 MHz, CDCl3) 0.64-0.68 (m, 1H), 0.84-0.88 (m, 1H), 0.86 (d, J = 6.85 Hz, 6H), 1.00-1.06 (m, 3H), 1.32 (t, J = 7.05 Hz, 2H), 1.50-1.57 (m, 1H), 1.95-2.04 (m, 2H), 2.06 (s, 3H), 2.24 (s, 3H), 2.32-2.55 (m, 3H), 2.94 (dd, J = 13.70, 4.43 Hz, 1H), 3.16 (dd, J = 14.10, 10.88 Hz, 1H), 3.31-3.41 (m, 1H), 3.48 (d, J = 7.25 Hz, 2H), 4.16-4.24 (m, 1H), 6.91-6.93 (m, 2H), 7.19 (d, J = 3.22 Hz, 1H), | 492 | 490 | Racemic form |
| | | 7.38 (d, J = 8.06 Hz, 1H), | | | |
| | | 7.59 (s, 1H), 7.69 (d, J = | | | |
| | | 3.22 Hz, 1H) | | | |
| F-440 | | (400 MHz, CDCl3) 0.85-0.92 (m, 11H), 0.92-0.99 (m, 1H), 1.18-1.31 (m, 4H), 1.69-1.80 (m, 2H), 2.05 (s, 3H), 2.25 (s, 3H), 2.72-2.78 (m, 2H), 2.79- 2.84 (m, 1H), 2.93-3.01 (m, 1H), 3.13-3.25 (m, 2H), 4.15- 4.25 (m, 1H), 6.91-6.96 (m, 2H), 7.29-7.34 (m, 1H), 7.51 (brs, 1H) | 422 | 420 | Racemic form |
| F-441 | | (400 MHz, CDCl3) 0.65-0.71 (m, 2H), 1.08 (q, J = 6.65 Hz, 2H), 1.38 (s, 3H), 1.39 (s, 3H), 2.22 (s, 3H), 2.27 (s, 3H), 3.13 (t, J = 6.61 Hz, 2H), 3.19-3.29 (m, 3H), 4.60-4.66 (m, 1H), 6.96-7.00 (m, 4H), 7.61-7.67 (m, 3H), 8.19 (brs, 1H) | 419 | 417 | |

TABLE 260

| | | | | | Infor- |
|---|---|---|---|---|---|
| | | | | | mation |
| Exam- | | | MS | | of struc- |
| ple | Chemical structure | NMR | M + H | M − H | ture |
| F-442 | | (400 MHz, DMSO-D6) 0.57- 0.60 (brm, 2H), 0.99 (d, J = 6.26 Hz, 2H), 2.12 (s, 3H), 2.22 (s, 3H), 2.91 (t, J = 7.07 Hz, 2H), 3.10 (t, J = 7.19 Hz, 2H), 3.44-3.47 (brm, 1H), 6.86 (d, J = 8.35 Hz, 2H), 6.93 (d, J = 7.65 Hz, 1H), 6.99 (s, 1H), 7.23 (d, J = 8.12 Hz, 1H), 7.56 (d, J = 8.58 Hz, 2H), 9.35 | 377 | 375 | |
| | | (s, 1H), 9.85 (s, 1H) | | | |
| F-443 | | (400 MHz, CDCl3) 0.64-0.74 (m, 2H), 0.91-1.07 (m, 4H), 1.10-1.38 (m, 4H), 1.51-1.62 (m, 1H), 1.62-1.82 (m, 5H), 2.12 (s, 3H), 2.26 (s, 3H), 2.92 (t, J = 6.84 Hz, 2H), 3.07 (t, J = 6.84 Hz, 2H), 4.34 (t, J = 7.61 Hz, 2H), 6.88-7.02 (m, 2H), 7.59 (d, J = 7.72 Hz, 1H), 7.77 (brs, 1H) | 395 | 393 | |
| F-444 | | (400 MHz, CDCl3) 0.82-0.93 (m, 3H), 1.06-1.17 (m, 4H), 1.40-1.54 (m, 3H), 2.02-2.11 (m, 2H), 2.19 (s, 3H), 2.24- 2.32 (m, 1H), 2.27 (s, 3H), 2.46-2.57 (m, 2H), 2.87-2.93 (m, 1H), 3.02-3.07 (m, 2H), 3.13-3.19 (m, 2H), 3.39-3.48 (m, 1H), 6.94-6.99 (m, 2H), | 409 | 407 | |
| | | 7.59-7.64 (m, 1H), 8.28 | | | |
| | | (brs, 1H) | | | |
| F-445 | | (400 MHz, CDCl3) 0.89 (s, 9H), 1.26-1.32 (m, 2H), 1.84-1.90 (m, 2H), 2.14 (s, 3H), 2.25 (s, 3H), 2.29-2.34 (m, 2H), 2.66-2.86 (m, 2H), 3.31 (d, J = 12.99 Hz, 1H), 3.64-3.73 (m, 2H), 3.74-3.84 (m, 3H), 4.70-4.78 (m, 1H), 5.32-5.40 (m, 1H), 6.91-6.97 (m, 2H), 7.19- 7.22 (m, 1H), 8.58 (brs, 1H) | 467 | 469 | |

TABLE 261

| Exam- | | | MS | | Information |
|---|---|---|---|---|---|
| ple | Chemical structure | NMR | M + H | M − H | of structure |
| F-446 | | (400 MHz, DMSO-D6) 0.87 (s, 9H), 0.91-1.18 (m, 4H), 1.19- 1.30 (m, 2H), 1.60-1.74 (m, 2H), 2.05 (s, 3H), 2.22 (s, 3H), 2.58 (d, J = 4.44 Hz, 3H), 2.66-2.75 (m, 2H), 3.01-3.11 (m, 1H), 3.24- 3.38 (m, 1H), 3.39-3.47 (m, 1H), 3.72-3.88 (m, 2H), 3.90-4.03 (m, 2H), 6.19 (q, J = 4.44 Hz, 1H), 6.93 (d, J = 8.00 Hz, 1H), 6.98 (s, 1H), 7.19 (d, J = 8.24 Hz, 1H), 9.51 (s, 1H) | 481 | 479 | Racemic form |
| F-447 | | (400 MHz, DMSO-D6) 0.87 (s, 9H), 0.90-1.04 (m, 2H), 1.05- 1.18 (m, 2H), 1.20-1.30 (m, 2H), 1.61-1.73 (m, 2H), 2.03 (s, 3H), 2.22 (s, 3H), 2.66-2.82 (m, 2H), 2.77 (s, 6H), 3.03-3.12 (m, 1H), 3.42-3.50 (m, 1H), 3.55-3.63 (m, 1H), 3.64-3.99 (m, 4H), 6.92 (d, J = 8.28 Hz, 1H), 6.98 (s, 1H), 7.19 (d, J = 8.28 Hz, 1H), 9.48 (s, 1H) | 495 | 493 | Racemic form |
| F-448 | | (400 MHz, CDCl3) 0.84-1.09 (m, 2H), 0.90 (s, 9H), 1.19-1.36 (m, 4H), 1.70-1.84 (m, 2H), 2.17 (s, 3H), 2.25 (s, 3H), 2.27 (s, 3H), 2.70-2.86 (m, 2H), 2.99-3.08 (m, 1H), 3.53 (t, J = 10.04 Hz, 1H), 3.79-3.88 (m, 1H), 3.89-4.01 (m, 1H), 4.16 (t, J = 9.80 Hz, 1H), 4.21-4.32 (m, 1H), 4.32-4.41 (m, 1H), 6.11 (s, 1H), 6.91-7.01 (m, 2H), 7.77 (d, J = 8.24 Hz, 1H), 8.78 (s, 1H) | 521 | 519 | Racemic form |

TABLE 262

| | | | | | Infor- |
|---|---|---|---|---|---|
| | | | | | mation |
| Exam- | | | MS | | of struc- |
| ple | Chemical structure | NMR | M + H | M − H | ture |
| F-449 | | (400 MHz, CDCl3) 0.87 (s, 9H), 1.15-1.51 (m, 6H), 1.72- 1.95 (m, 2H), 2.13 (s, 3H), 2.21 (s, 3H), 2.76-3.06 (m, 2H), 3.40-3.56 (m, 1H), 3.56- 3.74 (m, 1H), 3.97-4.67 (m, 5H), 6.84 (d, J = 7.80 Hz, 1H), 6.88 (s, 1H), 7.18 (d, J = 7.80 Hz, 1H), 9.81 (brs, 2H), 10.26 (brs, 1H) | 424 | 422 | Racemic form |
| F-450 | | (400 MHz, CDCl3) 0.84 (d, J = 6.62 Hz, 6H), 1.15 (d, J = 6.84 Hz, 3H), 1.26-1.30 (m, 3H), 1.51 (t, J = 6.73 Hz, 1H), 1.91 (d, J = 9.26 Hz, 1H), 2.01 (s, 3H), 2.05-2.07 (m, 1H), 2.23 (s, 3H), 2.42-2.44 (m, 3H), 2.86 (s, 2H), 3.05-3.09 (m, 2H), 3.28 (t, J = 13.12 Hz, 1H), 3.38 (d, J = 8.16 Hz, 1H), 4.25 (s, 1H), 5.67 (s, 1H), 6.89-6.91 (m, | 492 | 490 | Racemic form |
| | | 2H), 7.22-7.25 (m, 1H), 8.44 (s, | | | |
| | | 1H) | | | |
| F-451 | | (400 MHz, CDCl3) 0.74 (t, J = 7.39 Hz, 3H), 0.86 (t, J = 7.20 Hz, 3H), 0.88-0.91 (m, 2H), 1.03-1.15 (m, 3H), 1.46 (q, J = 7.50 Hz, 2H), 1.57-1.62 (m, 2H), 2.12-2.22 (m, 6H), 2.26 (s, 3H), 2.85-2.91 (m, 1H), 3.04 (t, J = 6.40 Hz, 0H), 3.15 (t, J = 6.40 Hz, 0H), 3.4-3.54 (m, 1H), 6.96- | 395 | 393 | |
| | | 7.00 (m, 2H), 7.60 (d, J = 8.82 | | | |
| | | Hz, 1H), 8.26 (brs, 1H) | | | |

TABLE 263

| | | | | | Infor- |
|---|---|---|---|---|---|
| | | | | | mation |
| Exam- | | | MS | | of struc- |
| ple | Chemical structure | NMR | M + H | M − H | ture |
| F-452 | | (400 MHz, CDCl3) 0.83-0.98 (m, 2H), 1.13-1.23 (m, 2H), 1.32-1.41 (m, 4H), 1.43-1.52 (m, 4H), 1.58-1.64 (m, 1H), 2.12-2.15 (m, 6H), 2.15-2.23 (m, 2H), 2.25 (s, 3H), 2.86- 2.95 (m, 1H), 3.25-3.35 (m, 1H), 3.46-3.60 (m, 2H), 3.63- 3.73 (m, 1H), 3.85-3.97 (m, 2H), 4.16-4.27 (m, 2H), 4.37- 4.49 (m, 1H), 6.92-6.98 (m, 2H), 7.70-7.75 (m, 1H), 9.00 (brs, 1H) | 490 | 488 | Racemic form |
| F-453 | | (400 MHz, CDCl3) 0.83-0.91 (m, 2H), 0.87 (s, 9H), 0.98 (d, J = 6.85 Hz, 3H), 1.00 (d, J = 7.25 Hz, 3H), 1.13-1.18 (m, 2H), 1.21-1.28 (m, 3H), 1.68- 1.77 (m, 1H), 2.03 (s, 3H), 2.07-2.15 (m, 1H), 2.23 (s, 3H), 2.72-2.79 (m, 3H), 2.89- 2.95 (m, 1H), 3.09 (dd, J = 12.89, 12.09 Hz, 1H), 3.52- 3.58 (m, 1H), 6.90-6.92 (m, 2H), 7.37 (d, J = 8.87 Hz, 1H), 7.49 (s, 1H) | 425 | 423 | Racemic form |
| F-454 | | (400 MHz, CDCl3) 0.82-0.90 (m, 2H), 0.85 (d, J = 6.85 Hz, 6H), 1.06-1.12 (m, 3H), 1.32 (t, J = 6.85 Hz, 2H), 1.48-1.56 (m, 1H), 1.94-2.04 (m, 1H), 2.06 (s, 3H), 2.24 (s, 3H), 2.31-2.51 (m, 3H), 2.75-2.81 (m, 1H), 2.92 (dd, J = 13.90, 4.63 Hz, 1H), 3.18 (dd, J = 11.28, 10.88 Hz, 1H), 3.24 (d, J = 7.66 Hz, 2H), 3.35- 3.44 (m, 1H), 4.16-4.24 (m, | 476 | 474 | Racemic form |
| | | 1H), 6.91-6.93 (m, 2H), 7.01 | | | |
| | | (s, 1H), 7.38 (d, J = 8.06 Hz, | | | |
| | | 1H), 7.55 (s, 1H), 7.66 (s, 1H) | | | |

TABLE 264

| | | | | | Infor- |
|---|---|---|---|---|---|
| | | | | | mation |
| Exam- | | | MS | | of struc- |
| ple | Chemical structure | NMR | M + H | M − H | ture |
| F-455 | | (400 MHz, CDCl3) 0.72- 0.80 (m, 1H), 1.01 (d, J = 6.62 Hz, 6H), 1.08-1.20 (m, 3H), 2.04-2.27 (m, 3H), 2.07 (s, 3H), 2.24 (s, 3H), 2.71 (d, J = 7.06 Hz, 2H), 2.91 (dd, J = 14.11, 4.41 Hz, 1H), 3.16 (dd, J = 14.11, 10.59 Hz, 1H), 3.20-3.26 (m, 1H), 3.44- 3.51 (m, 1H), 3.59-3.66 | 542 | 540 | Racemic form |
| | | (m, 1H), 4.05-4.14 (m, | | | |
| | | 1H), 4.46 (s, 2H), 6.53 (s, | | | |
| | | 1H), 6.91-6.96 (m, 2H), | | | |
| | | 7.23-7.34 (m, 5H), 7.44 | | | |
| | | (d, J = 8.60 Hz, 1H), 7.47 | | | |
| | | (brs, 1H) | | | |
| F-456 | | (400 MHz, CDCl3) 0.82- 0.89 (m, 7H), 0.94-1.05 (m, 3H), 1.30-1.33 (m, 2H), 1.48-1.56 (m, 1H), 1.92 (q, J = 9.63 Hz, 1H), 1.99-2.20 (m, 3H), 2.04 (s, 3H), 2.24 (s, 3H), 2.32- 2.50 (m, 3H), 2.74-2.86 (m, 2H), 3.03 (dd, J = 13.67, 10.81 Hz, 1H), 3.31-3.48 (m, 2H), 3.56- | 529 | 527 | |
| | | 3.63 (m, 1H), 3.87-3.97 | | | |
| | | (m, 1H), 4.41 (d, J = 11.69 | | | |
| | | Hz, 1H), 4.47 (d, J = 11.91 | | | |
| | | Hz, 1H), 6.90-6.95 (m, | | | |
| | | 2H), 7.25-7.35 (m, 5H), | | | |
| | | 7.42 (d, J = 8.60 Hz, 1H), | | | |
| | | 7.60 (brs, 1H) | | | |
| F-457 | | (400 MHz, CDCl3) 0.79- 0.86 (m, 1H), 0.99 (d, J = 6.62 Hz, 6H), 1.10-1.30 (m, 3H), 1.98-2.20 (m, 3H), 2.08 (s, 3H), 2.25 (s, 3H), 2.69 (d, J = 7.06 Hz, 2H), 2.88 (brs, 1H), 2.98 (dd, J = 14.56, 5.29 Hz, 1H), 3.19 (dd, J = 14.56, 9.48 Hz, 1H), 3.31-3.38 (m, 1H), 3.68-3.83 (m, | 452 | 450 | |
| | | 2H), 4.08-4.17 (m, 1H), | | | |
| | | 6.49 (s, 1H), 6.91-6.97 | | | |
| | | (m, 2H), 7.37 (d, J = 8.60 | | | |
| | | Hz, 1H), 7.90 (brs,1H) | | | |

TABLE 265

| | | | | | Infor- |
|---|---|---|---|---|---|
| | | | | | mation |
| Exam- | | | MS | | of struc- |
| ple | Chemical structure | NMR | M + H | M − H | ture |
| F-458 | | (400 MHz, CDCl3) 0.79-0.86 (m, 1H), 0.99 (d, J = 6.62 Hz, 6H), 1.10-1.30 (m, 3H), 1.98-2.20 (m, 3H), 2.08 (s, 3H), 2.25 (s, 3H), 2.69 (d, J = 7.06 Hz, 2H), 2.98 (dd, J = 14.56, 5.29 Hz, 1H), 3.09 (brs, 1H), 3.19 (dd, J = 14.56, 9.48 Hz, 1H), 3.31-3.38 (m, 1H), 3.68- 3.83 (m, 2H), 4.08-4.17 (m, 1H), 6.49 (s, 1H), 6.91-6.97 (m, 2H), 7.37 (d, J = 8.60 Hz, 1H), 7.90 (brs, 1H) | 452 | 450 | |
| F-459 | | (400 MHz, CDCl3) 0.73-0.80 (m, 1H), 0.85 (d, J = 6.62 Hz, 6H), 1.09-1.22 (m, 3H), 1.31 (t, J = 7.17 Hz, 2H), 1.48-1.57 (m, 1H), 1.89-2.13 (m, 2H), 1.99 (s, 3H), 2.24 (s, 3H), 2.32-2.52 (m, 3H), 2.73 (dd, J = 13.89, 3.97 Hz, 1H), 2.84-2.91 (m, 1H), 2.98 (dd, J = 13.56, 11.14 Hz, 1H), 3.04-3.13 (m, 1H), 3.35-3.44 (m, 1H), 3.69-3.80 (m, 1H), 3.87-3.97 (m, 2H), 4.01 (t, J = 8.71 Hz, 1H), 4.14 (t, J = 8.60 Hz, 1H), 5.09 (s, 2H), 6.89-6.97 (m, 2H), 7.15-7.40 | 584 | 582 | Racemic form |
| | | (m, 7H) | | | |
| F-460 | | (400 MHz, CDCl3) 0.65-0.69 (m, 2H), 1.05-1.11 (m, 2H), 2.22 (s, 3H), 2.27 (s, 3H), 3.12-3.13 (m, 2H), 3.19-3.30 (m, 3H), 4.59- 4.62 (m, 2H), 5.31-5.36 (m, 1H), 5.42-5.49 (m, 1H), 6.05-6.12 (m, 1H), 6.96-7.03 (m, 4H), 7.63- 7.66 (m, 3H), 8.16 (brs, 1H) | 417 | 415 | |

TABLE 266

| | | | | | Infor- |
|---|---|---|---|---|---|
| | | | | | mation |
| Exam- | | | MS | | of struc- |
| ple | Chemical structure | NMR | M + H | M − H | ture |
| F-461 | | (400 MHz, DMSO-D6) 0.87 (s, 9H), 1.06-1.28 (m, 4H), 1.32-1.40 (m, 2H), 1.92-2.18 (m, 8H), 2.23 (s, 3H), 2.44- 2.63 (m, 3H), 3.21-3.33 (m, 1H), 3.42-3.52 (m, 1H), 3.61- 3.91 (m, 3H), 3.96-4.16 (m, 2.5H), 4.18-4.26 (m, 0.5H), 6.95 (d, J = 8.12 Hz, 1H), 7.02 (s, 1H), 7.12-7.24 (m, 1H), 9.60-9.74 (m, 1H) | 492 | 490 | |
| F-462 | | (400 MHz, DMSO-D6) 0.87 (s, 9H), 1.06-1.28 (m, 4H), 1.32-1.40 (m, 2H), 1.92-2.18 (m, 8H), 2.23 (s, 3H), 2.44- 2.63 (m, 3H), 3.21-3.33 (m, 1H), 3.42-3.52 (m, 1H), 3.61-3.91 (m, 3H), 3.96-4.16 (m, 2.5H), 4.18-4.26 (m, 0.5H), 6.95 (d, J = 8.12 Hz, 1H), 7.02 (s, 1H), 7.12-7.24 (m, 1H), 9.60-9.74 (m, 1H) | 492 | 490 | |
| F-463 | | (400 MHz, CDCl3) 0.64-0.70 (m, 2H), 1.01-1.12 (m, 5H), 1.85 (td, J = 14.09, 7.27 Hz, 2H), 2.22 (s, 3H), 2.27 (s, 3H), 3.13-3.28 (m, 5H), 3.98 (t, J = 6.61 Hz, 2H), 6.95-7.00 (m, 4H), 7.61-7.66 (m, 3H), 8.23 (brs, 1H) | 419 | 417 | |
| F-464 | | (400 MHz, CDCl3) 0.81-0.94 (m, 7H), 1.11-1.21 (m, 3H), 1.27-1.35 (m, 2H), 1.45-1.56 (m, 1H), 1.92-2.14 (m, 5H), 2.26 (s, 3H), 2.30-2.55 (m, 3H), 2.81-2.87 (m, 2H), 2.92- 3.02 (m, 1H), 3.03-3.12 (m, 1H), 3.14-3.25 (m, 1H), 3.37- 3.49 (m, 1H), 4.10-4.20 (m, 1H), 6.91-6.97 (m, 2H), 7.28- 7.34 (m, 1H), 7.39 (brs, 1H) | 434 | 432 | |

TABLE 267

| Exam- | | | MS | | Information |
|---|---|---|---|---|---|
| ple | Chemical structure | NMR | M + H | M − H | of structure |
| F-465 | | (400 MHz, CDCl3) 0.81-0.94 (m, 7H), 1.11-1.21 (m, 3H), 1.27- 1.35 (m, 2H), 1.45-1.56 (m, 1H), 1.92-2.14 (m, 5H), 2.26 (s, 3H), 2.30-2.55 (m, 3H), 2.81-2.87 (m, 2H), 2.92-3.02 (m, 1H), 3.03- 3.12 (m, 1H), 3.14-3.25 (m, 1H), 3.37-3.49 (m, 1H), 4.10-4.20 (m, 1H), 6.91-6.97 (m, 2H), 7.28- 7.34 (m, 1H), 7.39 (brs, 1H) | 434 | 432 | |
| F-466 | | (400 MHz, CDCl3) 0.73-0.81 (m, 1H), 0.86 (d, J = 6.40 Hz, 6H), 1.12-1.26 (m, 3H), 1.28-1.36 (m, 2H), 1.49-1.58 (m, 1H), 1.82 and 1.89 (each s, total 3H), 1.87-2.17 (m, 2H), 2.00 (s, 3H), 2.25 (s, 3H), 2.32-2.54 (m, 3H), 2.69-2.81 (m, 1H), 2.85-3.21 (m, 3H), 3.36- 3.47 (m, 1H), 3.66-3.75 (m, 1H), 3.90-4.30 (m, 4H), 6.91-6.97 (m, 2H), 7.25-7.43 (m, 2H) | 492 | 490 | |
| F-467 | | (400 MHz, CDCl3) 0.73-0.81 (m, 1H), 0.86 (d, J = 6.40 Hz, 6H), 1.12- 1.26 (m, 3H), 1.28-1.36 (m, 2H), 1.49-1.58 (m, 1H), 1.82 and 1.89 (each s, total 3H), 1.87-2.17 (m, 2H), 2.00 (s, 3H), 2.25 (s, 3H), 2.32-2.54 (m, 3H), 2.69-2.81 (m, 1H), 2.85-3.21 (m, 3H), 3.36- 3.47 (m, 1H), 3.66-3.75 (m, 1H), 3.90-4.30 (m, 4H), 6.91-6.97 (m, 2H), 7.25-7.43 (m, 2H) | 492 | 490 | |

TABLE 268

| Exam- | | | MS | | Information |
|---|---|---|---|---|---|
| ple | Chemical structure | NMR | M + H | M − H | of structure |
| F-468 | | (400 MHz, CDCl3) 0.74-1.00 (m, 8H), 1.13-1.52 (m, 6H), 1.85- 1.98 (m, 2H), 2.03 (s, 3H), 2.22 (s, 3H), 2.30-2.52 (m, 3H), 2.99- 3.24 (m, 3H), 3.42-3.54 (m, 1H), 3.63-3.78 (m, 1H), 4.04-4.33 (m, 4H), 6.82-6.92 (m, 2H), 7.11 (d, J = 7.65 Hz, 1H), 9.37 (brs, 1H), 9.55 (brs, 1H), 9.78 (brs, 1H) | 450 | 448 | Racemic form |
| F-469 | | (400 MHz, CDCl3) 0.78-0.89 (m, 7H), 1.12-1.19 (m, 3H), 1.27- 1.33 (m, 2H), 1.47-1.57 (m, 1H), 1.89-2.10 (m, 2H), 2.00 (s, 3H), 2.24 (s, 3H), 2.29 (s, 3H), 2.33- 2.58 (m, 3H), 2.76 (dd, J = 13.78, 4.30 Hz, 1H), 2.85-3.05 (m, 5H), 3.32-3.51 (m, 3H), 3.87-3.96 (m, 1H), 6.89-6.95 (m, 2H), 7.29 (d, J = 7.72 Hz, 1H), 7.68 (brs, 1H) | 464 | 462 | Racemic form |
| F-470 | | (400 MHz, CDCl3) 0.83-0.99 (m, 8H), 1.11-1.20 (m, 2H), 1.31- 1.39 (m, 2H), 1.48-1.61 (m, 1H), 1.78-1.90 (m, 1H), 2.01-2.17 (m, 5H), 2.26 (s, 3H), 2.28-2.34 (m, 2H), 2.35-2.46 (m, 1H), 2.46- 2.56 (m, 3H), 2.85-2.92 (m, 1H), 3.38-3.50 (m, 2H), 3.69-3.81 (m, 1H), 3.97-4.05 (m, 1H), 4.46- 4.53 (m, 1H), 6.92-7.00 (m, 2H), | 451 | 449 | |
| | | 7.65-7.72 (m, 1H), 8.32 (brs, 1H) | | | |

TABLE 269

| | | | | | Infor- |
|---|---|---|---|---|---|
| | | | | | mation |
| Exam- | | | MS | | of struc- |
| ple | Chemical structure | NMR | M + H | M − H | ture |
| F-471 | | (400 MHz, CDCl3) 0.83-0.99 (m, 8H), 1.11-1.20 (m, 2H), 1.31- 1.39 (m, 2H), 1.48-1.61 (m, 1H), 1.78-1.90 (m, 1H), 2.01-2.17 (m, 5H), 2.26 (s, 3H), 2.28-2.34 (m, 2H), 2.35-2.46 (m, 1H), 2.46- 2.56 (m, 3H), 2.85-2.92 (m, 1H), 3.38-3.50 (m, 2H), 3.69-3.81 (m, 1H), 3.97-4.05 (m, 1H), 4.46- 4.53 (m, 1H), 6.92-7.00 (m, 2H), 7.65-7.72 (m, 1H), 8.32 (brs, 1H) | 451 | 449 | |
| F-472 | | (400 MHz, CDCl3) 0.72-0.78 (m, 1H), 0.78-0.92 (m, 8H), 0.95- 1.05 (m, 1H), 1.11-1.21 (m, 1H), 1.21-1.28 (m, 1H), 1.42-1.50 (m, 1H), 1.59-1.73 (m, 1H), 1.87- 1.95 (m, 1H), 1.95-1.98 (m, 1H), 2.20-2.26 (m, 6H), 2.31-2.49 (m, 2H), 2.81-2.89 (m, 1H), 2.91- 2.98 (m, 1H), 2.99-3.16 (m, 1H), 3.17-3.25 (m, 1H), 3.26-3.42 (m, 2H), 3.61-3.73 (m, 1H), 4.56- 4.67 (m, 1H), 6.86-6.94 (m, 2H), | 493 | 491 | Racemic form |
| | | 7.17-7.23 (m, 1H), 8.79-8.86 (m, | | | |
| | | 1H) | | | |
| F-473 | | (400 MHz, CDCl3) 1.11-1.69 (m, 16H), 2.11-2.66 (m, 12H), 3.22- 3.45 (m, 1H), 3.57-3.88 (m, 2H), 4.13-4.55 (m, 3H), 4.59-4.85 (m, 1H), 6.91-7.02 (m, 2H), 7.37- 7.42 (m, 1H), 9.28-9.51 (m, 1H) | 490 | 488 | |
| F-474 | | (400 MHz, CDCl3) 1.02-1.67 (m, 16H), 2.11-2.47 (m, 12H), 3.08- 3.89 (m, 3H), 4.09-4.40 (m, 3H), 4.46-5.00 (m, 1H), 6.92-7.01 (m, 2H), 7.38-7.46 (m, 1H), 9.14- 9.48 (m, 1H) | 490 | 488 | |

TABLE 270

| Exam- | | | MS | | Information |
|---|---|---|---|---|---|
| ple | Chemical structure | NMR | M + H | M − H | of structure |
| F-475 | | (400 MHz, CDCl3) 0.64- 0.68 (m, 1H), 0.86 (d, J = 6.72 Hz, 6H), 1.01-1.07 (m, 3H), 1.33 (t, J = 7.07 Hz, 2H), 1.49-1.57 (m, 1H), 1.95-2.03 (m, 2H), 2.06 (s, 3H), 2.25 (s, 3H), 2.33-2.53 (m, 4H), 2.94 (dd, J = 13.80, 4.52 Hz, 1H), 3.18 (dd, J = 13.91, 10.90 Hz, 1H), 3.32- 3.39 (m, 1H), 3.49 (d, J = 7.19 Hz, 2H), 4.21 (td, J = 8.41, 5.18 Hz, 1H), 6.92- | 492 | 490 | |
| | | 6.94 (m, 2H), 7.20 (d, J = | | | |
| | | 3.25 Hz, 1H), 7.38 (d, J = | | | |
| | | 8.12 Hz, 1H), 7.61 (s, 1H), | | | |
| | | 7.70 (d, J = 3.25 Hz, 1H) | | | |
| F-476 | | (400 MHz, CDCl3) 0.64- 0.68 (m, 1H), 0.86 (d, J = 6.72 Hz, 6H), 1.01-1.07 (m, 3H), 1.33 (t, J = 7.07 Hz, 2H), 1.49-1.57 (m, 1H), 1.95-2.03 (m, 2H), 2.06 (s, 3H), 2.25 (s, 3H), 2.33-2.53 (m, 4H), 2.94 (dd, J = 13.80, 4.52 Hz, 1H), 3.18 (dd, J = 13.91, 10.90 Hz, 1H), 3.32- 3.39 (m, 1H), 3.49 (d, J = 7.19 Hz, 2H), 4.21 (td, J = 8.41, 5.18 Hz, 1H), 6.92- | 492 | 490 | |
| | | 6.94 (m, 2H), 7.20 (d, J = | | | |
| | | 3.25 Hz, 1H), 7.38 (d, J = | | | |
| | | 8.12 Hz, 1H), 7.61 (s, 1H), | | | |
| | | 7.70 (d, J = 3.25 Hz, 1H) | | | |
| F-477 | | (400 MHz, CDCl3) 0.91 (dd, J = 9.51, 6.96 Hz, 2H), 1.08-1.27 (m, 9H), 2.19- 2.23 (m, 5H), 2.27 (s, 3H), 2.50-2.61 (m, 2H), 2.88- 2.92 (m, 1H), 3.05 (t, J = 6.49 Hz, 2H), 3.16 (t, J = 6.38 Hz, 2H), 3.44 (d, J = 6.49 Hz, 2H), 3.48-3.57 | 411 | 409 | Single isomer (stereo- chemistry on Cyclobutane ring: Undeter- mined) |
| | | (m, 2H), 6.95-6.99 (m, | | | |
| | | 2H), 7.62 (d, J = 8.81 Hz, | | | |
| | | 1H), 8.22 (brs, 1H) | | | |

TABLE 271

| Exam- | | | MS | | Information |
|---|---|---|---|---|---|
| ple | Chemical structure | NMR | M + H | M − H | of structure |
| F-478 | | (400 MHz, CDCl3) 0.87- 0.91 (m, 2H), 1.12-1.20 (m, 9H), 2.18-2.27 (m, 8H), 2.57-2.67 (m, 2H), 2.87-2.91 (m, 1H), 3.05 (t, J = 6.49 Hz, 2H), 3.17 (t, J = 6.26 Hz, 2H), 3.55- 3.71 (m, 4H), 6.94-7.00 (m, 2H), 7.62 (d, J = | 411 | 409 | Single isomer (Stereo- chemistry on Cyclobutane ring: Undeter- mined |
| | | 8.58 Hz, 1H), 8.24 | | | |
| | | (brs, 1H) | | | |
| F-479 | | (400 MHz, CDCl3) 0.85 (d, J = 6.40 Hz, 6H), 1.18-1.22 (m, 3H), 1.32 (t, J = 6.95 Hz, 2H), 1.50-1.53 (m, 1H), 1.99 (t, J = 9.15 Hz, 3H), 2.06 (s, 3H), 2.23 (s, 3H), 2.38-2.46 (m, 3H), 2.95-2.99 (m, 2H), 3.30- 3.48 (m, 3H), 3.69-3.79 (m, 2H), 3.89-3.93 (m, 1H), 4.12 (s, 1H), 6.90- 6.92 (m, 2H), 7.25-7.26 (m, 1H), 8.62 (s, 1H), 10.86 (s, 1H) | 507 | 505 | |
| F-480 | | (400 MHz, CDCl3) 0.84 (d, J = 7.06 Hz, 6H), 0.96-1.00 (m, 2H), 1.17 (d, J = 7.06 Hz, 2H), 1.24 (t, J = 6.95 Hz, 2H), 1.48 (t, J = 6.62 Hz, 1H), 1.98 (dd, J = 18.86, 9.15 Hz, 2H), 2.16 (s, 3H), 2.27 (s, 3H), 2.37-2.46 (m, 3H), 2.79 (d, J = 4.85 Hz, 3H), 2.97 (d, J = 10.59 Hz, 1H), 3.22-3.26 (m, 1H), 3.43-3.50 (m, 2H), 4.02-4.13 (m, 2H), 5.40 (t, J = 6.73 Hz, 1H), 6.95-6.98 (m, 2H), 7.20 | 482 | 480 | |
| | | (s, 1H), 7.39 (d, J = 7.94 | | | |
| | | Hz, 1H), 8.89 (s, 1H) | | | |

TABLE 272

| Exam- | | | MS | | Information |
|---|---|---|---|---|---|
| ple | Chemical structure | NMR | M + H | M − H | of structure |
| F-481 | | (400 MHz, DMSO-D6) 0.71- 0.83 (m, 6H), 0.83-0.88 (m, 1H), 1.01-1.20 (m, 6H), 1.95- 2.05 (m, 3H), 2.18-2.23 (m, 4H), 2.27-2.46 (m, 5H), 2.96- 3.08 (m, 1H), 3.13-3.20 (d, J = 5.20 Hz, 3H), 3.38-3.48 (m, 1H), 3.48-3.60 (m, 1H), 4.04-4.13 (m, 1H), 6.85-6.92 (m, 1H), 6.95 (brs, 1H), 7.10- 7.19 (m, 1H), 9.19 (brs, 1H) | 477 | 475 | Racemic form |
| F-482 | | (400 MHz, CDCl3) 0.82-1.11 (m, 2H), 1.00 (d, J = 6.62 Hz, 6H), 1.13-1.28 (m, 2H), 1.68 (d, J = 5.29 Hz, 2H), 1.80- 1.92 (m, 1H), 2.13 (s, 6H), 2.25 (s, 3H), 2.34-2.47 (m, 2H), 2.53-2.68 (m, 2H), 2.89- 2.97 (m, 1H), 3.16 (s, 3H), 3.24-3.35 (m, 1H), 3.63-4.12 (m, 3H), 4.14-4.28 (m, 2H), 4.44 (dd, J = 11.47, 8.60 Hz, 1H), 6.94 (s, 1H), 6.95 (d, J = 10.81 Hz, 1H), 7.72 (d, J = 8.16 Hz, 1H), 8.95 (brs, 1H) | 508 | 506 | Racemic form (Stereo- chemistry of Cyclobutane ring: Undeter- mined) |
| F-483 | | (400 MHz, DMSO-D6) 0.82- 1.00 (m, 8H), 1.01-1.15 (m, 2H), 1.52 (d, J = 6.62 Hz, 2H), 1.63-1.75 (m, 1H), 1.97 (s, 1.5H), 2.00 (s, 1.5H), 2.04 (s, 3H), 2.14-2.35 (m, 2H), 2.22 (s, 3H), 3.00-3.08 (m, 1H), 3.13 (s, 3H), 3.35-4.13 (m, 9H), 6.93 (d, J = 7.94 Hz, 1H), 6.99 (s, 1H), 7.18 (d, J = 7.94 Hz, 0.5 H), 7.22 (d, J = 7.94 Hz, 0.5H), 9.52 (s, 0.5H), 9.54 (s, 0.5H) | 508 | 506 | Racemic form (stereo- chemistry on Cyclobutane ring: Undeter- mined |

TABLE 273

| Exam- | | | MS | | Information |
|---|---|---|---|---|---|
| ple | Chemical structure | NMR | M + H | M − H | of structure |
| F-484 | | (400 MHz, DMSO-D6) 0.69- 0.81 (m, 1H), 0.85 (d, J = 6.62 Hz, 6H), 0.99-1.15 (m, 3H), 1.28 (t, J = 6.95 Hz, 2H), 1.46-1.58 (m, 1H), 1.76-2.11 (m, 4H), 1.98 (s, 3H), 2.21 (s, 3H), 2.30-2.49 (m, 2H), 2.68- 2.82 (m, 2H), 2.77 (s, 6H), 2.84-3.00 (m, 2H), 3.42-3.56 (m, 1H), 3.65-3.76 (m, 1H), 3.84-3.94 (m, 1H), 3.99-4.08 (m, 1H), 6.90 (d, J = 7.94 Hz, 1H), 6.96 (s, 1H), 7.08 (d, J = 7.94 Hz, 1H), 9.28 (brs, 1H) | 510 | 508 | Racemic form |
| F-485 | | (400 MHz, CDCl3) 0.86 (d, J = 24.58 Hz, 6H), 0.93-1.03 (m, 1H), 1.03-1.24 (m, 4H), 1.27-1.34 (m, 2H), 1.47-1.57 (m, 1H), 1.84-1.95 (m, 1H), 2.00-2.10 (m, 1H), 2.13 (s, 3H), 2.27 (s, 3H), 2.33-2.46 (m, 2H), 2.46-2.56 (m, 2H), 2.86-2.98 (m, 1H), 3.00-3.13 (m, 1H), 3.15-3.25 (m, 1H), 3.36-3.47 (m, 1H), 3.65 (s, 3H), 4.26-4.36 (m, 1H), 6.93- 7.01 (m, 2H), 7.51 (d, J = 8.40 Hz, 1H),7.75 (brs, 1H) | 507 | 505 | Racemic form |

TABLE 274

| Exam- | | | MS | | Information |
|---|---|---|---|---|---|
| ple | Chemical structure | NMR | M + H | M − H | of structure |
| F-486 | | (400 MHz, CDCl3) 0.85 (d, J = 6.49 Hz, 6H), 0.86-0.88 (m, 1H), 1.16-1.29 (m, 5H), 1.37 (s, 3H), 1.44 (s, 3H), 1.52 (t, J = 6.72 Hz, 1H), 1.97 (t, J = 9.97 Hz, 2H), 2.07 (s, 3H), 2.24 (s, 3H), 2.45 (d, J = 8.12 Hz, 3H), 2.85-2.88 (m, 1H), 3.08- 3.12 (m, 2H), 3.41 (t, J = 8.81 Hz, 1H), 3.64-3.66 (m, 1H), 3.72-3.74 (m, 1H), 3.99 (s, 1H), 6.90-6.92 (m, | 510 | 508 | |
| | | 2H), 7.24 (d, J = 7.88 Hz, | | | |
| | | 1H), 7.68 (t, J = 10.00 Hz, | | | |
| | | 1H), 8.53 (s, 1H) | | | |
| F-487 | | (400 MHz, CDCl3) 0.85- 0.88 (m, 7H), 1.03 (s, 1H), 1.20 (d, J = 7.42 Hz, 2H), 1.33 (t, J = 6.72 Hz, 2H), 1.54 (t, J = 6.61 Hz, 1H), 2.02-2.08 (m, 3H), 2.13 (s, 3H), 2.26 (s, 3H), 2.42- 2.47 (m, 3H), 2.86 (s, 1H), 3.00 (s, 2H), 3.42 (d, J = 1.62 Hz, 3H), 3.46 (d, J = 7.88 Hz, 1H), 3.88-3.94 (m, 4H), 6.93-6.96 (m, 2H), 7.41 (d, J = 7.88 Hz, | 496 | 494 | |
| | | 2H), 8.28 (s, 1H) | | | |
| F-488 | | (400 MHz, CDCl3) 0.78- 1.02 (m, 10H), 1.19-1.70 (m, 4H), 1.38 (d, J = 6.84 Hz, 3H), 1.96-2.48 (m, 6H), 2.07 (s, 3H), 2.23 (s, 3H), 2.66-2.72 (m, 1H), 2.88- 2.98 (m, 1H), 3.25-3.35 (m, 2H), 3.50-3.59 (m, 2H), 4.36 (d, J = 11.69 Hz, 1H), 4.41 (d, J = 11.69 Hz, 1H), 6.86-6.93 (m, 2H), 7.20- 7.52 (m, 7H) | 543 | 541 | Racemic form (Stereo- chemistry of Methyl group and Benzyloxyethyl group: Undetermined) |

TABLE 275

| Exam- | | | MS | | Information |
|---|---|---|---|---|---|
| ple | Chemical structure | NMR | M + H | M − H | of structure |
| F-489 | | (400 MHz, CDCl3) 0.75- 1.08 (m, 10H), 1.17-1.69 (m, 7H), 1.96-2.56 (m, 12H), 2.65-3.09 (m, 2H), 3.24-3.75 (m, 4H), 4.29- 4.44 (m, 2H), 6.86-7.02 (m, 2H), 7.20-7.88 (m, 7H) | 543 | 541 | Mixtures of four stereoisomers |
| F-490 | | (400 MHz, CDCl3) 0.79- 0.93 (m, 1H), 0.82 (d, J = 6.72 Hz, 6H), 0.99-1.11 (m, 1H), 1.08 (d, J = 6.96 Hz, 3H), 1.15-1.25 (m, 4H), 1.39-1.51 (m, 1H), 1.87-2.01 (m, 3H), 2.17- 2.49 (m, 4H), 2.26 (s, 3H), 2.29 (s, 3H), 2.86- 2.95 (m, 1H), 3.31-3.54 (m, 3H), 3.57-3.79 (m, 3H), 6.95-7.03 (m, 2H), 7.54 (d, J = 7.88 Hz, | 453 | 451 | Single isomer (Stereochemistry of Methyl group and Hydroxyethyl group: Undetermined) (Optical isomer of F-491) |
| | | 1H), 8.83 (brs, 1H) | | | |
| F-491 | | (400 MHz, CDCl3) 0.79- 0.93 (m, 1H), 0.82 (d, J = 6.72 Hz, 6H), 0.99-1.11 (m, 1H), 1.08 (d, J = 6.96 Hz, 3H), 1.15-1.25 (m, 4H), 1.39-1.51 (m, 1H), 1.87-2.01 (m, 3H), 2.17- 2.49 (m, 4H), 2.26 (s, 3H), 2.29 (s, 3H), 2.86- 2.95 (m, 1H), 3.17-3.26 (m, 1H), 3.38-3.49 (m, 2H), 3.57-3.79 (m, 3H), 6.95-7.03 (m, 2H), 7.54 | 453 | 451 | Single isomer (Stereochemistry of Methyl group and Hydroxyethyl group: Undetermined) (Optical isomer of F-490) |
| | | (d, J = 7.88 Hz, 1H), | | | |
| | | 8.83 (brs, 1H) | | | |
| F-492 | | (400 MHz, CDCl3) 0.78- 0.93 (m, 1H), 0.85 (d, J = 6.49 Hz, 6H), 0.97-1.15 (m, 3H), 1.19-1.35 (m, 2H), 1.39 (d, J = 6.72 Hz, 3H), 1.44-1.57 (m, 1H), 1.64-1.77 (m, 1H), 1.90- 2.19 (m, 3H), 2.04 (s, 3H), 2.23 (s, 3H), 2.27- 2.51 (m, 3H), 2.84-2.92 (m, 1H), 2.99-3.11 (m, 1H), 3.21-3.40 (m, 2H), 3.53-3.74 (m, 3H), 6.83- | 453 | 451 | Single isomer (Stereochemistry of Methyl group and Hydroxyethyl group: Undetermined) (Optical isomer of F-493) |
| | | 6.93 (m, 2H), 7.21-7.29 | | | |
| | | (m, 1H), 7.62 (brs, 1H) | | | |

TABLE 276

| Exam- | | | MS | | Information |
|---|---|---|---|---|---|
| ple | Chemical structure | NMR | M + H | M − H | of structure |
| F-493 | | (400 MHz, CDCl3) 0.79- 0.92 (m, 1H), 0.85 (d, J = 6.96 Hz, 6H), 0.98-1.15 (m, 3H), 1.21-1.33 (m, 2H), 1.38 (d, J = 6.96 Hz, 3H), 1.45-1.55 (m, 1H), 1.67-1.75 (m, 1H), 1.90- 2.19 (m, 3H), 2.02 (s, 3H), 2.23 (s, 3H), 2.28- 2.50 (m, 3H), 2.85-2.92 (m, 1H), 3.00-3.10 (m, 1H), 3.29-3.41 (m, 1H), 3.45-3.73 (m, 4H), 6.88 (d, J = 8.12 Hz, 1H), | 453 | 451 | Single isomer (Stereo- chemistry of Methyl group and Hydroxyethyl group: Undetermined) (Optical isomer of F-492) |
| | | 6.91 (s, 1H), 7.23 (d, | | | |
| | | J = 8.12 Hz, 1H), 7.72 | | | |
| | | (brs, 1H) | | | |
| F-494 | | (400 MHz, CDCl3) 0.40- 0.50 (m, 2H), 0.77-1.00 (m, 4H), 1.08-1.32 (m, 4H), 1.33-1.44 (m, 1H) 1.48-1.60 (m, 2H), 1.60- 1.81 (m, 5H), 2.16 (s, 3H), 2.27 (s, 3H), 2.66- 2.76 (m, 2H), 2.86-2.95 (m, 2H), 3.04-3.12 (m, 2H), 6.94-7.03 (m, 2H), 7.52 (s, 1H), 7.65 (d, | 395 | 393 | |
| | | J = 8.04 Hz, 1H) | | | |
| F-495 | | (400 MHz, CDCl3) 0.80- 0.91 (m, 8H), 1.10-1.31 (m, 7H), 1.47 (t, J = 6.96 Hz, 2H), 2.04-2.10 (m, 2H), 2.20 (s, 3H), 2.27 (s, 3H), 2.49-2.61 (m, 3H), 2.86-2.91 (m, 1H), 3.03-3.09 (m, 2H), 3.15- 3.20 (m, 2H), 3.58-3.65 (m, 1H), 6.95-6.99 (m, | 423 | 421 | |
| | | 2H), 7.58-7.65 (m, 1H), | | | |
| | | 8.28 (s, 1H) | | | |

TABLE 277

| Exam- | | | MS | | Information |
|---|---|---|---|---|---|
| ple | Chemical structure | NMR | M + H | M − H | of structure |
| F-496 | | (400 MHz, CDCl3) 0.81- 0.93 (m, 8H), 1.12-1.30 (m, 7H), 1.39 (t, J = 6.84 Hz, 2H), 2.04-2.12 (m, 2H), 2.20 (s, 3H), 2.27 (s, 3H) 2.36-2.53 (m, 3H), 2.87-2.92 (m, 1H), 3.00-3.09 (m, 2H), 3.11- 3.19 (m, 2H), 3.37-3.47 | 423 | 421 | |
| | | (m, 1H), 6.94-6.99 (m, | | | |
| | | 2H), 7.59-7.64 (m, 1H), | | | |
| | | 8.27 (brs, 1H) | | | |
| F-497 | | (400 MHz, CDCl3) 0.87 (s, 9H), 0.91-1.00 (m, 1H), 1.02-1.12 (m, 1H), 1.13-1.32 (m, 4H), 1.69- 1.80 (m, 2H), 2.11 (s, 3H), 2.25 (s, 3H), 2.73 (t, J = 7.94 Hz, 2H), 2.92- 3.10 (m, 3H), 3.66-3.68 (brm, 1H), 3.92-3.94 (brm, 3H), 6.95 (d, J = 8.60 Hz, 1H), 6.95 (s, 1H), 7.43 (d, J = 8.60 Hz, 1H), 7.78 (s, 1H) | 413 | 411 | Racemic form |
| F-498 | | (400 MHz, CDCl3) 0.80- 0.89 (m, 7H), 1.09-1.15 (m, 2H), 1.28-1.34 (m, 2H), 1.39 (s, 9H), 1.48- 1.58 (m, 1H), 1.89-2.01 (m, 1H), 2.03-2.11 (m, 3H), 2.25 (s, 3H), 2.29- 2.53 (m, 4H), 2.63-2.73 (m, 1H), 2.76-2.87 (m, 2H), 2.94-3.09 (m, 2H), 3.36-3.47 (m, 1H), 4.02- 4.15 (m, 1H), 6.90-6.96 (m, 2H), 7.41 (d, J = 8.40 Hz, 1H), 7.61 (brs, 1H) | 509 | 507 | Racemic form |

TABLE 278

| Exam- | | | MS | | Information |
|---|---|---|---|---|---|
| ple | Example | NMR | M + H | M − H | of structure |
| F-499 | | (400 MHz, DMSO-D6) 0.80- 0.89 (m, 6H), 0.92-1.09 (m, 4H), 1.22-1.31 (m, 2H), 1.45- 1.57 (m, 1H), 1.75-1.96 (m, 2H), 2.01 (s, 3H), 2.23 (s, 3H), 2.30-2.42 (m, 1H), 2.55- 2.73 (m, 2H), 2.74-2.87 (m, 1H), 2.95-3.05 (m, 1H), 3.43- 3.56 (m, 1H), 3.86-3.97 (m, 1H), 6.86-6.95 (m, 1H), 6.97 (brs, 1H), 7.12 (d, J = 8.40 Hz, 1H), 9.26 (brs, 1H) | 453 | 451 | Racemic form |
| F-500 | | (400 MHz, CDCl3) 0.83-0.88 (m, 6H), 0.97-1.04 (m, 1H), 1.04-1.14 (m, 2H), 1.28-1.35 (m, 2H), 1.47-1.56 (m, 1H), 1.83-1.94 (m, 1H), 2.07-2.11 (m, 4H), 2.25 (s, 3H), 2.28- 2.53 (m, 4H), 2.72-2.82 (m, 1H), 2.82-2.90 (m, 5H), 2.97 (s, 3H), 3.00-3.14 (m, 2H), 3.34-3.47 (m, 1H), 4.19-4.30 (m, 1H), 6.90-6.98 (m, 2H), 7.41-7.47 (m, 1H), 7.60- 7.66 (m, 1H) | 480 | 478 | Racemic form |
| F-501 | | (400 MHz, CDCl3) 0.81-0.90 (m, 7H), 0.95-1.16 (m, 4H), 1.21-1.35 (m, 4H), 1.74-1.98 (m, 4H), 2.01-2.12 (m, 4H), 2.25 (brs, 3H), 2.28-2.53 (m, 2H), 2.68-2.77 (m, 1H), 2.78- 2.82 (m, 2H), 2.84-2.93 (m, 1H), 2.96-3.10 (m, 2H), 3.25- 3.48 (m, 4H), 4.21-4.35 (m, 1H), 6.90-6.99 (m, 2H), 7.39- 7.48 (m, 1H), 7.58-7.65 (m, 1H) | 506 | 504 | Racemic form |

TABLE 279

| Exam- | | | MS | | Information |
|---|---|---|---|---|---|
| ple | Chemical structure | NMR | M + H | M − H | of structure |
| F-502 | | (400 MHz, CDCl3) 0.81- 0.89 (m, 6H), 0.97-1.18 (m, 4H), 1.24-1.36 (m, 2H), 1.46-1.58 (m, 1H), 1.83-1.97 (m, 2H), 1.98- 2.12 (m, 6H), 2.25 (s, 3H), 2.29-2.54 (m, 3H), 2.62-2.82 (m, 1H), 2.82- 2.97 (m, 2H), 2.97-3.08 (m, 2H), 3.28-3.69 (m, 5H), 4.20-4.33 (m, 1H), 4.40-4.52 (m, 1H), 6.88- 6.96 (m, 2H), 7.30-7.44 (m, 1H), 7.79-8.15 (m, 1H) | 522 | 520 | Mixture of two stereoisomers (Mixture of diastereomers) |
| F-503 | | (400 MHz, CDCl3) 0.80- 0.90 (m, 6H), 0.97-1.17 (m, 5H), 1.22-1.36 (m, 1H), 1.46-1.58 (m, 1H), 1.83-1.96 (m, 1H), 1.96- 2.12 (m, 6H), 2.22-2.27 (m, 3H), 2.29-2.53 (m, 4H), 2.63-2.81 (m, 1H), 2.81-2.95 (m, 2H), 2.96- 3.08 (m, 2H), 3.30-3.67 (m, 5H), 4.19-4.33 (m, 1H), 4.40-4.51 (m, 1H), 6.89-6.96 (m, 2H), 7.32- 7.44 (m, 1H), 7.75-8.09 (m, 1H) | 522 | 520 | Mixture of two stereoisomers (Mixture of diastereomers) |
| F-504 | | (400 MHz, CDCl3) 0.82- 0.90 (m, 8H), 0.93-1.04 (m, 3H), 1.29-1.36 (m, 2H), 1.48-1.59 (m, 1H), 1.93-2.02 (m, 1H), 2.03 (s, 3H), 2.23 (s, 3H), 2.26-2.47 (m, 4H), 3.05- 3.11 (m, 2H), 3.12-3.23 (m, 1H), 3.28-3.39 (m, 1H), 3.86-3.91 (m, 4H), 4.18-4.28 (m, 1H), 6.53- 6.60 (m, 2H), 6.89-6.94 (m, 2H), 7.33-7.44 (m, | 516 | 514 | Racemic form |
| | | 2H), 7.62 (brs, 1H) | | | |

TABLE 280

| Exam- | | | MS | | Information |
|---|---|---|---|---|---|
| ple | Chemical structure | NMR | M + H | M − H | of structure |
| F-505 | | (400 MHz, CDCl3) 0.54- 0.65 (m, 1H), 0.77-0.89 (m, 6H), 0.92-1.03 (m, 2H), 1.07-1.18 (m, 1H), 1.21-1.33 (m, 1H), 1.46- 1.59 (m, 1H), 1.74-1.90 (m, 1H), 1.93 (s, 3H), 2.20 (s, 3H), 2.29-2.45 (m, 4H), 2.61-2.95 (m, 3H), 2.98-3.09 (m, 1H), 3.38-3.50 (m, 1H), 3.82- 4.01 (m, 1H), 5.61-5.72 (m, 1H), 6.10-6.19 (m, | 502 | 500 | Racemic form |
| | | 1H), 6.83-6.93 (m, 1H), | | | |
| | | 6.93-6.97 (m, 1H), 7.00- | | | |
| | | 7.08 (m, 1H), 7.19-7.30 | | | |
| | | (m, 1H), 9.29 (brs, 1H), | | | |
| | | 11.72 (brs, 1H) | | | |
| F-506 | | (400 MHz, CDCl3) 0.77- 1.06 (m, 8H), 1.24-1.34 (m, 2H), 2.01-2.15 (m, 1H), 2.16 (s, 3H), 2.19 (s, 3H), 2.27 (s, 3H), 2.65 (d, J = 7.19 Hz, 2H), 3.36-3.49 (m, 2H), 3.91 (dd, J = 31.77, 18.32 Hz, 2H), 4.12-4.27 (m, 2H), 4.52 (t, J = 9.86 Hz, 1H), 6.76 (s, 1H), 6.95-7.01 (m, 2H), 7.75 (d, J = 7.88 Hz, 1H), 8.65 (brs, 1H) | 491 | 489 | |
| F-507 | | (400 MHz, CDCl3) 0.78- 0.85 (m, 1H), 0.96-1.02 (m, 7H), 1.28-1.32 (m, 2H), 2.01-2.17 (m, 1H), 2.16 (s, 3H), 2.19 (s, 3H), 2.27 (s, 3H), 2.66 (d, J = 6.96 Hz, 2H), 3.34-3.41 (m, 2H), 3.91 (tdd, J = 23.86, 11.67, 7.03 Hz, 2H), 4.10-4.27 (m, 2H), 4.53 (dd, J = 11.48, 8.46 Hz, 1H), 6.77 (s, 1H), 6.95-7.00 (m, 2H), 7.75 (d, J = 8.12 Hz, 1H), 8.65 | 491 | 489 | |
| | | (s, 1H) | | | |

TABLE 281

| Exam- | | | MS | | Information |
|---|---|---|---|---|---|
| ple | Chemical structure | NMR | M + H | M − H | of structure |
| F-508 | | (400 MHz, CDCl3) 0.90 (s, 9H), 0.99-1.39 (m, 6H), 1.32 (s, 3H), 1.35 (s, 3H), 1.66-1.83 (m, 2H), 2.16 (s, 3H), 2.25 (s, 3H), 2.75 (t, J = 7.83 Hz, 2H), 2.84 (s, 3H), 2.98 (dd, J = 14.34, 6.62 Hz, 1H), 3.13 (dd, J = 14.34, 8.38 Hz, 1H), 3.22 (tt, J = 7.12, 3.96 Hz, 1H), 3.77 (dd, J = 13.89, 1.76 Hz, 1H), 3.98 (dd, J = 13.89, 6.62 Hz, 1H), 4.02-4.10 (m, 1H), 6.94 (d, J = 8.60 Hz, 1H), 6.95 (s, 1H), 7.53 (d, | 537 | 535 | Racemic form |
| | | J = 8.60 Hz, 1H), 7.83 (s, | | | |
| | | 1H) | | | |
| F-509 | | (400 MHz, DMSO-D6) 0.74- 0.81 (m, 1H), 0.85 (d, J = 6.62 Hz, 6H), 1.01-1.13 (m, 3H), 1.28 (t, J = 7.06 Hz, 2H), 1.46- 1.58 (m, 1H), 1.78-1.96 (m, 2H), 1.98 (s, 3H), 2.21 (s, 3H), 2.30-2.48 (m, 5H), 2.66-2.76 (m, 1H), 2.88-3.02 (m, 2H), 3.42-3.55 (m, 1H), 3.60-3.72 (m, 1H), 5.00 (d, J = 4.85 Hz, 1H), 5.04 (d, J = 11.91 Hz, 1H), 5.68-5.83 (m, 1H), 6.90 (d, J = 8.16 Hz, 1H), 6.95 (s, | 435 | 433 | Racemic form |
| | | 1H), 7.08 (d, J = 8.16 Hz, | | | |
| | | 1H), 9.27 (brs, 1H) | | | |

TABLE 282

| Exam- | | | MS | | Information |
|---|---|---|---|---|---|
| ple | Chemical structure | NMR | M + H | M − H | of structure |
| F-510 | | (400 MHz, CDCl3) 0.79- 0.85 (m, 1H), 0.85 (d, J = 6.62 Hz, 6H), 1.05-1.16 (m, 3H), 1.32 (t, J = 7.06 Hz, 2H), 1.47-1.71 (m, 3H), 1.79-2.13 (m, 5H), 2.04 (s, 3H), 2.24 (s, 3H), 2.30-2.53 (m, 3H), 2.81 (dd, J = 13.67, 4.41 Hz, 1H), 2.90 (tt, J = 6.73, 4.35 Hz, 1H), 3.08 (dd, J = 13.67, 10.59 Hz, 1H), 3.35-3.47 (m, 1H), 3.59- 3.67 (m, 2H), 3.68-3.79 | 453 | 451 | |
| | | (m, 1H), 6.92 (d, J = | | | |
| | | 7.72 Hz, 1H), 6.93 (s, | | | |
| | | 1H), 7.35 (d, J = 7.72 | | | |
| | | Hz, 1H), 7.87 (brs, 1H) | | | |
| F-511 | | (400 MHz, CDCl3) 0.76- 0.86 (m, 1H), 0.85 (d, J = 6.62 Hz, 6H), 1.05-1.19 (m, 3H), 1.31 (t, J = 7.06 Hz, 2H), 1.47-1.72 (m, 3H), 1.78-2.13 (m, 5H), 2.04 (s, 3H), 2.24 (s, 3H), 2.31-2.53 (m, 3H), 2.81 (dd, J = 13.67, 4.41 Hz, 1H), 2.90 (tt, J = 6.73, 4.35 Hz, 1H), 3.08 (dd, J = 13.67, 10.59 Hz, 1H), 3.35-3.46 (m, 1H), 3.59-3.68 (m, 2H), 3.68- | 453 | 451 | |
| | | 3.80 (m, 1H), 6.92 (d, J = | | | |
| | | 7.72 Hz, 1H),6.93 (s, | | | |
| | | 1H), 7.34 (d, J = 7.72 Hz, | | | |
| | | 1H), 7.89 (brs, 1H) | | | |
| F-512 | | (400 MHz, CDCl3) 0.80- 0.90 (m, 7H), 0.91-1.01 (m, 1H), 1.07-1.18 (m, 2H), 1.30-1.37 (m, 2H), 1.48-1.61 (m, 2H), 1.91- 2.11 (m, 5H), 2.24 (s, 3H), 2.34-2.59 (m, 7H), 2.83-2.91 (m, 1H), 3.34- 3.85 (m, 6H), 6.89-6.97 (m, 2H), 7.61-7.68 (m, 1H), 8.28 (brs, 1H) | 465 | 463 | Racemic form (Stereo- chemistry of Hydroxymethyl group: Undetermined) |

TABLE 283

| | | | | | Informa- |
|---|---|---|---|---|---|
| Exam- | | | MS | | tion of |
| ple | Chemical structure | NMR | M + H | M − H | structure |
| F-513 | | (400 MHz, CDCl3) 0.84- 0.90 (m, 6H), 1.16 (s, 1H), 1.33-1.40 (m, 3H), 1.50- 1.56 (m, 2H), 2.04-2.28 (m, 5H), 2.48-2.54 (m, 4H), 3.11 (d, J = 11.91 Hz, 1H), 3.44-3.61 (m, 3H), 3.74- 3.77 (m, 2H), 4.11 (s, 1H), 6.89-6.94 (m, 1H), 7.08- 7.10 (m, 1H), 7.66 (d, J = 9.04 Hz, 1H), 8.90 (s, 1H) | 463 | 461 | |
| F-514 | | (400 MHz, DMSO-D6) 0.69- 0.77 (m, 1H), 0.85 (d, J = 6.62 Hz, 6H), 1.06-1.11 (m, 2H), 1.17-1.24 (m, 1H), 1.28 (t, J = 6.73 Hz, 2H), 1.34- 1.44 (m, 1H), 1.47-1.56 (m, 1H), 1.58-1.67 (m, 1H), 1.80-2.00 (m, 6H), 2.03-2.12 (m, 1H), 2.20 (s, 3H), 2.26- 2.69 (m, 5H), 2.83 (dd, J = 14.45, 10.03 Hz, 1H), 2.93 (s, 1H), 3.44-3.58 (m, 2H), 3.79-3.87 (m, 1H), 4.95 (d, J = 6.40 Hz, 1H), 6.89 (d, J = 7.06 Hz, 1H), 6.94 (s, | 465 | 463 | |
| | | 1H), 7.05 (d, J = 7.94 Hz, | | | |
| | | 1H), 9.23 (brs, 1H) | | | |
| F-515 | | (400 MHz, DMSO-D6) 0.69- 0.77 (m, 1H), 0.85 (d, J = 6.62 Hz, 6H), 1.06-1.12 (m, 2H), 1.17-1.23 (m, 1H), 1.28 (t, J = 6.84 Hz, 2H), 1.35- 1.44 (m, 1H), 1.47-1.56 (m, 1H), 1.57-1.66 (m, 1H), 1.78- 2.00 (m, 2H), 1.93 (s, 5H), 2.03-2.11 (m, 1H), 2.20 (s, 3H), 2.33-2.67 (m, 5H), 2.78- 2.87 (m, 1H), 2.90-2.96 (m, 1H), 3.17 (d, J = 4.41 Hz, 1H), 3.44-3.59 (m, 2H), 3.79-3.88 (m, 1H), 4.95 (d, J = 6.84 (s, 1H), 7.05 (d, J = 7.94 Hz, 1H), | 465 | 463 | |
| | | 9.23 (brs, 1H) | | | |

TABLE 284

| Exam- | | | MS | | Information |
|---|---|---|---|---|---|
| ple | Chemical structure | NMR | M + H | M − H | of structure |
| F-516 | | (400 MHz, CDCl3) 0.82- 0.95 (m, 7H), 1.01-1.14 (m, 3H), 1.32-1.40 (m, 2H), 1.47-1.61 (m, 1H), 1.99-2.19 (m, 4H), 2.25 (s, 3H), 2.30-2.36 (m, 6H), 2.36-2.45 (m, 1H), 2.45-2.57 (m, 2H), 2.83- 3.02 (m, 2H), 3.20-3.30 (m, 1H), 3.40-3.53 (m, 2H), 4.43-4.50 (m, 1H), 6.90-6.98 (m, 2H), 7.54- 7.67 (m, 1H), 8.41 (brs, | 438 | 436 | |
| | | 1H) | | | |
| F-517 | | (400 MHz, CDCl3) 0.83- 0.98 (m, 8H), 1.07-1.17 (m, 2H), 1.32-1.39 (m, 2H), 1.49-1.61 (m, 1H), 1.99-2.16 (m, 5H), 2.24 (s, 3H), 2.35-2.55 (m, 5H), 2.83-2.89 (m, 1H), 3.00-3.16 (m, 2H), 3.37- 3.57 (m, 2H), 3.73-3.86 (m, 1H), 4.92 (brs, 1H), 5.00 (brs, 1H), 6.89-6.98 (m, 2H), 7.70-7.77 (m, 1H), 8.39 (brs, 1H) | 447 | 445 | Racemic form |
| F-518 | | (400 MHz, CDCl3) 0.83- 0.87 (m, 6H), 0.90-1.07 (m, 2H), 1.09-1.18 (m, 2H), 1.30-1.36 (m, 2H), 1.47-1.57 (m, 1H), 1.93- 2.17 (m, 4H), 2.27 (s, 3H), 2.32-2.56 (m, 4H), 2.83-2.96 (m, 2H), 3.09- 3.19 (m, 1H), 3.36-3.48 (m, 1H), 3.64-3.76 (m, 2H), 3.91-4.01 (m, 1H), 6.97-7.04 (m, 1H), 7.12- 7.16 (m, 1H), 7.85 (brs, 1H), 7.95-8.02 (m, 1H) | 459 | 457 | |

TABLE 285

| | | | | | Infor - |
|---|---|---|---|---|---|
| Exam- | | | MS | | mation of |
| ple | Chemical structure | NMR | M + H | M − H | structure |
| F-519 | | (400 MHz, CDCl3) 0.81- 0.90 (m, 7H), 0.91-0.96 (m, 1H), 1.02-1.09 (m, 1H), 1.11- 1.20 (m, 2H), 1.28-1.35 (m, 2H), 1.47-1.57 (m, 1H), 1.90-2.15 (m, 4H), 2.29-2.57 (m, 3H), 2.87-2.99 (m, 2H), 3.11-3.20 (m, 1H), 3.37- 3.49 (m, 1H), 3.65-3.81 (m, 2H), 3.89-3.98 (m, 1H), 6.98-7.10 (m, 2H), 7.99-8.10 (m, 1H), 8.47 (brs, 1H) | 463 | 461 | |
| F-520 | | (400 MHz, CDCl3) 0.82-0.85 (m, 6H), 1.16 (d, J = 10.00 Hz, 1H), 1.36-1.46 (m, 6H), 2.07-2.56 (m, 7H), 3.13-3.17 (m, 1H), 3.38 (s, 1H), 3.53-3.65 (m, 4H), 3.84 (s, 3H), 4.05 (s, 1H), 7.17- 7.21 (m, 2H), 7.41 (s, 1H), 10.12 (s, 1H) | 475 | 473 | |
| F-521 | | (400 MHz, CDCl3) 0.87 (d, J = 5.84 Hz, 6H), 1.06 (d, J = 10.00 Hz, 1H), 1.26 (s, 2H), 1.33-1.55 (m, 4H), 2.04-2.11 (m, 4H), 2.45-2.54 (m, 3H), 2.94 (d, J = 11.03 Hz, 1H), 3.36 (s, 1H), 3.55-3.57 (m, 2H), 3.69-3.72 (m, 2H), 4.05 (s, 1H), 4.32-4.36 (m, 2H), 7.17-7.18 (m, 2H), 7.29 (d, J = 1.54 Hz, 1H), 8.07 (s, 1H) | 493 | 491 | |

TABLE 286

| | | | | | Infor- |
|---|---|---|---|---|---|
| | | | | | mation |
| Exam- | | | MS | | of |
| ple | Chemical structure | NMR | M + H | M − H | structure |
| F-522 | | (400 MHz, CDCl3) 0.78-0.82 (m, 1H), 0.86 (d, J = 6.72 Hz, 6H), 1.09-1.19 (m, 2H), 1.19-1.28 (m, 1H), 1.29-1.37 (m, 2H), 1.48-1.58 (m, 1H), 1.86-1.99 (m, 1H), 1.97 (s, 3H), 2.01-2.24 (m, 4H), 2.24 (s, 3H), 2.34- 2.52 (m, 4H), 2.70-2.77 (m, 1H), 2.79-2.92 (m, 2H), 2.95-3.02 (m, 1H), 3.37-3.46 (m, 1H), 3.70-3.79 (m, 2H), 4.32-4.40 (m, 1H), 6.89-6.95 (m, 2H), 7.24- 7.28 (m, 1H), 7.48 (s, 1H) | 465 | 463 | |
| F-523 | | (400 MHz, CDCl3) 0.72-0.83 (m, 1H), 0.86 (d, J = 6.72 Hz, 6H), 1.02-1.18 (m, 2H), 1.19-1.33 (m, 3H), 1.48-1.58 (m, 1H), 1.88-2.24 (m, 5H), 1.96 (s, 3H), 2.24 (s, 3H), 2.32-2.60 (m, 4H), 2.74 (dd, J = 13.68, 3.94 Hz, 1H), 2.81-2.92 (m, 2H), 2.99 (dd, J = 13.68, 11.59 Hz, 1H), 3.35-3.46 (m, 1H), 3.69- 3.81 (m, 2H), 4.31-4.40 (m, 1H), 6.88-6.95 (m, 2H), 7.22-7.29 (m, 1H), 7.57 (brs, 1H) | 465 | 463 | |
| F-524 | | (400 MHz, CDCl3) 0.86 (d, J = 8.16 Hz, 6H), 0.94-0.96 (m, 1H), 1.06- 1.08 (m, 1H), 1.15-1.18 (m, 2H), 1.32 (t, J = 6.95 Hz, 2H), 1.50-1.56 (m, 1H), 1.95-2.09 (m, 4H), 2.37-2.53 (m, 3H), 2.96-2.98 (m, 2H), 3.23-3.27 (m, 1H), 3.44-3.48 (m, 1H), 3.68-3.73 (m, 2H), 3.94 (t, J = 7.39 Hz, 1H), 7.75 (d, J = 8.82 Hz, 1H), 7.84 (s, 1H), 8.18 (d, J = 8.60 Hz, 1H), 8.42 (s, 1H) | 547 | 545 | |

TABLE 287

| | | | | | Infor- |
|---|---|---|---|---|---|
| | | | | | mation |
| Exam- | | | MS | | of |
| ple | Chemical structure | NMR | M + H | M − H | structure |
| F-525 | | (400 MHz, CDCl3) 0.83- 0.87 (m, 7H), 1.12 (d, J = 10.00 Hz, 1H), 1.27-1.34 (m, 4H), 1.51-1.53 (m, 2H), 2.02 (s, 1H), 2.18-2.21 (m, 5H), 2.46-2.50 (m, 4H), 3.12-3.15 (m, 1H), 3.41 (s, 1H), 3.57-3.59 (m, 1H), 3.77-3.79 (m, 2H), 4.10 (s, 1H), 7.07-7.10 (m, 2H), 7.33 (d, J = 8.38 Hz, 1H), 9.07 (s, 1H) | 459 | 457 | |
| F-526 | | (400 MHz, CDCl3) 0.82 (d, J = 7.06 Hz, 6H), 1.14 (s, 1H), 1.29-1.31 (m, 4H), 1.49-1.51 (m, 3H), 2.10-2.13 (m, 7H), 2.42- 2.53 (m, 4H), 3.09 (d, J = 10.00 Hz, 1H), 3.41 (s, 1H), 3.55-3.58 (m, 2H), 3.75 (s, 3H), 4.08 (s, 1H), 6.94 (d, J = 8.16 Hz, 1H), 7.05-7.07 (m, 1H), 7.19 (s, 1H), 9.75 (s, 1H) | 455 | 453 | |
| F-527 | | (400 MHz, CDCl3) 0.89 (s, 9H), 1.10-1.47 (m, 6H), 1.84-2.01 (m, 2H), 2.22 (s, 3H), 2.25 (s, 3H), 2.94-3.17 (m, 2H), 3.38-3.51 (m, 1H), 3.72-3.94 (m, 2H), 4.03-4.16 (m, 1H), 4.40-4.69 (m, 3H), 6.89-6.97 (m, 2H), 7.35 (d, J = 8.00 Hz, 1H), 9.30 (s, 1H) | 556 | 554 | Racemic form |
| F-528 | | (400 MHz, DMSO-D6) 0.75-0.93 (m, 3H), 0.87 (s, 9H), 0.99-1.11 (m, 1H), 1.13-1.43 (m, 7H), 1.62-1.80 (m, 4H), 2.05 (s, 3H), 2.21 (s, 3H), 2.81- 3.80 (m, 6H), 6.90 (d, J = 7.88 Hz, 1H), 6.97 (s, 1H), 7.11 (d, J = 7.88 Hz, 1H), 9.44 (s, 1H) | 425 | 423 | |

TABLE 288

| | | | | | Informa- |
|---|---|---|---|---|---|
| | | | | | tion |
| Exam- | | | MS | | of |
| ple | Chemical structure | NMR | M + H | M − H | structure |
| F-529 | | (400 MHz, CDCl3) 0.74- 0.82 (m, 1H), 0.85 (d, J = 6.84 Hz, 6H), 1.05-1.20 (m, 3H), 1.25-1.33 (m, 5H), 1.46-1.57 (m, 1H), 1.88-1.96 (m, 1H), 2.01 (s, 3H), 2.02-2.10 (m, 1H), 2.24 (s, 3H), 2.31- 2.53 (m, 3H), 2.93-3.11 (m, 3H), 3.36-3.48 (m, 1H), 3,77-3.84 (m, 1H), 4.07-4.21 (m, 2H), 6.91 (d, J = 7.94 | 439 | 437 | Racemic form |
| | | Hz, 1H), 6.93 (s, 1H), | | | |
| | | 7.26 (d, J = 7.94 | | | |
| | | Hz, 1H), 7.76 (s, 1H) | | | |
| F-530 | | (400 MHz, CDCl3) 0.72- 0.82 (m, 1H), 0.81-0.91 (m, 7H), 1.00-1.31 (m, 7H), 1.31-1.38 (m, 2H), 1.48-1.60 (m, 1H), 1.96- 2.17 (m, 5H), 2.25 (s, 3H), 2.33-2.44 (m, 1H), 2.44-2.54 (m, 2H), 2.69-2.90 (m, 6H), 2.91-2.96 (m, 2H), 3.36- 3.52 (m, 1H), 6.65-6.73 (m, 1H), 6.89-6.97 (m, 2H), 7.45-7.51 (m, 1H), 7.87 (brs, 1H) | 494 | 492 | |
| F-531 | | (400 MHz, CDCl3) 1.10-1.40 (m, 12H), 1.40-1.68 (m, 6H), 2.13-2.40 (m, 12H), 3.19-3.87 (m, 4H), 4.04- 4.41 (m, 3H), 4.45-4.85 (m, 2H), 6.91-7.00 (m, 2H), 7.36- 7.44 (m, 1H), 9.18-9.49 (m, 1H) | 518 | 516 | |
| F-532 | | (400 MHz, CDCl3) 1.08-1.42 (m, 12H), 1.49-1.68 (m, 6H), 2.14-2.47 (m, 13H), 3.15-3.46 (m, 1H), 3.54-3.88 (m, 3H), 4.07-4.87 (m, 4H), 6.89-7.02 (m, 2H), 7.36-7.44 (m, 1H), 9.23-9.47 (m, 1H) | 518 | 516 | |

TABLE 289

| | | | | | Infor- |
|---|---|---|---|---|---|
| | | | | | mation |
| Exam- | | | MS | | of |
| ple | Chemical structure | NMR | M + H | M − H | structure |
| F-533 | | (400 MHz, CDCl3) 0.82-0.91 (m, 7H), 0.91-1.04 (m, 1H), 1.07-1.23 (m, 2H), 1.33-1.40 (m, 2H), 1.48-1.60 (m, 1H), 2.01-2.15 (m, 4H), 2.25 (m, 4H), 2.31-2.56 (m, 4H), 2.68- 2.78 (m, 1H), 2.82-2.90 (m, 1H), 2.90-3.01 (m, 1H), 3.07- 3.20 (m, 1H), 3.38-3.47 (m, 1H), 3.68-3.80 (m, 1H), 4.04- 4.18 (m, 1H), 6.91-6.99 (m, 2H), 7.68-7.75 (m, 1H), 8.54 (brs, 1H) | 449 | 447 | Racemic form |
| F-534 | | (400 MHz, CDCl3) 0.80-0.97 (m, 8H), 1.04-1.18 (m, 2H), 1.32-1.39 (m, 2H), 1.50-1.61 (m, 1H), 1.95-2.15 (m, 7H), 2.20-2.34 (m, 12H), 2.35-2.57 (m, 2H), 2.75-2.93 (m, 2H), 3.34-3.49 (m, 1H), 3.56-3.74 (m, 2H), 6.88-6.98 (m, 2H), 7.70-7.78 (m, 1H), 8.59 (brs, 1H) | 478 | 476 | Racemic form (Stereo- chemistry of Dimethyl- amino group: Undeter- mined) |
| F-535 | | (400 MHz, CDCl3) 0.87 (d, J = 5.95 Hz, 6H), 0.96-0.98 (m, 1H), 1.22- 1.57 (m, 6H), 2.06-2.08 (m, 4H), 2.29-2.31 (m, 3H), 2.44-2.52 (m, 3H), 2.76 (q, J = 15.22 Hz, 1H), 3.33 (s, 1H), 3.45- 3.47 (m, 2H), 3.64 (s, 2H), 3.83 (s, 3H), 4.02 (s, 1H), 4.21 (d, J = 5.73 Hz, 2H), 6.62-6.64 (m, 2H), 6.98 (d, J = 7.50 Hz, 1H), 7.07 (t, J = 6.51 Hz, 1H) | 469 | 467 | |

TABLE 290

| | | | | | Infor- |
|---|---|---|---|---|---|
| | | | | | mation |
| Exam- | | | MS | | of |
| ple | Chemical structure | NMR | M + H | M − H | structure |
| F-536 | | (400 MHz, CDCl3) 0.83-0.90 (m, 7H), 0.90-1.03 (m, 1H), 1.04-1.22 (m, 7H), 1.31- 1.39 (m, 2H), 1.48-1.60 (m, 1H), 2.01-2.18 (m, 3H), 2.20 (s, 3H), 2.27 (s, 3H), 2.34-2.57 (m, 3H), 2.74-2.80 (m, 1H), 2.83- 3.01 (m, 3H), 3.38-3.50 (m, 2H), 4.47-4.53 (m, 1H), 6.93-7.00 (m, 2H), 7.58-7.66 (m, 1H), 9.33 (brs, 1H) | 452 | 450 | |
| F-537 | | (400 MHz, CDCl3) 0.81-0.94 (m, 8H), 0.98-1.11 (m, 8H), 1.12-1.19 (m, 2H), 1.31-1.39 (m, 2H), 1.50-1.60 (m, 1H), 1.98-2.15 (m, 4H), 2.19-2.28 (m, 5H), 2.33-2.44 (m, 1H), 2.44-2.56 (m, 2H),2.78-2.86 (m, 1H), 2.91-3.02 (m, 2H), 3.35-3.49 (m, 2H), 4.66- 4.71 (m, 1H), 6.89-6.96 (m, 2H), 7.54-7.61 (m, 1H), 8.21 (brs, 1H) | 466 | 464 | |
| F-538 | | (400 MHz, CDCl3) 0.87- 0.92 (m, 11H), 0.94-1.07 (m, 2H), 1.18-1.33 (m, 2H), 1.71-1.83 (m, 2H), 2.17 (s, 3H), 2.22-2.29 (m, 3H), 2.71-2.83 (m, 2H), 2.93-3.02 (m, 1H), 3.33-3.42 (m, 1H), 3.68-3.78 (m, 1H), 3.98-4.40 (m, 4H), 6.90-6.99 (m, 2H), 7.74-7.82 (m, 1H), 8.26-8.31 (m, 1H), 8.97 (brs, 1H) | 452 | 450 | Racemic form |

TABLE 291

| | | | | | Infor- |
|---|---|---|---|---|---|
| | | | | | mation |
| Exam- | | | MS | | of |
| ple | Chemical structure | NMR | M + H | M − H | structure |
| F-539 | | (400 MHz, CDCl3) 0.80- 0.93 (m, 8H), 0.97-1.16 (M, 9H), 1.30-1.40 (m, 2H), 1.97-2.14 (m, 6H), 2.16- 2.28 (m, 6H), 2.34- 2.45 (m, 1H), 2.46- 2.56 (m, 2H), 2.82- 2.91 (m, 1H), 2.97-3.33 (m, 2H), 3.36-3.51 (m, 2H), 3.53-3.77 (m, 2H), 6.89-6.98 (m, 2H), 7.67-7.76 (m, 1H), 8.56 (brs, 1H) | 506 | 504 | Racemic form (Stereo- chemistry of isopropyl- methyl- amino group: Undeter- mined) |
| F-540 | | (400 MHz, CDC13) 0.79- 1.04 (m, 2H), 0.87 (s, 9H), 1.12-1.23 (m, 2H), 1.39 (d, J = 6.48 Hz, 2H), 1.42-1.53 (m, 9H), 2.04-2.20 (m, 2H), 2.13 (s, 3H), 2.25 (s, 3H), 2.39-2.59 (m, 3H), 2.81-2.93 (m, 1H), 3.23-3.50 (m, 2H), 3.63-4.26 (m, 5H), 6.90-6.99 (m, 2H), 7.65-7.77 (m, 1H), 8.17 (s, 0.3H), 8.72 (s, 0.7H) | 550 | 548 | |
| F-541 | | (400 MHz, DMSO-D6) 0.87 (s, 9H), 1.01-1.26 (m, 4H), 1.37 (d, J = 6.48 Hz, 2H), 2.05-2.20 (m, 2H), 2.12 (s, 3H), 2.24 (s, 3H), 2.43- 2.64 (m, 3H), 2.92-3.02 (m, 1H), 3.15-3.35 (m, 3H), 3.36-3.53 (m, 3H), 3.71- 3.85 (m, 2H), 4.25 (dd, J = 15.60, 6.88 Hz, 1H), 6.95 (d, J = 8.44 Hz, 1H), 7.01 (s, 1H), 7.18 (d, J = 8.24 Hz, 1H), 8.33 (s, 1H) | 532 | 530 | |

TABLE 292

| | | | | | Infor- |
|---|---|---|---|---|---|
| | | | | | mation |
| Exam- | | | MS | | of |
| ple | Chemical structure | NMR | M + H | M − H | structure |
| F-542 | | (400 MHz, DMSO-D6) 0.87 (s, 9H), 1.00- 1.26 (m, 4H), 1.36 (d, J = 6.68 Hz, 2H), 1.97-2.17 (m, 2H), 2.12 (s, 3H), 2.24 (s, 3H), 2.38-2.62 (m, 3H), 3.19-3.32 (m, 1H), 3.68-3.95 (m, 4H), 4.04-4.20 (m, 2H), 4.27-4.37 (m, 1H), 6.97 (d, J = 8.44 Hz, 1H), 7.02 (s, 1H), 7.21 (d, J = 8.24 Hz, 1H), 9.73 (s, 1H) | 582 | 580 | |
| F-543 | | (400 MHz, CDCl3) 0.84-0.93 (m, 2H), 0.85 (d, J = 7.06 Hz, 6H), 0.99-1.05 (m, 1H), 1.08- 1.17 (m, 2H), 1.24-1.36 (m, 2H), 1.46-1.57 (m, 1H), 1.85- 2.14 (m, 6H), 2.30-2.51 (m, 3H), 2.70 (t, J = 7.50 Hz, 2H), 2.83-2.96 (m, 4H), 3.04-3.15 (m, 1H), 3.35-3.46 (m, 1H), 3.65-3.79 (m, 2H), 3.90-3.99 (m, 1H), 6.97 (d, J = 7.50 Hz, 1H), 7.07 (t, J = 7.72 Hz, 1H), 7.58 (d, J = 7.94 Hz, 1H), 7.99 (s, 1H) | 451 | 449 | |
| F-544 | | (400 MHz, CDCl3) 0.80-0.89 (m, 7H), 0.89-0.95 (m, 1H), 1.18-1.24 (m, 1H), 1.32-1.37 (m, 2H), 1.50-1.57 (m, 1H), 1.96-2.04 (m, 2H), 2.08 (s, 3H), 2.17 (s, 3H), 2.25-2.29 (m, 3H), 2.33-2.62 (m, 6H), 2.84-2.92 (m, 1H), 3.35-3.51 (m, 2H), 3.74-3.86 (m, 1H), 4.16-4.26 (m, 1H), 4.87 (brs, 1H), 6.84-7.03 (m, 4H), 7.41- 7.48 (m, 1H), 8.53 (brs, 1H) | 468 | 466 | |

TABLE 293

| | | | | | Infor- |
|---|---|---|---|---|---|
| Exam- | | | MS | | mation of |
| ple | Chemical structure | NMR | M + H | M − H | structure |
| F-545 | | (400 MHz, CDCl3) 0.77- 0.92 (m, 8H), 0.91-1.01 (m, 1H), 1.08-1.20 (m, 2H), 1.21- 1.31 (m, 2H), 1.42-1.53 (m, 1H), 1.88-2.04 (m, 3H), 2.10 (s, 3H), 2.13-2.22 (m, 1H), 2.26 (s, 3H), 2.30-2.56 (m, 7H), 2.70-2.82 (m, 1H), 2.84- 2.95 (m, 1H), 3.33-3.47 (m, 1H), 3.90-4.16 (m, 2H), 6.91- 7.00 (brm, 1H), 7.42-7.51 (m, 1H), 8.47 (brs, 1H) | 519 | 517 | Racemic form (Stereo- chemistry of Hydroxyl group and Trifluoro- methyl group: Undeter- mined) |
| F-546 | | (400 MHz, CDCl3) 0.85 (t, J = 5.73 Hz, 6H), 1.13-1.54 (m, 8H), 2.17- 2.42 (m, 10H), 3.18 (s, 1H), 3.40 (s, 1H), 3.67-3.74 (m, 3H), 4.11 (s, 1H), 7.29-7.42 (m, 2H), 7.63 (d, J = 7.94 Hz, 1H), 9.21 (s, 1H) | 493 | 491 | |
| F-547 | | (400 MHz CDCl3) 0.99- 1.01 (m, 7H), 1.22 (s, 1H), 1.41 (s, 2H), 2.08-2.11 (m, 1H), 2.19 (s, 3H), 2.25 (s, 3H), 2.74 (d, J = 7.06 Hz, 2H), 3.55 (s, 1H), 3.87-3.93 (m, 2H), 4.25 (s, 1H), 4.44-4.49 (m, 2H), 4.59 (s, 1H), 6.89 (s, 1H), 6.93- 6.95 (m, 2H), 7.41 (d, J = 7.72 Hz, 1H), 9.06 (s, 1H) | 581 | 579 | |
| F-548 | | (400 MHz, CDCl3) 0.87 (d, J = 35.73 Hz, 6H), 1.22-1.47 (m, 7H), 2.05-2.22 (m, 9H), 2.42-2.56 (m, 5H), 3.05 (d, J = 10.37 Hz, 1H), 3.42 (s, 1H), 3.59-3.63 (m, 1H), 3.75 (s, 2H), 4.16 (s, 1H), 6.62-6.65 (m, 1H), 6.75 (s, 1H), 8.94 (s, 1H) | 457 | 455 | |

TABLE 294

| | | | | | Infor- |
|---|---|---|---|---|---|
| | | | | | mation |
| Exam- | | | MS | | of |
| ple | Chemical structure | NMR | M + H | M − H | structure |
| F-549 | | (400 MHz, DMSO-D6) 0.87 (s, 9H), 0.97-1.24 (m, 4H), 1.36 (d, J = 6.48 Hz, 2H), 1.97-2.17 (m, 2H), 2.13 (s, 3H), 2.24 (s, 3H), 2.42-2.64 (m, 3H), 2.85-3.01 (m, 3H), 3.11-3.26 (m, 1H), 3.33-3.59 (m, 1H), 3.62-4.50 (m, 6H), 6.96 (d, J = 8.24 Hz, 1H), 7.02 (s, 1H), 7.20-7.27 (m, 1H), 9.83 (s, 0.4H), 9.91 (s, 0.6H), 10.95 (brs, 1H) | 464 | 462 | |
| F-550 | | (400 MHz, DMSO-D6) 0.87 (s, 9H), 1.00-1.26 (m, 4H), 1.36 (d, J = 6.68 Hz, 2H), 1.97-2.15 (m, 2H), 2.11 (s, 3H), 2.23 (s, 3H), 2.43-2.62 (m, 3H), 3.18- 3.28 (m, 1H), 3.66-3.81 (m, 3H), 3.81-3.93 (m, 1H), 4.03-4.14 (m, 2H), 4.20-4.32 (m, 1H), 6.96 (d, J = 8.44 Hz, 1H), 7.02 (s, 1H), 7.10-7.37 (m, 1H), 7.20 (d, J = 8.04 Hz, 1H), 9.69 (s, 1H) | 564 | 562 | |
| F-551 | | (400 MHz, DMSO-D6) 0.87 (s, 9H), 1.03-1.26 (m, 4H), 1.36 (d, J = 6.44 Hz, 2H), 1.98-2.18 (m, 5H), 2.24 (s, 3H), 2.44-2.63 (m, 3H), 3.20-3.32 (m, 1H), 3.60-3.94 (m, 4H), 3.99-4.32 (m, 3H), 6.44-6.81 (m, 1H), 6.96 (d, J = 8.24 Hz, 1H), 7.02 (s, 1H), 7.16-7.25 (m, 1H), 9.66-9.77 (m, 1H) | 528 | 526 | |

TABLE 295

| | | | | | Infor- |
|---|---|---|---|---|---|
| | | | | | mation |
| Exam- | | | MS | | of |
| ple | Chemical structure | NMR | M + H | M − H | structure |
| F-552 | | (400 MHz, DMSO-D6) 0.87 (s, 9H), 1.04-1.33 (m, 10H), 1.36 (d, J = 6.68 Hz, 2H), 2.00-2.16 (m, 2H), 2.11 (s, 3H), 2.23 (s, 3H), 2.43-2.62 (m, 3H), 3.19- 3.29 (m, 1H), 3.41-3.52 (m, 1H), 3.52-3.61 (m, 2H), 3.69-3.89 (m, 2H), 3.91-4.05 (m, 2H), 4.22 (q, J = 8.04 Hz, 1H), 6.95 (d, J = 8.24 Hz, 1H), 7.01 (s, 1H), 7.19 (d, J = 8.24 Hz, 1H), 9.67 (s, 1H) | 556 | 554 | |
| F-553 | | (400 MHz, DMSO-D6) 0.87 (s, 9H), 0.96-1.27 (m, 10H), 1.36 (d, J = 6.48 Hz, 2H), 1.98- 2.19 (m, 5H), 2.23 (s, 3H), 2.40-2.80 (m, 4H), 3.21-3.32 (m, 1H), 3.44-3.55 (m, 1H), 3.64-3.91 (m, 3H), 3.94-4.05 (m, 1H), 4.07-4.27 (m, 2H), 6.95 (d, J = 8.24 Hz, 1H), 7.01 (s, 1H), 7.13-7.26 ( m, 1H), 9.59- 9.73 (m, 1H) | 520 | 518 | |
| F-554 | | (400 MHz, CDCl3) 0.83-0.96 (m, 8H), 1.06-1.19 (m, 2H), 1.33-1.40 (m, 2H), 1.50-1.62 (m, 1H), 1.77-1.86 (m, 1H), 2.02-2.13 (m, 2H), 2.15 (s, 3H), 2.16-2.23 (m, 1H), 2.25 (s, 3H), 2.35-2.56 (m, 4H), 2.65-2.74 (m, 1H), 2.82-2.91 (m, 1H), 3.30 (s, 3H), 3.32- 3.47 (m, 2H), 3.91-4.09 (m, 2H), 6.90-6.99 (m, 2H), 7.76- 7.81 (m, 1H), 8.61 (brs, 1H) | 405 | 403 | Single isomer (Stereo- chemistry of Methoxy group: Undeter- mined) (Optical isomer of F-555) |

TABLE 296

| | | | | | Infor- |
|---|---|---|---|---|---|
| | | | | | mation |
| Exam- | | | MS | | of |
| ple | Chemical structure | NMR | M + H | M − H | structure |
| F-555 | | (400 MHz, CDCl3) 0.84-0.99 (m, 8H), 1.02-1.20 (m, 2H), 1.32-1.40 (m, 2H), 1.48-1.61 (m, 1H), 1.77-1.87 (m, 1H), 1.99-2.17 (m, 5H), 2.18-2.23 (m, 1H), 2.26 (s, 3H), 2.35- 2.57 (m, 4H), 2.64-2.76 (m, 1H), 2.83-2.91 (m, 1H), 3.29 (s, 3H), 3.32-3.48 (m, 2H), 3.90-4.09 (m, 2H), 6.88-6.99 (m, 2H), 7.74-7.82 (m, 1H), 8.62 (brs, 1H) | 465 | 463 | Single isomer (Stereo- chemistry of Methoxy group: Undeter- mined) (Optical isomer of F-554) |
| F-556 | | (400 MHz, CDCl3) 0.81-0.90 (m, 7H), 0.90-0.98 (m, 1H), 1.08-1.18 (m, 2H), 1.33-1.39 (m, 2H), 1.49-1.61 (m, 1H), 1.97-2.10 (m, 3H), 2.10-2.13 (m, 3H), 2.23-2.27 (m, 3H), 2.34-2.46 (m, 4H), 2.46-2.57 (m, 2H), 2.83-2.94 (m, 1H), 3.38 (s, 3H), 3.40-3.49 (m, 1H), 3.60-3.71 (m, 2H), 4.00- 4.08 (m, 1H), 6.90-6.99 (m, 2H), 7.74-7.81 (m, 1H), 8.43 (brs, 1H) | 465 | 463 | Single isomer (Stereo- chemistry of Methoxy group: Undeter- mined) (Optical isomer of F-557) |
| F-557 | | (400 MHz, CDCl3) 0.82-0.90 (m, 7H), 0.90-0.98 (m, 1H), 1.08-1.16 (m, 2H), 1.32-1.39 (m, 2H), 1.49-1.62 (m, 1H), 1.93-2.10 (m, 3H), 2.12 (s, 3H), 2.25 (s, 3H), 2.35-2.45 (m, 4H), 2.45-2.54 (m, 2H), 2.85-2.94 (m, 1H), 3.38 (s, 3H), 3.40-3.49 (m, 1H), 3.58- 3.74 (m, 2H), 4.00-4.08 (m, 1H), 6.90-6.99 (m, 2H), 7.76- 7.83 (m, 1H), 8.44 (brs, 1H) | 465 | 463 | Single isomer (Stereo- chemistry of Methoxy group: Undeter- mined) (Optical isomer of F-556) |

TABLE 297

| | | | | | Infor- |
|---|---|---|---|---|---|
| | | | | | mation |
| Exam- | | | MS | | of |
| ple | Chemical structure | NMR | M + H | M − H | structure |
| F-558 | | (400 MHz, CDCl3) 0.86 (d, J = 6.62 Hz, 6H), 1.11-1.22 (m, 2H), 1.19 (d, J = 5.95 Hz, 3H), 1.24 (d, J = 5.95 Hz, 3H), 1.33-1.40 (m, 4H), 1.51-1.58 (m, 1H), 2.13-2.27 (m, 2H), 2.21 (s, 3H), 2.24 (s, 3H), 2.45-2.58 (m, 3H), 3.18-3.32 (m, 2H), 3.50-3.61 (m, 1H), 3.90-4.00 (m, 1H), 4.13 (brs, 1H), 5.57-5.65 (m, 1H), 6.89- 6.97 (m, 2H), 7.27-7.32 (m, 1H), 8.85 (brs, 1H) | 453 | 451 | |
| F-559 | | (400 MHz, DMSO-D6) 0.86 (s, 9H), 1.04 (s, 2H), 1.15 (t, J = 8.58 Hz, 2H), 1.34 (d, J = 6.49 Hz, 2H), 1.97-2.05 (m, 1H), 2.09 (s, 3H), 2.23 (s, 3H), 3.16 (s, 1H), 3.54-3.86 (m, 9H), 3.99 (dt, J = 15.61, 6.15 Hz, 2H), 4.17 (q, J = 7.96 Hz, 1H), 4.68 (q, J = 10.20 Hz, 2H), 6.95 (d, J = 8.12 Hz, 1H), 7.01 (s, 1H), 7.19 (d, J = 8.12 Hz, 1H), 9.61 (s, 1H) | 596 | 630 (Salt) | |
| F-560 | | (400 MHz, MeOH-D4) 0.88-0.92 (m, 7H), 1.16- 1.59 (m, 10H), 1.86- 2.13 (m, 4H), 2.10 (d, J = 6.40 Hz, 3H), 2.26 (s, 3H), 2.55-2.68 (m, 3H), 3.00-3.15 (m, 2H), 3.57-3.70 (m, 1H), 3.79-3.92 (m, 1H), 3.93-4.11 (m, 2H), 6.92-6.98 (m, 1H), 7.01-7.08 (m, 2H) | 453 | 451 | |

TABLE 298

| | | | | | Infor- |
|---|---|---|---|---|---|
| | | | | | mation |
| Exam- | | | MS | | of |
| ple | Chemical structure | NMR | M + H | M − H | structure |
| F-561 | | (400 MHz, DMSO-D6) 0.86 (s, 9H), 1.04-1.37 (m, 4H), 1.34 (d, J = 6.40 Hz, 2H), 1.81-2.08 (m, 4H), 2.05 (s, 3H), 2.22 (s, 3H), 2.44- 2.64 (m, 3H), 2.90-3.08 (m, 2H), 3.22-3.32 (m, 1H), 3.38-3.55 (m, 2H), 3.73-3.94 (m, 2H), 6.92 (d, J = 8.16 Hz, 1H), 6.98 (s, 1H), 7.09 (d, J = 8.16 Hz, 1H), 9.47 (brs, 1H) | 453 | 451 | |
| F-562 | | (400 MHz, CDCl3) 0.83-0.96 (m, 8H), 1.10-1.19 (m, 2H), 1.31-1.37 (m, 2H), 1.44 (s, 3H), 1.47-1.59 (m, 1H), 1.73- 1.83 (m, 1H), 2.02-2.17 (m, 6H), 2.24-2.33 (m, 4H), 2.36- 2.57 (m, 4H), 2.84-2.92 (m, 1H), 3.37-3.49 (m, 1H), 3.75- 3.85 (m, 1H), 4.05-4.14 (m, 2H), 6.92-7.00 (m, 2H), 7.61- 7.68 (m, 1H), 8.32-8.40 (m, 1H) | 465 | 463 | Single isomer (Stereo- chemistry of Hydroxyl group and Methyl group: Undeter- mined) |
| | | | | | (Optical |
| | | | | | isomer of |
| | | | | | F-563) |
| F-563 | | (400 MHz, CDCl3) 0.83-0.95 (m, 8H), 1.10-1.19 (m, 2H), 1.31-1.38 (m, 2H), 1.44 (s, 3H), 1.50-1.60 (m, 1H), 1.74- 1.83 (m, 1H), 2.02-2.17 (m, 4H), 2.23-2.33 (m, 6H), 2.33- 2.58 (m, 5H), 2.83-2.92 (m, 1H), 3.37-3.49 (m, 1H), 3.68- 3.84 (m, 1H), 3.97-4.14 (m, 2H), 6.93-7.00 (m, 2H), 7.63- 7.70 (m, 1H), 8.25-8.40 (m, 1H) | 465 | 463 | Single isomer (Stereo- chemistry of Hydroxyl group and Methyl group: Undeter- mined) |
| | | | | | (Optical |
| | | | | | isomer of |
| | | | | | F-562) |

TABLE 299

| | | | | | Infor- |
|---|---|---|---|---|---|
| | | | | | mation |
| Exam- | | | MS | | of |
| ple | Chemical structure | NMR | M + H | M − H | structure |
| F-564 | | (400 MHz, CDCl3) 0.78-0.88 (m, 9H), 0.88-0.97 (m, 1H), 1.06-1.20 (m, 2H), 1.24-1.32 (m, 2H), 1.45 (s, 3H), 1.46- 1.55 (m, 1H), 1.96-2.07 (m, 4H), 2.09-2.16 (m, 3H), 2.24-2.27 (m, 2H), 2.29-2.50 (m, 4H), 2.84-2.93 (m, 1H), 3.33-3.45 (m, 1H), 3.92-4.04 (m, 2H), 4.44-4.66 (m, 1H), 6.92-7.01 (m, 2H), 7.52-7.60 (m, 1H), 8.15-8.30 (m, 1H) | 465 | 463 | Single isomer (Stereo- chemistry of Hydroxyl group and Methyl group: Undeter- mined) |
| | | | | | (Optical |
| | | | | | isomer of |
| | | | | | F-565) |
| F-565 | | (400 MHz, CDCl3) 0.79-0.88 (m, 8H), 0.88-0.95 (m, 1H), 1.06-1.20 (m, 2H), 1.24- 1.30 (m, 2H), 1.45 (s, 3H), 1.47-1.54 (m, 1H), 1.97-2.06 (m, 2H), 2.12-2.20 (m, 4H), 2.26 (s, 3H), 2.28-2.54 (m, 6H), 2.84-2.92 (m, 1H), 3.34-3.46 (m, 1H), 3.89-4.05 (m, 1H), 4.34-4.61 (m, 1H), 6.92-7.00 (m, 2H), 7.53-7.59 (m, 1H), 8.09-8.22 (m, 1H) | 465 | 463 | Single isomer (Stereo- chemistry of Hydroxyl group and Methyl group: Undeter- mined) |
| | | | | | (Optical |
| | | | | | isomer of |
| | | | | | F-564) |
| F-566 | | (400 MHz, CDCl3) 0.79-0.84 (m, 7H), 1.12-1.19 (m, 3H), 1.29 (dd, J = 12.57, 5.73 Hz, 2H), 1.51 (td, J = 13.34, 6.69 Hz, 1H), 1.94-2.04 (m, 7H), 2.25 (s, 3H), 2.33-2.72 (m, 3H), 2.94 (dq, J = 18.69, 4.63 Hz, 2H), 3.16 (dt, J = 18.38, 6.89 Hz, 1H), 3.39 (dd, J = 17.20, 7.94 Hz, 1H), 4.11 (d, J = 5.51 Hz, 1H), 6.92 (d, J = 9.26 Hz, 2H), 7.25-7.28 (m, 2H) | 477 | 475 | |

TABLE 300

| | | | | | Infor- |
|---|---|---|---|---|---|
| | | | MS | | mation |
| Ex- | | | M + | M − | of |
| ample | Chemical structure | NMR | H | H | structure |
| F-567 | | (400 MHz, CDCl3) 0.77 (dd, J = 10.03, 4.74 Hz, 1H), 1.00 (t, J = 4.74 Hz, 6H), 1.27 (dd, J = 10.92, 5.84 Hz, 4H), 2.06-2.14 (m, 5H), 2.25 (s, 3H), 2.71 (d, J = 7.06 Hz, 2H), 3.00 (dd, J = 14.67, 4.74 Hz, 1H), 3.21-3.33 (m, 2H), 4.26 (d, J = 5.29 Hz, 1H), 6.55 (s, 1H), 6.94 (d, J = 6.62 Hz, 2H), 7.15 (s, 1H), 7.32 (t, J = 10.48 Hz, 1H) | 490 | 488 | |
| F-568 | | (400 MHz, DMSO-D6) 1.00-1.01 (m, 6H), 1.10 (d, J = 11.25 Hz, 1H), 1.33 (t, J = 7.06 Hz, 2H), 1.45 (t, J = 6.95 Hz, 3H), 1.64-1.70 (m, 1H), 2.09 (ddd, J = 30.32, 13.67, 7.94 Hz, 5H), 2.36 (d, J = 6.18 Hz, 3H), 2.60-2.62 (m, 4H), 3.05 (tt, J = 24.59, 8.23 Hz, 1H), 3.25 (dd, J = 15.77, 9.37 Hz, 1H), 3.34 (s, 1H), 3.87 (t, J = 8.60 Hz, 1H), 4.12-4.19 (m, 1H), 7.07 (d, J = 8.38 Hz, 1H), 7.13 (s, 1H), 7.24 (d, J = 8.16 Hz, 1H), 9.62 (s, 1H) | 477 | 475 | |
| F-569 | | (400 MHz, CDCl3) 0.93-0.98 (m, 1H), 1.01 (d, J = 6.62 Hz, 6H), 1.24 (dt, J = 19.41, 6.01 Hz, 6H), 2.12 (dd, J = 13.34, 6.73 Hz, 1H), 2.20 (s, 3H), 2.26 (s, 3H), 2.72 (d, J = 7.06 Hz, 2H), 3.23 (dd, J = 13.78, 4.96 Hz, 1H), 3.41 (dt, J = 13.82, 4.36 Hz, 2H), 3.63-3.69 (m, 1H), 3.77 (dt, J = 15.59, 7.00 Hz, 1H), 5.29 (dd, J = 8.38, 5.07 Hz, 1H), 6.60 (s, 1H), 6.97 (t, J = 7.17 Hz, 2H), 7.61 (d, J = 8.82 Hz, 1H), 8.07 (s, 1H) | 452 | 450 | |

TABLE 301

| | | | | | Infor- |
|---|---|---|---|---|---|
| | | | MS | | mation |
| Ex- | | | M + | M − | of |
| ample | Chemical structure | NMR | H | H | structure |
| F-570 | | (400 MHz, CDCl3) 0.85-0.88 (m, 1H), 1.01 (d, J = 6.62 Hz, 6H), 1.08 (d, J = 6.62 Hz, 1H), 1.33 (d, J = 7.50 Hz, 2H), 2.04-2.16 (m, 4H), 2.25 (s, 3H), 2.46 (t, J = 13.12 Hz, 1H), 2.72-2.87 (m, 5H), 3.36 (t, J = 3.64 Hz, 1H), 3.97- 4.02 (m, 1H), 4.20-4.25 (m, 1H), 6.61 (s, 1H), 6.94-6.95 (m, 2H), 7.62 (d, J = 8.82 Hz, 1H), 8.62 (s, 1H) | 484 | 482 | Racemic form |
| F-571 | | (400 MHz, CDCl3) 0.85 (d, J = 23.82 Hz, 6H), 1.09 (s, 1H), 1.30- 1.37 (m, 5H), 1.51-1.54 (m, 1H), 2.17 (s, 3H), 2.20-2.24 (m, 4H), 2.30 (d, J = 9.70 Hz, 1H), 2.46 (s, 1H), 2.58 (s, 3H), 2.80-2.83 (m, 3H), 3.29 (s, 1H), 3.62 (t, J = 10.00 Hz, 1H), 4.14 (q, J = 9.26 Hz, 1H), 4.24 (s, 1H), 6.92- 6.93 (m, 2H), 7.39 (d, J = 7.94 Hz, 1H), 8.97 (s, 1H) | 471 | 469 | |
| F-572 | | (400 MHz, CDCl3) 0.85 (d, J = 6.62 Hz, 6H), 1.11 (s, 1H), 1.30- 1.38 (m, 5H), 1.51-1.54 (m, 1H), 2.18-2.35 (m, 8H), 2.44-2.58 (m, 4H), 2.76-2.89 (m, 3H), 3.33 (s, 1H), 3.63 (t, J = 8.05 Hz, 1H), 4.15- 4.24 (m, 2H), 6.91-6.93 (m, 2H), 7.36 (t, J = 6.29 Hz, 1H), 9.09 (s, 1H) | 471 | 469 | |

TABLE 302

| | | | | | Infor- |
|---|---|---|---|---|---|
| | | | MS | | mation |
| Ex- | | | M + | M − | of |
| ample | Chemical structure | NMR | H | H | structure |
| F-573 | | (400 MHz, CDCl3) 0.91 (tt, J = 12.79, 4.52 Hz, 6H), 1.00-1.02 (m, 6H), 1.26 (dt, J = 13.45, 4.96 Hz, 2H), 1.85 (td, J = 13.34, 6.54 Hz, 2H), 2.12 (dd, J = 13.56, 6.73 Hz, 2H), 2.19 (s, 3H), 2.25 (s, 3H), 2.73 (d, J = 7.06 Hz, 2H), 3.22 (dd, J = 13.78, 4.74 Hz, 1H), 3.42 (dtd, J = 31.91, 11.83, 6.47 Hz, 4H), 5.29 (dd, J = 8.71, 4.74 Hz, 1H), 6.62 (d, J = 10.37 Hz, 1H), 6.97 (t, J = 8.49 Hz, 2H), 7.59 (t, J = 4.30 Hz, 1H), 8.10 (d, J = 10.37 Hz, 1H) | 480 | 478 | |
| F-574 | | (400 MHz, CDCl3) 0.85-1.60 (m, 8H), 0.85 (d, J = 6.40 Hz, 6H), 1.20 (d, J = 5.07 Hz, 3H), 1.83-2.12 (m, 4H), 2.07 (d, J = 18.53 Hz, 3H), 2.25 (d, J = 2.21 Hz, 3H), 2.37-2.50 (m, 3H), 2.79-2.95 (m, 2H), 3.01- 3.14 (m, 1H), 3.36-3.46 (m, 1H), 3.54-4.05 (m, 2H), 6.91-6.96 (m, 2H), 7.38-7.52 (m, 2H), 7.83 (s, 1H) | 453 | 451 | |
| F-575 | | (400 MHz, MeOH-D4) 1.02 (d, J = 6.62 Hz, 6H), 1.11-1.26 (m, 2H), 1.24 (d, J = 6.18 Hz, 3H), 1.28- 1.36 (m, 2H), 1.30 (d, J = 6.18 Hz, 3H), 2.07-2.19 (m, 1H), 2.18 (s, 3H), 2.28 (s, 3H), 2.82 (d, J = 7.06 Hz, 2H), 3.21-3.26 (m, 2H), 3.63-3.69 (m, 1H), 3.89- 3.99 (m, 1H), 5.60 (t, J = 5.95 Hz, 1H), 6.75-6.81 (m, 1H), 6.98 (d, J = 8.16 Hz, 1H), 7.05 (s, 1H), 7.12 (d, J = 7.94 Hz, 1H) | 466 | 464 | |

TABLE 303

| | | | | | Infor- |
|---|---|---|---|---|---|
| | | | MS | | mation |
| Ex- | | | M + | M − | of |
| ample | Chemical structure | NMR | H | H | structure |
| F-576 | | (400 MHz, MeOH-D4) 1.02 (d, J = 6.62 Hz, 6H), 1.12-1.38 (m, 4H), 1.24 (d, J = 5.95 Hz, 3H), 1.30 (d, J = 5.95 Hz, 3H), 2.08-2.16 (m, 1H), 2.23 (s, 3H), 2.82 (d, J = 7.06 Hz, 2H), 3.21-3.26 (m, 2H), 3.57-3.69 (m, 1H), 3.86- 3.99 (m, 1H), 5.60 (t, J = 5.95 Hz, 1H), 6.79 (s, 1H), 6.90 (td, J = 8.38, 2.72 Hz, 1H), 6.99 (dd, J = 9.81, 2.76 Hz, 1H), 7.24 (dd, J = 8.71, 5.62 Hz, 1H) | 470 | 468 | |
| F-577 | | (400 MHz, DMSO-D6) 0.81-0.97 (m, 2H), 0.95 (d, J = 6.62 Hz, 6H), 1.13-1.24 (m, 2H), 1.99-2.06 (m, 1H), 2.00 (d, J = 13.01 Hz, 3H), 2.06 (s, 3H), 2.23 (s, 3H), 2.62 (d, J = 7.06 Hz, 2H), 3.41-3.52 (m, 2H), 3.65-3.93 (m, 2H), 3.97- 4.25 (m, 3H), 4.95 (brs, 1H), 6.94 (d, J = 8.16 Hz, 1H), 7.00 (s, 1H), 7.05 (d, J = 3.97 Hz, 1H), 7.20 (dd, J = 11.36, 8.05 Hz, 1H), 9.58 (d, J = 7.50 Hz, 1H) | 491 | 489 | |
| F-578 | | (400 MHz, CDCl3) 0.35-0.53 (m, 2H), 0.77-0.92 (m, 2H), 0.87 (d, J = 6.68 Hz, 6H), 1.29-1.44 (m, 3H), 1.48-1.61 (m, 1H), 1.86- 2.45 (m, 8H), 2.08 (s, 3H), 2.26 (s, 3H), 2.81 (dd, J = 14.24, 6.80 Hz, 1H), 2.99 (dd, J = 14.24, 8.68 Hz, 1H), 3.43-3.57 (m, 1H), 3.65- 3.82 (m, 3H), 6.93-7.04 (m, 2H), 7.40 (s, 1H), 7.51 (d, J = 8.68 Hz, 1H) | 439 | 437 | |

TABLE 304

| | | | | | Infor- |
|---|---|---|---|---|---|
| | | | | | mation |
| Ex- | | | MS | | of |
| ample | Chemical structure | NMR | M + H | M − H | structure |
| F-579 | | (400 MHz, CDCl3) 0.33-0.51 (m, 2H), 0.77-0.96 (m, 2H), 0.88 (d, J = 6.44 Hz, 6H), 1.31-1.47 (m, 3H), 1.50-1.68 (m, 1H), 1.87- 2.63 (m, 8H), 2.09 (s, 3H), 2.26 (s, 3H), 2.82 (dd, J = 14.24. 5.64 Hz, 1H), 3.01 (dd, J = 14.24, 8.92 Hz, 1H), 3.58-3.83 (m, 4H), 6.92- 7.04 (m, 2H), 7.42 (s, 1H), 7.52 (d, J = 8.24 Hz, 1H) | 439 | 437 | |
| F-580 | | (400 MHz, CDCl3) 0.35-0.52 (m, 2H), 0.78-0.93 (m, 8H), 1.30- 1.46 (m, 3H), 1.49-1.68 (m, 1H), 1.85-2.65 (m, 11H), 2.26 (s, 3H), 2.75-2.88 (m, 1H), 2.94-3.08 (m, 1H), 3.45-3.56 (m, 0.5H), 3.59- 3.83 (m, 3.5H), 6.93-7.04 (m, 2H), 7.35-7.46 (m, 1H), 7.48-7.57 (m, 1H) | 439 | 437 | Mixtures of four stereo- isomers |
| F-581 | | (400 MHz, CDCl3) 0.86 (d, J = 6.62 Hz, 6H), 0.89-0.95 (m, 4H), 0.95-1.08 (m, 1H), 1.34 (t, J = 7.06 Hz, 2H), 1.47-1.58 (m, 1H), 1.99-2.11 (m, 2H), 2.14 (s, 3H), 2.25 (s, 3H), 2.31-2.55 (m, 3H), 2.77-2.87 (m, 1H), 3.28 (dd, J = 13.56, 4.52 Hz, 1H), 3.35-3.53 (m, 2H), 4.62 (d, J = 11.47 Hz, 1H), 4.72 (d, J = 11.47 Hz, 1H), 5.28 (dd, J = 8.71, 4.52 Hz, 1H), 6.90-6.99 (m, 2H), 7.27-7.35 (m, 5H), 7.59 (d, J = 8.82 Hz, 1H), 8.40 (brs, 1H) | 501 | 499 | |

TABLE 305

| | | | | | Infor- |
|---|---|---|---|---|---|
| | | | MS | | mation |
| Ex- | | | M + | M − | of |
| ample | Chemical structure | NMR | H | H | structure |
| F-582 | | (400 MHz, CDCl3) 0.78-1.05 (m, 14H), 1.13 (ddd, J = 15.93, 8.99, 4.91 Hz, 2H), 1.35 (q, J = 7.20 Hz, 2H), 1.51 (tt, J = 17.97, 6.32 Hz, 1H), 1.76-1.86 (m, 1H), 2.01- 2.16 (m, 5H), 2.25 (s, 3H), 2.36- 2.53 (m, 3H), 2.96-3.01 (m, 1H), 3.15 (dt, J = 18.01, 6.23 Hz, 1H), 3.29-3.33 (m, 1H), 3.37-3.52 (m, 3H), 5.11 (dd, J = 9.04, 4.41 Hz, 1H), 6.96 (t, J = 10.14 Hz, 2H), 7.53- 7.57 (m, 1H), 8.50 (s, 1H) | 467 | 465 | |
| F-583 | | (400 MHz, CDCl3) 0.74-0.81 (m, 1H), 0.82-0.88 (m, 7H), 1.07- 1.16 (m, 2H), 1.27-1.35 (m, 2H), 1.47-1.58 (m, 2H), 1.85-1.94 (m, 2H), 1.98-2.02 (m, 3H), 2.02- 2.12 (m, 1H), 2.21-2.24 (m, 4H), 2.31-2.35 (m, 4H), 2.35-2.52 (m, 2H), 2.80-2.88 (m, 1H), 2.93- 3.06 (m, 4H), 3.32-3.43 (m, 1H), 3.58-3.70 (m, 1H), 6.86-6.94 (m, 2H), 7.31-7.36 (m, 1H), 7.43- 7.50 (m, 1H) | 464 | 462 | Racemic form |
| F-584 | | (400 MHz, DMSO-D6) 0.73-0.80 (m, 1H), 0.95 (d, J = 6.72 Hz, 6H), 1.07-1.26 (m, 9H), 1.96-2.13 (m, 1H), 2.76 (d, J = 7.19 Hz, 2H), 3.14- 3.29 (m, 2H), 3.44-3.48 (m, 1H), 3.72-3.81 (m, 1H), 5.40 (t, J = 6.84 Hz, 1H), 6.80 (s, 1H), 7.12- 7.17 (m, 2H), 7.41-7.47 (m, 1H), 10.39 (s, 1H) | 518 | 516 | |

TABLE 306

| | | | | | Infor- |
|---|---|---|---|---|---|
| | | | MS | | mation |
| Ex- | | | M + | M − | of |
| ample | Chemical structure | NMR | H | H | structure |
| F-585 | | (400 MHz, DMSO-D6) 0.71-0.81 (m, 1H), 0.95 (d, J = 6.49 Hz, 6H), 1.05-1.26 (m, 9H), 2.76 (d, J = 6.96 Hz, 2H), 3.15 (d, J = 6.96 Hz, 2H), 3.43-3.50 (m, 1H), 3.73- 3.79 (m, 1H), 5.40 (t, J = 7.07 Hz, 1H), 6.79 (s, 1H), 7.23 (dd, J = 8.70, 1.97 Hz, 1H), 7.34 (d, J = 8.81 Hz, 1H), 7.74 (d, J = 2.09 Hz, 1H), 10.41 (s, 1H) | 518 | 516 | |
| F-586 | | (400 MHz, CDCl3) 0.69-0.74 (m, 1H), 1.00 (dt, J = 17.64, 7.94 Hz, 13H), 1.23-1.30 (m, 3H), 1.65- 1.67 (m, 2H), 2.07-2.13 (m, 4H), 2.24 (s, 3H), 2.71 (d, J = 7.06 Hz, 2H), 2.88 (dd, J = 14.11, 3.97 Hz, 1H), 3.14 (dd, J = 13.89, 10.81 Hz, 1H), 3.25-3.31 (m, 1H), 3.91 (dd, J = 10.70, 4.08 Hz, 1H), 6.55 (s, 1H), 6.93 (d, J = 5.73 Hz, 2H), 7.42 (d, J = 8.60 Hz, 1H), 7.54 (d, J = 10.37 Hz, 1H) | 464 | 462 | |
| F-587 | | (400 MHz, DMSO-D6) 1.02-1.34 (m, 4H), 1.78-1.98 (m, 2H), 2.05 (s, 3H), 2.11-2.48 (m, 2H), 2.22 (s, 3H), 2.28 (d, J = 5.29 Hz, 2H), 2.54-2.74 (m, 3H), 2.90-3.08 (m, 2H), 3.21-3.31 (m, 1H), 3.33- 3.54 (m, 2H), 3.73-3.94 (m, 2H), 6.92 (d, J = 8.60 Hz, 1H), 6.98 (s, 1H), 7.10 (d, J = 8.60 Hz, 1H), 9.46 (brs, 1H) | 465 | 463 | |
| F-588 | | (400 MHz, DMSO-D6) 0.78-1.35 (m, 4H), 1.76-2.74 (m, 9H), 2.10 (s, 3H), 2.88-3.09 (m, 2H), 3.12- 4.20 (m, 5H), 6.90-7.38 (m, 3H), 9.57 (brs, 1H) | 469 | 467 | |

TABLE 307

| | | | | | Infor- |
|---|---|---|---|---|---|
| | | | MS | | mation |
| Ex- | | | M + | M − | of |
| ample | Chemical structure | NMR | H | H | structure |
| F-589 | | (400 MHz, DMSO-D6) 0.77-1.35 (m, 4H), 1.77-2.76 (m, 9H), 2.06 (s, 3H), 2.90-3.11 (m, 2H), 3.21- 3.56 (m, 3H), 3.73-4.45 (m, 2H), 6.59-7.31 (m, 3H), 9.77 (s, 1H) | 469 | 467 | |
| F-590 | | (400 MHz, CDCl3) 0.86 (d, J = 6.62 Hz, 6H), 0.95-1.73 (m, 17H), 1.97-2.09 (m, 5H), 2.24 (s, 3H), 2.33-2.51 (m, 3H), 2.77- 2.88 (m, 2H), 3.04 (ddd, J = 15.77, 8.82, 4.85 Hz, 1H), 3.36-3.45 (m, 1H), 3.73 (dd, J = 10.92, 4.08 Hz, 1H), 6.92 (d, J = 7.06 Hz, 2H), 7.38 (t, J = 6.06 Hz, 1H), 7.70 (s, 1H) | 451 | 449 | |
| F-591 | | (400 MHz, CDCl3) 0.71 (brs, 1H), 1.00 (t, J = 1.98 Hz, 12H), 1.24 (brs, 4H), 1.65 (d, J = 17.42 Hz, 2H), 2.00-2.09 (m, 4H), 2.25 (s, 3H), 2.63 (dd, J = 7.06, 1.32 Hz, 2H), 2.89 (dd, J = 14.11, 3.31 Hz, 1H), 3.13 (t, J = 12.46 Hz, 1H), 3.33 (brs, 1H), 3.90 (brs, 1H), 6.70 (s, 1H), 6.94 (d, J = 6.18 Hz, 2H), 7.41 (t, J = 10.59 Hz, 2H) | 464 | 462 | |
| F-592 | | (400 MHz, CDCl3) 0.99-1.01 (m, 7H), 1.23 (s, 1H), 1.41 (d, J = 7.50 Hz, 2H), 2.07-2.14 (m, 1H), 2.21 (s, 3H), 2.71-2.74 (m, 4H), 2.93 (s, 2H), 3.53 (s, 1H), 4.28 (q, J = 8.16 Hz, 1H), 4.44 (q, J = 9.92 Hz, 1H), 6.69 (s, 1H), 6.79- 6.86 (m, 2H), 7.49 (dd, J = 8.71, 5.40 Hz, 1H), 9.14 (s, 1H) | 488 | 486 | |

TABLE 308

| | | | | | Infor- |
|---|---|---|---|---|---|
| | | | | | mation |
| Ex- | | | MS | | of |
| ample | Chemical structure | NMR | M + H | M − H | structure |
| F-593 | | (400 MHz, CDCl3) 1.02-1.06 (m, 7H), 1.24 (s, 1H), 1.42 (d, J = 7.50 Hz, 2H), 2.07-2.14 (m, 1H), 2.20 (d, J = 3.31 Hz, 3H), 2.73- 2.75 (m, 4H), 2.87-2.95 (m, 2H), 3.55 (s, 1H), 4.32 (q, J = 9.59 Hz, 1H), 4.46 (q, J = 9.92 Hz, 1H), 6.70 (s, 1H), 6.80-6.84 (m, 2H), 7.47 (dd, J = 8.71, 5.40 Hz, 1H), 9.16 (s, 1H) | 488 | 486 | |
| F-594 | | (400 MHz, CDCl3) 0.85-0.88 (m, 7H), 1.09-1.61 (m, 7H), 2.03- 2.19 (m, 7H), 2.32-2.62 (m, 4H), 3.01-3.09 (m, 1H), 3.33-3.60 (m, 1H), 3.77-3.80 (m, 2H), 4.14 (s, 1H), 6.77-6.89 (m, 3H), 8.99 (d, J = 12.35 Hz, 1H) | 443 | 441 | |
| F-595 | | (400 MHz, DMSO-D6) 0.61-0.67 (m, 1H), 0.90 (dt, J = 21.80, 7.88 Hz, 12H), 1.07-1.17 (m, 3H), 1.54-1.61 (m, 2H), 1.64-1.72 (m, 1H), 1.99-2.06 (m, 4H), 2.20 (s, 3H), 2.79 (dt, J = 22.03, 7.77 Hz, 3H), 2.97 (dd, J = 15.19, 9.16 Hz, 1H), 3.38 (ddd, J = 14.32, 7.13, 3.77 Hz, 2H), 3.77 (t, J = 7.54 Hz, 1H), 4.29 (s, 1H), 6.77 (s, 1H), 6.90 (d, J = 8.35 Hz, 1H), 6.95 (s, 1H), 7.09 (d, J = 8.12 Hz, 1H), 9.36 (s, 1H) | 464 | 462 | |

TABLE 309

| | | | | | Infor- |
|---|---|---|---|---|---|
| | | | MS | | mation |
| Ex- | | | M + | M − | of |
| ample | Chemical structure | NMR | H | H | structure |
| F-596 | | (400 MHz, CDCl3) 0.81-0.89 (m, 8H), 1.07-1.19 (m, 8H), 1.23- 1.32 (m, 1H), 1.35-1.43 (m, 1H), 2.20-2.25 (m, 4H), 2.25-2.28 (m, 3H), 2.39-2.52 (m, 1H), 2.52- 2.65 (m, 3H), 3.07-3.16 (m, 3H), 3.52-3.60 (m, 1H), 3 63-3.73 (m, 1H), 4.05-4.16 (m, 1H), 4.31- 4.43 (m, 1H), 4.61-4.76 (m, 2H), 4.79-4.97 (m, 2H), 6.87-6.98 (m, 3H), 7.29-7.33 (m, 1H), 10.17 (brs, 1H) | 492 | 490 | |
| F-597 | | (400 MHz, CDCl3) 0.78-0.89 (m, 12H), 1.16-1.30 (m, 1H), 1.46- 1.57 (m, 3H), 2.11-2.23 (m, 4H), 2.23-2.29 (m, 3H), 2.37-2.63 (m, 4H), 3.29-3.43 (m, 1H), 3.52- 3.75 (m, 4H), 4.13-4.31 (m, 2H), 4.47-4.60 (m, 4H), 4.80-4.90 (m, 1H), 6.85-6.97 (m, 3H), 7.28- 7.31 (m, 1H), 10.19 (brs, 1H), 12.22-12.44 (m, 1H) | 478 | 476 | |
| F-598 | | (400 MHz, DMSO-D6) 0.85 (d, J = 6.68 Hz, 6H), 1.06-1.35 (m, 12H), 1.35-1.58 (m, 2H), 1.77- 2.08 (m, 4H), 2.04 (s, 3H), 2.22 (s, 3H), 2.37-2.63 (m, 3H), 2.94- 4.00 (m, 5H), 6.92 (d, J = 8.00 Hz, 1H), 6.98 (s, 1H), 7.08 (d, J = 8.00 Hz, 1H), 9.49 (s, 1H) | 467 | 465 | |
| F-599 | | (400 MHz, DMSO-D6) 0.85 (d, J = 6.68 Hz, 6H), 1.06-1.35 (m, 12H), 1.38-1.58 (m, 2H), 1.77- 2.08 (m, 4H), 2.04 (s, 3H), 2.22 (s, 3H), 2.37-2.63 (m, 3H), 2.94- 4.00 (m, 5H), 6.92 (d, J = 8.00 Hz, 1H), 6.98 (s, 1H), 7.08 (d, J = 8.00 Hz, 1H), 9.49 (s, 1H) | 467 | 465 | |

TABLE 310

| | | | | | Infor- |
|---|---|---|---|---|---|
| | | | MS | | mation |
| Ex- | | | M + | M − | of |
| ample | Chemical structure | NMR | H | H | structure |
| F-600 | | (400 MHz, DMSO-D6) 0.85 (d, J = 6.80 Hz, 6H), 1.08-1.23 (m, 4H), 1.31 (t, J = 7.19 Hz, 2H), 1.42- 1.55 (m, 2H), 1.97-2.07 (m, 4H), 2.13-2.15 (m, 3H), 2.41-2.54 (m, 3H), 3.24-4.20 (m, 10H), 6.95- 7.01 (m, 1H), 7.08 (dd, J = 9.62, 2.90 Hz, 1H), 7.28-7.34 (m, 1H), 9.74 (d, J = 12.29 Hz, 1H) | 482 | 480 | |
| F-601 | | (400 MHz, DMSO-D6) 0.84 (dt, J = 16.47, 6.29 Hz, 8H), 0.92 (dd, J = 9.04, 5.95 Hz, 6H), 1.02 (dd, J = 7.50, 4.63 Hz, 1H), 1.17-1.25 (m, 3H), 1.32 (t, J = 7.06 Hz, 3H), 1.42-1.70 (m, 3H), 1.93-1.96 (m, 1H), 2.06 (s, 3H), 2.21 (s, 3H), 2.45-2.53 (m, 2H), 2.95 (ddd, J = 27.35, 15.77, 7.28 Hz, 2H), 3.31- 3.38 (brm, 2H), 3.76 (t, J = 8.27 Hz, 2H), 6.92 (d, J = 8.16 Hz, 1H), 6.99 (d, J = 11.91 Hz, 1H), 7.10 (d, J = 7.94 Hz, 1H), 9.44 (s, 1H) | 451 | 449 | |
| F-602 | | (400 MHz, CDCl3) 0.84-0.91 (m, 10H), 1.09-1.18 (m, 2H), 1.21- 1.30 (m, 4H), 1.41-1.57 (m, 2H), 1.56-1.68 (m, 2H), 2.05-2.18 (m, 7H), 2.18-2.33 (m, 6H), 2.91- 3.05 (m, 1H), 3.18-3.35 (m, 2H), 3.58-3.66 (m, 1H), 3.73-3.89 (m, 1H), 3.97-4.24 (m, 2H), 6.88- 6.98 (m, 2H), 7.19-7.25 (m, 1H), 8.50-8.64 (m, 1H) | 479 | 477 | |

TABLE 311

| | | | | | Infor- |
|---|---|---|---|---|---|
| | | | MS | | mation |
| Ex- | | | M + | M − | of |
| ample | Chemical structure | NMR | H | H | structure |
| F-603 | | (400 MHz, DMSO-D6) 0.85 (d, J = 6.80 Hz, 6H), 1.05-1.23 (m, 4H), 1.30 (t, J = 6.84 Hz, 2H), 1.49- 1.55 (m, 2H), 1.97-2.07 (m, 4H), 2.14-2.15 (m, 3H), 2.41-2.55 (m, 3H), 3.20-4.17 (m, 9H), 7.21 (dd, J = 8.46, 2.67 Hz, 1H), 7.29 (s, 1H), 7.40 (dd, J = 13.33, 8.46 Hz, 1H), 9.74 (d, J = 10.44 Hz, 1H) | 498 | 496 | |
| F-604 | | (400 MHz, DMSO-D6) 0.84 (d, J = 6.40 Hz, 6H), 1.04-1.17 (m, 4H), 1.29 (t, J = 6.72 Hz, 2H), 1.48- 1.55 (m, 2H), 1.97-1.99 (m, 4H), 2.32-2.50 (m, 3H), 3.17-4.17 (m, 9H), 7.25 (d, J = 8.58 Hz, 1H), 7.47- 7.51 (m, 1H), 7.66-7.94 (m, 1H), 10.21 (d, J = 16.70 Hz, 1H) | 502 | 500 | |
| F-605 | | (400 MHz, DMSO-D6) 0.66 (t, J = 5.29 Hz, 1H), 0.95 (d, J = 6.62 Hz, 6H), 1.09-1.19 (m, 5H), 1.98 (s, 3H), 2.06 (td, J = 13.56, 6.84 Hz, 2H), 2.20 (d, J = 4.85 Hz, 3H), 2.76 (d, J = 7.06 Hz, 2H), 2.89 (tt, J = 22.39, 8.34 Hz, 3H), 3.14 (dd, J = 15.77, 9.37 Hz, 1H), 3.39 (dt, J = 12.28, 4.52 Hz, 1H), 4.03 (q, J = 7.13 Hz, 1H), 6.91 (d, J = 8.16 Hz, 1H), 6.96 (s, 1H), 7.08 (d, J = 7.94 Hz, 1H), 9.44 (s, 1H) | 490 | 488 | |
| F-606 | | (400 MHz, CDCl3) 0.98-1.05 (m, 14H), 1.42 (s, 2H), 1.68-1.70 (m, 3H), 2.10 (q, J = 6.73 Hz, 1H), 2.25 (s, 3H), 2.75 (d, J = 7.06 Hz, 2H), 3.19 (d, J = 10.00 Hz, 1H), 3.48 (s, 1H), 4.15 (s, 2H), 6.60 (s, 1H), 7.06 (d, J = 8.60 Hz, 1H), 7.11 (d, J = 2.43 Hz, 1H), 7.43 (d, J = 8.60 Hz, 1H), 8.92 (s, 1H) | 484 | 482 | |

TABLE 312

| | | | | | Infor- |
|---|---|---|---|---|---|
| | | | MS | | mation |
| Ex- | | | M + | M − | of |
| ample | Chemical structure | NMR | H | H | structure |
| F-607 | | (400 MHz, CDCl3) 0.92-1.01 (m, 14H), 1.43 (t, J = 3.20 Hz, 2H), 1.69- 1.78 (m, 3H), 2.07-2.14 (m, 1H), 2.24 (s, 3H), 2.75 (d, J = 7.06 Hz, 2H), 3.18 (d, J = 7.00 Hz, 1H), 3.50 (s, 1H), 4.17-4.19 (m, 2H), 6.61 (s, 1H), 6.76-6.85 (m, 2H), 7.35- 7.37 (m, 1H), 8.94 (s, 1H) | 468 | 466 | |
| F-608 | | (400 MHz, CDCl3) 0.92-1.05 (m, 1H), 1.02 (s, 9H), 1.32-1.49 (m, 2H), 1.55-1.65 (m, 1H), 2.15- 2.34 (m, 2H), 2.26 (s, 3H), 2.75 (s, 2H), 3.03-3.14 (m, 1H), 3.55- 3.66 (m, 1H), 3.76-3.91 (m, 2H), 4.13-4.25 (m, 1H), 4.27-4.39 (m, 1H), 6.61 (s, 1H), 7.08 (d, J = 8.68, 2.44 Hz, 1H), 7.13 (d, J = 2.20 Hz, 1H), 7.48 (d, J = 8.68 Hz, 1H), 8.61 (s, 1H) | 486 | 484 | |
| F-609 | | (400 MHz, CDCl3) 0.86 (d, J = 6.62 Hz, 6H), 1.20-1.46 (m, 6H), 1.50-1.57 (m, 1H), 2.19- 2.36 (m, 2H), 2.21 (s, 3H), 2.24 (s, 3H), 2.41-2.54 (m, 1H), 2.54- 2.67 (m, 2H), 3.37-3.47 (m, 2H), 3.58-3.68 (m, 1H), 3.93-4.03 (m, 1H), 4.06-4.16 (m, 2H), 5.72- 5.78 (m, 1H), 6.88-6.96 (m, 2H), 7.24-7.29 (m, 1H), 8.88 (brs, 1H) | 493 | 491 | |

TABLE 313

| | | | | | Infor- |
|---|---|---|---|---|---|
| | | | MS | | mation |
| Ex- | | | M + | M − | of |
| ample | Chemical structure | NMR | H | H | structure |
| F-610 | | (400 MHz, CDCl3) 1.00-1.09 (m, 2H), 1.12-1.23 (m, 2H), 1.57- 1.68 (m, 2H), 1.73-1.84 (m, 4H), 1.88-1.96 (m, 2H), 1.97-2.03 (m, 3H), 2.13-2.18 (m, 3H), 2.18- 2.25 (m, 1H), 2.27-2.36 (m, 2H), 3.15-3.23 (m, 2H), 3.59-3.93 (m, 4H), 3.96-4.10 (m, 2H), 4.10- 4.20 (m, 1H), 7.17-7.24 (m, 1H), 7.31 (brs, 1H), 7.37-7.46 (m, 1H), 9.69-9.77 (m, 1H) | 546 | 544 | |
| F-611 | | (400 MHz, DMSO-D6) 1.08-1.32 (m, 11H), 1.77 (dd, J = 20.29, 5.07 Hz, 2H), 1.94-2.05 (m, 4H), 2.13-2.22 (m, 6H), 2.57-2.59 (brm, 1H), 3.27 (brs, 1H), 3.48 (dd, J = 19.41, 9.26 Hz, 1H), 3.94 (ttt, J = 83.81, 28.56, 9.03 Hz, 8H), 7.22 (dd, J = 8.49, 2.32 Hz, 1H), 7.30 (s, 1H), 7.40 (dd, J = 13.01, 8.60 Hz, 1H), 9.81 (d, J = 13.89 Hz, 1H) | 516 | 514 | |
| F-612 | | (400 MHz, CDCl3) 0.82 (s, 1H), 1.00 (s, 9H), 1.15-1.21 (m, 3H) 2.11-2.19 (m, 8H), 2.70 (s, 2H), 2.99 (dd, J = 7.28, 3.64 Hz, 1H), 3.18 (dd, J = 12.13, 6.06 Hz, 1H), 3.34 (s, 2H), 3.71-3.73 (m, 2H), 4.12 (s, 1H), 6.48 (s, 1H), 6.91- 6.93 (m, 2H), 7.32 (d, J = 7.72 Hz, 1H), 8.12 (s, 1H) | 466 | 464 | |

TABLE 314

| | | | | | Infor- |
|---|---|---|---|---|---|
| | | | MS | | mation |
| Ex- | | | M + | M − | of |
| ample | Chemical structure | NMR | H | H | structure |
| F-613 | | (400 MHz, DMSO-D6) 0.80-0.89 (m, 1H), 0.95 (d, J = 6.80 Hz, 6H), 1.07-1.16 (m, 2H), 1.99-2.13 (m, 8H), 2.76 (d, J = 7.06 Hz, 2H), 3.37- 4.22 (m, 8H), 6.77 (d, J = 1.20 Hz, 1H), 7.20-7.22 (m, 1H), 7.29 (d, J = 2.21 Hz, 1H), 7.42 (dd, J = 11.80, 8.71 Hz, 1H), 9.72 (d, J = 8.16 Hz, 1H) | 511 | 509 | |
| F-614 | | (400 MHz, CDCl3) 1.01 (d, J = 6.84 Hz, 6H), 1.04-1.13 (m, 1H), 1.22-1.29 (m, 6H), 1.41- 1.53 (m, 3H), 2.05-2.18 (m, 1H), 2.29 (s, 3H), 2.76 (d, J = 7.06 Hz, 2H), 3.37 (dd, J = 14.89, 4.52 Hz, 1H), 3.51-3.66 (m, 1H), 3.96- 4.02 (m, 1H), 4.18-4.28 (m, 1H), 5.73 (dd, J = 9.92, 4.63 Hz, 1H), 6.70 (s, 1H), 7.05-7.10 (m, 1H), 7.11-7.15 (m, 1H), 7.51 (d, J = 8.60 Hz, 1H), 9.04 (s, 1H) | 486 | 484 | |
| F-615 | | (400 MHz, CDCl3) 0.98-1.04 (m, 7H), 1.33-1.36 (m, 2H), 1.58- 1.63 (m, 4H), 2.20-2.31 (m, 8H), 2.83-2.89 (m, 2H), 3.17 (d, J = 11 47 Hz, 1H), 3.69-3.79 (m, 3H), 3.93-3.96 (m, 1H), 4.27 (s, 1H), 6.60 (s, 1H), 6.90-6.92 (m, 2H), 7.24 (d, J = 8.38 Hz, 1H), 8.69 (s, 1H) | 466 | 464 | |
| F-616 | | (400 MHz, CDCl3) 0.97-1.01 (m, 7H), 1.36 (dd, J = 13.23, 6.84 Hz, 2H), 1.54-1.65 (m, 4H), 2.20- 2.23 (m, 5H), 2.84 (t, J = 7.61 Hz, 2H), 3.25 (d, J = 16.10 Hz, 1H), 3.75- 3.88 (m, 4H), 4.25 (s, 1H), 6.58 (s, 1H), 7.05-7.08 (m, 2H), 7.36 (d, J = 8.38 Hz, 1H), 9.01 (s, 1H) | 486 | 484 | |

TABLE 315

| | | | MS | | Information of |
|---|---|---|---|---|---|
| Example | Chemical structure | NMR | M + H | M − H | structure |
| F-617 | | (400 MHz, CDCl3) 0.84 (d, J = 6.68 Hz, 6H), 0.91-1.02 (m, 1H), 1.08-1.30 (m, 5H), 1.43- 1.58 (m, 1H), 1.87-2.11 (m, 2H), 2.01 (s, 3H), 2.24 (s, 3H), 2.31- 2.57 (m, 3H), 2.99-3.10 (m, 1H), 3.15-3.27 (m, 1H), 3.37-3.57 (m, 2H), 3.83-3.99 (m, 2H), 4.48- 4.63 (m, 1H), 4.63-4.75 (m, 1H), 6.89-6.98 (m, 2H), 7.21 (d, J = 8.00 Hz, 1H), 7.88-8.02 (m, 1H) | 475 | 473 | Racemic form |
| F-618 | | (400 MHz, CDCl3) 0.77-0.89 (m, 2H), 0.89-1.00 (m, 8H), 1.02- 1.17 (m, 2H), 1.99-2.10 (m, 2H), 2.10-2.13 (m, 3H), 2.22-2.27 (m, 3H), 2.74-2.80 (m, 1H), 2.97- 3.03 (m, 1H), 3.33-3.44 (m, 1H), 3.55-3.59 (m, 3H), 3.86-4.00 (m, 2H), 3.99-4.20 (m, 1H), 4.23- 4.48 (m, 1H), 6.77-6.81 (m, 1H), 6.94-7.01 (m, 1H), 7.01-7.04 (m, 1H), 7.23-7.30 (m, 1H) | 463 | 461 | |
| F-619 | | (400 MHz, CDCl3) 1.03-1.29 (m, 4H), 1.55-1.67 (m, 2H), 1.73- 1.85 (m, 4H), 1.83-1.98 (m, 4H), 2.00-2.06 (m, 3H), 2.07-2.19 (m, 2H), 2.18-2.24 (m, 3H), 2.27- 2.37 (m, 2H), 2.87-3.03 (m, 2H), 3.11-3.25 (m, 1H), 3.28-3.54 (m, 4H), 3.80-3.93 (m, 2H), 6.87- 6.95 (m, 1H), 6.97 (brs, 1H), 7.07- 7.13 (m, 1H), 9.41 (brs, 1H) | 487 | 485 | |

TABLE 316

| | | | MS | | Information of |
|---|---|---|---|---|---|
| Example | Chemical structure | NMR | M + H | M − H | structure |
| F-620 | | (400 MHz, CDCl3) 0.69-1.02 (m, 2H), 1.15-1.23 (m, 2H), 1.69- 1.97 (m, 8H), 2.13-2.16 (m, 6H), 2.23-2.35 (m, 5H), 2.88-2.97 (m, 2H), 3.25-3.36 (m, 1H), 3.55- 3.63 (m, 1H), 3.64-3.74 (m, 1H), 3.86-3.95 (m, 1H), 4.17-4.27 (m, 3H), 4.40-4.47 (m, 1H), 6.90- 6.99 (m, 2H), 7.71-7.76 (m, 1H), 8.89 (brs, 1H) | 526 | 524 | |
| F-621 | | (400 MHz, CDCl3) 0.85 (d, J = 6.40 Hz, 6H), 0.99-1.08 (m, 1H), 1.23-1.43 (m, 4H), 1.47- 1.57 (m, 2H), 2.01-2.26 (m, 5H), 2.14 (s, 3H), 2.24 (s, 3H), 2.40- 2.49 (m, 1H), 2.50-2.62 (m, 2H), 3.16 (d, J = 15.66 Hz, 1H), 3.32- 3.43 (brm, 1H), 3.60 (t, J = 9.15 Hz, 1H), 3.72-3.88 (brm, 1H), 4.18- 4.32 (brm, 2H), 6.89-6.96 (m, 2H), 7.15-7.21 (m, 1H), 8.66 (s, 1H) | 507 | 505 | Single isomer (Stereochemistry of Hydroxyl group: Undetermined) (Diastereomer of F-622) |
| F-622 | | (400 MHz, CDCl3) 0.83-0.87 (m, 6H), 1.05-2.37 (m, 13H), 2.12 (s, 3H), 2.25 (s, 3H), 2.37-2.67 (m, 3H), 2.91 (d, J = 11.69 Hz, 1H), 3.22- 3.32 (brm, 1H), 3.62-3.73 (brm, 1H), 4.16-4.32 (brm, 1H), 4.51- 4.67 (brm, 1H), 6.91-7.00 (m, 2H), 7.32 (d, J = 7.94 Hz, 1H) | 507 | 505 | Single isomer (Stereochemistry of Hydroxyl group: Undetermined) (Diastereomer of F-621) |

TABLE 317

| | | | MS | | Information of |
|---|---|---|---|---|---|
| Example | Chemical structure | NMR | M + H | M − H | structure |
| F-623 | | (400 MHz, DMSO-D6) 0.85 (d, J = 6.62 Hz, 6H), 1.06-1.29 (m, 4H), 1.31 (t, J = 6.95 Hz, 2H), 1.45- 1.58 (m, 1H), 1.85-2.06 (m, 4H), 1.97 (s, 3H), 2.12 (d, J = 1.98 Hz, 3H), 2.39-2.61 (m, 3H), 2.92- 3.08 (m, 2H), 3.20-3.29 (m, 1H), 3.39-3.54 (m, 2H), 3.71-3.91 (m, 2H), 6.93 (t, J = 8.71 Hz, 1H), 6.99 (dd, J = 8.71, 5.62 Hz, 1H), 9.63 (brs, 1H) | 457 | 455 | |
| F-624 | | (400 MHz, CDCl3) 1.01-1.31 (m, 4H), 1.56-1.66 (m, 1H), 1.73- 1.85 (m, 2H), 1.87-2.00 (m, 2H), 2.09 (s, 3H), 2.11-2.23 (m, 1H), 2.27-2.37 (m, 2H), 2.93-3.08 (m, 2H), 3.15-3.26 (m, 1H), 3.33- 3.53 (m, 9H), 3.79-3.92 (m, 2H), 7.13-7.20 (m, 1H), 7.26-7.34 (m, 2H), 9.53 (brs, 1H) | 507 | 505 | |
| F-625 | | (400 MHz, CDCl3) 0.80-0.92 (m, 1H), 0.96-1.07 (m, 15H), 1.32- 1.46 (m, 2H), 1.60-1.77 (m, 3H), 1.81-1.93 (m, 1H), 2.19 (s, 3H), 2.24 (s, 3H), 2.75 (s, 2H), 3.00 (dd, J = 15.16, 4.44 Hz, 1H), 3.38-3.47 (m, 1H), 3.92-4.08 (m, 1H), 4.08- 4.22 (m, 1H), 6.60 (s, 1H), 6.92 (d, J = 8.04 Hz, 1H), 6.94 (s, 1H), 7.36 (d, J = 8.28 Hz, 1H), 8.26 (s, 1H) | 478 | 476 | |

TABLE 318

| | | | MS | | Information of |
|---|---|---|---|---|---|
| Example | Chemical structure | NMR | M + H | M − H | structure |
| F-626 | | (400 MHz, CDCl3) 0.78-0.96 (m, 1H), 0.97-1.10 (m, 15H), 1.36- 1.50 (m, 2H), 1.61-1.82 (m, 3H), 1.82-1.96 (m, 1H), 2.27 (s, 3H), 2.76 (s, 2H), 2.99-3.12 (m, 1H), 3.41-3.51 (m, 1H), 4.12-4.27 (m, 2H), 6.60 (s, 1H), 7.07 (dd, J = 8.72, 2.48 Hz, 1H), 7.12 (d, J = 2.20 Hz, 1H), 7.50 (d, J = 8.72 Hz, 1H), 8.64 (s, 1H) | 498 | 496 | |
| F-627 | | (400 MHz, CDCl3) 0.79-1.08 (m, 13H), 1.31-1.46 (m, 2H), 1.60- 1.80 (m, 6H), 1.81-1.94 (m, 1H), 2.19 (s, 3H), 2.24 (s, 3H), 2.80- 2.92 (m, 2H), 3.01 (dd, J = 14.92, 4.44 Hz, 1H), 3.36-3.49 (m, 1H), 3.96-4.25 (m, 2H), 6.58 (s, 1H), 6.91 (d, J = 8.00, 1H), 6.94 (s, 1H), 7.34 (d, J = 8.00 Hz, 1H), 8.32 (s, 1H) | 478 | 476 | |
| F-628 | | (400 MHz, CDCl3) 0.80-1.09 (m, 13H), 1.35-1.49 (m, 2H), 1.57- 1.79 (m, 6H), 1.82-1.95 (m, 1H), 2.26 (s, 3H), 2.81-2.92 (m, 2H), 2.98-3.11 (m, 1H), 3.40-3.50 (m, 1H), 4.06-4.22 (m, 2H), 6.58 (s, 1H), 7.07 (dd, J = 8.72, 2.44, 1H), 7.12 (d, J = 2.44 Hz, 1H), 7.49 (d, J = 8.72 Hz, 1H), 8.61 (s, 1H) | 498 | 496 | |

TABLE 319

| | | | MS | | Information |
|---|---|---|---|---|---|
| Example | Chemical structure | NMR | M + H | M − H | of structure |
| F-629 | | (400 MHz, CDCl3) 1.01-1.11 (m, 1H), 1.02 (d, J = 6.72 Hz, 6H), 1.17- 1.28 (m, 1H), 1.39 (d, J = 7.42 Hz, 2H), 2.09-2.16 (m, 1H), 2.23 (s, 3H), 2.26 (s, 3H), 2.76 (d, J = 6.96 Hz, 2H), 3.29-3.38 (m, 1H), 3.50-3.58 (m, 1H), 3.95- 4.06 (m, 1H), 4.06-4.18 (m, 2H), 5.74-5.80 (m, 1H), 6.73 (s, 1H), 6.94-7.00 (m, 2H), 7.45 (d, J = 7.88 Hz, 1H), 8.19 (brs, 1H) | 506 | 504 | |
| F-630 | | (400 MHz, CDCl3) 1.02 (d, J = 6.49 Hz, 6H), 1.03-1.10 (m, 1H), 1.13-1.20 (m, 1H), 1.35- 1.43 (m, 2H), 2.09-2.16 (m, 1H), 2.27 (s, 3H), 2.76 (d, J = 6.96 Hz, 2H), 3.32-3.41 (brm, 1H), 3.48- 3.58 (brm, 1H), 3.86-4.00 (brm, 1H), 4.04-4.17 (m, 2H), 5.69- 5.71 (brm, 1H), 6.69 (s, 1H), 7.10- 7.17 (m, 2H), 7.63 (d, J = 10.00 Hz, 1H), 8.46 (brs, 1H) | 526 | 524 | |
| F-631 | | (400 MHz, CDCl3) 1.01 (s, 9H), 1.20-1.36 (m, 3H), 2.12 (s, 3H), 2.19 (s, 3H), 2.25 (s, 3H), 2.74 (s, 2H), 3.50-4.60 (m, 8H), 6.70 (s, 1H), 6.93-6.95 (m, 2H), 7.49 (d, J = 7.94 Hz, 1H), 9.25 (s, 1H) | 505 | 503 | |
| F-632 | | (400 MHz, CDCl3) 0.96 (s, 6H), 1.21-1.32 (m, 3H), 1.63-1.65 (m, 2H), 2.12 (s, 3H), 2.18 (s, 3H), 2.25 (s, 3H), 2.84 (t, J = 7.50 Hz, 2H), 3.52-3.72 (m, 3H), 3.95- 4.55 (m, 6H), 6.69 (s, 1H), 6.93- 6.95 (m, 2H), 7.50 (d, J = 7.72 Hz, 1H), 9.21 (s, 1H) | 505 | 503 | |

TABLE 320

| | | | MS | | Information of |
|---|---|---|---|---|---|
| Example | Chemical structure | NMR | M + H | M − H | structure |
| F-633 | | (400 MHz, CDCl3) 1.01 (d, J = 6.18 Hz, 6H), 1.07-1.14 (m, 1H), 1.19-1.28 (m, 1H), 1.38- 1.47 (m, 2H), 2.09-2.16 (m, 1H), 2.20 (s, 3H), 2.76 (d, J = 6.84 Hz, 2H), 3.35-3.44 (brm, 1H), 3.52- 3.63 (brm, 1H), 4.07-4.19 (brm, 3H), 5.74-5.82 (brm, 1H), 6.74 (brs, 1H), 6.89-7.00 (m, 1H), 7.23- 7.32 (m, 1H), 8.73 (brs, 1H) | 528 | 526 | |
| F-634 | | (400 MHz, CDCl3) 1.02 (d, J = 6.72 Hz, 6H), 1.08-1.16 (m, 1H), 1.24-1.31 (m, 1H), 1.40- 1.47 (m, 2H), 2.09-2.18 (m, 1H), 2.14 (s, 3H), 2.16 (d, J = 1.86 Hz, 3H), 2.77 (d, J = 7.19 Hz, 2H), 3.36- 3.43 (m, 1H), 3.54-3.61 (m, 1H), 4.09-4.15 (m, 3H), 5.82 (dd, J = 10.20, 4.64 Hz, 1H), 6.77 (s, 1H), 6.83 (t, J = 8.81 Hz, 1H), 7.20 (dd, J = 8.70, 5.68 Hz, 1H), 8.49 (brs, 1H) | 524 | 522 | |
| F-635 | | (400 MHz, CDCl3) 0.51-0.59 (m, 1H), 0.74 -0.82 (m, 1H), 0.95- 1.02 (m, 2H), 0.98 (d, J = 5.29 Hz, 6H), 1.00 (d, J = 6.84 Hz, 6H), 1.55- 1.77 (m, 4H), 2.04-2.16 (m, 1H), 2.08 (s, 3H), 2.25 (s, 3H), 2.71 (d, J = 7.06 Hz, 2H), 2.79 (dd, J = 13.89, 4.85 Hz, 1H), 2.93 (dd, J = 13.89, 10.14 Hz, 1H), 3.69-3.79 (m, 1H), 6.39 (s, 1H), 6.94 (s, 1H), 6.95 (d, J = 7.94 Hz, 1H), 7.18 (brs, 1H), 7.49 (d, J = 7.94 Hz, 1H) | 464 | 462 | |

TABLE 321

| | | | MS | | Information of |
|---|---|---|---|---|---|
| Example | Chemical structure | NMR | M + H | M − H | structure |
| F-636 | | (400 MHz, CDCl3) 0.32-0.52 (m, 4H), 0.82-0.92 (m, 6H), 1.32- 1.45 (m, 2H), 1.49-1.61 (m, 1H), 1.80-2.04 (m, 3H), 2.04-2.14 (m, 3H), 2.18-2.30 (m, 3H), 2.31- 2.42 (m, 4H), 2.41-2.49 (m, 2H), 2.72-2.81 (m, 1H), 2.90-3.01 (m, 1H), 3.25 (s, 3H), 3.44-3.57 (m, 1H), 3.57-3.68 (m, 1H), 6.92- 7.01 (m, 2H), 7.41-7.49 (m, 1H), 7.67 (brs, 1H) | 544 | 542 | |
| F-637 | | (400 MHz, DMSO-D6) 0.42-0.55 (m, 2H), 0.73-0.91 (m, 2H), 0.85 (d, J = 6.80 Hz, 6H), 1.26-1.35 (m, 2H), 1.39-1.57 (m, 2H), 1.74- 1.88 (m, 2H), 2.06 (s, 3H), 2.27- 2.44 (m, 3H), 2.59-2.73 (m, 2H), 2.73-2.85 (m, 2H), 3.14 (s, 3H), 3.48-3.60 (m, 1H), 3.77-3.87 (m, 1H), 6.96 (dt, J = 8.80, 2.80 Hz, 1H), 7.04 (dd, J = 9.60, 2.80 Hz, 1H), 7.26 (dd, J = 8.80, 5.60 Hz, 1H), 9.34 (s, 1H), 11.79 (s, 1H) | 534 | 532 | |
| F-638 | | (400 MHz, CDCl3) 0.45-0.51 (m, 2H), 0.88 (s, 9H), 1.36-1.43 (m, 3H), 1.55-1.60 (m, 1H), 1.98- 2.02 (m, 2H), 2.12 (s, 3H), 2.28 (s, 3H), 2.36-2.38 (m, 2H), 2.87- 2.92 (m, 3H), 3.11 (dd, J = 10.32, 5.16 Hz, 1H), 3.53 (s, 1H), 3.93 (s, 1H), 6.99 (s, 2H), 7.19 (d, J = 6.03 Hz, 1H), 7.48 (d, J = 8.58 Hz, 1H) | 434 | 432 | |

TABLE 322

| | | | MS | | Information of |
|---|---|---|---|---|---|
| Example | Chemical structure | NMR | M + H | M − H | structure |
| F-639 | | (400 MHz, CDCl3) 0.47 (d, J = 5.33 Hz, 2H), 0.88-0.89 (m, 8H), 1.34-1.41 (m, 12H), 1.52- 1.59 (m, 1H), 1.97 (d, J = 9.74 Hz, 2H), 2.16 (s, 3H), 2.28 (s, 3H), 2.38 (t, J = 6.84 Hz, 3H), 2.57-2.65 (m, 2H), 2.85-2.92 (m, 2H), 3.50- 3.52 (m, 1H), 3.86-3.87 (m, 1H), 5.92 (s, 1H), 6.98 (q, J = 6.03 Hz, 2H), 7.59 (d, J = 8.58 Hz, 1H), 8.03 (s, 1H) | 508 | 506 | |
| F-640 | | (400 MHz, CDCl3) 0.46-0.47 (m, 2H), 0.87-0.94 (m, 8H), 1.37 (dd, J = 13.45, 6.40 Hz, 3H), 1.52-1.59 (m, 1H), 1.96-1.99 (m, 2H), 2.18 (s, 3H), 2.28 (s, 3H), 2.37-2.40 (m, 3H), 2.90-2.93 (m, 3H), 3.21- 3.25 (m, 1H), 3.53 (t, J = 8.82 Hz, 1H), 3.82 (t, J = 6.40 Hz, 1H), 6.99- 7.00 (m, 2H), 7.51 (d, J = 8.82 Hz, 1H), 7.97 (s, 1H) | 493 | 491 | |
| F-641 | | (400 MHz, CDCl3) 0.46 (s, 2H), 0.87-0.94 (m, 8H), 1.34-1.44 (m, 3H), 1.52-1.59 (m, 1H), 1.94- 1.99 (m, 2H), 2.19 (s, 3H), 2.28 (s, 3H), 2.40 (s, 3H), 2.79 (dd, J = 14.11, 7.06 Hz, 1H), 2.94 (dd, J = 14.22, 4.96 Hz, 1H), 3.25 (dd, J = 14.67, 8.71 Hz, 1H), 3.53 (t, J = 8.82 Hz, 1H), 3.74 (dd, J = 14.78, 4.85 Hz, 1H), 3.84 (s, 1H), 6.98- 7.00 (m, 2H), 7.53 (d, J = 8.38 Hz, | 477 | 475 | |
| | | 1H), 8.36 (s, 1H) | | | |

TABLE 323

| | | | MS | | Information of |
|---|---|---|---|---|---|
| Example | Chemical structure | NMR | M + H | M − H | structure |
| F-642 | | (400 MHz, CDCl3) 0.48 (s, 2H), 0.87-0.88 (m, 8H), 1.34-1.42 (m, 3H), 1.57 (d, J = 6.84 Hz, 1H), 1.96 (t, J = 10.26 Hz, 2H), 2.15 (s, 3H), 2.27 (s, 3H), 2.33-2.40 (m, 3H), 2.72-2.80 (m, 2H), 2.92 (d, J = 7.28 Hz, 2H), 3.49-3.51 (m, 1H), 3.89 (t, J = 6.73 Hz, 1H), 5.45 (s, 1H), 6.28 (s, 1H), 6.97 (s, 2H), 7.57 (d, J = 8.60 Hz, 1H), 7.87 (s, 1H) | 452 | 450 | |
| F-643 | | (400 MHz, DMSO-D6) 0.33-0.44 (m, 1H), 0.44-0.53 (m, 1H), 0.74- 0.83 (m, 1H), 0.83-0.88 (m, 7H), 1.27-1.34 (m, 2H), 1.36-1.47 (m, 1H), 1.47-1.57 (m, 1H), 1.76- 1.91 (m, 2H), 1.97-2.06 (m, 6H), 2.22 (s, 4H), 2.29-2.42 (m, 4H), 2.65-2.83 (m, 2H), 3.41-3.60 (m, 1H), 6.89-6.94 (m, 1H), 6.97 (br s, 1H), 7.12 (d, J = 8.40 Hz, 1H), 9.24 (brs, 1H), 12.11 (brs, 1H) | 507 | 505 | |
| F-644 | | (400 MHz, DMSO-D6) 0.25-0.43 (m, 1H), 0.43-0.51 (m, 1H), 0.70- 0.83 (m, 2H), 0.83-0.89 (m, 7H), 1.26-1.35 (m, 2H), 1.35-1.45 (m, 1H), 1.47-1.57 (m, 1H), 1.75- 1.94 (m, 2H), 1.97-2.02 (m, 4H), 2.10-2.20 (m, 2H), 2.22 (s, 3H), 2.31-2.44 (m, 2H), 2.69-2.91 (m, 4H), 3.40-3.60 (m, 1H), 6.89- 6.94 (m, 1H), 6.97 (brs, 1H), 7.11 (d, J = 8.40 Hz, 1H), 9.26 (brs, 1H) | 491 | 489 | |

TABLE 324

| | | | MS | | Information of |
|---|---|---|---|---|---|
| Example | Chemical structure | NMR | M + H | M − H | structure |
| F-645 | | (400 MHz, CDCl3) 0.88 (d, J = 6.72 Hz, 6H), 1.32-1.39 (m, 5H), 1.51-1.58 (m, 1H), 1.98- 2.09 (m, 2H), 2.21 (s, 3H), 2.29 (s, 3H), 2.40-2.54 (m, 3H), 2.94 (t, J = 6.96 Hz, 2H), 3.18 (t, J = 6.84 Hz, 2H), 3.58 (dd, J = 17.04, 8.93 Hz, 1H), 4.25 (q, J = 7.11 Hz, 2H), 6.13 (d, J = 16.46 Hz, 1H), 6.98-7.03 (m, 2H), 7.36 (brs, 1H), 7.44 (d, | 453 | 451 | |
| | | J = 16.23 Hz, 1H), 7.64 (d, | | | |
| | | J = 8.12 Hz, 1H) | | | |
| F-646 | | (400 MHz, DMSO-D6) 0.83-0.86 (m, 1H), 0.84 (d, J = 6.49 Hz, 6H), 1.31 (t, J = 6.84 Hz, 2H), 1.44-1.55 (m, 1H), 1.87 (dd, J = 16.46, 7.42 Hz, 2H), 2.11 (s, 3H), 2.23 (s, 3H), 2.36-2.53 (m, 2H), 2.75 (t, J = 7.30 Hz, 2H), 3.02 (t, J = 7.19 Hz, 2H), 3.69-3.79 (m, 1H), 6.10 (d, J = 16.23 Hz, 1H), 6.93 (d, J = 7.88 Hz, 1H), 6.99 (s, 1H), 7.21 | 425 | 423 | |
| | | (d, J = 7.88 Hz, 1H), 7.36 (d, | | | |
| | | J = 16.23 Hz, 1H), 9.27 (s, 1H), | | | |
| | | 12.48 (brs, 1H) | | | |

TABLE 325

| | | | MS | | Information of |
|---|---|---|---|---|---|
| Example | Chemical structure | NMR | M + H | M − H | structure |
| F-647 | | (400 MHz, DMSO-D6) 0.37-0.42 (m, 1H), 0.48-0.53 (m, 1H), 0.76- 0.81 (m, 2H), 0.84 (d, J = 6.96 Hz, 6H), 1.27-1.32 (m, 2H), 1.39- 1.46 (m, 1H), 1.48-1.56 (m, 1H), 1.76-1.89 (m, 2H), 1.96 (s, 3H), 2.21 (s, 3H), 2.31-2.39 (m, 3H), 2.64-2.71 (m, 1H), 2.77 (dd, J = 14.73, 9.62 Hz, 1H), 3.29-3.46 (m, 2H), 3.49-3.60 (m, 2H), 3.62 (s, 2H), 6.90 (d, J = 8.12 Hz, 1H), 6.95 (s, 1H), 7.07 (d, J = 8.58 Hz, | 491 | 489 | |
| | | 1H), 8.38 (t, J = 5.80 Hz, 1H), 9.24 | | | |
| | | (s, 1H) | | | |
| F-648 | | (400 MHz, DMSO-D6) 0.86 (s, 9H), 1.04-1.24 (m, 4H), 1.36 (d, J = 6.26 Hz, 2H), 2.01-2.16 (m, 2H), 2.10 (s, 1.5H), 2.11 (s, 1.5H), 2.23 (s, 3H), 2.43-2.61 (m, 4H), 2.83 (s, 3H), 2.84 (s, 3H), 3.21- 3.31 (m, 1H), 3.55-4.32 (m, 9H), 6.95 (d, J = 8.58 Hz, 1H), 7.01 (s, 1H), 7.15-7.21 (m, 1H), 9.79 (s, 0.5H), 9.82-9.96 (m, 1H), 9.88 (s, 0.5H) | 535 | 533 | |
| F-649 | | (400 MHz, DMSO-D6) 0.86 (s, 9H), 0.99-1.19 (m, 4H), 1.07 (t, J = 6.96 Hz, 3H), 1.16 (t, J = 6.96 Hz, 3H), 1.36 (d, J = 6.26 Hz, 2H), 2.01- 2.26 (m, 2H), 2.09 (s, 3H), 2.23 (s, 3H), 2.42-2.60 (m, 3H), 3.11-3.19 (m, 1H), 3.25 (q, J = 6.96 Hz, 2H), 3.30-3.40 (m, 2H), 3.36-4.46 (m, 8H), 4.56 (s, 2H), 6.95 (d, J = 8.12 Hz, 1H), 7.01 (s, 1H), 7.13- 7.26 (m, 1H), 9.89 (s, 0.5H), 9.95 (s, 0.5H), 10.53-10.73 (m, 1H) | 563 | 561 | |

TABLE 326

| | | | MS | | Information of |
|---|---|---|---|---|---|
| Example | Chemical structure | NMR | M + H | M − H | structure |
| F-650 | | (400 MHz, DMSO-D6) 0.86 (s, 9H), 1.03-1.28 (m, 10H), 1.36 (d, J = 6.26 Hz, 2H), 2.03-2.20 (m, 2H), 2.10 (s, 1.5H), 2.12 (s, 1.5H), 2.23 (s, 3H), 2.43-2.75 (m, 4H), 3.09-3.24 (m, 4H), 3.24-3.36 (m, 1H), 3.57-4.32 (m, 9H), 6.95 (d, J = 8.12 Hz, 1H), 7.01 (s, 1H), 7.12- 7.22 (m, 1H), 9.39-9.58 (m, 1H), 9.84 (s, 0.5H), 9.93 (s, 0.5H) | 563 | 561 | |
| F-651 | | (400 MHz, DMSO-D6) 0.86 (s, 9H), 0.99-1.27 (m, 4H), 1.35 (d, J = 6.26 Hz, 2H), 1.99-2.16 (m, 2H), 2.08 (s, 3H), 2.22 (s, 3H), 2.42-2.60 (m, 4H), 2.75 (d, J = 4.17 Hz, 3H), 2.87-3.09 (m, 2H), 3.13-3.30 (m, 3H), 3.31- 3.41 (m, 2H), 3.54-3.69 (m, 2H), 3.69-3.85 (m, 4H), 3.86-3.98 (m, 2H), 4.03-4.13 (m, 1H), 6.93 (d, J = 8.12 Hz, 1H), 7.00 (s, 1H), 7.18 | 576 | 574 | |
| | | (d, J = 8.12 Hz, 1H), 9.67 (s, 1H), | | | |
| | | 10.94-11.11 (m, 1H) | | | |
| F-652 | | (400 MHz, DMSO-D6) 0.86 (s, 9H), 0.98-1.21 (m, 4H), 1.35 (d, J = 6.26 Hz, 2H), 2.01-2.16 (m, 2H), 2.10 (s, 3H), 2.23 (s, 3H), 2.43-2.61 (m, 4H), 2.90 (s, 3H), 2.94 (s, 3H), 3.12-3.20 (m, 1H), 3.43-4.46 (m, 7H), 4.54 (s, 2H), 6.95 (d, J = 8.12 Hz, 1H), 7.01 (s, 1H), 7.15-7.25 (m, 1H), 9.84- 10.02 (m, 1H), 10.51-10.84 (m, 1H) | 535 | 533 | |

TABLE 327

| | | | MS | | Information of |
|---|---|---|---|---|---|
| Example | Chemical structure | NMR | M + H | M − H | structure |
| F-653 | | (400 MHz, DMSO-D6) 0.85 (d, J = 6.49 Hz, 6H), 0.97-1.19 (m, 4H), 1.07 (t, J = 6.96 Hz, 3H), 1.16 (t, J = 6.96 Hz, 3H), 1.32 (t, J = 6.96 Hz, 2H), 1.40-1.59 (m, 1H), 1.99-2.15 (m, 2H), 2.10 (s, 3H), 2.23 (s, 3H), 2.36-2.59 (m, 3H), 3.11-3.21 (m, 1H), 3.26 (q, J = 7.19 Hz, 2H), 3.30-3.42 (m, 2H), 3.46-4.46 (m, 8H), 4.57 (s, 2H), 6.96 (d, J = 8.12 Hz, 1H), 7.01 (s, 1H), 7.13-7.26 (m, 1H), 9.89 (s, 0.5H), 9.95 (s, 0.5H), 10.53- 10.76 (m, 1H) | 549 | 547 | |
| F-654 | | (400 MHz, DMSO-D6) 0.85 (d, J = 6.26 Hz, 6H), 1.03-1.28 (m, 10H), 1.32 (t, J = 6.96 Hz, 2H), 1.41- 1.59 (m, 1H), 2.00-2.17 (m, 2H), 2.10 (s, 1.5H), 2.11 (s, 1.5H), 2.23 (s, 3H), 2.36-2.63 (m, 4H), 3.07- 3.35 (m, 5H), 3.55- 4.30 (m, 8H), 6.95 (d, J = 8.12 Hz, 1H), 7.01 (s, 1H), 7.15-7.22 (m, 1H), 9.35- 9.55 (m, 1H), 9.81 (s, 0.5H), 9.90 (s, 0.5H) | 549 | 547 | |

TABLE 328

| | | | MS | | Information of |
|---|---|---|---|---|---|
| Example | Chemical structure | NMR | M + H | M − H | structure |
| F-655 | | (400 MHz, DMSO-D6) 0.85 (d, J = 6.26 Hz, 6H), 0.99-1.25 (m, 4H), 1.31 (t, J = 7.19 Hz, 2H), 1.41- 1.59 (m, 1H), 1.94-2.16 (m, 2H), 2.07 (s, 3H), 2.22 (s, 3H), 2.35- 2.59 (m, 4H), 2.75 (d, J = 4.64 Hz, 3H), 2.88-3.08 (m, 2H), 3.11- 3.30 (m, 3H), 3.31-3.40 (m, 2H), 3.54-3.69 (m, 2H), 3.69-3.85 (m, 4H), 3.86-3.95 (m, 2H), 4.03- | 562 | 560 | |
| | | 4.12 (m, 1H), 6.94 (d, J = 7.88 Hz, | | | |
| | | 1H), 7.00 (s, 1H), 7.18 (d, | | | |
| | | J = 7.88 Hz, 1H), 9.65 (s, 1H), | | | |
| | | 10.77-11.00 (m, 1H) | | | |
| F-656 | | (400 MHz, DMSO-D6) 0.85 (d, J = 6.26 Hz, 6H), 0.97-1.20 (m, 4H), 1.32 (t, J = 6.96 Hz, 2H), 1.40- 1.58 (m, 1H), 1.97-2.15 (m, 2H), 2.09 (s, 3H), 2.23 (s, 3H), 2.36- 2.59 (m, 4H), 2.90 (s, 3H), 2.94 (s, 3H), 3.10-3.21 (m, 1H), 3.45- 4.46 (m, 7H), 4.53 (s, 2H), 6.95 (d, J = 8.15 Hz, 1H), 7.01 (s, 1H), 7.15- 7.25 (m, 1H), 9.82-10.00 (m, 1H), 10.52 -10.91 (m, 1H) | 521 | 519 | |
| F-657 | | (400 MHz, DMSO-D6) 0.85 (d, J = 6.49 Hz, 6H), 1.02-1.28 (m, 4H), 1.32 (d, J = 6.96 Hz, 2H), 1.40- 1.60 (m, 1H), 1.96-2.17 (m, 2H), 2.10 (s, 1.5H), 2.11 (s, 1.5H), 2.23 (s, 3H), 2.37-2.59 (m, 4H), 2.84 (brs, 6H), 3.19-3.31 (m, 1H), 3.56- 4.34 (m, 9H), 6.95 (d, J = 8.12 Hz, 1H), 7.01 (s, 1H), 7.14-7.23 (m, 1H), 9.79 (s, 0.5H), 9.82-9.99 (m, 1H), 9.88 (s, 0.5H) | 521 | 519 | |

TABLE 329

| | | | MS | | Information of |
|---|---|---|---|---|---|
| Example | Chemical structure | NMR | M + H | M − H | structure |
| F-658 | | (400 MHz, DMSO-D6) 0.85 (d, J = 6.26 Hz, 6H), 0.99-1.25 (m, 4H), 1.26-1.36 (m, 7H), 1.40- 1.60 (m, 1H), 1.92-2.18 (m, 2H), 2.08 (s, 1.5H), 2.09 (s, 1.5H), 2.23 (s, 3H), 2.37-2.59 (m, 4H), 3.16- 3.29 (m, 1H), 3.45-4.20 (m, 7H), 4.44 - 4.59 (m, 2H), 6.94 (d, J = 8.35 Hz, 1H), 7.01 (s, 1H), 7.13- 7.23 (m, 1H), 9.63 (s, 0.5H), 9.65 (s, 0.5H) | 522 | 520 | |
| F-659 | | (400 MHz, DMSO-D6) 0.74-0.76 (brm, 1H), 0.84-0.87 (brm, 1H), 0.91-0.97 (m, 8H), 1.05-1.08 (br m, 2H), 1.23 (brs, 2H), 1.99 (s, 3H), 2.20 (s, 3H), 2.74-2.76 (br m, 1H), 3.36 (brs, 3H), 3.47 (brs, 2H), 3.89 (s, 12H), 6.73-6.75 (br m, 1H), 6.89 (d, J = 8.58 Hz, 1H), 6.94- 6.96 (brm, 1H), 7.09 (d, J = 7.19 Hz, 1H), 9.32 (brs, 1H) | 452 | 450 | |
| F-660 | | (400 MHz, DMSO-D6) 0.69-0.80 (m, 1H), 0.84-0.87 (m, 2H), 0.94 (d, J = 6.72 Hz, 8H), 1.06-1.09 (m, 4H), 1.23-1.26 (brm, 3H), 1.90 (s, 3H), 2.05 (s, 6H), 2.63-2.70 (m, 2H), 2.74-2.76 (m, 2H), 2.93- 3.07 (m, 1H), 3.35-3.38 (m, 2H), 3.82-3.91 (m, 2H), 6.74 (s, 1H), 7.09-7.19 (m, 1H), 7.23-7.26 (m, 1H), 7.28-7.30 (m, 1H), 7.30- 7.33 (m, 1H), 9.46 (s, 1H) | 472 | 470 | |

TABLE 330

| | | | MS | | Information |
|---|---|---|---|---|---|
| Example | Chemical structure | NMR | M + H | M − H | of structure |
| F-661 | | (400 MHz, DMSO-D6) 0.75-0.77 (brm, 1H), 0.84-0.87 (brm, 3H), 0.94-0.95 (m, 6H), 1.06-1.08 (brm, 2H), 1.23-1.25 (brm, 3H), 1.91 (s, 3H), 2.09 (s, 3H), 2.75 (d, J = 6.26 Hz, 2H), 2.80-2.92 (brm, 2H), 2.98-3.00 (brm, 2H), 3.35- 3.38 (brm, 3H), 3.46-3.49 (brm, 2H), 3.85-3.88 (brm, 2H), 6.74- 6.76 (m, 1H), 6.91-6.95 (m, 2H), 9.51 (brs, 1H) | 470 | 468 | |
| F-662 | | (400 MHz, DMSO-D6) 0.76-0.78 (brm, 2H), 0.84-0.87 (brm, 2H), 0.94-0.95 (m, 6H), 1.06-1.09 (brm, 4H), 1.23-1.25 (brm, 3H), 1.79-1.99 (m, 6H), 2.05 (s, 2H), 2.69-2.78 (m, 3H), 2.82-2.94 (m, 2H), 2.96-3.07 (m, 2H), 3.20- 3.42 (m, 3H), 3.42-3.57 (m, 2H), 3.82-3.94 (m, 3H), 6.75 (s, 1H), 7.04-7.07 (m, 1H), 7.16-7.19 (m, 2H), 9.64 (s, 1H) | 474 | 472 | |
| F-663 | | (400 MHz, CDCl3) 0.86-0.91 (m, 1H), 0.87 (d, J = 6.72 Hz, 6H), 1.20- 1.29 (m, 3H), 1.36 (t, J = 6.72 Hz, 2H), 1.52-1.58 (m, 1H), 1.95- 2.02 (m, 2H), 2.17 (s, 3H), 2.28 (s, 3H), 2.35-2.45 (m, 4H), 2.63 (t, J = 7.65 Hz, 2H), 2.91 (t, J = 6.61 Hz, 2H), 3.01 (t, J = 6.72 Hz, 2H), 3.37- 3.46 (m, 1H), 4.09-4.14 (m, 2H), 6.97-7.02 (m, 2H), 7.44-7.51 (m, | 455 | 453 | |
| | | 1H), 7.63 (d, J = 7.65 Hz, 1H) | | | |

TABLE 331

| | | | MS | | Information |
|---|---|---|---|---|---|
| Example | Chemical structure | NMR | M + H | M − H | of structure |
| F-664 | | (400 MHz, CDCl3) 0.86-0.91 (m, 1H), 0.87 (d, J = 6.72 Hz, 6H), 1.35 (t, J = 6.84 Hz, 2H), 1.51-1.58 (m, 1H), 1.93-2.04 (m, 2H), 2.14 (s, 3H), 2.28 (s, 3H), 2.33-2.41 (m, 2H), 2.48 (t, J = 7.19 Hz, 2H), 2.64 (t, J = 7.42 Hz, 2H), 2.91 (t, J = 6.38 Hz, 2H), 3.01 (t, J = 6.38 Hz, 2H), 3.35-3.46 (m, 1H), 6.96- 7.01 (m, 2H), 7.49 (brs, 1H), 7.53 (d, J = 8.58 Hz, 1H) | 427 | 425 | |
| F-665 | | (400 MHz, CDCl3) 0.42 (d, J = 4.19 Hz, 2H), 0.82-0.89 (m, 8H), 1.34-1.35 (m, 3H), 1.51- 1.58 (m, 1H), 1.98 (t, J = 8.82 Hz, 2H), 2.15 (s, 3H), 2.27 (s, 3H), 2.35-2.38 (m, 3H), 2.89 (t, J = 6.95 Hz, 2H), 3.07 (t, J = 6.95 Hz, 2H), 3.48-3.50 (m, 1H), 6.97- | 395 | 393 | |
| | | 6.99 (m, 2H), 7.53 (s, 1H), 7.63 (d, | | | |
| | | J = 7.94 Hz, 1H) | | | |
| F-666 | | (400 MHz, CDCl3) 0.36-0.42 (m, 2H), 0.80-0.89 (m, 8H), 1.28- 1.36 (m, 3H), 1.51-1.57 (m, 1H), 1.95 (t, J = 9.92 Hz, 2H), 2.05 (s, 3H), 2.21 (s, 3H), 2.39 (brs, 3H), 2.72 (dd, J = 16.76, 4.19 Hz, 1H), 2.86-2.95 (m, 2H), 3.12 (dd, J = 16.10, 9.70 Hz, 1H), 3.46-3.53 (m, 2H), 6.92-6.93 (m, 2H), 7.48 | 453 | 451 | |
| | | d, J = 8.60 Hz, 1H), 8.07 (s, 1H) | | | |

TABLE 332

| | | | MS | | Information |
|---|---|---|---|---|---|
| Example | Chemical structure | NMR | M + H | M − H | of structure |
| F-667 | | (400 Mz, DMSO-D6) 0.47 (td, J = 5.62, 3.86 Hz, 2H), 0.78-0.85 (m, 8H), 1.26 (dd, J = 16.23, 9.28 Hz, 2H), 1.40-1.53 (m, 2H), 1.76-1.84 (m, 2H), 2.29-2.37 (m, 3H), 2.37 (s, 3H), 2.54-2.81 (m, 4H), 3.47-3.54 (1H, m), 3.74-3.82 (m, 1H), 7.16 (d, J = 8.35 Hz, 1H), | 440 | 438 | |
| | | 7.85 (dd, J = 8.46, 2.67 Hz, 1H), | | | |
| | | 8.53 (d, J = 2.32 Hz, 1H), 10.07 (s, | | | |
| | | 1H), 12.16 (brs, 1H) | | | |
| F-668 | | (400 MHz, CDCl3) 0.36-0.42 (m, 2H), 0.81-0.88 (m, 8H), 1.28- 1.36 (m, 3H), 1.51-1.57 (m, 1H), 1.93-2.06 (m, 5H), 2.24 (s, 3H), 2.36 (t, J = 16.54 Hz, 3H), 2.71 (dd, J = 16.87, 4.30 Hz, 1H), 2.85-2.96 (m, 2H), 3.12 (dd, J = 15.99, 9.59 Hz, 1H), 3.46-3.52 (m, 2H), 6.90-6.92 (m, 2H), 7.46 (d, | 453 | 451 | |
| | | J = 8.82 Hz, 1H), 8.07 (s, 1H) | | | |
| F-669 | | (400 Mz, DMSO-D6) 0.44-0.49 (m, 2H), 0.78-0.85 (m, 8H), 1.29 (t, J = 6.84 Hz, 2H), 1.39-1.56 (m, 2H), 1.82 (m, 2H), 2.05 (s, 3H), 2.29-2.37 (m, 3H), 2.54-2.82 (m, 4H), 3.45 (s, 3H), 3.49-3.58 (m, 1H), 3.71-3.78 (m, 1H), 5.89 (s, 1H), 9.89 (s, 1H), 12.24 (brs, 1H) | 443 | 441 | |

TABLE 333

| | | | MS | | Information |
|---|---|---|---|---|---|
| Example | Chemical structure | NMR | M + H | M − H | of structure |
| F-670 | | (400 Mz, DMSO-D6) 0.46-0.47 (m, 2H), 0.78-0.85 (m, 8H), 1.28 (t, J = 6.84 Hz, 2H), 1.40-1.56 (m, 2H), 1.78-1.84 (m, 2H), 2.32- 2.35 (m, 6H), 2.51-2.57 (m, 1H), 2.66-2.70 (m, 2H), 2.81 (dd, J = 15.65, 7.77 Hz, 1H), 3.48-3.54 (m, 1H), 3.74 (dd, J = 14.49, 7.30 Hz, 1H), 6.57 (s, 1H), 10.92 (s, | 430 | 428 | |
| | | 1H), 12.25 (brs, 1H) | | | |
| F-671 | | (400 MHz, CDCl3) 0.31-0.33 (m, 1H), 0.49-0.50 (m, 1H), 0.83- 0.86 (m, 8H), 1.32-1.34 (m, 3H), 1.52 (dd, J = 13.45, 6.62 Hz, 1H), 1.95-1.98 (m, 6H), 2.20-2.24 (m, 4H), 2.36 (s, 3H), 2.49 (s, 1H), 2.59 (s, 1H), 2.74 (dd, J = 15.44, 3.97 Hz, 1H), 3.20 (dd, J = 15.33, 11.36 Hz, 1H), 3.34 (s, 1H), 3.46- 3.49 (m, 1H), 6.89-6.90 (m, 2H), | 467 | 465 | |
| | | 7.32 (d, J = 8.60 Hz, 1H), 8.49 (s, | | | |
| | | 1H) | | | |
| F-672 | | (400 MHz, CDCl3) 0.34-0.52 (m, 2H), 0.77-0.93 (m, 2H), 0.87 (s, 9H), 1.31-1.42 (m, 1H), 1.38 (d, J = 5.84 Hz, 2H), 1.44-1.70 (m, 3H), 1.76-2.13 (m, 4H), 2.27 (s, 3H), 2.30-2.46 (m, 3H), 2.65- 2.79 (m, 1H), 2.91-3.07 (m, 1H), 3.42-3.71 (m, 4H), 7.02 (d, J = 8.40 Hz, 1H), 7.14 (s, 1H), 7.75 (s, 1H), 8.11 (d, J = 8.40 Hz, 1H) | 487 | 485 | |

TABLE 334

| | | | MS | | Information |
|---|---|---|---|---|---|
| Example | Chemical structure | NMR | M + H | M − H | of structure |
| F-673 | | (400 MHz, DMSO-D6) 0.34-0.52 (m, 2H), 0.75-0.87 (m, 2H), 0.84 (s, 9H), 1.32 (d, J = 6.00 Hz, 2H), 1.36-1.45 (m, 1H), 1.77-1.98 (m, 4H), 2.12-2.25 (m, 2H), 2.31- 2.43 (m, 3H), 2.48 (s, 3H), 2.63 (s, 3H), 2.80 (dd, J = 15.20, 6.80 Hz, 1H), 2.88 (dd, J = 15.20, 8.40 Hz, 1H), 3.23-3.68 (m, 2H), 7.60 (d, | 482 | 480 | |
| | | J = 8.00 Hz, 1H), 8.27 (d, J = 8.00 Hz, | | | |
| | | 1H), 10.10 (s, 1H) | | | |
| F-674 | | (400 MHz, DMSO-D6) 0.32-0.53 (m, 2H), 0.73-0.88 (m, 2H), 0.85 (s, 9H), 1.33 (d, J = 6.00 Hz, 2H), 1.36-1.45 (m, 1H), 1.77-1.99 (m, 4H), 2.10-2.23 (m, 2H), 2.27 (s, 3H), 2.31-2.44 (m, 3H), 2.55 (s, 3H), 2.76 (dd, J = 14.80, 6.80 Hz, 1H), 2.83 (dd, J = 14.80, 8.40 Hz, 1H), 3.41-3.59 (m, 2H), 8.58 (s, 1H), 9.82 (s, 1H) | 483 | 481 | |
| F-675 | | (400 MHz, DMSO-D6) 0.34-0.52 (m, 2H), 0.73-0.89 (m, 2H), 0.84 (s, 9H), 1.32 (d, J = 5.60 Hz, 2H), 1.36-1.46 (m, 1H), 1.77-1.98 (m, 4H), 2.11-2.25 (m, 2H), 2.29- 2.45 (m, 3H), 2.32 (s, 3H), 2.59 (s, 3H), 2.82 (dd, J = 15.20, 6.80 Hz, 1H), 2.91 (dd, J = 15.20, 8.40 Hz, 1H), 3.26-3.71 (m, 2H), 7.70 (s, 1H), 8.74 (s, 1H), 9.95 (s, 1H) | 482 | 480 | |

TABLE 335

| | | | MS | | Information |
|---|---|---|---|---|---|
| Example | Chemical structure | NMR | M + H | M − H | of structure |
| F-676 | | (400 MHz, DMSO-D6) 0.83 (s, 9H), 1.31 (d, J = 6.75 Hz, 2H), 1.75- 1.80 (m, 3H), 1.90 (t, J = 5.35 Hz, 1H), 2.14 (t, J = 7.56 Hz, 2H), 2.27 (d, J = 13.49 Hz, 3H), 2.34-2.43 (m, 3H), 2.68 (t, J = 5.70 Hz, 2H), 3.24- 3.28 (m, 2H), 3.38-3.47 (m, 1H), 6.21 (s, 1H), 7.08 (d, J = 8.14 Hz, 1H), 7.42 (d, J = 8.14 Hz, 1H), 9.41 (s, 1H), 12.06 (s, 1H) | 461 | 459 | |
| F-677 | | (400 MHz, CDCl3) 0.36-0.39 (m, 2H), 0.80-0.84 (m, 2H), 0.87 (s, 9H), 1.28-1.35 (m, 1H), 1.38 (d, J = 5.95 Hz, 2H), 1.96-2.06 (m, 2H), 2.28 (s, 3H), 2.34-2.44 (m, 3H), 2.76 (dd, J = 17.09, 4.08 Hz, 1H), 2.89 (dd, J = 15.99, 5.84 Hz, 1H), 2.97 (dd, J = 16.98, 9.04 Hz, 1H), 3.11 (dd, J = 16.10, 8.82 Hz, | 487 | 485 | |
| | | 1H), 3.45-3.58 (m, 2H), 7.02 (dd, | | | |
| | | J = 8.38, 1.54 Hz, 1H), 7.14 (d, | | | |
| | | J = 1.32 Hz, 1H), 8.08 (d, J = 8.38 Hz, | | | |
| | | 1H), 8.31 (s, 1H) | | | |
| F-678 | | (400 MHz, CDCl3) 0.36-0.39 (m, 2H), 0.80-0.84 (m, 2H), 0.87 (s, 9H), 1.28-1.35 (m, 1H), 1.39 (d, J = 5.95 Hz, 2H), 1.99-2.05 (m, 2H), 2.28 (s, 3H), 2.34-2.43 (m, 3H), 2.79 (dd, J = 16.76, 3.97 Hz, 1H), 2.90 (dd, J = 15.55, 5.18 Hz, 1H), 2.96 (dd, J = 16.87, 8.71 Hz, 1H), 3.11 (dd, J = 15.99, 8.93 Hz, | 487 | 485 | |
| | | 1H), 3.45-3.59 (m, 2H), 7.03 (d, | | | |
| | | J = 6.84 Hz, 1H), 7.15 (s, 1H), 8.09 | | | |
| | | (d, J = 8.38 Hz, 1H), 8.36 (s, 1H) | | | |

TABLE 336

| | | | MS | | Information |
|---|---|---|---|---|---|
| Example | Chemical structure | NMR | M + H | M − H | of structure |
| F-679 | | (400 MHz, DMSO-D6) 0.32-0.43 (m, 1H), 0.45-0.56 (m, 1H), 0.74- 0.95 (m, 2H), 0.86 (s, 9H), 1.29- 1.46 (m, 1H), 1.34 (d, J = 6.00 Hz, 2H), 1.65 (s, 3H), 1.77-1.97 (m, 4H), 2.06-2.24 (m, 2H), 2.11 (s, 3H), 2.31-2.46 (m, 3H), 2.64- 2.85 (m, 2H), 3.21-3.61 (m, 2H), 10.50 (s, 1H), 12.14 (brs, 1H) | 472 | 470 | |
| F-680 | | (400 MHz, DMSO-D6) 0.36-0.45 (m, 1H), 0.45-0.55 (m, 1H), 0.74- 0.90 (m, 2H), 0.85 (s, 9H), 1.33 (d, J = 6.00 Hz, 2H), 1.37-1.48 (m, 1H), 1.77-1.95 (m, 4H), 2.09- 2.25 (m, 2H), 2.19 (s, 3H), 2.30- 2.46 (m, 3H), 2.37 (s, 3H), 2.75 (dd, J = 15.60, 6.80 Hz, 1H), 2.83 (dd, J = 15.60, 7.60 Hz, 1H), 3.40- 3.59 (m, 2H), 7.54 (d, J = 8.00 Hz, 1H), 7.73 (d, J = 8.00 Hz, 1H), 10.52 | 482 | 480 | |
| | | (s, 1H) | | | |
| F-681 | | (400 MHz, DMSO-D6) 0.34-0.57 (m, 2H), 0.76-0.92 (m, 2H), 0.86 (s, 9H), 1.34 (d, J = 6.00 Hz, 2H), 1.38-1.49 (m, 1H), 1.78-2.01 (m, 4H), 2.13-2.26 (m, 2H), 2.17 (s, 3H), 2.27-2.46 (m, 3H), 2.31 (s, 3H), 2.88 (dd, J = 15.60, 6.80 Hz, 1H), 3.00 (dd, J = 15.60, 8.40 Hz, 1H), 3.44-3.61 (m, 2H), 7.92 (s, 1H), 8.16 (s, 1H), 10.56 (brs, 1H) | 482 | 480 | |

TABLE 337

| | | | MS | | Information |
|---|---|---|---|---|---|
| Example | Chemical structure | NMR | M + H | M − H | of structure |
| F-682 | | (400 MHz, CDCl3) 0.55-0.55 (m, 1H), 0.71-0.72 (m, 1H), 0.87 (s, 9H), 0.96-1.00 (m, 2H), 1.29 (d, J = 5.73 Hz, 2H), 1.61 (t, J = 4.19 Hz, 1H), 1.85 (d, J = 9.92 Hz, 1H), 2.00 (dd, J = 18.75, 9.92 Hz, 1H), 2.30- 2.41 (m, 10H), 3.05 (dd, J = 14.56, 4.63 Hz, 1H), 3.29-3.37 (m, 2H), 5.28 (s, 1H), 6.98 (d, J = 8.16 Hz, 1H), 7.11 (s, 1H), 7.77 (d, J = 8.38 Hz, 1H), 7.92 (s, 1H) | 501 | 499 | |
| F-683 | | (400 MHz, CDCl3) 0.33-0.37 (m, 1H), 0.40-0.45 (m, 1H), 0.79- 0.83 (m, 2H), 0.86 (s, 9H), 1.29- 1.35 (m, 1H), 1.37 (d, J = 5.73 Hz, 2H), 1.95-2.05 (m, 3H), 2.13- 2.23 (m, 1H), 2.26 (s, 3H), 2.31- 2.42 (m, 3H), 2.47-2.61 (m, 2H), 2.79 (dd, J = 15.44, 4.63 Hz, 1H), 3.13 (dd, J = 15.55, 10.03 Hz, 1H), 3.27-3.34 (m, 1H), 3.43-3.52 (m, 1H), 7.00 (d, J = 8.16 Hz, 1H), 7.11 | 501 | 499 | |
| | | (s, 1H), 7.95 (d, J = 8.38 Hz, 1H), | | | |
| | | 8.32 (s, 1H) | | | |
| F-684 | | (400 MHz, CDCl3) 0.33-0.37 (m, 1H), 0.40-0.45 (m, 1H), 0.79- 0.84 (m, 2H), 0.86 (s, 9H), 1.29- 1.35 (m, 1H), 1.37 (d, J = 5.95 Hz, 2H), 1.95-2.05 (m, 3H), 2.14- 2.23 (m, 1H), 2.26 (s, 3H), 2.31- 2.42 (m, 3H), 2.47-2.61 (m, 2H), 2.79 (dd, J = 15.55, 4.74 Hz, 1H), 3.14 (dd, J = 15.55, 10.03 Hz, 1H), 3.27-3.34 (m, 1H), 3.43-3.52 (m, 1H), 7.00 (dd, J = 8.31, 1.32 Hz, | 501 | 499 | |
| | | 1H), 7.11 (d, J = 1.32 Hz, 1H), 7.95 | | | |
| | | (d, J = 8.16 Hz, 1H), 8.32 (s, 1H) | | | |

TABLE 338

| | | | MS | | Information |
|---|---|---|---|---|---|
| Example | Chemical structure | NMR | M + H | M − H | of structure |
| F-685 | | (400 MHz, CDCl3) 0.31-0.36 (m, 1H), 0.46-0.50 (m, 1H), 0.80- 0.85 (m, 2H), 0.87 (s, 9H), 1.31- 1.37 (m, 1H), 1.37 (d, J = 5.95 Hz, 2H), 1.93-2.01 (m, 3H), 2.01 (s, 3H), 2.14-2.22 (m, 1H), 2.24 (s, 3H), 2.32-2.43 (m, 3H), 2.46- 2.54 (m, 1H), 2.57-2.64 (m, 1H), 2.74 (dd, J = 15.44, 3.97 Hz, 1H), 3.19 (dd, J = 15.22, 11.25 Hz, 1H), | 481 | 479 | |
| | | 3.30-3.38 (m, 1H), 3.45-3.54 (m, | | | |
| | | 1H), 6.90-6.94 (m, 2H), 7.34 (d, | | | |
| | | J = 8.60 Hz, 1H), 8.43 (s, 1H) | | | |
| F-686 | | (400 MHz, CDCl3) 0.33-0.48 (m, 2H), 0.77-0.94 (m, 11H), 1.20- 1.45 (m, 3H), 1.93-2.15 (m, 5H), 2.28 (s, 3H), 2.34-2.49 (m, 3H), 3.24-3.35 (m, 0.7H), 3.46-3.59 (m, 1.3H), 3.59-3.74 (m, 1H), 3.84- 4.03 (m, 2H), 4.07-4.23 (m, 1.3H), 4.37-4.47 (m, 0.7H), 7.01- 7.08 (m, 1H), 7.11-7.18 (m, 1H), 8.06-8.16 (m, 1.3H), 8.59 (s, | 512 | 510 | |
| | | 0.7H) | | | |
| F-687 | | (400 MHz, DMSO-D6) 0.35-0.48 (m, 2H), 0.73-0.91 (m, 2H), 0.86 (s, 9H), 1.30-1.43 (m, 1H), 1.35 (d, J = 5.60 Hz, 2H), 1.82-1.96 (m, 2H), 2.28 (s, 3H), 2.34-2.53 (m, 3H), 3.15-4.14 (m, 7H), 4.28 (s, 2H), 7.14 (d, J = 8.00 Hz, 1H), 7.32 (s, 1H), 7.50 (d, J = 8.00 Hz, 1H), 9.88 (s, 1H) | 528 | 526 | |

TABLE 339

| | | | MS | | Information |
|---|---|---|---|---|---|
| Example | Chemical structure | NMR | M + H | M − H | of structure |
| F-688 | | (400 MHz, DMSO-D6) 0.35-0.51 (m, 2H), 0.74-0.92 (m, 2H), 0.86 (s, 9H), 1.28-1.49 (m, 1H), 1.34 (d, J = 5.60 Hz, 2H), 1.81-1.97 (m, 2H), 2.23-2.57 (m, 5H), 2.27 (s, 3H), 3.04-4.16 (m, 9H), 7.12 (d, J = 8.00 Hz, 1H), 7.31 (s, 1H), 7.50 (dd, J = 12.80, 8.00 Hz, 1H), 9.67 (d, J = 4.80 Hz, 1H) | 570 | 568 | |
| F-689 | | (400 MHz, DMSO-D6) 0.33-055 (m, 2H), 0.73-0.91 (m, 2H), 0.86 (s, 9H), 1.29-1.47 (m, 5H), 1.63- 1.76 (m, 2H), 1.78-1.94 (m, 2H), 2.31-2.46 (m, 3H), 2.52 (s, 3H), 2.66 (s, 3H), 2.81 (dd, J = 15.12, 6.76 Hz, 1H), 2.92 (dd, J = 15.36, 8.60 Hz, 1H), 3.30-3.62 (m, 4H), 7.62 (d, J = 8.60 Hz, 1H), 8.30 (d, J = 8.36 Hz, 1H), 10.14 (s, 1H) | 468 | 466 | |
| F-690 | | (400 MHz, CDCl3) 0.88 (d, J = 6.62 Hz, 6H), 1.14 (d, J = 6.62 Hz, 3H), 1.33-1.52 (m, 6H), 1.80- 1.88 (m, 4H), 2.08 (s, 1H), 2.26- 2.34 (m, 5H), 2.68-2.76 (m, 3H), 3.06 (t, J = 11.80 Hz, 1H), 3.51 (s, 1H), 6.97 (d, J = 7.72 Hz, 1H), 7.11 (s, 1H), 8.04 (d, J = 8.16 Hz, 1H), 8.16 (s, 1H) | 475 | 473 | Single isomer (Stereo- chemistry of Cyclo- hexene ring: Undeter- mined) |
| F-691 | | (400 MHz, CDCl3) 0.85-0.88 (m, 6H), 1.12 (d, J = 8.38 Hz, 3H), 1.29- 1.36 (m, 3H), 1.54-1.56 (m, 3H), 1.75-1.81 (m, 3H), 1.99-2.07 (m, 2H), 2.29-2.38 (m, 5H), 2.67- 2.73 (m, 3H), 3.07 (t, J = 11.69 Hz, 1H), 3.47 (brs, 1H), 6.97 (d, J = 8.16 Hz, 1H), 7.10 (s, 1H), 7.81- 7.99 (m, 2H) | 475 | 473 | Mixture of three stereoi- somers (Mixture of F-693 to F-695) |

TABLE 340

| | | | MS | | Information |
|---|---|---|---|---|---|
| Example | Chemical structure | NMR | M + H | M − H | of structure |
| F-692 | | (400 MHz, DMSO-D6) 0.32-0.44 (m, 1H), 0.44-0.53 (m, 1H), 0.72- 0.84 (m, 10H), 1.12-1.24 (m, 2H), 1.28-1.35 (m, 1H), 1.36-1.46 (m, 1H), 1.77-1.98 (m, 4H), 2.11- 2.27 (m, 5H), 2.28-2.42 (m, 5H), 2.65-2.84 (m, 2H), 3.40-3.62 (m, 2H), 6.91-7.20 (m, 1H), 7.44- 7.70 (m, 1H), 9.37-9.61 (m, 1H), 11.93-12.15 (m, 1H) | 496 | 494 | |
| F-693 | | (400 MHz, CDCl3) 0.88 (d, J = 11.03 Hz, 6H), 1.18 (d, J = 6.84 Hz, 3H), 1.21-1.51 (m, 6H), 1.66-1.93 (m, 3H), 2.11 (t, J = 7.17 Hz, 1H), 2.26-2.28 (m, 4H), 2.43-2.51 (m, 2H), 2.72- 2.80 (m, 4H), 3.53 (t, J = 5.95 Hz, 1H), 7.02 (d, J = 8.38 Hz, 1H), 7.12 (s, 1H), 7.56 (s, 1H), 8.05 (d, J = 8.38 Hz, 1H) | 475 | 473 | Single isomer (Stereo- chemistry of Cyclo- hexene ring Undeter- mined) |
| F-694 | | (400 MHz, CDCl3) 0.88 (d, J = 19.19 Hz, 6H), 1.14 (t, J = 7.50 Hz, 3H), 1.33-1.41 (m, 3H), 1.55- 1.59 (m, 4H), 1.79-1.89 (m, 2H), 2.10 (dd, J = 8.60, 5.51 Hz, 1H), 2.27 (s, 4H), 2.46-2.48 (m, 2H), 2.61 (s, 1H), 2.76-2.87 (m, 3H), 3.47 (t, J = 8.49 Hz, 1H), 7.02 (d, J = 8.16 Hz, 1H), 7.13 (s, 1H), 7.59 (s, 19), 8.03 (d, J = 8.16 Hz, 1H) | 475 | 473 | Single isomer (Stereo- chemistry of Cyclo- hexene ring: Undeter- mined) |

TABLE 341

| | | | MS | | Information |
|---|---|---|---|---|---|
| Example | Chemical structure | NMR | M + H | M − H | of structure |
| F-695 | | (400 MHz, CDCl3) 0.89 (d, J = 6.62 Hz, 6H), 1.13 (d, J = 6.84 Hz, 3H), 1.32-1.38 (m, 3H), 1.55- 1.58 (m, 3H), 1.80 (t, J = 18.42 Hz, 3H), 2.04-2.10 (m, 2H), 2.27 (s, 3H), 2.36-2.40 (m, 2H), 2.69- 2.76 (m, 3H), 3.10 (dd, J = 11.69, 5.84 Hz, 1H), 3.50 (s, 1H), 7.01 (d, J = 8.38 Hz, 1H), 7.13 (s, 1H), 8.02- 8.05 (m, 2H) | 475 | 473 | Single isomer (Stereo- chemistry of Cyclo- hexene ring: Undeter- mined) |
| F-696 | | (400 MHz, CDCl3) 0.32-0.54 (m, 2H), 0.75-0.93 (m, 2H), 0.87 (s, 9H), 1.30-1.44 (m, 3H), 1.48- 1.72 (m, 3H), 1.74-2.10 (m, 4H), 2.00 (s, 3H), 2.23 (s, 3H), 2.32- 2.46 (m, 3H), 2.72 (dd, J = 13.72, 4.88 Hz, 1H), 2.96 (dd, J = 13.96, 10.40 Hz, 1H), 3.44-3.74 (m, 4H), 3.66 (s, 3H), 6.96 (d, J = 8.04 Hz, 1H), 7.28 (d, J = 8.40 Hz, 1H), 7.38 (s, 1H) | 497 | 495 | |
| F-697 | | (400 MHz, DMSO-D6) 0.72-0.83 (m, 9H), 1.11-1.22 (m, 2H), 1.28- 1.36 (m, 1H), 1.85-2.04 (m, 4H), 2.15-2.27 (m, 6H), 2.35-2.39 (m, 4H), 2.40-2.47 (m, 2H), 2.73- 2.86 (m, 2H), 3.41-3.51 (m, 1H), 3.60-3.77 (m, 1H), 7.02 (d, J = 7.60 Hz, 1H), 7.53 (d, J = 8.40 Hz, 1H), 9.51 (s, 1H), 12.13 (brs, 1H) | 524 | 522 | |

TABLE 342

| | | | MS | | Information |
|---|---|---|---|---|---|
| Example | Chemical structure | NMR | M + H | M − H | of structure |
| F-698 | | (400 MHz, CDCl3) 0.89 (d, J = 6.56 Hz, 6H), 1.25-1.57 (m, 6H), 1.79-2.09 (m, 5H), 2.27 (s, 3H), 2.37-2 41 (m, 4H), 2.75- 2.76 (m, 2H), 2.96-2.98 (m, 1H), 3.41-3.43 (m, 1H), 7.02 (d, J = 8.38 Hz, 1H), 7.13 (s, 1H), 7.89 (d, J = 5.29 Hz, 1H), 8.04 (d, J = 8.38 Hz, 1H) | 461 | 459 | Mixture of two stereo- isomers (Stereo- chemistry of Isopentyl group: Undeter- mined) |
| F-699 | | (400 MHz, CDCl3) 0.32-0.53 (m, 2H), 0.76-0.93 (m, 2H), 0.87 (s, 9H), 1.23-1.42 (m, 3H), 1.44- 1.70 (m, 3H), 1.75-2.11 (m, 4H), 1.95 (s, 3H), 2.18 (s, 3H), 2.32- 2.47 (m, 3H), 2.72 (dd, J = 13.76, 4.92 Hz, 1H), 2.94 (dd, J = 13.76, 10.40 Hz, 1H), 3.45-3.72 (m, 4H), 4.74 (s, 1H), 6.89 (d, J = 8.16 Hz, 1H), 6.99 (d, J = 8.16 Hz, 1H), 7.35 (s, 1H) | 483 | 481 | |
| F-700 | | (400 MHz, DMSO-D6) 0.34-0.44 (m, 1H), 0.44-0.53 (m, 1H), 0.73- 0.83 (m, 2H), 1.36-1.48 (m, 1H), 1.65-1.76 (m, 2H), 1.80-1.99 (m, 4H), 2.12-2.22 (m, 3H), 2.28 (s, 3H), 2.32-2.42 (m, 2H), 2.63- 2.87 (m, 2H), 3.39-3.59 (m, 2H), 7.05-7.12 (m, 1H), 7.13-7.21 (m, 3H), 7.23-7.30 (m, 3H), 7.42 (d, J = 8.40 Hz, 1H), 9.43 (s, 1H), 12.02 | 535 | 533 | |
| | | (brs, 1H) | | | |

TABLE 343

| | | | MS | | Information |
|---|---|---|---|---|---|
| Example | Chemical structure | NMR | M + H | M − H | of structure |
| F-701 | | (400 MHz, CDCl3) 0.29-0.40 (m, 1H), 0.61-0.70 (m, 1H), 0.74- 0.92 (m, 2H), 1.07-1.22 (m, 2H), 1.47-1.73 (m, 7H), 1.75-1.87 (m, 3H), 2.11-2.31 (m, 2H), 2.26 (s, 3H), 2.33-2.49 (m, 2H), 2.69 (t, J = 8.00 Hz, 2H), 2.85 (dd, J = 14.00, 4.80 Hz, 1H), 3.12 (dd, J = 14.00, 10.00 Hz, 1H), 3.59-3.68 (m, 1H), 7.00 (d, J = 8.40 Hz, 1H), 7.11 (s, | 549 | 547 | |
| | | 1H), 7.29 (d, J = 8.40 Hz, 2H), 7.37 | | | |
| | | (d, J = 8.40 Hz, 2H), 7.76 (s, 1H), | | | |
| | | 7.97 (d, J = 8.40 Hz, 1H) | | | |
| F-702 | | (400 MHz, CDCl3) 0.49-0.59 (m, 1H), 0.65-0.74 (m, 1H), 0.91- 1.04 (m, 2H), 0.97 (d, J = 6.88 Hz, 6H), 1.57-1.73 (m, 3H), 1.82- 2.02 (m, 3H), 2.28 (s, 3H), 2.59 (d, J = 7.24 Hz, 2H), 2.79 (dd, J = 14.52, 5.64 Hz, 1H), 3.03 (dd, J = 14.52, 8.88 Hz, 1H), 3.60-3.78 (m, 3H), 6.52 (s, 1H), 6.80-6 90 (m, 2H), 7.65 (s, 1H), 7.97-8.04 (m, 1H) | 469 | 467 | |
| F-703 | | (400 MHz, CDCl3) 0.54 (brs, 1H), 0.74 (brs, 1H), 0.82 (s, 9H), 0.99 (d, J = 8.16 Hz, 2H), 1.27 (d, J = 5.73 Hz, 2H), 1.64 (s, 1H), 1.85- 1.97 (m, 2H), 2.31-2.41 (m, 10H), 3.08 (dd, J = 9.92, 4.96 Hz, 1H), 3.33-3.36 (m, 2H), 5.29 (s, 1H), 6.81-6.83 (m, 2H), 7.67 (t, J = 8.27 Hz, 1H), 8.07 (s, 1H) | 485 | 483 | |

TABLE 344

| | | | MS | | Information |
|---|---|---|---|---|---|
| Example | Chemical structure | NMR | M + H | M − H | of structure |
| F-704 | | (400 MHz, CDCl3) 0.86 (s, 9H), 1.38 (d, J = 5.95 Hz, 2H), 2.00-2.09 (m, 2H), 2.26 (s, 3H), 2.35-2.45 (m, 3H), 2.88-2.91 (m, 2H), 3.07- 3.10 (m, 2H), 3.39-3.48 (m, 1H), 4.04 (brs, 2H), 4.34 (brs, 2H), 7.00 (d, J = 7.28 Hz, 1H), 7.13 (s, 1H), 7.77 (s, 1H), 8.06 (d, J = 8.16 Hz, 1H) | 477 | 475 | |
| F-705 | | (400 MHz, CDCl3) 0.24-0.34 (m, 1H), 0.42-0.52 (m, 1H), 0.78- 0.96 (m, 2H), 0.96-1.02 (m, 9H), 1.50-1.54 (m, 1H), 1.59-1.71 (m, 2H), 1.80-2.00 (m, 5H), 2.29 (s, 3H), 2.73-2.84 (m, 1H), 2.93- 3.05 (m, 1H), 3.64-3.75 (m, 3H), 4.20-4.28 (m, 2H), 6.81-6.93 (m, 2H), 7.50-7.56 (m, 1H), 7.75 (brs, 1H), 7.97-8.05 (m, 1H) | 565 | 563 | |
| F-706 | | (400 MHz, CDCl3) 0.45-0.54 (m, 1H), 0.74-0.79 (m, 6H), 0.79- 0.83 (m, 6H), 0.93-1.02 (m, 3H), 1.40-1.48 (m, 4H), 2.04-2.17 (m, 4H), 2.21-2.26 (m, 4H), 2.27- 2.36 (m, 3H), 2.36-2.48 (m, 1H), 2.52-2.68 (m, 2H), 2.71-2.81 (m, 1H), 2.92-3.02 (m, 1H), 3.44- 3.60 (m, 1H), 4.66-4.81 (m, 1H), 6.74-6.86 (m, 2H), 7.67 (brs, 1H), 7.79-7.87(m, 1H) | 513 | 511 | |

TABLE 345

| | | | MS | | Information of |
|---|---|---|---|---|---|
| Example | Chemical structure | NMR | M + H | M − H | structure |
| F-707 | | (400 MHz, CDCl3) 0.76 (s, 6H), 0.79-0.84 (m, 6H), 1.36-1.48 (m, 2H), 2.05-2.17 (m, 3H), 2.20- 2.33 (m, 7H), 2.33-2.51 (m, 2H), 2.57-2.73 (m, 2H), 2.75-2.86 (m, 1H), 2.98-3.09 (m, 1H), 3.58- 3.73 (m, 1H), 4.66-4.78 (m, 1H), 6.80-6.88 (m, 2H), 7.42 (brs, 1H), 7.93-7.99 (m, 1H) | 541 | 539 | |
| F-708 | | (400 MHz, DMSO-D6) 0.73-0.78 (m, 7H), 0.78-0.82 (m, 6H), 0.99- 1.12 (m, 4H), 1.29-1.36 (m, 2H), 1.38-1.48 (m, 1H), 1.87-2.01 (m, 2H), 2.15-2.22 (m, 1H), 2.24- 2.27 (m, 3H), 2.31-2.43 (m, 2H), 2.62-2.80 (m, 2H), 2.87-3.01 (m, 3H), 3.42-3.53 (m, 1H), 3.55- 3.65 (m, 1H), 7.05-7.10 (m, 1H), 7.26 (brs, 1H), 7.37-7.42 (m, 1H), 9.47 (brs, 1H), 11.86-12.34 (m, 1H) | 529 | 527 | |
| F-709 | | (400 MHz, CDCl3) 0.45-0.53 (m, 1H), 0.72-0.79 (m, 8H), 0.79- 0.84 (m, 6H), 0.95-1.01 (m, 2H), 1.37-1.50 (m, 4H), 1.52-1.63 (m, 2H), 1.72-1.92 (m, 2H), 2.17- 2.24 (m, 1H), 2.24-2.27 (m, 3H), 2.27-2.35 (m, 1H), 2.35-2.47 (m, 1H), 2.51-2.68 (m, 2H), 2.70- 2.77 (m, 1H), 2.89-2.99 (m, 1H), 3.37-3.45 (m, 1H), 3.57-3.62 (m, 2H), 4.70-4.80 (m, 1H), 6.77- 6.86 (m, 2H), 7.53 (brs, 1H), 7.89 | 499 | 497 | |
| | | (t, J = 8.00 Hz, 1H) | | | |

TABLE 346

| | | | MS | | Information |
|---|---|---|---|---|---|
| Example | Chemical structure | NMR | M + H | M − H | of structure |
| F-710 | | (400 MHz, CDCl3) 0.79 (s, 6H), 0.83 (d, J = 6.80 Hz, 6H), 1.45-1.49 (m, 4H), 1.78-1.91 (m, 2H), 2.20- 2.27 (m, 2H), 2.29 (s, 3H), 2.36- 2.52 (m, 2H), 2.60-2.75 (m, 2H), 2.77-2.87 (m, 1H), 2.97-3.08 (m, 1H), 3.56-3.67 (m, 4H), 4.68- 4.81 (m, 1H), 6.81-6.91 (m, 2H), 7.45 (brs, 1H), 7.96-8.04 (m, 1H) | 527 | 525 | |
| F-711 | | (400 MHz, CDCl3) 0.34-0.39 (m, 1H), 0.43-0.48 (m, 1H), 0.81 (dt, J = 8.26, 4.30 Hz, 2H), 0.85 (s, 9H), 1.31-1.36 (m, 1H), 1.37 (d, J = 5.58 Hz, 2H), 1.52-1.66 (m, 3H), 1.76 (dd, J = 18.72, 9.65 Hz, 1H), 1.96-2.18 (m, 5H), 2.21- 2.29 (m, 1H), 2.33-2.40 (m, 5H), 2.53 (dd, J = 13.84, 6.16 Hz, 1H), 2.60 (dd, J = 13.96, 9.77 Hz, 1H), 3.45-3.60 (m, 2H), 4.25 (q, J = 7.68 Hz, 1H), 6.06 (brd, | 431 | 429 | |
| | | J = 7.91 Hz, 1H) | | | |
| F-712 | | (400 MHz, CDCl3) 0.34-0.39 (m, 1H), 0.43-0.48 (m, 1H), 0.80- 0.85 (m, 2H), 0.86 (s, 9H), 1.22- 1.39 (m, 4H), 1.94-2.14 (m, 4H), 1.97 (s, 6H), 2.32-2.42 (m, 5H), 2.47 (dd, J = 14.07, 6.40 Hz, 1H), 2.56 (dd, J = 14.19, 8.37 Hz, 1H), 3.47-3.55 (m, 2H), 6.09 (brs, 1H) | 443 | 441 | |

TABLE 347

| | | | MS | | Information |
|---|---|---|---|---|---|
| Example | Chemical structure | NMR | M + H | M − H | of structure |
| F-713 | | (400 MHz, DMSO-D6) 0.45-0.53 (m, 1H), 0.75-0.84 (m, 14H), 0.89- 0.96 (m, 2H), 1.39-1.45 (m, 2H), 1.53-1.60 (m, 1H), 1.84-1.96 (m, 2H), 2.03-2.11 (m, 2H), 2.22- 2.27 (m, 4H), 2.58-2.70 (m, 4H), 2.70-2.83 (m, 1H), 4.79-4.94 (m, 1H), 7.04-7.10 (m, 1H), 7.25 (brs, 1H), 7.36 (d, J = 8.40 Hz, 1H), 9.30- 9.37 (m, 1H), 11.99 (brs, 1H) | 529 | 527 | |
| F-714 | | (400 MHz, CDCl3) 0.69-0.78 (m, 6H), 0.78-0.85 (m, 6H), 1.19- 1.30 (m, 1H), 1.30-1.46 (m, 3H), 1.91-2.21 (m, 4H), 2.21-2.29 (m, 3H), 2.33-2.56 (m, 4H), 2.77- 2.97 (m, 2H), 2.99-3.14 (m, 1H), 3.15-3.42 (m, 2H), 3.54-3.76 (m, 3H), 6.89-7.03 (m, 1H), 7.08- 7.14 (m, 1H), 7.58-7.80 (m, 1H), 8.07-8.25 (m, 1H) | 565 | 563 | Racemic form (Stereo- chemistry of asym- metric point on Cyclo- propane Mixture of R-isomer and S-iso- mer) |
| E-77 | | (400 MHz, DMSO-D6) 0.45-0.58 (m, 1H), 0.63-0.75 (m, 1H), 0.90- 1.02 (m, 2H), 1.08 (s, 9H), 1.71- 1.82 (m, 1H), 1.90-2.05 (m, 2H), 2.18-2.33 (m, 2H), 2.87 (dd, J = 15.60, 6.40 Hz, 1H), 2.97 (dd, J = 15.60, 8.80 Hz, 1H), 3.55-3.67 (m, 1H), 7.17-7.23 (m, 1H), 7.39 (s, 1H), 7.43 (dd, J = 10.80, 2.40 Hz, 1H), 7.83 (dd, J = 8.80, 8.00 Hz, 1H), 9.92 (s, 1H), 12.16 (brs, 1H) | 554 | 552 | |

TABLE 348

| | | | MS | | Information |
|---|---|---|---|---|---|
| Example | Chemical structure | NMR | M + H | M − H | of structure |
| E-78 | | (400 MHz, DMSO-D6) 0.42-0.54 (m, 1H), 0.60-0.71 (m, 1H), 0.86- 1.02 (m, 2H), 0.95 (s, 9H), 1.67- 1.77 (m, 1H), 1.98 (dd, J = 14.40, 7.60 Hz, 2H), 2.16-2.33 (m, 2H), 2.76 (s, 2H), 2.85 (dd, J = 15.60, 6.40 Hz, 1H), 2.95 (dd, J = 15.60, 8.80 Hz, 1H), 3.52-3.64 (m, 1H), 6.74 (s, 1H), 7.36 (dd, J = 8.80, 2.00 Hz, 1H), 7.60 (d, J = 2.00 Hz, 1H), 7.63 (d, J = 8.80 Hz, 1H), 9.68 (s, 1H), 12.30 (brs, 1H) | 534 | 532 | |
| E-79 | | (400 MHz, CDCl3) 0.57-0.62 (m, 1H), 0.72-0.77 (m, 1H), 0.97- 1.06 (m, 2H), 1.01 (s, 9H), 1.65- 1.72 (m, 1H), 2.11-2.26 (m, 2H), 2.43-2.48 (m, 2H), 2.72 (s, 2H), 2.84 (dd, J = 14.78, 5.29 Hz, 1H) 3.10 (dd, J = 14.78, 9.70 Hz, 1H), 3.75-3.82 (m, 1H), 6.41 (s, 1H), 7.19 (dd, J = 8.82, 2.43 Hz, 1H), 7.33 (d, J = 2.43 Hz, 1H), 7.82 (s, 1H), 8.21 (d, J = 9.04 Hz, 1H) | 534 | 532 | |
| E-80 | | (400 MHz, CDCl3) 0.60-0.65 (m, 1H), 0.77-0.83 (m, 1H), 1.03- 1.08 (m, 2H), 1.14 (s, 9H), 1.68- 1.75 (m, 1H), 2.09-2.26 (m, 2H), 2.44-2.48 (m, 2H), 2.83 (dd, J = 15.11, 5.18 Hz, 1H), 3.07 (dd, J = 15.11, 9.59 Hz, 1H), 3.77-3.84 (m, 1H), 6.88 (s, 1H), 7.06-7.10 (m, 2H), 7.60 (s, 1H), 8.16 (t, J = 8.82 Hz, 1H) | 554 | 552 | |

TABLE 349

| | | | MS | | Information |
|---|---|---|---|---|---|
| Example | Chemical structure | NMR | M + H | M − H | of structure |
| F-715 | | (400 MHz, CDCl3) 0.29-0.37 (m, 1H), 0.54-0.63 (m, 1H), 0.75- 0.83 (m, 2H), 0.85 (s, 9H), 1.22- 1.37 (m, 3H), 1.79-1.91 (m, 1H), 1.93-2.03 (m, 1H), 2.04-2.13 (m, 2H), 2.23-2.34 (m, 5H), 2.36- 2.53 (m, 3H), 2.69-2.89 (m, 2H), 3.26-3.37 (m, 1H), 3.67-3.78 (m, 1H), 7.02 (d, J = 8.38 Hz, 1H), 7.14 (s, 1H), 7.45 (brs, 1H), 8.03 (d, J = 8.38 Hz, 1H) | 500 | 498 | |
| F-716 | | (400 MHz, DMSO-D6) 0.33-0.54 (m, 2H), 0.71-0.83 (m, 8H), 1.30- 1.49 (m, 3H), 1.78-2.00 (m, 4H), 2.06-2.44 (m, 8H), 2.65-2.89 (m, 2H), 3.06 (s, 2H), 3.38-3.60 (m, 2H), 4.42 (brs, 1H), 7.09 (d, J = 7.86 Hz, 1H), 7.28 (s, 1H), 7.42 (d, J = 8.32 Hz, 1H), 9.46 (s, 1H) | 517 | 515 | |
| F-717 | | (400 MHz, CDCl3) 0.25-0.39 (m, 1H), 0.55-0.68 (m, 1H), 0.79- 0.82 (m, 2H), 0.84 (s, 9H), 1.24- 1.37 (m, 3H), 1.78-1.88 (m, 1H), 1.88-2.00 (m, 1H), 2.00-2.13 (m, 2H), 2.23-2.34 (m, 2H), 2.34- 2.55 (m, 3H), 2.72-2.91 (m, 2H), 3.25-3.38 (m, 1H), 3.74 (dd, J = 10.81, 3.97 Hz, 1H), 7.00-7.11 (m, 2H), 7.56 (brs, 1H), 8.07 (t, J = 8.71 Hz, 1H) | 505 | 503 | |

TABLE 350

| | | | MS | | Information |
|---|---|---|---|---|---|
| Example | Chemical structure | NMR | M + H | M − H | of structure |
| F-718 | | (400 MHz, CDCl3) 0.26-0.37 (m, 1H), 0.53-0.63 (m, 1H), 0.80 (ddd, J = 11.03, 5.07, 2.98 Hz, 2H), 0.85 (s, 9H), 1.22-1.38 (m, 3H), 1.77- 1.88 (m, 1H), 1.91-2.02 (m, 1H), 2.03-2.14 (m, 2H), 2.30 (t, J = 7.39 Hz, 2H), 2.36-2.52 (m, 3H), 2.76 (dd, J = 14.56, 5.95 Hz, 1H), 2.85 (dd, J = 14.56, 9.04 Hz, 1H), 3.27-3.37 (m, 1H), 3.66- 3.78 (m, 1H), 7.20 (dd, J = 9.04, | 521 | 519 | |
| | | 2.21 Hz, 1H), 7.34 (d, J = 2.21 Hz, | | | |
| | | 1H), 7.50 (brs, 1H), 8.18 (d, | | | |
| | | J = 9.04 Hz, 1H) | | | |
| F-719 | | (400 MHz, CDCl3) 0.43 (tdd, J = 5.60, 4.12, 0.12 Hz, 2H), 0.83 (ddd, J = 9.36, 5.09, 3.47 Hz, 2H), 0.87 (s, 9H), 1.33-1.49 (m, 3H), 1.98-2.07 (m, 2H), 2.29 (s, 3H), 2.33-2.44 (m, 3H), 2.92 (t, J = 7.28 Hz, 2H), 3.07 (t, J = 7.17 Hz, 2H), 3.51 (ddd, J = 19.25, 8.73, | 429 | 427 | |
| | | 6.53 Hz, 1H), 7.05 (d, J = 8.79 Hz, | | | |
| | | 1H), 7.17 (d, J = 0.92 Hz, 1H), 7.85 | | | |
| | | (brs, 1H), 8.20 (d, J = 8.55 Hz, 1H) | | | |
| F-720 | | (400 MHz, CDCl3) 0.43 (td, J = 5.43, 4.62 Hz, 2H), 0.84 (ddd, J = 9.42, 5.03, 3.29 Hz, 2H), 0.87 (s, 9H), 1.33-1.41 (m, 3H), 1.98- 2.07 (m, 2H), 2.36-2.43 (m, 3H), 2.94 (t, J = 6.94 Hz, 2H), 3.07 (t, J = 6.94 Hz, 2H), 3.51 (tt, J = 10.17, 4.16 Hz, 1H), 7.23 (dd, J = 8.90, | 449 | 447 | |
| | | 2.43 Hz, 1H), 7.36 (d, J = 2.31 Hz, | | | |
| | | 1H), 8.00 (brs, 1H), 8.34 (d, | | | |
| | | J = 9.02 Hz, 1H) | | | |

TABLE 351

| | | | MS | | Information |
|---|---|---|---|---|---|
| Example | Chemical structure | NMR | M + H | M − H | of structure |
| F-721 | | (400 MHz, CDCl3) 0.42 (td, J = 5.72, 4.16 Hz, 2H), 0.83 (ddd, J = 9.13, 4.62, 3.01 Hz, 2H), 0.87 (s, 9H), 1.32-1.40 (m, 3H), 1.99- 2.05 (m, 2H), 2.16 (s, 3H), 2.27 (s, 3H), 2.37-2.43 (m, 3H), 2.90 (t, J = 7.05 Hz, 2H), 3.07 (t, J = 7.05 Hz, 2H), 3.51 (ddd, J = 19.30, 8.90, | 409 | 407 | |
| | | 6.70 Hz, 1H), 6.97 (s, 1H), 6.99 (d, | | | |
| | | J = 8.79 Hz, 1H), 7.50 (brs, 1H), | | | |
| | | 7.64 (d, J = 7.86 Hz, 1H) | | | |
| F-722 | | (400 MHz, CDCl3) 0.43-0.44 (m, 1H), 0.51-0.54 (m, 1H), 0.86- 0.88 (m, 10H), 0.97-1.01 (m, 2H), 1.28-1.35 (m, 5H), 2.03-2.09 (m, 5H), 2.37-2.40 (m, 5H), 2.74- 2.78 (m, 1H), 2.84-2.93 (m, 2H), 3.50-3.53 (m, 1H), 3.64-3.67 (m, 1H) | 481 | 479 | |
| F-723 | | (400 MHz, CDCl3) 0.30 (d, J = 3.97 Hz, 2H), 0.76-0.80 (m, 2H), 0.88 (s, 9H), 1.25 (dd, J = 10.92, 5.62 Hz, 1H), 1.39 (d J = 5.95 Hz, 2H), 1.93-2.05 (m, 4H), 2.30-2.40 (m, 5H), 2.66 (dd, J = 14.89, 5.62 Hz, 1H), 2.82 (dd, J = 14.89, 9.59 Hz, 1H), 3.47-3.50 (m, 2H), 7.46 (t, J = 7.72 Hz, 2H), 7.57 (t, J = 7.50 Hz, 1H), 7.95 (d, J = 4.30 Hz, 2H) | 517 | 515 | |

TABLE 352

| | | | MS | | Information |
|---|---|---|---|---|---|
| Example | Chemical structure | NMR | M + H | M − H | of structure |
| F-724 | | (400 MHz, DMSO-D6) 0.34-0.54 (m, 2H), 0.73-0.91 (m, 8H), 1.32- 1.46 (m, 3H), 1.77-2.00 (m, 4H), 2.10-2.28 (m, 2H), 2.30-2.45 (m, 3H), 2.66-2.87 (m, 2H), 2.97 (s, 2H), 3.23 (s, 3H), 3.40-3.58 (m, 2H), 4.45 (s, 2H), 5.27 (brs, 1H), 7.21 (d, J = 8.55 Hz, 1H), 7.38 (s, 1H), 7.51 (d, J = 8.09 Hz, 1H), 9.50 (s, 1H), 12.08 (brs, 1H) | 547 | 545 | |
| F-725 | | (400 MHz, DMSO-D6) 0.32-0.54 (m, 2H), 0.72-0.85 (m, 8H), 1.33- 1.45 (m, 3H), 1.79-1.99 (m, 4H), 2.09-2.24 (m, 2H), 2.26 (s, 3H), 2.30-2.45 (m, 3H), 2.65-2.85 (m, 2H), 2.97 (s, 2H), 3.23 (s, 3H), 3.38-3.59 (m, 2H), 7.09 (d, J = 7.17 Hz, 1H), 7.28 (s, 1H), 7.42 (d, J = 8.32 Hz, 1H), 9.46 (1H, s), 12.08 (1H, s) | 531 | 529 | |
| F-726 | | (400 MHz, DMSO-D6) 0.31-0.56 (m, 2H), 0.69-0.90 (m, 8H), 0.88 (s, 4H), 1.32-1.50 (m, 3H), 1.78- 1.98 (m, 4H), 2.04-2.26 (m, 2H), 2.29-2.45 (m, 3H), 2.63-2.89 (m, 2H), 3.05 (s, 2H), 3.40-3.61 (m, 2H), 4.36-4.52 (m, 3H), 5.27 (brs, 1H), 7.21 (d, J = 7.40 Hz, 1H), 7.38 (s, 1H), 7.51 (d, J = 8.09 Hz, 1H), 9.51 (s, 1H) | 533 | 531 | |

TABLE 353

| | | | MS | | Information |
|---|---|---|---|---|---|
| Example | Chemical structure | NMR | M + H | M − H | of structure |
| F-727 | | (400 MHz, DMSO-D6) 0.34-0.53 (m, 2H), 0.75-0.84 (m, 2H), 1.07 (s, 6H), 1.37-1.46 (m, 1H), 1.60- 1.67 (m, 2H), 1.82-1.99 (m, 4H), 2.11-2.24 (m, 2H), 2.27 (s, 3H), 2.29-2.41 (m, 3H), 2.65-2.85 (m, 2H), 3.27-3.31 (m, 1H), 3.38- 3.58 (m, 2H), 7.09 (d, J = 7.86 Hz, 1H), 7.28 (s, 1H ), 7.42 (d, | 531 | 529 | |
| | | J = 8.32 Hz, 1H), 9.46 (s, 1H), 12.07 | | | |
| | | (s, 1H) | | | |
| F-728 | | (400 MHz, DMSO-D6) 0.34-0.53 (m, 2H), 0.74-0.83 (m, 2H), 1.03 (s, 6H), 1.35-1.45 (m, 1H), 1.56- 1.63 (m, 2H), 1.77-1.98 (m, 4H), 2.09-2.36 (m, 8H), 2.47-2.57 (m, 3H), 2.65-2.86 (m, 2H), 3.39- 3.55 (m, 2H), 7.09 (d, J = 8.09 Hz, 1H), 7.28 (s, 1H), 7.36-7.46 (m, 2H), 9.45 (s, 1H), 12.07 (brs, 1H) | 544 | 542 | |
| F-729 | | (400 MHz, CDCl3) 0.43-0.49 (m, 2H), 0.80-0.90 (m, 11H), 1.24 (ddd, J = 14.81, 8.16, 4.73 Hz, 2H), 1.40 (dq, J = 11.89, 3.16 Hz, 1H), 1.59-1.68 (m, 2H), 2.29 (s, 3H), 2.68 (t, J = 7.66 Hz, 2H), 2.94 (t, J = 7.25 Hz, 2H), 3.09 (t, J = 7.25 Hz, 2H), 7.05 (dd, J = 8.46, 2.01 Hz, | 403 | 401 | |
| | | 1H), 7.17 (d, J = 1.21 Hz, 1H), 7.88 | | | |
| | | (s, 1H), 8.20 (d, J = 8.46 Hz, 1H) | | | |

TABLE 354

| | | | MS | | Information |
|---|---|---|---|---|---|
| Example | Chemical structure | NMR | M + H | M − H | of structure |
| F-730 | | (400 MHz, CDCl3) 0.53-0.60 (m, 2H), 0.81-0.90 (m, 2H), 0.84 (s, 3.6H), 0.85 (s, 5.4H), 1.35 (d, J = 6.70 Hz, 1.2H), 1.37 (d, J = 7.17 Hz, 0.8H), 1.42-1.55 (m, 2H), 1.93 (ddd, J = 10.87, 8.21, 1.73 Hz, 0.8H), 2.16 (ddd, J = 10.40, 7.98, 1.50 Hz, 1.2H), 2.29 (s, 3H), | 459 | 457 | Mixture of two stereoi- somers (Stereo- chemistry, on Cyclobu- tane ring: |
| | | 2.36-2.49 (m, 2H), 2.77-2.83 (m, | | | Mixture of |
| | | 1H), 2.91-2.98 (m, 2H), 3.08 (t, | | | Cis-isomer |
| | | J = 7.05 Hz, 1.2H), 3.11 (t, | | | and Trans- |
| | | J = 7.17 Hz, 0.8H), 3.77 (s, 0.8H), | | | isomer) |
| | | 3.95 (s, 1.2H), 7.06 (dd, J = 8.55, | | | |
| | | 1.85 Hz, 1H), 7.17 (s, 1H), 7.87 | | | |
| | | (brs, 0.6H), 7.89 (brs, 0.4H), 8.21 | | | |
| | | (d, J = 8.32 Hz, 1H) | | | |
| F-731 | | (400 MHz, DMSO-D6) 0.35-0.55 (m, 2H), 0.72-0.84 (m, 8H), 1.33 (d, J = 5.55 Hz, 2H), 1.37-1.47 (m, 1H), 1.78-1.99 (m, 4H), 2.12- 2.26 (m, 2H), 2.30-2.44 (m, 3H), 2.77-3.00 (m, 2H), 3.01-3.10 (m, 2H), 3.42-3.59 (m, 2H), 4.39- 4.46 (m, 1H), 7.84 (dd, J = 8.55, 1.85 Hz, 1H), 7.89-7.96 (m, 2H), 9.66 (s, 1H) | 547 | 545 | |

TABLE 355

| | | | MS | | Information |
|---|---|---|---|---|---|
| Example | Chemical structure | NMR | M + H | M − H | of structure |
| F-732 | | (400 MHz, CDCl3) 0.59 (td, J = 5.61, 3.70 Hz, 0.4H), 0.66 (td, J = 5.66, 4.16 Hz, 1.6H), 0.86 (s, 7.2H), 0.87 (s, 1.8H), 0.84-0.93 (m, 2H), 1.38 (d, J = 6.94 Hz, 0.4H), 1.43 (d, J = 5.78 Hz, 1.6H), 1.51- 1.55 (m, 1H), 2.00-2.06 (m, 2.4H), 2.29 (s, 24H), 2.26-2.36 (m, | 445 | 443 | Mixture of two stereoi- somers (Stereo- chemistry on Cyclobu- tane ring: |
| | | 1.4H), 2.36 (s, 0.6H), 2.44-2.52 | | | Mixture of |
| | | (m, 0.4H), 2.73-2.83 (m, 0.2H), | | | Cis-isomer |
| | | 2.94-3.00 (m, 3.6H), 3.08-3.14 | | | and Trans- |
| | | (m, 2H), 7.06 (d, J = 8.55 Hz, 1H), | | | isomer) |
| | | 7.18 (s, 1H), 7.81 (brs, 0.2H), 7.84 | | | |
| | | (brs, 0.8H), 8.21 (d, J = 8.32 Hz, 1H) | | | |
| F-733 | | (400 MHz, CDCl3) 0.86 (s, 9H), 1.35 (d, J = 6.70 Hz, 2H), 1.86 (ddd, J = 19.07, 9.13, 2.89 Hz, 2H), 2.29 (s, 3H), 2.34-2.42 (m, 1H), 2.48 (ddd, J = 15.72, 8.09, 2.77 Hz, 2H), 2.84 (t, J = 7.17 Hz, 2H), 3.06 (t, | 389 | 387 | |
| | | J = 7.28 Hz, 2H), 3.35-3.44 (m, | | | |
| | | 1H), 5.85 (s, 1H), 7.06 (dd, J = 8.55, | | | |
| | | 1.39 Hz, 1H), 7.17 (brs, 1H), 7.70 | | | |
| | | (s, 1H), 8.20 (d, J = 8.32 Hz, 1H) | | | |
| F-734 | | (400 MHz, CDCl3) 0.60-0.62 (m, 2H), 0.92-0.95 (m, 2H), 1.47- 1.51 (m, 1H), 1.78 (t, J = 3.09 Hz, 6H), 2.07 (s, 3H), 2.13 (d, J = 3.09 Hz, 6H), 2.28 (s, 3H), 2.92 (t, J = 7.17 Hz, 2H), 3.08 (t, J = 7.06 Hz, 2H), 7.05 (d, J = 8.38 Hz, 1H), 7.16 (s, 1H), 7.92 (s, 1H), 8.20 (d, J = 8.38 Hz, 1H) | 439 | 437 | |

TABLE 356

| | | | MS | | Information |
|---|---|---|---|---|---|
| Example | Chemical structure | NMR | M + H | M − H | of structure |
| F-735 | | (400 MHz, CDCl3) 0.58-0.61 (m, 2H), 0.90-0.92 (m, 2H), 1.43- 1.47 (m, 1H), 1.78 (d, J = 2.87 Hz, 6H), 2.06 (s, 3H), 2.11 (d, J = 2.87 Hz, 6H), 2.34 (s, 3H), 2.64 (t, J = 7.50 Hz, 2H), 2.98 (t, J = 7.50 Hz, 2H), 3.83 (d, J = 5.29 Hz, 3H), 4.39 (d, J = 5.73 Hz, 2H), 6.20 (s, 1H), 6.69-6.71 (m, 2H), 7.11 (d, J = 7.50 Hz, 1H) | 449 | 447 | |
| F-736 | | (400 MHz, CDCl3) 0.36 (dd, J = 9.02, 3.70 Hz, 1H), 0.47 (dd, J = 11.21, 5.90 Hz, 1H), 0.81-0.85 (m, 11H), 1.11-1.18 (m, 2H), 1.32- 1.37 (m, 4H), 1.54-1.57 (m, 1H), 1.95-2.09 (m, 5H), 2.35-2.41 (m, 5H), 2.59 (dd, J = 13.76. 5.20 Hz, H), 2.70 (dd, J = 13.87, 10.17 Hz, 1H), 2.89-2.96 (m, 1H), 3.10- 3.17 (m, 1H), 3.23-3.27 (m, 2H), 3.50-3.56 (m, 2H), 3.87-3.88 (m, 2H), 6.39 (t, J = 6.01 Hz, 1H) | 475 | 473 | |
| F-737 | | (400 MHz, CDCl3) 0.49 (td, J = 5.61, 4.16 Hz, 2H), 0.82-0.86 (m, 2H), 0.85 (s, 9H), 1.34 (d, J = 6.94 Hz, 2H), 1.39-1.44 (m, 1H), 1.93 (s, 3H), 2.18-2.24 (m, 2H), 2.29 (s, 3H), 2.29-2.33 (m, 2H), 2.38 (td, J = 9.02, 2.54 Hz, 2H), 2.45-2.58 (m, 1H), 2.89-2.96 (m, 2H), 3.06 (t, J = 7.28 Hz, 2H), 3.98 (t, J = 7.05 Hz, 2H), 7.06 (d, | 515 | 513 | Mixture of two stereoi- somers (Stereo- chemistry on Cyclobu- tane ring: Mixture of Cis-isomer |
| | | J = 8.32 Hz, 1H), 7.17 (s, 1H), 7.87 | | | and Trans- |
| | | (brs, 1H), 8.21 (d, J = 8.09 Hz, 1H) | | | isomer) |

TABLE 357

| | | | | | Informa- |
|---|---|---|---|---|---|
| Exam- | | | MS | | tion of |
| ple | Chemical structure | NMR | M + H | M − H | structure |
| F-738 | | (400 MHz, CDCl3) 0.47 (td, J = 5.64, 4.16 Hz, 2H), 0.89 (ddt, J = 19.54, 10.95, 4.33 Hz, 2H), 1.20- 1.48 (m, 3H), 1.68 (dt, J = 16.39, 6.95 Hz, 2H), 2.08 (dt, J = 14.64, 4.63 Hz, 2H), 2.72 (t, J = 7.66 Hz, | 365 | 363 | |
| | | 2H), 2.87 (t, J = 7.05 Hz, 2H), 3.09 | | | |
| | | (t, J = 7.05 Hz, 2H), 4.98 (dtt, | | | |
| | | J = 19.79, 8.03, 2.69 Hz, 2H), 5.25 | | | |
| | | (d, J = 10.88 Hz, 1H), 5.78 (ddt, | | | |
| | | J = 21.83, 12.29, 4.48 Hz, 2H), 6.68 | | | |
| | | (dd, J = 17.73, 10.88 Hz, 1H), 7.14 | | | |
| | | (d, J = 7.66 Hz, 1H), 7.25-7.27 (m, | | | |
| | | 1H), 7.38 (d, J = 8.06 Hz, 1H), 7.60 | | | |
| | | (s, 1H), 7.82 (s, 1H) | | | |
| F-739 | | (400 MHz, DMSO-D6) 0.34-0.53 (m, 2H), 0.74-0.83 (m, 2H), 1.07 (s, 6H), 1.35-1.47 (m, 1H), 1.59- 1.68 (m, 2H), 1.80-1.98 (m, 4H), 2.12-2.24 (m, 2H), 2.24-2.41 (m, 3H), 2.69-2.87 (m, 2H), 3.40- 3.60 (m, 2H), 4.45 (s, 2H), 5.28 (brs, 1H), 7.21 (d, J = 8.09 Hz, 1H), 7.38 (s, 1H), 7.51 (d, J = 8.32 Hz, | 547 | 545 | |
| | | 1H), 9.51 (s, 1H), 12.07 (brs, 1H) | | | |
| F-740 | | (400 MHz, CDCl3) 0.66-0.67 (m, 2H), 0.89-0.94 (m, 2H), 1.36 (s, 9H), 1.56 (s, 1H), 2.29 (s, 3H), 2.96 (t, J = 7.06 Hz, 2H), 3.16 (t, J = 7.28 Hz, 2H), 6.56 (d, J = 1.10 Hz, 1H), 7.06 (d, J = 8.38 Hz, 1H), 7.18 (s, 1H), 7.74 (s, 1H), 8.18 (d, J = 8.38 Hz, 1H) | 478 | 476 | |

TABLE 358

| | | | MS | | Information |
|---|---|---|---|---|---|
| Example | Chemical structure | NMR | M + H | M − H | of structure |
| F-741 | | (400 MHz, CDCl3) 0.60-0.63 (m, 2H), 0.87-0.89 (m, 2H), 1.36 (s, 9H), 1.48-1.51 (m, 1H), 2.34 (s, 3H), 2.67 (t, J = 7.50 Hz, 2H), 3.06 (t, J = 7.50 Hz, 2H), 3.83 (s, 3H), 4.39 (d, J = 5.73 Hz, 2H), 6.11 (s, 1H), 6.55 (d, J = 1.10 Hz, 1H), 6.70- 6.72 (m, 2H), 7.12 (d, J = 7.50 Hz, 1H) | 488 | 486 | |
| F-742 | | (400 MHz, CDCl3) 0.49 (td, J = 5.72, 4.16 Hz, 2H), 0.82-0.86 (m, 2H), 0.85 (s, 9H), 1.20-1.45 (m, 2H), 1.33 (d, J = 7.40 Hz, 2H), 2.20 (td, J = 11.10, 2.70 Hz, 2H), 2.23 (t, J = 7.17 Hz, 2H), 2.29 (s, 3H), 2.40 (td, J = 8.84, 2.77 Hz, 1H), 2.54 (t, J = 8.21 Hz, 1H), 2.92 (t, | 473 | 471 | Mixture of two stereoi- somers (Stereo- chemistry on Cyclobu- tane ring: |
| | | J = 6.82 Hz, 3H), 3.06 (t, J = 7.28 Hz, | | | Mixture of |
| | | 2H), 3.56 (dd, J = 12.37, 7.05 Hz, | | | Cis-isomer |
| | | 2H), 7.06 (d, J = 7.86 Hz, 1H), 7.17 | | | and Trans- |
| | | (s, 1H), 7.88 (brs, 1H), 8.21 (d, | | | isomer) |
| | | J = 7.86 Hz, 1H) | | | |
| F-743 | | (400 MHz, DMSO-D6) 0.49 (dd, J = 9.48, 5.55 Hz, 2H), 0.78-0.83 (m, 2H), 0.84 (s, 9H), 1.32 (d, J = 6.70 Hz, 2H), 1.45-1.52 (m, 1H), 2.15 (t, J = 10.63 Hz, 2H), 2.28 (s, 3H), 2.42 (t, J = 10.17 Hz, 2H), 2.51-2.60 (m, 1H), 2.77 (t, J = 7.63 Hz, 2H), 2.88 (dd, J = 13.52, | 487 | 485 | Mixture of two stereoi- somers (Stereo- chemistry, on Cyclobu- tane ring: |
| | | 5.20 Hz, 2H), 2.96 (s, 2H), 7.12 (d, | | | Mixture of |
| | | J = 8.32 Hz, 1H), 7.31 (s, 1H), 7.55 | | | Cis-isomer |
| | | (d, J = 8.09 Hz, 1H), 9.48 (s, 1H), | | | and Trans- |
| | | 12.03 (brs, 1H) | | | isomer) |

TABLE 359

| | | | | | Informa- |
|---|---|---|---|---|---|
| | | | MS | | tion of |
| Example | Chemical structure | NMR | M + H | M − H | structure |
| F-744 | | (400 MHz, CDCl3) 0.52-0.65 (m, 2H), 0.83-0.93 (m, 2H), 1.28 (s, 9H), 1.42-1.51 (m, 1H), 2.28 (s, 3H), 2.93 (t, J = 7.17 Hz, 2H), 3.09- 3.14 (m, 2H), 3.73 (brs, 1H), 6.13 (d, J = 5.29 Hz, 1H), 6.26 (s, 1H), 7.03-7.08 (m, 1H), 7.16 (s, 1H), 7.82 (s, 1H), 8.14 (d, J = 8.38 Hz, | 458 | 456 | Racemic form |
| | | 1H) | | | |
| F-745 | | (400 MHz, CDCl3) 0.65-0.66 (m, 2H), 0.87-0.92 (m, 2H), 1.36 (d, J = 0.69 Hz, 9H), 1.52-1.57 (m, 2H), 2.28 (s, 3H), 2.95 (t, J = 7.28 Hz, 2H), 3.14 (t, J = 7.28 Hz, 2H), 6.23 (s, 1H), 7.05 (d, J = 8.55 Hz, 1H), 7.16 (s, 1H), 7.73 (s, 1H), 8.17 (d, J = 8.55 Hz, 1H) | 478 | 476 | |
| F-746 | | (400 MHz, DMSO-D6) 0.31-0.54 (m, 2H), 0.74-0.83 (m, 2H), 1.01- 1.10 (m, 6H), 1.33-1.46 (m, 1H), 1.57-1.68 (m, 2H), 1.80-1.98 (m, 4H), 2.11-2.39 (m, 5H), 2.74- 2.94 (m, 2H), 3.15-3.58 (m, 3H), 7.68-7.82 (m, 2H), 7.88 (s, 1H), 9.54 (s, 1H) | 561 | 559 | |

Formulation examples of the present invention include for example the following, but which should not be construed as limitative.

### Formulation Example 1

### Preparation of Capsule

| | | | |
|---|---|---|---|
| (1) | Compound of Example E-01 | 30 | mg |
| (2) | Microcrystalline cellulose | 10 | mg |
| (3) | Lactose | 19 | mg |
| (4) | Magnesium stearate | 1 | mg |

(1), (2), (3) and (4) are mixed and filled in a gelatin capsule

### Formulation Example 2

### Preparation of Tablet

| | | |
|---|---|---|
| (1) | Compound of Example E-01 | 10 g |
| (2) | Lactose | 50 g |
| (3) | Corn starch | 15 g |
| (4) | Carmellose calcium | 44 g |
| (5) | Magnesium stearate | 1 g |

The entire amounts of (1), (2) and (3) and 30 g of (4) are mixed with water and dried in vacuo and then granulated. The granulated powder is mixed with 14 g of (4) and 1 g of (5) and tableted by a tableting machine. In this way, 1000 tablets can be obtained, each of which contains 10 mg of Compound of Example E-01.

Formulation examples of the present invention include for example the following, but which should not be construed as limitative.

### Formulation Example 3

### Preparation of Capsule

| | | | |
|---|---|---|---|
| (1) | Compound of Example F-499 | 30 | mg |
| (2) | Microcrystalline cellulose | 10 | mg |
| (3) | Lactose | 19 | mg |
| (4) | Magnesium stearate | 1 | mg |

(1), (2), (3) and (4) are mixed and filled in a gelatin capsule.

### Formulation Example 4

### Preparation of Tablet

| | | | |
|---|---|---|---|
| (1) | Compound of Example F-499 | 10 | g |
| (2) | Lactose | 50 | g |
| (3) | Corn starch | 15 | g |
| (4) | Carmellose calcium | 44 | g |
| (5) | Magnesium stearate | 1 | g |

The entire amounts of (1), (2) and (3) and 30 g of (4) are mixed with water and dried in vacuo and then granulated. The granulated powder is mixed with 14 g of (4) and 1 g of (5) and tableted by a tableting machine. In this way, 1000 tablets can be obtained, each of which contains 10 mg of Compound of Example F-499.

### Biological Assay1

Pharmacological effects of the typical compounds of the present invention were observed.

In vitro assay of inhibitory effect against RORγ transcriptional activity

Inhibitory effect of test article on transcriptional activity of RORγ was measured by means of the following luciferase reporter gene assay.

A cDNA encoding human and mouse RORγ ligand binding domain (LBD) were obtained based on the reported sequences (Genebank accession number and sequence: human, NM_005060.3 and from Ser253 to Lys518; mouse, NM_011281.2 and from Ile251 to Lys516).

The cDNA of human RORγ or mouse RORγ was inserted into pFA-CMV vector (Strategene), which expresses GAL4-DNA binding domain fusion protein.

The resulting plasmids are hereinafter referred to as GAL4-hRORγ plasmid and GAL4-mRORγ plasmid, respectively.

Human or mouse GAL4-RORγ plasmid was transiently co-transfected into Chinese hamster ovary cells (CHO cells) with pGL5-Luc plasmid, a reporter plasmid expressing firefly luciferase depending on GAL4.

TransIT CHO transfection reagent (Mirus) was used to co-transfect human or mouse GAL4-RORγ plasmid into CHO cells with pG5-Luc plasmid.

One day before the assay, CHO cells were suspended in HAM F-12 Nutrient medium containing 10 v/v % fetal bovine serum and seeded at 6×10⁶ cells per 175 cm² cell culture flask.

Fifty four micro litters of Transit-CHO reagent was added into a 15 ml tube containing 1.16 ml of HAM F-12 Nutrient medium without fetal bovine serum and incubated at room temperature for 10 min.

A total 36 uL plasmid solution containing the GAL4-hRORγ plasmid (400 ng), pG5-Luc plasmid (9000 ng) and pcDNA3 plasmid (8600 ng) were added into the tube and mixed gently.

In case of mouse assay, the GAL4-mRORγ plasmid (250 ng), pG5-Luc plasmid (9000 ng) and pcDNA3 plasmid (8750 ng) were added.

The mixture was incubated at room temperature for 10 min.

Nine micro litters of CHO Mojo Reagent was then added into each tube and mixed gently. The mixture was incubated at room temperature for 10 min.

The resultant transfection reagent was applied to the cell culture.

After incubation at 37° C., 5% CO₂ for 4 hr, the transfected CHO cells were harvested by a trypsin treatment.

The collected cells were resuspended in HAM F-12 Nutrient medium supplemented with 10 v/v % fetal bovine serum and plated into a 384-well-white plate at 8,000 cells/50 uL/well.

The plate was incubated at room temperature for 1 hour and then further incubated at 37° C., 5% CO₂ for 3 hours.

The test articles were dissolved in dimethylsulfoxide (DMSO) to obtain a concentration of 10 mmol/L. The resulting solution was diluted with the medium just before use and added to the cells in the plate to prepare 8 different concentrations of the test article.

The final concentration of DMSO was 0.1 v/v % After the addition of the test articles, the cells were incubated at 37° C., 5% CO₂ for 2 days.

Cell viability was tested by a fluorescence method using Resazurin (invitrogen).

Two days after the addition of the test article, Resazurin was diluted with culture medium to make the 20 umol/L resazurin solution.

10 uL of the diluted resazurin solution was added into the 384-well-plate.

Then, the fluorescence was measured immediately at 615 nm with the excitation wavelength of 570 nm (0 hr reading). After incubation at 37° C., 5% CO₂ for 2 hr, the fluorescence was measured at 615 nm with the excitation wavelength of 570 nm again (2 hr reading).

The fluorescence counts (2 hr-0 hr) were calculated by subtracting the 0 hr readings from the 2 hr readings.

The luminescence count in the cells treated with 0.1% DMSO alone was defined as 100%, and the cell viability in the test article was calculated as a percentage (%-of-control) based on the value of 0.1% DMSO alone.

When the cell viability is 70% or less, it was judged that the test article has cytotoxicity.

RORγ transcriptional activity was detected as the intracellular luciferase activity using SteadyLite HTS Reporter Gene Assay System (Perkin Elmer).

StedyLite Reagent was diluted five-fold into a solution containing 10 mM Tricine, 0.2% w/v BSA, 0.02% v/v Tween-20 to obtain the luciferase substrate solution.

After the measurement of the cell viability using Resazurin, the culture media in the 384 well-plate were removed. Then the Luc substrate solution was added into each well.

After the incubation at room temperature for 10 minutes, luminescence of each well was measured by a microplate reader.

The luciferase activity derived from the luminescence count in the vehicle-control well treated with 0.1% DMSO alone was defined as 100%, and the luciferase activity in the test article was calculated as a percentage (%-of-control) based on the value of the vehicle-control.

EC50 value of test article was calculated by curve fitting with GraphPad Prism.

The luminescence counts at the concentration of the test article where the cytotoxicity was observed were excluded from the data analysis.

The results are shown in Tables 360-385.

In the tables, “+” means EC₅₀≧3 μM, and “++” means EC₅₀<3 μM.

TABLE 360

LUC EC50 (μM)

| Example | hRORg | mRORg |
|---|---|---|
| E-01 | ++ | ++ |
| E-02 | ++ | ++ |
| E-03 | ++ | ++ |
| E-04 | ++ | ++ |
| E-05 | ++ | ++ |
| E-06 | ++ | ++ |
| E-07 | + | + |
| E-08 | + | + |
| E-09 | ++ | ++ |
| E-10 | ++ | ++ |
| E-11 | ++ | ++ |
| E-12 | + | ++ |
| E-13 | + | ++ |
| E-14 | ++ | ++ |
| E-15 | ++ | ++ |
| E-16 | ++ | ++ |
| E-17 | ++ | ++ |
| E-18 | ++ | ++ |
| E-19 | + | + |
| E-20 | ++ | ++ |
| E-21 | ++ | ++ |
| E-22 | ++ | ++ |
| E-23 | ++ | ++ |
| E-24 | ++ | ++ |
| E-25 | + | ++ |
| E-26 | ++ | ++ |
| E-27 | ++ | ++ |
| E-28 | ++ | ++ |
| E-29 | ++ | ++ |
| E-30 | ++ | ++ |
| E-31 | ++ | ++ |
| E-32 | ++ | ++ |
| E-33 | ++ | ++ |

TABLE 361

LUC EC50 (μM)

| Example | hRORg | mRORg |
|---|---|---|
| E-34 | ++ | ++ |
| E-35 | ++ | ++ |
| E-36 | + | ++ |
| E-37 | ++ | ++ |
| E-38 | ++ | ++ |
| E-39 | ++ | ++ |
| E-40 | ++ | ++ |
| E-41 | ++ | ++ |
| E-42 | ++ | ++ |
| E-43 | ++ | ++ |
| E-44 | ++ | ++ |
| E-45 | ++ | ++ |
| E-46 | ++ | ++ |
| E-47 | ++ | ++ |
| E-48 | ++ | ++ |
| E-49 | ++ | ++ |
| E-50 | ++ | ++ |
| E-51 | ++ | ++ |
| E-52 | ++ | ++ |
| E-53 | ++ | ++ |
| E-54 | ++ | ++ |
| E-55 | ++ | ++ |
| E-56 | ++ | ++ |
| E-57 | ++ | ++ |
| E-58 | ++ | ++ |
| E-59 | ++ | ++ |
| E-60 | ++ | ++ |
| E-61 | ++ | ++ |
| E-62 | ++ | ++ |
| E-63 | ++ | ++ |
| E-64 | ++ | ++ |
| E-65 | ++ | ++ |
| E-66 | ++ | ++ |

TABLE 362

LUC EC50 (μM)

| Example | hRORg | mRORg |
|---|---|---|
| E-67 | ++ | ++ |
| E-68 | ++ | ++ |
| E-69 | ++ | ++ |
| E-70 | ++ | ++ |
| E-71 | ++ | ++ |
| E-72 | ++ | ++ |
| E-73 | ++ | ++ |
| E-74 | ++ | ++ |
| E-75 | ++ | ++ |
| E-76 | ++ | ++ |

TABLE 363

LUC EC50 (μM)

| Example | hRORg | mRORg |
|---|---|---|
| F-1 | ++ | ++ |
| F-2 | ++ | ++ |
| F-3 | + | ++ |
| F-4 | ++ | ++ |
| F-5 | + | + |
| F-6 | + | + |
| F-7 | + | ++ |
| F-8 | + | ++ |
| F-9 | + | + |
| F-10 | + | + |
| F-11 | + | + |
| F-12 | ++ | ++ |
| F-13 | ++ | ++ |
| F-14 | ++ | ++ |
| F-15 | + | + |
| F-16 | + | ++ |
| F-17 | + | ++ |
| F-18 | ++ | ++ |
| F-19 | + | ++ |
| F-20 | + | + |
| F-21 | + | + |
| F-22 | + | + |
| F-23 | + | + |
| F-24 | + | + |
| F-25 | + | ++ |
| F-26 | + | + |
| F-27 | + | ++ |
| F-28 | + | + |
| F-29 | + | + |
| F-30 | + | ++ |
| F-31 | + | + |
| F-32 | ++ | ++ |
| F-33 | ++ | ++ |

TABLE 364

LUC EC50 (μM)

| Example | hRORg | mRORg |
|---|---|---|
| F-34 | + | + |
| F-35 | ++ | ++ |
| F-36 | + | + |
| F-37 | + | ++ |
| F-38 | ++ | + |
| F-39 | ++ | ++ |
| F-40 | ++ | ++ |
| F-41 | ++ | ++ |
| F-42 | + | + |
| F-43 | + | + |
| F-44 | ++ | ++ |
| F-45 | + | + |
| F-46 | + | + |
| F-47 | + | ++ |
| F-48 | + | ++ |
| F-49 | + | + |
| F-50 | + | + |
| F-51 | + | + |
| F-52 | + | ++ |
| F-53 | + | ++ |
| F-54 | ++ | ++ |
| F-55 | + | ++ |
| F-56 | + | + |
| F-57 | + | + |
| F-58 | + | + |
| F-59 | + | + |
| F-60 | + | + |
| F-61 | + | + |
| F-62 | + | + |
| F-63 | + | + |
| F-64 | + | + |
| F-65 | + | + |
| F-66 | + | + |

TABLE 365

LUC EC50 (μM)

| Example | hRORg | mRORg |
|---|---|---|
| F-67 | + | + |
| F-68 | + | + |
| F-69 | + | ++ |
| F-70 | + | + |
| F-71 | + | + |
| F-72 | + | + |
| F-73 | + | + |
| F-74 | + | + |
| F-75 | + | + |
| F-76 | + | + |
| F-77 | + | + |
| F-78 | + | ++ |
| F-79 | + | + |
| F-80 | + | + |
| F-81 | ++ | ++ |
| F-82 | + | + |
| F-83 | + | + |
| F-84 | ++ | ++ |
| F-85 | + | + |
| F-86 | + | + |
| F-87 | + | + |
| F-88 | + | + |
| F-89 | + | + |
| F-90 | + | + |
| F-91 | + | + |
| F-92 | + | + |
| F-93 | + | + |
| F-94 | ++ | ++ |
| F-95 | ++ | ++ |
| F-96 | + | + |
| F-97 | + | + |
| F-98 | + | ++ |
| F-99 | + | ++ |

TABLE 366

LUC EC50 (μM)

| Example | hRORg | mRORg |
|---|---|---|
| F-100 | + | + |
| F-101 | + | + |
| F-102 | + | + |
| F-103 | + | + |
| F-104 | + | + |
| F-105 | + | + |
| F-106 | + | + |
| F-107 | + | + |
| F-108 | + | + |
| F-109 | + | + |
| F-110 | + | + |
| F-111 | + | + |
| F-112 | + | ++ |
| F-113 | + | + |
| F-114 | + | + |
| F-115 | + | + |
| F-116 | + | + |
| F-117 | + | ++ |
| F-118 | + | + |
| F-119 | ++ | ++ |
| F-120 | ++ | ++ |
| F-121 | + | + |
| F-122 | + | + |
| F-123 | + | + |
| F-124 | + | + |
| F-125 | + | + |
| F-126 | + | + |
| F-127 | + | + |
| F-128 | + | + |
| F-129 | + | + |
| F-130 | + | + |
| F-131 | + | + |
| F-132 | + | + |

TABLE 367

LUC EC50 (μM)

| Example | hRORg | mRORg |
|---|---|---|
| F-133 | + | + |
| F-134 | + | + |
| F-135 | + | ++ |
| F-136 | + | + |
| F-137 | + | ++ |
| F-138 | + | ++ |
| F-139 | + | + |
| F-140 | + | + |
| F-141 | + | ++ |
| F-142 | ++ | ++ |
| F-143 | + | + |
| F-144 | ++ | ++ |
| F-145 | ++ | ++ |
| F-146 | + | ++ |
| F-147 | + | ++ |
| F-148 | ++ | ++ |
| F-149 | + | + |
| F-150 | + | + |
| F-151 | + | + |
| F-152 | + | + |
| F-153 | + | + |
| F-154 | + | + |
| F-155 | ++ | ++ |
| F-156 | + | ++ |
| F-157 | + | + |
| F-158 | + | ++ |
| F-159 | + | + |
| F-160 | ++ | ++ |
| F-161 | + | + |
| F-162 | + | ++ |
| F-163 | ++ | ++ |
| F-164 | + | ++ |
| F-165 | + | ++ |

TABLE 368

LUC EC50 (μM)

| Example | hRORg | mRORg |
|---|---|---|
| F-166 | + | ++ |
| F-167 | ++ | ++ |
| F-168 | + | ++ |
| F-169 | + | + |
| F-170 | + | + |
| F-171 | + | + |
| F-172 | + | + |
| F-173 | ++ | ++ |
| F-174 | + | + |
| F-175 | ++ | ++ |
| F-176 | + | + |
| F-177 | ++ | ++ |
| F-178 | ++ | ++ |
| F-179 | + | + |
| F-180 | + | ++ |
| F-181 | ++ | ++ |
| F-182 | + | ++ |
| F-183 | + | + |
| F-184 | + | + |
| F-185 | + | + |
| F-186 | ++ | ++ |
| F-187 | + | + |
| F-188 | ++ | ++ |
| F-189 | + | + |
| F-190 | + | + |
| F-191 | ++ | ++ |
| F-192 | + | + |
| F-193 | ++ | ++ |
| F-194 | ++ | ++ |
| F-195 | ++ | ++ |
| F-196 | ++ | ++ |
| F-197 | ++ | ++ |
| F-198 | ++ | ++ |

TABLE 369

LUC EC50 (μM)

| Example | hRORg | mRORg |
|---|---|---|
| F-199 | ++ | ++ |
| F-200 | ++ | ++ |
| F-201 | ++ | ++ |
| F-202 | ++ | ++ |
| F-203 | + | + |
| F-204 | + | + |
| F-205 | + | + |
| F-206 | ++ | ++ |
| F-207 | ++ | ++ |
| F-208 | + | + |
| F-209 | + | + |
| F-210 | ++ | ++ |
| F-211 | ++ | ++ |
| F-212 | ++ | ++ |
| F-213 | + | + |
| F-214 | ++ | ++ |
| F-215 | ++ | ++ |
| F-216 | ++ | ++ |
| F-217 | ++ | ++ |
| F-218 | + | ++ |
| F-219 | ++ | ++ |
| F-220 | ++ | ++ |
| F-221 | + | + |
| F-222 | + | ++ |
| F-223 | ++ | ++ |
| F-224 | ++ | ++ |
| F-225 | + | + |
| F-226 | + | + |
| F-227 | ++ | ++ |
| F-228 | ++ | ++ |
| F-229 | + | + |
| F-230 | ++ | ++ |
| F-231 | + | + |

TABLE 370

LUC EC50 (μM)

| Example | hRORg | mRORg |
|---|---|---|
| F-232 | + | + |
| F-233 | + | + |
| F-234 | + | ++ |
| F-235 | ++ | ++ |
| F-236 | + | ++ |
| F-237 | + | + |
| F-238 | + | + |
| F-239 | + | + |
| F-240 | + | ++ |
| F-241 | ++ | ++ |
| F-242 | + | ++ |
| F-243 | ++ | + |
| F-244 | ++ | ++ |
| F-245 | ++ | ++ |
| F-246 | ++ | ++ |
| F-247 | ++ | ++ |
| F-248 | ++ | ++ |
| F-249 | ++ | ++ |
| F-250 | + | + |
| F-251 | ++ | ++ |
| F-252 | + | + |
| F-253 | + | + |
| F-254 | ++ | ++ |
| F-255 | + | + |
| F-256 | ++ | ++ |
| F-257 | ++ | ++ |
| F-258 | + | + |
| F-259 | + | + |
| F-260 | + | + |
| F-261 | + | ++ |
| F-262 | + | + |
| F-263 | ++ | ++ |
| F-264 | + | + |

TABLE 371

| | LUC EC50 (μM) | |
|---|---|---|
| Example | hRORg | mRORg |
| F-265 | + | ++ |
| F-266 | ++ | ++ |
| F-267 | ++ | ++ |
| F-268 | ++ | ++ |
| F-269 | ++ | ++ |
| F-270 | ++ | ++ |
| F-271 | ++ | ++ |
| F-272 | ++ | ++ |
| F-273 | + | + |
| F-274 | + | + |
| F-275 | + | + |
| F-276 | + | + |
| F-277 | + | ++ |
| F-278 | + | ++ |
| F-279 | ++ | ++ |
| F-280 | ++ | ++ |
| F-281 | ++ | ++ |
| F-282 | + | + |
| F-283 | + | + |
| F-284 | + | + |
| F-285 | + | ++ |
| F-286 | ++ | ++ |
| F-287 | + | + |
| F-288 | + | + |
| F-289 | ++ | ++ |
| F-290 | + | + |
| F-291 | ++ | ++ |
| F-292 | + | ++ |
| F-293 | ++ | ++ |
| F-294 | + | + |
| F-295 | ++ | ++ |
| F-296 | ++ | ++ |
| F-297 | ++ | ++ |

TABLE 372

| | LUC EC50 (μM) | |
|---|---|---|
| Example | hRORg | mRORg |
| F-298 | ++ | ++ |
| F-299 | ++ | ++ |
| F-300 | ++ | ++ |
| F-301 | ++ | ++ |
| F-302 | ++ | ++ |
| F-303 | ++ | ++ |
| F-304 | ++ | ++ |
| F-305 | ++ | ++ |
| F-306 | ++ | ++ |
| F-307 | ++ | ++ |
| F-308 | ++ | ++ |
| F-309 | ++ | ++ |
| F-310 | ++ | ++ |
| F-311 | ++ | ++ |
| F-312 | + | + |
| F-313 | + | + |
| F-314 | ++ | ++ |
| F-315 | ++ | ++ |
| F-316 | + | + |
| F-317 | ++ | ++ |
| F-318 | + | + |
| F-319 | ++ | ++ |
| F-320 | ++ | ++ |
| F-321 | ++ | ++ |
| F-322 | ++ | ++ |
| F-323 | ++ | ++ |
| F-324 | ++ | ++ |
| F-325 | ++ | ++ |
| F-326 | + | + |
| F-327 | ++ | ++ |
| F-328 | ++ | ++ |
| F-329 | + | + |
| F-330 | + | + |

TABLE 373

| | LUC EC50 (μM) | |
|---|---|---|
| Example | hRORg | mRORg |
| F-331 | ++ | ++ |
| F-332 | ++ | ++ |
| F-333 | ++ | ++ |
| F-334 | ++ | ++ |
| F-335 | + | + |
| F-336 | + | + |
| F-337 | + | + |
| F-338 | ++ | ++ |
| F-339 | + | + |
| F-340 | + | ++ |
| F-341 | ++ | ++ |
| F-342 | + | + |
| F-343 | ++ | ++ |
| F-344 | ++ | ++ |
| F-345 | ++ | ++ |
| F-346 | ++ | ++ |
| F-347 | ++ | ++ |
| F-348 | + | + |
| F-349 | + | + |
| F-350 | ++ | ++ |
| F-351 | ++ | ++ |
| F-352 | + | + |
| F-353 | ++ | ++ |
| F-354 | ++ | ++ |
| F-355 | ++ | ++ |
| F-356 | ++ | ++ |
| F-357 | ++ | ++ |
| F-358 | ++ | ++ |
| F-359 | ++ | ++ |
| F-360 | + | + |
| F-361 | ++ | ++ |
| F-362 | + | + |
| F-363 | ++ | ++ |

TABLE 374

| | LUC EC50 (μM) | |
|---|---|---|
| Example | hRORg | mRORg |
| F-364 | ++ | ++ |
| F-365 | + | ++ |
| F-366 | ++ | ++ |
| F-367 | + | ++ |
| F-368 | + | + |
| F-369 | ++ | ++ |
| F-370 | ++ | ++ |
| F-371 | ++ | ++ |
| F-372 | + | + |
| F-373 | ++ | ++ |
| F-374 | ++ | ++ |
| F-375 | + | + |
| F-376 | ++ | ++ |
| F-377 | undetermined | undetermined |
| F-378 | undetermined | undetermined |
| F-379 | + | ++ |
| F-380 | ++ | ++ |
| F-381 | + | ++ |
| F-382 | ++ | ++ |
| F-383 | ++ | ++ |
| F-384 | + | + |
| F-385 | ++ | ++ |
| F-386 | + | + |
| F-387 | ++ | ++ |
| F-388 | ++ | ++ |
| F-389 | ++ | ++ |
| F-390 | + | + |
| F-391 | ++ | ++ |
| F-392 | ++ | ++ |
| F-393 | + | + |
| F-394 | ++ | ++ |
| F-395 | ++ | ++ |
| F-396 | ++ | ++ |

TABLE 375

| | LUC EC50 (μM) | |
|---|---|---|
| Example | hRORg | mRORg |
| F-397 | ++ | ++ |
| F-398 | ++ | ++ |
| F-399 | ++ | ++ |
| F-400 | ++ | ++ |
| F-401 | ++ | ++ |
| F-402 | ++ | ++ |
| F-403 | ++ | ++ |
| F-404 | ++ | ++ |
| F-405 | ++ | ++ |
| F-406 | ++ | ++ |
| F-407 | + | + |
| F-408 | ++ | ++ |
| F-409 | + | + |
| F-410 | ++ | ++ |
| F-411 | ++ | ++ |
| F-412 | ++ | ++ |
| F-413 | ++ | ++ |
| F-414 | ++ | ++ |
| F-415 | + | ++ |
| F-416 | + | + |
| F-417 | + | + |
| F-418 | + | + |
| F-419 | + | + |
| F-420 | ++ | ++ |
| F-421 | ++ | ++ |
| F-422 | ++ | ++ |
| F-423 | ++ | ++ |
| F-424 | ++ | ++ |
| F-425 | ++ | ++ |
| F-426 | ++ | ++ |
| F-427 | + | ++ |
| F-428 | ++ | ++ |
| F-429 | ++ | ++ |

TABLE 376

| | LUC EC50 (μM) | |
|---|---|---|
| Example | hRORg | mRORg |
| F-430 | ++ | ++ |
| F-431 | ++ | ++ |
| F-432 | ++ | ++ |
| F-433 | ++ | ++ |
| F-434 | ++ | ++ |
| F-435 | ++ | ++ |
| F-436 | ++ | ++ |
| F-437 | ++ | ++ |
| F-438 | ++ | ++ |
| F-439 | ++ | ++ |
| F-440 | ++ | ++ |
| F-441 | + | ++ |
| F-442 | + | + |
| F-443 | ++ | ++ |
| F-444 | ++ | ++ |
| F-445 | ++ | ++ |
| F-446 | ++ | ++ |
| F-447 | ++ | ++ |
| F-448 | ++ | ++ |
| F-449 | + | + |
| F-450 | ++ | ++ |
| F-451 | ++ | ++ |
| F-452 | ++ | ++ |
| F-453 | ++ | ++ |
| F-454 | ++ | ++ |
| F-455 | + | ++ |
| F-456 | ++ | ++ |
| F-457 | ++ | + |
| F-458 | + | + |
| F-459 | ++ | ++ |
| F-460 | + | + |
| F-461 | ++ | ++ |
| F-462 | ++ | ++ |

TABLE 377

| | LUC EC50 (μM) | |
|---|---|---|
| Example | hRORg | mRORg |
| F-463 | + | ++ |
| F-464 | + | ++ |
| F-465 | ++ | ++ |
| F-466 | ++ | ++ |
| F-467 | + | + |
| F-468 | + | + |
| F-469 | + | + |
| F-470 | ++ | ++ |
| F-471 | + | + |
| F-472 | ++ | ++ |
| F-473 | ++ | ++ |
| F-474 | + | ++ |
| F-475 | ++ | ++ |
| F-476 | ++ | ++ |
| F-477 | ++ | ++ |
| F-478 | + | ++ |
| F-479 | ++ | ++ |
| F-480 | ++ | ++ |
| F-481 | + | + |
| F-482 | ++ | ++ |
| F-483 | ++ | ++ |
| F-484 | ++ | ++ |
| F-485 | ++ | ++ |
| F-486 | ++ | ++ |
| F-487 | ++ | ++ |
| F-488 | + | ++ |
| F-489 | + | ++ |
| F-490 | + | + |
| F-491 | + | ++ |
| F-492 | + | + |
| F-493 | ++ | ++ |
| F-494 | ++ | ++ |
| F-495 | ++ | ++ |

TABLE 378

| | LUC EC50 (μM) | |
|---|---|---|
| Example | hRORg | mRORg |
| F-496 | ++ | ++ |
| F-497 | ++ | ++ |
| F-498 | ++ | ++ |
| F-499 | + | ++ |
| F-500 | ++ | ++ |
| F-501 | ++ | ++ |
| F-502 | + | + |
| F-503 | + | + |
| F-504 | ++ | ++ |
| F-505 | ++ | + |
| F-506 | ++ | ++ |
| F-507 | + | + |
| F-508 | + | + |
| F-509 | ++ | ++ |
| F-510 | + | + |
| F-511 | ++ | ++ |
| F-512 | ++ | ++ |
| F-513 | ++ | ++ |
| F-514 | + | + |
| F-515 | ++ | ++ |
| F-516 | ++ | ++ |
| F-517 | ++ | ++ |
| F-518 | ++ | ++ |
| F-519 | ++ | ++ |
| F-520 | ++ | ++ |
| F-521 | ++ | ++ |
| F-522 | + | + |
| F-523 | ++ | ++ |
| F-524 | ++ | ++ |
| F-525 | ++ | ++ |
| F-526 | ++ | ++ |
| F-527 | ++ | ++ |
| F-528 | ++ | ++ |

TABLE 379

| | LUC EC50 (μM) | |
|---|---|---|
| Example | hRORg | mRORg |
| F-529 | ++ | ++ |
| F-530 | ++ | ++ |
| F-531 | ++ | ++ |
| F-532 | + | + |
| F-533 | ++ | ++ |
| F-534 | ++ | ++ |
| F-535 | ++ | ++ |
| F-536 | ++ | ++ |
| F-537 | ++ | ++ |
| F-538 | ++ | ++ |
| F-539 | + | ++ |
| F-540 | ++ | ++ |
| F-541 | ++ | ++ |
| F-542 | ++ | ++ |
| F-543 | ++ | ++ |
| F-544 | ++ | ++ |
| F-545 | ++ | ++ |
| F-546 | ++ | ++ |
| F-547 | ++ | ++ |
| F-548 | ++ | ++ |
| F-549 | ++ | ++ |
| F-550 | ++ | ++ |
| F-551 | ++ | ++ |
| F-552 | ++ | ++ |
| F-553 | ++ | ++ |
| F-554 | ++ | ++ |
| F-555 | + | + |
| F-556 | ++ | ++ |
| F-557 | + | + |
| F-558 | ++ | ++ |
| F-559 | ++ | ++ |
| F-560 | ++ | ++ |
| F-561 | ++ | ++ |

TABLE 380

| | LUC EC50 (μM) | |
|---|---|---|
| Example | hRORg | mRORg |
| F-562 | ++ | ++ |
| F-563 | ++ | ++ |
| F-564 | ++ | ++ |
| F-565 | ++ | ++ |
| F-566 | ++ | ++ |
| F-567 | ++ | ++ |
| F-568 | ++ | ++ |
| F-569 | ++ | ++ |
| F-570 | ++ | + |
| F-571 | ++ | ++ |
| F-572 | ++ | ++ |
| F-573 | ++ | ++ |
| F-574 | ++ | ++ |
| F-575 | ++ | ++ |
| F-576 | ++ | ++ |
| F-577 | ++ | ++ |
| F-578 | ++ | ++ |
| F-579 | ++ | ++ |
| F-580 | ++ | ++ |
| F-581 | ++ | ++ |
| F-582 | ++ | ++ |
| F-583 | ++ | ++ |
| F-584 | + | ++ |
| F-585 | ++ | ++ |
| F-586 | ++ | ++ |
| F-587 | ++ | ++ |
| F-588 | + | ++ |
| F-589 | + | + |
| F-590 | ++ | ++ |
| F-591 | ++ | ++ |
| F-592 | ++ | ++ |
| F-593 | + | ++ |
| F-594 | ++ | ++ |

TABLE 381

| | LUC EC50 (μM) | |
|---|---|---|
| Example | hRORg | mRORg |
| F-595 | ++ | ++ |
| F-596 | ++ | ++ |
| F-597 | ++ | ++ |
| F-598 | + | + |
| F-599 | ++ | ++ |
| F-600 | ++ | ++ |
| F-601 | ++ | ++ |
| F-602 | ++ | ++ |
| F-603 | ++ | ++ |
| F-604 | ++ | ++ |
| F-605 | ++ | ++ |
| F-606 | ++ | ++ |
| F-607 | ++ | ++ |
| F-608 | ++ | ++ |
| F-609 | ++ | ++ |
| F-610 | ++ | ++ |
| F-611 | ++ | ++ |
| F-612 | ++ | ++ |
| F-613 | ++ | ++ |
| F-614 | ++ | ++ |
| F-615 | ++ | ++ |
| F-616 | ++ | ++ |
| F-617 | ++ | ++ |
| F-618 | ++ | ++ |
| F-619 | ++ | ++ |
| F-620 | ++ | ++ |
| F-621 | ++ | ++ |
| F-622 | ++ | ++ |
| F-623 | ++ | ++ |
| F-624 | ++ | ++ |
| F-625 | ++ | ++ |
| F-626 | ++ | ++ |
| F-627 | ++ | ++ |

TABLE 382

| | LUC EC50 (μM) | |
|---|---|---|
| Example | hRORg | mRORg |
| F-628 | ++ | ++ |
| F-629 | ++ | ++ |
| F-630 | ++ | ++ |
| F-631 | ++ | ++ |
| F-632 | ++ | ++ |
| F-633 | ++ | ++ |
| F-634 | ++ | ++ |
| F-635 | ++ | ++ |
| F-636 | ++ | ++ |
| F-637 | ++ | ++ |
| F-638 | ++ | ++ |
| F-639 | ++ | ++ |
| F-640 | ++ | ++ |
| F-641 | ++ | ++ |
| F-642 | ++ | ++ |
| F-643 | ++ | ++ |
| F-644 | ++ | ++ |
| F-645 | + | + |
| F-646 | + | + |
| F-647 | ++ | ++ |
| F-648 | ++ | ++ |
| F-649 | ++ | ++ |
| F-650 | ++ | ++ |
| F-651 | ++ | ++ |
| F-652 | ++ | ++ |
| F-653 | ++ | ++ |
| F-654 | + | ++ |
| F-655 | ++ | ++ |
| F-656 | ++ | ++ |
| F-657 | + | ++ |
| F-658 | ++ | ++ |
| F-659 | ++ | ++ |
| F-660 | ++ | ++ |

TABLE 383

| | LUC EC50 (μM) | |
|---|---|---|
| Example | hRORg | mRORg |
| F-661 | ++ | ++ |
| F-662 | ++ | ++ |
| F-663 | + | + |
| F-664 | + | + |
| F-665 | ++ | ++ |
| F-666 | + | ++ |
| F-667 | + | ++ |
| F-668 | + | ++ |
| F-669 | + | ++ |
| F-670 | + | ++ |
| F-671 | ++ | ++ |
| F-672 | ++ | ++ |
| F-673 | ++ | ++ |
| F-674 | ++ | ++ |
| F-675 | ++ | ++ |
| F-676 | ++ | ++ |
| F-677 | ++ | ++ |
| F-678 | ++ | undetermined |
| F-679 | ++ | ++ |
| F-680 | ++ | ++ |
| F-681 | ++ | ++ |
| F-682 | ++ | ++ |
| F-683 | ++ | ++ |
| F-684 | ++ | ++ |
| F-685 | ++ | ++ |
| F-686 | ++ | ++ |
| F-687 | ++ | ++ |
| F-688 | ++ | ++ |
| F-689 | ++ | ++ |
| F-690 | + | ++ |
| F-691 | ++ | ++ |
| F-692 | ++ | ++ |
| F-693 | ++ | ++ |

TABLE 384

| | LUC EC50 (μM) | |
|---|---|---|
| Example | hRORg | mRORg |
| F-694 | ++ | ++ |
| F-695 | + | ++ |
| F-696 | ++ | ++ |
| F-697 | ++ | ++ |
| F-698 | ++ | ++ |
| F-699 | ++ | ++ |
| F-700 | ++ | ++ |
| F-701 | ++ | ++ |
| F-702 | ++ | ++ |
| F-703 | ++ | ++ |
| F-704 | + | + |
| F-705 | + | + |
| F-706 | ++ | ++ |
| F-707 | ++ | ++ |
| F-708 | ++ | ++ |
| F-709 | ++ | ++ |
| F-710 | ++ | ++ |
| F-711 | + | + |
| F-712 | + | + |
| F-713 | ++ | ++ |
| F-714 | ++ | ++ |
| E-77 | ++ | ++ |
| E-78 | ++ | ++ |
| E-79 | ++ | ++ |
| E-80 | ++ | ++ |
| F-715 | ++ | ++ |
| F-716 | ++ | ++ |
| F-717 | ++ | ++ |
| F-718 | ++ | ++ |
| F-719 | ++ | ++ |
| F-720 | ++ | ++ |
| F-721 | ++ | ++ |
| F-722 | + | + |

TABLE 385

| | LUC EC50 (μM) | |
|---|---|---|
| | hRORg | mRORg |
| F-723 | + | + |
| F-724 | ++ | ++ |
| F-725 | ++ | ++ |
| F-726 | + | + |
| F-727 | + | + |
| F-728 | ++ | ++ |
| F-729 | ++ | ++ |
| F-730 | ++ | ++ |
| F-731 | + | + |
| F-732 | ++ | ++ |
| F-733 | ++ | ++ |
| F-734 | ++ | ++ |
| F-735 | ++ | ++ |
| F-736 | + | + |
| F-737 | + | + |
| F-738 | + | + |
| F-739 | + | + |
| F-740 | ++ | ++ |
| F-741 | ++ | ++ |
| F-742 | + | + |
| F-743 | + | + |
| F-744 | + | ++ |
| F-745 | ++ | ++ |
| F-746 | + | + |

1. A compound represented by Formula [I]:
or a pharmaceutically acceptable salt thereof, wherein
is
is monocyclic heteroaromatic group wherein the monocyclic heteroaromatic ring comprises the same or different 1 to 4 hetero atoms selected from nitrogen atom, oxygen atom, or sulfur atom besides carbon atom;
each R^{a1} is the same or different and selected from
(1) C₁₋₆ alkyl group which may optionally be substituted with the same or different 1 to 5 substituents selected from Group A,
(2) halogen atom, or
(3) C₃₋₇ cycloalkyl group which may optionally be substituted with the same or different 1 to 5 substituents selected from Group A;
R^{b} is
(1) C₁₋₆ alkyl group which may optionally be substituted with the same or different 1 to 5 halogen atoms, or
(2) C₃₋₇ cycloalkyl group;
R^{c} is
(1) hydrogen atom, or
(2) C₁₋₆ alkyl group;
each R^{d} is the same or different and selected from
(1) halogen atom, or
(2) C₁₋₆ alkyl group which may optionally be substituted with the same or different 1 to 5 halogen atoms;
R^{e} is hydrogen atom;
n^{a} is an integer selected from 0 or 1 to 3;
n^{c} is an integer selected from 0 or 1 to 3;
n^{d} is an integer selected from 0 or 1 to 3;
m is an integer selected from 0 or 1 to 5;
Group A is
(a) C₁₋₆ alkyl group,
(b) halogen atom,
(c) C₃₋₇ cycloalkyl group which may optionally be substituted with the same or different 1 to 5 substituents selected from the group consisting of C₁₋₆ alkyl group and halogen atom.

2. The compound according to claim 1, or a pharmaceutically acceptable salt thereof, wherein
is monocyclic heteroaromatic group selected from the following (1) to (7):

3. The compound according to claim 1, or a pharmaceutically acceptable salt thereof, wherein the compound is represented by Formula [II-A]:
wherein each symbol is as defined in claim 1.

4. The compound according to claim 1, or a pharmaceutically acceptable salt thereof, wherein the compound is represented by Formula [II-B]:
wherein each symbol is as defined in claim 1.

5. The compound according to claim 1, or a pharmaceutically acceptable salt thereof, wherein the compound is represented by Formula [II-C]:
wherein each symbol is as defined in claim 1.

6. The compound according to claim 1, or a pharmaceutically acceptable salt thereof, wherein the compound is represented by Formula [II-D]:
wherein each symbol is as defined in claim 1.

7. The compound according to claim 1, or a pharmaceutically acceptable salt thereof, wherein the compound is represented by Formula [II-E]:
wherein each symbol is as defined in claim 1.

8. The compound according to claim 1, or a pharmaceutically acceptable salt thereof, wherein the compound is represented by Formula [II-F]:
wherein each symbol is as defined in claim 1.

9. The compound according to claim 1, or a pharmaceutically acceptable salt thereof, wherein the compound is represented by Formula [II-G]:
wherein each symbol is as defined in claim 1.

10. The compound according to claim 1, or a pharmaceutically acceptable salt thereof, wherein R^{c} is hydrogen atom.

11. A pharmaceutical composition comprising the compound according to claim 1 or a pharmaceutically acceptable salt thereof, and pharmaceutically acceptable carrier.

12. A method of inhibiting RORγ in a mammal, comprising administering to the mammal a therapeutically effective amount of the compound according to claim 1 or a pharmaceutically acceptable salt thereof.

13. A method for treating or preventing a autoimmune disease, allergic disease, dry eye, fibrosis, or metabolic disease in a mammal, comprising administering to the mammal a therapeutically effective amount of the compound according to claim 1 or a pharmaceutically acceptable salt thereof.

14. The method according to claim 13, wherein the autoimmune disease is selected from the group consisting of rheumatoid arthritis, psoriasis, inflammatory bowel disease, multiple sclerosis, systemic lupus erythematosus, ankylosing spondylitis, uveitis, polymyalgia rheumatica and type I diabetes.

15. The method according to claim 13, wherein the metabolic disease is diabetes.

16. A compound represented by the following formulas:
or a pharmaceutically acceptable salt thereof, wherein
is unsaturated heteromonocyclic group selected from the following (i) to (v):
R^{a} is selected from the following (1) to (12):
(1) C₁₋₁₂ alkyl group which may optionally be substituted with the same or different 1 to 5 substituents selected from Group A,
(2) C₃₋₇ cycloalkyl group which may optionally be substituted with the same or different 1 to 5 substituents selected from Group A,
(3) C₂₋₁₂ alkenyl group which may optionally be substituted with the same or different 1 to 5 substituents selected from Group A,
(4) C₂₋₁₂ alkynyl group which may optionally be substituted with the same or different 1 to 5 substituents selected from Group A,
(5) C₅₋₁₁ spirocyclic cycloalkyl group which may optionally be substituted with the same or different 1 to 5 substituents selected from Group A,
(6) cross-linked C₅₋₁₂ cycloalkyl group which may optionally be substituted with the same or different 1 to 5 substituents selected from Group A,
(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,
(8) phenyl group which may optionally be substituted with the same or different 1 to 5 substituents selected from Group A,
(9) C₉₋₁₀ fused carbocyclic group which may optionally be substituted with the same or different 1 to 5 substituents selected from Group A,
(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,
(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;
R^{b} is selected from the following (1) to (6):
(1) C₁₋₆ alkyl group which may optionally be substituted with the same or different 1 to 5 halogen atoms,
(2) C₃₋₇ cycloalkyl group which may optionally be substituted with the same or different 1 to 5 substituents selected from the group consisting of halogen atom and C₁₋₆ alkyl group,
(3) —CH═CH—C(═O)—OR^{bb1},
(4) —CH₂—CH₂—C(═O)—OR^{bb2},
(5) —CH₂—O—CH₂—C(═O)—OR^{bb3},
(6) hydrogen atom;
R^{bb1}, R^{bb2}, and R^{bb3} are each independently hydrogen atom or C₁₋₆ alkyl group;
each R^{d} is the same or different and selected from the following (1) to (13):
(1) halogen atom,
(2) C₁₋₆ alkyl group which may optionally be substituted with the same or different 1 to 5 substituents selected from Group B,
(3) C₃₋₇ cycloalkyl group which may optionally be substituted with the same or different 1 to 5 substituents selected from Group B,
(4) C₂₋₆ alkenyl group,
(5) cyano group,
(6) —C(═O)—OR^{dd1},
(7) —C(═O)—NR^{dd2}R^{dd3},
(8) —OR^{dd4},
(9) —NR^{dd5}—C(═O)—R^{dd6},
(10) —NR^{dd7}—C(═O)—NR^{dd8}R^{dd9},
(11) —NR^{dd10}—S(═O)₂—R^{dd11},
(12) —NR^{dd12}—S(═O)₂—NR^{dd13}R^{dd14},
(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
R^{c} is hydrogen atom or C₁₋₆ alkyl group;
each R^{j} is the same or different C₁₋₁₂ alkyl group which may optionally be substituted with the same or different 1 to 5 substituents selected from Group A;
Q is selected from the following (1) to (9):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,
(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;
Y is selected from the following (1) to (3):
(1) single bond,
(2) —S(═O)₂—,
(3) C₁₋₃ alkylene which may optionally be substituted with 1 to 3 hydroxyl groups;
m is each independently an integer selected from 0 or 1 to 5;
n^{j} is each independently 0, 1 or 2;
Group A consists of the following (a) to (m):
(a) C₁₋₇ alkyl group which may optionally be substituted with the same or different 1 to 5 substituents selected from Group AA,
(b) halogen atom,
(c) phenyl group,
(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,
(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(g) C₃₋₇ cycloalkyl group which may optionally be substituted with the same or different 1 to 5 substituents selected from Group AA,
(h) —C(═O)—NR^{A1}R^{A2},
(i) —C(═O)—OR^{A3},
(j) —C(═O)—R^{A4},
(k) —OR^{A5},
(l) —NR^{A6}R^{A7},
(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
hydrogen atom,
C₁₋₆ alkyl group which may optionally be substituted with the same or different 1 to 6 halogen atoms,
C₃₋₇ cycloalkyl group which may optionally be substituted with the same or different 1 to 5 substituents selected from Group AA,
benzyl group which may optionally be substituted with the same or different 1 to 5 substituents selected from Group AA,
phenyl group which may optionally be substituted with the same or different 1 to 5 substituents selected from Group AA,
C₁₋₆ alkylcarbonyl group,
C₁₋₆ alkylsulfonyl group, or
C₂₋₆ alkenyl group; and
R^{A8} is each independently C₁₋₆ alkyl group;
Group AA consists of the following (a) to (o):
(a) halogen atom,
(b) C₁₋₃ alkyl group which may optionally be substituted with the same or different 1 to 6 halogen atoms,
(c) —OR^{AA1},
(d) —C(═O)—NR^{AA2}R^{AA3},
(e) —C(═O)—OR^{AA4},
(f) —O—C(═O)—R^{AA5},
(g) —C(═O)—R^{AA6},
(h) ═O,
(i) C₃₋₇ cycloalkyl group,
(j) phenyl group which may optionally be substituted with the same or different 1 to 5 C₁₋₃ alkyl groups,
(k) —NR^{AA7}R^{AA8},
(l) —NR^{AA9}—C(═O)—R^{AA10},
(m) —NR^{AA11}—C(═O)—NR^{AA12}R^{AA13},
(n) —NR^{AA14}—S(═O)₂—R^{AA15},
(o) —NR^{AA16}—S(═O)₂—NR^{AA17}R^{AA18};
R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):
(a) halogen atom,
(b) C₃₋₇ cycloalkyl group
(c) —OR^{B1},
(d) —C(═O)—NR^{B2}R^{B3},
(e) —C(═O)—OR^{B4},
(f) C₁₋₆ alkyl group,
(g) —NR^{B5}R^{B6},
(h) —NR^{B7}—C(═O)—R^{B8},
(i) —NR^{B9}—C(═O)—NR^{B10}R^{B11},
(j) —NR^{B12}—S(═O)₂—R^{B13},
(k) —NR^{B14}—S(═O)₂—NR^{B15}R^{B16},
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently, hydrogen atom or C₁₋₆ alkyl group;
provided that when R^{a} is
(1) phenyl group which may optionally be substituted with the same or different 1 to 5 substituents selected from Group A,
(2) C₉₋₁₀ fused carbocyclic group which may optionally be substituted with the same or different 1 to 5 substituents selected from Group A, or
(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
Q is selected from the following (1) to (6):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;
and further provided that when R^{b} is hydrogen atom,
Q is selected from the following (1) to (5):
(1) phenyl group,
(2) C₃₋₇ cycloalkyl group,
(3) C₉₋₁₀ fused carbocyclic group,
(4) cross-linked C₅₋₁₂ cycloalkyl group,
(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered.

17. A compound represented by the following formulas:
or a pharmaceutically acceptable salt thereof, wherein
is unsaturated heteromonocyclic group selected from the following (i) to (v):
R^{a} is selected from the following (1) to (12):
(1) C₁₋₁₂ alkyl group which may optionally be substituted with the same or different 1 to 5 substituents selected from Group A,
(2) C₃₋₇ cycloalkyl group which may optionally be substituted with the same or different 1 to 5 substituents selected from Group A,
(3) C₂₋₁₂ alkenyl group which may optionally be substituted with the same or different 1 to 5 substituents selected from Group A,
(4) C₂₋₁₂ alkynyl group which may optionally be substituted with the same or different 1 to 5 substituents selected from Group A,
(5) C₅₋₁₁ spirocyclic cycloalkyl group which may optionally be substituted with the same or different 1 to 5 substituents selected from Group A,
(6) cross-linked C₅₋₁₂ cycloalkyl group which may optionally be substituted with the same or different 1 to 5 substituents selected from Group A,
(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,
(8) phenyl group which may optionally be substituted with the same or different 1 to 5 substituents selected from Group A,
(9) C₉₋₁₀ fused carbocyclic group which may optionally be substituted with the same or different 1 to 5 substituents selected from Group A,
(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,
(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;
R^{b} is selected from the following (1) to (6):
(1) C₁₋₆ alkyl group which may optionally be substituted with the same or different 1 to 5 halogen atoms,
(2) C₃₋₇ cycloalkyl group which may optionally be substituted with the same or different 1 to 5 substituents selected from the group consisting of halogen atom and C₁₋₆ alkyl group,
(3) —CH═CH—C(═O)—OR^{bb1},
(4) —CH₂—CH₂—C(═O)—OR^{bb2},
(5) —CH₂—O—CH₂—C(═O)—OR^{bb3},
(6) hydrogen atom;
R^{bb1}, R^{bb2}, and R^{bb3} are each independently hydrogen atom or C₁₋₆ alkyl group;
each R^{d} is the same or different and selected from the following (1) to (13):
(1) halogen atom,
(2) C₁₋₆ alkyl group which may optionally be substituted with the same or different 1 to 5 substituents selected from Group B,
(3) C₃₋₇ cycloalkyl group which may optionally be substituted with the same or different 1 to 5 substituents selected from Group B,
(4) C₂₋₆ alkenyl group,
(5) cyano group,
(6) —C(═O)—OR^{dd1},
(7) —C(═O)—NR^{dd2}R^{dd3},
(8) —OR^{dd4},
(9) —NR^{dd5}—C(═O)—R^{dd6},
(10) —NR^{dd7}—C(═O)—NR^{dd8}R^{dd9},
(11) —NR^{dd10}—S(═O)₂—R^{dd11},
(12) —NR^{dd12}—S(═O)₂—NR^{dd13}R^{dd14},
(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
R^{e} is hydrogen atom or C₁₋₆ alkyl group;
each R^{j} is the same or different C₁₋₁₂ alkyl group which may optionally be substituted with the same or different 1 to 5 substituents selected from Group A;
each R^{w} is the same or different and selected from the following (1) to (17):
(1) C₁₋₁₂ alkyl group which may optionally be substituted with the same or different 1 to 5 substituents selected from the group consisting of hydroxyl group and halogen atom,
(2) unsaturated heteromonocyclic group which may optionally be substituted with the same or different 1 to 5 C₁₋₆ alkyl groups wherein the unsaturated heteromonocyclic ring has an unsaturated bond, comprises the same or different 1 to 4 hetero atoms selected from nitrogen atom, oxygen atom, or sulfur atom besides carbon atom, and is 5 to 7-membered,
(3) —(CH₂)_{wn1}—C(═O)—OR^{ww1},
(4) —(CH₂)_{wn1}—C(═O)—(CH₂)_{wn1}—NR^{ww2}R^{ww3},
(5) —(CH₂)_{wn1}—C(═O)—(CH₂)_{wn1}—C(═O)—OR^{ww4},
(6) —NR^{ww5}R^{ww6},
(7) —OR^{ww7},
(8) —C(═O)—R^{ww8},
(9) —S(═O)₂—R^{ww9},
(10) ═O,
(11) methylene,
(12) —(CH₂)_{wn2}-ring P,
(13) halogen atom,
(14) —NR^{ww10}—C(═O)—R^{ww11},
(15) —NR^{ww12}—C(═O)—NR^{ww13}R^{ww14},
(16) —NR^{ww15}—S(═O)₂—R^{ww16},
(17) —NR^{ww17}—S(═O)₂—NR^{ww18}R^{ww19};
R^{ww1}, R^{ww2}, R^{ww3}, R^{ww4}, R^{ww5}, R^{ww6} and R^{ww7} are each independently hydrogen atom or C₁₋₆ alkyl group;
R^{ww8} is each independently
hydrogen atom,
C₁₋₆ alkyl group which may optionally be substituted with the same or different 1 to 5 substituents selected from the group consisting of hydroxyl group and halogen atom, or
saturated heteromonocyclic group which may optionally be substituted with the same or different 1 to 5 C₁₋₆ alkyl groups wherein the saturated heteromonocyclic ring comprises the same or different 1 to 4 hetero atoms selected from nitrogen atom, oxygen atom, or sulfur atom besides carbon atom, and is 4 to 6-membered;
R^{ww9} is each independently C₁₋₆ alkyl group which may optionally be substituted with the same or different 1 to 5 halogen atoms;
R^{ww10}, R^{ww11}, R^{ww12}, R^{ww13}, R^{ww14}, R^{ww15}, R^{ww16}, R^{ww17}, R^{ww18}, and R^{ww19} are each independently hydrogen atom or C₁₋₆ alkyl group;
ring P is monocyclic heterocycle which is a carboxylic acid equivalent wherein the carboxylic acid equivalent may optionally be substituted;
wn1 is each independently an integer selected from 0 or 1 to 3; and
wn2 is each independently an integer selected from 1 to 3;
Q is selected from the following (1) to (9):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,
(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;
Y is selected from the following (1) to (3):
(1) single bond,
(2) —S(═O)₂—,
(3) C₁₋₃ alkylene which may optionally be substituted with 1 to 3 hydroxyl groups;
cyclic moiety W is selected from the following (1) to (3):
(1) pyrrolidinyl,
(2) piperidinyl,
(3) C₃₋₇ cycloalkyl;
cn is each independently an integer selected from 0 or 1 to 3;
m is each independently an integer selected from 0 or 1 to 5;
n^{j} is each independently 0, 1 or 2;
Group A consists of the following (a) to (m):
(a) C₁₋₇ alkyl group which may optionally be substituted with the same or different 1 to 5 substituents selected from Group AA,
(b) halogen atom,
(c) phenyl group,
(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,
(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(g) C₃₋₇ cycloalkyl group which may optionally be substituted with the same or different 1 to 5 substituents selected from Group AA,
(h) —C(═O)—NR^{A1}R^{A2},
(i) —C(═O)—OR^{A3},
(j) —C(═O)—R^{A4},
(k) —OR^{A5},
(l) —NR^{A6}R^{A7},
(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
hydrogen atom,
C₁₋₆ alkyl group which may optionally be substituted with the same or different 1 to 6 halogen atoms,
C₃₋₇ cycloalkyl group which may optionally be substituted with the same or different 1 to 5 substituents selected from Group AA,
benzyl group which may optionally be substituted with the same or different 1 to 5 substituents selected from Group AA,
phenyl group which may optionally be substituted with the same or different 1 to 5 substituents selected from Group AA,
C₁₋₆ alkylcarbonyl group,
C₁₋₆ alkylsulfonyl group, or
C₂₋₆ alkenyl group; and
R^{A8} is each independently C₁₋₆ alkyl group;
Group AA consists of the following (a) to (o):
(a) halogen atom,
(b) C₁₋₃ alkyl group which may optionally be substituted with the same or different 1 to 6 halogen atoms,
(c) —OR^{AA1},
(d) —C(═O)—NR^{AA2}R^{AA3},
(e) —C(═O)—OR^{AA4},
(f) —O—C(═O)—R^{AA5},
(g) —C(═O)—R^{AA6},
(h) ═O,
(i) C₃₋₇ cycloalkyl group,
(j) phenyl group which may optionally be substituted with the same or different 1 to 5 C₁₋₃ alkyl groups,
(k) —NR^{AA7}R^{AA8},
(l) —NR^{AA9}—C(═O)—R^{AA10},
(m) —NR^{AA11}—C(═O)—NR^{AA12}R^{AA13},
(n) —NR^{AA14}—S(═O)₂—R^{AA15},
(o) —NR^{AA16}—S(═O)₂—NR^{AA17}R^{AA18};
R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):
(a) halogen atom,
(b) C₃₋₇ cycloalkyl group
(c) —OR^{B1},
(d) —C(═O)—NR^{B2}R^{B3},
(e) —C(═O)—OR^{B4},
(f) C₁₋₆ alkyl group,
(g) —NR^{B5}R^{B6},
(h) —NR^{B7}—C(═O)—R^{B8},
(i) —NR^{B9}—C(═O)—NR^{B10}R^{B11},
(j) —NR^{B12}—S(═O)₂—R^{B13},
(k) —NR^{B14}—S(═O)₂—NR^{B15}R^{B16};
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently, hydrogen atom or C₁₋₆ alkyl group;
provided that when R^{b} is hydrogen atom,
Q is selected from the following (1) to (5):
(1) phenyl group,
(2) C₃₋₇ cycloalkyl group,
(3) C₉₋₁₀ fused carbocyclic group,
(4) cross-linked C₅₋₁₂ cycloalkyl group,
(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;
and further provided that when
is
cyclic moiety W is selected from the following (1) or (2):
(1) pyrrolidinyl,
(2) piperidinyl.

18. A compound represented by the following formulas:
or a pharmaceutically acceptable salt thereof, wherein
is unsaturated heteromonocyclic group selected from the following (i) to (v):
R^{a} is selected from the following (1) to (12):
(1) C₁₋₁₂ alkyl group which may optionally be substituted with the same or different 1 to 5 substituents selected from Group A,
(2) C₃₋₇ cycloalkyl group which may optionally be substituted with the same or different 1 to 5 substituents selected from Group A,
(3) C₂₋₁₂ alkenyl group which may optionally be substituted with the same or different 1 to 5 substituents selected from Group A,
(4) C₂₋₁₂ alkynyl group which may optionally be substituted with the same or different 1 to 5 substituents selected from Group A,
(5) C₅₋₁₁ spirocyclic cycloalkyl group which may optionally be substituted with the same or different 1 to 5 substituents selected from Group A,
(6) cross-linked C₅₋₁₂ cycloalkyl group which may optionally be substituted with the same or different 1 to 5 substituents selected from Group A,
(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,
(8) phenyl group which may optionally be substituted with the same or different 1 to 5 substituents selected from Group A,
(9) C₉₋₁₀ fused carbocyclic group which may optionally be substituted with the same or different 1 to 5 substituents selected from Group A,
(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,
(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;
R^{b} is selected from the following (1) to (6):
(1) C₁₋₆ alkyl group which may optionally be substituted with the same or different 1 to 5 halogen atoms,
(2) C₃₋₇ cycloalkyl group which may optionally be substituted with the same or different 1 to 5 substituents selected from the group consisting of halogen atom and C₁₋₆ alkyl group,
(3) —CH═CH—C(═O)—OR^{bb1},
(4) —CH₂—CH₂—C(═O)—OR^{bb2},
(5) —CH₂—O—CH₂—C(═O)—OR^{bb3},
(6) hydrogen atom;
R^{bb1}, R^{bb2}, and R^{bb3} are each independently hydrogen atom or C₁₋₆ alkyl group;
R^{c} is selected from the following (1) to (17):
(1) —(CH₂)ₙ₁—C(═O)—OR^{cc1},
(2) —O—(CH₂)ₙ₂—C(═O)—OR^{cc2},
(3) —(CH₂)ₙ₃-ring P,
(4) —(CH₂)ₙ₄—C(═O)—NH—S(═O)₂—CH₃,
(5) C₁₋₆ alkyl group which may optionally be substituted with the same or different 1 to 5 substituents selected from Group C,
(6) C₃₋₆ cycloalkyl group which may optionally be substituted with the same or different 1 to 5 substituents selected from Group C,
(7) C₂₋₁₂ alkenyl group,
(8) C₂₋₁₂ alkynyl group,
(9) —NR^{cc3}R^{cc4},
(10) —OR^{cc5},
(11) —O—CH₂CH₂—OH,
(12) —O—CH₂C(═O)NH—CH₃,
R^{cc1} and R^{cc2} are each independently hydrogen atom or C₁₋₆ alkyl group;
R^{cc3} and R^{cc4} are each independently,
hydrogen atom,
C₁₋₆ alkyl group,
C₁₋₆ alkylcarbonyl group,
benzyloxycarbonyl group, or
C₁₋₆ alkyl group which is substituted with 1 to 3 hydroxyl groups;
R^{cc5} is
hydrogen atom,
C₁₋₆ alkyl group which may optionally be substituted with the same or different 1 to 5 substituents selected from Group C, or
benzyl group;
ring P is monocyclic heterocycle which is a carboxylic acid equivalent wherein the carboxylic acid equivalent may optionally be substituted;
n1 is an integer selected from 0 or 1 to 3;
n2 is an integer selected from 0 or 1 to 3;
n3 is an integer selected from 1 to 3; and
n4 is an integer selected from 1 to 3;
each R^{d} is the same or different and selected from the following (1) to (13):
(1) halogen atom,
(2) C₁₋₆ alkyl group which may optionally be substituted with the same or different 1 to 5 substituents selected from Group B,
(3) C₃₋₇ cycloalkyl group which may optionally be substituted with the same or different 1 to 5 substituents selected from Group B,
(4) C₂₋₆ alkenyl group,
(5) cyano group,
(6) —C(═O)—OR^{dd1},
(7) —C(═O)—NR^{dd2}R^{dd3},
(8) —OR^{dd4},
(9) —NR^{dd5}—C(═O)—R^{dd6},
(10) —NR^{dd7}—C(═O)—NR^{dd8}R^{dd9},
(11) —NR^{dd10}—S(═O)₂—R^{dd11},
(12) —NR^{dd12}—S(═O)₂—NR^{dd13}R^{dd14},
(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
R^{c} is hydrogen atom or C₁₋₆ alkyl group;
each R^{j} is the same or different C₁₋₁₂ alkyl group which may optionally be substituted with the same or different 1 to 5 substituents selected from Group A;
Q is selected from the following (1) to (9):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,
(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;
Y is selected from the following (1) to (3):
(1) single bond,
(2) —S(═O)₂—,
(3) C₁₋₃ alkylene which may optionally be substituted with 1 to 3 hydroxyl groups;
m is each independently an integer selected from 0 or 1 to 5;
n^{j} is each independently 0, 1 or 2;
Group A consists of the following (a) to (m):
(a) C₁₋₇ alkyl group which may optionally be substituted with the same or different 1 to 5 substituents selected from Group AA,
(b) halogen atom,
(c) phenyl group,
(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,
(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(g) C₃₋₇ cycloalkyl group which may optionally be substituted with the same or different 1 to 5 substituents selected from Group AA,
(h) —C(═O)—NR^{A1}R^{A2},
(i) —C(═O)—OR^{A3},
(j) —C(═O)—R^{A4},
(k) —OR^{A5},
(l) —NR^{A6}R^{A7},
(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
hydrogen atom,
C₁₋₆ alkyl group which may optionally be substituted with the same or different 1 to 6 halogen atoms,
C₃₋₇ cycloalkyl group which may optionally be substituted with the same or different 1 to 5 substituents selected from Group AA,
benzyl group which may optionally be substituted with the same or different 1 to 5 substituents selected from Group AA,
phenyl group which may optionally be substituted with the same or different 1 to 5 substituents selected from Group AA,
C₁₋₆ alkylcarbonyl group,
C₁₋₆ alkylsulfonyl group, or
C₂₋₆ alkenyl group; and
R^{A8} is each independently C₁₋₆ alkyl group;
Group AA consists of the following (a) to (o):
(a) halogen atom,
(b) C₁₋₃ alkyl group which may optionally be substituted with the same or different 1 to 6 halogen atoms,
(c) —OR^{AA1},
(d) —C(═O)—NR^{AA2}R^{AA3},
(e) —C(═O)—OR^{AA4},
(f) —O—C(═O)—R^{AA5},
(g) —C(═O)—R^{AA6},
(h) ═O,
(i) C₃₋₇ cycloalkyl group,
(j) phenyl group which may optionally be substituted with the same or different 1 to 5 C₁₋₃ alkyl groups,
(k) —NR^{AA7}R^{AA8},
(l) —NR^{AA9}—C(═O)—R^{AA10},
(m) —NR^{AA11}—C(═O)—NR^{AA12}R^{AA13},
(n) —NR^{AA14}—S(═O)₂—R^{AA15},
(o) —NR^{AA16}—S(═O)₂—NR^{AA17}R^{AA18};
R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):
(a) halogen atom,
(b) C₃₋₇ cycloalkyl group
(c) —OR^{B1},
(d) —C(═O)—NR^{B2}R^{B3},
(e) —C(═O)—OR^{B4},
(f) C₁₋₆ alkyl group,
(g) —NR^{B5}R^{B6},
(h) —NR^{B7}—C(═O)—R^{B8},
(i) —NR^{B9}—C(═O)—NR^{B10}R^{B11},
(j) —NR^{B12}—S(═O)₂—R^{B13},
(k) —NR^{B14}—S(═O)₂—NR^{B15}R^{B16};
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently hydrogen atom or C₁₋₆ alkyl group;
Group C consists of the following (a) to (k):
(a) phenyl group which may optionally be substituted with the same or different 1 to 5 substituents selected from Group CC,
(b) cyano group,
(c) halogen atom,
(d) —OR^{CD1},
(e) —NR^{CD2}R^{CD3},
(f) —C(═O)—NR^{CD4}R^{CD5},
(i) saturated heteromonocyclic group which may optionally be substituted with the same or different 1 to 5 substituents selected from Group CC wherein the saturated heteromonocyclic ring comprises the same or different 1 to 4 hetero atoms selected from nitrogen atom, oxygen atom, or sulfur atom besides carbon atom, and is 4 to 6-membered, excluding ring P,
(j) unsaturated heteromonocyclic group which may optionally be substituted with the same or different 1 to 5 substituents selected from Group CC wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered, excluding ring P,
R^{CD1}, R^{CD2}, and R^{CD3} are each independently,
hydrogen atom,
C₁₋₆ alkyl group,
C₁₋₆ alkylcarbonyl group,
benzyl group,
—C(═O)—O—CH₂-phenyl,
—C(═O)—N(CH₃)₂,
—C(═O)—C(OH)(CH₃)₂,
—C(═O)—CH₂—O—CH₃, or
—C(═O)—CH₂—CN; and
R^{CD4} and R^{CD5} are each independently hydrogen atom or C₁₋₆ alkyl group;
Group CC consists of the following (a) to (c):
(a) halogen atom,
(b) C₁₋₃ alkyl group which may optionally be substituted with the same or different 1 to 6 halogen atoms,
(c) —OR^{AA1},
R^{AA1} is each independently hydrogen atom or C₁₋₆ alkyl group;
provided that when R^{a} is
(1) phenyl group which may optionally be substituted with the same or different 1 to 5 substituents selected from Group A,
(2) C₉₋₁₀ fused carbocyclic group which may optionally be substituted with the same or different 1 to 5 substituents selected from Group A, or
(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
Q is selected from the following (1) to (6):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;
and further provided that when R^{b} is hydrogen atom,
Q is selected from the following (1) to (5):
(1) phenyl group,
(2) C₃₋₇ cycloalkyl group,
(3) C₉₋₁₀ fused carbocyclic group,
(4) cross-linked C₅₋₁₂ cycloalkyl group,
(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered.

19. A compound represented by the following formulas:
or a pharmaceutically acceptable salt thereof, wherein
is unsaturated heteromonocyclic group selected from the following (i) to (v):
R^{a} is selected from the following (1) to (12):
(1) C₁₋₁₂ alkyl group which may optionally be substituted with the same or different 1 to 5 substituents selected from Group A,
(2) C₃₋₇ cycloalkyl group which may optionally be substituted with the same or different 1 to 5 substituents selected from Group A,
(3) C₂₋₁₂ alkenyl group which may optionally be substituted with the same or different 1 to 5 substituents selected from Group A,
(4) C₂₋₁₂ alkynyl group which may optionally be substituted with the same or different 1 to 5 substituents selected from Group A,
(5) C₅₋₁₁ spirocyclic cycloalkyl group which may optionally be substituted with the same or different 1 to 5 substituents selected from Group A,
(6) cross-linked C₅₋₁₂ cycloalkyl group which may optionally be substituted with the same or different 1 to 5 substituents selected from Group A,
(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,
(8) phenyl group which may optionally be substituted with the same or different 1 to 5 substituents selected from Group A,
(9) C₉₋₁₀ fused carbocyclic group which may optionally be substituted with the same or different 1 to 5 substituents selected from Group A,
(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,
(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;
R^{b} is selected from the following (1) to (6):
(1) C₁₋₆ alkyl group which may optionally be substituted with the same or different 1 to 5 halogen atoms,
(2) C₃₋₇ cycloalkyl group which may optionally be substituted with the same or different 1 to 5 substituents selected from the group consisting of halogen atom and C₁₋₆ alkyl group,
(3) —CH═CH—C(═O)—OR^{bb1},
(4) —CH₂—CH₂—C(═O)—OR^{bb2},
(5) —CH₂—O—CH₂—C(═O)—OR^{bb3},
(6) hydrogen atom;
R^{bb1}, R^{bb2}, and R^{bb3} are each independently hydrogen atom or C₁₋₆ alkyl group;
R^{c} is selected from the following (1) to (17):
(1) —(CH₂)ₙ₁—C(═O)—OR^{cc1},
(2) —O—(CH₂)—C(═O)—OR^{cc2},
(3) —(CH₂)ₙ₃-ring P,
(4) —(CH₂)ₙ₄—C(═O)—NH—S(═O)₂—CH₃,
(5) C₁₋₆ alkyl group which may optionally be substituted with the same or different 1 to 5 substituents selected from Group C,
(6) C₃₋₆ cycloalkyl group which may optionally be substituted with the same or different 1 to 5 substituents selected from Group C,
(7) C₂₋₁₂ alkenyl group,
(8) C₂₋₁₂ alkynyl group,
(9) —NR^{cc3}R^{cc4},
(10) —OR^{cc5},
(11) —O—CH₂CH₂—OH,
(12) —O—CH₂C(═O)NH—CH₃,
R^{cc1} and R^{cc2} are each independently hydrogen atom or C₁₋₆ alkyl group;
R^{cc3} and R^{cc4} are each independently,
hydrogen atom,
C₁₋₆ alkyl group,
C₁₋₆ alkylcarbonyl group,
benzyloxycarbonyl group, or
C₁₋₆ alkyl group which is substituted with 1 to 3 hydroxyl groups;
R^{cc5} is
hydrogen atom,
C₁₋₆ alkyl group which may optionally be substituted with the same or different 1 to 5 substituents selected from Group C, or
benzyl group;
ring P is monocyclic heterocycle which is a carboxylic acid equivalent wherein the carboxylic acid equivalent may optionally be substituted;
n1 is an integer selected from 0 or 1 to 3;
n2 is an integer selected from 0 or 1 to 3;
n3 is an integer selected from 1 to 3; and
n4 is an integer selected from 1 to 3;
each R^{d} is the same or different and selected from the following (1) to (13):
(1) halogen atom,
(2) C₁₋₆ alkyl group which may optionally be substituted with the same or different 1 to 5 substituents selected from Group B,
(3) C₃₋₇ cycloalkyl group which may optionally be substituted with the same or different 1 to 5 substituents selected from Group B,
(4) C₂₋₆ alkenyl group,
(5) cyano group,
(6) —C(═O)—OR^{dd1},
(7) —C(═O)—NR^{dd2}R^{dd3},
(8) —OR^{dd4},
(9) —NR^{dd5}—C(═O)—R^{dd6},
(10) —NR^{dd7}—C(═O)—NR^{dd8}R^{dd9},
(11) —NR^{dd10}—S(═O)₂—R^{dd11},
(12) —NR^{dd12}—S(═O)₂—NR^{dd13}R^{dd14},
(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
Re is hydrogen atom or C₁₋₆ alkyl group;
Rf is hydrogen atom or C₁₋₆ alkyl group;
each Rj is the same or different C₁₋₁₂ alkyl group which may optionally be substituted with the same or different 1 to 5 substituents selected from Group A;
Q is selected from the following (1) to (9):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,
(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;
Y is selected from the following (1) to (3):
(1) single bond,
(2) —S(═O)₂—,
(3) C₁₋₃ alkylene which may optionally be substituted with 1 to 3 hydroxyl groups;
m is each independently an integer selected from 0 or 1 to 5;
n^{j} is each independently 0, 1 or 2;
Group A consists of the following (a) to (m):
(a) C₁₋₇ alkyl group which may optionally be substituted with the same or different 1 to 5 substituents selected from Group AA,
(b) halogen atom,
(c) phenyl group,
(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,
(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(g) C₃₋₇ cycloalkyl group which may optionally be substituted with the same or different 1 to 5 substituents selected from Group AA,
(h) —C(═O)—NR^{A1}R^{A2},
(i) —C(═O)—OR^{A3},
(j) —C(═O)—R^{A4},
(k) —OR^{A5},
(l) —NR^{A6}R^{A7},
(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
hydrogen atom,
C₁₋₆ alkyl group which may optionally be substituted with the same or different 1 to 6 halogen atoms,
C₃₋₇ cycloalkyl group which may optionally be substituted with the same or different 1 to 5 substituents selected from Group AA,
benzyl group which may optionally be substituted with the same or different 1 to 5 substituents selected from Group AA,
phenyl group which may optionally be substituted with the same or different 1 to 5 substituents selected from Group AA,
C₁₋₆ alkylcarbonyl group,
C₁₋₆ alkylsulfonyl group, or
C₂₋₆ alkenyl group; and
R^{A8} is each independently C₁₋₆ alkyl group;
Group AA consists of the following (a) to (o):
(a) halogen atom,
(b) C₁₋₃ alkyl group which may optionally be substituted with the same or different 1 to 6 halogen atoms,
(c) —OR^{AA1},
(d) —C(═O)—NR^{AA2}R^{AA3},
(e) —C(═O)—OR^{AA4},
(f) —O—C(═O)—R^{AA5},
(g) —C(═O)—R^{AA6},
(h) ═O,
(i) C₃₋₇ cycloalkyl group,
(j) phenyl group which may optionally be substituted with the same or different 1 to 5 C₁₋₃ alkyl groups,
(k) —NR^{AA7}R^{AA8},
(l) —NR^{AA9}—C(═O)—R^{AA10},
(m) —NR^{AA11}—C(═O)—NR^{AA12}R^{AA13},
(n) —NR^{AA14}—S(═O)₂—R^{AA15},
(o) —NR^{AA16}—S(═O)₂—NR^{AA17}R^{AA18};
R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):
(a) halogen atom,
(b) C₃₋₇ cycloalkyl group
(c) —OR^{B1},
(d) —C(═O)—NR^{B2}R^{B3},
(e) —C(═O)—OR^{B4},
(f) C₁₋₆ alkyl group,
(g) —NR^{B5}R^{B6},
(h) —NR^{B7}—C(═O)—R^{B8},
(i) —NR^{B9}—C(═O)—NR^{B10}R^{B11},
(j) —NR^{B12}—S(═O)₂—R^{B13},
(k) —NR^{B14}—S(═O)₂—NR^{B15}R^{B16},
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently hydrogen atom or C₁₋₆ alkyl group;
Group C consists of the following (a) to (k):
(a) phenyl group which may optionally be substituted with the same or different 1 to 5 substituents selected from Group CC,
(b) cyano group,
(c) halogen atom,
(d) —OR^{CD1},
(e) —NR^{CD2}R^{CD3},
(f) —C(═O)—NR^{CD4}R^{CD5},
(i) saturated heteromonocyclic group which may optionally be substituted with the same or different 1 to 5 substituents selected from Group CC wherein the saturated heteromonocyclic ring comprises the same or different 1 to 4 hetero atoms selected from nitrogen atom, oxygen atom, or sulfur atom besides carbon atom, and is 4 to 6-membered, excluding ring P,
(j) unsaturated heteromonocyclic group which may optionally be substituted with the same or different 1 to 5 substituents selected from Group CC wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered, excluding ring P,
R^{CD1}, R^{CD2}, and R^{CD3} are each independently,
hydrogen atom,
C₁₋₆ alkyl group,
C₁₋₆ alkylcarbonyl group,
benzyl group,
—C(═O)—O—CH₂-phenyl,
—C(═O)—N(CH₃)₂,
—C(═O)—C(OH)(CH₃)₂,
—C(═O)—CH₂—O—CH₃, or
—C(═O)—CH₂—CN; and
R^{CD4} and R^{CD5} are each independently hydrogen atom or C₁₋₆ alkyl group;
Group CC consists of the following (a) to (c):
(a) halogen atom,
(b) C₁₋₃ alkyl group which may optionally be substituted with the same or different 1 to 6 halogen atoms,
(c) —OR^{AA1},
R^{AA1} is each independently hydrogen atom or C₁₋₆ alkyl group;
provided that when R^{a} is
(1) phenyl group which may optionally be substituted with the same or different 1 to 5 substituents selected from Group A,
(2) C₉₋₁₀ fused carbocyclic group which may optionally be substituted with the same or different 1 to 5 substituents selected from Group A, or
(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
Q is selected from the following (1) to (6):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;
further provided that when R^{b} is hydrogen atom,
Q is selected from the following (1) to (5):
(1) phenyl group,
(2) C₃₋₇ cycloalkyl group,
(3) C₉₋₁₀ fused carbocyclic group,
(4) cross-linked C₅₋₁₂ cycloalkyl group,
(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;
and further provided that when
is
Q is selected from the following (1) to (6):
(1) C₃₋₇ cycloalkyl group,
(2) C₉₋₁₀ fused carbocyclic group selected from the group consisting of indanyl group and 1,2,3,4-tetrahydronaphthyl group,
(3) cross-linked C₅₋₁₂ cycloalkyl group,
(4) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,
(5) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(6) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered.

20. A compound represented by the following formulas:
or a pharmaceutically acceptable salt thereof, wherein
is unsaturated heteromonocyclic group selected from the following (i) to (v):
R^{a} is selected from the following (1) to (12):
(1) C₁₋₁₂ alkyl group which may optionally be substituted with the same or different 1 to 5 substituents selected from Group A,
(2) C₃₋₇ cycloalkyl group which may optionally be substituted with the same or different 1 to 5 substituents selected from Group A,
(3) C₂₋₁₂ alkenyl group which may optionally be substituted with the same or different 1 to 5 substituents selected from Group A,
(4) C₂₋₁₂ alkynyl group which may optionally be substituted with the same or different 1 to 5 substituents selected from Group A,
(5) C₅₋₁₁ spirocyclic cycloalkyl group which may optionally be substituted with the same or different 1 to 5 substituents selected from Group A,
(6) cross-linked C₅₋₁₂ cycloalkyl group which may optionally be substituted with the same or different 1 to 5 substituents selected from Group A,
(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,
(8) phenyl group which may optionally be substituted with the same or different 1 to 5 substituents selected from Group A,
(9) C₉₋₁₀ fused carbocyclic group which may optionally be substituted with the same or different 1 to 5 substituents selected from Group A,
(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,
(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;
R^{b} is selected from the following (1) to (6):
(1) C₁₋₆ alkyl group which may optionally be substituted with the same or different 1 to 5 halogen atoms,
(2) C₃₋₇ cycloalkyl group which may optionally be substituted with the same or different 1 to 5 substituents selected from the group consisting of halogen atom and C₁₋₆ alkyl group,
(3) —CH═CH—C(═O)—OR^{bb1},
(4) —CH₂—CH₂—C(═O)—OR^{bb2},
(5) —CH₂—O—CH₂—C(═O)—OR^{bb3},
(6) hydrogen atom;
R^{bb1}, R^{bb2}, and R^{bb3} are each independently hydrogen atom or C₁₋₆ alkyl group;
R^{c} is selected from the following (1) to (17):
(1) —(CH₂)ₙ₁—C(═O)—OR^{cc1},
(2) —O—(CH₂)—C(═O)—OR^{cc2},
(3) —(CH₂)ₙ₃-ring P,
(4) —(CH₂)ₙ₄—C(═O)—NH—S(═O)₂—CH₃,
(5) C₁₋₆ alkyl group which may optionally be substituted with the same or different 1 to 5 substituents selected from Group C,
(6) C₃₋₆ cycloalkyl group which may optionally be substituted with the same or different 1 to 5 substituents selected from Group C,
(7) C₂₋₁₂ alkenyl group,
(8) C₂₋₁₂ alkynyl group,
(9) —NR^{cc3}R^{cc4},
(10) —OR^{cc5},
(11) —O—CH₂CH₂—OH,
(12) —O—CH₂C(═O)NH—CH₃,
R^{cc1} and R^{cc2} are each independently hydrogen atom or C₁₋₆ alkyl group;
R^{cc3} and R^{cc4} are each independently,
hydrogen atom,
C₁₋₆ alkyl group,
C₁₋₆ alkylcarbonyl group,
benzyloxycarbonyl group, or
C₁₋₆ alkyl group which is substituted with 1 to 3 hydroxyl groups;
R^{cc5} is
hydrogen atom,
C₁₋₆ alkyl group which may optionally be substituted with the same or different 1 to 5 substituents selected from Group C, or
benzyl group;
ring P is monocyclic heterocycle which is a carboxylic acid equivalent wherein the carboxylic acid equivalent may optionally be substituted;
n1 is an integer selected from 0 or 1 to 3;
n2 is an integer selected from 0 or 1 to 3;
n3 is an integer selected from 1 to 3; and
n4 is an integer selected from 1 to 3;
each R^{d} is the same or different and selected from the following (1) to (13):
(1) halogen atom,
(2) C₁₋₆ alkyl group which may optionally be substituted with the same or different 1 to 5 substituents selected from Group B,
(3) C₃₋₇ cycloalkyl group which may optionally be substituted with the same or different 1 to 5 substituents selected from Group B,
(4) C₂₋₆ alkenyl group,
(5) cyano group,
(6) —C(═O)—OR^{dd1},
(7) —C(═O)—NR^{dd2}R^{dd3},
(8) —OR^{dd4},
(9) —NR^{dd5}—C(═O)—R^{dd6},
(10) —NR^{dd7}—C(═O)—NR^{dd8}R^{dd9},
(11) —NR^{dd10}—S(═O)₂—R^{dd11},
(12) —NR^{dd12}—S(═O)₂—NR^{dd13}R^{dd14},
(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
R^{e} is hydrogen atom or C₁₋₆ alkyl group;
R^{f} is hydrogen atom or C₁₋₆ alkyl group;
each R^{j} is the same or different C₁₋₁₂ alkyl group which may optionally be substituted with the same or different 1 to 5 substituents selected from Group A;
Q is selected from the following (1) to (9):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,
(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;
Y is selected from the following (1) to (3):
(1) single bond,
(2) —S(═O)₂—,
(3) C₁₋₃ alkylene which may optionally be substituted with 1 to 3 hydroxyl groups;
m is each independently an integer selected from 0 or 1 to 5;
n^{j} is each independently 0, 1 or 2;
Group A consists of the following (a) to (m):
(a) C₁₋₇ alkyl group which may optionally be substituted with the same or different 1 to 5 substituents selected from Group AA,
(b) halogen atom,
(c) phenyl group,
(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,
(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(g) C₃₋₇ cycloalkyl group which may optionally be substituted with the same or different 1 to 5 substituents selected from Group AA,
(h) —C(═O)—NR^{A1}R^{A2},
(i) —C(═O)—OR^{A3},
(j) —C(═O)—R^{A4},
(k) —OR^{A5},
(l) —NR^{A6}R^{A7},
(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
hydrogen atom,
C₁₋₆ alkyl group which may optionally be substituted with the same or different 1 to 6 halogen atoms,
C₃₋₇ cycloalkyl group which may optionally be substituted with the same or different 1 to 5 substituents selected from Group AA,
benzyl group which may optionally be substituted with the same or different 1 to 5 substituents selected from Group AA,
phenyl group which may optionally be substituted with the same or different 1 to 5 substituents selected from Group AA,
C₁₋₆ alkylcarbonyl group,
C₁₋₆ alkylsulfonyl group, or
C₂₋₆ alkenyl group; and
R^{A8} is each independently C₁₋₆ alkyl group;
Group AA consists of the following (a) to (o):
(a) halogen atom,
(b) C₁₋₃ alkyl group which may optionally be substituted with the same or different 1 to 6 halogen atoms,
(c) —OR^{AA1},
(d) —C(═O)—NR^{AA2}R^{AA3},
(e) —C(═O)—OR^{AA4},
(f) —O—C(═O)—R^{AA5},
(g) —C(═O)—R^{AA6},
(h) ═O,
(i) C₃₋₇ cycloalkyl group,
(j) phenyl group which may optionally be substituted with the same or different 1 to 5 C₁₋₃ akyl groups,
(k) —NR^{AA7}R^{AA8},
(l) —NR^{AA9}—C(═O)—R^{AA10},
(m) —NR^{AA11}—C(═O)—NR^{AA12}R^{AA13},
(n) —NR^{AA14}—S(═O)₂—R^{AA15},
(o) —NR^{AA16}—S(═O)₂—NR^{AA17}R^{AA18};
R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):
(a) halogen atom,
(b) C₃₋₇ cycloalkyl group
(c) —OR^{B1},
(d) —C(═O)—NR^{B2}R^{B3},
(e) —C(═O)—OR^{B4},
(f) C₁₋₆ alkyl group,
(g) —NR^{B5}R^{B6},
(h) —NR^{B7}—C(═O)—R^{B8},
(i) —NR^{B9}—C(═O)—NR^{B10}R^{B11},
(j) —NR^{B12}—S(═O)₂—R^{B13},
(k) —NR^{B14}—S(═O)₂—NR^{B15}R^{B16},
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently hydrogen atom or C₁₋₆ alkyl group;
Group C consists of the following (a) to (k):
(a) phenyl group which may optionally be substituted with the same or different 1 to 5 substituents selected from Group CC,
(b) cyano group,
(c) halogen atom,
(d) —OR^{CD1},
(e) —NR^{CD2}R^{CD3},
(f) —C(═O)—NR^{CD4}R^{CD5},
(i) saturated heteromonocyclic group which may optionally be substituted with the same or different 1 to 5 substituents selected from Group CC wherein the saturated heteromonocyclic ring comprises the same or different 1 to 4 hetero atoms selected from nitrogen atom, oxygen atom, or sulfur atom besides carbon atom, and is 4 to 6-membered, excluding ring P,
(j) unsaturated heteromonocyclic group which may optionally be substituted with the same or different 1 to 5 substituents selected from Group CC wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered, excluding ring P,
R^{CD1}, R^{CD2}, and R^{CD3} are each independently,
hydrogen atom,
C₁₋₆ alkyl group,
C₁₋₆ alkylcarbonyl group,
benzyl group,
—C(═O)—O—CH₂-phenyl,
—C(═O)—N(CH₃)₂,
—C(═O)—C(OH)(CH₃)₂,
—C(═O)—CH₂—O—CH₃, or
—C(═O)—CH₂—CN; and
R^{CD4} and R^{CD5} are each independently hydrogen atom or C₁₋₆ alkyl group;
Group CC consists of the following (a) to (c):
(a) halogen atom,
(b) C₁₋₃ alkyl group which may optionally be substituted with the same or different 1 to 6 halogen atoms,
(c) —OR^{AA1},
R^{AA1} is each independently hydrogen atom or C₁₋₆ alkyl group;
provided that when R^{a} is
(1) phenyl group which may optionally be substituted with the same or different 1 to 5 substituents selected from Group A,
(2) C₉₋₁₀ fused carbocyclic group which may optionally be substituted with the same or different 1 to 5 substituents selected from Group A, or
(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
Q is selected from the following (1) to (6):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;
further provided that when R^{b} is hydrogen atom,
Q is selected from the following (1) to (5):
(1) phenyl group,
(2) C₃₋₇ cycloalkyl group,
(3) C₉₋₁₀ fused carbocyclic group,
(4) cross-linked C₅₋₁₂ cycloalkyl group,
(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;
and further provided that when
is
R^{a} is selected from the following (1) or (2):
(1) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,
(2) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered.

21. A compound represented by the following formulas:
or a pharmaceutically acceptable salt thereof, wherein
is unsaturated heteromonocyclic group selected from the following (i) to (v):
R^{a} is selected from the following (1) to (12):
(1) C₁₋₁₂ alkyl group which may optionally be substituted with the same or different 1 to 5 substituents selected from Group A,
(2) C₃₋₇ cycloalkyl group which may optionally be substituted with the same or different 1 to 5 substituents selected from Group A,
(3) C₂₋₁₂ alkenyl group which may optionally be substituted with the same or different 1 to 5 substituents selected from Group A,
(4) C₂₋₁₂ alkynyl group which may optionally be substituted with the same or different 1 to 5 substituents selected from Group A,
(5) C₅₋₁₁ spirocyclic cycloalkyl group which may optionally be substituted with the same or different 1 to 5 substituents selected from Group A,
(6) cross-linked C₅₋₁₂ cycloalkyl group which may optionally be substituted with the same or different 1 to 5 substituents selected from Group A,
(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,
(8) phenyl group which may optionally be substituted with the same or different 1 to 5 substituents selected from Group A,
(9) C₉₋₁₀ fused carbocyclic group which may optionally be substituted with the same or different 1 to 5 substituents selected from Group A,
(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,
(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;
R^{b} is selected from the following (1) to (6):
(1) C₁₋₆ alkyl group which may optionally be substituted with the same or different 1 to 5 halogen atoms,
(2) C₃₋₇ cycloalkyl group which may optionally be substituted with the same or different 1 to 5 substituents selected from the group consisting of halogen atom and C₁₋₆ alkyl group,
(3) —CH═CH—C(═O)—OR^{bb1},
(4) —CH₂—CH₂—C(═O)—OR^{bb2},
(5) —CH₂—O—CH₂—C(═O)—OR^{bb3},
(6) hydrogen atom;
R^{bb1}, R^{bb2}, and R^{bb3} are each independently hydrogen atom or C₁₋₆ alkyl group;
R^{c} is selected from the following (1) to (17):
(1) —(CH₂)ₙ₁—C(═O)—OR^{cc1},
(2) —O—(CH₂)ₙ₂—C(═O)—OR^{cc2},
(3) —(CH₂)ₙ₃-ring P,
(4) —(CH₂)ₙ₄—C(═O)—NH—S(═O)₂—CH₃,
(5) C₁₋₆ alkyl group which may optionally be substituted with the same or different 1 to 5 substituents selected from Group C,
(6) C₃₋₆ cycloalkyl group which may optionally be substituted with the same or different 1 to 5 substituents selected from Group C,
(7) C₂₋₁₂ alkenyl group,
(8) C₂₋₁₂ alkynyl group,
(9) —NR^{cc3}R^{cc4},
(10) —OR^{cc5},
(11) —O—CH₂CH₂—OH,
(12) —O—CH₂C(═O)NH—CH₃,
R^{cc1} and R^{cc2} are each independently hydrogen atom or C₁₋₆ alkyl group;
R^{cc3} and R^{cc4} are each independently,
hydrogen atom,
C₁₋₆ alkyl group,
C₁₋₆ alkylcarbonyl group,
benzyloxycarbonyl group, or
C₁₋₆ alkyl group which is substituted with 1 to 3 hydroxyl groups;
R^{cc5} is
hydrogen atom,
C₁₋₆ alkyl group which may optionally be substituted with the same or different 1 to 5 substituents selected from Group C, or
benzyl group;
ring P is monocyclic heterocycle which is a carboxylic acid equivalent wherein the carboxylic acid equivalent may optionally be substituted;
n1 is an integer selected from 0 or 1 to 3;
n2 is an integer selected from 0 or 1 to 3;
n3 is an integer selected from 1 to 3; and
n4 is an integer selected from 1 to 3;
each R^{d} is the same or different and selected from the following (1) to (13):
(1) halogen atom,
(2) C₁₋₆ alkyl group which may optionally be substituted with the same or different 1 to 5 substituents selected from Group B,
(3) C₃₋₇ cycloalkyl group which may optionally be substituted with the same or different 1 to 5 substituents selected from Group B,
(4) C₂₋₆ alkenyl group,
(5) cyano group,
(6) —C(═O)—OR^{dd1},
(7) —C(═O)—NR^{dd2}R^{dd3},
(8) —OR^{dd4},
(9) —NR^{dd5}—C(═O)—R^{dd6},
(10) —NR^{dd7}—C(═O)—NR^{dd8}R^{dd9},
(11) —NR^{dd10}—S(═O)₂—R^{dd11},
(12) —NR^{dd12}—S(═O)₂—NR^{dd13}R^{dd14},
(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
R^{e} is hydrogen atom or C₁₋₆ alkyl group;
R^{f} is hydrogen atom or C₁₋₆ alkyl group;
each R^{j} is the same or different C₁₋₁₂ alkyl group which may optionally be substituted with the same or different 1 to 5 substituents selected from Group A;
Q is selected from the following (1) to (9):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,
(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;
Y is selected from the following (1) to (3):
(1) single bond,
(2) —S(═O)₂—,
(3) C₁₋₃ alkylene which may optionally be substituted with 1 to 3 hydroxyl groups;
m is each independently an integer selected from 0 or 1 to 5;
nj is each independently 0, 1 or 2;
Group A consists of the following (a) to (m):
(a) C₁₋₇ alkyl group which may optionally be substituted with the same or different 1 to 5 substituents selected from Group AA,
(b) halogen atom,
(c) phenyl group,
(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,
(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(g) C₃₋₇ cycloalkyl group which may optionally be substituted with the same or different 1 to 5 substituents selected from Group AA,
(h) —C(═O)—NR^{A1}R^{A2},
(i) —C(═O)—OR^{A3},
(j) —C(═O)—R^{A4},
(k) —OR^{A5},
(l) —NR^{A6}R^{A7},
(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
hydrogen atom,
C₁₋₆ alkyl group which may optionally be substituted with the same or different 1 to 6 halogen atoms,
C₃₋₇ cycloalkyl group which may optionally be substituted with the same or different 1 to 5 substituents selected from Group AA,
benzyl group which may optionally be substituted with the same or different 1 to 5 substituents selected from Group AA,
phenyl group which may optionally be substituted with the same or different 1 to 5 substituents selected from Group AA,
C₁₋₆ alkylcarbonyl group,
C₁₋₆ alkylsulfonyl group, or
C₂₋₆ alkenyl group; and
R^{A8} is each independently C₁₋₆ alkyl group;
Group AA consists of the following (a) to (o):
(a) halogen atom,
(b) C₁₋₃ alkyl group which may optionally be substituted with the same or different 1 to 6 halogen atoms,
(c) —OR^{AA1},
(d) —C(═O)—NR^{AA2}R^{AA3},
(e) —C(═O)—OR^{AA4},
(f) —O—C(═O)—R^{AA5},
(g) —C(═O)—R^{AA6},
(h) ═O,
(i) C₃₋₇ cycloalkyl group,
(j) phenyl group which may optionally be substituted with the same or different 1 to 5 C₁₋₃ alkyl groups,
(k) —NR^{AA7}R^{AA8},
(l) —NR^{AA9}—C(═O)—R^{AA10},
(m) —NR^{AA11}—C(═O)—NR^{AA12}R^{AA13},
(n) —NR^{AA14}—S(═O)₂—R^{AA15},
(o) —NR^{AA16}—S(═O)₂—NR^{AA17}R^{AA18};
R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):
(a) halogen atom,
(b) C₃₋₇ cycloalkyl group
(c) —OR^{B1},
(d) —C(═O)—NR^{B2}R^{B3},
(e) —C(═O)—OR^{B4},
(f) C₁₋₆ alkyl group,
(g) —NR^{B5}R^{B6},
(h) —NR^{B7}—C(═O)—R^{B8},
(i) —NR^{B9}—C(═O)—NR^{B10}R^{B11},
(j) —NR^{B12}—S(═O)₂—R^{B13},
(k) —NR^{B14}—S(═O)₂—NR^{B15}R^{B16},
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently hydrogen atom or C₁₋₆ alkyl group;
Group C consists of the following (a) to (k):
(a) phenyl group which may optionally be substituted with the same or different 1 to 5 substituents selected from Group CC,
(b) cyano group,
(c) halogen atom,
(d) —OR^{CD1},
(e) —NR^{CD2}R^{CD3},
(f) —C(═O)—NR^{CD4}R^{CD5},
(i) saturated heteromonocyclic group which may optionally be substituted with the same or different 1 to 5 substituents selected from Group CC wherein the saturated heteromonocyclic ring comprises the same or different 1 to 4 hetero atoms selected from nitrogen atom, oxygen atom, or sulfur atom besides carbon atom, and is 4 to 6-membered, excluding ring P,
(j) unsaturated heteromonocyclic group which may optionally be substituted with the same or different 1 to 5 substituents selected from Group CC wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered, excluding ring P,
R^{CD1}, R^{CD2}, and R^{CD3} are each independently,
hydrogen atom,
C₁₋₆ alkyl group,
C₁₋₆ alkylcarbonyl group,
benzyl group,
—C(═O)—O—CH₂-phenyl,
—C(═O)—N(CH₃)₂,
—C(═O)—C(OH)(CH₃)₂,
—C(═O)—CH₂—O—CH₃, or
—C(═O)—CH₂—CN; and
R^{CD4} and R^{CD5} are each independently hydrogen atom or C₁₋₆ alkyl group;
Group CC consists of the following (a) to (c):
(a) halogen atom,
(b) C₁₋₃ alkyl group which may optionally be substituted with the same or different 1 to 6 halogen atoms,
(c) —OR^{AA1},
R^{AA1} is each independently hydrogen atom or C₁₋₆ alkyl group;
provided that when R^{a} is
(1) phenyl group which may optionally be substituted with the same or different 1 to 5 substituents selected from Group A,
(2) C₉₋₁₀ fused carbocyclic group which may optionally be substituted with the same or different 1 to 5 substituents selected from Group A, or
(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
Q is selected from the following (1) to (6):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;
further provided that when R^{b} is hydrogen atom,
Q is selected from the following (1) to (5):
(1) phenyl group,
(2) C₃₋₇ cycloalkyl group,
(3) C₉₋₁₀ fused carbocyclic group,
(4) cross-linked C₅₋₁₂ cycloalkyl group,
(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;
and further provided that when
is
R^{c} is selected from the following (1) to (15):
(1) —(CH₂)ₙ₃-ring P,
(2) —(CH₂)ₙ₄—C(═O)—NH—S(═O)₂—CH₃,
(3) C₁₋₆ alkyl group which may optionally be substituted with the same or different 1 to 5 substituents selected from Group C,
(4) C₃₋₆ cycloalkyl group which may optionally be substituted with the same or different 1 to 5 substituents selected from Group C,
(5) C₂₋₁₂ alkenyl group,
(6) C₂₋₁₂ alkynyl group,
(7) —NR^{cc3}R^{cc4},
(8) —OR^{cc5},
(9) —O—CH₂CH₂—OH,
(10) —O—CH₂C(═O)NH—CH₃,

22. A compound represented by the following formula:
or a pharmaceutically acceptable salt thereof, wherein
is unsaturated heteromonocyclic group selected from the following (i) to (ii):
R^{a} is selected from the following (1) to (12):
(1) C₁₋₁₂ alkyl group which may optionally be substituted with the same or different 1 to 5 substituents selected from Group A,
(2) C₃₋₇ cycloalkyl group which may optionally be substituted with the same or different 1 to 5 substituents selected from Group A,
(3) C₂₋₁₂ alkenyl group which may optionally be substituted with the same or different 1 to 5 substituents selected from Group A,
(4) C₂₋₁₂ alkynyl group which may optionally be substituted with the same or different 1 to 5 substituents selected from Group A,
(5) C₅₋₁₁ spirocyclic cycloalkyl group which may optionally be substituted with the same or different 1 to 5 substituents selected from Group A,
(6) cross-linked C₅₋₁₂ cycloalkyl group which may optionally be substituted with the same or different 1 to 5 substituents selected from Group A,
(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,
(8) phenyl group which may optionally be substituted with the same or different 1 to 5 substituents selected from Group A,
(9) C₉₋₁₀ fused carbocyclic group which may optionally be substituted with the same or different 1 to 5 substituents selected from Group A,
(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,
(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;
R^{b} is selected from the following (1) to (6):
(1) C₁₋₆ alkyl group which may optionally be substituted with the same or different 1 to 5 halogen atoms,
(2) C₃₋₇ cycloalkyl group which may optionally be substituted with the same or different 1 to 5 substituents selected from the group consisting of halogen atom and C₁₋₆ alkyl group,
(3) —CH═CH—C(═O)—OR^{bb1},
(4) —CH₂—CH₂—C(═O)—OR^{bb2},
(5) —CH₂—O—CH₂—C(═O)—OR^{bb3},
(6) hydrogen atom;
R^{bb1}, R^{bb2}, and R^{bb3} are each independently hydrogen atom or C₁₋₆ alkyl group;
R^{c} is selected from the following (1) to (15):
(1) —(CH₂)ₙ₃-ring P,
(2) —(CH₂)ₙ₄—C(═O)—NH—S(═O)₂—CH₃,
(3) C₁₋₆ alkyl group which may optionally be substituted with the same or different 1 to 5 substituents selected from Group C,
(4) C₃₋₆ cycloalkyl group which may optionally be substituted with the same or different 1 to 5 substituents selected from Group C,
(5) C₂₋₁₂ alkenyl group,
(6) C₂₋₁₂ alkynyl group,
(7) —NR^{cc3}R^{cc4},
(8) —OR^{cc5},
(9) —O—CH₂CH₂—OH,
(10) —O—CH₂C(═O)NH—CH₃,
R^{cc3} and R^{cc4} are each independently,
hydrogen atom,
C₁₋₆ alkyl group,
C₁₋₆ alkylcarbonyl group,
benzyloxycarbonyl group, or
C₁₋₆ alkyl group which is substituted with 1 to 3 hydroxyl groups;
R^{cc5} is
hydrogen atom,
C₁₋₆ alkyl group which may optionally be substituted with the same or different 1 to 5 substituents selected from Group C, or
benzyl group;
ring P is monocyclic heterocycle which is a carboxylic acid equivalent wherein the carboxylic acid equivalent may optionally be substituted;
n3 is an integer selected from 1 to 3; and
n4 is an integer selected from 1 to 3;
each R^{d} is the same or different and selected from the following (1) to (13):
(1) halogen atom,
(2) C₁₋₆ alkyl group which may optionally be substituted with the same or different 1 to 5 substituents selected from Group B,
(3) C₃₋₇ cycloalkyl group which may optionally be substituted with the same or different 1 to 5 substituents selected from Group B,
(4) C₂₋₆ alkenyl group,
(5) cyano group,
(6) —C(═O)—OR^{dd1},
(7) —C(═O)—NR^{dd2}R^{dd3},
(8) —OR^{dd4},
(9) —NR^{dd5}—C(═O)—R^{dd6},
(10) —NR^{dd7}—C(═O)—NR^{dd8}R^{dd9},
(11) —NR^{dd10}—S(═O)₂—R^{dd11},
(12) —NR^{dd12}—S(═O)₂—NR^{dd13}R^{dd14},
(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
R^{e} is hydrogen atom or C₁₋₆ alkyl group;
Q is selected from the following (1) to (9):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,
(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;
Y is selected from the following (1) to (3):
(1) single bond,
(2) —S(═O)₂—,
(3) C₁₋₃ alkylene which may optionally be substituted with 1 to 3 hydroxyl groups;
m is each independently an integer selected from 0 or 1 to 5;
Group A consists of the following (a) to (m):
(a) C₁₋₇ alkyl group which may optionally be substituted with the same or different 1 to 5 substituents selected from Group AA,
(b) halogen atom,
(c) phenyl group,
(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,
(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(g) C₃₋₇ cycloalkyl group which may optionally be substituted with the same or different 1 to 5 substituents selected from Group AA,
(h) —C(═O)—NR^{A1}R^{A2},
(i) —C(═O)—OR^{A3},
(j) —C(═O)—R^{A4},
(k) —OR^{A5},
(l) —NR^{A6}R^{A7},
(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
hydrogen atom,
C₁₋₆ alkyl group which may optionally be substituted with the same or different 1 to 6 halogen atoms,
C₃₋₇ cycloalkyl group which may optionally be substituted with the same or different 1 to 5 substituents selected from Group AA,
benzyl group which may optionally be substituted with the same or different 1 to 5 substituents selected from Group AA,
phenyl group which may optionally be substituted with the same or different 1 to 5 substituents selected from Group AA,
C₁₋₆ alkylcarbonyl group,
C₁₋₆ alkylsulfonyl group, or
C₂₋₆ alkenyl group; and
R^{A8} is each independently C₁₋₆ alkyl group;
Group AA consists of the following (a) to (o):
(a) halogen atom,
(b) C₁₋₃ alkyl group which may optionally be substituted with the same or different 1 to 6 halogen atoms,
(c) —OR^{AA1},
(d) —C(═O)—NR^{AA2}R^{AA3},
(e) —C(═O)—OR^{AA4},
(f) —O—C(═O)—R^{AA5},
(g) —C(═O)—R^{AA6},
(h) ═O,
(i) C₃₋₇ cycloalkyl group,
(j) phenyl group which may optionally be substituted with the same or different 1 to 5 C₁₋₃ alkyl groups,
(k) —NR^{AA7}R^{AA8},
(l) —NR^{AA9}—C(═O)—R^{AA10},
(m) —NR^{AA11}—C(═O)—NR^{AA12}R^{AA13},
(n) —NR^{AA14}—S(═O)₂—R^{AA15},
(o) —NR^{AA16}—S(═O)₂—NR^{AA17}R^{AA18};
R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):
(a) halogen atom,
(b) C₃₋₇ cycloalkyl group
(c) —OR^{B1},
(d) —C(═O)—NR^{B2}R^{B3},
(e) —C(═O)—OR^{B4},
(f) C₁₋₆ alkyl group,
(g) —NR^{B5}R^{B6},
(h) —NR^{B7}—C(═O)—R^{B8},
(i) —NR^{B9}—C(═O)—NR^{B10}R^{B11},
(j) —NR^{B12}—S(═O)₂—R^{B13},
(k) —NR^{B14}—S(═O)₂—NR^{B15}R^{B16};
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently hydrogen atom or C₁₋₆ alkyl group;
Group C consists of the following (a) to (k):
(a) phenyl group which may optionally be substituted with the same or different 1 to 5 substituents selected from Group CC,
(b) cyano group,
(c) halogen atom,
(d) —OR^{CD1},
(e) —NR^{CD2}R^{CD3},
(f) —C(═O)—NR^{CD4}R^{CD5},
(i) saturated heteromonocyclic group which may optionally be substituted with the same or different 1 to 5 substituents selected from Group CC wherein the saturated heteromonocyclic ring comprises the same or different 1 to 4 hetero atoms selected from nitrogen atom, oxygen atom, or sulfur atom besides carbon atom, and is 4 to 6-membered, excluding ring P,
(j) unsaturated heteromonocyclic group which may optionally be substituted with the same or different 1 to 5 substituents selected from Group CC wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered, excluding ring P,
R^{CD1}, R^{CD2}, and R^{CD3} are each independently,
hydrogen atom,
C₁₋₆ alkyl group,
C₁₋₆ alkylcarbonyl group,
benzyl group,
—C(═O)—O—CH₂-phenyl,
—C(═O)—N(CH₃)₂,
—C(═O)—C(OH)(CH₃)₂,
—C(═O)—CH₂—O—CH₃, or
—C(═O)—CH₂—CN; and
R^{CD4} and R^{CD5} are each independently hydrogen atom or C₁₋₆ alkyl group;
Group CC consists of the following (a) to (c):
(a) halogen atom,
(b) C₁₋₃ alkyl group which may optionally be substituted with the same or different 1 to 6 halogen atoms,
(c) —OR^{AA1},
R^{AA1} is each independently hydrogen atom or C₁₋₆ alkyl group;
provided that when R^{a} is
(1) phenyl group which may optionally be substituted with the same or different 1 to 5 substituents selected from Group A,
(2) C₉₋₁₀ fused carbocyclic group which may optionally be substituted with the same or different 1 to 5 substituents selected from Group A, or
(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
Q is selected from the following (1) to (6):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;
and further provided that when R^{b} is hydrogen atom,
Q is selected from the following (1) to (5):
(1) phenyl group,
(2) C₃₋₇ cycloalkyl group,
(3) C₉₋₁₀ fused carbocyclic group,
(4) cross-linked C₅₋₁₂ cycloalkyl group,
(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered].

23. A compound represented by the following formula:
or a pharmaceutically acceptable salt thereof, wherein
is unsaturated heteromonocyclic group selected from the following (i) to (ii):
each R^{au} is the same or different and selected from the following (1) or (2):
(1) C₇₋₁₂ alkyl group,
(2) C₁₋₁₂ alkyl group which is substituted with the same or different 1 to 5 substituents selected from Group AU;
R^{b} is selected from the following (1) to (6):
(1) C₁₋₆ alkyl group which may optionally be substituted with the same or different 1 to 5 halogen atoms,
(2) C₃₋₇ cycloalkyl group which may optionally be substituted with the same or different 1 to 5 substituents selected from the group consisting of halogen atom and C₁₋₆ alkyl group,
(3) —CH═CH—C(═O)—OR^{bb1},
(4) —CH₂—CH₂—C(═O)—OR^{bb2},
(5) —CH₂—O—CH₂—C(═O)—OR^{bb3},
(6) hydrogen atom;
R^{bb1}, R^{bb2}, and R^{bb3} are each independently hydrogen atom or C₁₋₆ alkyl group;
R^{c} is selected from the following (1) or (2):
(1) —(CH₂)ₙ₁—C(═O)—OR^{cc1},
(2) —O—(CH₂)—C(═O)—OR^{cc2},
R^{cc1} and R^{cc2} are each independently hydrogen atom or C₁₋₆ alkyl group;
n1 is an integer selected from 0 or 1 to 3; and
n2 is an integer selected from 0 or 1 to 3;
each R^{d} is the same or different and selected from the following (1) to (13):
(1) halogen atom,
(2) C₁₋₆ alkyl group which may optionally be substituted with the same or different 1 to 5 substituents selected from Group B,
(3) C₃₋₇ cycloalkyl group which may optionally be substituted with the same or different 1 to 5 substituents selected from Group B,
(4) C₂₋₆ alkenyl group,
(5) cyano group,
(6) —C(═O)—OR^{dd1},
(7) —C(═O)—NR^{dd2}R^{dd3},
(8) —OR^{dd4},
(9) —NR^{dd5}—C(═O)—R^{dd6},
(10) —NR^{dd7}—C(═O)—NR^{dd8}R^{dd9},
(11) —NR^{dd10}—S(O)₂—R^{dd11},
(12) —NR^{dd12}—S(═O)₂—NR^{dd13}R^{dd14},
(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
R^{e} is hydrogen atom or C₁₋₆ alkyl group;
Q is selected from the following (1) to (9):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,
(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;
cyclic moiety UU is selected from the following (1) or (2):
(1) C₃₋₇ cycloalkyl group,
(2) phenyl group;
Y is selected from the following (1) to (3):
(1) single bond,
(2) —S(═O)₂—,
(3) C₁₋₃ alkylene which may optionally be substituted with 1 to 3 hydroxyl groups;
m is each independently an integer selected from 0 or 1 to 5;
nu is an integer selected from 0 or 1 to 3;
Group B consists of the following (a) to (k):
(a) halogen atom,
(b) C₃₋₇ cycloalkyl group
(c) —OR^{B1},
(d) —C(═O)—NR^{B2}R^{B3},
(e) —C(═O)—OR^{B4},
(f) C₁₋₆ alkyl group,
(g) —NR^{B5}R^{B6},
(h) —NR^{B7}—C(═O)—R^{B8},
(i) —NR^{B9}—C(═O)—NR^{B10}R^{B11},
(j) —NR^{B12}—S(═O)₂—R^{B13},
(k) —NR^{B14}—S(═O)₂—NR^{B15}R^{B16},
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently hydrogen atom or C₁₋₆ alkyl group;
Group AU consists of the following (a) to (j):
(a) —OR^{cc1},
(b) —C(═O)—OR^{AU2},
(c) —C(═O)—NR^{AU3}R^{AU4},
(d) phenyl group,
(e) C₃₋₇ cycloalkyl group,
(f) —NR^{AU5}R^{AU6},
(g) —NR^{AU7}—C(═O)—R^{AU8},
(h) —NR^{AU9}—C(═O)—NR^{AU10}R^{AU12},
(i) —NR^{AU13}—S(═O)₂—R^{AU14},
(j) —NR^{AU15}—S(═O)₂—NR^{AU16}R^{AU17};
R^{AU1}, R^{AU2}, R^{AU3}, R^{AU4}, R^{AU5}, R^{AU6}, R^{AU7}, R^{AU8}, R^{AU9}, R^{AU10}, R^{AU11}, R^{AU12}, R^{AU13}, R^{AU14}, R^{AU15}, R^{AU16} and R^{AU17} are each independently hydrogen atom or C₁₋₆ alkyl group;
provided that when cyclic moiety UU is phenyl group,
Q is selected from the following (1) to (6):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;
and further provided that when R^{b} is hydrogen atom,
Q is selected from the following (1) to (5):
(1) phenyl group,
(2) C₃₋₇ cycloalkyl group,
(3) C₉₋₁₀ fused carbocyclic group,
(4) cross-linked C₅₋₁₂ cycloalkyl group,
(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered].

24. A compound represented by the following formula:
or a pharmaceutically acceptable salt thereof, wherein
is unsaturated heteromonocyclic group selected from the following (i) to (ii):
R^{a} is selected from the following (1) to (12):
(1) C₁₋₁₂ alkyl group which may optionally be substituted with the same or different 1 to 5 substituents selected from Group A,
(2) C₃₋₇ cycloalkyl group which may optionally be substituted with the same or different 1 to 5 substituents selected from Group A,
(3) C₂₋₁₂ alkenyl group which may optionally be substituted with the same or different 1 to 5 substituents selected from Group A,
(4) C₂₋₁₂ alkynyl group which may optionally be substituted with the same or different 1 to 5 substituents selected from Group A,
(5) C₅₋₁₁ spirocyclic cycloalkyl group which may optionally be substituted with the same or different 1 to 5 substituents selected from Group A,
(6) cross-linked C₅₋₁₂ cycloalkyl group which may optionally be substituted with the same or different 1 to 5 substituents selected from Group A,
(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,
(8) phenyl group which may optionally be substituted with the same or different 1 to 5 substituents selected from Group A,
(9) C₉₋₁₀ fused carbocyclic group which may optionally be substituted with the same or different 1 to 5 substituents selected from Group A,
(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,
(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;
R^{c} is selected from the following (1) or (2):
(1) —(CH₂)ₙ₁—C(═O)—OR^{cc1},
(2) —O—(CH₂)—C(═O)—OR^{cc2},
R^{cc1} and R^{cc2} are each independently hydrogen atom or C₁₋₆ alkyl group;
n1 is an integer selected from 0 or 1 to 3; and
n2 is an integer selected from 0 or 1 to 3;
each R^{d} is the same or different and selected from the following (1) to (13):
(1) halogen atom,
(2) C₁₋₆ alkyl group which may optionally be substituted with the same or different 1 to 5 substituents selected from Group B,
(3) C₃₋₇ cycloalkyl group which may optionally be substituted with the same or different 1 to 5 substituents selected from Group B,
(4) C₂₋₆ alkenyl group,
(5) cyano group,
(6) —C(═O)—OR^{dd1},
(7) —C(═O)—NR^{dd2}R^{dd3},
(8) —OR^{dd4},
(9) —NR^{dd5}—C(═O)—R^{dd6},
(10) —NR^{dd7}—C(═O)—NR^{dd8}R^{dd9},
(11) —NR^{dd10}—S(═O)₂—R^{dd11},
(12) —NR^{dd12}—S(═O)₂—NR^{dd13}R^{dd14},
(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
Re is hydrogen atom or C₁₋₆ alkyl group;
Q is selected from the following (1) to (9):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,
(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;
Y is selected from the following (1) to (3):
(1) single bond,
(2) —S(═O)₂—,
(3) C₁₋₃alkylene which may optionally be substituted with 1 to 3 hydroxyl groups;
m is each independently an integer selected from 0 or 1 to 5;
Group A consists of the following (a) to (m):
(a) C₁₋₇ alkyl group which may optionally be substituted with the same or different 1 to 5 substituents selected from Group AA,
(b) halogen atom,
(c) phenyl group,
(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,
(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(g) C₃₋₇ cycloalkyl group which may optionally be substituted with the same or different 1 to 5 substituents selected from Group AA,
(h) —C(═O)—NR^{A1}R^{A2},
(i) —C(═O)—OR^{A3},
(j) —C(═O)—R^{A4},
(k) —OR^{A5},
(l) —NR^{A6}R^{A7},
(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
hydrogen atom,
C₁₋₆ alkyl group which may optionally be substituted with the same or different 1 to 6 halogen atoms,
C₃₋₇ cycloalkyl group which may optionally be substituted with the same or different 1 to 5 substituents selected from Group AA,
benzyl group which may optionally be substituted with the same or different 1 to 5 substituents selected from Group AA,
phenyl group which may optionally be substituted with the same or different 1 to 5 substituents selected from Group AA,
C₁₋₆ alkylcarbonyl group,
C₁₋₆ alkylsulfonyl group, or
C₂₋₆ alkenyl group; and
R^{A8} is each independently C₁₋₆ alkyl group;
Group AA consists of the following (a) to (o):
(a) halogen atom,
(b) C₁₋₃ alkyl group which may optionally be substituted with the same or different 1 to 6 halogen atoms,
(c) —OR^{AA1},
(d) —C(═O)—NR^{AA2}R^{AA3},
(e) —C(═O)—OR^{AA4},
(f) —O—C(═O)—R^{AA5},
(g) —C(═O)—R^{AA6},
(h) ═O,
(i) C₃₋₇ cycloalkyl group,
(j) phenyl group which may optionally be substituted with the same or different 1 to 5 C₁₋₃ alkyl groups,
(k) —NR^{AA7}R^{AA8},
(l) —NR^{AA9}—C(═O)—R^{AA10},
(m) —NR^{AA11}—C(═O)—NR^{AA12}R^{AA13},
(n) —NR^{AA14}—S(═O)₂—R^{AA15},
(o) —NR^{AA16}—S(═O)₂—NR^{AA17}R^{AA18};
R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):
(a) halogen atom,
(b) C₃₋₇ cycloalkyl group
(c) —OR^{B1},
(d) —C(═O)—NR^{B2}R^{B3},
(e) —C(═O)—OR^{B4},
(f) C₁₋₆ alkyl group,
(g) —NR^{B5}R^{B6},
(h) —NR^{B7}—C(═O)—R^{B8},
(i) —NR^{B9}—C(═O)—NR^{B10}R^{B11},
(j) —NR^{B12}—S(═O)₂—R^{B13},
(k) —NR^{B14}—S(═O)₂—NR^{B15}R^{B16},
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently, hydrogen atom or C₁₋₆ alkyl group;
provided that when R^{a} is
(1) phenyl group which may optionally be substituted with the same or different 1 to 5 substituents selected from Group A,
(2) C₉₋₁₀ fused carbocyclic group which may optionally be substituted with the same or different 1 to 5 substituents selected from Group A, or
(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
Q is selected from the following (1) to (6):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered.

25. A compound represented by the following formula:
or a pharmaceutically acceptable salt thereof, wherein
is unsaturated heteromonocyclic group selected from the following (i) to (ii):
R^{a} is selected from the following (1) to (12):
(1) C₁₋₁₂ alkyl group which may optionally be substituted with the same or different 1 to 5 substituents selected from Group A,
(2) C₃₋₇ cycloalkyl group which may optionally be substituted with the same or different 1 to 5 substituents selected from Group A,
(3) C₂₋₁₂ alkenyl group which may optionally be substituted with the same or different 1 to 5 substituents selected from Group A,
(4) C₂₋₁₂ alkynyl group which may optionally be substituted with the same or different 1 to 5 substituents selected from Group A,
(5) C₅₋₁₁ spirocyclic cycloalkyl group which may optionally be substituted with the same or different 1 to 5 substituents selected from Group A,
(6) cross-linked C₅₋₁₂ cycloalkyl group which may optionally be substituted with the same or different 1 to 5 substituents selected from Group A,
(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,
(8) phenyl group which may optionally be substituted with the same or different 1 to 5 substituents selected from Group A,
(9) C₉₋₁₀ fused carbocyclic group which may optionally be substituted with the same or different 1 to 5 substituents selected from Group A,
(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,
(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;
R^{b} is selected from the following (1) to (6):
(1) C₁₋₆ alkyl group which may optionally be substituted with the same or different 1 to 5 halogen atoms,
(2) C₃₋₇ cycloalkyl group which may optionally be substituted with the same or different 1 to 5 substituents selected from the group consisting of halogen atom and C₁₋₆ alkyl group,
(3) —CH═CH—C(═O)—OR^{bb1},
(4) —CH₂—CH₂—C(═O)—OR^{bb2},
(5) —CH₂—O—CH₂—C(═O)—OR^{bb3},
(6) hydrogen atom;
R^{bb1}, R^{bb2}, and R^{bb3} are each independently hydrogen atom or C₁₋₆ alkyl group;
R^{c} is selected from the following (1) or (2):
(1) —(CH₂)ₙ₁—C(═O)—OR^{cc1},
(2) —O—(CH₂)—C(═O)—OR^{cc2},
R^{cc1} and R^{cc2} are each independently hydrogen atom or C₁₋₆ alkyl group;
n1 is an integer selected from 0 or 1 to 3; and
n2 is an integer selected from 0 or 1 to 3;
each R^{d} is the same or different and selected from the following (1) to (13):
(1) halogen atom,
(2) C₁₋₆ alkyl group which may optionally be substituted with the same or different 1 to 5 substituents selected from Group B,
(3) C₃₋₇ cycloalkyl group which may optionally be substituted with the same or different 1 to 5 substituents selected from Group B,
(4) C₂₋₆ alkenyl group,
(5) cyano group,
(6) —C(═O)—OR^{dd1},
(7) —C(═O)—NR^{dd2}R^{dd3},
(8) —OR^{dd4},
(9) —NR^{dd5}—C(═O)—R^{dd6},
(10) —NR^{dd7}—C(═O)—NR^{dd8}R^{dd9},
(11) —NR^{dd10}—S(═O)₂—R^{dd11},
(12) —NR^{dd12}—S(═O)₂—NR^{dd13}R^{dd14},
(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
R^{e} is hydrogen atom or C₁₋₆ alkyl group;
Q is selected from the following (1) to (9):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,
(7) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(8) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(9) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;
m is each independently an integer selected from 0 or 1 to 5;
Group A consists of the following (a) to (m):
(a) C₁₋₇ alkyl group which may optionally be substituted with the same or different 1 to 5 substituents selected from Group AA,
(b) halogen atom,
(c) phenyl group,
(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,
(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(g) C₃₋₇ cycloalkyl group which may optionally be substituted with the same or different 1 to 5 substituents selected from Group AA,
(h) —C(═O)—NR^{A1}R^{A2},
(i) —C(═O)—OR^{A3},
(j) —C(═O)—R^{A4},
(k) —OR^{A5},
(l) —NR^{A6}R^{A7},
(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
hydrogen atom,
C₁₋₆ alkyl group which may optionally be substituted with the same or different 1 to 6 halogen atoms,
C₃₋₇ cycloalkyl group which may optionally be substituted with the same or different 1 to 5 substituents selected from Group AA,
benzyl group which may optionally be substituted with the same or different 1 to 5 substituents selected from Group AA,
phenyl group which may optionally be substituted with the same or different 1 to 5 substituents selected from Group AA,
C₁₋₆ alkylcarbonyl group,
C₁₋₆ alkylsulfonyl group, or
C₂₋₆ alkenyl group; and
R^{A8} is each independently C₁₋₆ alkyl group;
Group AA consists of the following (a) to (o):
(a) halogen atom,
(b) C₁₋₃ alkyl group which may optionally be substituted with the same or different 1 to 6 halogen atoms,
(c) —OR^{AA1},
(d) —C(═O)—NR^{AA2}R^{AA3},
(e) —C(═O)—OR^{AA4},
(f) —O—C(═O)—R^{AA5},
(g) —C(═O)—R^{AA6},
(h) ═O,
(i) C₃₋₇ cycloalkyl group,
(j) phenyl group which may optionally be substituted with the same or different 1 to 5 C₁₋₃ alkyl groups,
(k) —NR^{AA7}R^{AA8},
(l) —NR^{AA9}—C(═O)—R^{AA10},
(m) —NR^{AA11}—C(═O)—NR^{AA12}R^{AA13},
(n) —NR^{AA14}—S(O)₂—R^{AA15},
(o) —NR^{AA16}—S(═O)₂—NR^{A17}R^{AA18};
R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):
(a) halogen atom,
(b) C₃₋₇ cycloalkyl group
(c) —OR^{B1},
(d) —C(═O)—NR^{B2}R^{B3},
(e) —C(═O)—OR^{B4},
(f) C₁₋₆ alkyl group,
(g) —NR^{B5}R^{B6},
(h) —NR^{B7}—C(═O)—R^{B8},
(i) —NR^{B9}—C(═O)—NR^{B10}R^{B11},
(j) —NR^{B12}—S(═O)₂—R^{B13},
(k) —NR^{B14}—S(═O)₂—NR^{B15}R^{B16},
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently, hydrogen atom or C₁₋₆ alkyl group;
provided that when R^{a} is
(1) phenyl group which may optionally be substituted with the same or different 1 to 5 substituents selected from Group A,
(2) C₆₋₁₀ fused carbocyclic group which may optionally be substituted with the same or different 1 to 5 substituents selected from Group A, or
(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
Q is selected from the following (1) to (6):
(1) phenyl group,
(2) C₃₋₇ cycloalkyl group,
(3) C₉₋₁₀ fused carbocyclic group,
(4) cross-linked C₅₋₁₂ cycloalkyl group,
(5) C₃₋₈ alkyl group,
(6) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;
and further provided that when R^{b} is hydrogen atom,
Q is selected from the following (1) to (5):
(1) phenyl group,
(2) C₃₋₇ cycloalkyl group,
(3) C₉₋₁₀ fused carbocyclic group,
(4) cross-linked C₅₋₁₂ cycloalkyl group,
(5) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered.

26. A compound represented by the following formula:
or a pharmaceutically acceptable salt thereof, wherein
is unsaturated heteromonocyclic group selected from the following (i) to (ii):
R^{a} is selected from the following (1) to (12):
(1) C₁₋₁₂ alkyl group which may optionally be substituted with the same or different 1 to 5 substituents selected from Group A,
(2) C₃₋₇ cycloalkyl group which may optionally be substituted with the same or different 1 to 5 substituents selected from Group A,
(3) C₂₋₁₂ alkenyl group which may optionally be substituted with the same or different 1 to 5 substituents selected from Group A,
(4) C₂₋₁₂ alkynyl group which may optionally be substituted with the same or different 1 to 5 substituents selected from Group A,
(5) C₅₋₁₁ spirocyclic cycloalkyl group which may optionally be substituted with the same or different 1 to 5 substituents selected from Group A,
(6) cross-linked C₅₋₁₂ cycloalkyl group which may optionally be substituted with the same or different 1 to 5 substituents selected from Group A,
(7) saturated heteromonocyclic group which may optionally be substituted with the same or different 1 to 5 substituents selected from Group A wherein the saturated heteromonocyclic ring comprises the same or different 1 to 4 hetero atoms selected from nitrogen atom, oxygen atom, or sulfur atom besides carbon atom, and is 4 to 6-membered,
(8) phenyl group which may optionally be substituted with the same or different 1 to 5 substituents selected from Group A,
(9) C₉₋₁₀ fused carbocyclic group which may optionally be substituted with the same or different 1 to 5 substituents selected from Group A,
(10) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(11) saturated fused heterocyclic group which may optionally be substituted with the same or different 1 to 5 substituents selected from Group A wherein the saturated fused heterocyclic ring comprises the same or different 1 to 4 hetero atoms selected from nitrogen atom, oxygen atom, or sulfur atom besides carbon atom, and is 8 to 10-membered,
(12) unsaturated fused heterocyclic group which may optionally be substituted with the same or different 1 to 5 substituents selected from Group A wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered;
R^{b} is selected from the following (1) to (6):
(1) C₁₋₆ alkyl group which may optionally be substituted with the same or different 1 to 5 halogen atoms,
(2) C₃₋₇ cycloalkyl group which may optionally be substituted with the same or different 1 to 5 substituents selected from the group consisting of halogen atom and C₁₋₆ alkyl group,
(3) —CH═CH—C(═O)—OR^{bb1},
(4) —CH₂—CH₂—C(═O)—OR^{bb2},
(5) —CH₂—O—CH₂—C(═O)—OR^{bb3},
(6) hydrogen atom;
R^{bb1}, R^{bb2}, and R^{bb3} are each independently hydrogen atom or C₁₋₆ alkyl group;
R^{c} is selected from the following (1) or (2):
(1) —(CH₂)ₙ₁—C(═O)—OR^{cc1},
(2) —O—(CH₂)—C(═O)—OR^{cc2},
R^{cc1} and R^{cc2} are each independently hydrogen atom or C₁₋₆ alkyl group;
n1 is an integer selected from 0 or 1 to 3; and
n2 is an integer selected from 0 or 1 to 3;
each R^{d} is the same or different and selected from the following (1) to (13):
(1) halogen atom,
(2) C₁₋₆ alkyl group which may optionally be substituted with the same or different 1 to 5 substituents selected from Group B,
(3) C₃₋₇ cycloalkyl group which may optionally be substituted with the same or different 1 to 5 substituents selected from Group B,
(4) C₂₋₆ alkenyl group,
(5) cyano group,
(6) —C(═O)—OR^{dd1},
(7) —C(═O)—NR^{dd2}R^{dd3},
(8) —OR^{dd4},
(9) —NR^{dd5}—C(═O)—R^{dd6},
(10) —NR^{dd7}—C(═O)—NR^{dd8}R^{dd9},
(11) —NR^{dd10}—S(═O)₂—R^{dd11},
(12) —NR^{dd12}—S(═O)₂—NR^{dd13}R^{dd14},
(13) —NR^{dd15}R^{dd16};
R^{dd1}, R^{dd2}, R^{dd3}, R^{dd4}, R^{dd5}, R^{dd6}, R^{dd7}, R^{dd8}, R^{dd9}, R^{dd10}, R^{dd11}, R^{dd12}, R^{dd13}, R^{dd14}, R^{dd15}, and R^{dd16} are each independently hydrogen atom or C₁₋₆ alkyl group which may optionally be substituted with the same or different 1 to 5 substituents selected from Group B;
R^{e} is hydrogen atom or C₁₋₆ alkyl group;
Q is selected from the following (1) to (7):
(1) C₃₋₇ cycloalkyl group,
(2) C₉₋₁₀ fused carbocyclic group selected from the group consisting of indanyl group and 1,2,3,4-tetrahydronaphthyl group,
(3) cross-linked C₅₋₁₂ cycloalkyl group,
(4) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered,
(5) unsaturated fused heterocyclic group wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(6) unsaturated heteromonocyclic group wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(7) saturated fused heterocyclic group wherein the saturated fused heterocyclic ring comprises the same or different 1 to 4 hetero atoms selected from nitrogen atom, oxygen atom, or sulfur atom besides carbon atom, and is 8 to 10-membered;
Y is selected from the following (1) to (3):
(1) single bond,
(2) —S(═O)₂—,
(3) C₁₋₃ alkylene which may optionally be substituted with 1 to 3 hydroxyl groups;
m is each independently an integer selected from 0 or 1 to 5;
Group A consists of the following (a) to (m):
(a) C₁₋₇ alkyl group which may optionally be substituted with the same or different 1 to 5 substituents selected from Group AA,
(b) halogen atom,
(c) phenyl group,
(d) saturated heteromonocyclic group which may optionally be substituted with the same or different 1 to 5 substituents selected from Group AA wherein the saturated heteromonocyclic ring comprises the same or different 1 to 4 hetero atoms selected from nitrogen atom, oxygen atom, or sulfur atom besides carbon atom, and is 4 to 6-membered,
(e) unsaturated fused heterocyclic group which may optionally be substituted with the same or different 1 to 5 substituents selected from Group AA wherein the unsaturated fused heterocyclic ring has an unsaturated bond, comprises the same or different 1 to 4 hetero atoms selected from nitrogen atom, oxygen atom, or sulfur atom besides carbon atom, and is 8 to 10-membered,
(f) unsaturated heteromonocyclic group which may optionally be substituted with the same or different 1 to 5 substituents selected from Group AA wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
(g) C₃₋₇ cycloalkyl group which may optionally be substituted with the same or different 1 to 5 substituents selected from Group AA,
(h) —C(═O)—NR^{A1}R^{A2},
(i) —C(═O)—OR^{A3},
(j) —C(═O)—R^{A4},
(k) —OR^{A5},
(l) —NR^{A6}R^{A7},
(m) —S(═O)₂—R^{A8};
R^{A1}, R^{A2}, R^{A3}, and R^{A4} are each independently hydrogen atom or C₁₋₆ alkyl group;
R^{A5}, R^{A6}, and R^{A7} are each independently:
hydrogen atom,
C₁₋₆ alkyl group which may optionally be substituted with the same or different 1 to 6 halogen atoms,
C₃₋₇ cycloalkyl group which may optionally be substituted with the same or different 1 to 5 substituents selected from Group AA,
benzyl group which may optionally be substituted with the same or different 1 to 5 substituents selected from Group AA,
phenyl group which may optionally be substituted with the same or different 1 to 5 substituents selected from Group AA,
C₁₋₆ alkylcarbonyl group,
C₁₋₆ alkylsulfonyl group, or
C₂₋₆ alkenyl group; and
R^{A8} is each independently C₁₋₆ alkyl group;
Group AA consists of the following (a) to (o):
(a) halogen atom,
(b) C₁₋₃ alkyl group which may optionally be substituted with the same or different 1 to 6 halogen atoms,
(c) —OR^{AA1},
(d) —C(═O)—NR^{AA2}R^{AA3},
(e) —C(═O)—OR^{AA4},
(f) —O—C(═O)—R^{AA5},
(g) —C(═O)—R^{AA6},
(h) ═O,
(i) C₃₋₇ cycloalkyl group,
(j) phenyl group which may optionally be substituted with the same or different 1 to 5 C₁₋₃ alkyl groups,
(k) —NR^{AA7}R^{AA8},
(l) —NR^{AA9}—C(═O)—R^{AA10},
(m) —NR^{AA11}—C(═O)—NR^{AA12}R^{AA13},
(n) —NR^{AA14}—S(═O)₂—R^{AA15},
(o) —NR^{AA16}—S(═O)₂—NR^{AA17}R^{AA18};
R^{AA1}, R^{AA2}, R^{AA3}, R^{AA4}, R^{AA5}, R^{AA6}, R^{AA7}, R^{AA8}, R^{AA9}, R^{AA10}, R^{AA11}, R^{AA12}, R^{AA13}, R^{AA14}, R^{AA15}, R^{AA16}, R^{AA17}, and R^{AA18} are each independently hydrogen atom or C₁₋₆ alkyl group;
Group B consists of the following (a) to (k):
(a) halogen atom,
(b) C₃₋₇ cycloalkyl group
(c) —OR^{B1},
(d) —C(═O)—NR^{B2}R^{B3},
(e) —C(═O)—OR^{B4},
(f) C₁₋₆ alkyl group,
(g) —NR^{B5}R^{B6},
(h) —NR^{B7}—C(═O)—R^{B8},
(i) —NR^{B9}—C(═O)—NR^{B10}R^{B11},
(j) —NR^{B12}—S(═O)₂—R^{B13},
(k) —NR^{B14}—S(═O)₂—NR^{B15}R^{B16},
R^{B1}, R^{B2}, R^{B3}, R^{B4}, R^{B5}, R^{B6}, R^{B7}, R^{B8}, R^{B9}, R^{B10}, R^{B11}, R^{B12}, R^{B13}, R^{B14}, R^{B15}, and R^{B16} are each independently, hydrogen atom or C₁₋₆ alkyl group;
provided that when R^{a} is
(1) phenyl group which may optionally be substituted with the same or different 1 to 5 substituents selected from Group A,
(2) C₆₋₁₀ fused carbocyclic group which may optionally be substituted with the same or different 1 to 5 substituents selected from Group A, or
(3) unsaturated heteromonocyclic group which may optionally be substituted with the same or different 1 to 5 substituents selected from Group A wherein the unsaturated heteromonocyclic ring has an unsaturated bond, comprises the same or different 1 to 4 hetero atoms selected from nitrogen atom, oxygen atom, or sulfur atom besides carbon atom, and is 5 to 6-membered,
Q is selected from the following (1) to (4):
(1) C₃₋₇ cycloalkyl group,
(2) C₉₋₁₀ fused carbocyclic group selected from the group consisting of indanyl group and 1,2,3,4-tetrahydronaphthyl group,
(3) cross-linked C₅₋₁₂ cycloalkyl group,
(4) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered;
and further provided that when R^{b} is hydrogen atom,
Q is selected from the following (1) to (4):
(1) C₃₋₇ cycloalkyl group,
(2) C₉₋₁₀ fused carbocyclic group selected from the group consisting of indanyl group and 1,2,3,4-tetrahydronaphthyl group,
(3) cross-linked C₅₋₁₂ cycloalkyl group,
(4) saturated heteromonocyclic group wherein the saturated heteromonocyclic ring comprises the same or different 1 to 4 hetero atoms selected from nitrogen atom, oxygen atom, or sulfur atom besides carbon atom, and is 4 to 6-membered.
